SEQUENCE LISTING (303 sequences)

SEQ ID NO: 1 - protein, 5 aa, Artificial Sequence
  Description of Artificial Sequence Synthetic peptide
  VHH1 and VHH2 CDR1 (Kabat)
SYRMG

SEQ ID NO: 2 - protein, 5 aa, Artificial Sequence
  Description of Artificial Sequence Synthetic peptide
  VHH3 CDR1 (Kabat)
INVMG

SEQ ID NO: 3 - protein, 5 aa, Artificial Sequence
  Description of Artificial Sequence Synthetic peptide
  VHH4 CDR1 (Kabat)
SNAMG

SEQ ID NO: 4 - protein, 5 aa, Artificial Sequence
  Description of Artificial Sequence Synthetic peptide
  VHH5 CDR1 (Kabat)
SYAMG

SEQ ID NO: 5 - protein, 5 aa, Artificial Sequence
  Description of Artificial Sequence Synthetic peptide
  VHH6 CDR1 (Kabat)
SDAMG

SEQ ID NO: 6 - protein, 5 aa, Artificial Sequence
  Description of Artificial Sequence Synthetic peptide
  VHH7 CDR1 (Kabat)
INVMG

SEQ ID NO: 7 - protein, 5 aa, Artificial Sequence
  Description of Artificial Sequence Synthetic peptide
  VHH9 CDR1 (Kabat)
TYRMG

SEQ ID NO: 8 - protein, 5 aa, Artificial Sequence
  Description of Artificial Sequence Synthetic peptide
  VHH10 CDR1 (Kabat)
RYAMG

SEQ ID NO: 9 - protein, 7 aa, Artificial Sequence
  Description of Artificial Sequence Synthetic peptide
  VHH12 CDR1 (Kabat)
FNTYAMG

SEQ ID NO: 10 - protein, 7 aa, Artificial Sequence
  Description of Artificial Sequence Synthetic peptide
  VHH1 and VHH2 CDR1 (Chothia)
GLTFSSY

SEQ ID NO: 11 - protein, 7 aa, Artificial Sequence
  Description of Artificial Sequence Synthetic peptide
  VHH3 CDR1 (Chothia)
GSIFSIN

SEQ ID NO: 12 - protein, 7 aa, Artificial Sequence
  Description of Artificial Sequence Synthetic peptide
  VHH4 CDR1 (Chothia)
GTSVSSN

SEQ ID NO: 13 - protein, 7 aa, Artificial Sequence
  Description of Artificial Sequence Synthetic peptide
  VHH5 CDR1 (Chothia)
GRTFSSY

SEQ ID NO: 14 - protein, 7 aa, Artificial Sequence
  Description of Artificial Sequence Synthetic peptide
  VHH6 CDR1 (Chothia)
GSSVSSD

SEQ ID NO: 15 - protein, 7 aa, Artificial Sequence
  Description of Artificial Sequence Synthetic peptide
  VHH7 CDR1 (Chothia)
RSIGSIN

SEQ ID NO: 16 - protein, 7 aa, Artificial Sequence
  Description of Artificial Sequence Synthetic peptide
  VHH9 CDR1 (Chothia)
GRTFSTY

SEQ ID NO: 17 - protein, 7 aa, Artificial Sequence
  Description of Artificial Sequence Synthetic peptide
  VHH10 CDR1 (Chothia)
GFTFTRY

SEQ ID NO: 18 - protein, 7 aa, Artificial Sequence
  Description of Artificial Sequence Synthetic peptide
  VHH11 CDR1 (Chothia)
GRTFTTY

SEQ ID NO: 19 - protein, 9 aa, Artificial Sequence
  Description of Artificial Sequence Synthetic peptide
  VHH12 CDR1 (Chothia)
GRTLSFNTY

SEQ ID NO: 20 - protein, 8 aa, Artificial Sequence
  Description of Artificial Sequence Synthetic peptide
  VHH1 and VHH2 CDR1 (IMGT)
GLTFSSYR

SEQ ID NO: 21 - protein, 8 aa, Artificial Sequence
  Description of Artificial Sequence Synthetic peptide
  VHH3 CDR1 (IMGT)
GSIFSINV

SEQ ID NO: 22 - protein, 8 aa, Artificial Sequence
  Description of Artificial Sequence Synthetic peptide
  VHH4 CDR1 (IMGT)
GTSVSSNA

SEQ ID NO: 23 - protein, 8 aa, Artificial Sequence
  Description of Artificial Sequence Synthetic peptide
  VHH5 CDR1 (IMGT)
GRTFSSYA

SEQ ID NO: 24 - protein, 8 aa, Artificial Sequence
  Description of Artificial Sequence Synthetic peptide
  VHH6 CDR1 (IMGT)
GSSVSSDA

SEQ ID NO: 25 - protein, 8 aa, Artificial Sequence
  Description of Artificial Sequence Synthetic peptide
  VHH7 CDR1 (IMGT)
RSIGSINV

SEQ ID NO: 26 - protein, 8 aa, Artificial Sequence
  Description of Artificial Sequence Synthetic peptide
  VHH9 CDR1 (IMGT)
GRTFSTYR

SEQ ID NO: 27 - protein, 8 aa, Artificial Sequence
  Description of Artificial Sequence Synthetic peptide
  VHH10 CDR1 (IMGT)
GFTFTRYA

SEQ ID NO: 28 - protein, 8 aa, Artificial Sequence
  Description of Artificial Sequence Synthetic peptide
  VHH11 CDR1 (IMGT)
GRTFTTYR

SEQ ID NO: 29 - protein, 10 aa, Artificial Sequence
  Description of Artificial Sequence Synthetic peptide
  VHH12 CDR1 (IMGT)
GRTLSFNTYA

SEQ ID NO: 30 - protein, 20 aa, Artificial Sequence
  Description of Artificial Sequence Synthetic peptide
  VHH1 and VHH2 CDR2 (Kabat)
AIDWNGRGTYYRYYADSVKG

SEQ ID NO: 31 - protein, 16 aa, Artificial Sequence
  Description of Artificial Sequence Synthetic peptide
  VHH3 CDR2 (Kabat)
RINGGGITHYAESVKG

SEQ ID NO: 32 - protein, 16 aa, Artificial Sequence
  Description of Artificial Sequence Synthetic peptide
  VHH4 CDR2 (Kabat)
FIDRIATTTIATSVKG

SEQ ID NO: 33 - protein, 17 aa, Artificial Sequence
  Description of Artificial Sequence Synthetic peptide
  VHH5 CDR2 (Kabat)
AITWNGGTTYYADSVKG

SEQ ID NO: 34 - protein, 16 aa, Artificial Sequence
  Description of Artificial Sequence Synthetic peptide
  VHH6 CDR2 (Kabat)
FISGGGTTTYADSVKG

SEQ ID NO: 35 - protein, 16 aa, Artificial Sequence
  Description of Artificial Sequence Synthetic peptide
  VHH7 CDR2 (Kabat)
RITGGGSTHYAESVKG

SEQ ID NO: 36 - protein, 17 aa, Artificial Sequence
  Description of Artificial Sequence Synthetic peptide
  VHH9 CDR2 (Kabat)
AISWSGGSTTYADPVKG

SEQ ID NO: 37 - protein, 17 aa, Artificial Sequence
  Description of Artificial Sequence Synthetic peptide
  VHH10 CDR2 (Kabat)
AISWSGSSAGYGDSVKG

SEQ ID NO: 38 - protein, 17 aa, Artificial Sequence
  Description of Artificial Sequence Synthetic peptide
  VHH11 CDR2 (Kabat)
AIRWSGGRTLYADSVKG

SEQ ID NO: 39 - protein, 17 aa, Artificial Sequence
  Description of Artificial Sequence Synthetic peptide
  VHH12 CDR2 (Kabat)
SITWNGGSTSYADSVKG

SEQ ID NO: 40 - protein, 9 aa, Artificial Sequence
  Description of Artificial Sequence Synthetic peptide
  VHH1 and VHH2 CDR2 (Chothia)
DWNGRGTYY

SEQ ID NO: 41 - protein, 5 aa, Artificial Sequence
  Description of Artificial Sequence Synthetic peptide
  VHH3 CDR2 (Chothia)
NGGGI

SEQ ID NO: 42 - protein, 5 aa, Artificial Sequence
  Description of Artificial Sequence Synthetic peptide
  VHH4 CDR2 (Chothia)
DRIAT

SEQ ID NO: 43 - protein, 6 aa, Artificial Sequence
  Description of Artificial Sequence Synthetic peptide
  VHH5 CDR2 (Chothia)
TWNGGT

SEQ ID NO: 44 - protein, 5 aa, Artificial Sequence
  Description of Artificial Sequence Synthetic peptide
  VHH6 CDR2 (Chothia)
SGGGT

SEQ ID NO: 45 - protein, 5 aa, Artificial Sequence
  Description of Artificial Sequence Synthetic peptide
  VHH7 CDR2 (Chothia)
TGGGS

SEQ ID NO: 46 - protein, 6 aa, Artificial Sequence
  Description of Artificial Sequence Synthetic peptide
  VHH9 CDR2 (Chothia)
SWSGGS

SEQ ID NO: 47 - protein, 6 aa, Artificial Sequence
  Description of Artificial Sequence Synthetic peptide
  VHH10 CDR2 (Chothia)
SWSGSS

SEQ ID NO: 48 - protein, 6 aa, Artificial Sequence
  Description of Artificial Sequence Synthetic peptide
  VHH11 CDR2 (Chothia)
RWSGGR

SEQ ID NO: 49 - protein, 6 aa, Artificial Sequence
  Description of Artificial Sequence Synthetic peptide
  VHH12 CDR2 (Chothia)
TWNGGS

SEQ ID NO: 50 - protein, 10 aa, Artificial Sequence
  Description of Artificial Sequence Synthetic peptide
  VHH1 and VHH2 CDR2 (IMGT)
IDWNGRGTYY

SEQ ID NO: 51 - protein, 7 aa, Artificial Sequence
  Description of Artificial Sequence Synthetic peptide
  VHH3 CDR2 (IMGT)
INGGGIT

SEQ ID NO: 52 - protein, 7 aa, Artificial Sequence
  Description of Artificial Sequence Synthetic peptide
  VHH4 CDR2 (IMGT)
IDRIATT

SEQ ID NO: 53 - protein, 8 aa, Artificial Sequence
  Description of Artificial Sequence Synthetic peptide
  VHH5 CDR2 (IMGT)
ITWNGGTT

SEQ ID NO: 54 - protein, 7 aa, Artificial Sequence
  Description of Artificial Sequence Synthetic peptide
  VHH6 CDR2 (IMGT)
ISGGGTT

SEQ ID NO: 55 - protein, 7 aa, Artificial Sequence
  Description of Artificial Sequence Synthetic peptide
  VHH7 CDR2 (IMGT)
ITGGGST

SEQ ID NO: 56 - protein, 8 aa, Artificial Sequence
  Description of Artificial Sequence Synthetic peptide
  VHH9 CDR2 (IMGT)
ISWSGGST

SEQ ID NO: 57 - protein, 8 aa, Artificial Sequence
  Description of Artificial Sequence Synthetic peptide
  VHH10 CDR2 (IMGT)
ISWSGSSA

SEQ ID NO: 58 - protein, 8 aa, Artificial Sequence
  Description of Artificial Sequence Synthetic peptide
  VHH11 CDR2 (IMGT)
IRWSGGRT

SEQ ID NO: 59 - protein, 8 aa, Artificial Sequence
  Description of Artificial Sequence Synthetic peptide
  VHH12 CDR2 (IMGT)
ITWNGGST

SEQ ID NO: 60 - protein, 13 aa, Artificial Sequence
  Description of Artificial Sequence Synthetic peptide
  VHH1 CDR3 (Kabat)
GSIDLNWYGGMDY

SEQ ID NO: 61 - protein, 15 aa, Artificial Sequence
  Description of Artificial Sequence Synthetic peptide
  VHH2 CDR3 (Kabat)
TTVLTDPRVLNEYAT

SEQ ID NO: 62 - protein, 12 aa, Artificial Sequence
  Description of Artificial Sequence Synthetic peptide
  VHH3 CDR3 (Kabat)
DVFGSSGYVETY

SEQ ID NO: 63 - protein, 5 aa, Artificial Sequence
  Description of Artificial Sequence Synthetic peptide
  VHH4 CDR3 (Kabat)
PLTAR

SEQ ID NO: 64 - protein, 7 aa, Artificial Sequence
  Description of Artificial Sequence Synthetic peptide
  VHH5 CDR3 (Kabat)
DPFNQGY

SEQ ID NO: 65 - protein, 5 aa, Artificial Sequence
  Description of Artificial Sequence Synthetic peptide
  VHH6 CDR3 (Kabat)
PLTSR

SEQ ID NO: 66 - protein, 22 aa, Artificial Sequence
  Description of Artificial Sequence Synthetic peptide
  VHH7 CDR3 (Kabat)
MVNPIITAWGTIGVREIPDYDY

SEQ ID NO: 67 - protein, 5 aa, Artificial Sequence
  Description of Artificial Sequence Synthetic peptide
  VHH9 CDR3 (Kabat)
DQRGY

SEQ ID NO: 68 - protein, 7 aa, Artificial Sequence
  Description of Artificial Sequence Synthetic peptide
  VHH10 CDR3 (Kabat)
DPFNQGY

SEQ ID NO: 69 - protein, 21 aa, Artificial Sequence
  Description of Artificial Sequence Synthetic peptide
  VHH11 CDR3 (Kabat)
DLAEYSGTYSSPADSPAGYDY

SEQ ID NO: 70 - protein, 14 aa, Artificial Sequence
  Description of Artificial Sequence Synthetic peptide
  VHH12 CDR3 (Kabat)
ARYYVSGTYFPANY

SEQ ID NO: 71 - protein, 13 aa, Artificial Sequence
  Description of Artificial Sequence Synthetic peptide
  VHH1 CDR3 (Chothia)
GSIDLNWYGGMDY

SEQ ID NO: 72 - protein, 15 aa, Artificial Sequence
  Description of Artificial Sequence Synthetic peptide
  VHH2 CDR3 (Chothia)
TTVLTDPRVLNEYAT

SEQ ID NO: 73 - protein, 12 aa, Artificial Sequence
  Description of Artificial Sequence Synthetic peptide
  VHH3 CDR3 (Chothia)
DVFGSSGYVETY

SEQ ID NO: 74 - protein, 5 aa, Artificial Sequence
  Description of Artificial Sequence Synthetic peptide
  VHH4 CDR3 (Chothia)
PLTAR

SEQ ID NO: 75 - protein, 7 aa, Artificial Sequence
  Description of Artificial Sequence Synthetic peptide
  VHH5 CDR3 (Chothia)
DPFNQGY

SEQ ID NO: 76 - protein, 5 aa, Artificial Sequence
  Description of Artificial Sequence Synthetic peptide
  VHH6 CDR3 (Chothia)
PLTSR

SEQ ID NO: 77 - protein, 22 aa, Artificial Sequence
  Description of Artificial Sequence Synthetic peptide
  VHH7 CDR3 (Chothia)
MVNPIITAWGTIGVREIPDYDY

SEQ ID NO: 78 - protein, 5 aa, Artificial Sequence
  Description of Artificial Sequence Synthetic peptide
  VHH9 CDR3 (Chothia)
DQRGY

SEQ ID NO: 79 - protein, 7 aa, Artificial Sequence
  Description of Artificial Sequence Synthetic peptide
  VHH10 CDR3 (Chothia)
DPFNQGY

SEQ ID NO: 80 - protein, 21 aa, Artificial Sequence
  Description of Artificial Sequence Synthetic peptide
  VHH11 CDR3 (Chothia)
DLAEYSGTYSSPADSPAGYDY

SEQ ID NO: 81 - protein, 14 aa, Artificial Sequence
  Description of Artificial Sequence Synthetic peptide
  VHH12 CDR3 (Chothia)
ARYYVSGTYFPANY

SEQ ID NO: 82 - protein, 16 aa, Artificial Sequence
  Description of Artificial Sequence Synthetic peptide
  VHH1 CDR3 (IMGT)
CAAGSIDLNWYGGMDY

SEQ ID NO: 83 - protein, 18 aa, Artificial Sequence
  Description of Artificial Sequence Synthetic peptide
  VHH2 CDR3 (IMGT)
CAATTVLTDPRVLNEYAT

SEQ ID NO: 84 - protein, 14 aa, Artificial Sequence
  Description of Artificial Sequence Synthetic peptide
  VHH3 CDR3 (IMGT)
KADVFGSSGYVETY

SEQ ID NO: 85 - protein, 7 aa, Artificial Sequence
  Description of Artificial Sequence Synthetic peptide
  VHH4 CDR3 (IMGT)
NHPLTAR

SEQ ID NO: 86 - protein, 9 aa, Artificial Sequence
  Description of Artificial Sequence Synthetic peptide
  VHH5 CDR3 (IMGT)
AADPFNQGY

SEQ ID NO: 87 - protein, 7 aa, Artificial Sequence
  Description of Artificial Sequence Synthetic peptide
  VHH6 CDR3 (IMGT)
NHPLTSR

SEQ ID NO: 88 - protein, 24 aa, Artificial Sequence
  Description of Artificial Sequence Synthetic peptide
  VHH7 CDR3 (IMGT)
ASMVNPIITAWGTIGVREIPDYDY

SEQ ID NO: 89 - protein, 6 aa, Artificial Sequence
  Description of Artificial Sequence Synthetic peptide
  VHH9 CDR3 (IMGT)
NDQRGY

SEQ ID NO: 90 - protein, 9 aa, Artificial Sequence
  Description of Artificial Sequence Synthetic peptide
  VHH10 CDR3 (IMGT)
AADPFNQGY

SEQ ID NO: 91 - protein, 23 aa, Artificial Sequence
  Description of Artificial Sequence Synthetic peptide
  VHH11 CDR3 (IMGT)
AADLAEYSGTYSSPADSPAGYDY

SEQ ID NO: 92 - protein, 16 aa, Artificial Sequence
  Description of Artificial Sequence Synthetic peptide
  VHH12 CDR3 (IMGT)
AAARYYVSGTYFPANY

SEQ ID NO: 93 - protein, 125 aa, Artificial Sequence
  Description of Artificial Sequence Synthetic polypeptide
  VHH1 - VH amino acid sequence
QVQLVESGGGLVQAGGSLKLACAAPGLTFSSYRMGWFRQAPGQEREFVAAIDWNGRGTYYRYYADSVKGRSTISRDNAKNTMYLQMNSLKPEDTAVYYCAAGSIDLNWYGGMDYWGQGTQVTVSS

SEQ ID NO: 94 - protein, 127 aa, Artificial Sequence
  Description of Artificial Sequence Synthetic polypeptide
  VHH2 - VH amino acid sequence
EVQVVESGGGLVQAGGSLKLACAAPGLTFSSYRMGWFRQAPGQEREFVAAIDWNGRGTYYRYYADSVKGRSTISRDNAKNTVYLQMNSLKPEDTAVYYCAATTVLTDPRVLNEYATWGQGTQVTVSS

SEQ ID NO: 95 - protein, 120 aa, Artificial Sequence
  Description of Artificial Sequence Synthetic polypeptide
  VHH3 - VH amino acid sequence
QLQLVESGGGLVQPGGSLRLSCAASGSIFSINVMGWYRQAPGKQRELVARINGGGITHYAESVKGRFTISRDNAKNTVYLQMNSLKPEDTAAYYCKADVFGSSGYVETYWGQGTQVTVSS

SEQ ID NO: 96 - protein, 113 aa, Artificial Sequence
  Description of Artificial Sequence Synthetic polypeptide
  VHH4 - VH amino acid sequence
EVQVVESGGGLVQAGGSLRLSCAVSGTSVSSNAMGWYRQAPGKQREWVGFIDRIATTTIATSVKGRFAITRDNAKNTVYLQMSGLKPEDTAVYYCNHPLTARWGQGTQVTVSS

SEQ ID NO: 97 - protein, 116 aa, Artificial Sequence
  Description of Artificial Sequence Synthetic polypeptide
  VHH5 - VH amino acid sequence
QVQLVESGGGLVQAGGSLRLSCAASGRTFSSYAMGWFRQAPGKEREFVAAITWNGGTTYYADSVKGRFTISRDNAKNTVYLQMNSLKPEDTAVYYCAADPFNQGYWGQGTQVTVSS

SEQ ID NO: 98 - protein, 113 aa, Artificial Sequence
  Description of Artificial Sequence Synthetic polypeptide
  VHH6 - VH amino acid sequence
EVQLVESGGGLVQAGGSLRLSCAVSGSSVSSDAMGWYRQAPGNQRAWVAFISGGGTTTYADSVKGRFTISRDNTKNTVYLHMNSLKPEDTAVYYCNHPLTSRWGQGTQVTVSS

SEQ ID NO: 99 - protein, 130 aa, Artificial Sequence
  Description of Artificial Sequence Synthetic polypeptide
  VHH7 - VH amino acid sequence
EVQVVESGGGLVQAGGSLRLACVASRSIGSINVMGWYRQAPGKQRDLVARITGGGSTHYAESVKGRFTISRDNAKNTVYLQMNSLEPEDTAVYYCASMVNPIITAWGTIGVREIPDYDYWGQGTQVTVSS

SEQ ID NO: 100 - protein, 113 aa, Artificial Sequence
  Description of Artificial Sequence Synthetic polypeptide
  VHH9 - VH amino acid sequence
QVQLVESGGGLVQAGGSLRLSCAVSGRTFSTYRMGWFRQAPGKERSFVAAISWSGGSTTYADPVKGRFTISRDNAKNTVYLRMNSLKPEDTAVYYCNDQRGYWGQGTLVTVSS

SEQ ID NO: 101 - protein, 116 aa, Artificial Sequence
  Description of Artificial Sequence Synthetic polypeptide
  VHH10 - VH amino acid sequence
EVQVVESGGGLVQAGGSLRLSCAASGFTFTRYAMGWFRQAPGKERSFVAAISWSGSSAGYGDSVKGRFTISRDNAKNTLYLQMNSLKPEDTAVYYCAADPFNQGYWGQGTQVTVSS

SEQ ID NO: 102 - protein, 130 aa, Artificial Sequence
  Description of Artificial Sequence Synthetic polypeptide
  VHH11 - VH amino acid sequence
EVQVVESGGGLVQAGGSLRLSCAASGRTFTTYRMGWFRQAPGKEREFVAAIRWSGGRTLYADSVKGRFTISRDNAKNTAYLQMNNLRPEDTAVYYCAADLAEYSGTYSSPADSPAGYDYWGQGTQVTVSS

SEQ ID NO: 103 - protein, 125 aa, Artificial Sequence
  Description of Artificial Sequence Synthetic polypeptide
  VHH12 - VH amino acid sequence
QVQLVETGGGLVQAGDSLRLSCAASGRTLSFNTYAMGWFRQAPGKEREFVASITWNGGSTSYADSVKGRFTITRDNAKNTATLRMNSLQPDDTAVYYCAAARYYVSGTYFPANYWGQGTQVTVSS

SEQ ID NO: 104 - protein, 371 aa, Artificial Sequence
  Description of Artificial Sequence Synthetic polypeptide
  VHH1-linker- mono-Fc protein
QVQLVESGGGLVQAGGSLKLACAAPGLTFSSYRMGWFRQAPGQEREFVAAIDWNGRGTYYRYYADSVKGRSTISRDNAKNTMYLQMNSLKPEDTAVYYCAAGSIDLNWYGGMDYWGQGTQVTVSSEPKTPKPQPQPQLQPQPNPTTESKSPKSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSREEMTKNQVSLSCLVKGFYPSDIAVEWESNGQPENNYKTTVPVLDSDGSFRLASYLTVDKSRWQQGNVFSCSVMHEALHNHYTQKSLSLSPGK

SEQ ID NO: 105 - protein, 373 aa, Artificial Sequence
  Description of Artificial Sequence Synthetic polypeptide
  VHH2-linker- mono-Fc protein
EVQVVESGGGLVQAGGSLKLACAAPGLTFSSYRMGWFRQAPGQEREFVAAIDWNGRGTYYRYYADSVKGRSTISRDNAKNTVYLQMNSLKPEDTAVYYCAATTVLTDPRVLNEYATWGQGTQVTVSSEPKTPKPQPQPQLQPQPNPTTESKSPKSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSREEMTKNQVSLSCLVKGFYPSDIAVEWESNGQPENNYKTTVPVLDSDGSFRLASYLTVDKSRWQQGNVFSCSVMHEALHNHYTQKSLSLSPGK

SEQ ID NO: 106 - protein, 366 aa, Artificial Sequence
  Description of Artificial Sequence Synthetic polypeptide
  VHH3-linker- mono-Fc protein
QLQLVESGGGLVQPGGSLRLSCAASGSIFSINVMGWYRQAPGKQRELVARINGGGITHYAESVKGRFTISRDNAKNTVYLQMNSLKPEDTAAYYCKADVFGSSGYVETYWGQGTQVTVSSEPKTPKPQPQPQLQPQPNPTTESKSPKSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSREEMTKNQVSLSCLVKGFYPSDIAVEWESNGQPENNYKTTVPVLDSDGSFRLASYLTVDKSRWQQGNVFSCSVMHEALHNHYTQKSLSLSPGK

SEQ ID NO: 107 - protein, 359 aa, Artificial Sequence
  Description of Artificial Sequence Synthetic polypeptide
  VHH4-linker- mono-Fc protein
  MOD_RES (96)..(96): Any amino acid
  MOD_RES (99)..(99): Any amino acid
EVQVVESGGGLVQAGGSLRLSCAVSGTSVSSNAMGWYRQAPGKQREWVGFIDRIATTTIATSVKGRFAITRDNAKNTVYLQMSGLKPEDTAVYYCXHPXTARWGQGTQVTVSSEPKTPKPQPQPQLQPQPNPTTESKSPKSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSREEMTKNQVSLSCLVKGFYPSDIAVEWESNGQPENNYKTTVPVLDSDGSFRLASYLTVDKSRWQQGNVFSCSVMHEALHNHYTQKSLSLSPGK

SEQ ID NO: 108 - protein, 362 aa, Artificial Sequence
  Description of Artificial Sequence Synthetic polypeptide
  VHH5-linker- mono-Fc protein
QVQLVESGGGLVQAGGSLRLSCAASGRTFSSYAMGWFRQAPGKEREFVAAITWNGGTTYYADSVKGRFTISRDNAKNTVYLQMNSLKPEDTAVYYCAADPFNQGYWGQGTQVTVSSEPKTPKPQPQPQLQPQPNPTTESKSPKSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSREEMTKNQVSLSCLVKGFYPSDIAVEWESNGQPENNYKTTVPVLDSDGSFRLASYLTVDKSRWQQGNVFSCSVMHEALHNHYTQKSLSLSPGK

SEQ ID NO: 109 - protein, 359 aa, Artificial Sequence
  Description of Artificial Sequence Synthetic polypeptide
  VHH6-linker- mono-Fc protein
EVQLVESGGGLVQAGGSLRLSCAVSGSSVSSDAMGWYRQAPGNQRAWVAFISGGGTTTYADSVKGRFTISRDNTKNTVYLHMNSLKPEDTAVYYCNHPLTSRWGQGTQVTVSSEPKTPKPQPQPQLQPQPNPTTESKSPKSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSREEMTKNQVSLSCLVKGFYPSDIAVEWESNGQPENNYKTTVPVLDSDGSFRLASYLTVDKSRWQQGNVFSCSVMHEALHNHYTQKSLSLSPGK

SEQ ID NO: 110 - protein, 376 aa, Artificial Sequence
  Description of Artificial Sequence Synthetic polypeptide
  VHH7-linker- mono-Fc protein
EVQVVESGGGLVQAGGSLRLACVASRSIGSINVMGWYRQAPGKQRDLVARITGGGSTHYAESVKGRFTISRDNAKNTVYLQMNSLEPEDTAVYYCASMVNPIITAWGTIGVREIPDYDYWGQGTQVTVSSEPKTPKPQPQPQLQPQPNPTTESKSPKSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSREEMTKNQVSLSCLVKGFYPSDIAVEWESNGQPENNYKTTVPVLDSDGSFRLASYLTVDKSRWQQGNVFSCSVMHEALHNHYTQKSLSLSPGK

SEQ ID NO: 111 - protein, 359 aa, Artificial Sequence
  Description of Artificial Sequence Synthetic polypeptide
  VHH9-linker- mono-Fc protein
QVQLVESGGGLVQAGGSLRLSCAVSGRTFSTYRMGWFRQAPGKERSFVAAISWSGGSTTYADPVKGRFTISRDNAKNTVYLRMNSLKPEDTAVYYCNDQRGYWGQGTLVTVSSEPKTPKPQPQPQLQPQPNPTTESKSPKSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSREEMTKNQVSLSCLVKGFYPSDIAVEWESNGQPENNYKTTVPVLDSDGSFRLASYLTVDKSRWQQGNVFSCSVMHEALHNHYTQKSLSLSPGK

SEQ ID NO: 112 - protein, 362 aa, Artificial Sequence
  Description of Artificial Sequence Synthetic polypeptide
  VHH10-linker- mono-Fc protein
EVQVVESGGGLVQAGGSLRLSCAASGFTFTRYAMGWFRQAPGKERSFVAAISWSGSSAGYGDSVKGRFTISRDNAKNTLYLQMNSLKPEDTAVYYCAADPFNQGYWGQGTQVTVSSEPKTPKPQPQPQLQPQPNPTTESKSPKSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSREEMTKNQVSLSCLVKGFYPSDIAVEWESNGQPENNYKTTVPVLDSDGSFRLASYLTVDKSRWQQGNVFSCSVMHEALHNHYTQKSLSLSPGK

SEQ ID NO: 113 - protein, 376 aa, Artificial Sequence
  Description of Artificial Sequence Synthetic polypeptide
  VHH11-linker- mono-Fc protein
EVQVVESGGGLVQAGGSLRLSCAASGRTFTTYRMGWFRQAPGKEREFVAAIRWSGGRTLYADSVKGRFTISRDNAKNTAYLQMNNLRPEDTAVYYCAADLAEYSGTYSSPADSPAGYDYWGQGTQVTVSSEPKTPKPQPQPQLQPQPNPTTESKSPKSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSREEMTKNQVSLSCLVKGFYPSDIAVEWESNGQPENNYKTTVPVLDSDGSFRLASYLTVDKSRWQQGNVFSCSVMHEALHNHYTQKSLSLSPGK

SEQ ID NO: 114 - protein, 371 aa, Artificial Sequence
  Description of Artificial Sequence Synthetic polypeptide
  VHH12-linker- mono-Fc protein
QVQLVETGGGLVQAGDSLRLSCAASGRTLSFNTYAMGWFRQAPGKEREFVASITWNGGSTSYADSVKGRFTITRDNAKNTATLRMNSLQPDDTAVYYCAAARYYVSGTYFPANYWGQGTQVTVSSEPKTPKPQPQPQLQPQPNPTTESKSPKSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSREEMTKNQVSLSCLVKGFYPSDIAVEWESNGQPENNYKTTVPVLDSDGSFRLASYLTVDKSRWQQGNVFSCSVMHEALHNHYTQKSLSLSPGK

SEQ ID NO: 115 - protein, 461 aa, Artificial Sequence
  Description of Artificial Sequence Synthetic polypeptide
  IgA Heavy Chain
QVQLVQSGAEVKKPGSSVKVSCKSSGGTSNNYAISWVRQAPGQGLDWMGGISPIFGSTAYAQKFQGRVTISADIFSNTAYMELNSLTSEDTAVYFCARHGNYYYYSGMDVWGQGTTVTVSSASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCPVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 116 - protein, 216 aa, Artificial Sequence
  Description of Artificial Sequence Synthetic polypeptide
  IgA Light Chain
QSALTQPPAVSGTPGQRVTISCSGSDSNIGRRSVNWYQQFPGTAPKLLIYSNDQRPSVVPDRFSGSKSGTSASLAISGLQSEDEAEYYCAAWDDSLKGAVFGGGTQLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 117 - protein, 153 aa, Artificial Sequence
  Description of Artificial Sequence Synthetic polypeptide
  IgA J Chain
SRDSSASASRVAGITAQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 118 - protein, 626 aa, Homo sapiens
  human pIgR extracellular domain (ECD)
KSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGSSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSSRHHHHHH

SEQ ID NO: 119 - protein, 117 aa, Homo sapiens
  human pIgR extracellular domain 1 (D1)
KSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVGSHHHHHH

SEQ ID NO: 120 - protein, 123 aa, Homo sapiens
  human pIgR extracellular domain 2 (D2)
SQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPGSHHHHHH

SEQ ID NO: 121 - protein, 121 aa, Homo sapiens
  human pIgR extracellular domain 3 (D3)
KPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTGSHHHHHH

SEQ ID NO: 122 - protein, 114 aa, Homo sapiens
  human pIgR extracellular domain 5 (D5)
GEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERGSHHHHHH

SEQ ID NO: 123 - protein, 230 aa, Homo sapiens
  human pIgR extracellular domain 1-domain 2 (D1-D2)
KSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPGSHHHHHH

SEQ ID NO: 124 - protein, 228 aa, Homo sapiens
  human pIgR extracellular domain 2-domain 3 (D2-D3)
SQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNGSHHHHHH

SEQ ID NO: 125 - protein, 224 aa, Homo sapiens
  human pIgR extracellular domain 4-domain 5 (D4-D5)
STIPRSPTVVKGVAGSSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERGSHHHHHH

SEQ ID NO: 126 - protein, 7 aa, Oncorhynchus mykiss
  teleost fish pIgR CDR1 of D1
GPQYASY

SEQ ID NO: 127 - protein, 3 aa, Oncorhynchus mykiss
  teleost fish pIgR CDR2 of D1
DAP

SEQ ID NO: 128 - protein, 8 aa, Oncorhynchus mykiss
  teleost fish pIgR CDR3 of D1
VGGVWSAD

SEQ ID NO: 129 - protein, 633 aa, Mus sp.
  mouse pIgR extracellular domain (ECD)
KSPIFGPQEVSSIEGDSVSITCYYPDTSVNRHTRKYWCRQGASGMCTTLISSNGYLSKEYSGRANLINFPENNTFVINIEQLTQDDTGSYKCGLGTSNRGLSFDVSLEVSQVPELPSDTHVYTKDIGRNVTIECPFKRENAPSKKSLCKKTNQSCELVIDSTEKVNPSYIGRAKLFMKGTDLTVFYVNISHLTHNDAGLYICQAGEGPSADKKNVDLQVLAPEPELLYKDLRSSVTFECDLGREVANEAKYLCRMNKETCDVIINTLGKRDPDFEGRILITPKDDNGRFSVLITGLRKEDAGHYQCGAHSSGLPQEGWPIQTWQLFVNEESTIPNRRSVVKGVTGGSVAIACPYNPKESSSLKYWCRWEGDGNGHCPVLVGTQAQVQEEYEGRLALFDQPGNGTYTVILNQLTTEDAGFYWCLTNGDSRWRTTIELQVAEATREPNLEVTPQNATAVLGETFTVSCHYPCKFYSQEKYWCKWSNKGCHILPSHDEGARQSSVSCDQSSQLVSMTLNPVSKEDEGWYWCGVKQGQTYGETTAIYIAVEERTRGSSHVNPTDANARAKVALEEEVVDSSISEKENKAIPNPGPFANEREIQNVGDQAQENRASGDAGSADGQSRSSSSKHHHHHH

SEQ ID NO: 130 - protein, 27 aa, Artificial Sequence
  Description of Artificial Sequence Synthetic peptide
  hinge region (AA)
EPKTPKPQPQPQLQPQPNPTTESKSPK

SEQ ID NO: 131 - DNA, 82 bp, Artificial Sequence
  Description of Artificial Sequence Synthetic oligonucleotide
  hinge region (DNA)
gaacccaagacaccaaaaccacaaccacaaccacaactacaaccacaacccaatcctacaacagaatccaagagccccaaaa

SEQ ID NO: 132 - DNA, 657 bp, Homo sapiens
  Human IgG1 Mono-Fc DNA sequence
agcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccaagcccccatcccgggaggagatgaccaagaaccaggtcagcctgagctgcctggtcaaaggcttctatcccagcgacatcgccgtggagtgggagagcaatgggcagccggagaacaactacaagaccacggtgcccgtgctggactccgacggctccttcagactcgcaagctatctcaccgtggacaagagcagatggcagcaggggaacgtcttctcatgctccgtgatgcatgaggctctgcacaaccactacacgcagaagagcctctccctgtctccgggtaaa

SEQ ID NO: 133 - DNA, 375 bp, Artificial Sequence
  Description of Artificial Sequence Synthetic polynucleotide
  DNA sequence coding for VHH1
  modified_base (349)..(349): a, c, t, g, unknown or other
  modified_base (351)..(351): a, c, t, g, unknown or other
caggtgcagctggtggagtctgggggaggattggtgcaggctgggggctctctgaaactcgcctgtgcagcacctggacttaccttcagttcgtatcgcatgggctggttccgccaggctccagggcaggagcgtgagtttgtagcagctattgattggaatggtcgtggcacatattatcgatactatgcagactccgtgaagggccgatccaccatttccagagacaacgccaagaacacgatgtatctgcaaatgaacagcctgaaacctgaggacacggccgtttattactgtgcagcaggttcgatcgaccttaactggtacggcggcatggactactggggcnangggacccaggtcaccgtctcctca

SEQ ID NO: 134 - DNA, 381 bp, Artificial Sequence
  Description of Artificial Sequence Synthetic polynucleotide
  DNA sequence coding for VHH2
gaggtgcaggtggtggagtctgggggaggattggtgcaggctgggggctctctgaaactcgcctgtgcagcacctggacttaccttcagttcgtatcgcatgggctggttccgccaggctccagggcaggagcgtgagtttgtagcagctattgattggaatggtcgtggcacatattatcgatactatgcagactccgtgaagggccgatccaccatttccagagacaacgccaagaacacggtgtatctgcaaatgaacagcctgaaacctgaggacacggccgtttattactgtgcagctactacggtattaactgaccctagggttcttaatgagtatgccacatggggccaggggacccaggtcaccgtctcctca

SEQ ID NO: 135 - DNA, 360 bp, Artificial Sequence
  Description of Artificial Sequence Synthetic polynucleotide
  DNA sequence coding for VHH3
cagttgcagctcgtggagtctgggggaggcttggtgcagcctggggggtctctgagactctcctgtgcagcctctggaagcatcttcagtatcaatgttatgggctggtaccgccaggctccagggaagcagcgcgagttggtcgcacgtattaatggaggtggcattacacactatgcagagtccgtgaagggccgattcaccatctccagagacaacgccaagaacacggtgtatctgcaaatgaacagcctgaaacctgaggacacagccgcatattactgtaaggcagatgtgttcggtagtagcgggtacgtagaaacctactggggccaggggacccaggtcaccgtctcctca

SEQ ID NO: 136 - DNA, 339 bp, Artificial Sequence
  Description of Artificial Sequence Synthetic polynucleotide
  DNA sequence coding for VHH4
gaggtgcaggtggtggagtctgggggaggcttggtgcaggctgggggctctctgagactctcctgtgcagtctctggaacctccgtcagtagcaatgccatgggttggtaccgccaggctccagggaagcagcgcgagtgggtcggatttattgatcgtattgctaccacgacgattgcaacctccgtgaagggccgattcgccatcaccagagacaacgccaagaacacggtgtatctccaaatgagcggcctgaaacctgaggacacagccgtctattactgtaatcatccattgaccgctcggtggggccaggggacccaggtcaccgtctcctca

SEQ ID NO: 137 - DNA, 348 bp, Artificial Sequence
  Description of Artificial Sequence Synthetic polynucleotide
  DNA sequence coding for VHH5
caggtgcagctggtggagtctgggggaggcttggtgcaggctgggggctctctgagactctcctgtgcagcctctggacgcaccttcagtagctatgccatgggctggttccgccaggctccagggaaggagcgtgagtttgtagcagctattacctggaatggtggtaccacatactatgcagactccgtgaagggccgattcaccatctccagagacaacgccaagaacacggtgtatctgcaaatgaacagcctgaaacctgaggacacggccgtttattactgtgcagcagacccattcaaccaaggctactggggccaggggacccaggtcaccgtctcctca

SEQ ID NO: 138 - DNA, 339 bp, Artificial Sequence
  Description of Artificial Sequence Synthetic polynucleotide
  DNA sequence coding for VHH6
gaggtgcagctcgtggagtctggaggaggcttggtgcaggctggggggtctctgagactctcctgtgcagtctctggaagctccgtcagtagcgatgccatgggttggtaccgccaggctccagggaatcagcgcgcgtgggtcgcatttatttctggtggtggtaccacaacctatgcagactccgttaagggccgattcaccatctccagagacaacaccaagaacacggtgtatctccacatgaacagcctgaaacctgaagacacagccgtctattactgtaatcatccattgacgtctcggtggggccaggggacccaggtcaccgtctcctca

SEQ ID NO: 139 - DNA, 390 bp, Artificial Sequence
  Description of Artificial Sequence Synthetic polynucleotide
  DNA sequence coding for VHH7
gaggtgcaggtggtggagtctgggggaggattggtgcaggctggggggtctctgagactcgcctgtgtagcctctagaagcatcggcagtatcaatgttatgggctggtaccgccaggctccagggaagcagcgcgacttggtcgcacgtattactggaggtggcagtacacactacgcagagtccgtgaagggccgattcaccatctccagagacaacgccaagaacacggtgtatctgcaaatgaacagcctggaacctgaggacacggccgtttattactgtgcgtcaatggtaaaccctatcattacggcttggggtacgattggtgtgcgcgagattcccgactatgactactggggccaggggacccaggtcaccgtctcctca

SEQ ID NO: 140 - DNA, 348 bp, Artificial Sequence
  Description of Artificial Sequence Synthetic polynucleotide
  DNA sequence coding for VHH10
gaggtgcaggtggtggagtctgggggaggcttggtgcaggctggggggtctctgagactctcctgtgcagcctctggattcaccttcacccgctatgccatgggctggttccgccaggctccagggaaggagcgatcgtttgtagcagctattagctggagtggtagtagcgcaggctatggagactccgtgaagggccgattcaccatctccagagacaacgccaagaacacgctgtatctgcaaatgaacagtctaaaacctgaggacacggccgtttattactgtgcagcagacccattcaaccaaggctactggggccaggggacccaggtcaccgtctcctca

SEQ ID NO: 141 - DNA, 390 bp, Artificial Sequence
  Description of Artificial Sequence Synthetic polynucleotide
  DNA sequence coding for VHH11
gaggtgcaggtggtggagtctgggggaggattggtgcaggctgggggctctctgagactctcctgtgcagcctctggacgcaccttcactacctatcgcatgggctggttccgccaggctccagggaaggagcgagagtttgtagcagctattcgctggagtggtggtcgcacattgtatgcagactccgtgaagggccgattcaccatctccagagacaacgccaagaacacagcgtatctgcaaatgaacaacctgagacctgaggacacggccgtttattactgtgcagcagatctagccgagtatagtggtacttactccagccctgcggactcccccgctgggtatgactactggggccaggggacccaggtcaccgtctcctca

SEQ ID NO: 142 - DNA, 375 bp, Artificial Sequence
  Description of Artificial Sequence Synthetic polynucleotide
  DNA sequence coding for VHH12
caggtgcagctggtcgaaactgggggaggattggtgcaggctggggactctctgagactctcctgtgcagcctctggacgcaccctcagcttcaacacctatgccatgggctggttccgccaggctccagggaaggagcgtgaatttgtagcctctattacctggaatggtggaagcacaagctacgcagactccgtgaagggccgattcaccatcaccagagacaacgccaagaacacggctactctgcgaatgaatagcctgcagcccgacgacacggccgtgtattactgtgcagcagcccgatactatgtgagtggtacttacttccccgcgaattactggggccaggggacccaggtcaccgtctcctca

SEQ ID NO: 143 - protein, 31 aa, Homo sapiens
  Exemplary stalk sequence of human pIgR
EKAVADTRDQADGSRASVDSGSSEEQGGSSR

SEQ ID NO: 144 - protein, 33 aa, Mus sp.
  Exemplary stalk sequence of mouse pIgR
EREIQNVGDQAQENRASGDAGSADGQSRSSSSK

SEQ ID NO: 145 - protein, 33 aa, Mus sp.
  Exemplary stalk sequence of mouse pIgR
EREIQNVRDQAQENRASGDAGSADGQSRSSSSK

SEQ ID NO: 146 - protein, 219 aa, Homo sapiens
  Human IgG1 Mono-Fc AA sequence
SPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSREEMTKNQVSLSCLVKGFYPSDIAVEWESNGQPENNYKTTVPVLDSDGSFRLASYLTVDKSRWQQGNVFSCSVMHEALHNHYTQKSLSLSPGK

SEQ ID NO: 147 - protein, 100 aa, Artificial Sequence
  Description of Artificial Sequence Synthetic polypeptide
  Exemplary flexible linker 1
  MISC_FEATURE (1)..(100): This sequence may encompass 1-20 “Glu Ala Ala Ala Lys” repeating units
EAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAK

SEQ ID NO: 148 - protein, 100 aa, Artificial Sequence
  Description of Artificial Sequence Synthetic polypeptide
  Exemplary flexible linker 2
  MISC_FEATURE (1)..(100): This sequence may encompass 1-20 “Gly Gly Gly Gly Ser” repeating units
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 149 - protein, 80 aa, Artificial Sequence
  Description of Artificial Sequence Synthetic polypeptide
  Exemplary flexible linker 3
  MISC_FEATURE (1)..(80): This sequence may encompass 1-20 “Gly Gly Gly Ser” repeating units
GGGSGGGSGGGSGGGSGGGSGGGSGGGSGGGSGGGSGGGSGGGSGGGSGGGSGGGSGGGSGGGSGGGSGGGSGGGSGGGS

SEQ ID NO: 150 - protein, 15 aa, Artificial Sequence
  Description of Artificial Sequence Synthetic peptide
  Exemplary hinge region 1
EPKSCDKTHTCPPCP

SEQ ID NO: 151 - protein, 12 aa, Artificial Sequence
  Description of Artificial Sequence Synthetic peptide
  Exemplary hinge region 2
ERKCCVECPPCP

SEQ ID NO: 152 - protein, 62 aa, Artificial Sequence
  Description of Artificial Sequence Synthetic polypeptide
  Exemplary hinge region 3
ELKTPLGDTTHTCPRCPEPKSCDTPPPCPRCPEPKSCDTPPPCPRCPEPKSCDTPPPCPRCP

SEQ ID NO: 153 - protein, 12 aa, Artificial Sequence
  Description of Artificial Sequence Synthetic peptide
  Exemplary hinge region 4
ESKYGPPCPSCP

SEQ ID NO: 154 - protein, 10 aa, Artificial Sequence
  Description of Artificial Sequence Synthetic peptide
  VHH1 and VHH 2 CDR1 (Exemplary)
GLTFSSYRMG

SEQ ID NO: 155 - protein, 10 aa, Artificial Sequence
  Description of Artificial Sequence Synthetic peptide
  VHH3 CDR1 (Exemplary)
GSIFSINVMG

SEQ ID NO: 156 - protein, 10 aa, Artificial Sequence
  Description of Artificial Sequence Synthetic peptide
  VHH4 CDR1 (Exemplary)
GTSVSSNAMG

SEQ ID NO: 157 - protein, 10 aa, Artificial Sequence
  Description of Artificial Sequence Synthetic peptide
  VHH5 CDR1 (Exemplary)
GRTFSSYAMG

SEQ ID NO: 158 - protein, 10 aa, Artificial Sequence
  Description of Artificial Sequence Synthetic peptide
  VHH6 CDR1 (Exemplary)
GSSVSSDAMG

SEQ ID NO: 159 - protein, 10 aa, Artificial Sequence
  Description of Artificial Sequence Synthetic peptide
  VHH7 CDR1 (Exemplary)
RSIGSINVMG

SEQ ID NO: 160 - protein, 10 aa, Artificial Sequence
  Description of Artificial Sequence Synthetic peptide
  VHH9 CDR1 (Exemplary)
GRTFSTYRMG

SEQ ID NO: 161 - protein, 10 aa, Artificial Sequence
  Description of Artificial Sequence Synthetic peptide
  VHH10 CDR1 (Exemplary)
GFTFTRYAMG

SEQ ID NO: 162 - protein, 10 aa, Artificial Sequence
  Description of Artificial Sequence Synthetic peptide
  VHH11 CDR1 (Exemplary)
GRTFTTYRMG

SEQ ID NO: 163 - protein, 12 aa, Artificial Sequence
  Description of Artificial Sequence Synthetic peptide
  VHH12 CDR1 (Exemplary)
GRTLSFNTYAMG

SEQ ID NO: 164 - protein, 6 aa, Artificial Sequence
  Description of Artificial Sequence Synthetic peptide
  VHH1 and VHH 2 CDR1 (Contact)
SSYRMG

SEQ ID NO: 165 - protein, 6 aa, Artificial Sequence
  Description of Artificial Sequence Synthetic peptide
  VHH3 CDR1 (Contact)
SINVMG

SEQ ID NO: 166 - protein, 6 aa, Artificial Sequence
  Description of Artificial Sequence Synthetic peptide
  VHH4 CDR1 (Contact)
SSNAMG

SEQ ID NO: 167 - protein, 6 aa, Artificial Sequence
  Description of Artificial Sequence Synthetic peptide
  VHH5 CDR1 (Contact)
SSYAMG

SEQ ID NO: 168 - protein, 6 aa, Artificial Sequence
  Description of Artificial Sequence Synthetic peptide
  VHH6 CDR1 (Contact)
SSDAMG

SEQ ID NO: 169 - protein, 6 aa, Artificial Sequence
  Description of Artificial Sequence Synthetic peptide
  VHH7 CDR1 (Contact)
SINVMG

SEQ ID NO: 170 - protein, 6 aa, Artificial Sequence
  Description of Artificial Sequence Synthetic peptide
  VHH9 CDR1 (Contact)
STYRMG

SEQ ID NO: 171 - protein, 6 aa, Artificial Sequence
  Description of Artificial Sequence Synthetic peptide
  VHH10 CDR1 (Contact)
TRYAMG

SEQ ID NO: 172 - protein, 6 aa, Artificial Sequence
  Description of Artificial Sequence Synthetic peptide
  VHH11 CDR1 (Contact)
TTYRMG

SEQ ID NO: 173 - protein, 8 aa, Artificial Sequence
  Description of Artificial Sequence Synthetic peptide
  VHH12 CDR1 (Contact)
SFNTYAMG

SEQ ID NO: 174 - protein, 10 aa, Artificial Sequence
  Description of Artificial Sequence Synthetic peptide
  VHH1 and VHH 2 CDR1 (AbM)
GLTFSSYRMG

SEQ ID NO: 175 - protein, 10 aa, Artificial Sequence
  Description of Artificial Sequence Synthetic peptide
  VHH3 CDR1 (AbM)
GSIFSINVMG

SEQ ID NO: 176 - protein, 10 aa, Artificial Sequence
  Description of Artificial Sequence Synthetic peptide
  VHH4 CDR1 (AbM)
GTSVSSNAMG

SEQ ID NO: 177 - protein, 10 aa, Artificial Sequence
  Description of Artificial Sequence Synthetic peptide
  VHH5 CDR1 (AbM)
GRTFSSYAMG

SEQ ID NO: 178 - protein, 10 aa, Artificial Sequence
  Description of Artificial Sequence Synthetic peptide
  VHH6 CDR1 (AbM)
GSSVSSDAMG

SEQ ID NO: 179 - protein, 10 aa, Artificial Sequence
  Description of Artificial Sequence Synthetic peptide
  VHH7 CDR1 (AbM)
RSIGSINVMG

SEQ ID NO: 180 - protein, 10 aa, Artificial Sequence
  Description of Artificial Sequence Synthetic peptide
  VHH9 CDR1 (AbM)
GRTFSTYRMG

SEQ ID NO: 181 - protein, 10 aa, Artificial Sequence
  Description of Artificial Sequence Synthetic peptide
  VHH10 CDR1 (AbM)
GFTFTRYAMG

SEQ ID NO: 182 - protein, 10 aa, Artificial Sequence
  Description of Artificial Sequence Synthetic peptide
  VHH11 CDR1 (AbM)
GRTFTTYRMG

SEQ ID NO: 183 - protein, 12 aa, Artificial Sequence
  Description of Artificial Sequence Synthetic peptide
  VHH12 CDR1 (AbM)
GRTLSFNTYAMG

SEQ ID NO: 184 - protein, 20 aa, Artificial Sequence
  Description of Artificial Sequence Synthetic peptide
  VHH1 and VHH 2 CDR2 (Exemplary)
AIDWNGRGTYYRYYADSVKG

SEQ ID NO: 185 - protein, 16 aa, Artificial Sequence
  Description of Artificial Sequence Synthetic peptide
  VHH3 CDR2 (Exemplary)
RINGGGITHYAESVKG

SEQ ID NO: 186 - protein, 16 aa, Artificial Sequence
  Description of Artificial Sequence Synthetic peptide
  VHH4 CDR2 (Exemplary)
FIDRIATTTIATSVKG

SEQ ID NO: 187 - protein, 17 aa, Artificial Sequence
  Description of Artificial Sequence Synthetic peptide
  VHH5 CDR2 (Exemplary)
AITWNGGTTYYADSVKG

SEQ ID NO: 188 - protein, 16 aa, Artificial Sequence
  Description of Artificial Sequence Synthetic peptide
  VHH6 CDR2 (Exemplary)
FISGGGTTTYADSVKG

SEQ ID NO: 189 - protein, 16 aa, Artificial Sequence
  Description of Artificial Sequence Synthetic peptide
  VHH7 CDR2 (Exemplary)
RITGGGSTHYAESVKG

SEQ ID NO: 190 - protein, 17 aa, Artificial Sequence
  Description of Artificial Sequence Synthetic peptide
  VHH9 CDR2 (Exemplary)
AISWSGGSTTYADPVKG

SEQ ID NO: 191 - protein, 17 aa, Artificial Sequence
  Description of Artificial Sequence Synthetic peptide
  VHH10 CDR2 (Exemplary)
AISWSGSSAGYGDSVKG

SEQ ID NO: 192 - protein, 17 aa, Artificial Sequence
  Description of Artificial Sequence Synthetic peptide
  VHH11 CDR2 (Exemplary)
AIRWSGGRTLYADSVKG

SEQ ID NO: 193 - protein, 17 aa, Artificial Sequence
  Description of Artificial Sequence Synthetic peptide
  VHH12 CDR2 (Exemplary)
SITWNGGSTSYADSVKG

SEQ ID NO: 194 - protein, 16 aa, Artificial Sequence
  Description of Artificial Sequence Synthetic peptide
  VHH1 and VHH2 CDR2 (Contact)
FVAAIDWNGRGTYYRY

SEQ ID NO: 195 - protein, 12 aa, Artificial Sequence
  Description of Artificial Sequence Synthetic peptide
  VHH3 CDR2 (Contact)
LVARINGGGITH

SEQ ID NO: 196 - protein, 12 aa, Artificial Sequence
  Description of Artificial Sequence Synthetic peptide
  VHH4 CDR2 (Contact)
WVGFIDRIATTT

SEQ ID NO: 197 - protein, 13 aa, Artificial Sequence
  Description of Artificial Sequence Synthetic peptide
  VHH5 CDR2 (Contact)
FVAAITWNGGTTY

SEQ ID NO: 198 - protein, 12 aa, Artificial Sequence
  Description of Artificial Sequence Synthetic peptide
  VHH6 CDR2 (Contact)
WVAFISGGGTTT

SEQ ID NO: 199 - protein, 12 aa, Artificial Sequence
  Description of Artificial Sequence Synthetic peptide
  VHH7 CDR2 (Contact)
LVARITGGGSTH

SEQ ID NO: 200 - protein, 13 aa, Artificial Sequence
  Description of Artificial Sequence Synthetic peptide
  VHH9 CDR2 (Contact)
FVAAISWSGGSTT

SEQ ID NO: 201 - protein, 13 aa, Artificial Sequence
  Description of Artificial Sequence Synthetic peptide
  VHH10 CDR2 (Contact)
FVAAISWSGSSAG

SEQ ID NO: 202 - protein, 13 aa, Artificial Sequence
  Description of Artificial Sequence Synthetic peptide
  VHH11 CDR2 (Contact)
FVAAIRWSGGRTL

SEQ ID NO: 203 - protein, 13 aa, Artificial Sequence
  Description of Artificial Sequence Synthetic peptide
  VHH12 CDR2 (Contact)
FVASITWNGGSTS

SEQ ID NO: 204 - protein, 13 aa, Artificial Sequence
  Description of Artificial Sequence Synthetic peptide
  VHH1 and VHH 2CDR2 (AbM)
AIDWNGRGTYYRY

SEQ ID NO: 205 - protein, 9 aa, Artificial Sequence
  Description of Artificial Sequence Synthetic peptide
  VHH3 CDR2 (AbM)
RINGGGITH

SEQ ID NO: 206 - protein, 9 aa, Artificial Sequence
  Description of Artificial Sequence Synthetic peptide
  VHH4 CDR2 (AbM)
FIDRIATTT

SEQ ID NO: 207 - protein, 10 aa, Artificial Sequence
  Description of Artificial Sequence Synthetic peptide
  VHH5 CDR2 (AbM)
AITWNGGTTY

SEQ ID NO: 208 - protein, 9 aa, Artificial Sequence
  Description of Artificial Sequence Synthetic peptide
  VHH6 CDR2 (AbM)
FISGGGTTT

SEQ ID NO: 209 - protein, 9 aa, Artificial Sequence
  Description of Artificial Sequence Synthetic peptide
  VHH7 CDR2 (AbM)
RITGGGSTH

SEQ ID NO: 210 - protein, 10 aa, Artificial Sequence
  Description of Artificial Sequence Synthetic peptide
  VHH9 CDR2 (AbM)
AISWSGGSTT

SEQ ID NO: 211 - protein, 10 aa, Artificial Sequence
  Description of Artificial Sequence Synthetic peptide
  VHH10 CDR2 (AbM)
AISWSGSSAG

SEQ ID NO: 212 - protein, 10 aa, Artificial Sequence
  Description of Artificial Sequence Synthetic peptide
  VHH11 CDR2 (AbM)
AIRWSGGRTL

SEQ ID NO: 213 - protein, 10 aa, Artificial Sequence
  Description of Artificial Sequence Synthetic peptide
  VHH12 CDR2 (AbM)
SITWNGGSTS

SEQ ID NO: 214 - protein, 13 aa, Artificial Sequence
  Description of Artificial Sequence Synthetic peptide
  VHH1 CDR3 (Exemplary)
GSIDLNWYGGMDY

SEQ ID NO: 215 - protein, 15 aa, Artificial Sequence
  Description of Artificial Sequence Synthetic peptide
  VHH2 CDR3 (Exemplary)
TTVLTDPRVLNEYAT

SEQ ID NO: 216 - protein, 12 aa, Artificial Sequence
  Description of Artificial Sequence Synthetic peptide
  VHH3 CDR3 (Exemplary)
DVFGSSGYVETY

SEQ ID NO: 217 - protein, 5 aa, Artificial Sequence
  Description of Artificial Sequence Synthetic peptide
  VHH4 CDR3 (Exemplary)
PLTAR

SEQ ID NO: 218 - protein, 7 aa, Artificial Sequence
  Description of Artificial Sequence Synthetic peptide
  VHH5 CDR3 (Exemplary)
DPFNQGY

SEQ ID NO: 219 - protein, 5 aa, Artificial Sequence
  Description of Artificial Sequence Synthetic peptide
  VHH6 CDR3 (Exemplary)
PLTSR

SEQ ID NO: 220 - protein, 22 aa, Artificial Sequence
  Description of Artificial Sequence Synthetic peptide
  VHH7 CDR3 (Exemplary)
MVNPIITAWGTIGVREIPDYDY

SEQ ID NO: 221 - protein, 4 aa, Artificial Sequence
  Description of Artificial Sequence Synthetic peptide
  VHH9 CDR3 (Exemplary)
QRGY

SEQ ID NO: 222 - protein, 7 aa, Artificial Sequence
  Description of Artificial Sequence Synthetic peptide
  VHH10 CDR3 (Exemplary)
DPFNQGY

SEQ ID NO: 223 - protein, 21 aa, Artificial Sequence
  Description of Artificial Sequence Synthetic peptide
  VHH11 CDR3 (Exemplary)
DLAEYSGTYSSPADSPAGYDY

SEQ ID NO: 224 - protein, 14 aa, Artificial Sequence
  Description of Artificial Sequence Synthetic peptide
  VHH12 CDR3 (Exemplary)
ARYYVSGTYFPANY

SEQ ID NO: 225 - protein, 14 aa, Artificial Sequence
  Description of Artificial Sequence Synthetic peptide
  VHH1 CDR3 (Contact)
AAGSIDLNWYGGMD

SEQ ID NO: 226 - protein, 16 aa, Artificial Sequence
  Description of Artificial Sequence Synthetic peptide
  VHH2 CDR3 (Contact)
AATTVLTDPRVLNEYA

SEQ ID NO: 227 - protein, 13 aa, Artificial Sequence
  Description of Artificial Sequence Synthetic peptide
  VHH3 CDR3 (Contact)
KADVFGSSGYVET

SEQ ID NO: 228 - protein, 6 aa, Artificial Sequence
  Description of Artificial Sequence Synthetic peptide
  VHH4 CDR3 (Contact)
NHPLTA

SEQ ID NO: 229 - protein, 8 aa, Artificial Sequence
  Description of Artificial Sequence Synthetic peptide
  VHH5 CDR3 (Contact)
AADPFNQG

SEQ ID NO: 230 - protein, 6 aa, Artificial Sequence
  Description of Artificial Sequence Synthetic peptide
  VHH6 CDR3 (Contact)
NHPLTS

SEQ ID NO: 231 - protein, 23 aa, Artificial Sequence
  Description of Artificial Sequence Synthetic peptide
  VHH7 CDR3 (Contact)
ASMVNPIITAWGTIGVREIPDYD

SEQ ID NO: 232 - protein, 5 aa, Artificial Sequence
  Description of Artificial Sequence Synthetic peptide
  VHH9 CDR3 (Contact)
NDQRG

SEQ ID NO: 233 - protein, 8 aa, Artificial Sequence
  Description of Artificial Sequence Synthetic peptide
  VHH10 CDR3 (Contact)
AADPFNQG

SEQ ID NO: 234 - protein, 22 aa, Artificial Sequence
  Description of Artificial Sequence Synthetic peptide
  VHH11 CDR3 (Contact)
AADLAEYSGTYSSPADSPAGYD

SEQ ID NO: 235 - protein, 15 aa, Artificial Sequence
  Description of Artificial Sequence Synthetic peptide
  VHH12 CDR3 (Contact)
AAARYYVSGTYFPAN

SEQ ID NO: 236 - protein, 13 aa, Artificial Sequence
  Description of Artificial Sequence Synthetic peptide
  VHH1 CDR3 (AbM)
GSIDLNWYGGMDY

SEQ ID NO: 237 - protein, 15 aa, Artificial Sequence
  Description of Artificial Sequence Synthetic peptide
  VHH2 CDR3 (AbM)
TTVLTDPRVLNEYAT

SEQ ID NO: 238 - protein, 12 aa, Artificial Sequence
  Description of Artificial Sequence Synthetic peptide
  VHH3 CDR3 (AbM)
DVFGSSGYVETY

SEQ ID NO: 239 - protein, 5 aa, Artificial Sequence
  Description of Artificial Sequence Synthetic peptide
  VHH4 CDR3 (AbM)
PLTAR

SEQ ID NO: 240 - protein, 7 aa, Artificial Sequence
  Description of Artificial Sequence Synthetic peptide
  VHH5 CDR3 (AbM)
DPFNQGY

SEQ ID NO: 241 - protein, 5 aa, Artificial Sequence
  Description of Artificial Sequence Synthetic peptide
  VHH6 CDR3 (AbM)
PLTSR

SEQ ID NO: 242 - protein, 22 aa, Artificial Sequence
  Description of Artificial Sequence Synthetic peptide
  VHH7 CDR3 (AbM)
MVNPIITAWGTIGVREIPDYDY

SEQ ID NO: 243 - protein, 4 aa, Artificial Sequence
  Description of Artificial Sequence Synthetic peptide
  VHH9 CDR3 (AbM)
QRGY

SEQ ID NO: 244 - protein, 7 aa, Artificial Sequence
  Description of Artificial Sequence Synthetic peptide
  VHH10 CDR3 (AbM)
DPFNQGY

SEQ ID NO: 245 - protein, 21 aa, Artificial Sequence
  Description of Artificial Sequence Synthetic peptide
  VHH11 CDR3 (AbM)
DLAEYSGTYSSPADSPAGYDY

SEQ ID NO: 246 - protein, 14 aa, Artificial Sequence
  Description of Artificial Sequence Synthetic peptide
  VHH12 CDR3 (AbM)
ARYYVSGTYFPANY

SEQ ID NO: 247 - protein, 113 aa, Homo sapiens
  hpIgR_073
  MOD_RES (96)..(96): Any amino acid
  MOD_RES (99)..(99): Any amino acid
EVQVVESGGGLVQAGGSLRLSCAVSGTSVSSNAMGWYRQAPGKQREWVGFIDRIATTTIATSVKGRFAITRDNAKNTVYLQMSGLKPEDTAVYYCXHPXTARWGQGTQVTVSS

SEQ ID NO: 248 - protein, 130 aa, Homo sapiens
  hpIgR_201
QVQLVESGGGLVQPGGSLRLSCAASGRPNSKYAMAWFRRAPGKEREFQAAINWSGANTYYGDSVKGRFTISRDNAKNTVTLQMNNLNPEDTAVYYCAADNRAYTYHTFDYYQTDASYVYWGQGTQVTVSS

SEQ ID NO: 249 - protein, 127 aa, Mus sp.
  mpIgR_349
QLQLVESGGGLVQAGGSLRLSCAASGRTFSTSTMGWFRQAPGKEREFVAAIQWSSASASTYYYYADSVKGRFTISRDNARNTVSLQMNSLKPEDTAVYYCANLVFRVGALKERDDYWGQGTQVTVSS

SEQ ID NO: 250 - protein, 110 aa, Homo sapiens
  hpIgR_D1
KSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVS

SEQ ID NO: 251 - protein, 110 aa, Mus sp.
  mpIgR_D1
KSPIFGPQEVSSIEGDSVSITCYYPDTSVNRHTRKYWCRQGASGMCTTLISSNGYLSKEYSGRANLINFPENNTFVINIEQLTQDDTGSYKCGLGTSNRGLSFDVSLEVS

SEQ ID NO: 252 - protein, 110 aa, Artificial Sequence
  Description of Artificial Sequence Synthetic polypeptide
  Consensus
  MOD_RES (8)..(8): Glu or Gln
  MOD_RES (11)..(11): Asn or Ser
  MOD_RES (16)..(16): Asn or Asp
  MOD_RES (26)..(26): Pro or Asp
  MOD_RES (43)..(43): Arg or Ser
  MOD_RES (45)..(45): Gly or Met
  MOD_RES (47)..(47): Ile or Thr
  MOD_RES (53)..(53): Glu or Asn
  MOD_RES (58)..(58): Ser or Lys
  MOD_RES (59)..(59): Lys or Glu
  MOD_RES (67)..(67): Thr or Ile
  MOD_RES (73)..(73): Gly or Asn
  MOD_RES (80)..(80): Ala or Glu
  MOD_RES (89)..(89): Arg or Ser
  MOD_RES (96)..(96): Ile or Thr
  MOD_RES (97)..(97): Asn or Ser
  MOD_RES (98)..(98): Ser or Asn
KSPIFGPXEVXSIEGXSVSITCYYPXTSVNRHTRKYWCRQGAXGXCXTLISSXGYLSXXYAGRANLXNFPENXTFVINIXQLSQDDSGXYKCGLGXXXRGLSFDVSLEVS

SEQ ID NO: 253 - protein, 113 aa, Oncorhynchus mykiss
  tpIgR_D1
RVTTVGDLAVLEGRSVMIPCHYGPQYASYVKYWCRGSVKDLCTSLVRSDAPRGPAAAGEDKVVMFDDPVQQVFTVTMTELQKEDSGWYWCGVEVGGVWSADVTASLHINVIQG

SEQ ID NO: 254 - protein, 109 aa, Homo sapiens
  hpIgR_D1
KSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEV

SEQ ID NO: 255 - protein, 107 aa, Artificial Sequence
  Description of Artificial Sequence Synthetic polypeptide
  hD1_tCDR1
KSPIFGPEEVNSVEGNSVSITCYYGPQYASYRKYWCRQGARGGCITLISSEGYVSSKYAGRANLTNFPENGTFVVNIAQLSQDDSGRYKCGLGINSRGLSFDVSLEV

SEQ ID NO: 256 - protein, 109 aa, Artificial Sequence
  Description of Artificial Sequence Synthetic polypeptide
  hD1_tCDR2
KSPIFGPEEVNSVEGNSVSITCYYPPTSVNRHTRKYWCRQGARGGCITLISSDAPVSSKYAGRANLTNFPENGTFVVNIAQLSQDDSGRYKCGLGINSRGLSFDVSLEV

SEQ ID NO: 257 - protein, 112 aa, Artificial Sequence
  Description of Artificial Sequence Synthetic polypeptide
  hD1_tCDR3
KSPIFGPEEVNSVEGNSVSITCYYPPTSVNRHTRKYWCRQGARGGCITLISSEGYVSSKYAGRANLTNFPENGTFVVNIAQLSQDDSGRYKCGLGVGGVWSADLSFDVSLEV

SEQ ID NO: 258 - protein, 7 aa, Artificial Sequence
  Description of Artificial Sequence Synthetic peptide
  VHH11 CDR1 (Kabat)
FTTYRMG

SEQ ID NO: 259 - protein, 5 aa, Artificial Sequence
  Description of Artificial Sequence Synthetic peptide
  VHH11 CDR1 (Kabat) - alternative
TYRMG

SEQ ID NO: 260 - protein, 7 aa, Artificial Sequence
  Description of Artificial Sequence Synthetic peptide
  VHH1 and VHH2 CDR2 (Chothia) - alternative
WNGRGTY

SEQ ID NO: 261 - protein, 3 aa, Artificial Sequence
  Description of Artificial Sequence Synthetic peptide
  VHH3 CDR2 (Chothia) - alternative
GGG

SEQ ID NO: 262 - protein, 3 aa, Artificial Sequence
  Description of Artificial Sequence Synthetic peptide
  VHH4 CDR2 (Chothia) - alternative
RIA

SEQ ID NO: 263 - protein, 4 aa, Artificial Sequence
  Description of Artificial Sequence Synthetic peptide
  VHH5 CDR2 (Chothia) - alternative
WNGG

SEQ ID NO: 264 - protein, 3 aa, Artificial Sequence
  Description of Artificial Sequence Synthetic peptide
  VHH6 CDR2 (Chothia) - alternative
GGG

SEQ ID NO: 265 - protein, 3 aa, Artificial Sequence
  Description of Artificial Sequence Synthetic peptide
  VHH7 CDR2 (Chothia) - alternative
GGG

SEQ ID NO: 266 - protein, 4 aa, Artificial Sequence
  Description of Artificial Sequence Synthetic peptide
  VHH9 CDR2 (Chothia) - alternative
WSGG

SEQ ID NO: 267 - protein, 4 aa, Artificial Sequence
  Description of Artificial Sequence Synthetic peptide
  VHH10 CDR2 (Chothia) - alternative
WSGS

SEQ ID NO: 268 - protein, 4 aa, Artificial Sequence
  Description of Artificial Sequence Synthetic peptide
  VHH11 CDR2 (Chothia) - alternative
WSGG

SEQ ID NO: 269 - protein, 4 aa, Artificial Sequence
  Description of Artificial Sequence Synthetic peptide
  VHH12 CDR2 (Chothia) - alternative
WNGG

SEQ ID NO: 270 - protein, 11 aa, Artificial Sequence
  Description of Artificial Sequence Synthetic peptide
  VHH1 and VHH2 CDR2 (IMGT) - alternative
IDWNGRGTYYR

SEQ ID NO: 271 - protein, 4 aa, Artificial Sequence
  Description of Artificial Sequence Synthetic peptide
  VHH9 CDR3 (Kabat) - alternative
QRGY

SEQ ID NO: 272 - protein, 11 aa, Artificial Sequence
  Description of Artificial Sequence Synthetic peptide
  VHH1 CDR3 (Chothia) - alternative
SIDLNWYGGMD

SEQ ID NO: 273 - protein, 13 aa, Artificial Sequence
  Description of Artificial Sequence Synthetic peptide
  VHH2 CDR3 (Chothia) - alternative
TVLTDPRVLNEYA

SEQ ID NO: 274 - protein, 10 aa, Artificial Sequence
  Description of Artificial Sequence Synthetic peptide
  VHH3 CDR3 (Chothia) - alternative
VFGSSGYVET

SEQ ID NO: 275 - protein, 3 aa, Artificial Sequence
  Description of Artificial Sequence Synthetic peptide
  VHH4 CDR3 (Chothia) - alternative
LTA

SEQ ID NO: 276 - protein, 5 aa, Artificial Sequence
  Description of Artificial Sequence Synthetic peptide
  VHH5 CDR3 (Chothia) - alternative
PFNQG

SEQ ID NO: 277 - protein, 3 aa, Artificial Sequence
  Description of Artificial Sequence Synthetic peptide
  VHH6 CDR3 (Chothia) - alternative
LTS

SEQ ID NO: 278 - protein, 20 aa, Artificial Sequence
  Description of Artificial Sequence Synthetic peptide
  VHH7 CDR3 (Chothia) - alternative
VNPIITAWGTIGVREIPDYD

SEQ ID NO: 279 - protein, 2 aa, Artificial Sequence
  Description of Artificial Sequence Synthetic peptide
  VHH9 CDR3 (Chothia) - alternative
RG

SEQ ID NO: 280 - protein, 5 aa, Artificial Sequence
  Description of Artificial Sequence Synthetic peptide
  VHH10 CDR3 (Chothia) - alternative
PFNQG

SEQ ID NO: 281 - protein, 19 aa, Artificial Sequence
  Description of Artificial Sequence Synthetic peptide
  VHH11 CDR3 (Chothia) - alternative
LAEYSGTYSSPADSPAGYD

SEQ ID NO: 282 - protein, 12 aa, Artificial Sequence
  Description of Artificial Sequence Synthetic peptide
  VHH12 CDR3 (Chothia) - alternative
RYYVSGTYFPAN

SEQ ID NO: 283 - protein, 15 aa, Artificial Sequence
  Description of Artificial Sequence Synthetic peptide
  VHH1 CDR3 (IMGT) - alternative
AAGSIDLNWYGGMDY

SEQ ID NO: 284 - protein, 17 aa, Artificial Sequence
  Description of Artificial Sequence Synthetic peptide
  VHH2 CDR3 (IMGT) - alternative
AATTVLTDPRVLNEYAT

SEQ ID NO: 285 - protein, 100 aa, Artificial Sequence
  Germline Sequence 1
QVQLVESGGGLVQAGGSLKLACAAPGLTFSSYRMGWFRQAPGQEREFVAAIDWNGRGTYYRYYADSVKGRSTISRDNAKNTMYLQMNSLKPEDTAVYYCA

SEQ ID NO: 286 - protein, 100 aa, Artificial Sequence
  Germline Sequence 2
EVQVVESGGGLVQAGGSLKLACAAPGLTFSSYRMGWFRQAPGQEREFVAAIDWNGRGTYYRYYADSVKGRSTISRDNAKNTVYLQMNSLKPEDTAVYYCA

SEQ ID NO: 287 - protein, 97 aa, Artificial Sequence
  Germline Sequence 3
QLQLVESGGGLVQPGGSLRLSCAASGSIFSINVMGWYRQAPGKQRELVARINGGGITHYAESVKGRFTISRDNAKNTVYLQMNSLKPEDTAAYYCKA

SEQ ID NO: 288 - protein, 96 aa, Artificial Sequence
  Germline Sequence 4
EVQVVESGGGLVQAGGSLRLSCAVSGTSVSSNAMGWYRQAPGKQREWVGFIDRIATTTIATSVKGRFAITRDNAKNTVYLQMSGLKPEDTAVYYCN

SEQ ID NO: 289 - protein, 98 aa, Artificial Sequence
  Germline Sequence 5
QVQLVESGGGLVQAGGSLRLSCAASGRTFSSYAMGWFRQAPGKEREFVAAITWNGGTTYYADSVKGRFTISRDNAKNTVYLQMNSLKPEDTAVYYCAA

SEQ ID NO: 290 - protein, 96 aa, Artificial Sequence
  Germline Sequence 6
EVQLVESGGGLVQAGGSLRLSCAVSGSSVSSDAMGWYRQAPGNQRAWVAFISGGGTTTYADSVKGRFTISRDNTKNTVYLHMNSLKPEDTAVYYCN

SEQ ID NO: 291 - protein, 95 aa, Artificial Sequence
  Germline Sequence 7
EVQVVESGGGLVQAGGSLRLACVASRSIGSINVMGWYRQAPGKQRDLVARITGGGSTHYAESVKGRFTISRDNAKNTVYLQMNSLEPEDTAVYYC

SEQ ID NO: 292 - protein, 97 aa, Artificial Sequence
  Germline Sequence 8
QVQLVESGGGLVQAGGSLRLSCAVSGRTFSTYRMGWFRQAPGKERSFVAAISWSGGSTTYADPVKGRFTISRDNAKNTVYLRMNSLKPEDTAVYYCN

SEQ ID NO: 293 - protein, 97 aa, Artificial Sequence
  Germline Sequence 9
EVQVVESGGGLVQAGGSLRLSCAASGFTFTRYAMGWFRQAPGKERSFVAAISWSGSSAGYGDSVKGRFTISRDNAKNTLYLQMNSLKPEDTAVYYCA

SEQ ID NO: 294 - protein, 98 aa, Artificial Sequence
  Germline Sequence 10
EVQVVESGGGLVQAGGSLRLSCAASGRTFTTYRMGWFRQAPGKEREFVAAIRWSGGRTLYADSVKGRFTISRDNAKNTAYLQMNNLRPEDTAVYYCAA

SEQ ID NO: 295 - protein, 100 aa, Artificial Sequence
  Germline Sequence 11
QVQLVETGGGLVQAGDSLRLSCAASGRTLSFNTYAMGWFRQAPGKEREFVASITWNGGSTSYADSVKGRFTITRDNAKNTATLRMNSLQPDDTAVYYCAA

SEQ ID NO: 296 - protein, 13 aa, Artificial Sequence
  J Segment 1
DYWGQGTQVTVSS

SEQ ID NO: 297 - protein, 15 aa, Artificial Sequence
  J Segment 2
EYATWGQGTQVTVSS

SEQ ID NO: 298 - protein, 12 aa, Artificial Sequence
  J Segment 3
YWGQGTQVTVSS

SEQ ID NO: 299 - protein, 11 aa, Artificial Sequence
  J Segment 4
WGQGTQVTVSS

SEQ ID NO: 300 - protein, 15 aa, Artificial Sequence
  J Segment 5
DYDYWGQGTQVTVSS

SEQ ID NO: 301 - protein, 11 aa, Artificial Sequence
  J Segment 6
WGQGTLVTVSS

SEQ ID NO: 302 - protein, 14 aa, Artificial Sequence
  J Segment 7
YDYWGQGTQVTVSS

SEQ ID NO: 303 - protein, 13 aa, Artificial Sequence
  J Segment 8
NYWGQGTQVTVSS

(19) United States
(12) Patent Application Publication
(10) Pub. No.: US 2023/0019640 A1
(43) Pub. Date: Jan. 19, 2023

(54) MATERIALS AND METHODS FOR POLYMERIC ANTIBODY RECEPTOR TARGETING

(71) Applicant: JANSSEN BIOTECH, INC., Horsham, PA (US)

(72) Inventors: Rajkumar GANESAN, Blue Bell, PA (US); Bharathikumar Vellalore MARUTHACHALAM, Downington, PA (US); Adam ZWOLAK, Bala Cynwyd, PA (US); Brian GEIST, West Chester, PA (US); Xiefan LIN-SCHMIDT, Ambler, PA (US); Sathyadevi VENKATARAMANI, Blue Bell, PA (US); Sanjaya SINGH, Blue Bell, PA (US)

(73) Assignee: JANSSEN BIOTECH, INC., Horsham, PA (US)

(21) Appl. No.: 17/630,706

(22) PCT Filed: Jul. 31, 2020
(86) PCT No.: PCT/US2020/044505

§ 371 Date: Jan. 27, 2022

Related U.S. Application Data
(60) Provisional application No. 62/940,228, filed on Nov. 25, 2019.
(60) Provisional application No. 62/940,232, filed on Nov. 25, 2019.
(60) Provisional application No. 62/940,220, filed on Nov. 25, 2019.
(60) Provisional application No. 62/940,208, filed on Nov. 25, 2019.
(60) Provisional application No. 62/940,206, filed on Nov. 25, 2019.
(60) Provisional application No. 62/940,200, filed on Nov. 25, 2019.
(60) Provisional application No. 62/940,196, filed on Nov. 25, 2019.
(60) Provisional application No. 62/882,387, filed on Aug. 2, 2019.
(60) Provisional application No. 62/882,361, filed on Aug. 2, 2019.
(60) Provisional application No. 62/882,346, filed on Aug. 2, 2019.
(60) Provisional application No. 62/882,291, filed on Aug. 2, 2019.

(51) Int. Cl. C07K 16/28; A61K 47/68; A61P 11/00

(52) U.S. Cl. CPC C07K 16/283; A61K 47/6849; A61P 11/00; C07K 2317/569; C07K 2317/92; C07K 2317/567; A61K 2039/544

(57) ABSTRACT

VHH domains that bind to p1gR are described. The VHH domain may compete with IgA binding to p1gR, or alternatively, the VHH domain may compete with IgA binding to p1gR. The VHH domains may be coupled to therapeutic agents so as to facilitate delivery of the therapeutic agent to the mucosal layer via p1gR-mediated transcytosis. The therapeutic agent can be a small molecule, large molecule, or even an antibody.

### CROSS-REFERENCE TO RELATED APPLICATIONS

This application claims the benefit of U.S. Provisional Patent Application No. 62/940,232, filed Nov. 25, 2019, U.S. Provisional Patent Application No. 62/940,228, filed Nov. 25, 2019, U.S. Provisional Patent Application No. 62/940,220, filed Nov. 25, 2019, U.S. Provisional Patent Application No. 62/940,208, filed Nov. 25, 2019, U.S. Provisional Patent Application No. 62/940,206, filed Nov. 25, 2019, U.S. Provisional Patent Application No. 62/940,200, filed Nov. 25, 2019, U.S. Provisional Patent Application No. 62/940,196, filed Nov. 25, 2019, U.S. Provisional Patent Application No. 62/882,387, filed Aug. 2, 2019, U.S. Provisional Patent Application No. 62/882,361, filed Aug. 2, 2019, U.S. Provisional Patent Application No. 62/882,346, filed Aug. 2, 2019 and U.S. Provisional Patent Application No. 62/882,291, filed Aug. 2, 2019, each of which is incorporated by reference herein in its entirety.

### SEQUENCE LISTING

This application incorporates by reference a Sequence Listing submitted with this application as a text format, entitled “14620-203-228_SL.txt”, created on Jul. 29, 2020 having a size of 181,728 bytes.

### 1. TECHNICAL FIELD

This invention relates to materials and methods for delivery of agents to, into and across mucosal epithelial cells. The materials and methods may be effective to deliver agents, including small molecules and proteins, such as antibodies or fragments thereof, from systemic circulation to the mucosa or epithelial cells. The materials and methods may also be effective to deliver agents, including peptides, antibodies or fragments thereof, and vaccines to systemic circulation or Lamina propria.

### 2. BACKGROUND

Targeted delivery of diagnostics and therapeutics can overcome several issues in drug delivery, such as systemic toxicity, circulation, cell barriers, bioavailability, targeted and controlled release, PK and clearance. Targeted delivery of molecules to highly compartmentalized organs by preferred routes of administration may be highly beneficial.

The human mucosa forms an elaborate extracellular environment, in which the immune system mediates host interactions with commensal and pathogenic agents. Mucosal protection is largely conferred through the function of polymeric immunoglobulin receptor (pIgR), the oldest identifiable Fc receptor. pIgR transports soluble polymeric forms of IgA and IgM into apical mucosal tissues from the basolateral side of the epithelium. pIgR expression is under the strong regulation of cytokines, hormones and pathogenic stimuli. It is upregulated during infection and inflammation.

### 3. SUMMARY

In one aspect is provided a VHH domain that binds to an extracellular domain of pIgR. In some embodiments, the VHH domain binds to an extracellular domain 1 of pIgR. In some embodiments, the VHH domain binds to an extracellular domain 2 of pIgR. In some embodiments, the VHH domain binds to an extracellular domain 1-2 of pIgR. In some embodiments, the VHH domain binds to an extracellular domain 3 of pIgR. In some embodiments, the VHH domain binds to an extracellular domain 2-3 of pIgR. In some embodiments, the VHH domain binds to an extracellular domain 4-5 of pIgR. In some embodiments, the VHH domain binds to an extracellular domain 5 of pIgR. In some embodiments, the pIgR is human pIgR. In some embodiments, the pIgR is mouse pIgR. In some embodiments, the VHH domain does not detectably bind to the amino acid sequence of EKAVADTRDQADGSRASVDSGSSEEQGGSSR (SEQ ID NO: 143), EREIQNVGDQAQENRASGDAGSADGQSRSSSSK (SEQ ID NO: 144) or EREIQNVRDQAQENRASGDAGSADGQSRSSSSK (SEQ ID NO: 145). In some embodiments, the VHH domain competes with IgA binding to the pIgR. In some embodiments, the VHH domain promotes IgA binding to the pIgR. In some embodiments, the K_{D} of the binding of the VHH domain to pIgR is from about 4 to about 525 nM. In some embodiments, the K_{D} of the binding of the VHH domain to pIgR is less than about 50 nM. In some embodiments, the K_{D} of the binding of the VHH domain to pIgR is from about 4 to about 34 nM. In some embodiments, the Tₘ of the VHH domain is from about 53 to about 77° C. In some embodiments, the Tₘ of the VHH domain is from 53.9 to 76.4° C.

In some embodiments, the VHH domain comprises a CDR1 sequence of SYRMG (SEQ ID NO: 1), INVMG (SEQ ID NO: 2), SNAMG (SEQ ID NO: 3), SYAMG (SEQ ID NO: 4), SDAMG (SEQ ID NO: 5), INVMG (SEQ ID NO: 6), TYRMG (SEQ ID NO: 7), RYAMG (SEQ ID NO: 8), FTTYRMG (SEQ ID NO: 258), TYRMG (SEQ ID NO: 259), FNTYAMG (SEQ ID NO: 9), GLTFSSY (SEQ ID NO: 10), GSIFSIN (SEQ ID NO: 11), GTSVSSN (SEQ ID NO: 12), GRTFSSY (SEQ ID NO: 13), GSSVSSD (SEQ ID NO: 14), RSIGSIN (SEQ ID NO: 15), GRTFSTY (SEQ ID NO: 16), GFTFTRY (SEQ ID NO: 17), GRTFTTY (SEQ ID NO: 18), GRTLSFNTY (SEQ ID NO: 19), GLTFSSYR (SEQ ID NO: 20), GSIFSINV (SEQ ID NO: 21), GTSVSSNA (SEQ ID NO: 22), GRTFSSYA (SEQ ID NO: 23), GSSVSSDA (SEQ ID NO: 24), RSIGSINV (SEQ ID NO: 25), GRTFSTYR (SEQ ID NO: 26), GFTFTRYA (SEQ ID NO: 27), GRTFTTYR (SEQ ID NO: 28), GRTLSFNTYA (SEQ ID NO: 29), GLTFSSYRMG (SEQ ID NO: 154), GSIFSINVMG (SEQ ID NO: 155), GTSVSSNAMG (SEQ ID NO: 156), GRTFSSYAMG (SEQ ID NO: 157), GSSVSSDAMG (SEQ ID NO: 158), RSIGSINVMG (SEQ ID NO: 159), GRTFSTYRMG (SEQ ID NO: 160), GFTFTRYAMG (SEQ ID NO: 161), GRTFTTYRMG (SEQ ID NO: 162), GRTLSFNTYAMG (SEQ ID NO: 163), SSYRMG (SEQ ID NO: 164), SINVMG (SEQ ID NO: 165), SSNAMG (SEQ ID NO: 166), SSYAMG (SEQ ID NO: 167), SSDAMG (SEQ ID NO: 168), SINVMG (SEQ ID NO: 169), STYRMG (SEQ ID NO: 170), TRYAMG (SEQ ID NO: 171), TTYRMG (SEQ ID NO: 172), SFNTYAMG (SEQ ID NO: 173), GLTFSSYRMG (SEQ ID NO: 174), GSIFSINVMG (SEQ ID NO: 175), GTSVSSNAMG (SEQ ID NO: 176), GRTFSSYAMG (SEQ ID NO: 177), GSSVSSDAMG (SEQ ID NO: 178), RSIGSINVMG (SEQ ID NO: 179), GRTFSTYRMG (SEQ ID NO: 180), GFTFTRYAMG (SEQ ID NO: 181), GRTFTTYRMG (SEQ ID NO: 182), or GRTLSFNTYAMG (SEQ ID NO: 183).

In some embodiments, the VHH domain comprises a CDR2 sequence of

(SEQ ID NO: 30)
AIDWNGRGTYYRYYADSVKG,
(SEQ ID NO: 31)
RINGGGITHYAESVKG,
(SEQ ID NO: 32)
FIDRIATTTIATSVKG,
(SEQ ID NO: 33)
AITWNGGTTYYADSVKG,
(SEQ ID NO: 34)
FISGGGTTTYADSVKG,
(SEQ ID NO: 35)
RITGGGSTHYAESVKG,
(SEQ ID NO: 36)
AISWSGGSTTYADPVKG,
(SEQ ID NO: 37)
AISWSGSSAGYGDSVKG,
(SEQ ID NO: 38)
AIRWSGGRTLYADSVKG,
(SEQ ID NO: 39)
SITWNGGSTSYADSVKG,
(SEQ ID NO: 40)
DWNGRGTYY,
(SEQ ID NO: 260)
WNGRGTY,
(SEQ ID NO: 41)
NGGGI,
(SEQ ID NO: 261)
GGG,
(SEQ ID NO: 42)
DRIAT,
(SEQ ID NO: 262)
RIA,
(SEQ ID NO: 43)
TWNGGT,
(SEQ ID NO: 263)
WNGG,
(SEQ ID NO: 44)
SGGGT,
(SEQ ID NO: 264)
GGG,
(SEQ ID NO: 45)
TGGGS,
(SEQ ID NO: 265)
GGG,
(SEQ ID NO: 46)
SWSGGS,
(SEQ ID NO: 266)
WSGG,
(SEQ ID NO: 47)
SWSGSS,
(SEQ ID NO: 267)
WSGS,
(SEQ ID NO: 48)
RWSGGR,
(SEQ ID NO: 268)
WSGG,
(SEQ ID NO: 49)
TWNGGS,
(SEQ ID NO: 269)
WNGG,
(SEQ ID NO: 50)
IDWNGRGTYY,
(SEQ ID NO: 270)
IDWNGRGTYYR,
(SEQ ID NO: 51)
INGGGIT,
(SEQ ID NO: 52)
IDRIATT,
(SEQ ID NO: 53)
ITWNGGTT,
(SEQ ID NO: 54)
ISGGGTT,
(SEQ ID NO: 55)
ITGGGST,
(SEQ ID NO: 56)
ISWSGGST,
(SEQ ID NO: 57)
ISWSGSSA,
(SEQ ID NO: 58)
IRWSGGRT,
(SEQ ID NO: 59)
ITWNGGST,
(SEQ ID NO: 184)
AIDWNGRGTYYRYYADSVKG,
(SEQ ID NO: 185)
RINGGGITHYAESVKG,
(SEQ ID NO: 186)
FIDRIATTTIATSVKG,
(SEQ ID NO: 187)
AITWNGGTTYYADSVKG,
(SEQ ID NO: 188)
FISGGGTTTYADSVKG,
(SEQ ID NO: 189)
RITGGGSTHYAESVKG,
(SEQ ID NO: 190)
AISWSGGSTTYADPVKG,
(SEQ ID NO: 191)
AISWSGSSAGYGDSVKG,
(SEQ ID NO: 192)
AIRWSGGRTLYADSVKG,
(SEQ ID NO: 193)
SITWNGGSTSYADSVKG,
(SEQ ID NO: 194)
FVAAIDWNGRGTYYRY,
(SEQ ID NO: 195)
LVARINGGGITH,
(SEQ ID NO: 196)
WVGFIDRIATTT,
(SEQ ID NO: 197)
FVAAITWNGGTTY,
(SEQ ID NO: 198)
WVAFISGGGTTT,
(SEQ ID NO: 199)
LVARITGGGSTH,
(SEQ ID NO: 200)
FVAAISWSGGSTT,
(SEQ ID NO: 201)
FVAAISWSGSSAG,
(SEQ ID NO: 202)
FVAAIRWSGGRTL,
(SEQ ID NO: 203)
FVASITWNGGSTS,
(SEQ ID NO: 204)
AIDWNGRGTYYRY,
(SEQ ID NO: 205)
RINGGGITH,
(SEQ ID NO: 206)
FIDRIATTT,
(SEQ ID NO: 207)
AITWNGGTTY,
(SEQ ID NO: 208)
FISGGGTTT,
(SEQ ID NO: 209)
RITGGGSTH,
(SEQ ID NO: 210)
AISWSGGSTT,
(SEQ ID NO: 211)
AISWSGSSAG,
(SEQ ID NO: 212)
AIRWSGGRTL,
or
(SEQ ID NO: 213)
SITWNGGSTS.

In some embodiments, the VHH domain comprises a CDR3 sequence of

(SEQ ID NO: 60)
GSIDLNWYGGMDY,
(SEQ ID NO: 61)
TTVLTDPRVLNEYAT,
(SEQ ID NO: 62)
DVFGSSGYVETY,
(SEQ ID NO: 63)
PLTAR,
(SEQ ID NO: 64)
DPFNQGY,
(SEQ ID NO: 65)
PLTSR,
(SEQ ID NO: 66)
MVNPIITAWGTIGVREIPDYDY,
(SEQ ID NO: 67)
DQRGY,
(SEQ ID NO: 271)
QRGY,
(SEQ ID NO: 68)
DPFNQGY,
(SEQ ID NO: 69)
DLAEYSGTYSSPADSPAGYDY,
(SEQ ID NO: 70)
ARYYVSGTYFPANY,
(SEQ ID NO: 71)
GSIDLNWYGGMDY,
(SEQ ID NO: 272)
SIDLNWYGGMD,
(SEQ ID NO: 72)
TTVLTDPRVLNEYAT,
(SEQ ID NO: 273)
TVLTDPRVLNEYA,
(SEQ ID NO: 73)
DVFGSSGYVETY,
(SEQ ID NO: 274)
VFGSSGYVET,
(SEQ ID NO: 74)
PLTAR,
(SEQ ID NO: 275)
LTA,
(SEQ ID NO: 75)
DPFNQGY,
(SEQ ID NO: 276)
PFNQG,
(SEQ ID NO: 76)
PLTSR,
(SEQ ID NO: 277)
LTS,
(SEQ ID NO: 77)
MVNPIITAWGTIGVREIPDYDY,
(SEQ ID NO: 278)
VNPIITAWGTIGVREIPDYD,
(SEQ ID NO: 78)
DQRGY,
(SEQ ID NO: 279)
RG,
(SEQ ID NO: 79)
DPFNQGY,
(SEQ ID NO: 280)
PFNQG,
(SEQ ID NO: 80)
DLAEYSGTYSSPADSPAGYDY,
(SEQ ID NO: 281)
LAEYSGTYSSPADSPAGYD,
(SEQ ID NO: 81)
ARYYVSGTYFPANY,
(SEQ ID NO: 282)
RYYVSGTYFPAN,
(SEQ ID NO: 82)
CAAGSIDLNWYGGMDY,
(SEQ ID NO: 283)
AAGSIDLNWYGGMDY,
(SEQ ID NO: 83)
CAATTVLTDPRVLNEYAT,
(SEQ ID NO: 284)
AATTVLTDPRVLNEYAT,
(SEQ ID NO: 84)
KADVFGSSGYVETY,
(SEQ ID NO: 85)
NHPLTAR,
(SEQ ID NO: 86)
AADPFNQGY,
(SEQ ID NO: 87)
NHPLTSR,
(SEQ ID NO: 88)
ASMVNPIITAWGTIGVREIPDYDY,
(SEQ ID NO: 89)
NDQRGY,
(SEQ ID NO: 90)
AADPFNQGY,
(SEQ ID NO: 91)
AADLAEYSGTYSSPADSPAGYDY,
(SEQ ID NO: 92)
AAARYYVSGTYFPANY,
(SEQ ID NO: 214)
GSIDLNWYGGMDY,
(SEQ ID NO: 215)
TTVLTDPRVLNEYAT,
(SEQ ID NO: 216)
DVFGSSGYVETY,
(SEQ ID NO: 217)
PLTAR,
(SEQ ID NO: 218)
DPFNQGY,
(SEQ ID NO: 219)
PLTSR,
(SEQ ID NO: 220)
MVNPIITAWGTIGVREIPDYDY,
(SEQ ID NO: 221)
QRGY,
(SEQ ID NO: 222)
DPFNQGY,
(SEQ ID NO: 223)
DLAEYSGTYSSPADSPAGYDY,
(SEQ ID NO: 224)
ARYYVSGTYFPANY,
(SEQ ID NO: 225)
AAGSIDLNWYGGMD,
(SEQ ID NO: 226)
AATTVLTDPRVLNEYA,
(SEQ ID NO: 227)
KADVFGSSGYVET,
(SEQ ID NO: 228)
NHPLTA,
(SEQ ID NO: 229)
AADPFNQG,
(SEQ ID NO: 230)
NHPLTS,
(SEQ ID NO: 231)
ASMVNPIITAWGTIGVREIPDYD,
(SEQ ID NO: 232)
NDQRG,
(SEQ ID NO: 233)
AADPFNQG,
(SEQ ID NO: 234)
AADLAEYSGTYSSPADSPAGYD,
(SEQ ID NO: 235)
AAARYYVSGTYFPAN,
(SEQ ID NO: 236)
GSIDLNWYGGMDY,
(SEQ ID NO: 237)
TTVLTDPRVLNEYAT,
(SEQ ID NO: 238)
DVFGSSGYVETY,
(SEQ ID NO: 239)
PLTAR,
(SEQ ID NO: 240)
DPFNQGY,
(SEQ ID NO: 241)
PLTSR,
(SEQ ID NO: 242)
MVNPIITAWGTIGVREIPDYDY,
(SEQ ID NO: 243)
QRGY,
(SEQ ID NO: 244)
DPFNQGY,
(SEQ ID NO: 245)
DLAEYSGTYSSPADSPAGYDY,
or
(SEQ ID NO: 246)
ARYYVSGTYFPANY.

In some embodiments, the VHH domain comprises a CDR1 sequence, a CDR2 sequence, and a CDR3 sequence present in VHH1. Accordingly, in some embodiments, the VHH domain comprises: i) the CDR1 sequence of SYRMG (SEQ ID NO: 1), the CDR2 sequence of AIDWNGRGTYYRYYADSVKG (SEQ ID NO: 30), and the CDR3 sequence of GSIDLNWYGGMDY (SEQ ID NO: 60); ii) the CDR1 sequence of GLTFSSY (SEQ ID NO: 10), the CDR2 sequence of DWNGRGTYY (SEQ ID NO: 40) or WNGRGTY (SEQ ID NO: 260), and the CDR3 sequence of GSIDLNWYGGMDY (SEQ ID NO: 71) or SIDLNWYGGMD (SEQ ID NO: 272); iii) the CDR1 sequence of GLTFSSYR (SEQ ID NO: 20), the CDR2 sequence of IDWNGRGTYY (SEQ ID NO: 50) or IDWNGRGTYYR (SEQ ID NO: 270), and the CDR3 sequence of CAAGSIDLNWYGGMDY (SEQ ID NO: 82) or AAGSIDLNWYGGMDY (SEQ ID NO: 283); iv) the CDR1 sequence of GLTFSSYRMG (SEQ ID NO: 154), the CDR 2 sequence of AIDWNGRGTYYRYYADSVKG (SEQ ID NO: 184), and the CDR3 sequence of GSIDLNWYGGMDY (SEQ ID NO: 214); v) the CDR1 sequence of SSYRMG (SEQ ID NO: 164), the CDR2 sequence of FVAAIDWNGRGTYYRY (SEQ ID NO: 194), and the CDR3 sequence of AAGSIDLNWYGGMD (SEQ ID NO: 225); or vi) the CDR1 sequence of GLTFSSYRMG (SEQ ID NO: 174), the CDR2 sequence of AIDWNGRGTYYRY (SEQ ID NO: 204), and the CDR3 sequence of GSIDLNWYGGMDY (SEQ ID NO: 236).

In some embodiments, the VHH domain comprises a CDR1 sequence, a CDR2 sequence, and a CDR3 sequence present in VHH2. Accordingly, in some embodiments, the VHH domain comprises: i) the CDR1 sequence of SYRMG (SEQ ID NO: 1), the CDR2 sequence of AIDWNGRGTYYRYYADSVKG (SEQ ID NO: 30), and the CDR3 sequence of TTVLTDPRVLNEYAT (SEQ ID NO: 61); ii) the CDR1 sequence of GLTFSSY (SEQ ID NO: 10), the CDR2 sequence of DWNGRGTYY (SEQ ID NO: 40) or WNGRGTY (SEQ ID NO: 260), and the CDR3 sequence of TTVLTDPRVLNEYAT (SEQ ID NO: 72) or TVLTDPRVLNEYA (SEQ ID NO: 273); iii) the CDR1 sequence of GLTFSSYR (SEQ ID NO: 20), the CDR2 sequence of IDWNGRGTYY (SEQ ID NO: 50) or IDWNGRGTYYR (SEQ ID NO: 270), and the CDR3 sequence of CAATTVLTDPRVLNEYAT (SEQ ID NO: 83) or AATTVLTDPRVLNEYAT (SEQ ID NO: 284); iv) the CDR1 sequence of GLTFSSYRMG (SEQ ID NO: 154), the CDR2 sequence of AIDWNGRGTYYRYYADSVKG (SEQ ID NO: 184), and the CDR3 sequence of TTVLTDPRVLNEYAT (SEQ ID NO: 215); v) the CDR1 sequence of SSYRMG (SEQ ID NO: 164), the CDR2 sequence of FVAAIDWNGRGTYYRY (SEQ ID NO: 194), and the CDR3 sequence of AATTVLTDPRVLNEYA (SEQ ID NO: 226); or vi) the CDR1 sequence of GLTFSSYRMG (SEQ ID NO: 174), the CDR2 sequence of AIDWNGRGTYYRY (SEQ ID NO: 204), and the CDR3 sequence of TTVLTDPRVLNEYAT (SEQ ID NO: 237).

In some embodiments, the VHH domain comprises a CDR1 sequence, a CDR2 sequence, and a CDR3 sequence present in VHH3. Accordingly, in some embodiments, the VHH domain comprises: i) the CDR1 sequence of INVMG (SEQ ID NO: 2), the CDR2 sequence of RINGGGITHYAESVKG (SEQ ID NO: 31), and the CDR3 sequence of DVFGSSGYVETY (SEQ ID NO: 62); ii) the CDR1 sequence of GSIFSIN (SEQ ID NO: 11), the CDR2 sequence of NGGGI (SEQ ID NO: 41) or GGG (SEQ ID NO: 261), and the CDR3 sequence of DVFGSSGYVETY (SEQ ID NO: 73) or VFGSSGYVET (SEQ ID NO: 274); iii) the CDR1 sequence of GSIFSINV (SEQ ID NO: 21), the CDR2 sequence of INGGGIT (SEQ ID NO: 51), and the CDR3 sequence of KADVFGSSGYVETY (SEQ ID NO: 84); iv) the CDR1 sequence of GSIFSINVMG (SEQ ID NO: 155), the CDR2 sequence of RINGGGITHYAESVKG (SEQ ID NO: 185), and the CDR3 sequence of DVFGSSGYVETY (SEQ ID NO: 216); v) the CDR1 sequence of SINVMG (SEQ ID NO: 165), the CDR2 sequence of LVARINGGGITH (SEQ ID NO: 195), and the CDR3 sequence of KADVFGSSGYVET (SEQ ID NO: 227); or vi) the CDR1 sequence of GSIFSINVMG (SEQ ID NO: 175), the CDR2 sequence of RINGGGITH (SEQ ID NO: 205), and the CDR3 sequence of DVFGSSGYVETY (SEQ ID NO: 238).

In some embodiments, the VHH domain comprises a CDR1 sequence, a CDR2 sequence, and a CDR3 sequence present in VHH4. Accordingly, in some embodiments, the VHH domain comprises: i) the CDR1 sequence of SNAMG (SEQ ID NO: 3), the CDR2 sequence of FIDRIATTTIATSVKG (SEQ ID NO: 32), and the CDR3 sequence of PLTAR (SEQ ID NO: 63); ii) the CDR1 sequence of GTSVSSN (SEQ ID NO: 12), the CDR2 sequence of DRIAT (SEQ ID NO: 42) or RIA (SEQ ID NO: 262), and the CDR3 sequence of PLTAR (SEQ ID NO: 74) or LTA (SEQ ID NO: 275); iii) the CDR1 sequence of GTSVSSNA (SEQ ID NO: 22), the CDR2 sequence of IDRIATT (SEQ ID NO: 52), and the CDR3 sequence of NHPLTAR (SEQ ID NO: 85); iv) the CDR1 sequence of GTSVSSNAMG (SEQ ID NO: 156), the CDR2 sequence of FIDRIATTTIATSVKG (SEQ ID NO: 186), and the CDR3 sequence of PLTAR (SEQ ID NO: 217); v) the CDR1 sequence of SSNAMG (SEQ ID NO: 166), the CDR2 sequence of WVGFIDRIATTT (SEQ ID NO: 196), and the CDR3 sequence of NHPLTA (SEQ ID NO: 228); or vi) the CDR1 sequence of GTSVSSNAMG (SEQ ID NO: 176), the CDR2 sequence of FIDRIATTT (SEQ ID NO: 206), and the CDR3 sequence of PLTAR (SEQ ID NO: 239).

In some embodiments, the VHH domain comprises a CDR1 sequence, a CDR2 sequence, and a CDR3 sequence present in VHH5. Accordingly, in some embodiments, the VHH domain comprises: i) the CDR1 sequence of SYAMG (SEQ ID NO: 4), the CDR2 sequence of AITWNGGTTYYADSVKG (SEQ ID NO: 33), and the CDR3 sequence of DPFNQGY (SEQ ID NO: 64); ii) the CDR1 sequence of GRTFSSY (SEQ ID NO: 13), the CDR2 sequence of TWNGGT (SEQ ID NO: 43) or WNGG (SEQ ID NO: 263), and the CDR3 sequence of DPFNQGY (SEQ ID NO: 75) or PFNQG (SEQ ID NO: 276); iii) the CDR1 sequence of GRTFSSYA (SEQ ID NO: 23), the CDR2 sequence of ITWNGGTT (SEQ ID NO: 53), and the CDR3 sequence of AADPFNQGY (SEQ ID NO: 86); iv) the CDR1 sequence of GRTFSSYAMG (SEQ ID NO: 157), the CDR2 sequence of AITWNGGTTYYADSVKG (SEQ ID NO: 187), and the CDR3 sequence of DPFNQGY (SEQ ID NO: 218); v) the CDR1 sequence of SSYAMG (SEQ ID NO: 167), the CDR2 sequence of FVAAITWNGGTTY (SEQ ID NO: 197), and the CDR3 sequence of AADPFNQG (SEQ ID NO: 229); or vi) the CDR1 sequence of GRTFSSYAMG (SEQ ID NO: 177), the CDR2 sequence of AITWNGGTTY (SEQ ID NO: 207), and the CDR3 sequence of DPFNQGY (SEQ ID NO: 240).

In some embodiments, the VHH domain comprises a CDR1 sequence, a CDR2 sequence, and a CDR3 sequence present in VHH6. Accordingly, in some embodiments, the VHH domain comprises: i) the CDR1 sequence of SDAMG (SEQ ID NO: 5), the CDR2 sequence of FISGGGTTTYADSVKG (SEQ ID NO: 34), and the CDR3 sequence of PLTSR (SEQ ID NO: 65); ii) the CDR1 sequence of GSSVSSD (SEQ ID NO: 14), the CDR2 sequence of SGGGT (SEQ ID NO: 44) or GGG (SEQ ID NO: 264), and the CDR3 sequence of PLTSR (SEQ ID NO: 76) or LTS (SEQ ID NO: 277); iii) the CDR1 sequence of GSSVSSDA (SEQ ID NO: 24), the CDR2 sequence of ISGGGTT (SEQ ID NO: 54), and the CDR3 sequence of NHPLTSR (SEQ ID NO: 87); iv) the CDR1 sequence of GSSVSSDAMG (SEQ ID NO: 158), the CDR2 sequence of FISGGGTTTYADSVKG (SEQ ID NO: 188), and the CDR3 sequence of PLTSR (SEQ ID NO: 219); v) the CDR1 sequence of SSDAMG (SEQ ID NO: 168), the CDR2 sequence of WVAFISGGGTTT (SEQ ID NO: 198), and the CDR3 sequence of NHPLTS (SEQ ID NO: 230); or vi) the CDR1 sequence of GSSVSSDAMG (SEQ ID NO: 178), the CDR2 sequence of FISGGGTTT (SEQ ID NO: 208), and the CDR3 sequence of PLTSR (SEQ ID NO: 241).

In some embodiments, the VHH domain comprises a CDR1 sequence, a CDR2 sequence, and a CDR3 sequence present in VHH7. Accordingly, in some embodiments, the VHH domain comprises: i) the CDR1 sequence of INVMG (SEQ ID NO: 6), the CDR2 sequence of RITGGGSTHYAESVKG (SEQ ID NO: 35), and the CDR3 sequence of MVNPIITAWGTIGVREIPDYDY (SEQ ID NO: 66); ii) the CDR1 sequence of RSIGSIN (SEQ ID NO: 15), the CDR2 sequence of TGGGS (SEQ ID NO: 45) or GGG (SEQ ID NO: 265), and the CDR3 sequence of MVNPIITAWGTIGVREIPDYDY (SEQ ID NO: 77) or VNPIITAWGTIGVREIPDYD (SEQ ID NO: 278); iii) the CDR1 sequence of RSIGSINV (SEQ ID NO: 25), the CDR2 sequence of ITGGGST (SEQ ID NO: 55), and the CDR3 sequence of ASMVNPIITAWGTIGVREIPDYDY (SEQ ID NO: 88); iv) the CDR1 sequence of RSIGSINVMG (SEQ ID NO: 159), the CDR2 sequence of RITGGGSTHYAESVKG (SEQ ID NO: 189), and the CDR3 sequence of MVNPIITAWGTIGVREIPDYDY (SEQ ID NO: 220); v) the CDR1 sequence of SINVMG (SEQ ID NO: 169), the CDR2 sequence of LVARITGGGSTH (SEQ ID NO: 199), and the CDR3 sequence of ASMVNPIITAWGTIGVREIPDYD (SEQ ID NO: 231); or vi) the CDR1 sequence of RSIGSINVMG (SEQ ID NO: 179), the CDR2 sequence of RITGGGSTH (SEQ ID NO: 209), and the CDR3 sequence of MVNPIITAWGTIGVREIPDYDY (SEQ ID NO: 242).

In some embodiments, the VHH domain comprises a CDR1 sequence, a CDR2 sequence, and a CDR3 sequence present in VHH9. Accordingly, in some embodiments, the VHH domain comprises: i) the CDR1 sequence of TYRMG (SEQ ID NO: 7), the CDR2 sequence of AISWSGGSTTYADPVKG (SEQ ID NO: 36), and the CDR3 sequence of DQRGY (SEQ ID NO: 67) or QRGY (SEQ ID NO: 271); ii) the CDR1 sequence of GRTFSTY (SEQ ID NO: 16), the CDR2 sequence of SWSGGS (SEQ ID NO: 46) or WSGG (SEQ ID NO: 266), and the CDR3 sequence of DQRGY (SEQ ID NO: 78) or RG (SEQ ID NO: 279); iii) the CDR1 sequence of GRTFSTYR (SEQ ID NO: 26), the CDR2 sequence of ISWSGGST (SEQ ID NO: 56), and the CDR3 sequence of NDQRGY (SEQ ID NO: 89); iv) the CDR1 sequence of GRTFSTYRMG (SEQ ID NO: 160), the CDR2 sequence of AISWSGGSTTYADPVKG (SEQ ID NO: 190), and the CDR3 sequence of QRGY (SEQ ID NO: 221); v) the CDR1 sequence of STYRMG (SEQ ID NO: 170), the CDR2 sequence of FVAAISWSGGSTT (SEQ ID NO: 200), and the CDR3 sequence of NDQRG (SEQ ID NO: 232); or vi) the CDR1 sequence of GRTFSTYRMG (SEQ ID NO: 180), the CDR2 sequence of AISWSGGSTT (SEQ ID NO: 210), and the CDR3 sequence of QRGY (SEQ ID NO: 243).

In some embodiments, the VHH domain comprises a CDR1 sequence, a CDR2 sequence, and a CDR3 sequence present in VHH10. Accordingly, in some embodiments, the VHH domain comprises: i) the CDR1 sequence of RYAMG (SEQ ID NO: 8), the CDR2 sequence of AISWSGSSAGYGDSVKG (SEQ ID NO: 37), and the CDR3 sequence of DPFNQGY (SEQ ID NO: 68); ii) the CDR1 sequence of GFTFTRY (SEQ ID NO: 17), the CDR2 sequence of SWSGSS (SEQ ID NO: 47) or WSGS (SEQ ID NO: 267), and the CDR3 sequence of DPFNQGY (SEQ ID NO: 79) or PFNQG (SEQ ID NO: 280); iii) the CDR1 sequence of GFTFTRYA (SEQ ID NO: 27), the CDR2 sequence of ISWSGSSA (SEQ ID NO: 57), and the CDR3 sequence of AADPFNQGY (SEQ ID NO: 90); iv) the CDR1 sequence of GFTFTRYAMG (SEQ ID NO: 161), the CDR2 sequence of AISWSGSSAGYGDSVKG (SEQ ID NO: 191), and the CDR3 sequence of DPFNQGY (SEQ ID NO: 222); v) the CDR1 sequence of TRYAMG (SEQ ID NO: 171), the CDR2 sequence of FVAAISWSGSSAG (SEQ ID NO: 201), and the CDR3 sequence of AADPFNQG (SEQ ID NO: 233); or vi) the CDR1 sequence of GFTFTRYAMG (SEQ ID NO: 181), the CDR2 sequence of AISWSGSSAG (SEQ ID NO: 211), and the CDR3 sequence of DPFNQGY (SEQ ID NO: 244).

In some embodiments, the VHH domain comprises a CDR1 sequence, a CDR2 sequence, and a CDR3 sequence present in VHH11. Accordingly, in some embodiments, the VHH domain comprises: i) the CDR1 sequence of FTTYRMG (SEQ ID NO: 258) or TYRMG (SEQ ID NO: 259), the CDR2 sequence of AIRWSGGRTLYADSVKG (SEQ ID NO: 38), and the CDR3 sequence of DLAEYSGTYSSPADSPAGYDY (SEQ ID NO: 69); ii) the CDR1 sequence of GRTFTTY (SEQ ID NO: 18), the CDR2 sequence of RWSGGR (SEQ ID NO: 48) or WSGG (SEQ ID NO: 268), and the CDR3 sequence of DLAEYSGTYSSPADSPAGYDY (SEQ ID NO: 80) or LAEYSGTYSSPADSPAGYD (SEQ ID NO: 281); iii) the CDR1 sequence of GRTFTTYR (SEQ ID NO: 28), the CDR2 sequence of IRWSGGRT (SEQ ID NO: 58), and the CDR3 sequence of AADLAEYSGTYSSPADSPAGYDY (SEQ ID NO: 91); iv) the CDR1 sequence of GRTFTTYRMG (SEQ ID NO: 162), the CDR2 sequence of AIRWSGGRTLYADSVKG (SEQ ID NO: 192), and the CDR3 sequence of DLAEYSGTYSSPADSPAGYDY (SEQ ID NO: 223); v) the CDR1 sequence of TTYRMG (SEQ ID NO: 172), the CDR2 sequence of FVAAIRWSGGRTL (SEQ ID NO: 202), and the CDR3 sequence of AADLAEYSGTYSSPADSPAGYD (SEQ ID NO: 234); or vi) the CDR1 sequence of GRTFTTYRMG (SEQ ID NO: 182), the CDR2 sequence of AIRWSGGRTL (SEQ ID NO: 212), and the CDR3 sequence of DLAEYSGTYSSPADSPAGYDY (SEQ ID NO: 245).

In some embodiments, the VHH domain comprises a CDR1 sequence, a CDR2 sequence, and a CDR3 sequence present in VHH12. Accordingly, in some embodiments, the VHH domain comprises: i) the CDR1 sequence of FNTYAMG (SEQ ID NO: 9), the CDR2 sequence of SITWNGGSTSYADSVKG (SEQ ID NO: 39), and the CDR3 sequence of ARYYVSGTYFPANY (SEQ ID NO: 70); ii) the CDR1 sequence of GRTLSFNTY (SEQ ID NO: 19), the CDR2 sequence of TWNGGS (SEQ ID NO: 49) or WNGG (SEQ ID NO: 269), and the CDR3 sequence of ARYYVSGTYFPANY (SEQ ID NO: 81) or RYYVSGTYFPAN (SEQ ID NO: 282); iii) the CDR1 sequence of GRTLSFNTYA (SEQ ID NO: 29), the CDR2 sequence of ITWNGGST (SEQ ID NO: 59), and the CDR3 sequence of AAARYYVSGTYFPANY (SEQ ID NO: 92); iv) the CDR1 sequence of GRTLSFNTYAMG (SEQ ID NO: 163), the CDR2 sequence of SITWNGGSTSYADSVKG (SEQ ID NO: 193), and the CDR3 sequence of ARYYVSGTYFPANY (SEQ ID NO: 224); v) the CDR1 sequence of SFNTYAMG (SEQ ID NO: 173), the CDR2 sequence of FVASITWNGGSTS (SEQ ID NO: 203), and the CDR3 sequence of AAARYYVSGTYFPAN (SEQ ID NO: 235); or vi) the CDR1 sequence of GRTLSFNTYAMG (SEQ ID NO: 183), the CDR2 sequence of SITWNGGSTS (SEQ ID NO: 213), and the CDR3 sequence of ARYYVSGTYFPANY (SEQ ID NO: 246).

In some embodiments, the VHH domain comprises a framework derived from the framework of any of the VHH domains comprising the sequences of

(SEQ ID NO: 93)
QVQLVESGGGLVQAGGSLKLACAAPGLTFSSYRMGWFRQAPGQERE
FVAAIDWNGRGTYYRYYADSVKGRSTISRDNAKNTMYLQMNSLKPE
DTAVYYCAAGSIDLNWYGGMDYWGQGTQVTVSS,
(SEQ ID NO: 94)
EVQVVESGGGLVQAGGSLKLACAAPGLTFSSYRMGWFRQAPGQERE
FVAAIDWNGRGTYYRYYADSVKGRSTISRDNAKNTVYLQMNSLKPE
DTAVYYCAATTVLTDPRVLNEYATWGQGTQVTVSS,
(SEQ ID NO: 95)
QLQLVESGGGLVQPGGSLRLSCAASGSIFSINVMGWYRQAPGKQRE
LVARINGGGITHYAESVKGRFTISRDNAKNTVYLQMNSLKPEDTAA
YYCKADVFGSSGYVETYWGQGTQVTVSS,
(SEQ ID NO: 96)
EVQVVESGGGLVQAGGSLRLSCAVSGTSVSSNAMGWYRQAPGKQRE
WVGFIDRIATTTIATSVKGRFAITRDNAKNTVYLQMSGLKPEDTAV
YYCNHPLTARWGQGTQVTVSS,
(SEQ ID NO: 97)
QVQLVESGGGLVQAGGSLRLSCAASGRTFSSYAMGWFRQAPGKERE
FVAAITWNGGTTYYADSVKGRFTISRDNAKNTVYLQMNSLKPEDTA
VYYCAADPFNQGYWGQGTQVTVSS,
(SEQ ID NO: 98)
EVQLVESGGGLVQAGGSLRLSCAVSGSSVSSDAMGWYRQAPGNQRA
WVAFISGGGTTTYADSVKGRFTISRDNTKNTVYLHMNSLKPEDTAV
YYCNHPLTSRWGQGTQVTVSS,
(SEQ ID NO: 99)
EVQVVESGGGLVQAGGSLRLACVASRSIGSINVMGWYRQAPGKQRD
LVARITGGGSTHYAESVKGRFTISRDNAKNTVYLQMNSLEPEDTAV
YYCASMVNPIITAWGTIGVREIPDYDYWGQGTQVTVSS,
(SEQ ID NO: 100)
QVQLVESGGGLVQAGGSLRLSCAVSGRTFSTYRMGWFRQAPGKERS
FVAAISWSGGSTTYADPVKGRFTISRDNAKNTVYLRMNSLKPEDTA
VYYCNDQRGYWGQGTLVTVSS,
(SEQ ID NO: 101)
EVQVVESGGGLVQAGGSLRLSCAASGFTFTRYAMGWFRQAPGKERS
FVAAISWSGSSAGYGDSVKGRFTISRDNAKNTLYLQMNSLKPEDTA
VYYCAADPFNQGYWGQGTQVTVSS,
(SEQ ID NO: 102)
EVQVVESGGGLVQAGGSLRLSCAASGRTFTTYRMGWFRQAPGKERE
FVAAIRWSGGRTLYADSVKGRFTISRDNAKNTAYLQMNNLRPEDTA
VYYCAADLAEYSGTYSSPADSPAGYDYWGQGTQVTVSS,
or
(SEQ ID NO: 103)
QVQLVETGGGLVQAGDSLRLSCAASGRTLSFNTYAMGWFRQAPGKE
REFVASITWNGGSTSYADSVKGRFTITRDNAKNTATLRMNSLQPDD
TAVYYCAAARYYVSGTYFPANYWGQGTQVTVSS.

In some embodiments, the VHH domain comprises a framework comprising sequence having at least 75%, 80%, 85%, 90%, 95%, 96%, 97%, 98%, 99%, or 100% sequence identity with the sequence of

(SEQ ID NO: 93)
QVQLVESGGGLVQAGGSLKLACAAPGLTFSSYRMGWFRQAPGQERE
FVAAIDWNGRGTYYRYYADSVKGRSTISRDNAKNTMYLQMNSLKPE
DTAVYYCAAGSIDLNWYGGMDYWGQGTQVTVSS,
(SEQ ID NO: 94)
EVQVVESGGGLVQAGGSLKLACAAPGLTFSSYRMGWFRQAPGQERE
FVAAIDWNGRGTYYRYYADSVKGRSTISRDNAKNTVYLQMNSLKPE
DTAVYYCAATTVLTDPRVLNEYATWGQGTQVTVSS,
(SEQ ID NO: 95)
QLQLVESGGGLVQPGGSLRLSCAASGSIFSINVMGWYRQAPGKQRE
LVARINGGGITHYAESVKGRFTISRDNAKNTVYLQMNSLKPEDTAA
YYCKADVFGSSGYVETYWGQGTQVTVSS,
(SEQ ID NO: 96)
EVQVVESGGGLVQAGGSLRLSCAVSGTSVSSNAMGWYRQAPGKQRE
WVGFIDRIATTTIATSVKGRFAITRDNAKNTVYLQMSGLKPEDTAV
YYCNHPLTARWGQGTQVTVSS,
(SEQ ID NO: 97)
QVQLVESGGGLVQAGGSLRLSCAASGRTFSSYAMGWFRQAPGKERE
FVAAITWNGGTTYYADSVKGRFTISRDNAKNTVYLQMNSLKPEDTA
VYYCAADPFNQGYWGQGTQVTVSS,
(SEQ ID NO: 98)
EVQLVESGGGLVQAGGSLRLSCAVSGSSVSSDAMGWYRQAPGNQRA
WVAFISGGGTTTYADSVKGRFTISRDNTKNTVYLHMNSLKPEDTAV
YYCNHPLTSRWGQGTQVTVSS,
(SEQ ID NO: 99)
EVQVVESGGGLVQAGGSLRLACVASRSIGSINVMGWYRQAPGKQRD
LVARITGGGSTHYAESVKGRFTISRDNAKNTVYLQMNSLEPEDTAV
YYCASMVNPIITAWGTIGVREIPDYDYWGQGTQVTVSS,
(SEQ ID NO: 100)
QVQLVESGGGLVQAGGSLRLSCAVSGRTFSTYRMGWFRQAPGKERS
FVAAISWSGGSTTYADPVKGRFTISRDNAKNTVYLRMNSLKPEDTA
VYYCNDQRGYWGQGTLVTVSS,
(SEQ ID NO: 101)
EVQVVESGGGLVQAGGSLRLSCAASGFTFTRYAMGWFRQAPGKERS
FVAAISWSGSSAGYGDSVKGRFTISRDNAKNTLYLQMNSLKPEDTA
VYYCAADPFNQGYWGQGTQVTVSS,
(SEQ ID NO: 102)
EVQVVESGGGLVQAGGSLRLSCAASGRTFTTYRMGWFRQAPGKERE
FVAAIRWSGGRTLYADSVKGRFTISRDNAKNTAYLQMNNLRPEDTA
VYYCAADLAEYSGTYSSPADSPAGYDYWGQGTQVTVSS,
or
(SEQ ID NO: 103)
QVQLVETGGGLVQAGDSLRLSCAASGRTLSFNTYAMGWFRQAPGKE
REFVASITWNGGSTSYADSVKGRFTITRDNAKNTATLRMNSLQPDD
TAVYYCAAARYYVSGTYFPANYWGQGTQVTVSS.

In some embodiments, the VHH domain is comprised of a sequence having at least 75%, 80%, 85%, 90%, 95%, 96%, 97%, 98%, 99%, or 100% sequence identity with the sequence of

(SEQ ID NO: 93)
QVQLVESGGGLVQAGGSLKLACAAPGLTFSSYRMGWFRQAPGQERE
FVAAIDWNGRGTYYRYYADSVKGRSTISRDNAKNTMYLQMNSLKPE
DTAVYYCAAGSIDLNWYGGMDYWGQGTQVTVSS,
(SEQ ID NO: 94)
EVQVVESGGGLVQAGGSLKLACAAPGLTFSSYRMGWFRQAPGQERE
FVAAIDWNGRGTYYRYYADSVKGRSTISRDNAKNTVYLQMNSLKPE
DTAVYYCAATTVLTDPRVLNEYATWGQGTQVTVSS,
(SEQ ID NO: 95)
QLQLVESGGGLVQPGGSLRLSCAASGSIFSINVMGWYRQAPGKQRE
LVARINGGGITHYAESVKGRFTISRDNAKNTVYLQMNSLKPEDTAA
YYCKADVFGSSGYVETYWGQGTQVTVSS,
(SEQ ID NO: 96)
EVQVVESGGGLVQAGGSLRLSCAVSGTSVSSNAMGWYRQAPGKQRE
WVGFIDRIATTTIATSVKGRFAITRDNAKNTVYLQMSGLKPEDTAV
YYCNHPLTARWGQGTQVTVSS,
(SEQ ID NO: 97)
QVQLVESGGGLVQAGGSLRLSCAASGRTFSSYAMGWFRQAPGKERE
FVAAITWNGGTTYYADSVKGRFTISRDNAKNTVYLQMNSLKPEDTA
VYYCAADPFNQGYWGQGTQVTVSS,
(SEQ ID NO: 98)
EVQLVESGGGLVQAGGSLRLSCAVSGSSVSSDAMGWYRQAPGNQRA
WVAFISGGGTTTYADSVKGRFTISRDNTKNTVYLHMNSLKPEDTAV
YYCNHPLTSRWGQGTQVTVSS,
(SEQ ID NO: 99)
EVQVVESGGGLVQAGGSLRLACVASRSIGSINVMGWYRQAPGKQRD
LVARITGGGSTHYAESVKGRFTISRDNAKNTVYLQMNSLEPEDTAV
YYCASMVNPIITAWGTIGVREIPDYDYWGQGTQVTVSS,
(SEQ ID NO: 100)
QVQLVESGGGLVQAGGSLRLSCAVSGRTFSTYRMGWFRQAPGKERS
FVAAISWSGGSTTYADPVKGRFTISRDNAKNTVYLRMNSLKPEDTA
VYYCNDQRGYWGQGTLVTVSS,
(SEQ ID NO: 101)
EVQVVESGGGLVQAGGSLRLSCAASGFTFTRYAMGWFRQAPGKERS
FVAAISWSGSSAGYGDSVKGRFTISRDNAKNTLYLQMNSLKPEDTA
VYYCAADPFNQGYWGQGTQVTVSS,
(SEQ ID NO: 102)
EVQVVESGGGLVQAGGSLRLSCAASGRTFTTYRMGWFRQAPGKERE
FVAAIRWSGGRTLYADSVKGRFTISRDNAKNTAYLQMNNLRPEDTA
VYYCAADLAEYSGTYSSPADSPAGYDYWGQGTQVTVSS,
or
(SEQ ID NO: 103)
QVQLVETGGGLVQAGDSLRLSCAASGRTLSFNTYAMGWFRQAPGKE
REFVASITWNGGSTSYADSVKGRFTITRDNAKNTATLRMNSLQPDD
TAVYYCAAARYYVSGTYFPANYWGQGTQVTVSS.

In another aspect is provided an isolated nucleic acid molecule encoding any of the above VHH domains.

In another aspect is provided an isolated nucleic acid molecule encoding the VHH domain having a sequence with at least 85%, 90%, 95%, 96%, 97%, 98%, 99% or 100% sequence identity to the sequence of QVQLVESGGGLVQAGGSLKLACAAPGLTFSSYRMGWFRQAPGQEREFVAAIDWNGRG TYYRYYADSVKGRSTISRDNAKNTMYLQMNSLKPEDTAVYYCAAGSIDLNWYGGMD YWGQGTQVTVSS (SEQ ID NO: 93), EVQVVESGGGLVQAGGSLKLACAAPGLTFSSYRMGWFRQAPGQEREFVAAIDWNGRG TYYRYYADSVKGRSTISRDNAKNTVYLQMNSLKPEDTAVYYCAATTVLTDPRVLNEYA TWGQGTQVTVSS (SEQ ID NO: 94), QLQLVESGGGLVQPGGSLRL SCAASGSIFSINVMGWYRQAPGKQRELVARINGGGITHY AESVKGRFTISRDNAKNTVYLQMNSLKPEDTAAYYCKADVFGSSGYVETYWGQGTQV TVSS (SEQ ID NO: 95), EVQVVESGGGLVQAGGSLRLSCAVSGTSVSSNAMGWYRQAPGKQREWVGFIDRIATTT IATSVKGRFAITRDNAKNTVYLQMSGLKPEDTAVYYCNHPLTARWGQGTQVTVSS (SEQ ID NO: 96), QVQLVESGGGLVQAGGSLRL SCAASGRTFSSYAMGWFRQAPGKEREFVAAITWNGGTT YYADSVKGRFTISRDNAKNTVYLQMNSLKPEDTAVYYCAADPFNQGYWGQGTQVTVS S (SEQ ID NO: 97), EVQLVESGGGLVQAGGSLRLSCAVSGSSVSSDAMGWYRQAPGNQRAWVAFISGGGTT TYADSVKGRFTISRDNTKNTVYLHMNSLKPEDTAVYYCNHPLTSRWGQGTQVTVSS (SEQ ID NO: 98), EVQVVESGGGLVQAGGSLRLACVASRSIGSINVMGWYRQAPGKQRDLVARITGGGSTH YAESVKGRFTISRDNAKNTVYLQMNSLEPEDTAVYYCASMVNPIITAWGTIGVREIPDY DYWGQGTQVTVSS (SEQ ID NO: 99), QVQLVESGGGLVQAGGSLRLSCAVSGRTFSTYRMGWFRQAPGKERSFVAAISWSGGST TYADPVKGRFTISRDNAKNTVYLRMNSLKPEDTAVYYCNDQRGYWGQGTLVTVSS (SEQ ID NO: 100), EVQVVESGGGLVQAGGSLRLSCAASGFTFTRYAMGWFRQAPGKERSFVAAISWSGSSA GYGDSVKGRF TISRDNAKNTLYLQMNSLKPEDTAVYYCAADPFNQGYWGQGTQVTVS S (SEQ ID NO: 101), EVQVVESGGGLVQAGGSLRL SCAASGRTFTTYRMGWFRQAPGKEREFVAAIRWSGGRT LYADSVKGRF TISRDNAKNTAYLQMNNLRPED TAVYYCAADLAEYSGTYSSPADSPAG YDYWGQGTQVTVSS (SEQ ID NO: 102), or QVQLVETGGGLVQAGDSLRLSCAASGRTLSFNTYAMGWFRQAPGKEREFVASITWNG GSTSYADSVKGRFTITRDNAKNTATLRMNSLQPDDTAVYYCAAARYYVSGTYFPANY WGQGTQVTVSS (SEQ ID NO: 103), optionally wherein the nucleic acid molecule comprises a sequence with at least 85%, 90%, 95%, 96%, 97%, 98%, 99% or 100% sequence identity to the polynucleotide sequence of any one of SEQ ID NOS: 133-142.

In another aspect is provided an vector comprising any of the above nucleic acid molecules. In another aspect is provided a cell expressing any of the above nucleic acid molecules.

In another aspect is provided a pharmaceutical composition comprising any of the above VHH domains and a pharmaceutically acceptable excipient. In another aspect is provided a pharmaceutical composition comprising a means for delivering a molecule in systemic circulation in a subject, and a pharmaceutically acceptable carrier. In another aspect is provided a pharmaceutical composition comprising a means for delivering a molecule into Lamina propria of a subject, and a pharmaceutically acceptable carrier. In another aspect is provided a pharmaceutical composition comprising a means for delivering a molecule to a mucosal lumen of a subject, and a pharmaceutically acceptable carrier. In another aspect is provided a pharmaceutical composition comprising a means for delivering a molecule to an organ of a subject, and a pharmaceutically acceptable carrier. In another aspect is provided a pharmaceutical composition comprising a means for delivering a molecule to a pIgR-expressing cell, and a pharmaceutically acceptable carrier. In various embodiments of these aspects, the molecule is an antibody or fragment thereof, a peptide, a vaccine, a small molecule, a polynucleotide, a molecule comprising a radioactive isotope, a toxin, an enzyme, an anticoagulant, a hormone, a cytokine, an anti-inflammatory molecule, an RNAi, an mRNA, a self-replicating RNA, an antibiotic, or an antibody-antibiotic conjugate.

In one aspect is provided a therapeutic molecule comprising an agent and a VHH domain that binds to an extracellular domain of pIgR. In some embodiments, the VHH domain binds to an extracellular domain 1 of pIgR. In some embodiments, the VHH domain binds to an extracellular domain 2 of pIgR. In some embodiments, the VHH domain binds to an extracellular domain 1-2 of pIgR. In some embodiments, the VHH domain binds to an extracellular domain 3 of pIgR. In some embodiments, the VHH domain binds to an extracellular domain 2-3 of pIgR. In some embodiments, the VHH domain binds to an extracellular domain 4-5 of pIgR. In some embodiments, the VHH domain binds to an extracellular domain 5 of pIgR. In some embodiments, the pIgR is human pIgR. In some embodiments, the pIgR is mouse pIgR. In some embodiments, the VHH domain does not detectably bind to the amino acid sequence of EKAVADTRDQADGSRASVDSGSSEEQGGSSR (SEQ ID NO: 143), EREIQNVGDQAQENRASGDAGSADGQSRSSSSK (SEQ ID NO: 144), or

EREIQNVRDQAQENRASGDAGSADGQSRSSSSK (SEQ ID NO: 145). In some embodiments, the VHH domain competes with IgA binding to the pIgR. In some embodiments, the VHH domain promotes IgA binding to the pIgR. In some embodiments, the K_{D} of the binding of the VHH domain to pIgR is from about 4 to 525 nM. In some embodiments, the K_{D} of the binding of the VHH domain to pIgR is less than about 50 nM. In some embodiments, the K_{D} of the binding of the VHH domain to pIgR is from about 4 to about 34 nM. In some embodiments, the Tₘ of the VHH domain is from about 53 to about 77° C. In some embodiments, the Tₘ of the VHH domain is from 53.9 to 76.4° C.

In some embodiments, provided is a therapeutic molecule comprising an agent and a VHH domain that binds to an extracellular domain of pIgR, wherein the VHH domain comprises a CDR1 sequence of SYRMG (SEQ ID NO: 1), INVMG (SEQ ID NO: 2), SNAMG (SEQ ID NO: 3), SYAMG (SEQ ID NO: 4), SDAMG (SEQ ID NO: 5), INVMG (SEQ ID NO: 6), TYRMG (SEQ ID NO: 7), RYAMG (SEQ ID NO: 8), FTTYRMG (SEQ ID NO: 258), TYRMG (SEQ ID NO: 259), FNTYAMG (SEQ ID NO: 9), GLTFSSY (SEQ ID NO: 10), GSIFSIN (SEQ ID NO: 11), GTSVSSN (SEQ ID NO: 12), GRTFSSY (SEQ ID NO: 13), GSSVSSD (SEQ ID NO: 14), RSIGSIN (SEQ ID NO: 15), GRTFSTY (SEQ ID NO: 16), GFTFTRY (SEQ ID NO: 17), GRTFTTY (SEQ ID NO: 18), GRTLSFNTY (SEQ ID NO: 19), GLTFSSYR (SEQ ID NO: 20), GSIFSINV (SEQ ID NO: 21), GTSVSSNA (SEQ ID NO: 22), GRTFSSYA (SEQ ID NO: 23), GSSVSSDA (SEQ ID NO: 24), RSIGSINV (SEQ ID NO: 25), GRTFSTYR (SEQ ID NO: 26), GFTFTRYA (SEQ ID NO: 27), GRTFTTYR (SEQ ID NO: 28), GRTLSFNTYA (SEQ ID NO: 29), GLTFSSYRMG (SEQ ID NO: 154), GSIFSINVMG (SEQ ID NO: 155), GTSVSSNAMG (SEQ ID NO: 156), GRTFSSYAMG (SEQ ID NO: 157), GSSVSSDAMG (SEQ ID NO: 158), RSIGSINVMG (SEQ ID NO: 159), GRTFSTYRMG (SEQ ID NO: 160), GFTFTRYAMG (SEQ ID NO: 161), GRTFTTYRMG (SEQ ID NO: 162), GRTLSFNTYAMG (SEQ ID NO: 163), SSYRMG (SEQ ID NO: 164), SINVMG (SEQ ID NO: 165), SSNAMG (SEQ ID NO: 166), SSYAMG (SEQ ID NO: 167), SSDAMG (SEQ ID NO: 168), SINVMG (SEQ ID NO: 169), STYRMG (SEQ ID NO: 170), TRYAMG (SEQ ID NO: 171), TTYRMG (SEQ ID NO: 172), SFNTYAMG (SEQ ID NO: 173), GLTFSSYRMG (SEQ ID NO: 174), GSIFSINVMG (SEQ ID NO: 175), GTSVSSNAMG (SEQ ID NO: 176), GRTFSSYAMG (SEQ ID NO: 177), GSSVSSDAMG (SEQ ID NO: 178), RSIGSINVMG (SEQ ID NO: 179), GRTFSTYRMG (SEQ ID NO: 180), GFTFTRYAMG (SEQ ID NO: 181), GRTFTTYRMG (SEQ ID NO: 182), or GRTLSFNTYAMG (SEQ ID NO: 183).

In some embodiments, provided is a therapeutic molecule comprising an agent and a VHH domain that binds to an extracellular domain of pIgR, wherein the VHH domain comprises a CDR2 sequence of AIDWNGRGTYYRYYADSVKG (SEQ ID NO: 30), RINGGGITHYAESVKG (SEQ ID NO: 31), FIDRIATTTIATSVKG (SEQ ID NO: 32), AITWNGGTTYYADSVKG (SEQ ID NO: 33), FISGGGTTTYADSVKG (SEQ ID NO: 34), RITGGGSTHYAESVKG (SEQ ID NO: 35), AISWSGGSTTYADPVKG (SEQ ID NO: 36), AISWSGSSAGYGDSVKG (SEQ ID NO: 37), AIRWSGGRTLYADSVKG (SEQ ID NO: 38), SITWNGGSTSYADSVKG (SEQ ID NO: 39), DWNGRGTYY (SEQ ID NO: 40), WNGRGTY (SEQ ID NO: 260), NGGGI (SEQ ID NO: 41), GGG (SEQ ID NO: 261), DRIAT (SEQ ID NO: 42), RIA (SEQ ID NO: 262), TWNGGT (SEQ ID NO: 43), WNGG (SEQ ID NO: 263), SGGGT (SEQ ID NO: 44), GGG (SEQ ID NO: 264), TGGGS (SEQ ID NO: 45), GGG (SEQ ID NO: 265), SWSGGS (SEQ ID NO: 46), WSGG (SEQ ID NO: 266), SWSGSS (SEQ ID NO: 47), WSGS (SEQ ID NO: 267), RWSGGR (SEQ ID NO: 48), WSGG (SEQ ID NO: 268), TWNGGS (SEQ ID NO: 49), WNGG (SEQ ID NO: 269), IDWNGRGTYY (SEQ ID NO: 50), IDWNGRGTYYR (SEQ ID NO: 270), INGGGIT (SEQ ID NO: 51), IDRIATT (SEQ ID NO: 52), ITWNGGTT (SEQ ID NO: 53), ISGGGTT (SEQ ID NO: 54), ITGGGST (SEQ ID NO: 55), ISWSGGST (SEQ ID NO: 56), ISWSGSSA (SEQ ID NO: 57), IRWSGGRT (SEQ ID NO: 58), ITWNGGST (SEQ ID NO: 59), AIDWNGRGTYYRYYADSVKG (SEQ ID NO: 184), RINGGGITHYAESVKG (SEQ ID NO: 185), FIDRIATTTIATSVKG (SEQ ID NO: 186), AITWNGGTTYYADSVKG (SEQ ID NO: 187), FISGGGTTTYADSVKG (SEQ ID NO: 188), RITGGGSTHYAESVKG (SEQ ID NO: 189), AISWSGGSTTYADPVKG (SEQ ID NO: 190), AISWSGSSAGYGDSVKG (SEQ ID NO: 191), AIRWSGGRTLYADSVKG (SEQ ID NO: 192), SITWNGGSTSYADSVKG (SEQ ID NO: 193), FVAAIDWNGRGTYYRY (SEQ ID NO: 194), LVARINGGGITH (SEQ ID NO: 195), WVGFIDRIATTT (SEQ ID NO: 196), FVAAITWNGGTTY (SEQ ID NO: 197), WVAFISGGGTTT (SEQ ID NO: 198), LVARITGGGSTH (SEQ ID NO: 199), FVAAISWSGGSTT (SEQ ID NO: 200), FVAAISWSGSSAG (SEQ ID NO: 201), FVAAIRWSGGRTL (SEQ ID NO: 202), FVASITWNGGSTS (SEQ ID NO: 203), AIDWNGRGTYYRY (SEQ ID NO: 204), RINGGGITH (SEQ ID NO: 205), FIDRIATTT (SEQ ID NO: 206), AITWNGGTTY (SEQ ID NO: 207), FISGGGTTT (SEQ ID NO: 208), RITGGGSTH (SEQ ID NO: 209), AISWSGGSTT (SEQ ID NO: 210), AISWSGSSAG (SEQ ID NO: 211), AIRWSGGRTL (SEQ ID NO: 212), or SITWNGGSTS (SEQ ID NO: 213).

In some embodiments, provided is a therapeutic molecule comprising an agent and a VHH domain that binds to an extracellular domain of pIgR, wherein the VHH domain comprises a CDR3 sequence of GSIDLNWYGGMDY (SEQ ID NO: 60), TTVLTDPRVLNEYAT (SEQ ID NO: 61), DVFGSSGYVETY (SEQ ID NO: 62), PLTAR (SEQ ID NO: 63), DPFNQGY (SEQ ID NO: 64), PLTSR (SEQ ID NO: 65), MVNPIITAWGTIGVREIPDYDY (SEQ ID NO: 66), DQRGY (SEQ ID NO: 67), QRGY (SEQ ID NO: 271), DPFNQGY (SEQ ID NO: 68), DLAEYSGTYSSPADSPAGYDY (SEQ ID NO: 69), ARYYVSGTYFPANY (SEQ ID NO: 70), GSIDLNWYGGMDY (SEQ ID NO: 71), SIDLNWYGGMD (SEQ ID NO: 272), TTVLTDPRVLNEYAT (SEQ ID NO: 72), TVLTDPRVLNEYA (SEQ ID NO: 273), DVFGSSGYVETY (SEQ ID NO: 73), VFGSSGYVET (SEQ ID NO: 274), PLTAR (SEQ ID NO: 74), LTA (SEQ ID NO: 275), DPFNQGY (SEQ ID NO: 75), PFNQG (SEQ ID NO: 276), PLTSR (SEQ ID NO: 76), LTS (SEQ ID NO: 277), MVNPIITAWGTIGVREIPDYDY (SEQ ID NO: 77), VNPIITAWGTIGVREIPDYD (SEQ ID NO: 278), DQRGY (SEQ ID NO: 78), RG (SEQ ID NO: 279), DPFNQGY (SEQ ID NO: 79), PFNQG (SEQ ID NO: 280), DLAEYSGTYSSPADSPAGYDY (SEQ ID NO: 80), LAEYSGTYSSPADSPAGYD (SEQ ID NO: 281), ARYYVSGTYFPANY (SEQ ID NO: 81), RYYVSGTYFPAN (SEQ ID NO: 282), CAAGSIDLNWYGGMDY (SEQ ID NO: 82), AAGSIDLNWYGGMDY (SEQ ID NO: 283), CAATTVLTDPRVLNEYAT (SEQ ID NO: 83), AATTVLTDPRVLNEYAT (SEQ ID NO: 284), KADVFGSSGYVETY (SEQ ID NO: 84), NHPLTAR (SEQ ID NO: 85), AADPFNQGY (SEQ ID NO: 86), NHPLTSR (SEQ ID NO: 87), ASMVNPIITAWGTIGVREIPDYDY (SEQ ID NO: 88), NDQRGY (SEQ ID NO: 89), AADPFNQGY (SEQ ID NO: 90), AADLAEYSGTYSSPADSPAGYDY (SEQ ID NO: 91), AAARYYVSGTYFPANY (SEQ ID NO: 92), GSIDLNWYGGMDY (SEQ ID NO: 214), TTVLTDPRVLNEYAT (SEQ ID NO: 215), DVFGSSGYVETY (SEQ ID NO: 216), PLTAR (SEQ ID NO: 217), DPFNQGY (SEQ ID NO: 218), PLTSR (SEQ ID NO: 219), MVNPIITAWGTIGVREIPDYDY (SEQ ID NO: 220), QRGY (SEQ ID NO: 221), DPFNQGY (SEQ ID NO: 222), DLAEYSGTYSSPADSPAGYDY (SEQ ID NO: 223), ARYYVSGTYFPANY (SEQ ID NO: 224), AAGSIDLNWYGGMD (SEQ ID NO: 225), AATTVLTDPRVLNEYA (SEQ ID NO: 226), KADVFGSSGYVET (SEQ ID NO: 227), NHPLTA (SEQ ID NO: 228), AADPFNQG (SEQ ID NO: 229), NHPLTS (SEQ ID NO: 230), ASMVNPIITAWGTIGVREIPDYD (SEQ ID NO: 231), NDQRG (SEQ ID NO: 232), AADPFNQG (SEQ ID NO: 233), AADLAEYSGTYSSPADSPAGYD (SEQ ID NO: 234), AAARYYVSGTYFPAN (SEQ ID NO: 235), GSIDLNWYGGMDY (SEQ ID NO: 236), TTVLTDPRVLNEYAT (SEQ ID NO: 237), DVFGSSGYVETY (SEQ ID NO: 238), PLTAR (SEQ ID NO: 239), DPFNQGY (SEQ ID NO: 240), PLTSR (SEQ ID NO: 241), MVNPIITAWGTIGVREIPDYDY (SEQ ID NO: 242), QRGY (SEQ ID NO: 243), DPFNQGY (SEQ ID NO: 244), DLAEYSGTYSSPADSPAGYDY (SEQ ID NO: 245), or ARYYVSGTYFPANY (SEQ ID NO: 246).

In some embodiments, provided is a therapeutic molecule comprising an agent and a VHH domain that binds to an extracellular domain of pIgR, wherein the VHH domain comprises a CDR1 sequence, a CDR2 sequence, and a CDR3 sequence present in VHH1. Accordingly, in some embodiments, the VHH domain comprises: i) the CDR1 sequence of SYRMG (SEQ ID NO: 1), the CDR2 sequence of AIDWNGRGTYYRYYADSVKG (SEQ ID NO: 30), and the CDR3 sequence of GSIDLNWYGGMDY (SEQ ID NO: 60); ii) the CDR1 sequence of GLTFSSY (SEQ ID NO: 10), the CDR2 sequence of DWNGRGTYY (SEQ ID NO: 40) or WNGRGTY (SEQ ID NO: 260), and the CDR3 sequence of GSIDLNWYGGMDY (SEQ ID NO: 71) or SIDLNWYGGMD (SEQ ID NO: 272); iii) the CDR1 sequence of GLTFSSYR (SEQ ID NO: 20), the CDR2 sequence of IDWNGRGTYY (SEQ ID NO: 50) or IDWNGRGTYYR (SEQ ID NO: 270), and the CDR3 sequence of CAAGSIDLNWYGGMDY (SEQ ID NO: 82) or AAGSIDLNWYGGMDY (SEQ ID NO: 283); iv) the CDR1 sequence of GLTFSSYRMG (SEQ ID NO: 154), the CDR 2 sequence of AIDWNGRGTYYRYYADSVKG (SEQ ID NO: 184), and the CDR3 sequence of GSIDLNWYGGMDY (SEQ ID NO: 214); v) the CDR1 sequence of SSYRMG (SEQ ID NO: 164), the CDR2 sequence of FVAAIDWNGRGTYYRY (SEQ ID NO: 194), and the CDR3 sequence of AAGSIDLNWYGGMD (SEQ ID NO: 225); or vi) the CDR1 sequence of GLTFSSYRMG (SEQ ID NO: 174), the CDR2 sequence of AIDWNGRGTYYRY (SEQ ID NO: 204), and the CDR3 sequence of GSIDLNWYGGMDY (SEQ ID NO: 236).

In some embodiments, provided is a therapeutic molecule comprising an agent and a VHH domain that binds to an extracellular domain of pIgR, wherein the VHH domain comprises a CDR1 sequence, a CDR2 sequence, and a CDR3 sequence present in VHH2. Accordingly, in some embodiments, the VHH domain comprises: i) the CDR1 sequence of SYRMG (SEQ ID NO: 1), the CDR2 sequence of AIDWNGRGTYYRYYADSVKG (SEQ ID NO: 30), and the CDR3 sequence of TTVLTDPRVLNEYAT (SEQ ID NO: 61); ii) the CDR1 sequence of GLTFSSY (SEQ ID NO: 10), the CDR2 sequence of DWNGRGTYY (SEQ ID NO: 40) or WNGRGTY (SEQ ID NO: 260), and the CDR3 sequence of TTVLTDPRVLNEYAT (SEQ ID NO: 72) or TVLTDPRVLNEYA (SEQ ID NO: 273); iii) the CDR1 sequence of GLTFSSYR (SEQ ID NO: 20), the CDR2 sequence of IDWNGRGTYY (SEQ ID NO: 50) or IDWNGRGTYYR (SEQ ID NO: 270), and the CDR3 sequence of CAATTVLTDPRVLNEYAT (SEQ ID NO: 83) or AATTVLTDPRVLNEYAT (SEQ ID NO: 284); iv) the CDR1 sequence of GLTFSSYRMG (SEQ ID NO: 154), the CDR2 sequence of AIDWNGRGTYYRYYADSVKG (SEQ ID NO: 184), and the CDR3 sequence of TTVLTDPRVLNEYAT (SEQ ID NO: 215); v) the CDR1 sequence of SSYRMG (SEQ ID NO: 164), the CDR2 sequence of FVAAIDWNGRGTYYRY (SEQ ID NO: 194), and the CDR3 sequence of AATTVLTDPRVLNEYA (SEQ ID NO: 226); or vi) the CDR1 sequence of GLTFSSYRMG (SEQ ID NO: 174), the CDR2 sequence of AIDWNGRGTYYRY (SEQ ID NO: 204), and the CDR3 sequence of TTVLTDPRVLNEYAT (SEQ ID NO: 237).

In some embodiments, provided is a therapeutic molecule comprising an agent and a VHH domain that binds to an extracellular domain of pIgR, wherein the VHH domain comprises a CDR1 sequence, a CDR2 sequence, and a CDR3 sequence present in VHH3. Accordingly, in some embodiments, the VHH domain comprises: i) the CDR1 sequence of INVMG (SEQ ID NO: 2), the CDR2 sequence of RINGGGITHYAESVKG (SEQ ID NO: 31), and the CDR3 sequence of DVFGSSGYVETY (SEQ ID NO: 62); ii) the CDR1 sequence of GSIFSIN (SEQ ID NO: 11), the CDR2 sequence of NGGGI (SEQ ID NO: 41) or GGG (SEQ ID NO: 261), and the CDR3 sequence of DVFGSSGYVETY (SEQ ID NO: 73) or VFGSSGYVET (SEQ ID NO: 274); iii) the CDR1 sequence of GSIFSINV (SEQ ID NO: 21), the CDR2 sequence of INGGGIT (SEQ ID NO: 51), and the CDR3 sequence of KADVFGSSGYVETY (SEQ ID NO: 84); iv) the CDR1 sequence of GSIFSINVMG (SEQ ID NO: 155), the CDR2 sequence of RINGGGITHYAESVKG (SEQ ID NO: 185), and the CDR3 sequence of DVFGSSGYVETY (SEQ ID NO: 216); v) the CDR1 sequence of SINVMG (SEQ ID NO: 165), the CDR2 sequence of LVARINGGGITH (SEQ ID NO: 195), and the CDR3 sequence of KADVFGSSGYVET (SEQ ID NO: 227); or vi) the CDR1 sequence of GSIFSINVMG (SEQ ID NO: 175), the CDR2 sequence of RINGGGITH (SEQ ID NO: 205), and the CDR3 sequence of DVFGSSGYVETY (SEQ ID NO: 238).

In some embodiments, provided is a therapeutic molecule comprising an agent and a VHH domain that binds to an extracellular domain of pIgR, wherein the VHH domain comprises a CDR1 sequence, a CDR2 sequence, and a CDR3 sequence present in VHH4. Accordingly, in some embodiments, the VHH domain comprises: i) the CDR1 sequence of SNAMG (SEQ ID NO: 3), the CDR2 sequence of FIDRIATTTIATSVKG (SEQ ID NO: 32), and the CDR3 sequence of PLTAR (SEQ ID NO: 63); ii) the CDR1 sequence of GTSVSSN (SEQ ID NO: 12), the CDR2 sequence of DRIAT (SEQ ID NO: 42) or RIA (SEQ ID NO: 262), and the CDR3 sequence of PLTAR (SEQ ID NO: 74) or LTA (SEQ ID NO: 275); iii) the CDR1 sequence of GTSVSSNA (SEQ ID NO: 22), the CDR2 sequence of IDRIATT (SEQ ID NO: 52), and the CDR3 sequence of NHPLTAR (SEQ ID NO: 85); iv) the CDR1 sequence of GTSVSSNAMG (SEQ ID NO: 156), the CDR2 sequence of FIDRIATTTIATSVKG (SEQ ID NO: 186), and the CDR3 sequence of PLTAR (SEQ ID NO: 217); v) the CDR1 sequence of SSNAMG (SEQ ID NO: 166), the CDR2 sequence of WVGFIDRIATTT (SEQ ID NO: 196), and the CDR3 sequence of NHPLTA (SEQ ID NO: 228); or vi) the CDR1 sequence of GTSVSSNAMG (SEQ ID NO: 176), the CDR2 sequence of FIDRIATTT (SEQ ID NO: 206), and the CDR3 sequence of PLTAR (SEQ ID NO: 239).

In some embodiments, provided is a therapeutic molecule comprising an agent and a VHH domain that binds to an extracellular domain of pIgR, wherein the VHH domain comprises a CDR1 sequence, a CDR2 sequence, and a CDR3 sequence present in VHH5. Accordingly, in some embodiments, the VHH domain comprises: i) the CDR1 sequence of SYAMG (SEQ ID NO: 4), the CDR2 sequence of AITWNGGTTYYADSVKG (SEQ ID NO: 33), and the CDR3 sequence of DPFNQGY (SEQ ID NO: 64); ii) the CDR1 sequence of GRTFSSY (SEQ ID NO: 13), the CDR2 sequence of TWNGGT (SEQ ID NO: 43) or WNGG (SEQ ID NO: 263), and the CDR3 sequence of DPFNQGY (SEQ ID NO: 75) or PFNQG (SEQ ID NO: 276); iii) the CDR1 sequence of GRTFSSYA (SEQ ID NO: 23), the CDR2 sequence of ITWNGGTT (SEQ ID NO: 53), and the CDR3 sequence of AADPFNQGY (SEQ ID NO: 86); iv) the CDR1 sequence of GRTFSSYAMG (SEQ ID NO: 157), the CDR2 sequence of AITWNGGTTYYADSVKG (SEQ ID NO: 187), and the CDR3 sequence of DPFNQGY (SEQ ID NO: 218); v) the CDR1 sequence of SSYAMG (SEQ ID NO: 167), the CDR2 sequence of FVAAITWNGGTTY (SEQ ID NO: 197), and the CDR3 sequence of AADPFNQG (SEQ ID NO: 229); or vi) the CDR1 sequence of GRTFSSYAMG (SEQ ID NO: 177), the CDR2 sequence of AITWNGGTTY (SEQ ID NO: 207), and the CDR3 sequence of DPFNQGY (SEQ ID NO: 240).

In some embodiments, provided is a therapeutic molecule comprising an agent and a VHH domain that binds to an extracellular domain of pIgR, wherein the VHH domain comprises a CDR1 sequence, a CDR2 sequence, and a CDR3 sequence present in VHH6. Accordingly, in some embodiments, the VHH domain comprises: i) the CDR1 sequence of SDAMG (SEQ ID NO: 5), the CDR2 sequence of FISGGGTTTYADSVKG (SEQ ID NO: 34), and the CDR3 sequence of PLTSR (SEQ ID NO: 65); ii) the CDR1 sequence of GSSVSSD (SEQ ID NO: 14), the CDR2 sequence of SGGGT (SEQ ID NO: 44) or GGG (SEQ ID NO: 264), and the CDR3 sequence of PLTSR (SEQ ID NO: 76) or LTS (SEQ ID NO: 277); iii) the CDR1 sequence of GSSVSSDA (SEQ ID NO: 24), the CDR2 sequence of ISGGGTT (SEQ ID NO: 54), and the CDR3 sequence of NHPLTSR (SEQ ID NO: 87); iv) the CDR1 sequence of GSSVSSDAMG (SEQ ID NO: 158), the CDR2 sequence of FISGGGTTTYADSVKG (SEQ ID NO: 188), and the CDR3 sequence of PLTSR (SEQ ID NO: 219); v) the CDR1 sequence of SSDAMG (SEQ ID NO: 168), the CDR2 sequence of WVAFISGGGTTT (SEQ ID NO: 198), and the CDR3 sequence of NHPLTS (SEQ ID NO: 230); or vi) the CDR1 sequence of GSSVSSDAMG (SEQ ID NO: 178), the CDR2 sequence of FISGGGTTT (SEQ ID NO: 208), and the CDR3 sequence of PLTSR (SEQ ID NO: 241).

In some embodiments, provided is a therapeutic molecule comprising an agent and a VHH domain that binds to an extracellular domain of pIgR, wherein the VHH domain comprises a CDR1 sequence, a CDR2 sequence, and a CDR3 sequence present in VHH7. Accordingly, in some embodiments, the VHH domain comprises: i) the CDR1 sequence of INVMG (SEQ ID NO: 6), the CDR2 sequence of RITGGGSTHYAESVKG (SEQ ID NO: 35), and the CDR3 sequence of MVNPIITAWGTIGVREIPDYDY (SEQ ID NO: 66); ii) the CDR1 sequence of RSIGSIN (SEQ ID NO: 15), the CDR2 sequence of TGGGS (SEQ ID NO: 45) or GGG (SEQ ID NO: 265), and the CDR3 sequence of MVNPIITAWGTIGVREIPDYDY (SEQ ID NO: 77) or VNPIITAWGTIGVREIPDYD (SEQ ID NO: 278); iii) the CDR1 sequence of RSIGSINV (SEQ ID NO: 25), the CDR2 sequence of ITGGGST (SEQ ID NO: 55), and the CDR3 sequence of ASMVNPIITAWGTIGVREIPDYDY (SEQ ID NO: 88); iv) the CDR1 sequence of RSIGSINVMG (SEQ ID NO: 159), the CDR2 sequence of RITGGGSTHYAESVKG (SEQ ID NO: 189), and the CDR3 sequence of MVNPIITAWGTIGVREIPDYDY (SEQ ID NO: 220); v) the CDR1 sequence of SINVMG (SEQ ID NO: 169), the CDR2 sequence of LVARITGGGSTH (SEQ ID NO: 199), and the CDR3 sequence of ASMVNPIITAWGTIGVREIPDYD (SEQ ID NO: 231); or vi) the CDR1 sequence of RSIGSINVMG (SEQ ID NO: 179), the CDR2 sequence of RITGGGSTH (SEQ ID NO: 209), and the CDR3 sequence of MVNPIITAWGTIGVREIPDYDY (SEQ ID NO: 242).

In some embodiments, provided is a therapeutic molecule comprising an agent and a VHH domain that binds to an extracellular domain of pIgR, wherein the VHH domain comprises a CDR1 sequence, a CDR2 sequence, and a CDR3 sequence present in VHH9. Accordingly, in some embodiments, the VHH domain comprises: i) the CDR1 sequence of TYRMG (SEQ ID NO: 7), the CDR2 sequence of AISWSGGSTTYADPVKG (SEQ ID NO: 36), and the CDR3 sequence of DQRGY (SEQ ID NO: 67) or QRGY (SEQ ID NO: 271); ii) the CDR1 sequence of GRTFSTY (SEQ ID NO: 16), the CDR2 sequence of SWSGGS (SEQ ID NO: 46) or WSGG (SEQ ID NO: 266), and the CDR3 sequence of DQRGY (SEQ ID NO: 78) or RG (SEQ ID NO: 279); iii) the CDR1 sequence of GRTFSTYR (SEQ ID NO: 26), the CDR2 sequence of ISWSGGST (SEQ ID NO: 56), and the CDR3 sequence of NDQRGY (SEQ ID NO: 89); iv) the CDR1 sequence of GRTFSTYRMG (SEQ ID NO: 160), the CDR2 sequence of AISWSGGSTTYADPVKG (SEQ ID NO: 190), and the CDR3 sequence of QRGY (SEQ ID NO: 221); v) the CDR1 sequence of STYRMG (SEQ ID NO: 170), the CDR2 sequence of FVAAISWSGGSTT (SEQ ID NO: 200), and the CDR3 sequence of NDQRG (SEQ ID NO: 232); or vi) the CDR1 sequence of GRTFSTYRMG (SEQ ID NO: 180), the CDR2 sequence of AISWSGGSTT (SEQ ID NO: 210), and the CDR3 sequence of QRGY (SEQ ID NO: 243).

In some embodiments, provided is a therapeutic molecule comprising an agent and a VHH domain that binds to an extracellular domain of pIgR, wherein the VHH domain comprises a CDR1 sequence, a CDR2 sequence, and a CDR3 sequence present in VHH10. Accordingly, in some embodiments, the VHH domain comprises: i) the CDR1 sequence of RYAMG (SEQ ID NO: 8), the CDR2 sequence of AISWSGSSAGYGDSVKG (SEQ ID NO: 37), and the CDR3 sequence of DPFNQGY (SEQ ID NO: 68); ii) the CDR1 sequence of GFTFTRY (SEQ ID NO: 17), the CDR2 sequence of SWSGSS (SEQ ID NO: 47) or WSGS (SEQ ID NO: 267), and the CDR3 sequence of DPFNQGY (SEQ ID NO: 79) or PFNQG (SEQ ID NO: 280); iii) the CDR1 sequence of GFTFTRYA (SEQ ID NO: 27), the CDR2 sequence of ISWSGSSA (SEQ ID NO: 57), and the CDR3 sequence of AADPFNQGY (SEQ ID NO: 90); iv) the CDR1 sequence of GFTFTRYAMG (SEQ ID NO: 161), the CDR2 sequence of AISWSGSSAGYGDSVKG (SEQ ID NO: 191), and the CDR3 sequence of DPFNQGY (SEQ ID NO: 222); v) the CDR1 sequence of TRYAMG (SEQ ID NO: 171), the CDR2 sequence of FVAAISWSGSSAG (SEQ ID NO: 201), and the CDR3 sequence of AADPFNQG (SEQ ID NO: 233); or vi) the CDR1 sequence of GFTFTRYAMG (SEQ ID NO: 181), the CDR2 sequence of AISWSGSSAG (SEQ ID NO: 211), and the CDR3 sequence of DPFNQGY (SEQ ID NO: 244).

In some embodiments, provided is a therapeutic molecule comprising an agent and a VHH domain that binds to an extracellular domain of pIgR, wherein the VHH domain comprises a CDR1 sequence, a CDR2 sequence, and a CDR3 sequence present in VHH11. Accordingly, in some embodiments, the VHH domain comprises: i) the CDR1 sequence of FTTYRMG (SEQ ID NO: 258) or TYRMG (SEQ ID NO: 259), the CDR2 sequence of AIRWSGGRTLYADSVKG (SEQ ID NO: 38), and the CDR3 sequence of DLAEYSGTYSSPADSPAGYDY (SEQ ID NO: 69); ii) the CDR1 sequence of GRTFTTY (SEQ ID NO: 18), the CDR2 sequence of RWSGGR (SEQ ID NO: 48) or WSGG (SEQ ID NO: 268), and the CDR3 sequence of DLAEYSGTYSSPADSPAGYDY (SEQ ID NO: 80) or LAEYSGTYSSPADSPAGYD (SEQ ID NO: 281); iii) the CDR1 sequence of GRTFTTYR (SEQ ID NO: 28), the CDR2 sequence of IRWSGGRT (SEQ ID NO: 58), and the CDR3 sequence of AADLAEYSGTYSSPADSPAGYDY (SEQ ID NO: 91); iv) the CDR1 sequence of GRTFTTYRMG (SEQ ID NO: 162), the CDR2 sequence of AIRWSGGRTLYADSVKG (SEQ ID NO: 192), and the CDR3 sequence of DLAEYSGTYSSPADSPAGYDY (SEQ ID NO: 223); v) the CDR1 sequence of TTYRMG (SEQ ID NO: 172), the CDR2 sequence of FVAAIRWSGGRTL (SEQ ID NO: 202), and the CDR3 sequence of AADLAEYSGTYSSPADSPAGYD (SEQ ID NO: 234); or vi) the CDR1 sequence of GRTFTTYRMG (SEQ ID NO: 182), the CDR2 sequence of AIRWSGGRTL (SEQ ID NO: 212), and the CDR3 sequence of DLAEYSGTYSSPADSPAGYDY (SEQ ID NO: 245).

In some embodiments, provided is a therapeutic molecule comprising an agent and a VHH domain that binds to an extracellular domain of pIgR, wherein the VHH domain comprises a CDR1 sequence, a CDR2 sequence, and a CDR3 sequence present in VHH12. Accordingly, in some embodiments, the VHH domain comprises: i) the CDR1 sequence of FNTYAMG (SEQ ID NO: 9), the CDR2 sequence of SITWNGGSTSYADSVKG (SEQ ID NO: 39), and the CDR3 sequence of ARYYVSGTYFPANY (SEQ ID NO: 70); ii) the CDR1 sequence of GRTLSFNTY (SEQ ID NO: 19), the CDR2 sequence of TWNGGS (SEQ ID NO: 49) or WNGG (SEQ ID NO: 269), and the CDR3 sequence of ARYYVSGTYFPANY (SEQ ID NO: 81) or RYYVSGTYFPAN (SEQ ID NO: 282); iii) the CDR1 sequence of GRTLSFNTYA (SEQ ID NO: 29), the CDR2 sequence of ITWNGGST (SEQ ID NO: 59), and the CDR3 sequence of AAARYYVSGTYFPANY (SEQ ID NO: 92); iv) the CDR1 sequence of GRTLSFNTYAMG (SEQ ID NO: 163), the CDR2 sequence of SITWNGGSTSYADSVKG (SEQ ID NO: 193), and the CDR3 sequence of ARYYVSGTYFPANY (SEQ ID NO: 224); v) the CDR1 sequence of SFNTYAMG (SEQ ID NO: 173), the CDR2 sequence of FVASITWNGGSTS (SEQ ID NO: 203), and the CDR3 sequence of AAARYYVSGTYFPAN (SEQ ID NO: 235); or vi) the CDR1 sequence of GRTLSFNTYAMG (SEQ ID NO: 183), the CDR2 sequence of SITWNGGSTS (SEQ ID NO: 213), and the CDR3 sequence of ARYYVSGTYFPANY (SEQ ID NO: 246).

In some embodiments, the VHH domain comprises a framework derived from the framework of any of the VHH domains comprising the sequences of

(SEQ ID NO: 93)
QVQLVESGGGLVQAGGSLKLACAAPGLTFSSYRMGWFRQAPGQERE
FVAAIDWNGRGTYYRYYADSVKGRSTISRDNAKNTMYLQMNSLKPE
DTAVYYCAAGSIDLNWYGGMDYWGQGTQVTVSS,
(SEQ ID NO: 94)
EVQVVESGGGLVQAGGSLKLACAAPGLTFSSYRMGWFRQAPGQERE
FVAAIDWNGRGTYYRYYADSVKGRSTISRDNAKNTVYLQMNSLKPE
DTAVYYCAATTVLTDPRVLNEYATWGQGTQVTVSS,
(SEQ ID NO: 95)
QLQLVESGGGLVQPGGSLRLSCAASGSIFSINVMGWYRQAPGKQRE
LVARINGGGITHYAESVKGRFTISRDNAKNTVYLQMNSLKPEDTAA
YYCKADVFGSSGYVETYWGQGTQVTVSS,
(SEQ ID NO: 96)
EVQVVESGGGLVQAGGSLRLSCAVSGTSVSSNAMGWYRQAPGKQRE
WVGFIDRIATTTIATSVKGRFAITRDNAKNTVYLQMSGLKPEDTAV
YYCNHPLTARWGQGTQVTVSS,
(SEQ ID NO: 97)
QVQLVESGGGLVQAGGSLRLSCAASGRTFSSYAMGWFRQAPGKERE
FVAAITWNGGTTYYADSVKGRFTISRDNAKNTVYLQMNSLKPEDTA
VYYCAADPFNQGYWGQGTQVTVSS,
(SEQ ID NO: 98)
EVQLVESGGGLVQAGGSLRLSCAVSGSSVSSDAMGWYRQAPGNQRA
WVAFISGGGTTTYADSVKGRFTISRDNTKNTVYLHMNSLKPEDTAV
YYCNHPLTSRWGQGTQVTVSS,
(SEQ ID NO: 99)
EVQVVESGGGLVQAGGSLRLACVASRSIGSINVMGWYRQAPGKQRD
LVARITGGGSTHYAESVKGRFTISRDNAKNTVYLQMNSLEPEDTAV
YYCASMVNPIITAWGTIGVREIPDYDYWGQGTQVTVSS,
(SEQ ID NO: 100)
QVQLVESGGGLVQAGGSLRLSCAVSGRTFSTYRMGWFRQAPGKERS
FVAAISWSGGSTTYADPVKGRFTISRDNAKNTVYLRMNSLKPEDTA
VYYCNDQRGYWGQGTLVTVSS,
(SEQ ID NO: 101)
EVQVVESGGGLVQAGGSLRLSCAASGFTFTRYAMGWFRQAPGKERS
FVAAISWSGSSAGYGDSVKGRFTISRDNAKNTLYLQMNSLKPEDTA
VYYCAADPFNQGYWGQGTQVTVSS,
(SEQ ID NO: 102)
EVQVVESGGGLVQAGGSLRLSCAASGRTFTTYRMGWFRQAPGKERE
FVAAIRWSGGRTLYADSVKGRFTISRDNAKNTAYLQMNNLRPEDTA
VYYCAADLAEYSGTYSSPADSPAGYDYWGQGTQVTVSS,
or
(SEQ ID NO: 103)
QVQLVETGGGLVQAGDSLRLSCAASGRTLSFNTYAMGWFRQAPGKE
REFVASITWNGGSTSYADSVKGRFTITRDNAKNTATLRMNSLQPDD
TAVYYCAAARYYVSGTYFPANYWGQGTQVTVSS.

In some embodiments, the VHH domain comprises a framework comprising sequence having at least 75%, 80%, 85%, 90%, 95%, 96%, 97%, 98%, 99%, or 100% sequence identity with the sequence of

(SEQ ID NO: 93)
QVQLVESGGGLVQAGGSLKLACAAPGLTFSSYRMGWFRQAPGQERE
FVAAIDWNGRGTYYRYYADSVKGRSTISRDNAKNTMYLQMNSLKPE
DTAVYYCAAGSIDLNWYGGMDYWGQGTQVTVSS,
(SEQ ID NO: 94)
EVQVVESGGGLVQAGGSLKLACAAPGLTFSSYRMGWFRQAPGQERE
FVAAIDWNGRGTYYRYYADSVKGRSTISRDNAKNTVYLQMNSLKPE
DTAVYYCAATTVLTDPRVLNEYATWGQGTQVTVSS,
(SEQ ID NO: 95)
QLQLVESGGGLVQPGGSLRLSCAASGSIFSINVMGWYRQAPGKQRE
LVARINGGGITHYAESVKGRFTISRDNAKNTVYLQMNSLKPEDTAA
YYCKADVFGSSGYVETYWGQGTQVTVSS,
(SEQ ID NO: 96)
EVQVVESGGGLVQAGGSLRLSCAVSGTSVSSNAMGWYRQAPGKQRE
WVGFIDRIATTTIATSVKGRFAITRDNAKNTVYLQMSGLKPEDTAV
YYCNHPLTARWGQGTQVTVSS,
(SEQ ID NO: 97)
QVQLVESGGGLVQAGGSLRLSCAASGRTFSSYAMGWFRQAPGKERE
FVAAITWNGGTTYYADSVKGRFTISRDNAKNTVYLQMNSLKPEDTA
VYYCAADPFNQGYWGQGTQVTVSS,
(SEQ ID NO: 98)
EVQLVESGGGLVQAGGSLRLSCAVSGSSVSSDAMGWYRQAPGNQRA
WVAFISGGGTTTYADSVKGRFTISRDNTKNTVYLHMNSLKPEDTAV
YYCNHPLTSRWGQGTQVTVSS,
(SEQ ID NO: 99)
EVQVVESGGGLVQAGGSLRLACVASRSIGSINVMGWYRQAPGKQRD
LVARITGGGSTHYAESVKGRFTISRDNAKNTVYLQMNSLEPEDTAV
YYCASMVNPIITAWGTIGVREIPDYDYWGQGTQVTVSS,
(SEQ ID NO: 100)
QVQLVESGGGLVQAGGSLRLSCAVSGRTFSTYRMGWFRQAPGKERS
FVAAISWSGGSTTYADPVKGRFTISRDNAKNTVYLRMNSLKPEDTA
VYYCNDQRGYWGQGTLVTVSS,
(SEQ ID NO: 101)
EVQVVESGGGLVQAGGSLRLSCAASGFTFTRYAMGWFRQAPGKERS
FVAAISWSGSSAGYGDSVKGRFTISRDNAKNTLYLQMNSLKPEDTA
VYYCAADPFNQGYWGQGTQVTVSS,
(SEQ ID NO: 102)
EVQVVESGGGLVQAGGSLRLSCAASGRTFTTYRMGWFRQAPGKERE
FVAAIRWSGGRTLYADSVKGRFTISRDNAKNTAYLQMNNLRPEDTA
VYYCAADLAEYSGTYSSPADSPAGYDYWGQGTQVTVSS,
or
(SEQ ID NO: 103)
QVQLVETGGGLVQAGDSLRLSCAASGRTLSFNTYAMGWFRQAPGKE
REFVASITWNGGSTSYADSVKGRFTITRDNAKNTATLRMNSLQPDD
TAVYYCAAARYYVSGTYFPANYWGQGTQVTVSS.

In some embodiments, the VHH domain is comprised of a sequence having at least 75%, 80%, 85%, 90%, 95%, 96%, 97%, 98%, 99%, or 100% sequence identity with the sequence of

(SEQ ID NO: 93)
QVQLVESGGGLVQAGGSLKLACAAPGLTFSSYRMGWFRQAPGQEREFVA
AIDWNGRGTYYRYYADSVKGRSTISRDNAKNTMYLQMNSLKPEDTAVYY
CAAGSIDLNWYGGMDYWGQGTQVTVSS,
(SEQ ID NO: 94)
EVQVVESGGGLVQAGGSLKLACAAPGLTFSSYRMGWFRQAPGQEREFVA
AIDWNGRGTYYRYYADSVKGRSTISRDNAKNTVYLQMNSLKPEDTAVYY
CAATTVLTDPRVLNEYATWGQGTQVTVSS,
(SEQ ID NO: 95)
EVQVVESGGGLVQAGGSLRLSCAVSGTSVSSNAMGWYRQAPGKQREWVG
FIDRIATTTIATSVKGRFAITRDNAKNTVYLQMSGLKPEDTAVYYCNHP
LTARWGQGTQVTVSS,
(SEQ ID NO: 96)
QVQLVESGGGLVQAGGSLRLSCAASGRTFSSYAMGWFRQAPGKEREFVA
AITWNGGTTYYADSVKGRFTISRDNAKNTVYLQMNSLKPEDTAVYYCAA
DPFNQGYWGQGTQVTVSS,
(SEQ ID NO: 97)
EVQLVESGGGLVQAGGSLRLSCAVSGSSVSSDAMGWYRQAPGNQRAWVA
FISGGGTTTYADSVKGRFTISRDNTKNTVYLHMNSLKPEDTAVYYCNHP
LTSRWGQGTQVTVSS,
(SEQ ID NO: 98)
EVQVVESGGGLVQAGGSLRLACVASRSIGSINVMGWYRQAPGKQRDLVA
RITGGGSTHYAESVKGRFTISRDNAKNTVYLQMNSLEPEDTAVYYCASM
VNPIITAWGTIGVREIPDYDYWGQGTQVTVSS,
(SEQ ID NO: 99)
QVQLVESGGGLVQAGGSLRLSCAVSGRTFSTYRMGWFRQAPGKERSFVA
AISWSGGSTTYADPVKGRFTISRDNAKNTVYLRMNSLKPEDTAVYYCND
QRGYWGQGTLVTVSS,
(SEQ ID NO: 100)
EVQVVESGGGLVQAGGSLRLSCAASGFTFTRYAMGWFRQAPGKERSFVA
AISWSGSSAGYGDSVKGRFTISRDNAKNTLYLQMNSLKPEDTAVYYCAA
DPFNQGYWGQGTQVTVSS,
(SEQ ID NO: 101)
EVQVVESGGGLVQAGGSLRLSCAASGRTFTTYRMGWFRQAPGKEREFVA
AIRWSGGRTLYADSVKGRFTISRDNAKNTAYLQMNNLRPEDTAVYYCAA
DLAEYSGTYSSPADSPAGYDYWGQGTQVTVSS,
or
(SEQ ID NO: 102)
QVQLVETGGGLVQAGDSLRLSCAASGRTLSFNTYAMGWFRQAPGKEREF
VASITWNGGSTSYADSVKGRFTITRDNAKNTATLRMNSLQPDDTAVYYC
AAARYYVSGTYFPANYWGQGTQVTVSS.

In some embodiments, the agent is an antibody or fragment thereof, a peptide, a vaccine, a small molecule, a polynucleotide, a radioisotope, a toxin, an enzyme, an anticoagulant, a hormone, a cytokine, an anti-inflammatory molecule, an RNAi, an mRNA, a self-replicating RNA, an antibiotic, or an antibody-antibiotic conjugate. In some embodiments, the agent is an antibiotic. In some embodiments, the VHH domain is genetically fused or chemically conjugated to the agent. In some embodiments, the therapeutic molecule further comprises a linker between the VHH domain and the agent. The linker may be a polypeptide. The linker may be a flexible linker comprising a sequence selected from the group consisting of EPKTPKPQPQPQLQPQPNPTTESKSPK (SEQ ID NO: 130), (EAAAK)n (SEQ ID NO: 147), (GGGGS)n (SEQ ID NO: 148) and (GGGS)n (SEQ ID NO: 149), wherein n is an integer from 1 to 20. In some embodiments, the VHH domain is chemically-conjugated to the agent. In some embodiments, the VHH domain is non-covalently bound to the agent.

In another aspect is provided a pharmaceutical composition comprising any of the above therapeutic molecules and a pharmaceutically acceptable carrier.

In another aspect is provided a method of delivering a therapeutic molecule to a mucosal lumen of a subject, the method comprising administering to the subject an effective amount of any of the above therapeutic molecules. In some embodiments, the therapeutic molecule is delivered to the mucosal lumen via forward transcytosis from the basolateral surface of a mucosal epithelial cell to the apical surface of the mucosal epithelial cell. In some embodiments, the mucosal epithelial cell is at or adjacent to the mucosal lumen. In some embodiments, the mucosal lumen is in the lung or in the gastrointestinal tract of the subject. In some embodiments, the mucosal epithelial cell is a cancer cell (e.g., a lung cancer cell, an esophageal cancer cell, a stomach cancer cell, a duodenal cancer cell, a liver cancer cell, a bladder cancer cell, a sinus cancer cell, a nasal cavity cancer cell, an endometrial cancer cell or a colorectal cancer cell.) In some embodiments, the cell is in a subject.

In another aspect is provided a method of delivering a therapeutic molecule to an organ of a subject, the method comprising administering to the subject any of the above therapeutic molecules. In some embodiments, the organ is selected from the group consisting of gastrointestinal track, small intestine, large intestine, stomach, esophagus, salivary gland, lung, vagina, uterus, and lacrimal gland. In some embodiments, the organ is a lung. In some embodiments, the agent is an antibody or fragment thereof, a peptide, a vaccine, a small molecule, a polynucleotide, a molecule comprising a radioactive isotope, a toxin, an enzyme, an anticoagulant, a hormone, a cytokine, an anti-inflammatory molecule, an RNAi, an antibiotic, or an antibody-antibiotic conjugate. In some embodiments, the agent is an antibiotic (e.g., a macrolide antibiotic, a fluoroquinolone, a tetracycline, amoxicillin, ceftriaxone, penicillin G, linezolid, moxifloxacin, and azithromycin.) In various embodiments, the therapeutic molecule is administered to the bloodstream of the subject. In some embodiments, the molecule is administered intravenously or subcutaneously.

In another aspect is provided a method of delivering a therapeutic molecule into systemic circulation in a subject, the method comprising administering to the subject the therapeutic molecule of any of the above. In some embodiments, the therapeutic molecule is delivered into the systemic circulation via reverse transcytosis from the apical surface of an epithelial cell to the basolateral surface of the epithelial cell. In some embodiments, the therapeutic molecule is delivered by oral delivery, buccal delivery, nasal delivery or inhalation delivery. In some embodiments, the agent is a peptide, an antibody or fragment thereof or a vaccine.

In another aspect is provided a method of delivering a therapeutic molecule into Lamina propria of a subject, the method comprising administering to the subject the therapeutic molecule of any of the above. In some embodiments, the therapeutic molecule is delivered into the Lamina propria via reverse transcytosis from the apical surface of an epithelial cell to the basolateral surface of the epithelial cell. In some embodiments, the therapeutic molecule is delivered by oral delivery or buccal delivery. In some embodiments, the agent is a peptide or an antibody or fragment thereof.

In another aspect is provided a method of increasing the rate of pIgR-mediated transcytosis across an epithelial cell comprising contacting the cell with (i) a VHH domain that binds to an extracellular domain 1, an extracellular domain 2, an extracellular domain 1-2, an extracellular domain 3, an extracellular domain 2-3, an extracellular domain 4-5, or an extracellular domain 5 of pIgR or (ii) a therapeutic molecule comprising an agent and the VHH domain. In some embodiments, the transcytosis is forward transcytosis. In some embodiments, the transcytosis is reverse transcytosis.

In another aspect is provided a method of modulating a function of pIgR in a cell comprising contacting the cell with an effective amount of (i) a VHH domain that binds to an extracellular domain 1, an extracellular domain 2, an extracellular domain 1-2, an extracellular domain 3, an extracellular domain 2-3, an extracellular domain 4-5, or an extracellular domain 5 of pIgR or (ii) a therapeutic molecule comprising an agent and the VHH domain. In some embodiments, the modulating the function of pIgR in the cell is activating said function of pIgR in said cell. In some embodiments, the modulating the function of pIgR in the cell is inhibiting said function of pIgR in said cell.

In another aspect is provided a method of delivery to a pIgR-expressing cell comprising contacting the cell with a VHH domain or a therapeutic molecule, wherein the VHH domain binds to an extracellular domain 1, an extracellular domain 2, an extracellular domain 1-2, an extracellular domain 3, an extracellular domain 2-3, an extracellular domain 4-5, or an extracellular domain 5 of pIgR, and wherein the a therapeutic molecule comprises an agent and the VHH domain. In some embodiments, the method of delivery is oral delivery, buccal delivery, nasal delivery or inhalation delivery.

In some embodiments, a method described above comprises a VHH domain that competes with IgA binding to the pIgR. In some embodiments, a method described above comprises a VHH domain that promotes IgA binding to the pIgR. In some embodiments, the K_{D} of the binding of the VHH domain to pIgR is from about 4 to about 525 nM. In some embodiments, the K_{D} of the binding of the VHH domain to pIgR is less than about 50 nM. In some embodiments, the K_{D} of the binding of the VHH domain to pIgR is from about 4 to about 34 nM. In some embodiments, the Tₘ of the VHH domain is from about 53 to about 77° C. In some embodiments, the Tₘ of the VHH domain is from 53.9 to 76.4° C.

In another aspect, provided herein is a method to diagnose a disease or condition, the method comprising administering to the subject (i) a VHH domain that binds to an extracellular domain 1, an extracellular domain 2, an extracellular domain 1-2, an extracellular domain 3, an extracellular domain 2-3, an extracellular domain 4-5, or an extracellular domain 5 of pIgR, or (ii) a therapeutic molecule comprising an agent and the VHH domain, to the subject, the method comprising detecting the amount of VHH domain in a tissue of the subject, wherein the tissue comprises a diseased cell, and comparing the amount of VHH domain in the tissue of the subject with a reference amount of VHH domain in the tissue of a comparable healthy subject. In some embodiments, the tissue comprises a mucosal cell. In some embodiments, the tissue comprises a mucosal lumen. In some embodiments, the VHH domain competes with IgA binding to the pIgR. In some embodiments, the VHH domain promotes IgA binding to the pIgR.

In some embodiments, a method described above comprises a VHH domain, wherein the K_{D} of the binding of the VHH domain to pIgR is from about 4 to about 525 nM. In some embodiments, the K_{D} of the binding of the VHH domain to pIgR is less than about 50 nM. In some embodiments, the K_{D} of the binding of the VHH domain to pIgR is from about 4 to about 34 nM. In some embodiments, the Tₘ of the VHH domain is from about 53 to about 77° C. In some embodiments, the Tₘ of the VHH domain is from 53.9 to 76.4° C.

In some embodiments, a method described above comprises a therapeutic molecule that comprise VHH domain and an agent, wherein the agent is an antibody or fragment thereof, a peptide, a vaccine, a small molecule, a polynucleotide, a radioisotope, a toxin, an enzyme, an anticoagulant, a hormone, a cytokine, an anti-inflammatory molecule, an RNAi, an mRNA, a self-replicating RNA, an antibiotic, or an antibody-antibiotic conjugate. In some embodiments, the agent is an antibiotic. In some embodiments, the VHH domain is genetically fused or chemically conjugated to the agent. In some embodiments, a linker is between the VHH domain and the agent. In some embodiments, the linker is a polypeptide. In some embodiments, the linker is a flexible linker comprising a sequence selected from the group consisting of EPKTPKPQPQPQLQPQPNPTTESKSPK (SEQ ID NO: 130), (EAAAK)n (SEQ ID NO: 147), (GGGGS)n (SEQ ID NO: 148) and (GGGS)n (SEQ ID NO: 149), wherein n is an integer from 1 to 20.

In some embodiments, a method described above comprises a therapeutic molecule that comprise VHH domain and an agent, wherein the VHH domain is chemically-conjugated to the agent. In some embodiments, the VHH domain is non-covalently bound to the agent. In some embodiments, the VHH domain comprises a radioisotope. In some embodiments, the radioisotope is zirconium-89.

In various embodiments, a method to diagnose a disease or condition described above comprises a method wherein the disease is lung cancer, and wherein the tissue is lung. In various embodiments, the disease is endometrial cancer, and wherein the tissue is the uterus. In various embodiments, the disease is colon cancer, and wherein the tissue is the colon. In various embodiments, the disease is an inflammatory disease, and wherein the tissue is Lamina propria. In some embodiments, the inflammatory disease is inflammatory bowel disease, Crohn's disease or ulcerative colitis. In various embodiments, the diseased cell expresses an antigen, and wherein the therapeutic molecule is coupled to an antibody that specifically recognizes the antigen. In various embodiments, the antigen is specific to the diseased cell.

In some embodiments, a method described above comprises a VHH domain that binds to an extracellular domain of pIgR. In some embodiment, the VHH domain binds to an extracellular domain 1 of pIgR. In some embodiments, the VHH domain binds to an extracellular domain 2 of pIgR. In some embodiments, the VHH domain binds to an extracellular domain 1-2 of pIgR. In some embodiments, the VHH domain binds to an extracellular domain 3 of pIgR. In some embodiments, the VHH domain binds to an extracellular domain 2-3 of pIgR. In some embodiments, the VHH domain binds to an extracellular domain 4-5 of pIgR. In some embodiments, the VHH domain binds to an extracellular domain 5 of pIgR. In some embodiments, the pIgR is human pIgR. In some embodiments, the pIgR is mouse pIgR. In some embodiments, the VHH domain does not detectably bind to the amino acid sequence of

(SEQ ID NO: 143)
EKAVADTRDQADGSRASVDSGSSEEQGGSSR
(SEQ ID NO: 144)
EREIQNVGDQAQENRASGDAGSADGQSRSSSSK,
or
(SEQ ID NO: 145)
EREIQNVRDQAQENRASGDAGSADGQSRSSSSK.

In some embodiments, a method described above comprises a VHH domain that comprises a CDR1 sequence of SYRMG (SEQ ID NO: 1), INVMG (SEQ ID NO: 2), SNAMG (SEQ ID NO: 3), SYAMG (SEQ ID NO: 4), SDAMG (SEQ ID NO: 5), INVMG (SEQ ID NO: 6), TYRMG (SEQ ID NO: 7), RYAMG (SEQ ID NO: 8), FTTYRMG (SEQ ID NO: 258), TYRMG (SEQ ID NO: 259), FNTYAMG (SEQ ID NO: 9), GLTFSSY (SEQ ID NO: 10), GSIFSIN (SEQ ID NO: 11), GTSVSSN (SEQ ID NO: 12), GRTFSSY (SEQ ID NO: 13), GSSVSSD (SEQ ID NO: 14), RSIGSIN (SEQ ID NO: 15), GRTFSTY (SEQ ID NO: 16), GFTFTRY (SEQ ID NO: 17), GRTFTTY (SEQ ID NO: 18), GRTLSFNTY (SEQ ID NO: 19), GLTFSSYR (SEQ ID NO: 20), GSIFSINV (SEQ ID NO: 21), GTSVSSNA (SEQ ID NO: 22), GRTFSSYA (SEQ ID NO: 23), GSSVSSDA (SEQ ID NO: 24), RSIGSINV (SEQ ID NO: 25), GRTFSTYR (SEQ ID NO: 26), GFTFTRYA (SEQ ID NO: 27), GRTFTTYR (SEQ ID NO: 28), GRTLSFNTYA (SEQ ID NO: 29), GLTFSSYRMG (SEQ ID NO: 154), GSIFSINVMG (SEQ ID NO: 155), GTSVSSNAMG (SEQ ID NO: 156), GRTFSSYAMG (SEQ ID NO: 157), GSSVSSDAMG (SEQ ID NO: 158), RSIGSINVMG (SEQ ID NO: 159), GRTFSTYRMG (SEQ ID NO: 160), GFTFTRYAMG (SEQ ID NO: 161), GRTFTTYRMG (SEQ ID NO: 162), GRTLSFNTYAMG (SEQ ID NO: 163), SSYRMG (SEQ ID NO: 164), SINVMG (SEQ ID NO: 165), SSNAMG (SEQ ID NO: 166), SSYAMG (SEQ ID NO: 167), SSDAMG (SEQ ID NO: 168), SINVMG (SEQ ID NO: 169), STYRMG (SEQ ID NO: 170), TRYAMG (SEQ ID NO: 171), TTYRMG (SEQ ID NO: 172), SFNTYAMG (SEQ ID NO: 173), GLTFSSYRMG (SEQ ID NO: 174), GSIFSINVMG (SEQ ID NO: 175), GTSVSSNAMG (SEQ ID NO: 176), GRTFSSYAMG (SEQ ID NO: 177), GSSVSSDAMG (SEQ ID NO: 178), RSIGSINVMG (SEQ ID NO: 179), GRTFSTYRMG (SEQ ID NO: 180), GFTFTRYAMG (SEQ ID NO: 181), GRTFTTYRMG (SEQ ID NO: 182), or GRTLSFNTYAMG (SEQ ID NO: 183).

In some embodiments, a method described above comprises a VHH domain that comprises a CDR2 sequence of AIDWNGRGTYYRYYADSVKG (SEQ ID NO: 30), RINGGGITHYAESVKG (SEQ ID NO: 31), FIDRIATTTIATSVKG (SEQ ID NO: 32), AITWNGGTTYYADSVKG (SEQ ID NO: 33), FISGGGTTTYADSVKG (SEQ ID NO: 34), RITGGGSTHYAESVKG (SEQ ID NO: 35), AISWSGGSTTYADPVKG (SEQ ID NO: 36), AISWSGSSAGYGDSVKG (SEQ ID NO: 37), AIRWSGGRTLYADSVKG (SEQ ID NO: 38), SITWNGGSTSYADSVKG (SEQ ID NO: 39), DWNGRGTYY (SEQ ID NO: 40), WNGRGTY (SEQ ID NO: 260), NGGGI (SEQ ID NO: 41), GGG (SEQ ID NO: 261), DRIAT (SEQ ID NO: 42), RIA (SEQ ID NO: 262), TWNGGT (SEQ ID NO: 43), WNGG (SEQ ID NO: 263), SGGGT (SEQ ID NO: 44), GGG (SEQ ID NO: 264), TGGGS (SEQ ID NO: 45), GGG (SEQ ID NO: 265), SWSGGS (SEQ ID NO: 46), WSGG (SEQ ID NO: 266), SWSGSS (SEQ ID NO: 47), WSGS (SEQ ID NO: 267), RWSGGR (SEQ ID NO: 48), WSGG (SEQ ID NO: 268), TWNGGS (SEQ ID NO: 49), WNGG (SEQ ID NO: 269), IDWNGRGTYY (SEQ ID NO: 50), IDWNGRGTYYR (SEQ ID NO: 270), INGGGIT (SEQ ID NO: 51), IDRIATT (SEQ ID NO: 52), ITWNGGTT (SEQ ID NO: 53), ISGGGTT (SEQ ID NO: 54), ITGGGST (SEQ ID NO: 55), ISWSGGST (SEQ ID NO: 56), ISWSGSSA (SEQ ID NO: 57), IRWSGGRT (SEQ ID NO: 58), ITWNGGST (SEQ ID NO: 59), AIDWNGRGTYYRYYADSVKG (SEQ ID NO: 184), RINGGGITHYAESVKG (SEQ ID NO: 185), FIDRIATTTIATSVKG (SEQ ID NO: 186), AITWNGGTTYYADSVKG (SEQ ID NO: 187), FISGGGTTTYADSVKG (SEQ ID NO: 188), RITGGGSTHYAESVKG (SEQ ID NO: 189), AISWSGGSTTYADPVKG (SEQ ID NO: 190), AISWSGSSAGYGDSVKG (SEQ ID NO: 191), AIRWSGGRTLYADSVKG (SEQ ID NO: 192), SITWNGGSTSYADSVKG (SEQ ID NO: 193), FVAAIDWNGRGTYYRY (SEQ ID NO: 194), LVARINGGGITH (SEQ ID NO: 195), WVGFIDRIATTT (SEQ ID NO: 196), FVAAITWNGGTTY (SEQ ID NO: 197), WVAFISGGGTTT (SEQ ID NO: 198), LVARITGGGSTH (SEQ ID NO: 199), FVAAISWSGGSTT (SEQ ID NO: 200), FVAAISWSGSSAG (SEQ ID NO: 201), FVAAIRWSGGRTL (SEQ ID NO: 202), FVASITWNGGSTS (SEQ ID NO: 203), AIDWNGRGTYYRY (SEQ ID NO: 204), RINGGGITH (SEQ ID NO: 205), FIDRIATTT (SEQ ID NO: 206), AITWNGGTTY (SEQ ID NO: 207), FISGGGTTT (SEQ ID NO: 208), RITGGGSTH (SEQ ID NO: 209), AISWSGGSTT (SEQ ID NO: 210), AISWSGSSAG (SEQ ID NO: 211), AIRWSGGRTL (SEQ ID NO: 212), or SITWNGGSTS (SEQ ID NO: 213).

In some embodiments, a method described above comprises a VHH domain that comprises a CDR3 sequence of GSIDLNWYGGMDY (SEQ ID NO: 60), TTVLTDPRVLNEYAT (SEQ ID NO: 61), DVFGSSGYVETY (SEQ ID NO: 62), PLTAR (SEQ ID NO: 63), DPFNQGY (SEQ ID NO: 64), PLTSR (SEQ ID NO: 65), MVNPIITAWGTIGVREIPDYDY (SEQ ID NO: 66), DQRGY (SEQ ID NO: 67), QRGY (SEQ ID NO: 271), DPFNQGY (SEQ ID NO: 68), DLAEYSGTYSSPADSPAGYDY (SEQ ID NO: 69), ARYYVSGTYFPANY (SEQ ID NO: 70), GSIDLNWYGGMDY (SEQ ID NO: 71), SIDLNWYGGMD (SEQ ID NO: 272), TTVLTDPRVLNEYAT (SEQ ID NO: 72), TVLTDPRVLNEYA (SEQ ID NO: 273), DVFGSSGYVETY (SEQ ID NO: 73), VFGSSGYVET (SEQ ID NO: 274), PLTAR (SEQ ID NO: 74), LTA (SEQ ID NO: 275), DPFNQGY (SEQ ID NO: 75), PFNQG (SEQ ID NO: 276), PLTSR (SEQ ID NO: 76), LTS (SEQ ID NO: 277), MVNPIITAWGTIGVREIPDYDY (SEQ ID NO: 77), VNPIITAWGTIGVREIPDYD (SEQ ID NO: 278), DQRGY (SEQ ID NO: 78), RG (SEQ ID NO: 279), DPFNQGY (SEQ ID NO: 79), PFNQG (SEQ ID NO: 280), DLAEYSGTYSSPADSPAGYDY (SEQ ID NO: 80), LAEYSGTYSSPADSPAGYD (SEQ ID NO: 281), ARYYVSGTYFPANY (SEQ ID NO: 81), RYYVSGTYFPAN (SEQ ID NO: 282), CAAGSIDLNWYGGMDY (SEQ ID NO: 82), AAGSIDLNWYGGMDY (SEQ ID NO: 283), CAATTVLTDPRVLNEYAT (SEQ ID NO: 83), AATTVLTDPRVLNEYAT (SEQ ID NO: 284), KADVFGSSGYVETY (SEQ ID NO: 84), NHPLTAR (SEQ ID NO: 85), AADPFNQGY (SEQ ID NO: 86), NHPLTSR (SEQ ID NO: 87), ASMVNPIITAWGTIGVREIPDYDY (SEQ ID NO: 88), NDQRGY (SEQ ID NO: 89), AADPFNQGY (SEQ ID NO: 90), AADLAEYSGTYSSPADSPAGYDY (SEQ ID NO: 91), AAARYYVSGTYFPANY (SEQ ID NO: 92), GSIDLNWYGGMDY (SEQ ID NO: 214), TTVLTDPRVLNEYAT (SEQ ID NO: 215), DVFGSSGYVETY (SEQ ID NO: 216), PLTAR (SEQ ID NO: 217), DPFNQGY (SEQ ID NO: 218), PLTSR (SEQ ID NO: 219), MVNPIITAWGTIGVREIPDYDY (SEQ ID NO: 220), QRGY (SEQ ID NO: 221), DPFNQGY (SEQ ID NO: 222), DLAEYSGTYSSPADSPAGYDY (SEQ ID NO: 223), ARYYVSGTYFPANY (SEQ ID NO: 224), AAGSIDLNWYGGMD (SEQ ID NO: 225), AATTVLTDPRVLNEYA (SEQ ID NO: 226), KADVFGSSGYVET (SEQ ID NO: 227), NHPLTA (SEQ ID NO: 228), AADPFNQG (SEQ ID NO: 229), NHPLTS (SEQ ID NO: 230), ASMVNPIITAWGTIGVREIPDYD (SEQ ID NO: 231), NDQRG (SEQ ID NO: 232), AADPFNQG (SEQ ID NO: 233), AADLAEYSGTYSSPADSPAGYD (SEQ ID NO: 234), AAARYYVSGTYFPAN (SEQ ID NO: 235), GSIDLNWYGGMDY (SEQ ID NO: 236), TTVLTDPRVLNEYAT (SEQ ID NO: 237), DVFGSSGYVETY (SEQ ID NO: 238), PLTAR (SEQ ID NO: 239), DPFNQGY (SEQ ID NO: 240), PLTSR (SEQ ID NO: 241), MVNPIITAWGTIGVREIPDYDY (SEQ ID NO: 242), QRGY (SEQ ID NO: 243), DPFNQGY (SEQ ID NO: 244), DLAEYSGTYSSPADSPAGYDY (SEQ ID NO: 245), or ARYYVSGTYFPANY (SEQ ID NO: 246).

In some embodiments, a method described above comprises a VHH domain that comprises a framework derived from the framework of any of the VHH domains comprising the sequences of

(SEQ ID NO: 93)
QVQLVESGGGLVQAGGSLKLACAAPGLTFSSYRMGWFRQAPGQEREFVA
AIDWNGRGTYYRYYADSVKGRSTISRDNAKNTMYLQMNSLKPEDTAVYY
CAAGSIDLNWYGGMDYWGQGTQVTVSS,
(SEQ ID NO: 94)
EVQVVESGGGLVQAGGSLKLACAAPGLTFSSYRMGWFRQAPGQEREFVA
AIDWNGRGTYYRYYADSVKGRSTISRDNAKNTVYLQMNSLKPEDTAVYY
CAATTVLTDPRVLNEYATWGQGTQVTVSS,
(SEQ ID NO: 95)
EVQVVESGGGLVQAGGSLRLSCAVSGTSVSSNAMGWYRQAPGKQREWVG
FIDRIATTTIATSVKGRFAITRDNAKNTVYLQMSGLKPEDTAVYYCNHP
LTARWGQGTQVTVSS,
(SEQ ID NO: 96)
QVQLVESGGGLVQAGGSLRLSCAASGRTFSSYAMGWFRQAPGKEREFVA
AITWNGGTTYYADSVKGRFTISRDNAKNTVYLQMNSLKPEDTAVYYCAA
DPFNQGYWGQGTQVTVSS,
(SEQ ID NO: 97)
EVQLVESGGGLVQAGGSLRLSCAVSGSSVSSDAMGWYRQAPGNQRAWVA
FISGGGTTTYADSVKGRFTISRDNTKNTVYLHMNSLKPEDTAVYYCNHP
LTSRWGQGTQVTVSS,
(SEQ ID NO: 98)
EVQVVESGGGLVQAGGSLRLACVASRSIGSINVMGWYRQAPGKQRDLVA
RITGGGSTHYAESVKGRFTISRDNAKNTVYLQMNSLEPEDTAVYYCASM
VNPIITAWGTIGVREIPDYDYWGQGTQVTVSS,
(SEQ ID NO: 99)
QVQLVESGGGLVQAGGSLRLSCAVSGRTFSTYRMGWFRQAPGKERSFVA
AISWSGGSTTYADPVKGRFTISRDNAKNTVYLRMNSLKPEDTAVYYCND
QRGYWGQGTLVTVSS,
(SEQ ID NO: 100)
EVQVVESGGGLVQAGGSLRLSCAASGFTFTRYAMGWFRQAPGKERSFVA
AISWSGSSAGYGDSVKGRFTISRDNAKNTLYLQMNSLKPEDTAVYYCAA
DPFNQGYWGQGTQVTVSS,
(SEQ ID NO: 101)
EVQVVESGGGLVQAGGSLRLSCAASGRTFTTYRMGWFRQAPGKEREFVA
AIRWSGGRTLYADSVKGRFTISRDNAKNTAYLQMNNLRPEDTAVYYCAA
DLAEYSGTYSSPADSPAGYDYWGQGTQVTVSS,
or
(SEQ ID NO: 102)
QVQLVETGGGLVQAGDSLRLSCAASGRTLSFNTYAMGWFRQAPGKEREF
VASITWNGGSTSYADSVKGRFTITRDNAKNTATLRMNSLQPDDTAVYYC
AAARYYVSGTYFPANYWGQGTQVTVSS.

In some embodiments, a method described above comprises a VHH domain that comprises a framework comprising sequence having at least 75%, 80%, 85%, 90%, 95%, 96%, 97%, 98%, 99%, or 100% sequence identity with the sequence of

(SEQ ID NO: 93)
QVQLVESGGGLVQAGGSLKLACAAPGLTFSSYRMGWFRQAPGQEREFVA
AIDWNGRGTYYRYYADSVKGRSTISRDNAKNTMYLQMNSLKPEDTAVYY
CAAGSIDLNWYGGMDYWGQGTQVTVSS,
(SEQ ID NO: 94)
EVQVVESGGGLVQAGGSLKLACAAPGLTFSSYRMGWFRQAPGQEREFVA
AIDWNGRGTYYRYYADSVKGRSTISRDNAKNTVYLQMNSLKPEDTAVYY
CAATTVLTDPRVLNEYATWGQGTQVTVSS,
(SEQ ID NO: 95)
EVQVVESGGGLVQAGGSLRLSCAVSGTSVSSNAMGWYRQAPGKQREWVG
FIDRIATTTIATSVKGRFAITRDNAKNTVYLQMSGLKPEDTAVYYCNHP
LTARWGQGTQVTVSS,
(SEQ ID NO: 96)
QVQLVESGGGLVQAGGSLRLSCAASGRTFSSYAMGWFRQAPGKEREFVA
AITWNGGTTYYADSVKGRFTISRDNAKNTVYLQMNSLKPEDTAVYYCAA
DPFNQGYWGQGTQVTVSS,
(SEQ ID NO: 97)
EVQLVESGGGLVQAGGSLRLSCAVSGSSVSSDAMGWYRQAPGNQRAWVA
FISGGGTTTYADSVKGRFTISRDNTKNTVYLHMNSLKPEDTAVYYCNHP
LTSRWGQGTQVTVSS,
(SEQ ID NO: 98)
EVQVVESGGGLVQAGGSLRLACVASRSIGSINVMGWYRQAPGKQRDLVA
RITGGGSTHYAESVKGRFTISRDNAKNTVYLQMNSLEPEDTAVYYCASM
VNPIITAWGTIGVREIPDYDYWGQGTQVTVSS,
(SEQ ID NO: 99)
QVQLVESGGGLVQAGGSLRLSCAVSGRTFSTYRMGWFRQAPGKERSFVA
AISWSGGSTTYADPVKGRFTISRDNAKNTVYLRMNSLKPEDTAVYYCND
QRGYWGQGTLVTVSS,
(SEQ ID NO: 100)
EVQVVESGGGLVQAGGSLRLSCAASGFTFTRYAMGWFRQAPGKERSFVA
AISWSGSSAGYGDSVKGRFTISRDNAKNTLYLQMNSLKPEDTAVYYCAA
DPFNQGYWGQGTQVTVSS,
(SEQ ID NO: 101)
EVQVVESGGGLVQAGGSLRLSCAASGRTFTTYRMGWFRQAPGKEREFVA
AIRWSGGRTLYADSVKGRFTISRDNAKNTAYLQMNNLRPEDTAVYYCAA
DLAEYSGTYSSPADSPAGYDYWGQGTQVTVSS,
or
(SEQ ID NO: 102)
QVQLVETGGGLVQAGDSLRLSCAASGRTLSFNTYAMGWFRQAPGKEREF
VASITWNGGSTSYADSVKGRFTITRDNAKNTATLRMNSLQPDDTAVYYC
AAARYYVSGTYFPANYWGQGTQVTVSS.

In some embodiments, a method described above comprises a VHH domain that is comprised of a sequence having at least 75%, 80%, 85%, 90%, 95%, 96%, 97%, 98%, 99%, or 100% sequence identity with the sequence of

(SEQ ID NO: 93)
QVQLVESGGGLVQAGGSLKLACAAPGLTFSSYRMGWFRQAPGQEREFVA
AIDWNGRGTYYRYYADSVKGRSTISRDNAKNTMYLQMNSLKPEDTAVYY
CAAGSIDLNWYGGMDYWGQGTQVTVSS,
(SEQ ID NO: 94)
EVQVVESGGGLVQAGGSLKLACAAPGLTFSSYRMGWFRQAPGQEREFVA
AIDWNGRGTYYRYYADSVKGRSTISRDNAKNTVYLQMNSLKPEDTAVYY
CAATTVLTDPRVLNEYATWGQGTQVTVSS,
(SEQ ID NO: 95)
EVQVVESGGGLVQAGGSLRLSCAVSGTSVSSNAMGWYRQAPGKQREWVG
FIDRIATTTIATSVKGRFAITRDNAKNTVYLQMSGLKPEDTAVYYCNHP
LTARWGQGTQVTVSS,
(SEQ ID NO: 96)
QVQLVESGGGLVQAGGSLRLSCAASGRTFSSYAMGWFRQAPGKEREFVA
AITWNGGTTYYADSVKGRFTISRDNAKNTVYLQMNSLKPEDTAVYYCAA
DPFNQGYWGQGTQVTVSS,
(SEQ ID NO: 97)
EVQLVESGGGLVQAGGSLRLSCAVSGSSVSSDAMGWYRQAPGNQRAWVA
FISGGGTTTYADSVKGRFTISRDNTKNTVYLHMNSLKPEDTAVYYCNHP
LTSRWGQGTQVTVSS,
(SEQ ID NO: 98)
EVQVVESGGGLVQAGGSLRLACVASRSIGSINVMGWYRQAPGKQRDLVA
RITGGGSTHYAESVKGRFTISRDNAKNTVYLQMNSLEPEDTAVYYCASM
VNPIITAWGTIGVREIPDYDYWGQGTQVTVSS,
(SEQ ID NO: 99)
QVQLVESGGGLVQAGGSLRLSCAVSGRTFSTYRMGWFRQAPGKERSFVA
AISWSGGSTTYADPVKGRFTISRDNAKNTVYLRMNSLKPEDTAVYYCND
QRGYWGQGTLVTVSS,
(SEQ ID NO: 100)
EVQVVESGGGLVQAGGSLRLSCAASGFTFTRYAMGWFRQAPGKERSFVA
AISWSGSSAGYGDSVKGRFTISRDNAKNTLYLQMNSLKPEDTAVYYCAA
DPFNQGYWGQGTQVTVSS,
(SEQ ID NO: 101)
EVQVVESGGGLVQAGGSLRLSCAASGRTFTTYRMGWFRQAPGKEREFVA
AIRWSGGRTLYADSVKGRFTISRDNAKNTAYLQMNNLRPEDTAVYYCAA
DLAEYSGTYSSPADSPAGYDYWGQGTQVTVSS,
or
(SEQ ID NO: 102)
QVQLVETGGGLVQAGDSLRLSCAASGRTLSFNTYAMGWFRQAPGKEREF
VASITWNGGSTSYADSVKGRFTITRDNAKNTATLRMNSLQPDDTAVYYC
AAARYYVSGTYFPANYWGQGTQVTVSS.

In some embodiments, a method described above comprises a VHH domain that comprises a CDR1 sequence, a CDR2 sequence, and a CDR3 sequence present in VHH1. Accordingly, in some embodiments, the VHH domain comprises: i) the CDR1 sequence of SYRMG (SEQ ID NO: 1), the CDR2 sequence of AIDWNGRGTYYRYYADSVKG (SEQ ID NO: 30), and the CDR3 sequence of GSIDLNWYGGMDY (SEQ ID NO: 60); ii) the CDR1 sequence of GLTFSSY (SEQ ID NO: 10), the CDR2 sequence of DWNGRGTYY (SEQ ID NO: 40) or WNGRGTY (SEQ ID NO: 260), and the CDR3 sequence of GSIDLNWYGGMDY (SEQ ID NO: 71) or SIDLNWYGGMD (SEQ ID NO: 272); iii) the CDR1 sequence of GLTFSSYR (SEQ ID NO: 20), the CDR2 sequence of IDWNGRGTYY (SEQ ID NO: 50) or IDWNGRGTYYR (SEQ ID NO: 270), and the CDR3 sequence of CAAGSIDLNWYGGMDY (SEQ ID NO: 82) or AAGSIDLNWYGGMDY (SEQ ID NO: 283); iv) the CDR1 sequence of GLTFSSYRMG (SEQ ID NO: 154), the CDR 2 sequence of AIDWNGRGTYYRYYADSVKG (SEQ ID NO: 184), and the CDR3 sequence of GSIDLNWYGGMDY (SEQ ID NO: 214); v) the CDR1 sequence of SSYRMG (SEQ ID NO: 164), the CDR2 sequence of FVAAIDWNGRGTYYRY (SEQ ID NO: 194), and the CDR3 sequence of AAGSIDLNWYGGMD (SEQ ID NO: 225); or vi) the CDR1 sequence of GLTFSSYRMG (SEQ ID NO: 174), the CDR2 sequence of AIDWNGRGTYYRY (SEQ ID NO: 204), and the CDR3 sequence of GSIDLNWYGGMDY (SEQ ID NO: 236).

In some embodiments, a method described above comprises a VHH domain that comprises a CDR1 sequence, a CDR2 sequence, and a CDR3 sequence present in VHH2. Accordingly, in some embodiments, the VHH domain comprises: i) the CDR1 sequence of SYRMG (SEQ ID NO: 1), the CDR2 sequence of AIDWNGRGTYYRYYADSVKG (SEQ ID NO: 30), and the CDR3 sequence of TTVLTDPRVLNEYAT (SEQ ID NO: 61); ii) the CDR1 sequence of GLTFSSY (SEQ ID NO: 10), the CDR2 sequence of DWNGRGTYY (SEQ ID NO: 40) or WNGRGTY (SEQ ID NO: 260), and the CDR3 sequence of TTVLTDPRVLNEYAT (SEQ ID NO: 72) or TVLTDPRVLNEYA (SEQ ID NO: 273); iii) the CDR1 sequence of GLTFSSYR (SEQ ID NO: 20), the CDR2 sequence of IDWNGRGTYY (SEQ ID NO: 50) or IDWNGRGTYYR (SEQ ID NO: 270), and the CDR3 sequence of CAATTVLTDPRVLNEYAT (SEQ ID NO: 83) or AATTVLTDPRVLNEYAT (SEQ ID NO: 284); iv) the CDR1 sequence of GLTFSSYRMG (SEQ ID NO: 154), the CDR2 sequence of AIDWNGRGTYYRYYADSVKG (SEQ ID NO: 184), and the CDR3 sequence of TTVLTDPRVLNEYAT (SEQ ID NO: 215); v) the CDR1 sequence of SSYRMG (SEQ ID NO: 164), the CDR2 sequence of FVAAIDWNGRGTYYRY (SEQ ID NO: 194), and the CDR3 sequence of AATTVLTDPRVLNEYA (SEQ ID NO: 226); or vi) the CDR1 sequence of GLTFSSYRMG (SEQ ID NO: 174), the CDR2 sequence of AIDWNGRGTYYRY (SEQ ID NO: 204), and the CDR3 sequence of TTVLTDPRVLNEYAT (SEQ ID NO: 237).

In some embodiments, a method described above comprises a VHH domain that comprises a CDR1 sequence, a CDR2 sequence, and a CDR3 sequence present in VHH3. Accordingly, in some embodiments, the VHH domain comprises: i) the CDR1 sequence of INVMG (SEQ ID NO: 2), the CDR2 sequence of RINGGGITHYAESVKG (SEQ ID NO: 31), and the CDR3 sequence of DVFGSSGYVETY (SEQ ID NO: 62); ii) the CDR1 sequence of GSIFSIN (SEQ ID NO: 11), the CDR2 sequence of NGGGI (SEQ ID NO: 41) or GGG (SEQ ID NO: 261), and the CDR3 sequence of DVFGSSGYVETY (SEQ ID NO: 73) or VFGSSGYVET (SEQ ID NO: 274); iii) the CDR1 sequence of GSIFSINV (SEQ ID NO: 21), the CDR2 sequence of INGGGIT (SEQ ID NO: 51), and the CDR3 sequence of KADVFGSSGYVETY (SEQ ID NO: 84); iv) the CDR1 sequence of GSIFSINVMG (SEQ ID NO: 155), the CDR2 sequence of RINGGGITHYAESVKG (SEQ ID NO: 185), and the CDR3 sequence of DVFGSSGYVETY (SEQ ID NO: 216); v) the CDR1 sequence of SINVMG (SEQ ID NO: 165), the CDR2 sequence of LVARINGGGITH (SEQ ID NO: 195), and the CDR3 sequence of KADVFGSSGYVET (SEQ ID NO: 227); or vi) the CDR1 sequence of GSIFSINVMG (SEQ ID NO: 175), the CDR2 sequence of RINGGGITH (SEQ ID NO: 205), and the CDR3 sequence of DVFGSSGYVETY (SEQ ID NO: 238).

In some embodiments, a method described above comprises a VHH domain that comprises a CDR1 sequence, a CDR2 sequence, and a CDR3 sequence present in VHH4. Accordingly, in some embodiments, the VHH domain comprises: i) the CDR1 sequence of SNAMG (SEQ ID NO: 3), the CDR2 sequence of FIDRIATTTIATSVKG (SEQ ID NO: 32), and the CDR3 sequence of PLTAR (SEQ ID NO: 63); ii) the CDR1 sequence of GTSVSSN (SEQ ID NO: 12), the CDR2 sequence of DRIAT (SEQ ID NO: 42) or RIA (SEQ ID NO: 262), and the CDR3 sequence of PLTAR (SEQ ID NO: 74) or LTA (SEQ ID NO: 275); iii) the CDR1 sequence of GTSVSSNA (SEQ ID NO: 22), the CDR2 sequence of IDRIATT (SEQ ID NO: 52), and the CDR3 sequence of NHPLTAR (SEQ ID NO: 85); iv) the CDR1 sequence of GTSVSSNAMG (SEQ ID NO: 156), the CDR2 sequence of FIDRIATTTIATSVKG (SEQ ID NO: 186), and the CDR3 sequence of PLTAR (SEQ ID NO: 217); v) the CDR1 sequence of SSNAMG (SEQ ID NO: 166), the CDR2 sequence of WVGFIDRIATTT (SEQ ID NO: 196), and the CDR3 sequence of NHPLTA (SEQ ID NO: 228); or vi) the CDR1 sequence of GTSVSSNAMG (SEQ ID NO: 176), the CDR2 sequence of FIDRIATTT (SEQ ID NO: 206), and the CDR3 sequence of PLTAR (SEQ ID NO: 239).

In some embodiments, a method described above comprises a VHH domain that comprises a CDR1 sequence, a CDR2 sequence, and a CDR3 sequence present in VHH5. Accordingly, in some embodiments, the VHH domain comprises: i) the CDR1 sequence of SYAMG (SEQ ID NO: 4), the CDR2 sequence of AITWNGGTTYYADSVKG (SEQ ID NO: 33), and the CDR3 sequence of DPFNQGY (SEQ ID NO: 64); ii) the CDR1 sequence of GRTFSSY (SEQ ID NO: 13), the CDR2 sequence of TWNGGT (SEQ ID NO: 43) or WNGG (SEQ ID NO: 263), and the CDR3 sequence of DPFNQGY (SEQ ID NO: 75) or PFNQG (SEQ ID NO: 276); iii) the CDR1 sequence of GRTFSSYA (SEQ ID NO: 23), the CDR2 sequence of ITWNGGTT (SEQ ID NO: 53), and the CDR3 sequence of AADPFNQGY (SEQ ID NO: 86); iv) the CDR1 sequence of GRTFSSYAMG (SEQ ID NO: 157), the CDR2 sequence of AITWNGGTTYYADSVKG (SEQ ID NO: 187), and the CDR3 sequence of DPFNQGY (SEQ ID NO: 218); v) the CDR1 sequence of SSYAMG (SEQ ID NO: 167), the CDR2 sequence of FVAAITWNGGTTY (SEQ ID NO: 197), and the CDR3 sequence of AADPFNQG (SEQ ID NO: 229); or vi) the CDR1 sequence of GRTFSSYAMG (SEQ ID NO: 177), the CDR2 sequence of AITWNGGTTY (SEQ ID NO: 207), and the CDR3 sequence of DPFNQGY (SEQ ID NO: 240).

In some embodiments, a method described above comprises a VHH domain that comprises a CDR1 sequence, a CDR2 sequence, and a CDR3 sequence present in VHH6. Accordingly, in some embodiments, the VHH domain comprises: i) the CDR1 sequence of SDAMG (SEQ ID NO: 5), the CDR2 sequence of FISGGGTTTYADSVKG (SEQ ID NO: 34), and the CDR3 sequence of PLTSR (SEQ ID NO: 65); ii) the CDR1 sequence of GSSVSSD (SEQ ID NO: 14), the CDR2 sequence of SGGGT (SEQ ID NO: 44) or GGG (SEQ ID NO: 264), and the CDR3 sequence of PLTSR (SEQ ID NO: 76) or LTS (SEQ ID NO: 277); iii) the CDR1 sequence of GSSVSSDA (SEQ ID NO: 24), the CDR2 sequence of ISGGGTT (SEQ ID NO: 54), and the CDR3 sequence of NHPLTSR (SEQ ID NO: 87); iv) the CDR1 sequence of GSSVSSDAMG (SEQ ID NO: 158), the CDR2 sequence of FISGGGTTTYADSVKG (SEQ ID NO: 188), and the CDR3 sequence of PLTSR (SEQ ID NO: 219); v) the CDR1 sequence of SSDAMG (SEQ ID NO: 168), the CDR2 sequence of WVAFISGGGTTT (SEQ ID NO: 198), and the CDR3 sequence of NHPLTS (SEQ ID NO: 230); or vi) the CDR1 sequence of GSSVSSDAMG (SEQ ID NO: 178), the CDR2 sequence of FISGGGTTT (SEQ ID NO: 208), and the CDR3 sequence of PLTSR (SEQ ID NO: 241).

In some embodiments, a method described above comprises a VHH domain that comprises a CDR1 sequence, a CDR2 sequence, and a CDR3 sequence present in VHH7. Accordingly, in some embodiments, the VHH domain comprises: i) the CDR1 sequence of INVMG (SEQ ID NO: 6), the CDR2 sequence of RITGGGSTHYAESVKG (SEQ ID NO: 35), and the CDR3 sequence of MVNPIITAWGTIGVREIPDYDY (SEQ ID NO: 66); ii) the CDR1 sequence of RSIGSIN (SEQ ID NO: 15), the CDR2 sequence of TGGGS (SEQ ID NO: 45) or GGG (SEQ ID NO: 265), and the CDR3 sequence of MVNPIITAWGTIGVREIPDYDY (SEQ ID NO: 77) or VNPIITAWGTIGVREIPDYD (SEQ ID NO: 278); iii) the CDR1 sequence of RSIGSINV (SEQ ID NO: 25), the CDR2 sequence of ITGGGST (SEQ ID NO: 55), and the CDR3 sequence of ASMVNPIITAWGTIGVREIPDYDY (SEQ ID NO: 88); iv) the CDR1 sequence of RSIGSINVMG (SEQ ID NO: 159), the CDR2 sequence of RITGGGSTHYAESVKG (SEQ ID NO: 189), and the CDR3 sequence of MVNPIITAWGTIGVREIPDYDY (SEQ ID NO: 220); v) the CDR1 sequence of SINVMG (SEQ ID NO: 169), the CDR2 sequence of LVARITGGGSTH (SEQ ID NO: 199), and the CDR3 sequence of ASMVNPIITAWGTIGVREIPDYD (SEQ ID NO: 231); or vi) the CDR1 sequence of RSIGSINVMG (SEQ ID NO: 179), the CDR2 sequence of RITGGGSTH (SEQ ID NO: 209), and the CDR3 sequence of MVNPIITAWGTIGVREIPDYDY (SEQ ID NO: 242).

In some embodiments, a method described above comprises a VHH domain that comprises a CDR1 sequence, a CDR2 sequence, and a CDR3 sequence present in VHH9. Accordingly, in some embodiments, the VHH domain comprises: i) the CDR1 sequence of TYRMG (SEQ ID NO: 7), the CDR2 sequence of AISWSGGSTTYADPVKG (SEQ ID NO: 36), and the CDR3 sequence of DQRGY (SEQ ID NO: 67) or QRGY (SEQ ID NO: 271); ii) the CDR1 sequence of GRTFSTY (SEQ ID NO: 16), the CDR2 sequence of SWSGGS (SEQ ID NO: 46) or WSGG (SEQ ID NO: 266), and the CDR3 sequence of DQRGY (SEQ ID NO: 78) or RG (SEQ ID NO: 279); iii) the CDR1 sequence of GRTFSTYR (SEQ ID NO: 26), the CDR2 sequence of ISWSGGST (SEQ ID NO: 56), and the CDR3 sequence of NDQRGY (SEQ ID NO: 89); iv) the CDR1 sequence of GRTFSTYRMG (SEQ ID NO: 160), the CDR2 sequence of AISWSGGSTTYADPVKG (SEQ ID NO: 190), and the CDR3 sequence of QRGY (SEQ ID NO: 221); v) the CDR1 sequence of STYRMG (SEQ ID NO: 170), the CDR2 sequence of FVAAISWSGGSTT (SEQ ID NO: 200), and the CDR3 sequence of NDQRG (SEQ ID NO: 232); or vi) the CDR1 sequence of GRTFSTYRMG (SEQ ID NO: 180), the CDR2 sequence of AISWSGGSTT (SEQ ID NO: 210), and the CDR3 sequence of QRGY (SEQ ID NO: 243).

In some embodiments, a method described above comprises a VHH domain that comprises a CDR1 sequence, a CDR2 sequence, and a CDR3 sequence present in VHH10. Accordingly, in some embodiments, the VHH domain comprises: i) the CDR1 sequence of RYAMG (SEQ ID NO: 8), the CDR2 sequence of AISWSGSSAGYGDSVKG (SEQ ID NO: 37), and the CDR3 sequence of DPFNQGY (SEQ ID NO: 68); ii) the CDR1 sequence of GFTFTRY (SEQ ID NO: 17), the CDR2 sequence of SWSGSS (SEQ ID NO: 47) or WSGS (SEQ ID NO: 267), and the CDR3 sequence of DPFNQGY (SEQ ID NO: 79) or PFNQG (SEQ ID NO: 280); iii) the CDR1 sequence of GFTFTRYA (SEQ ID NO: 27), the CDR2 sequence of ISWSGSSA (SEQ ID NO: 57), and the CDR3 sequence of AADPFNQGY (SEQ ID NO: 90); iv) the CDR1 sequence of GFTFTRYAMG (SEQ ID NO: 161), the CDR2 sequence of AISWSGSSAGYGDSVKG (SEQ ID NO: 191), and the CDR3 sequence of DPFNQGY (SEQ ID NO: 222); v) the CDR1 sequence of TRYAMG (SEQ ID NO: 171), the CDR2 sequence of FVAAISWSGSSAG (SEQ ID NO: 201), and the CDR3 sequence of AADPFNQG (SEQ ID NO: 233); or vi) the CDR1 sequence of GFTFTRYAMG (SEQ ID NO: 181), the CDR2 sequence of AISWSGSSAG (SEQ ID NO: 211), and the CDR3 sequence of DPFNQGY (SEQ ID NO: 244).

In some embodiments, a method described above comprises a VHH domain that comprises a CDR1 sequence, a CDR2 sequence, and a CDR3 sequence present in VHH11. Accordingly, in some embodiments, the VHH domain comprises: i) the CDR1 sequence of FTTYRMG (SEQ ID NO: 258) or TYRMG (SEQ ID NO: 259), the CDR2 sequence of AIRWSGGRTLYADSVKG (SEQ ID NO: 38), and the CDR3 sequence of DLAEYSGTYSSPADSPAGYDY (SEQ ID NO: 69); ii) the CDR1 sequence of GRTFTTY (SEQ ID NO: 18), the CDR2 sequence of RWSGGR (SEQ ID NO: 48) or WSGG (SEQ ID NO: 268), and the CDR3 sequence of DLAEYSGTYSSPADSPAGYDY (SEQ ID NO: 80) or LAEYSGTYSSPADSPAGYD (SEQ ID NO: 281); iii) the CDR1 sequence of GRTFTTYR (SEQ ID NO: 28), the CDR2 sequence of IRWSGGRT (SEQ ID NO: 58), and the CDR3 sequence of AADLAEYSGTYSSPADSPAGYDY (SEQ ID NO: 91); iv) the CDR1 sequence of GRTFTTYRMG (SEQ ID NO: 162), the CDR2 sequence of AIRWSGGRTLYADSVKG (SEQ ID NO: 192), and the CDR3 sequence of DLAEYSGTYSSPADSPAGYDY (SEQ ID NO: 223); v) the CDR1 sequence of TTYRMG (SEQ ID NO: 172), the CDR2 sequence of FVAAIRWSGGRTL (SEQ ID NO: 202), and the CDR3 sequence of AADLAEYSGTYSSPADSPAGYD (SEQ ID NO: 234); or vi) the CDR1 sequence of GRTFTTYRMG (SEQ ID NO: 182), the CDR2 sequence of AIRWSGGRTL (SEQ ID NO: 212), and the CDR3 sequence of DLAEYSGTYSSPADSPAGYDY (SEQ ID NO: 245).

In some embodiments, a method described above comprises a VHH domain that comprises a CDR1 sequence, a CDR2 sequence, and a CDR3 sequence present in VHH12. Accordingly, in some embodiments, the VHH domain comprises: i) the CDR1 sequence of FNTYAMG (SEQ ID NO: 9), the CDR2 sequence of SITWNGGSTSYADSVKG (SEQ ID NO: 39), and the CDR3 sequence of ARYYVSGTYFPANY (SEQ ID NO: 70); ii) the CDR1 sequence of GRTLSFNTY (SEQ ID NO: 19), the CDR2 sequence of TWNGGS (SEQ ID NO: 49) or WNGG (SEQ ID NO: 269), and the CDR3 sequence of ARYYVSGTYFPANY (SEQ ID NO: 81) or RYYVSGTYFPAN (SEQ ID NO: 282); iii) the CDR1 sequence of GRTLSFNTYA (SEQ ID NO: 29), the CDR2 sequence of ITWNGGST (SEQ ID NO: 59), and the CDR3 sequence of AAARYYVSGTYFPANY (SEQ ID NO: 92); iv) the CDR1 sequence of GRTLSFNTYAMG (SEQ ID NO: 163), the CDR2 sequence of SITWNGGSTSYADSVKG (SEQ ID NO: 193), and the CDR3 sequence of ARYYVSGTYFPANY (SEQ ID NO: 224); v) the CDR1 sequence of SFNTYAMG (SEQ ID NO: 173), the CDR2 sequence of FVASITWNGGSTS (SEQ ID NO: 203), and the CDR3 sequence of AAARYYVSGTYFPAN (SEQ ID NO: 235); or vi) the CDR1 sequence of GRTLSFNTYAMG (SEQ ID NO: 183), the CDR2 sequence of SITWNGGSTS (SEQ ID NO: 213), and the CDR3 sequence of ARYYVSGTYFPANY (SEQ ID NO: 246).

In some embodiments, a method described above comprises a therapeutic molecule that comprise VHH domain and an agent, wherein the agent is an antibody or fragment thereof, a peptide, a vaccine, a small molecule, a polynucleotide, a radioisotope, a toxin, an enzyme, an anticoagulant, a hormone, a cytokine, an anti-inflammatory molecule, an RNAi, an mRNA, a self-replicating RNA, an antibiotic, or an antibody-antibiotic conjugate. In some embodiments, the agent is an antibiotic. In some embodiments, the VHH domain is genetically fused or chemically conjugated to the agent. In some embodiments, the method further comprises a linker between the VHH domain and the agent. In some embodiments, the linker is a polypeptide. In some embodiments, the linker is a flexible linker comprising a sequence selected from the group consisting of EPKTPKPQPQPQLQPQPNPTTESKSPK (SEQ ID NO: 130), (EAAAK)n (SEQ ID NO: 147), (GGGGS)n (SEQ ID NO: 148) and (GGGS)n (SEQ ID NO: 149), wherein n is an integer from 1 to 20. In some embodiments, the VHH domain is chemically-conjugated to the agent. In some embodiments, the VHH domain is non-covalently bound to the agent. In some embodiments, the method does not inhibit pIgR-mediated transcytosis of IgA.

In some embodiments, a method described above comprises a VHH domain that comprises a CDR1 sequence of SNAMG (SEQ ID NO: 3), INVMG (SEQ ID NO: 6), TYRMG (SEQ ID NO: 7), RYAMG (SEQ ID NO: 8), FTTYRMG (SEQ ID NO: 258), TYRMG (SEQ ID NO: 259), FNTYAMG (SEQ ID NO: 9), GTSVSSN (SEQ ID NO: 12), GRTFSSY (SEQ ID NO: 13), RSIGSIN (SEQ ID NO: 15), GRTFSTY (SEQ ID NO: 16), GFTFTRY (SEQ ID NO: 17), GRTFTTY (SEQ ID NO: 18), GRTLSFNTY (SEQ ID NO: 19), GTSVSSNA (SEQ ID NO: 22), RSIGSINV (SEQ ID NO: 25), GRTFSTYR (SEQ ID NO: 26), GFTFTRYA (SEQ ID NO: 27), GRTFTTYR (SEQ ID NO: 28), GRTLSFNTYA (SEQ ID NO: 29), GTSVSSNAMG (SEQ ID NO: 156), RSIGSINVMG (SEQ ID NO: 159), GRTFSTYRMG (SEQ ID NO: 160), GFTFTRYAMG (SEQ ID NO: 161), GRTFTTYRMG (SEQ ID NO: 162), GRTLSFNTYAMG (SEQ ID NO: 163), SSNAMG (SEQ ID NO: 166), SINVMG (SEQ ID NO: 169), STYRMG (SEQ ID NO: 170), TRYAMG (SEQ ID NO: 171), TTYRMG (SEQ ID NO: 172), SFNTYAMG (SEQ ID NO: 173), GTSVSSNAMG (SEQ ID NO: 176), RSIGSINVMG (SEQ ID NO: 179), GRTFSTYRMG (SEQ ID NO: 180), GFTFTRYAMG (SEQ ID NO: 181), GRTFTTYRMG (SEQ ID NO: 182), or GRTLSFNTYAMG (SEQ ID NO: 183).

In some embodiments, a method described above comprises a VHH domain that comprises a CDR2 sequence of FIDRIATTTIATSVKG (SEQ ID NO: 32), RITGGGSTHYAESVKG (SEQ ID NO: 35), AISWSGGSTTYADPVKG (SEQ ID NO: 36), AISWSGSSAGYGDSVKG (SEQ ID NO: 37), AIRWSGGRTLYADSVKG (SEQ ID NO: 38), SITWNGGSTSYADSVKG (SEQ ID NO: 39), DRIAT (SEQ ID NO: 42), RIA (SEQ ID NO: 262), TGGGS (SEQ ID NO: 45), GGG (SEQ ID NO: 265), SWSGGS (SEQ ID NO: 46), WSGG (SEQ ID NO: 266), SWSGSS (SEQ ID NO: 47), WSGS (SEQ ID NO: 267), RWSGGR (SEQ ID NO: 48), WSGG (SEQ ID NO: 268), TWNGGS (SEQ ID NO: 49), WNGG (SEQ ID NO: 269), IDRIATT (SEQ ID NO: 52), ITGGGST (SEQ ID NO: 55), ISWSGGST (SEQ ID NO: 56), ISWSGSSA (SEQ ID NO: 57), IRWSGGRT (SEQ ID NO: 58), ITWNGGST (SEQ ID NO: 59), FIDRIATTTIATSVKG (SEQ ID NO: 186), RITGGGSTHYAESVKG (SEQ ID NO: 189), AISWSGGSTTYADPVKG (SEQ ID NO: 190), AISWSGSSAGYGDSVKG (SEQ ID NO: 191), AIRWSGGRTLYADSVKG (SEQ ID NO: 192), SITWNGGSTSYADSVKG (SEQ ID NO: 193), WVGFIDRIATTT (SEQ ID NO: 196), LVARITGGGSTH (SEQ ID NO: 199), FVAAISWSGGSTT (SEQ ID NO: 200), FVAAISWSGSSAG (SEQ ID NO: 201), FVAAIRWSGGRTL (SEQ ID NO: 202), FVASITWNGGSTS (SEQ ID NO: 203), FIDRIATTT (SEQ ID NO: 206), RITGGGSTH (SEQ ID NO: 209), AISWSGGSTT (SEQ ID NO: 210), AISWSGSSAG (SEQ ID NO: 211), AIRWSGGRTL (SEQ ID NO: 212), or SITWNGGSTS (SEQ ID NO: 213).

In some embodiments, a method described above comprises a VHH domain that comprises a CDR3 sequence of PLTAR (SEQ ID NO: 63), MVNPIITAWGTIGVREIPDYDY (SEQ ID NO: 66), DQRGY (SEQ ID NO: 67), QRGY (SEQ ID NO: 271), DPFNQGY (SEQ ID NO: 68), DLAEYSGTYSSPADSPAGYDY (SEQ ID NO: 69), ARYYVSGTYFPANY (SEQ ID NO: 70), PLTAR (SEQ ID NO: 74), LTA (SEQ ID NO: 275), MVNPIITAWGTIGVREIPDYDY (SEQ ID NO: 77), VNPIITAWGTIGVREIPDYD (SEQ ID NO: 278), DQRGY (SEQ ID NO: 78), RG (SEQ ID NO: 279), DPFNQGY (SEQ ID NO: 79), PFNQG (SEQ ID NO: 280), DLAEYSGTYSSPADSPAGYDY (SEQ ID NO: 80), LAEYSGTYSSPADSPAGYD (SEQ ID NO: 281), ARYYVSGTYFPANY (SEQ ID NO: 81), RYYVSGTYFPAN (SEQ ID NO: 282), NHPLTAR (SEQ ID NO: 85), ASMVNPIITAWGTIGVREIPDYDY (SEQ ID NO: 88), NDQRGY (SEQ ID NO: 89), AADPFNQGY (SEQ ID NO: 90), AADLAEYSGTYSSPADSPAGYDY (SEQ ID NO: 91), AAARYYVSGTYFPANY (SEQ ID NO: 92), PLTAR (SEQ ID NO: 217), MVNPIITAWGTIGVREIPDYDY (SEQ ID NO: 220), QRGY (SEQ ID NO: 221), DPFNQGY (SEQ ID NO: 222), DLAEYSGTYSSPADSPAGYDY (SEQ ID NO: 223), ARYYVSGTYFPANY (SEQ ID NO: 224), NHPLTA (SEQ ID NO: 228), ASMVNPIITAWGTIGVREIPDYD (SEQ ID NO: 231), NDQRG (SEQ ID NO: 232), AADPFNQG (SEQ ID NO: 233), AADLAEYSGTYSSPADSPAGYD (SEQ ID NO: 234), AAARYYVSGTYFPAN (SEQ ID NO: 235), PLTAR (SEQ ID NO: 239), MVNPIITAWGTIGVREIPDYDY (SEQ ID NO: 242), QRGY (SEQ ID NO: 243), DPFNQGY (SEQ ID NO: 244), DLAEYSGTYSSPADSPAGYDY (SEQ ID NO: 245), or ARYYVSGTYFPANY (SEQ ID NO: 246).

In another aspect, provided herein is a method for delivering from an apical surface of a polymeric immunoglobulin receptor (pIgR)-expressing cell to a basolateral surface of the pIgR-expressing cell comprising contacting the pIgR-expressing cell with (i) a single domain antibody that binds to pIgR, or (ii) a therapeutic molecule comprising an agent and the single domain antibody.

In another aspect, provided herein is a method for transporting a therapeutic molecule to a basolateral surface of the pIgR-expressing cell of a subject, comprising administering to the subject the therapeutic molecule comprising an agent and a single domain antibody. In some embodiments, the therapeutic molecule is administered to the subject via oral delivery, buccal delivery, nasal delivery or inhalation delivery. In some embodiments, the therapeutic agent is transported from an apical surface of a pIgR-expressing cell to a basolateral surface of the pIgR-expressing cell in the subject.

In another aspect, provided herein is a method for transporting a therapeutic molecule to systemic circulation of a subject, comprising administering to the subject the therapeutic molecule comprising an agent and a single domain antibody, wherein the therapeutic molecule is administered to the subject via oral delivery, buccal delivery, nasal delivery or inhalation delivery. In some embodiments, the therapeutic agent is transported from an apical surface of a pIgR-expressing cell to a basolateral surface of the pIgR-expressing cell in the subject.

In yet another aspect, provided herein is a method for transporting a therapeutic molecule to Lamina propria or gastrointestinal tract of a subject, comprising administering to the subject the therapeutic molecule comprising an agent and a single domain antibody, wherein the therapeutic molecule is administered to the subject via oral delivery, buccal delivery, nasal delivery or inhalation delivery. In some embodiments, the therapeutic agent is transported from an apical surface of a pIgR-expressing cell to a basolateral surface of the pIgR-expressing cell in the subject.

In some embodiments, the single domain antibody or the therapeutic molecule comprising the agent and the single domain antibody is capable of being transported from the basolateral surface of the pIgR-expressing cell to the apical surface of the pIgR-expressing cell.

In some embodiments, the pIgR-expressing cell is an epithelial cell. In some embodiments, the epithelia cell is an intestinal lumen cell or an airway epithelial cell.

In some embodiments, the agent is a diabetes medication. In some embodiments, the diabetes medication is selected from a group consisting of insulin, glucagon-like-peptide-1, insulin-mimic peptides, and glucagon-like-peptide-1-mimic peptides.

In some embodiments, the agent is a peptide or an antibody or a fragment thereof. In some embodiments, the antibody or fragment thereof is selected from a group consisting of an anti-TNF-alpha antibody or a fragment thereof, an anti-IL23 antibody or a fragment thereof, and an antibody that binds to a receptor of IL23 or a fragment thereof.

In some embodiments, the agent is a vaccine. In some embodiments, the vaccine is for preventing an infection selected from a group consisting of Vibrio, Cholera, Typhoid, Rotavirus, Tuberculosis, HIV, Flu, Ebola, and Sendai.

In another aspect, provide herein is a process for providing a molecule to a subject, comprising administering to the subject the molecule comprising an agent and a single domain antibody that binds to polymeric immunoglobulin receptor (pIgR), wherein the molecule is administered to the subject via oral delivery, buccal delivery, nasal delivery or inhalation delivery.

In some embodiments, the molecule is capable of being provided to a basolateral surface of an pIgR-expressing cell from an apical surface of the pIgR-expressing cell in the subject.

In some embodiments, the molecule is capable of being provided to an apical surface of the pIgR-expressing cell from a basolateral surface of an pIgR-expressing cell in the subject.

In some embodiments, the pIgR-expressing cell is an epithelial cell. In some embodiments, the epithelia cell is an intestinal lumen cell or an airway epithelial cell.

In some embodiments, the agent is a diabetes medication. In some embodiments, the diabetes medication is selected from a group consisting of insulin, glucagon-like-peptide-1, insulin-mimic peptides, and glucagon-like-peptide-1-mimic peptides.

In some embodiments, the agent is a peptide or an antibody or a fragment thereof. In some embodiments, the antibody or fragment thereof is selected from a group consisting of an anti-TNF-alpha antibody or a fragment thereof, an anti-IL23 antibody or a fragment thereof, and an antibody that binds to a receptor of IL23 or a fragment thereof.

In some embodiments, the agent is a vaccine. In some embodiments, the vaccine is for preventing an infection selected from a group consisting of Vibrio, Cholera, Typhoid, Rotavirus, Tuberculosis, HIV, Flu, Ebola, and Sendai.

In another aspect, provided herein is a process comprising steps for providing a molecule to a subject.

In some embodiments, the molecule comprises an agent and a single domain antibody that binds to pIgR.

In some embodiments, the agent is an antibody or fragment thereof, a peptide, a vaccine, a small molecule, a polynucleotide, a radioisotope, a toxin, an enzyme, an anticoagulant, a hormone, a cytokine, an anti-inflammatory molecule, an RNAi, an antibiotic, or an antibody-antibiotic conjugate.

In some embodiments, the agent is an antibody or fragment thereof, a peptide, or a vaccine.

In some embodiments, the single domain antibody is genetically fused or chemically conjugated to the agent.

In one aspect, provided herein is a system for providing a molecule to Lamina propria or gastrointestinal tract of a subject, comprising a molecule suitable for administering to the subject, the molecule comprising an agent and a single domain antibody that binds to pIgR, wherein the molecule is administered to the subject via oral delivery, buccal delivery, nasal delivery or inhalation delivery, or a combination thereof.

In some embodiments, the agent is a diabetes medication. In some embodiments, the diabetes medication is selected from a group consisting of insulin, glucagon-like-peptide-1, insulin-mimic peptides, and glucagon-like-peptide-1-mimic peptides.

In some embodiments, the agent is a peptide or an antibody or a fragment thereof. In some embodiments, the antibody or fragment thereof is selected from a group consisting of an anti-TNF-alpha antibody or a fragment thereof, an anti-IL23 antibody or a fragment thereof, and an antibody that binds to a receptor of IL23 or a fragment thereof.

In some embodiments, the agent is a vaccine. In some embodiments, the vaccine is for preventing an infection selected from a group consisting of Vibrio, Cholera, Typhoid, Rotavirus, Tuberculosis, HIV, Flu, Ebola, and Sendai.

In another aspect, provided herein is a system comprising a means for providing a molecule to Lamina propria or gastrointestinal tract of a subject.

In some embodiments, the molecule comprises an agent and a single domain antibody that binds to pIgR.

In some embodiments, the agent is an antibody or fragment thereof, a peptide, a vaccine, a small molecule, a polynucleotide, a radioisotope, a toxin, an enzyme, an anticoagulant, a hormone, a cytokine, an anti-inflammatory molecule, an RNAi, an antibiotic, or an antibody-antibiotic conjugate.

In some embodiments, the agent is an antibody or fragment thereof, a peptide, or a vaccine.

In some embodiments, the single domain antibody is genetically fused or chemically conjugated to the agent.

In some embodiments, the single domain antibody binds to an extracellular domain 1, an extracellular domain 2, an extracellular domain 1-2, an extracellular domain 3, an extracellular domain 2-3, an extracellular domain 4-5, or an extracellular domain 5 of pIgR.

In some embodiments, the single domain antibody binds to an extracellular domain 1 of pIgR. In some embodiments, the single domain antibody binds to an extracellular domain 2 of pIgR. In some embodiments, the single domain antibody binds to an extracellular domain 1-2 of pIgR. In some embodiments, the single domain antibody binds to an extracellular domain 3 of pIgR. In some embodiments, the single domain antibody binds to an extracellular domain 2-3 of pIgR. In some embodiments, the single domain antibody binds to an extracellular domain 4-5 of pIgR. In some embodiments, the single domain antibody binds to an extracellular domain 5 of pIgR.

In some embodiments, the single domain antibody competes with IgA binding to the pIgR. In some embodiments, the single domain antibody promotes IgA binding to the pIgR.

In some embodiments, the K_{D} of the binding of the single domain antibody to pIgR is from about 4 to about 525 nM. In some embodiments, the K_{D} of the binding of the single domain antibody to pIgR is less than about 50 nM. In some embodiments, the K_{D} of the binding of the single domain antibody to pIgR is from about 4 to about 34 nM.

In some embodiments, the Tₘ of the single domain antibody is from about 53 to about 77° C. In other embodiments, the Tₘ of the single domain antibody is from 53.9 to 76.4° C.

In some embodiments, pIgR is human pIgR. In other embodiments, pIgR is mouse pIgR.

In some embodiments, the single domain antibody provided herein does not bind to a stalk sequence of human pIgR (e.g., SEQ ID NO:143 and/or a stalk sequence of mouse pIgR (e.g., SEQ ID NO:144 or SEQ ID NO:145).

In some embodiments, the single domain antibody comprises a CDR3 sequence of

(SEQ ID NO: 60)
GSIDLNWYGGMDY,
(SEQ ID NO: 61)
TTVLTDPRVLNEYAT,
(SEQ ID NO: 62)
DVFGSSGYVETY,
(SEQ ID NO: 63)
PLTAR,
(SEQ ID NO: 64)
DPFNQGY,
(SEQ ID NO: 65)
PLTSR,
(SEQ ID NO: 66)
MVNPIITAWGTIGVREIPDYDY,
(SEQ ID NO: 67)
DQRGY,
(SEQ ID NO: 271)
QRGY,
(SEQ ID NO: 68)
DPFNQGY,
(SEQ ID NO: 69)
DLAEYSGTYSSPADSPAGYDY,
(SEQ ID NO: 70)
ARYYVSGTYFPANY,
(SEQ ID NO: 71)
GSIDLNWYGGMDY,
(SEQ ID NO: 272)
SIDLNWYGGMD,
(SEQ ID NO: 72)
TTVLTDPRVLNEYAT,
(SEQ ID NO: 273)
TVLTDPRVLNEYA,
(SEQ ID NO: 73)
DVFGSSGYVETY,
(SEQ ID NO: 274)
VFGSSGYVET,
(SEQ ID NO: 74)
PLTAR,
(SEQ ID NO: 275)
LTA,
(SEQ ID NO: 75)
DPFNQGY,
(SEQ ID NO: 276)
PFNQG,
(SEQ ID NO: 76)
PLTSR,
(SEQ ID NO: 277)
LTS,
(SEQ ID NO: 77)
MVNPIITAWGTIGVREIPDYDY,
(SEQ ID NO: 278)
VNPIITAWGTIGVREIPDYD,
(SEQ ID NO: 78)
DQRGY,
(SEQ ID NO: 279)
RG,
(SEQ ID NO: 79)
DPFNQGY,
(SEQ ID NO: 280)
PFNQG,
(SEQ ID NO: 80)
DLAEYSGTYSSPADSPAGYDY,
(SEQ ID NO: 281)
LAEYSGTYSSPADSPAGYD,
(SEQ ID NO: 81)
ARYYVSGTYFPANY,
(SEQ ID NO: 282)
RYYVSGTYFPAN,
(SEQ ID NO: 82)
CAAGSIDLNWYGGMDY,
(SEQ ID NO: 283)
AAGSIDLNWYGGMDY,
(SEQ ID NO: 83)
CAATTVLTDPRVLNEYAT,
(SEQ ID NO: 284)
AATTVLTDPRVLNEYAT,
(SEQ ID NO: 84)
KADVFGSSGYVETY,
(SEQ ID NO: 85)
NHPLTAR,
(SEQ ID NO: 86)
AADPFNQGY,
(SEQ ID NO: 87)
NHPLTSR,
(SEQ ID NO: 88)
ASMVNPIITAWGTIGVREIPDYDY,
(SEQ ID NO: 89)
NDQRGY,
(SEQ ID NO: 90)
AADPFNQGY,
(SEQ ID NO: 91)
AADLAEYSGTYSSPADSPAGYDY,
(SEQ ID NO: 92)
AAARYYVSGTYFPANY,
(SEQ ID NO: 214)
GSIDLNWYGGMDY,
(SEQ ID NO: 215)
TTVLTDPRVLNEYAT,
(SEQ ID NO: 216)
DVFGSSGYVETY,
(SEQ ID NO: 217)
PLTAR,
(SEQ ID NO: 218)
DPFNQGY,
(SEQ ID NO: 219)
PLTSR,
(SEQ ID NO: 220)
MVNPIITAWGTIGVREIPDYDY,
(SEQ ID NO: 221)
QRGY,
(SEQ ID NO: 222)
DPFNQGY,
(SEQ ID NO: 223)
DLAEYSGTYSSPADSPAGYDY,
(SEQ ID NO: 224)
ARYYVSGTYFPANY,
(SEQ ID NO: 225)
AAGSIDLNWYGGMD,
(SEQ ID NO: 226)
AATTVLTDPRVLNEYA,
(SEQ ID NO: 227)
KADVFGSSGYVET,
(SEQ ID NO: 228)
NHPLTA,
(SEQ ID NO: 229)
AADPFNQG,
(SEQ ID NO: 230)
NHPLTS,
(SEQ ID NO: 231)
ASMVNPIITAWGTIGVREIPDYD,
(SEQ ID NO: 232)
NDQRG,
(SEQ ID NO: 233)
AADPFNQG,
(SEQ ID NO: 234)
AADLAEYSGTYSSPADSPAGYD,
(SEQ ID NO: 235)
AAARYYVSGTYFPAN,
(SEQ ID NO 236)
GSIDLNWYGGMDY,
(SEQ ID NO: 237)
TTVLTDPRVLNEYAT,
(SEQ ID NO: 238)
DVFGSSGYVETY,
(SEQ ID NO: 239)
PLTAR,
(SEQ ID NO: 240)
DPFNQGY,
(SEQ ID NO: 241)
PLTSR,
(SEQ ID NO: 242)
MVNPIITAWGTIGVREIPDYDY,
(SEQ ID NO: 243)
QRGY,
(SEQ ID NO: 244)
DPFNQGY,
(SEQ ID NO: 245)
DLAEYSGTYSSPADSPAGYDY,
or
(SEQ ID NO: 246)
ARYYVSGTYFPANY.

In some embodiments, the single domain antibody comprises a CDR2 sequence of

(SEQ ID NO: 30)
AIDWNGRGTYYRYYADSVKG,
(SEQ ID NO: 31)
RINGGGITHYAESVKG,
(SEQ ID NO: 32)
FIDRIATTTIATSVKG,
(SEQ ID NO: 33)
AITWNGGTTYYADSVKG,
(SEQ ID NO: 34)
FISGGGTTTYADSVKG,
(SEQ ID NO: 35)
RITGGGSTHYAESVKG,
(SEQ ID NO: 36)
AISWSGGSTTYADPVKG,
(SEQ ID NO: 37)
AISWSGSSAGYGDSVKG,
(SEQ ID NO: 38)
AIRWSGGRTLYADSVKG,
(SEQ ID NO: 39)
SITWNGGSTSYADSVKG,
(SEQ ID NO: 40)
DWNGRGTYY,
(SEQ ID NO: 260)
WNGRGTY,
(SEQ ID NO: 41)
NGGGI,
(SEQ ID NO: 261)
GGG,
(SEQ ID NO: 42)
DRIAT,
(SEQ ID NO: 262)
RIA,
(SEQ ID NO: 43)
TWNGGT,
(SEQ ID NO: 263)
WNGG,
(SEQ ID NO: 44)
SGGGT,
(SEQ ID NO: 264)
GGG,
(SEQ ID NO: 45)
TGGGS,
(SEQ ID NO: 265)
GGG,
(SEQ ID NO: 46)
SWSGGS,
(SEQ ID NO: 266)
WSGG,
(SEQ ID NO: 47)
SWSGSS,
(SEQ ID NO: 267)
WSGS,
(SEQ ID NO: 48)
RWSGGR,
(SEQ ID NO: 268)
WSGG,
(SEQ ID NO: 49)
TWNGGS,
(SEQ ID NO: 269)
WNGG,
(SEQ ID NO: 50)
IDWNGRGTYY,
(SEQ ID NO: 270)
IDWNGRGTYYR,
(SEQ ID NO: 51)
INGGGIT,
(SEQ ID NO: 52)
IDRIATT,
(SEQ ID NO: 53)
ITWNGGTT,
(SEQ ID NO: 54)
ISGGGTT,
(SEQ ID NO: 55)
ITGGGST,
(SEQ ID NO: 56)
ISWSGGST,
(SEQ ID NO: 57)
ISWSGSSA,
(SEQ ID NO: 58)
IRWSGGRT,
(SEQ ID NO: 59)
ITWNGGST,
(SEQ ID NO: 184)
AIDWNGRGTYYRYYADSVKG,
(SEQ ID NO: 185)
RINGGGITHYAESVKG,
(SEQ ID NO: 186)
FIDRIATTTIATSVKG,
(SEQ ID NO: 187)
AITWNGGTTYYADSVKG,
(SEQ ID NO: 188)
FISGGGTTTYADSVKG,
(SEQ ID NO: 189)
RITGGGSTHYAESVKG,
(SEQ ID NO: 190)
AISWSGGSTTYADPVKG,
(SEQ ID NO: 191)
AISWSGSSAGYGDSVKG,
(SEQ ID NO: 192)
AIRWSGGRTLYADSVKG,
(SEQ ID NO: 193)
SITWNGGSTSYADSVKG,
(SEQ ID NO: 194)
FVAAIDWNGRGTYYRY,
(SEQ ID NO: 195)
LVARINGGGITH,
(SEQ ID NO: 196)
WVGFIDRIATTT,
(SEQ ID NO: 197)
FVAAITWNGGTTY,
(SEQ ID NO: 198)
WVAFISGGGTTT,
(SEQ ID NO: 199)
LVARITGGGSTH,
(SEQ ID NO: 200)
FVAAISWSGGSTT,
(SEQ ID NO: 201)
FVAAISWSGSSAG,
(SEQ ID NO: 202)
FVAAIRWSGGRTL,
(SEQ ID NO: 203)
FVASITWNGGSTS,
(SEQ ID NO: 204)
AIDWNGRGTYYRY,
(SEQ ID NO: 205)
RINGGGITH,
(SEQ ID NO: 206)
FIDRIATTT,
(SEQ ID NO: 207)
AITWNGGTTY,
(SEQ ID NO: 208)
FISGGGTTT,
(SEQ ID NO: 209)
RITGGGSTH,
(SEQ ID NO: 210)
AISWSGGSTT,
(SEQ ID NO: 211)
AISWSGSSAG,
(SEQ ID NO: 212)
AIRWSGGRTL,
or
(SEQ ID NO: 213)
SITWNGGSTS.

In some embodiments, the single domain antibody comprises a CDR1 sequence of

(SEQ ID NO: 1)
SYRMG,
(SEQ ID NO: 2)
INVMG,
(SEQ ID NO: 3)
SNAMG,
(SEQ ID NO: 4)
SYAMG,
(SEQ ID NO: 5)
SDAMG,
(SEQ ID NO: 6)
INVMG,
(SEQ ID NO: 7)
TYRMG,
(SEQ ID NO: 8)
RYAMG,
(SEQ ID NO: 258)
FTTYRMG,
(SEQ ID NO: 259)
TYRMG,
(SEQ ID NO: 9)
FNTYAMG,
(SEQ ID NO: 10)
GLTFSSY,
(SEQ ID NO: 11)
GSIFSIN,
(SEQ ID NO: 12)
GTSVSSN,
(SEQ ID NO: 13)
GRTFSSY,
(SEQ ID NO: 14)
GSSVSSD,
(SEQ ID NO: 15)
RSIGSIN,
(SEQ ID NO: 16)
GRTFSTY,
(SEQ ID NO: 17)
GFTFTRY,
(SEQ ID NO: 18)
GRTFTTY,
(SEQ ID NO: 19)
GRTLSFNTY,
(SEQ ID NO: 20)
GLTFSSYR,
(SEQ ID NO: 21)
GSIFSINV,
(SEQ ID NO: 22)
GTSVSSNA,
(SEQ ID NO: 23)
GRTFSSYA,
(SEQ ID NO: 24)
GSSVSSDA,
(SEQ ID NO: 25)
RSIGSINV,
(SEQ ID NO: 26)
GRTFSTYR,
(SEQ ID NO: 27)
GFTFTRYA,
(SEQ ID NO: 28)
GRTFTTYR,
(SEQ ID NO: 29)
GRTLSFNTYA,
(SEQ ID NO: 154)
GLTFSSYRMG,
(SEQ ID NO: 155)
GSIFSINVMG,
(SEQ ID NO: 156)
GTSVSSNAMG,
(SEQ ID NO: 157)
GRTFSSYAMG,
(SEQ ID NO: 158)
GSSVSSDAMG,
(SEQ ID NO: 159)
RSIGSINVMG,
(SEQ ID NO: 160)
GRTFSTYRMG,
(SEQ ID NO: 161)
GFTFTRYAMG,
(SEQ ID NO: 162)
GRTFTTYRMG,
(SEQ ID NO: 163)
GRTLSFNTYAMG,
(SEQ ID NO: 164)
SSYRMG,
(SEQ ID NO: 165)
SINVMG,
(SEQ ID NO: 166)
SSNAMG,
(SEQ ID NO: 167)
SSYAMG,
(SEQ ID NO: 168)
SSDAMG,
(SEQ ID NO: 169)
SINVMG,
(SEQ ID NO: 170)
STYRMG,
(SEQ ID NO: 171)
TRYAMG,
(SEQ ID NO: 172)
TTYRMG,
(SEQ ID NO: 173)
SFNTYAMG,
(SEQ ID NO: 174)
GLTFSSYRMG,
(SEQ ID NO: 175)
GSIFSINVMG,
(SEQ ID NO: 176)
GTSVSSNAMG,
(SEQ ID NO: 177)
GRTFSSYAMG,
(SEQ ID NO: 178)
GSSVSSDAMG,
(SEQ ID NO: 179)
RSIGSINVMG,
(SEQ ID NO: 180)
GRTFSTYRMG,
(SEQ ID NO: 181)
GFTFTRYAMG,
(SEQ ID NO: 182)
GRTFTTYRMG,
or
(SEQ ID NO: 183)
GRTLSFNTYAMG.

In some embodiments, the single domain antibody provided herein comprises a CDR1 sequence, a CDR2 sequence, and a CDR3 sequence of the single domain antibody selected from the group consisting of:
a) VHH1:
   i) the CDR1 sequence of SYRMG (SEQ ID NO: 1), the CDR2 sequence of AIDWNGRGTYYRYYADSVKG (SEQ ID NO: 30), and the CDR3 sequence of GSIDLNWYGGMDY (SEQ ID NO: 60);
   ii) the CDR1 sequence of GLTFSSY (SEQ ID NO: 10), the CDR2 sequence of DWNGRGTYY (SEQ ID NO: 40) or WNGRGTY (SEQ ID NO: 260), and the CDR3 sequence of GSIDLNWYGGMDY (SEQ ID NO: 71) or SIDLNWYGGMD (SEQ ID NO: 272);
   iii) the CDR1 sequence of GLTFSSYR (SEQ ID NO: 20), the CDR2 sequence of IDWNGRGTYY (SEQ ID NO: 50) or IDWNGRGTYYR (SEQ ID NO: 270), and the CDR3 sequence of CAAGSIDLNWYGGMDY (SEQ ID NO: 82) or AAGSIDLNWYGGMDY (SEQ ID NO: 283);
   iv) the CDR1 sequence of GLTFSSYRMG (SEQ ID NO: 154), the CDR 2 sequence of AIDWNGRGTYYRYYADSVKG (SEQ ID NO: 184), and the CDR3 sequence of GSIDLNWYGGMDY (SEQ ID NO: 214);
   v) the CDR1 sequence of SSYRMG (SEQ ID NO: 164), the CDR2 sequence of FVAAIDWNGRGTYYRY (SEQ ID NO: 194), and the CDR3 sequence of AAGSIDLNWYGGMD (SEQ ID NO: 225); or
   vi) the CDR1 sequence of GLTFSSYRMG (SEQ ID NO: 174), the CDR2 sequence of AIDWNGRGTYYRY (SEQ ID NO: 204), and the CDR3 sequence of GSIDLNWYGGMDY (SEQ ID NO: 236);
b) VHH2:
   i) the CDR1 sequence of SYRMG (SEQ ID NO: 1), the CDR2 sequence of AIDWNGRGTYYRYYADSVKG (SEQ ID NO: 30), and the CDR3 sequence of TTVLTDPRVLNEYAT (SEQ ID NO: 61);
   ii) the CDR1 sequence of GLTFSSY (SEQ ID NO: 10), the CDR2 sequence of DWNGRGTYY (SEQ ID NO: 40) or WNGRGTY (SEQ ID NO: 260), and the CDR3 sequence of TTVLTDPRVLNEYAT (SEQ ID NO: 72) or TVLTDPRVLNEYA (SEQ ID NO: 273);
   iii) the CDR1 sequence of GLTFSSYR (SEQ ID NO: 20), the CDR2 sequence of IDWNGRGTYY (SEQ ID NO: 50) or IDWNGRGTYYR (SEQ ID NO: 270), and the CDR3 sequence of CAATTVLTDPRVLNEYAT (SEQ ID NO: 83) or AATTVLTDPRVLNEYAT (SEQ ID NO: 284);
   iv) the CDR1 sequence of GLTFSSYRMG (SEQ ID NO: 154), the CDR2 sequence of AIDWNGRGTYYRYYADSVKG (SEQ ID NO: 184), and the CDR3 sequence of TTVLTDPRVLNEYAT (SEQ ID NO: 215);
   v) the CDR1 sequence of SSYRMG (SEQ ID NO: 164), the CDR2 sequence of FVAAIDWNGRGTYYRY (SEQ ID NO: 194), and the CDR3 sequence of AATTVLTDPRVLNEYA (SEQ ID NO: 226); or
   vi) the CDR1 sequence of GLTFSSYRMG (SEQ ID NO: 174), the CDR2 sequence of AIDWNGRGTYYRY (SEQ ID NO: 204), and the CDR3 sequence of TTVLTDPRVLNEYAT (SEQ ID NO: 237);
c) VHH3:
   i) the CDR1 sequence of INVMG (SEQ ID NO: 2), the CDR2 sequence of RINGGGITHYAESVKG (SEQ ID NO: 31), and the CDR3 sequence of DVFGSSGYVETY (SEQ ID NO: 62);
   ii) the CDR1 sequence of GSIFSIN (SEQ ID NO: 11), the CDR2 sequence of NGGGI (SEQ ID NO: 41) or GGG (SEQ ID NO: 261), and the CDR3 sequence of DVFGSSGYVETY (SEQ ID NO: 73) or VFGSSGYVET (SEQ ID NO: 274);
   iii) the CDR1 sequence of GSIFSINV (SEQ ID NO: 21), the CDR2 sequence of INGGGIT (SEQ ID NO: 51), and the CDR3 sequence of KADVFGSSGYVETY (SEQ ID NO: 84);
   iv) the CDR1 sequence of GSIFSINVMG (SEQ ID NO: 155), the CDR2 sequence of RINGGGITHYAESVKG (SEQ ID NO: 185), and the CDR3 sequence of DVFGSSGYVETY (SEQ ID NO: 216);
   v) the CDR1 sequence of SINVMG (SEQ ID NO: 165), the CDR2 sequence of LVARINGGGITH (SEQ ID NO: 195), and the CDR3 sequence of KADVFGSSGYVET (SEQ ID NO: 227); or
   vi) the CDR1 sequence of GSIFSINVMG (SEQ ID NO: 175), the CDR2 sequence of RINGGGITH (SEQ ID NO: 205), and the CDR3 sequence of DVFGSSGYVETY (SEQ ID NO: 238);
d) VHH4:
   i) the CDR1 sequence of SNAMG (SEQ ID NO: 3), the CDR2 sequence of FIDRIATTTIATSVKG (SEQ ID NO: 32), and the CDR3 sequence of PLTAR (SEQ ID NO: 63);
   ii) the CDR1 sequence of GTSVSSN (SEQ ID NO: 12), the CDR2 sequence of DRIAT (SEQ ID NO: 42) or RIA (SEQ ID NO: 262), and the CDR3 sequence of PLTAR (SEQ ID NO: 74) or LTA (SEQ ID NO: 275);
   iii) the CDR1 sequence of GTSVSSNA (SEQ ID NO: 22), the CDR2 sequence of IDRIATT (SEQ ID NO: 52), and the CDR3 sequence of NHPLTAR (SEQ ID NO: 85);
   iv) the CDR1 sequence of GTSVSSNAMG (SEQ ID NO: 156), the CDR2 sequence of FIDRIATTTIATSVKG (SEQ ID NO: 186), and the CDR3 sequence of PLTAR (SEQ ID NO: 217);
   v) the CDR1 sequence of SSNAMG (SEQ ID NO: 166), the CDR2 sequence of WVGFIDRIATTT (SEQ ID NO: 196), and the CDR3 sequence of NHPLTA (SEQ ID NO: 228); or
   vi) the CDR1 sequence of GTSVSSNAMG (SEQ ID NO: 176), the CDR2 sequence of FIDRIATTT (SEQ ID NO: 206), and the CDR3 sequence of PLTAR (SEQ ID NO: 239);
e) VHH5:
   i) the CDR1 sequence of SYAMG (SEQ ID NO: 4), the CDR2 sequence of AITWNGGTTYYADSVKG (SEQ ID NO: 33), and the CDR3 sequence of DPFNQGY (SEQ ID NO: 64);
   ii) the CDR1 sequence of GRTFSSY (SEQ ID NO: 13), the CDR2 sequence of TWNGGT (SEQ ID NO: 43) or WNGG (SEQ ID NO: 263), and the CDR3 sequence of DPFNQGY (SEQ ID NO: 75) or PFNQG (SEQ ID NO: 276);
   iii) the CDR1 sequence of GRTFSSYA (SEQ ID NO: 23), the CDR2 sequence of ITWNGGTT (SEQ ID NO: 53), and the CDR3 sequence of AADPFNQGY (SEQ ID NO: 86);
   iv) the CDR1 sequence of GRTFSSYAMG (SEQ ID NO: 157), the CDR2 sequence of AITWNGGTTYYADSVKG (SEQ ID NO: 187), and the CDR3 sequence of DPFNQGY (SEQ ID NO: 218);
   v) the CDR1 sequence of SSYAMG (SEQ ID NO: 167), the CDR2 sequence of FVAAITWNGGTTY (SEQ ID NO: 197), and the CDR3 sequence of AADPFNQG (SEQ ID NO: 229); or
   vi) the CDR1 sequence of GRTFSSYAMG (SEQ ID NO: 177), the CDR2 sequence of AITWNGGTTY (SEQ ID NO: 207), and the CDR3 sequence of DPFNQGY (SEQ ID NO: 240);
f) VHH6:
   i) the CDR1 sequence of SDAMG (SEQ ID NO: 5), the CDR2 sequence of FISGGGTTTYADSVKG (SEQ ID NO: 34), and the CDR3 sequence of PLTSR (SEQ ID NO: 65);
   ii) the CDR1 sequence of GSSVSSD (SEQ ID NO: 14), the CDR2 sequence of SGGGT (SEQ ID NO: 44) or GGG (SEQ ID NO: 264), and the CDR3 sequence of PLTSR (SEQ ID NO: 76) or LTS (SEQ ID NO: 277);
   iii) the CDR1 sequence of GSSVSSDA (SEQ ID NO: 24), the CDR2 sequence of ISGGGTT (SEQ ID NO: 54), and the CDR3 sequence of NHPLTSR (SEQ ID NO: 87);
   iv) the CDR1 sequence of GSSVSSDAMG (SEQ ID NO: 158), the CDR2 sequence of FISGGGTTTYADSVKG (SEQ ID NO: 188), and the CDR3 sequence of PLTSR (SEQ ID NO: 219);
   v) the CDR1 sequence of SSDAMG (SEQ ID NO: 168), the CDR2 sequence of WVAFISGGGTTT (SEQ ID NO: 198), and the CDR3 sequence of NHPLTS (SEQ ID NO: 230); or
   vi) the CDR1 sequence of GSSVSSDAMG (SEQ ID NO: 178), the CDR2 sequence of FISGGGTTT (SEQ ID NO: 208), and the CDR3 sequence of PLTSR (SEQ ID NO: 241);
g) VHH7:
   i) the CDR1 sequence of INVMG (SEQ ID NO: 6), the CDR2 sequence of RITGGGSTHYAESVKG (SEQ ID NO: 35), and the CDR3 sequence of MVNPIITAWGTIGVREIPDYDY (SEQ ID NO: 66);
   ii) the CDR1 sequence of RSIGSIN (SEQ ID NO: 15), the CDR2 sequence of TGGGS (SEQ ID NO: 45) or GGG (SEQ ID NO: 265), and the CDR3 sequence of MVNPIITAWGTIGVREIPDYDY (SEQ ID NO: 77) or VNPIITAWGTIGVREIPDYD (SEQ ID NO: 278);
   iii) the CDR1 sequence of RSIGSINV (SEQ ID NO: 25), the CDR2 sequence of ITGGGST (SEQ ID NO: 55), and the CDR3 sequence of ASMVNPIITAWGTIGVREIPDYDY (SEQ ID NO: 88);
   iv) the CDR1 sequence of RSIGSINVMG (SEQ ID NO: 159), the CDR2 sequence of RITGGGSTHYAESVKG (SEQ ID NO: 189), and the CDR3 sequence of MVNPIITAWGTIGVREIPDYDY (SEQ ID NO: 220);
   v) the CDR1 sequence of SINVMG (SEQ ID NO: 169), the CDR2 sequence of LVARITGGGSTH (SEQ ID NO: 199), and the CDR3 sequence of ASMVNPIITAWGTIGVREIPDYD (SEQ ID NO: 231); or
   vi) the CDR1 sequence of RSIGSINVMG (SEQ ID NO: 179), the CDR2 sequence of RITGGGSTH (SEQ ID NO: 209), and the CDR3 sequence of MVNPIITAWGTIGVREIPDYDY (SEQ ID NO: 242);
h) VHH9:
   i) the CDR1 sequence of TYRMG (SEQ ID NO: 7), the CDR2 sequence of AISWSGGSTTYADPVKG (SEQ ID NO: 36), and the CDR3 sequence of DQRGY (SEQ ID NO: 67) or QRGY (SEQ ID NO: 271);
   ii) the CDR1 sequence of GRTFSTY (SEQ ID NO: 16), the CDR2 sequence of SWSGGS (SEQ ID NO: 46) or WSGG (SEQ ID NO: 266), and the CDR3 sequence of DQRGY (SEQ ID NO: 78) or RG (SEQ ID NO: 279);
   iii) the CDR1 sequence of GRTFSTYR (SEQ ID NO: 26), the CDR2 sequence of ISWSGGST (SEQ ID NO: 56), and the CDR3 sequence of NDQRGY (SEQ ID NO: 89);
   iv) the CDR1 sequence of GRTFSTYRMG (SEQ ID NO: 160), the CDR2 sequence of AISWSGGSTTYADPVKG (SEQ ID NO: 190), and the CDR3 sequence of QRGY (SEQ ID NO: 221);
   v) the CDR1 sequence of STYRMG (SEQ ID NO: 170), the CDR2 sequence of FVAAISWSGGSTT (SEQ ID NO: 200), and the CDR3 sequence of NDQRG (SEQ ID NO: 232); or
   vi) the CDR1 sequence of GRTFSTYRMG (SEQ ID NO: 180), the CDR2 sequence of AISWSGGSTT (SEQ ID NO: 210), and the CDR3 sequence of QRGY (SEQ ID NO: 243);
i) VHH10:
   i) the CDR1 sequence of RYAMG (SEQ ID NO: 8), the CDR2 sequence of AISWSGSSAGYGDSVKG (SEQ ID NO: 37), and the CDR3 sequence of DPFNQGY (SEQ ID NO: 68);
   ii) the CDR1 sequence of GFTFTRY (SEQ ID NO: 17), the CDR2 sequence of SWSGSS (SEQ ID NO: 47) or WSGS (SEQ ID NO: 267), and the CDR3 sequence of DPFNQGY (SEQ ID NO: 79) or PFNQG (SEQ ID NO: 280);
   iii) the CDR1 sequence of GFTFTRYA (SEQ ID NO: 27), the CDR2 sequence of ISWSGSSA (SEQ ID NO: 57), and the CDR3 sequence of AADPFNQGY (SEQ ID NO: 90);
   iv) the CDR1 sequence of GFTFTRYAMG (SEQ ID NO: 161), the CDR2 sequence of AISWSGSSAGYGDSVKG (SEQ ID NO: 191), and the CDR3 sequence of DPFNQGY (SEQ ID NO: 222);
   v) the CDR1 sequence of TRYAMG (SEQ ID NO: 171), the CDR2 sequence of FVAAISWSGSSAG (SEQ ID NO: 201), and the CDR3 sequence of AADPFNQG (SEQ ID NO: 233); or
   vi) the CDR1 sequence of GFTFTRYAMG (SEQ ID NO: 181), the CDR2 sequence of AISWSGSSAG (SEQ ID NO: 211), and the CDR3 sequence of DPFNQGY (SEQ ID NO: 244);
j) VHH11:
   i) the CDR1 sequence of FTTYRMG (SEQ ID NO: 258) or TYRMG (SEQ ID NO: 259), the CDR2 sequence of AIRWSGGRTLYADSVKG (SEQ ID NO: 38), and the CDR3 sequence of DLAEYSGTYSSPADSPAGYDY (SEQ ID NO: 69);
   ii) the CDR1 sequence of GRTFTTY (SEQ ID NO: 18), the CDR2 sequence of RWSGGR (SEQ ID NO: 48) or WSGG (SEQ ID NO: 268), and the CDR3 sequence of DLAEYSGTYSSPADSPAGYDY (SEQ ID NO: 80) or LAEYSGTYSSPADSPAGYD (SEQ ID NO: 281);
   iii) the CDR1 sequence of GRTFTTYR (SEQ ID NO: 28), the CDR2 sequence of IRWSGGRT (SEQ ID NO: 58), and the CDR3 sequence of AADLAEYSGTYSSPADSPAGYDY (SEQ ID NO: 91);
   iv) the CDR1 sequence of GRTFTTYRMG (SEQ ID NO: 162), the CDR2 sequence of AIRWSGGRTLYADSVKG (SEQ ID NO: 192), and the CDR3 sequence of DLAEYSGTYSSPADSPAGYDY (SEQ ID NO: 223);
   v) the CDR1 sequence of TTYRMG (SEQ ID NO: 172), the CDR2 sequence of FVAAIRWSGGRTL (SEQ ID NO: 202), and the CDR3 sequence of AADLAEYSGTYSSPADSPAGYD (SEQ ID NO: 234); or
   vi) the CDR1 sequence of GRTFTTYRMG (SEQ ID NO: 182), the CDR2 sequence of AIRWSGGRTL (SEQ ID NO: 212), and the CDR3 sequence of DLAEYSGTYSSPADSPAGYDY (SEQ ID NO: 245); and
k) VHH12:
   i) the CDR1 sequence of FNTYAMG (SEQ ID NO: 9), the CDR2 sequence of SITWNGGSTSYADSVKG (SEQ ID NO: 39), and the CDR3 sequence of ARYYVSGTYFPANY (SEQ ID NO: 70);
   ii) the CDR1 sequence of GRTLSFNTY (SEQ ID NO: 19), the CDR2 sequence of TWNGGS (SEQ ID NO: 49) or WNGG (SEQ ID NO: 269), and the CDR3 sequence of ARYYVSGTYFPANY (SEQ ID NO: 81) or RYYVSGTYFPAN (SEQ ID NO: 282);
   iii) the CDR1 sequence of GRTLSFNTYA (SEQ ID NO: 29), the CDR2 sequence of ITWNGGST (SEQ ID NO: 59), and the CDR3 sequence of AAARYYVSGTYFPANY (SEQ ID NO: 92);
   iv) the CDR1 sequence of GRTLSFNTYAMG (SEQ ID NO: 163), the CDR2 sequence of SITWNGGSTSYADSVKG (SEQ ID NO: 193), and the CDR3 sequence of ARYYVSGTYFPANY (SEQ ID NO: 224);
   v) the CDR1 sequence of SFNTYAMG (SEQ ID NO: 173), the CDR2 sequence of FVASITWNGGSTS (SEQ ID NO: 203), and the CDR3 sequence of AAARYYVSGTYFPAN (SEQ ID NO: 235); or
   vi) the CDR1 sequence of GRTLSFNTYAMG (SEQ ID NO: 183), the CDR2 sequence of SITWNGGSTS (SEQ ID NO: 213), and the CDR3 sequence of ARYYVSGTYFPANY (SEQ ID NO: 246).

In some embodiments, the single domain antibody comprises a framework derived from the framework of any of the single domain antibodies comprising the sequences of

(SEQ ID NO: 93)
QVQLVESGGGLVQAGGSLKLACAAPGLTFSSYRMGWFRQAPGQEREFVA
AIDWNGRGTYYRYYADSVKGRSTISRDNAKNTMYLQMNSLKPEDTAVYY
CAAGSIDLNWYGGMDYWGQGTQVTVSS,
(SEQ ID NO: 94)
EVQVVESGGGLVQAGGSLKLACAAPGLTFSSYRMGWFRQAPGQEREFVA
AIDWNGRGTYYRYYADSVKGRSTISRDNAKNTVYLQMNSLKPEDTAVYY
CAATTVLTDPRVLNEYATWGQGTQVTVSS,
(SEQ ID NO: 95)
EVQVVESGGGLVQAGGSLRLSCAVSGTSVSSNAMGWYRQAPGKQREWVG
FIDRIATTTIATSVKGRFAITRDNAKNTVYLQMSGLKPEDTAVYYCNHP
LTARWGQGTQVTVSS,
(SEQ ID NO: 96)
QVQLVESGGGLVQAGGSLRLSCAASGRTFSSYAMGWFRQAPGKEREFVA
AITWNGGTTYYADSVKGRFTISRDNAKNTVYLQMNSLKPEDTAVYYCAA
DPFNQGYWGQGTQVTVSS,
(SEQ ID NO: 97)
EVQLVESGGGLVQAGGSLRLSCAVSGSSVSSDAMGWYRQAPGNQRAWVA
FISGGGTTTYADSVKGRFTISRDNTKNTVYLHMNSLKPEDTAVYYCNHP
LTSRWGQGTQVTVSS,
(SEQ ID NO: 98)
EVQVVESGGGLVQAGGSLRLACVASRSIGSINVMGWYRQAPGKQRDLVA
RITGGGSTHYAESVKGRFTISRDNAKNTVYLQMNSLEPEDTAVYYCASM
VNPIITAWGTIGVREIPDYDYWGQGTQVTVSS,
(SEQ ID NO: 99)
QVQLVESGGGLVQAGGSLRLSCAVSGRTFSTYRMGWFRQAPGKERSFVA
AISWSGGSTTYADPVKGRFTISRDNAKNTVYLRMNSLKPEDTAVYYCND
QRGYWGQGTLVTVSS,
(SEQ ID NO: 100)
EVQVVESGGGLVQAGGSLRLSCAASGFTFTRYAMGWFRQAPGKERSFVA
AISWSGSSAGYGDSVKGRFTISRDNAKNTLYLQMNSLKPEDTAVYYCAA
DPFNQGYWGQGTQVTVSS,
(SEQ ID NO: 101)
EVQVVESGGGLVQAGGSLRLSCAASGRTFTTYRMGWFRQAPGKEREFVA
AIRWSGGRTLYADSVKGRFTISRDNAKNTAYLQMNNLRPEDTAVYYCAA
DLAEYSGTYSSPADSPAGYDYWGQGTQVTVSS,
or
(SEQ ID NO: 102)
QVQLVETGGGLVQAGDSLRLSCAASGRTLSFNTYAMGWFRQAPGKEREF
VASITWNGGSTSYADSVKGRFTITRDNAKNTATLRMNSLQPDDTAVYYC
AAARYYVSGTYFPANYWGQGTQVTVSS.

In some embodiments, the single domain antibody comprises a framework comprising sequence having at least 75%, 80%, 85%, 90%, 95%, 96%, 97%, 98%, 99%, or 100% sequence identity with the sequence of

(SEQ ID NO: 93)
QVQLVESGGGLVQAGGSLKLACAAPGLTFSSYRMGWFRQAPGQEREFVA
AIDWNGRGTYYRYYADSVKGRSTISRDNAKNTMYLQMNSLKPEDTAVYY
CAAGSIDLNWYGGMDYWGQGTQVTVSS,
(SEQ ID NO: 94)
EVQVVESGGGLVQAGGSLKLACAAPGLTFSSYRMGWFRQAPGQEREFVA
AIDWNGRGTYYRYYADSVKGRSTISRDNAKNTVYLQMNSLKPEDTAVYY
CAATTVLTDPRVLNEYATWGQGTQVTVSS,
(SEQ ID NO: 95)
EVQVVESGGGLVQAGGSLRLSCAVSGTSVSSNAMGWYRQAPGKQREWVG
FIDRIATTTIATSVKGRFAITRDNAKNTVYLQMSGLKPEDTAVYYCNHP
LTARWGQGTQVTVSS,
(SEQ ID NO: 96)
QVQLVESGGGLVQAGGSLRLSCAASGRTFSSYAMGWFRQAPGKEREFVA
AITWNGGTTYYADSVKGRFTISRDNAKNTVYLQMNSLKPEDTAVYYCAA
DPFNQGYWGQGTQVTVSS,
(SEQ ID NO: 97)
EVQLVESGGGLVQAGGSLRLSCAVSGSSVSSDAMGWYRQAPGNQRAWVA
FISGGGTTTYADSVKGRFTISRDNTKNTVYLHMNSLKPEDTAVYYCNHP
LTSRWGQGTQVTVSS,
(SEQ ID NO: 98)
EVQVVESGGGLVQAGGSLRLACVASRSIGSINVMGWYRQAPGKQRDLVA
RITGGGSTHYAESVKGRFTISRDNAKNTVYLQMNSLEPEDTAVYYCASM
VNPIITAWGTIGVREIPDYDYWGQGTQVTVSS,
(SEQ ID NO: 99)
QVQLVESGGGLVQAGGSLRLSCAVSGRTFSTYRMGWFRQAPGKERSFVA
AISWSGGSTTYADPVKGRFTISRDNAKNTVYLRMNSLKPEDTAVYYCND
QRGYWGQGTLVTVSS,
(SEQ ID NO: 100)
EVQVVESGGGLVQAGGSLRLSCAASGFTFTRYAMGWFRQAPGKERSFVA
AISWSGSSAGYGDSVKGRFTISRDNAKNTLYLQMNSLKPEDTAVYYCAA
DPFNQGYWGQGTQVTVSS,
(SEQ ID NO: 101)
EVQVVESGGGLVQAGGSLRLSCAASGRTFTTYRMGWFRQAPGKEREFVA
AIRWSGGRTLYADSVKGRFTISRDNAKNTAYLQMNNLRPEDTAVYYCAA
DLAEYSGTYSSPADSPAGYDYWGQGTQVTVSS,
or
(SEQ ID NO: 102)
QVQLVETGGGLVQAGDSLRLSCAASGRTLSFNTYAMGWFRQAPGKEREF
VASITWNGGSTSYADSVKGRFTITRDNAKNTATLRMNSLQPDDTAVYYC
AAARYYVSGTYFPANYWGQGTQVTVSS.

In some embodiments, the single domain antibody is comprised of a sequence having at least 75%, 80%, 85%, 90%, 95%, 96%, 97%, 98%, 99%, or 100% sequence identity with the sequence of

(SEQ ID NO: 93)
QVQLVESGGGLVQAGGSLKLACAAPGLTFSSYRMGWFRQAPGQEREFVA
AIDWNGRGTYYRYYADSVKGRSTISRDNAKNTMYLQMNSLKPEDTAVYY
CAAGSIDLNWYGGMDYWGQGTQVTVSS,
(SEQ ID NO: 94)
EVQVVESGGGLVQAGGSLKLACAAPGLTFSSYRMGWFRQAPGQEREFVA
AIDWNGRGTYYRYYADSVKGRSTISRDNAKNTVYLQMNSLKPEDTAVYY
CAATTVLTDPRVLNEYATWGQGTQVTVSS,
(SEQ ID NO: 95)
EVQVVESGGGLVQAGGSLRLSCAVSGTSVSSNAMGWYRQAPGKQREWVG
FIDRIATTTIATSVKGRFAITRDNAKNTVYLQMSGLKPEDTAVYYCNHP
LTARWGQGTQVTVSS,
(SEQ ID NO: 96)
QVQLVESGGGLVQAGGSLRLSCAASGRTFSSYAMGWFRQAPGKEREFVA
AITWNGGTTYYADSVKGRFTISRDNAKNTVYLQMNSLKPEDTAVYYCAA
DPFNQGYWGQGTQVTVSS,
(SEQ ID NO: 97)
EVQLVESGGGLVQAGGSLRLSCAVSGSSVSSDAMGWYRQAPGNQRAWVA
FISGGGTTTYADSVKGRFTISRDNTKNTVYLHMNSLKPEDTAVYYCNHP
LTSRWGQGTQVTVSS,
(SEQ ID NO: 98)
EVQVVESGGGLVQAGGSLRLACVASRSIGSINVMGWYRQAPGKQRDLVA
RITGGGSTHYAESVKGRFTISRDNAKNTVYLQMNSLEPEDTAVYYCASM
VNPIITAWGTIGVREIPDYDYWGQGTQVTVSS,
(SEQ ID NO: 99)
QVQLVESGGGLVQAGGSLRLSCAVSGRTFSTYRMGWFRQAPGKERSFVA
AISWSGGSTTYADPVKGRFTISRDNAKNTVYLRMNSLKPEDTAVYYCND
QRGYWGQGTLVTVSS,
(SEQ ID NO: 100)
EVQVVESGGGLVQAGGSLRLSCAASGFTFTRYAMGWFRQAPGKERSFVA
AISWSGSSAGYGDSVKGRFTISRDNAKNTLYLQMNSLKPEDTAVYYCAA
DPFNQGYWGQGTQVTVSS,
(SEQ ID NO: 101)
EVQVVESGGGLVQAGGSLRLSCAASGRTFTTYRMGWFRQAPGKEREFVA
AIRWSGGRTLYADSVKGRFTISRDNAKNTAYLQMNNLRPEDTAVYYCAA
DLAEYSGTYSSPADSPAGYDYWGQGTQVTVSS,
or
(SEQ ID NO: 102)
QVQLVETGGGLVQAGDSLRLSCAASGRTLSFNTYAMGWFRQAPGKEREF
VASITWNGGSTSYADSVKGRFTITRDNAKNTATLRMNSLQPDDTAVYYC
AAARYYVSGTYFPANYWGQGTQVTVSS.

In some embodiments, the single domain antibody is genetically fused or chemically conjugated to the agent.

In some embodiments, the single domain antibody provided herein further comprises a linker between the single domain antibody and the agent. In some embodiments, the linker is a polypeptide. In some embodiments, the linker is a flexible linker comprising a sequence selected from the group consisting of EPKTPKPQPQPQLQPQPNPTTESKSPK (SEQ ID NO: 130), (EAAAK)n (SEQ ID NO: 147), (GGGGS)n (SEQ ID NO: 148) and (GGGS)n (SEQ ID NO: 149), wherein n is an integer from 1 to 20.

In some embodiments, the single domain antibody is chemically-conjugated to the agent. In other embodiments, the single domain antibody is non-covalently bound to the agent.

In some embodiments, the method provided herein does not inhibit pIgR-mediated transcytosis of IgA.

In some embodiments, the single domain antibody comprises a CDR1 sequence of

(SEQ ID NO: 3)
SNAMG,
(SEQ ID NO: 6)
INVMG,
(SEQ ID NO: 7)
TYRMG,
(SEQ ID NO: 8)
RYAMG,
(SEQ ID NO: 258)
FTTYRMG,
(SEQ ID NO: 259)
TYRMG,
(SEQ ID NO: 9)
FNTYAMG,
(SEQ ID NO: 12)
GTSVSSN,
(SEQ ID NO: 13)
GRTFSSY,
(SEQ ID NO: 15)
RSIGSIN,
(SEQ ID NO: 16)
GRTFSTY,
(SEQ ID NO: 17)
GFTFTRY,
(SEQ ID NO: 18)
GRTFTTY,
(SEQ ID NO: 19)
GRTLSFNTY,
(SEQ ID NO: 22)
GTSVSSNA,
(SEQ ID NO: 25)
RSIGSINV,
(SEQ ID NO: 26)
GRTFSTYR,
(SEQ ID NO: 27)
GFTFTRYA,
(SEQ ID NO: 28)
GRTFTTYR,
(SEQ ID NO: 29)
GRTLSFNTYA,
(SEQ ID NO: 156)
GTSVSSNAMG,
(SEQ ID NO: 159)
RSIGSINVMG,
(SEQ ID NO: 160)
GRTFSTYRMG,
(SEQ ID NO: 161)
GFTFTRYAMG,
(SEQ ID NO: 162)
GRTFTTYRMG,
(SEQ ID NO: 163)
GRTLSFNTYAMG,
(SEQ ID NO: 166)
SSNAMG,
(SEQ ID NO: 169)
SINVMG,
(SEQ ID NO: 170)
STYRMG,
(SEQ ID NO: 171)
TRYAMG,
(SEQ ID NO: 172)
TTYRMG,
(SEQ ID NO: 173)
SFNTYAMG,
(SEQ ID NO: 176)
GTSVSSNAMG,
(SEQ ID NO: 179)
RSIGSINVMG,
(SEQ ID NO: 180)
GRTFSTYRMG,
(SEQ ID NO: 181)
GFTFTRYAMG,
(SEQ ID NO: 182)
GRTFTTYRMG,
or
(SEQ ID NO: 183)
GRTLSFNTYAMG.

In some embodiments, the single domain antibody comprises a CDR2 sequence of

(SEQ ID NO: 32)
FIDRIATTTIATSVKG,
(SEQ ID NO: 35)
RITGGGSTHYAESVKG,
(SEQ ID NO: 36)
AISWSGGSTTYADPVKG,
(SEQ ID NO: 37)
AISWSGSSAGYGDSVKG,
(SEQ ID NO: 38)
AIRWSGGRTLYADSVKG,
(SEQ ID NO: 39)
SITWNGGSTSYADSVKG,
(SEQ ID NO: 42)
DRIAT,
(SEQ ID NO: 262)
RIA,
(SEQ ID NO: 45)
TGGGS,
(SEQ ID NO: 265)
GGG,
(SEQ ID NO: 46)
SWSGGS,
(SEQ ID NO: 266)
WSGG,
(SEQ ID NO: 47)
SWSGSS,
(SEQ ID NO: 267)
WSGS,
(SEQ ID NO: 48)
RWSGGR,
(SEQ ID NO: 268)
WSGG,
(SEQ ID NO: 49)
TWNGGS,
(SEQ ID NO: 269)
WNGG,
(SEQ ID NO: 52)
IDRIATT,
(SEQ ID NO: 55)
ITGGGST,
(SEQ ID NO: 56)
ISWSGGST,
(SEQ ID NO: 57)
ISWSGSSA,
(SEQ ID NO: 58)
IRWSGGRT,
(SEQ ID NO: 59)
ITWNGGST,
(SEQ ID NO: 186)
FIDRIATTTIATSVKG,
(SEQ ID NO: 189)
RITGGGSTHYAESVKG,
(SEQ ID NO: 190)
AISWSGGSTTYADPVKG,
(SEQ ID NO: 191)
AISWSGSSAGYGDSVKG,
(SEQ ID NO: 192)
AIRWSGGRTLYADSVKG,
(SEQ ID NO: 193)
SITWNGGSTSYADSVKG,
(SEQ ID NO: 196)
WVGFIDRIATTT,
(SEQ ID NO: 199)
LVARITGGGSTH,
(SEQ ID NO: 200)
FVAAISWSGGSTT,
(SEQ ID NO: 201)
FVAAISWSGSSAG,
(SEQ ID NO: 202)
FVAAIRWSGGRTL,
(SEQ ID NO: 203)
FVASITWNGGSTS,
(SEQ ID NO: 206)
FIDRIATTT,
(SEQ ID NO: 209)
RITGGGSTH,
(SEQ ID NO: 210)
AISWSGGSTT,
(SEQ ID NO: 211)
AISWSGSSAG,
(SEQ ID NO: 212)
AIRWSGGRTL,
or
(SEQ ID NO: 213)
SITWNGGSTS.

In some embodiments, the single domain antibody comprises a CDR3 sequence of

(SEQ ID NO: 63)
PLTAR,
(SEQ ID NO: 66)
MVNPIITAWGTIGVREIPDYDY,
(SEQ ID NO: 67)
DQRGY,
(SEQ ID NO: 271)
QRGY,
(SEQ ID NO: 68)
DPFNQGY,
(SEQ ID NO: 69)
DLAEYSGTYSSPADSPAGYDY,
(SEQ ID NO: 70)
ARYYVSGTYFPANY,
(SEQ ID NO: 74)
PLTAR,
(SEQ ID NO: 275)
LTA,
(SEQ ID NO: 77)
MVNPIITAWGTIGVREIPDYDY,
(SEQ ID NO: 278)
VNPIITAWGTIGVREIPDYD,
(SEQ ID NO: 78)
DQRGY,
(SEQ ID NO: 279)
RG,
(SEQ ID NO: 79)
DPFNQGY,
(SEQ ID NO: 280)
PFNQG,
(SEQ ID NO: 80)
DLAEYSGTYSSPADSPAGYDY,
(SEQ ID NO: 281)
LAEYSGTYSSPADSPAGYD,
(SEQ ID NO: 81)
ARYYVSGTYFPANY,
(SEQ ID NO: 282)
RYYVSGTYFPAN,
(SEQ ID NO: 85)
NHPLTAR,
(SEQ ID NO: 88)
ASMVNPIITAWGTIGVREIPDYD,
(SEQ ID NO: 89)
NDQRGY,
(SEQ ID NO: 90)
AADPFNQGY,
(SEQ ID NO: 91)
AADLAEYSGTYSSPADSPAGYDY,
(SEQ ID NO: 92)
AAARYYVSGTYFPANY,
(SEQ ID NO: 217)
PLTAR,
(SEQ ID NO: 220)
MVNPIITAWGTIGVREIPDYDY,
(SEQ ID NO: 221)
QRGY,
(SEQ ID NO: 222)
DPFNQGY,
(SEQ ID NO: 223)
DLAEYSGTYSSPADSPAGYDY,
(SEQ ID NO: 224)
ARYYVSGTYFPANY,
(SEQ ID NO: 228)
NHPLTA,
(SEQ ID NO: 231)
ASMVNPIITAWGTIGVREIPDYD,
(SEQ ID NO: 232)
NDQRG,
(SEQ ID NO: 233)
AADPFNQG,
(SEQ ID NO: 234)
AADLAEYSGTYSSPADSPAGYD,
(SEQ ID NO: 235)
AAARYYVSGTYFPAN,
(SEQ ID NO: 239)
PLTAR,
(SEQ ID NO: 242)
MVNPIITAWGTIGVREIPDYDY,
(SEQ ID NO: 243)
QRGY,
(SEQ ID NO: 244)
DPFNQGY,
(SEQ ID NO: 245)
DLAEYSGTYSSPADSPAGYDY,
or
(SEQ ID NO: 246)
ARYYVSGTYFPANY.

### 4. BRIEF DESCRIPTION OF THE DRAWINGS

The foregoing will be apparent from the following more particular description of example embodiments, as illustrated in the accompanying drawings.

This patent application file contains at least one drawing executed in color. Copies of this patent application with color drawing(s) will be provided by the Office upon request and payment of the necessary fee.

FIGS. 1A and 1B are schematics showing the pathway of pIgR-mediated bidirectional transcytosis. FIG. 1A shows that molecules binding to the secretory component (domains 1-5) of the pIgR ectodomain, such as dimeric IgA (natural ligand) or VHH (artificial pIgR ligand), can transcytose the epithelial cell from the basolateral to the apical direction and reach the mucosal lumen from blood. This secretory component-mediated forward transport can be used for delivering molecules to the mucosal lumen from systemic circulation. Described herein are VHH molecules that bind to the secretory component and transcytose from the basolateral to the apical side of the epithelium. FIG. 1B shows that molecules binding to the stalk region of the pIgR ectodomain (any artificial ligand) can transcytose the epithelial cell from the apical to the basolateral direction and reach the blood from mucosal lumen. This stalk-mediated reverse transport can be used for delivering molecules to systemic circulation following oral consumption.

FIG. 2 illustrates data on epitope mapping of pIgR binders. Nine HIS-tagged pIgR constructs (D1, D2, D3, D4, D5, D1-D2, D2-D3, D3-D4 and D4-D5 were expressed and purified from HEK293 cells using immobilized metal ion affinity chromatography. Because the expression and purification yield were very low for two constructs (D4 and D3-D4), these were not used for binding studies. The heat map of FIG. 2 shows the binding of VHH-mono-Fc molecules to immobilized pIgR constructs in electrochemiluminescence units. K_{D} values for all positive interactions were measured by bio-layer interferometry. The heat map of FIG. 2 indicates that the epitopes of VHH2 and VHH3 are primarily contained within hpIgR domain-1, the epitopes of VHH4 and VHH6 are primarily contained within hpIgR domain-2, and the epitopes of other six VHHs are primarily contained within hpIgR domains 4-5.

FIGS. 3A-3B illustrate data on the effect of VHH on IgA binding to hpIgR-ECD. FIG. 3A shows K_{D} values for full-length hpIgR ECD binding to immobilized VHH-mono-Fc in the absence (blue) and presence (red) of dIgA2. FIG. 3B shows the K_{D} values for immobilized dIgA2 binding to hpIgR ectodomain with and without the presence of VHH-mono-Fc molecules. dIgA2 was immobilized using amine-reactive biosensors, and the binding of pIgR and pIgR-VHH complexes were measured by bio-layer interferometry. Three molecules (VHH2, VHH3 and VHH5) had a negative effect on IgA binding to pIgR. Other VHH molecules display a small positive effect on IgA binding to pIgR.

FIG. 4 depicts the results of assays on the transcytosis activity of VHH-mono-Fc molecules. The top panel is a schematic of the EpiAirway primary human lung tissue model used for assaying VHH transcytosis. The meso-scale discovery (MSD) assay was developed to quantify the amount of VHH present in the basolateral and apical chambers before and after transcytosis. A biotinylated anti-VHH antibody was used to capture VHH-mono-Fc molecules on streptavidin plates and a ruthenylated anti-human-Fc antibody was used as a detection antibody. The bottom panel is a graph showing the amount of VHH present in the apical mucus 24 hours post VHH treatment. Five VHH molecules (VHH2, VHH6, VHH9, VHH11 and VHH12) showed greater than 20-fold increase in their mucosal amount relative to control VHH molecules (VHH1, VHH13, and VHH14).

FIG. 5 illustrates data showing tracking pIgR and VHH across the primary human lung tissue model. The left panel of FIG. 5 is a heatmap showing the amount of pIgR retained on the EpiAirway primary human lung tissue model following transcytosis. The right panel of FIG. 5 is a heatmap showing the amount of VHH retained on the EpiAirway primary human lung tissue model following transcytosis. Following 48 hours post-treatment, tissue samples were fixed, permeabilized and stained for hpIgR and VHH. The amount of pIgR and VHH retained across the tissue model was quantified by indirect immunofluorescence using Opera Phenix confocal laser microscopy. FIG. 5 shows that VHHs displayed distinct profiles of pIgR and VHH distribution across the tissue depth dimension. FIG. 5 also shows that Among the five VHHs that showed potent transcytosis, VHH2, VHH9 and VHH12-treated tissue models showed higher VHH staining near the apical surface than the other VHHs. VHH6-treated model showed the lowest staining for both VHH and pIgR across the tissue thickness. Imaging studies corroborated transcytosis results and showed colocalization of hpIgR and VHH, especially closer to the apical epithelium.

FIG. 6A is a schematic showing the structure of pIgR.

FIG. 6B is a schematic showing a mechanism of pIgR-mediated transport. Figure adapted from Kaetzel, Curr. Biol., 2001, 11(1):R35-38.

FIG. 7 shows the expression of pIgR in various organs.

FIG. 8 shows selection criteria used to assess VHH molecules that were generated from mpIgR antigen.

FIG. 9 shows selection criteria used to assess VHH molecules that were generated from hpIgR antigen.

FIG. 10 shows the results of an assay for ability of VHH molecules to bind to MDCK cells expressing pIgR.

FIG. 11 shows the expression of hpIgR on MDCK cells. Staining shows hpIgR located on the surface and interior of the monolayer of MDCK cells. The distribution of hpIgR staining within the monolayer is not uniform. Initial experiments show hpIgR receptor density at about 6000 on the surface per cell. The blue color indicates Hoechst stain for nucleus, the green color indicates anti-pIgR antibody staining, and the red indicates anti-Rab5 staining.

FIGS. 12A-12B show the results of a VHH transcytosis assay using MDCK-hpIgR cells, as described in Example 3. Apical VHH amounts at 0, 24, and 48 hours are shown in FIG. 12B, left panel. Fold increase in apical VHH amounts at 24 hours relative to a control VHH is shown in FIG. 12B, right panel.

FIG. 12C shows transcytosis activity of VHH-mono-Fc molecules across MDCK-hpIgR monolayers from the basolateral to the apical chamber. Fold increase in apical VHH amounts at 24 hours relative to control VHH is shown.

FIG. 13 shows sequence characteristics of a set of VHH molecules, with regions of highly conserved sequence similarity are shown (SEQ ID NOS.: 93-95, 97-103 and 247-249).

FIG. 14 is a chart summarizing the purification of VHH molecules.

FIG. 15 shows the results for A-SEC purification of VHH molecules.

FIG. 16 shows the results for SEC-MALS analysis of VHH molecules.

FIG. 17 shows the results of a thermal stability assay of VHH molecules by differential scanning fluorimetry (DSF).

FIG. 18 depicts the EpiAirway human tissue model.

FIG. 19 shows the results of a VHH transcytosis assay using the EpiAirway model. The left panel shows a heat map of the amount of each tested VHH in the apical mucus at 0, 24 and 48 hours. Electrochemiluminescence (ECLU) unites obtained from the MSD assay was plotted as a heat map. The top right panel shows the amount of VHH in the apical mucus at 24 hours, and bottom right panel shows the fold increase of VHH over control in the apical mucus. The top right panel shows that five VHHs (VHH2, VHH6, VHH9, VHH11 and VHH12) showed >20-fold increase in their mucosal amount relative to control VHH molecules, and also that VHH12 showed 38-fold increase in mucus relative to control VHH and displayed the highest transcytosis activity.

FIG. 20 shows the results of IgA transcytosis assay using the EpiAirway model. FIG. 20 shows that VHH2 and VHH12-treated tissue samples stained strongly for VHH and colocalized with pIgR relative to VHH3 and VHH14 (negative control).

FIG. 21 shows colocalization of hpIgR and VHH.

FIG. 22 shows 3D reconstruction shows localization of hpIgR and VHH to the apical surface of the EpiAirway model.

FIG. 23 shows that the EpiAirway tissue model is on a slanted membrane.

FIG. 24 illustrates a strategy for Opera Phenix imaging and analysis to overcome slanted tissue issues with EpiAirway tissue model.

FIG. 25 shows the crystal structure of unliganded hpIgR in an inactive conformation. The figure is adapted from Stadtmueller et al., Elife, Mar. 4, 2016, e10640.

FIG. 26 shows structure of pIgR:IgA complex by constrained scattering modeling. The figure is adapted from Bonner et al., J. Biol. Chem., 2009, 284(8):5077-87.

FIG. 27A shows a structural model for IgA transcytosis. The figure is adapted from Stadtmueller et al., Elife, Mar. 4, 2016, e10640.

FIG. 27B shows a schematic of pIgR-mediated dimeric IgA transport across the mucosal epithelial barrier. (1) IgA production by plasma cells and IgA dimerization; (2) Binding of dimeric IgA (dIgA) to pIgR ECD on the basolateral side of the epithelium (pIgR-dIgA interactions are mediated by domains 1 and 5 of pIgR and Fc and J chains of dIgA); (3) pIgR-mediated transcytosis of dimeric IgA (clathrin-mediated endocytosis drives the basolateral to apical transport, and upon reaching the apical side, pIgR ECD is proteolytically cleaved and released into mucus along with IgA. Mucosal IgA in complex with secreted pIgR ECD (secretory component) is termed as secretory IgA (sIgA)); and (4) Neutralization of mucosal antigens by sIgA.

FIGS. 28A-28D show the effect of IgA on VHH binding to hpIgR.

FIG. 29 shows the results of domain-level epitope mapping of pIgR binders VHH1, VHH2, VHH3, VHH4, VHH5, VHH6, VHH7, VHH9, VHH10, VHH11 and VHH12. The top panel cartoon is adapted from Stadtmueller et al., Elife, Mar. 4, 2016, e10640.

FIG. 30A shows binding kinetics for hpIgR D2 binders.

FIG. 30B shows binding kinetics for hpIgR D4-D5 binders.

FIG. 31 shows properties of VHH2 and VHH3 (SEQ ID NOS.: 93-95).

FIG. 32A illustrates structure of domains and sequences of hpIgR and shows that D1 is necessary for IgA binding to hpIgR. The figure is adapted from Stadtmueller et al., Elife, Mar. 4, 2016, e10640 (SEQ ID NOS.: 250-252).

FIG. 32B shows the structure of secretory IgA1 (sIgA1), the complex between dimeric IgA and secretory component, obtained by constrained modelling of solution scattering and AUC information (created from PDB ID 3CHN). Heavy chain is shown in orange, light chain is shown in green, J chain is shown in pink and secretory component is shown in teal. The figure is adapted from Bonner et al., Mucosal Immunol., 2:74-84 (2009).

FIGS. 33A-33D show the results of VHH/IgA competition studies of Example 6. The crystal structures in FIG. 33A is adapted from Stadtmueller et al., Elife, Mar. 4, 2016, e10640 (SEQ ID NOS.: 250 and 253-257). FIG. 33B shows a cartoon representation of hpIgR domain-1 created from PDB ID 5D4K. CDR1, CDR2 and CDR3 of hpIgR domain-1 are shown in orange, pink and light red, respectively, wherein hpIgR domain-1 CDRs were swapped with corresponding teleost fish CDRs to test the influence of hpIgR domain-1 CDRs on VHH binding. FIG. 33C shows IgA binding to immobilized pIgR constructions, including K_{D} values (K_{D}, Kₒₙ, or K_{off}). FIG. 33D shows kinetic parameters for VHH2 and VHH3 binding to sensor immobilized HIS-tagged pIgR protein constructs. The K_{D}, Kₒₙ, or K_{off} are shown in the lower left, upper left and upper right panels, respectively. FIG. 33D shows that the hD1_tCDR2 construct did not show binding to both VHH2 and VHH3. Binding kinetic parameters were obtained by bio-layer interferometry, and the fold change in K_{D} values for VHH2 and VHH3 binding to pIgR domain constructs relative to full-length hpIgR ECD is shown in shown in the lower right panel.

FIG. 34 shows data describing how VHH2 and VHH3 compete with one another for binding to pIgR.

FIG. 35 illustrates that four molecules (VHH3, VHH4, VHH5 and VHH6) recognize buried epitopes on pIgR.

FIGS. 36A-36B shows that VHH3 recognizes a complex epitope on the hpIgR domain-1 interface.

FIGS. 37A-37B show results of VHH-mono-Fc molecules in forward and reverse transcytosis assays using MDCK-hpIgR monolayers, as described in Example 7. These results demonstrate bidirectional transport. FIG. 37A shows the results for the forward transcytosis (basolateral to apical direction), wherein 20 μg of test or control VHH-mono-Fc molecules were added to basolateral chamber and fold increase in apical [VHH] over control is shown at 24 hours (light gray) and 48 hours (dark gray) post treatment. For forward transcytosis, five VHH-mono-Fc molecules comprising a VHH2, VHH6, VHH9, VHH11 or VHH12 domain showed >20-fold increase in their apical concentration relative to control VHH-mono-Fc molecules at 48 hours, whereas VHH-mono-Fc molecules comprising a VHH4 domain showed a 15-fold increase in its apical concentration. FIG. 37B shows the results of reverse transcytosis (apical to basolateral direction), wherein 20 μg of test or control VHH-mono-Fc molecules were added to apical chamber and fold increase in basolateral [VHH] over control is shown at 24 hours (light gray) and 48 hours (dark gray) post treatment. VHH-mono-Fc molecules comprising a VHH6, VHH11, or VHH12 domain showed >10-fold increase in their basolateral concentration relative to control VHH-mono-Fc molecules at 48 hours. For reverse transcytosis, VHH-mono-Fc molecules comprising a VHH2, VHH4 or VHH9 domain showed >5-fold increase in their basolateral concentration relative to control VHH-mono-Fc molecules at 48 hours. Results for FIGS. 37A and 37B were obtained from three independent experiments, each containing two technical replicates.

FIGS. 38A-38B show results of VHH-mono-Fc molecules in forward and reverse transcytosis assays using MDCK-hpIgR monolayers, as described in Example 7. These resulted demonstrate bidirectional transport. To test forward transcytosis activity, 20 μg of test or control VHH-mono-Fc molecules were added to basolateral chamber and the amount of apical VHH-mono-Fc at 24 and 48 hours post treatment was quantified (B to A assay). To test reverse transcytosis activity, 20 μg of test or control VHH-mono-Fc molecules were added to apical chamber and the amount of basolateral VHH at 24 and 48 hours post treatment was quantified (A to B assay). Apical VHH (g) in B to A assay is shown in light gray and basolateral VHH (g) in A to B assay is shown in dark gray. FIG. 38A shows the comparison of forward and reverse transport of VHH-mono-Fc molecules at 24 hours post VHH treatment. FIG. 38B shows the comparison of forward and reverse transport of VHH-mono-Fc molecules at 48 hours post VHH treatment.

FIGS. 39A-39B show results for forward and reverse transcytosis kinetics of anti-pIgR VHH-mono-Fc molecules across MDCK-hpIgR monolayers, as described in Example 7. FIG. 39A shows the results of forward transcytosis kinetics (basolateral to apical direction), wherein 20 μg of test or control VHH-mono-Fc molecules were added to the basolateral chamber. The amount of VHH present in the apical chamber (g) was quantified and shown at different time points (0, 4, 8, 12, 24, 36 and 48 hours) post VHH treatment. The concentration of VHH-mono-Fc molecules increased over time in the apical chamber. For eight VHH-mono-Fc molecules, >10% of the basolateral VHH input (2 μg) was transported to the apical chamber (except VHH-mono-Fc molecules comprising a VHH3 or VHH7 domain). FIG. 39B shows the results of reverse transcytosis kinetics (apical to basolateral direction), wherein 20 μg of test or control VHH-mono-Fc molecules were added to the apical chamber. The amount of VHH present in the basolateral chamber (g) was quantified and shown at different time points (0, 4, 8, 12, 24, 36 and 48 hours) post VHH treatment. The concentration of VHH-mono-Fc molecules increased over time in the basolateral chamber. For six VHH-mono-Fc molecules, >10% of the apical VHH input (2 μg) was transported to the basolateral chamber (VHH-mono-Fc molecules comprising a VHH2, VHH4, VHH6, VHH9, VHH11 or VHH12 domain).

### 5. DETAILED DESCRIPTION

A description of example embodiments follows.

Described herein is the generation, screening and characterization of hpIgR-binding VHH molecules by biophysical and functional assays. VHH molecules showed varying degrees of affinity, species cross-reactivity, biophysical characteristics, epitope diversity, IgA competition profiles and transcytosis activity in a human lung tissue model. These VHH molecules may be useful as tools for studying the pIgR-mediated transport of biologics and as delivery vehicles for therapeutics. These VHH molecules may be useful for testing unexplored diagnostic and therapeutic applications in the pIgR space.

In one aspect is provided a VHH domain that binds to pIgR. In various embodiments, the VHH domain binds to an extracellular domain of pIgR, which can be, in some embodiments, extracellular domain 1 of pIgR, extracellular domain 2 of pIgR, extracellular domain 1-2 of pIgR, extracellular domain 3 of pIgR, extracellular domain 2-3 of pIgR, extracellular domain 4-5 of pIgR, or extracellular domain 5 of pIgR. Accordingly, in some embodiments, the VHH domain binds to extracellular domain 1 of pIgR. In some embodiments, the VHH domain binds to extracellular domain 2 of pIgR. In some embodiments, the VHH domain binds to extracellular domain 1-2 of pIgR. In some embodiments, the VHH domain binds to extracellular domain 3 of pIgR. In some embodiments, the VHH domain binds to extracellular domain 2-3 of pIgR. In some embodiments, the VHH domain binds to extracellular domain 4-5 of pIgR. In some embodiments, the VHH domain binds to extracellular domain 5 of pIgR. In some embodiments, the VHH domain binds to human pIgR and/or mouse pIgR.

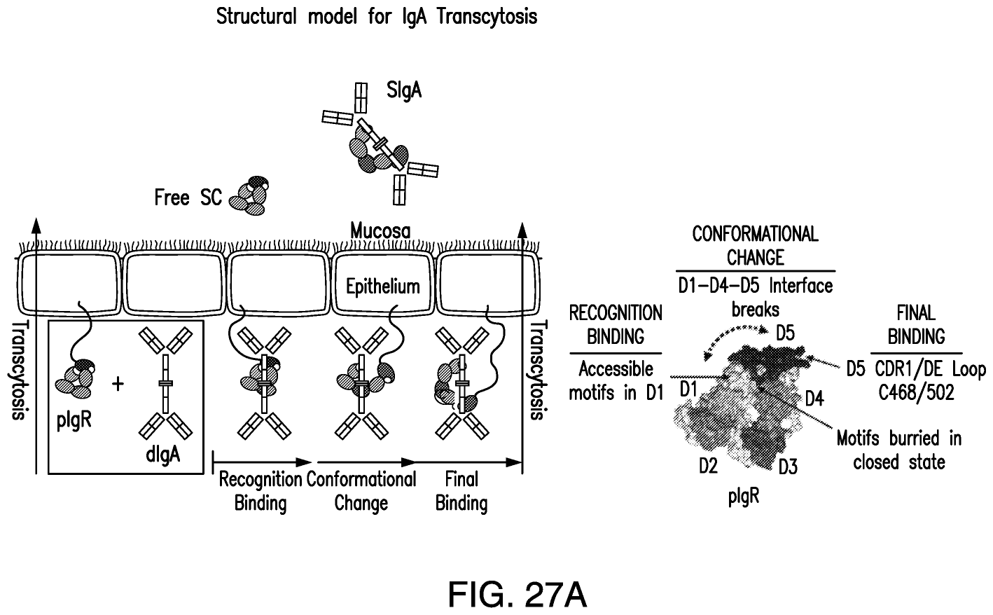

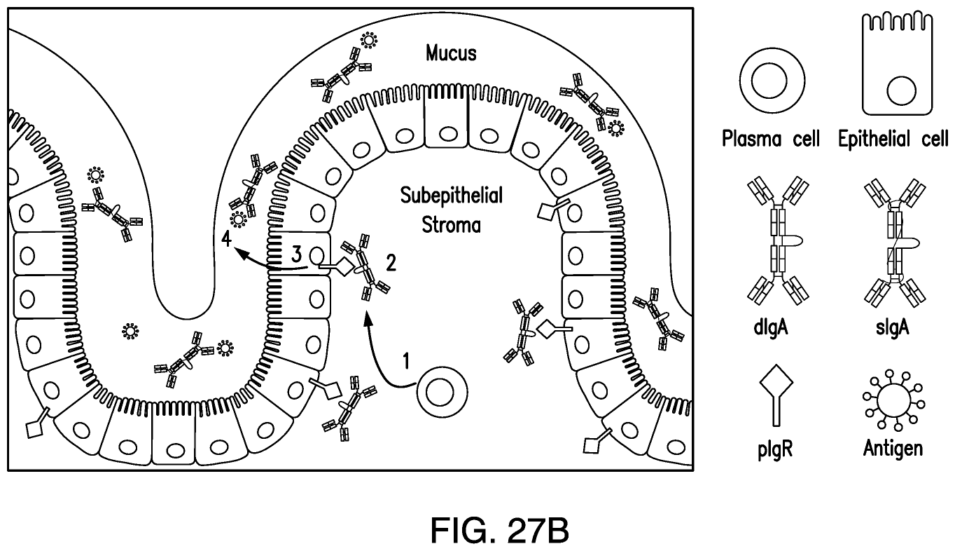

In various embodiments, the VHH domain is targeted to mucosal cells, even when the VHH domain is present in the bloodstream. Without wishing to be bound by theory, the pIgR is responsible for transcytosis of soluble polymeric IgA and IgM, but not IgG, into the mucosal lumen. A structural model for IgA transcytosis is shown in FIG. 27A and a schematic of pIgR-mediated transport of dIgA is shown in FIG. 27B. Though IgG molecules lack a lumen-targeted active transport mechanism, conferring pIgR-binding abilities to IgG can mediate selective transport of IgG antibodies into the mucosal lumen. Described herein are various anti-pIgR binding VHH domains that can act as trans-epithelial delivery moiety for the transport of any number of biotherapeutics. pIgR-binding VHH molecules may show varying degrees of affinity, species cross-reactivity, biophysical characteristics, epitope diversity, IgA competition profiles and transcytosis activity in a human lung tissue model.

Human pIgR (hpIgR) is an 82 kDa, single-pass transmembrane receptor containing a 620-residue extracellular domain (ECD), a 23-residue transmembrane domain and a 103-residue intracellular domain. pIgR transports soluble polymeric forms of IgA and IgM into apical mucosal tissues from the basolateral side of the epithelium. The process of transporting polymeric immunoglobulins from the basolateral to apical side is transcytosis. Following transcytosis, the pIgR ECD that contains five domains (secretory component) is proteolytically cleaved and released into mucus with or without IgA.

Without wishing to be bound by theory, targeted delivery of diagnostics and therapeutics can overcome several issues in drug delivery, such as systemic toxicity, circulation, cell barriers, bioavailability, targeted and controlled release, PK and clearance. Targeted delivery of molecules to highly compartmentalized organs by preferred routes of administration would be highly beneficial. The human mucosa lines about 400 m² of epithelial barriers in the gut, lungs, urogenital tract, and associated tissues. Mucosal protection is largely conferred through the function of pIgR, the oldest identifiable Fc receptor. Mucosal surface is more than 200 times of that covered by skin. The mucosa is constantly exposed to the external environment and pathogens such as bacteria, viruses, etc. Because mucosal surfaces are ideal portals of entry to most pathogens, mucosal immunity has evolved as a discrete system that performs highly regulated novel immunologic tasks. Mucosa associated lymphoid tissue must continually maintain a delicate balance between active immunity, oral tolerance, and suppression of immune responses (MacDonald, T. T. The mucosal immune system. Parasite Immunol 25, 235-246 (2003)).

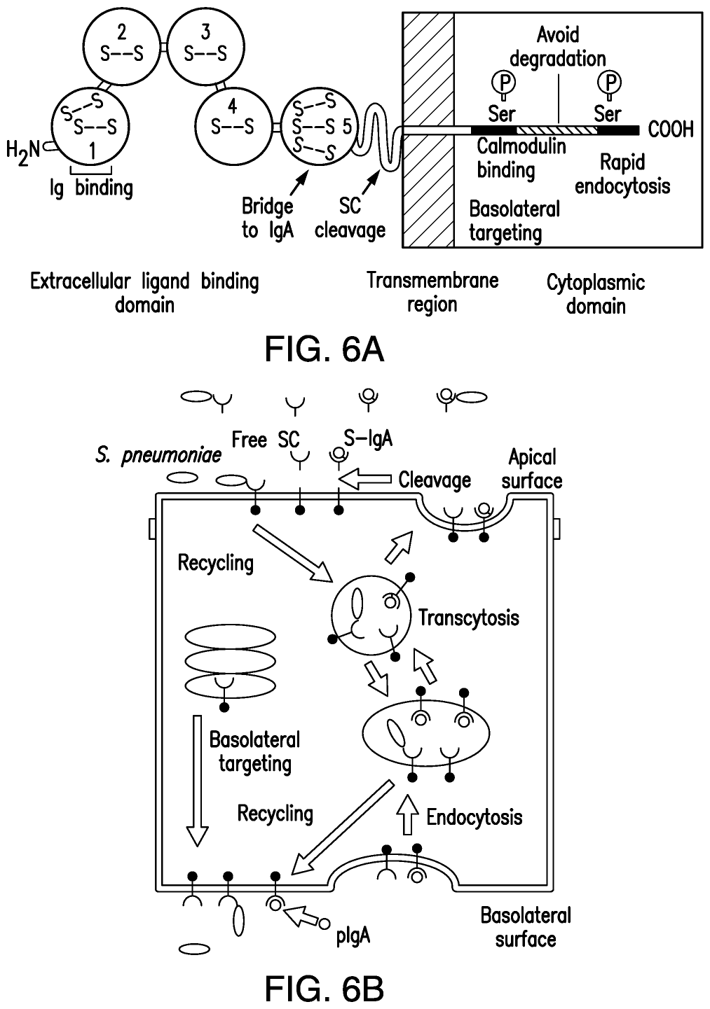

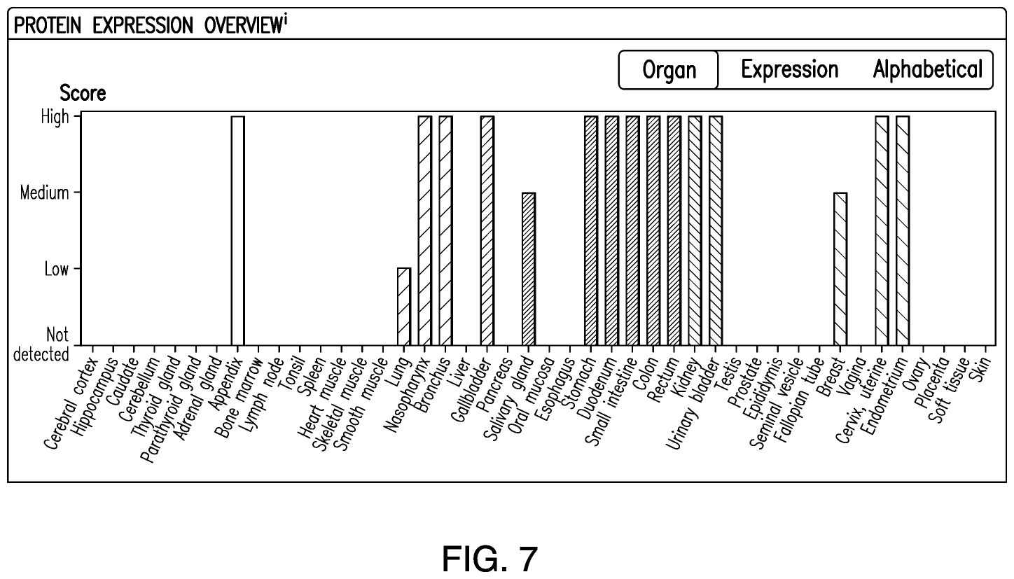

In various embodiments, the VHH molecules bind to human pIgR (Genbank ID: CR749533), a glycosylated type I membrane protein consisting of a 620-residue ectodomain with five tandem immunoglobulin-like domains, an extracellular C-terminus stalk, a 23-residue transmembrane domain, and a 103-residue intracellular domain (Turula, H. & Wobus, C. E. The Role of the Polymeric Immunoglobulin Receptor and Secretory Immunoglobulins during Mucosal Infection and Immunity. Viruses 10 (2018)). The structure of pIgR is summarized in FIG. 6A. pIgR can transport soluble polymeric forms of IgA and IgM into apical mucosal tissues from the basolateral side of the epithelium. A mechanism of pIgR-mediated transport is summarized in FIG. 6B. The expression of pIgR in various organs is shown in FIG. 7.

The process of transporting polymeric immunoglobulins from the basolateral to apical side is known as forward transcytosis. Following forward transcytosis, the five-domain containing ECD of pIgR (also known as the secretory component) is proteolytically cleaved and released into the mucus with or without IgA. In addition to transcytosis pIgR has several different functions that include, but are not limited to, conferring stability to IgA, immune exclusion, anti-inflammatory properties and homeostasis of commensals in the mucosal immune system. The process of recycling cleaved pIgR from the apical side to the basolateral to is known as reverse transcytosis. By using this mechanism, pathogens such as S. pneumoniae can breach the epithelial barrier and enter the systemic circulation.

Without wishing to be bound by theory, approximately 75% of total daily antibody production is directed to IgA molecules. In humans, there are two Ca genes encoding IgA subclass: IgA1 and IgA2 (IgA2m(1) and (2) allotypes). IgA1 has elongated hinge region lacking in IgA2, that contains several O-glycan sites and is susceptible to proteolytic cleavage. Endogenous IgA is present in various forms in a compartment-dependent manner. Monomeric IgA (mIgA) is the predominant form in serum (at a concentration of 1-3 mg/mL), primarily as IgA1 (about 90%) produced in bone marrow. Dimeric IgA (dIgA) is formed via S-S bridging of the C-terminal Fc tailpiece with J chain. dIgA is produced locally at target site of action and transported across mucosal surface into secretions of respiratory, GI and genitourinary tracts. Secretory IgA (S-IgA) is formed via dIgA complex with extracellular domain of polymeric Ig receptor (pIgR). Cleavage of secretory component (SC) at the mucosal surface of epithelial cells releases S-IgA.

The pIgR binds to soluble dimeric IgA via Fc and J-chain mediated interactions. pIgR does not bind or transport IgG molecules across mucosal epithelium. Though IgG molecules lack a lumen-targeted active transport mechanism, conferring pIgR-binding abilities to IgG can mediate selective transport of IgG antibodies into the mucosal lumen.

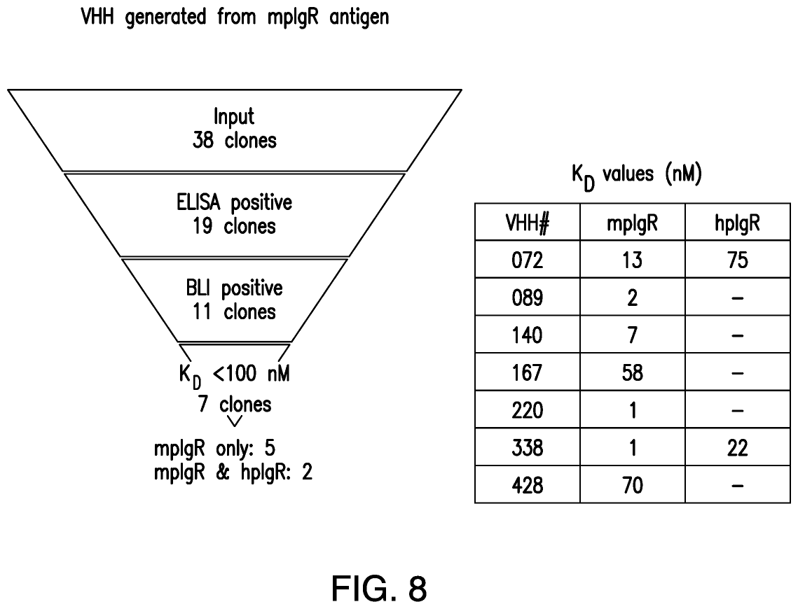

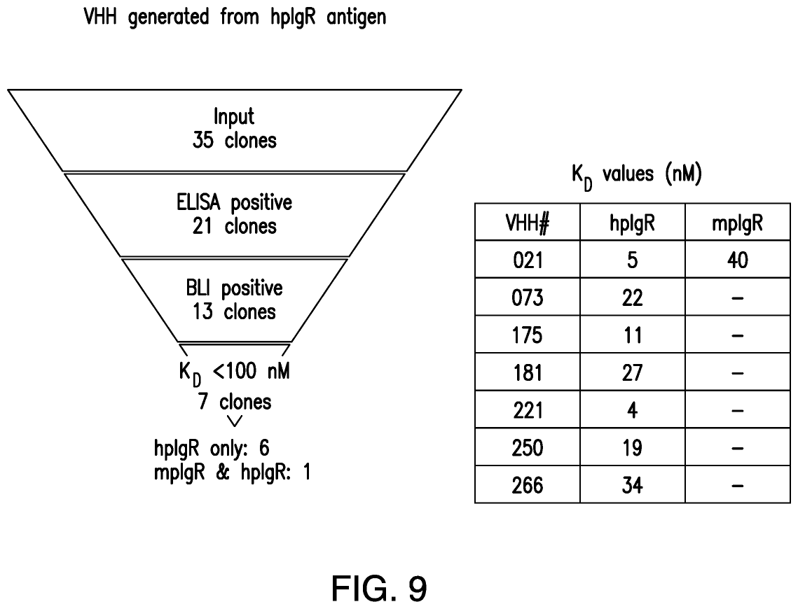

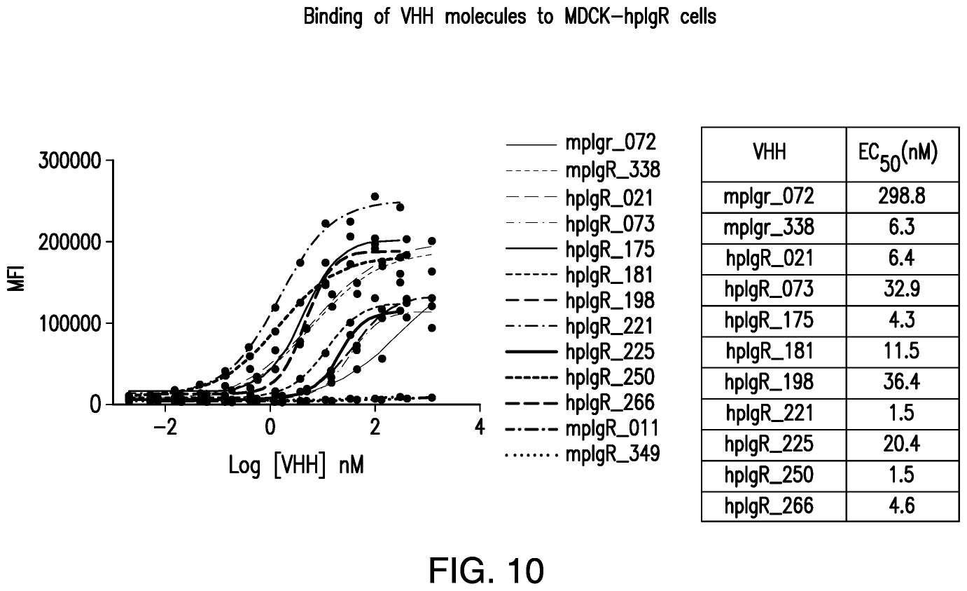

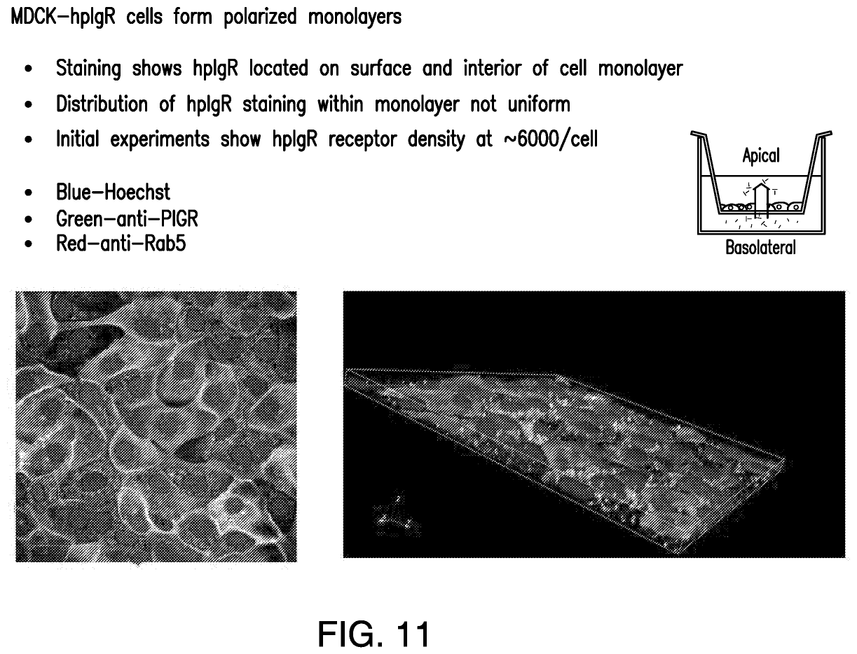

In various embodiments, the VHH domain is an anti-pIgR VHH sequence that can be genetically fused or chemically conjugated to any small-molecule or protein-based entity for delivery of these agents to pIgR-expressing cells, such as mucosal epithelial cells, and regions where pIgR ECD is present. VHH domains, or other pIgR binders, can be generated by immunizing llamas using mpIgR and hpIgR extracellular domain (ECD), performing single B-cell sorting, undertaking V-gene extraction, cloning the pIgR binders, such as VHH mono-Vc fusions, and then performing small scale expression and purification. Additional screening of the VHH binders and other molecules that bind to pIgR can be performed, including one or more of selecting for ELISA-positive, BLI-positive, and K_{D} less than 100 nM. These selection criteria can be combined as shown in FIG. 8 (VHH generated from mpIgR antigen) and FIG. 9 (VHH generated from hpIgR antigen). Additionally, individual VHH binders (and other molecules that bind to pIgR) can be assayed for their ability to bind to MDCK cells expressing pIgR, e.g., hpIgR. Such assay can be performed using FACS analysis with MDCK cells expressing hpIgR, and measuring the mean fluorescence intensity (MFI) of fluorescently-labeled VHH molecules. The results of such experiment are shown in FIG. 10. The staining of hpIgR on a monolayer of MDCK cells is shown in FIG. 11. The VHH domains of the disclosure may be generated in any animal that produces VHH-type antibodies, such as camelid family of animals such as llama or alpaca, or in animals, such as mouse, rat or chicken, engineered to express VHH molecules.

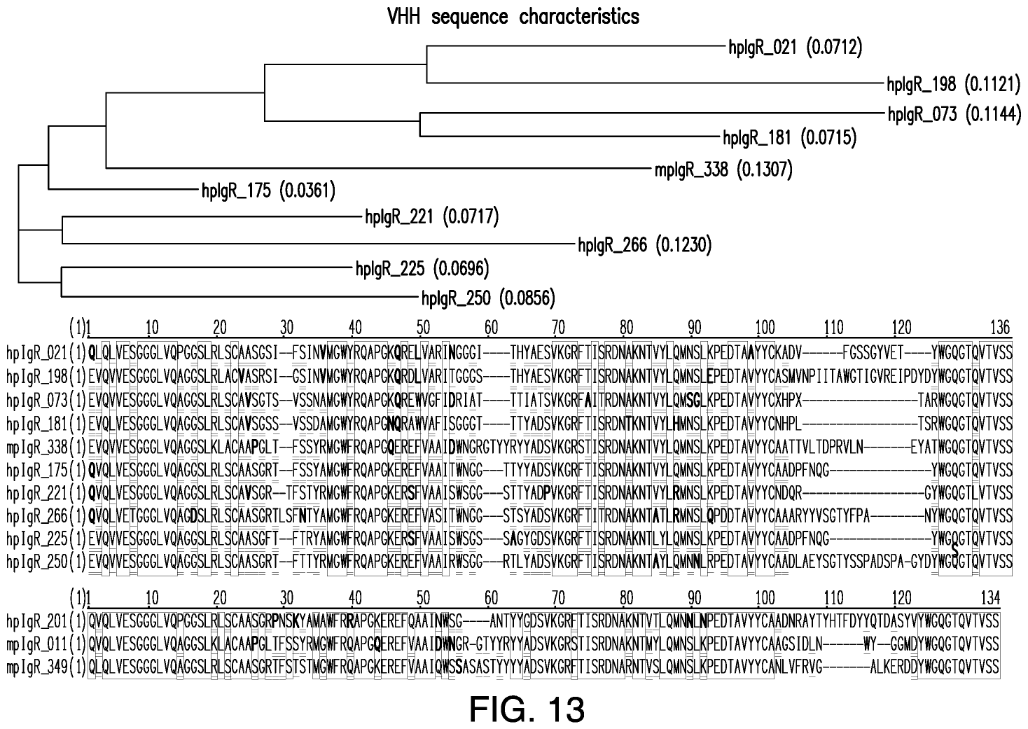

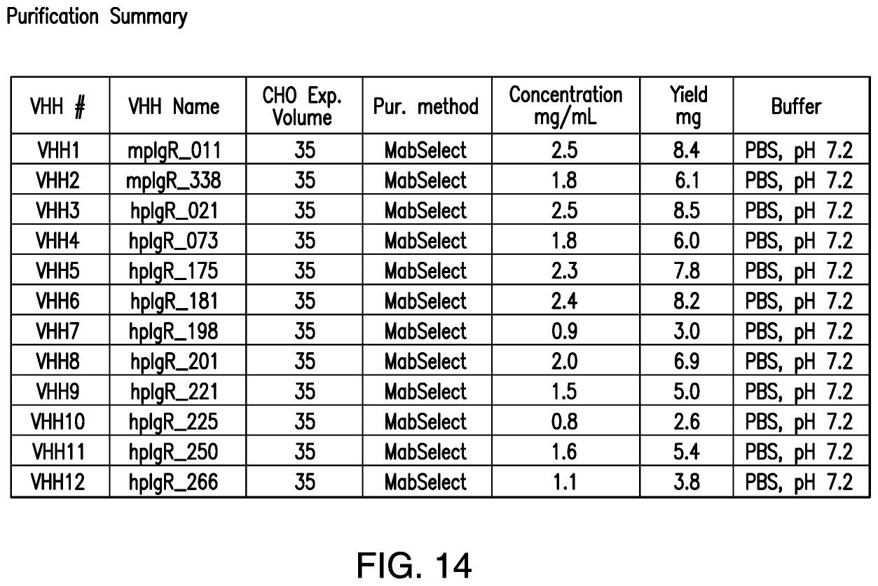

The set of VHH molecules (referred to as mpIgR_011, hpIgR_021, hpIgR_073, hpIgR_175, hpIgR_181, hpIgR_198, hpIgR_201, hpIgR_221, hpIgR_225, hpIgR_250, hpIgR_266, mpIgR_338, and hpIgR_349) do share some sequence characteristics, as shown in FIG. 13. Regions of highly conserved sequence similarity are shown in yellow. As indicated in FIG. 14, mpIgR_011 is VHH1, hpIgR_021 is VHH3, hpIgR_073 is VHH4, hpIgR_175 is VHH5, hpIgR_181 is VHH6, hpIgR_198 is VHH7, hpIgR_201 is VHH8, hpIgR_221 is VHH9, hpIgR_225 is VHH10, hpIgR_250 is VHH11, hpIgR_266 is VHH12, and mpIgR_338 is VHH2.

In some embodiments, the VHH domains as described herein bind to pIgR, but do not bind to the extracellular C-terminus stalk of pIgR. Accordingly, in some embodiments, the VHH domains described herein bind to an extracellular domain of pIgR, but do not bind to the amino acid sequence of human pIgR EKAVADTRDQADGSRASVDSGSSEEQGGSSR (SEQ ID NO: 143) and/or mouse pIgR EREIQNVGDQAQENRASGDAGSADGQSRSSSSK (SEQ ID NO: 144) or EREIQNVRDQAQENRASGDAGSADGQSRSSSSK (SEQ ID NO: 145).

Various VHH domains are described herein in Table 1. In some embodiments, the VHH domain competes with IgA binding to the pIgR. In some embodiments, the VHH domain promotes IgA binding to the pIgR. In some embodiments, the K_{D} of the binding of the VHH domain to pIgR is from about 4 to about 525 nM. In some embodiments, in the VHH domain, the K_{D} of the binding of the VHH domain to pIgR is less than about 50 nM. In some embodiments, the K_{D} of the binding of the VHH domain to pIgR is from about 4 to about 525 nM. In some embodiments, the K_{D} of the binding of the VHH domain to pIgR is from about 4 to about 34 nM. Bio-layer interferometry experiments described herein show 8 VHH domain binders having K_{D} values of <50 nM for binding to the human pIgR ectodomain (Table 1). In some embodiments, the Tₘ of the VHH domain is from about 53 to about 77° C. In some embodiments, the Tₘ of the VHH domain is from 53.9 to 76.4° C. In some embodiments, the Tₘ of the VHH domain is from about 61 to about 77° C. In some embodiments, the Tₘ of the VHH domain is from about 61 to about 71° C. In some embodiments, the EC50 value for VHH domain binding to an MDCK-hpIgR cell is less than about 10 nM. Six such binders comprising a VHH domain are described in Table 1. Competition with IgA for binding to pIgR, K_{D}, and Tₘ of the VHH domain of the disclosure may be evaluated using methods described herein.

In a general aspect is provided is a set of anti-pIgR VHH sequences that can be genetically fused or chemically conjugated to any small-molecule or protein-based entities for delivery of desired molecules into or across pIgR-expressing cells such as mucosal epithelial cells.

In another general aspect is provided is a set of anti-pIgR VHH sequences that can be genetically fused or chemically conjugated to any small-molecule or protein-based entities for modulating the biochemical, biophysical, cell biological and pharmacological parameters of desired fusion molecules.

In various embodiments of the aspects described herein the VHH domain comprises a CDR1 sequence present in VHH1, e.g., the CDR1 sequence of SYRMG (SEQ ID NO: 1). In various embodiments of the aspects described herein the VHH domain comprises a CDR1 sequence present in VHH2, e.g., the CDR1 sequence of SYRMG (SEQ ID NO: 1). In various embodiments of the aspects described herein the VHH domain comprises a CDR1 sequence present in VHH3, e.g., the CDR1 sequence of INVMG (SEQ ID NO: 2). In various embodiments of the aspects described herein the VHH domain comprises a CDR1 sequence present in VHH4, e.g., the CDR1 sequence of SNAMG (SEQ ID NO: 3). In various embodiments of the aspects described herein the VHH domain comprises a CDR1 sequence present in VHH5, e.g., the CDR1 sequence of SYAMG (SEQ ID NO: 4). In various embodiments of the aspects described herein the VHH domain comprises a CDR1 sequence present in VHH6, e.g., the CDR1 sequence of SDAMG (SEQ ID NO: 5). In various embodiments of the aspects described herein the VHH domain comprises a CDR1 sequence present in VHH7, e.g., the CDR1 sequence of INVMG (SEQ ID NO: 6). In various embodiments of the aspects described herein the VHH domain comprises a CDR1 sequence present in VHH9, e.g., the CDR1 sequence of TYRMG (SEQ ID NO: 7). In various embodiments of the aspects described herein the VHH domain comprises a CDR1 sequence present in VHH10, e.g., the CDR1 sequence of RYAMG (SEQ ID NO: 8). In various embodiments of the aspects described herein the VHH domain comprises a CDR1 sequence present in VHH11, e.g., the CDR1 sequence of TYRMG (SEQ ID NO: 259). In various embodiments of the aspects described herein the VHH domain comprises a CDR1 sequence present in VHH12, e.g., the CDR1 sequence of FNTYAMG (SEQ ID NO: 9).

In various embodiments of the aspects described herein the VHH domain comprises a CDR1 sequence present in VHH1, e.g., the CDR1 sequence of GLTFSSY (SEQ ID NO: 10). In various embodiments of the aspects described herein the VHH domain comprises a CDR1 sequence present in VHH2, e.g., the CDR1 sequence of GLTFSSY (SEQ ID NO: 10). In various embodiments of the aspects described herein the VHH domain comprises a CDR1 sequence present in VHH3, e.g., the CDR1 sequence of GSIFSIN (SEQ ID NO: 11). In various embodiments of the aspects described herein the VHH domain comprises a CDR1 sequence present in VHH4, e.g., the CDR1 sequence of GTSVSSN (SEQ ID NO: 12). In various embodiments of the aspects described herein the VHH domain comprises a CDR1 sequence present in VHH5, e.g., the CDR1 sequence of GRTFSSY (SEQ ID NO: 13). In various embodiments of the aspects described herein the VHH domain comprises a CDR1 sequence present in VHH6, e.g., the CDR1 sequence of GSSVSSD (SEQ ID NO: 14). In various embodiments of the aspects described herein the VHH domain comprises a CDR1 sequence present in VHH7, e.g., the CDR1 sequence of RSIGSIN (SEQ ID NO: 15). In various embodiments of the aspects described herein the VHH domain comprises a CDR1 sequence present in VHH9, e.g., the CDR1 sequence of GRTFSTY (SEQ ID NO: 16). In various embodiments of the aspects described herein the VHH domain comprises a CDR1 sequence present in VHH10, e.g., the CDR1 sequence of GFTFTRY (SEQ ID NO: 17). In various embodiments of the aspects described herein the VHH domain comprises a CDR1 sequence present in VHH11, e.g., the CDR1 sequence of GRTFTTY (SEQ ID NO: 18). In various embodiments of the aspects described herein the VHH domain comprises a CDR1 sequence present in VHH12, e.g., the CDR1 sequence of GRTLSFNTY (SEQ ID NO: 19).

In various embodiments of the aspects described herein the VHH domain comprises a CDR1 sequence present in VHH1, e.g., the CDR1 sequence of GLTFSSYR (SEQ ID NO: 20). In various embodiments of the aspects described herein the VHH domain comprises a CDR1 sequence present in VHH2, e.g., the CDR1 sequence of GLTFSSYR (SEQ ID NO: 20). In various embodiments of the aspects described herein the VHH domain comprises a CDR1 sequence present in VHH3, e.g., the CDR1 sequence of GSIFSINV (SEQ ID NO: 21). In various embodiments of the aspects described herein the VHH domain comprises a CDR1 sequence present in VHH4, e.g., the CDR1 sequence of GTSVSSNA (SEQ ID NO: 22). In various embodiments of the aspects described herein the VHH domain comprises a CDR1 sequence present in VHH5, e.g., the CDR1 sequence of GRTFSSYA (SEQ ID NO: 23). In various embodiments of the aspects described herein the VHH domain comprises a CDR1 sequence present in VHH6, e.g., the CDR1 sequence of GSSVSSDA (SEQ ID NO: 24). In various embodiments of the aspects described herein the VHH domain comprises a CDR1 sequence present in VHH7, e.g., the CDR1 sequence of RSIGSINV (SEQ ID NO: 25). In various embodiments of the aspects described herein the VHH domain comprises a CDR1 sequence present in VHH9, e.g., the CDR1 sequence of GRTFSTYR (SEQ ID NO: 26). In various embodiments of the aspects described herein the VHH domain comprises a CDR1 sequence present in VHH10, e.g., the CDR1 sequence of GFTFTRYA (SEQ ID NO: 27). In various embodiments of the aspects described herein the VHH domain comprises a CDR1 sequence present in VHH11, e.g., the CDR1 sequence of GRTFTTYR (SEQ ID NO: 28). In various embodiments of the aspects described herein the VHH domain comprises a CDR1 sequence present in VHH12, e.g., the CDR1 sequence of GRTLSFNTYA (SEQ ID NO: 29).

In various embodiments of the aspects described herein the VHH domain comprises a CDR1 sequence present in VHH1, e.g., the CDR1 sequence of GLTFSSYRMG (SEQ ID NO: 154). In various embodiments of the aspects described herein the VHH domain comprises a CDR1 sequence present in VHH2, e.g., the CDR1 sequence of GLTFSSYRMG (SEQ ID NO: 154). In various embodiments of the aspects described herein the VHH domain comprises a CDR1 sequence present in VHH3, e.g., the CDR1 sequence of GSIFSINVMG (SEQ ID NO: 155). In various embodiments of the aspects described herein the VHH domain comprises a CDR1 sequence present in VHH4, e.g., the CDR1 sequence of GTSVSSNAMG (SEQ ID NO: 156). In various embodiments of the aspects described herein the VHH domain comprises a CDR1 sequence present in VHH5, e.g., the CDR1 sequence of GRTFSSYAMG (SEQ ID NO: 157). In various embodiments of the aspects described herein the VHH domain comprises a CDR1 sequence present in VHH6, e.g., the CDR1 sequence of GSSVSSDAMG (SEQ ID NO: 158). In various embodiments of the aspects described herein the VHH domain comprises a CDR1 sequence present in VHH7, e.g., the CDR1 sequence of RSIGSINVMG (SEQ ID NO: 159). In various embodiments of the aspects described herein the VHH domain comprises a CDR1 sequence present in VHH9, e.g., the CDR1 sequence of GRTFSTYRMG (SEQ ID NO: 160). In various embodiments of the aspects described herein the VHH domain comprises a CDR1 sequence present in VHH10, e.g., the CDR1 sequence of RYAMG GFTFTRYAMG (SEQ ID NO: 161). In various embodiments of the aspects described herein the VHH domain comprises a CDR1 sequence present in VHH11, e.g., the CDR1 sequence of GRTFTTYRMG (SEQ ID NO: 162). In various embodiments of the aspects described herein the VHH domain comprises a CDR1 sequence present in VHH12, e.g., the CDR1 sequence of GRTLSFNTYAMG (SEQ ID NO: 163).

In various embodiments of the aspects described herein the VHH domain comprises a CDR1 sequence present in VHH1, e.g., the CDR1 sequence of GLTFSSY SSYRMG (SEQ ID NO: 164). In various embodiments of the aspects described herein the VHH domain comprises a CDR1 sequence present in VHH2, e.g., the CDR1 sequence of SSYRMG (SEQ ID NO: 164). In various embodiments of the aspects described herein the VHH domain comprises a CDR1 sequence present in VHH3, e.g., the CDR1 sequence of SINVMG (SEQ ID NO: 165). In various embodiments of the aspects described herein the VHH domain comprises a CDR1 sequence present in VHH4, e.g., the CDR1 sequence of SSNAMG (SEQ ID NO: 166). In various embodiments of the aspects described herein the VHH domain comprises a CDR1 sequence present in VHH5, e.g., the CDR1 sequence of SSYAMG (SEQ ID NO: 167). In various embodiments of the aspects described herein the VHH domain comprises a CDR1 sequence present in VHH6, e.g., the CDR1 sequence of SSDAMG (SEQ ID NO: 168). In various embodiments of the aspects described herein the VHH domain comprises a CDR1 sequence present in VHH7, e.g., the CDR1 sequence of SINVMG (SEQ ID NO: 169). In various embodiments of the aspects described herein the VHH domain comprises a CDR1 sequence present in VHH9, e.g., the CDR1 sequence of STYRMG (SEQ ID NO: 170). In various embodiments of the aspects described herein the VHH domain comprises a CDR1 sequence present in VHH10, e.g., the CDR1 sequence of TRYAMG (SEQ ID NO: 171). In various embodiments of the aspects described herein the VHH domain comprises a CDR1 sequence present in VHH11, e.g., the CDR1 sequence of TTYRMG (SEQ ID NO: 172). In various embodiments of the aspects described herein the VHH domain comprises a CDR1 sequence present in VHH12, e.g., the CDR1 sequence of SFNTYAMG (SEQ ID NO: 173).

In various embodiments of the aspects described herein the VHH domain comprises a CDR1 sequence present in VHH1, e.g., the CDR1 sequence of GLTFSSYRMG (SEQ ID NO: 174). In various embodiments of the aspects described herein the VHH domain comprises a CDR1 sequence present in VHH2, e.g., the CDR1 sequence of GLTFSSYRMG (SEQ ID NO: 174). In various embodiments of the aspects described herein the VHH domain comprises a CDR1 sequence present in VHH3, e.g., the CDR1 sequence of GSIFSINVMG (SEQ ID NO: 175). In various embodiments of the aspects described herein the VHH domain comprises a CDR1 sequence present in VHH4, e.g., the CDR1 sequence of GTSVSSNAMG (SEQ ID NO: 176). In various embodiments of the aspects described herein the VHH domain comprises a CDR1 sequence present in VHH5, e.g., the CDR1 sequence of GRTFSSYAMG (SEQ ID NO: 177). In various embodiments of the aspects described herein the VHH domain comprises a CDR1 sequence present in VHH6, e.g., the CDR1 sequence of GSSVSSDAMG (SEQ ID NO: 178). In various embodiments of the aspects described herein the VHH domain comprises a CDR1 sequence present in VHH7, e.g., the CDR1 sequence of RSIGSINVMG (SEQ ID NO: 179). In various embodiments of the aspects described herein the VHH domain comprises a CDR1 sequence present in VHH9, e.g., the CDR1 sequence of GRTFSTYRMG (SEQ ID NO: 180). In various embodiments of the aspects described herein the VHH domain comprises a CDR1 sequence present in VHH10, e.g., the CDR1 sequence of GFTFTRYAMG (SEQ ID NO: 181). In various embodiments of the aspects described herein the VHH domain comprises a CDR1 sequence present in VHH11, e.g., the CDR1 sequence of GRTFTTYRMG (SEQ ID NO: 182). In various embodiments of the aspects described herein the VHH domain comprises a CDR1 sequence present in VHH12, e.g., the CDR1 sequence of GRTLSFNTYAMG (SEQ ID NO: 183).

In various embodiments of the aspects described herein the VHH domain comprises a CDR2 sequence present in VHH1, e.g., the CDR2 sequence of AIDWNGRGTYYRYYADSVKG (SEQ ID NO: 30). In various embodiments of the aspects described herein the VHH domain comprises a CDR2 sequence present in VHH2, e.g., the CDR2 sequence of AIDWNGRGTYYRYYADSVKG (SEQ ID NO: 30). In various embodiments of the aspects described herein the VHH domain comprises a CDR2 sequence present in VHH3, e.g., the CDR2 sequence of RINGGGITHYAESVKG (SEQ ID NO: 31). In various embodiments of the aspects described herein the VHH domain comprises a CDR2 sequence present in VHH4, e.g., the CDR2 sequence of FIDRIATTTIATSVKG (SEQ ID NO: 32). In various embodiments of the aspects described herein the VHH domain comprises a CDR2 sequence present in VHH5, e.g., the CDR2 sequence of AITWNGGTTYYADSVKG (SEQ ID NO: 33). In various embodiments of the aspects described herein the VHH domain comprises a CDR2 sequence present in VHH6, e.g., the CDR2 sequence of FISGGGTTTYADSVKG (SEQ ID NO: 34). In various embodiments of the aspects described herein the VHH domain comprises a CDR2 sequence present in VHH7, e.g., the CDR2 sequence of RITGGGSTHYAESVKG (SEQ ID NO: 35). In various embodiments of the aspects described herein the VHH domain comprises a CDR2 sequence present in VHH9, e.g., the CDR2 sequence of AISWSGGSTTYADPVKG (SEQ ID NO: 36). In various embodiments of the aspects described herein the VHH domain comprises a CDR2 sequence present in VHH10, e.g., the CDR2 sequence of AISWSGSSAGYGDSVKG (SEQ ID NO: 37). In various embodiments of the aspects described herein the VHH domain comprises a CDR2 sequence present in VHH11, e.g., the CDR2 sequence of AIRWSGGRTLYADSVKG (SEQ ID NO: 38). In various embodiments of the aspects described herein the VHH domain comprises a CDR2 sequence present in VHH12, e.g., the CDR2 sequence of SITWNGGSTSYADSVKG (SEQ ID NO: 39).

In various embodiments of the aspects described herein the VHH domain comprises a CDR2 sequence present in VHH1, e.g., the CDR2 sequence of DWNGRGTYY (SEQ ID NO: 40) or WNGRGTY (SEQ ID NO: 260). In various embodiments of the aspects described herein the VHH domain comprises a CDR2 sequence present in VHH2, e.g., the CDR2 sequence of DWNGRGTYY (SEQ ID NO: 40) or WNGRGTY (SEQ ID NO: 260). In various embodiments of the aspects described herein the VHH domain comprises a CDR2 sequence present in VHH3, e.g., the CDR2 sequence of NGGGI (SEQ ID NO: 41) or GGG (SEQ ID NO: 261). In various embodiments of the aspects described herein the VHH domain comprises a CDR2 sequence present in VHH4, e.g., the CDR2 sequence of DRIAT (SEQ ID NO: 42) or RIA (SEQ ID NO: 262). In various embodiments of the aspects described herein the VHH domain comprises a CDR2 sequence present in VHH5, e.g., the CDR2 sequence of TWNGGT (SEQ ID NO: 43) or WNGG (SEQ ID NO: 263). In various embodiments of the aspects described herein the VHH domain comprises a CDR2 sequence present in VHH6, e.g., the CDR2 sequence of SGGGT (SEQ ID NO: 44) or GGG (SEQ ID NO: 264). In various embodiments of the aspects described herein the VHH domain comprises a CDR2 sequence present in VHH7, e.g., the CDR2 sequence of TGGGS (SEQ ID NO: 45) or GGG (SEQ ID NO: 265). In various embodiments of the aspects described herein the VHH domain comprises a CDR2 sequence present in VHH9, e.g., the CDR2 sequence of SWSGGS (SEQ ID NO: 46) or WSGG (SEQ ID NO: 266). In various embodiments of the aspects described herein the VHH domain comprises a CDR2 sequence present in VHH10, e.g., the CDR2 sequence of SWSGGS (SEQ ID NO: 47) or WSGS (SEQ ID NO: 267). In various embodiments of the aspects described herein the VHH domain comprises a CDR2 sequence present in VHH11, e.g., the CDR2 sequence of RWSGGR (SEQ ID NO: 48) or WSGG (SEQ ID NO: 268). In various embodiments of the aspects described herein the VHH domain comprises a CDR2 sequence present in VHH12, e.g., the CDR2 sequence of TWNGGS (SEQ ID NO: 49) or WNGG (SEQ ID NO: 269).

In various embodiments of the aspects described herein the VHH domain comprises a CDR2 sequence present in VHH1, e.g., the CDR2 sequence of IDWNGRGTYY (SEQ ID NO: 50) or IDWNGRGTYYR (SEQ ID NO: 270). In various embodiments of the aspects described herein the VHH domain comprises a CDR2 sequence present in VHH2, e.g., the CDR2 sequence of IDWNGRGTYY (SEQ ID NO: 50) or IDWNGRGTYYR (SEQ ID NO: 270). In various embodiments of the aspects described herein the VHH domain comprises a CDR2 sequence present in VHH3, e.g., the CDR2 sequence of INGGGIT (SEQ ID NO: 51). In various embodiments of the aspects described herein the VHH domain comprises a CDR2 sequence present in VHH4, e.g., the CDR2 sequence of IDRIATT (SEQ ID NO: 52). In various embodiments of the aspects described herein the VHH domain comprises a CDR2 sequence present in VHH5, e.g., the CDR2 sequence of ITWNGGTT (SEQ ID NO: 53). In various embodiments of the aspects described herein the VHH domain comprises a CDR2 sequence present in VHH6, e.g., the CDR2 sequence of ISGGGTT (SEQ ID NO: 54). In various embodiments of the aspects described herein the VHH domain comprises a CDR2 sequence present in VHH7, e.g., the CDR2 sequence of ITGGGST (SEQ ID NO: 55). In various embodiments of the aspects described herein the VHH domain comprises a CDR2 sequence present in VHH9, e.g., the CDR2 sequence of ISWSGGST (SEQ ID NO: 56). In various embodiments of the aspects described herein the VHH domain comprises a CDR2 sequence present in VHH10, e.g., the CDR2 sequence of ISWSGSSA (SEQ ID NO: 57). In various embodiments of the aspects described herein the VHH domain comprises a CDR2 sequence present in VHH11, e.g., the CDR2 sequence of IRWSGGRT (SEQ ID NO: 58). In various embodiments of the aspects described herein the VHH domain comprises a CDR2 sequence present in VHH12, e.g., the CDR2 sequence of ITWNGGST (SEQ ID NO: 59).

In various embodiments of the aspects described herein the VHH domain comprises a CDR2 sequence present in VHH1, e.g., the CDR2 sequence of AIDWNGRGTYYRYYADSVKG (SEQ ID NO: 184). In various embodiments of the aspects described herein the VHH domain comprises a CDR2 sequence present in VHH2, e.g., the CDR2 sequence of AIDWNGRGTYYRYYADSVKG (SEQ ID NO: 184). In various embodiments of the aspects described herein the VHH domain comprises a CDR2 sequence present in VHH3, e.g., the CDR2 sequence of RINGGGITHYAESVKG (SEQ ID NO: 185). In various embodiments of the aspects described herein the VHH domain comprises a CDR2 sequence present in VHH4, e.g., the CDR2 sequence of FIDRIATTTIATSVKG (SEQ ID NO: 186). In various embodiments of the aspects described herein the VHH domain comprises a CDR2 sequence present in VHH5, e.g., the CDR2 sequence of AITWNGGTTYYADSVKG (SEQ ID NO: 187). In various embodiments of the aspects described herein the VHH domain comprises a CDR2 sequence present in VHH6, e.g., the CDR2 sequence of FISGGGTTTYADSVKG (SEQ ID NO: 188). In various embodiments of the aspects described herein the VHH domain comprises a CDR2 sequence present in VHH7, e.g., the CDR2 sequence of RITGGGSTHYAESVKG (SEQ ID NO: 189). In various embodiments of the aspects described herein the VHH domain comprises a CDR2 sequence present in VHH9, e.g., the CDR2 sequence of AISWSGGSTTYADPVKG (SEQ ID NO: 190). In various embodiments of the aspects described herein the VHH domain comprises a CDR2 sequence present in VHH10, e.g., the CDR2 sequence of AISWSGSSAGYGDSVKG (SEQ ID NO: 191). In various embodiments of the aspects described herein the VHH domain comprises a CDR2 sequence present in VHH11, e.g., the CDR2 sequence of AIRWSGGRTLYADSVKG (SEQ ID NO: 192). In various embodiments of the aspects described herein the VHH domain comprises a CDR2 sequence present in VHH12, e.g., the CDR2 sequence of SITWNGGSTSYADSVKG (SEQ ID NO: 193).

In various embodiments of the aspects described herein the VHH domain comprises a CDR2 sequence present in VHH1, e.g., the CDR2 sequence of FVAAIDWNGRGTYYRY (SEQ ID NO: 194). In various embodiments of the aspects described herein the VHH domain comprises a CDR2 sequence present in VHH2, e.g., the CDR2 sequence of FVAAIDWNGRGTYYRY (SEQ ID NO: 194). In various embodiments of the aspects described herein the VHH domain comprises a CDR2 sequence present in VHH3, e.g., the CDR2 sequence of LVARINGGGITH (SEQ ID NO: 195). In various embodiments of the aspects described herein the VHH domain comprises a CDR2 sequence present in VHH4, e.g., the CDR2 sequence of WVGFIDRIATTT (SEQ ID NO: 196). In various embodiments of the aspects described herein the VHH domain comprises a CDR2 sequence present in VHH5, e.g., the CDR2 sequence of FVAAITWNGGTTY (SEQ ID NO: 197). In various embodiments of the aspects described herein the VHH domain comprises a CDR2 sequence present in VHH6, e.g., the CDR2 sequence of WVAFISGGGTTT (SEQ ID NO: 198). In various embodiments of the aspects described herein the VHH domain comprises a CDR2 sequence present in VHH7, e.g., the CDR2 sequence of LVARITGGGSTH (SEQ ID NO: 199). In various embodiments of the aspects described herein the VHH domain comprises a CDR2 sequence present in VHH9, e.g., the CDR2 sequence of FVAAISWSGGSTT (SEQ ID NO: 200). In various embodiments of the aspects described herein the VHH domain comprises a CDR2 sequence present in VHH10, e.g., the CDR2 sequence of FVAAISWSGSSAG (SEQ ID NO: 201). In various embodiments of the aspects described herein the VHH domain comprises a CDR2 sequence present in VHH11, e.g., the CDR2 sequence of FVAAIRWSGGRTL (SEQ ID NO: 202). In various embodiments of the aspects described herein the VHH domain comprises a CDR2 sequence present in VHH12, e.g., the CDR2 sequence of FVASITWNGGSTS (SEQ ID NO: 203).

In various embodiments of the aspects described herein the VHH domain comprises a CDR2 sequence present in VHH1, e.g., the CDR2 sequence of AIDWNGRGTYYRY (SEQ ID NO: 204). In various embodiments of the aspects described herein the VHH domain comprises a CDR2 sequence present in VHH2, e.g., the CDR2 sequence of AIDWNGRGTYYRY (SEQ ID NO: 204). In various embodiments of the aspects described herein the VHH domain comprises a CDR2 sequence present in VHH3, e.g., the CDR2 sequence of RINGGGITH (SEQ ID NO: 205). In various embodiments of the aspects described herein the VHH domain comprises a CDR2 sequence present in VHH4, e.g., the CDR2 sequence of FIDRIATTT (SEQ ID NO: 206). In various embodiments of the aspects described herein the VHH domain comprises a CDR2 sequence present in VHH5, e.g., the CDR2 sequence of AITWNGGTTY (SEQ ID NO: 207). In various embodiments of the aspects described herein the VHH domain comprises a CDR2 sequence present in VHH6, e.g., the CDR2 sequence of FISGGGTTT (SEQ ID NO: 208). In various embodiments of the aspects described herein the VHH domain comprises a CDR2 sequence present in VHH7, e.g., the CDR2 sequence of RITGGGSTH (SEQ ID NO: 209). In various embodiments of the aspects described herein the VHH domain comprises a CDR2 sequence present in VHH9, e.g., the CDR2 sequence of AISWSGGSTT (SEQ ID NO: 210). In various embodiments of the aspects described herein the VHH domain comprises a CDR2 sequence present in VHH10, e.g., the CDR2 sequence of AISWSGSSAG (SEQ ID NO: 211). In various embodiments of the aspects described herein the VHH domain comprises a CDR2 sequence present in VHH11, e.g., the CDR2 sequence of AIRWSGGRTL (SEQ ID NO: 212). In various embodiments of the aspects described herein the VHH domain comprises a CDR2 sequence present in VHH12, e.g., the CDR2 sequence of SITWNGGSTS (SEQ ID NO: 213).

In various embodiments of the aspects described herein the VHH domain comprises a CDR3 sequence present in VHH1, e.g., the CDR3 sequence of GSIDLNWYGGMDY (SEQ ID NO: 60). In various embodiments of the aspects described herein the VHH domain comprises a CDR3 sequence present in VHH2, e.g., the CDR3 sequence of TTVLTDPRVLNEYAT (SEQ ID NO: 61). In various embodiments of the aspects described herein the VHH domain comprises a CDR3 sequence present in VHH3, e.g., the CDR3 sequence of DVFGSSGYVETY (SEQ ID NO: 62). In various embodiments of the aspects described herein the VHH domain comprises a CDR3 sequence present in VHH4, e.g., the CDR3 sequence of PLTAR (SEQ ID NO: 63). In various embodiments of the aspects described herein the VHH domain comprises a CDR3 sequence present in VHH5, e.g., the CDR3 sequence of DPFNQGY (SEQ ID NO: 64). In various embodiments of the aspects described herein the VHH domain comprises a CDR3 sequence present in VHH6, e.g., the CDR3 sequence of PLTSR (SEQ ID NO: 65). In various embodiments of the aspects described herein the VHH domain comprises a CDR3 sequence present in VHH7, e.g., the CDR3 sequence of MVNPIITAWGTIGVREIPDYDY (SEQ ID NO: 66). In various embodiments of the aspects described herein the VHH domain comprises a CDR3 sequence present in VHH9, e.g., the CDR3 sequence of DQRGY (SEQ ID NO: 67) or QRGY (SEQ ID NO: 271). In various embodiments of the aspects described herein the VHH domain comprises a CDR3 sequence present in VHH10, e.g., the CDR3 sequence of DPFNQGY (SEQ ID NO: 68). In various embodiments of the aspects described herein the VHH domain comprises a CDR3 sequence present in VHH11, e.g., the CDR3 sequence of DLAEYSGTYSSPADSPAGYDY (SEQ ID NO: 69). In various embodiments of the aspects described herein the VHH domain comprises a CDR3 sequence present in VHH12, e.g., the CDR3 sequence of ARYYVSGTYFPANY (SEQ ID NO: 70).

In various embodiments of the aspects described herein the VHH domain comprises a CDR3 sequence present in VHH1, e.g., the CDR3 sequence of GSIDLNWYGGMDY (SEQ ID NO: 71) or SIDLNWYGGMD (SEQ ID NO: 272). In various embodiments of the aspects described herein the VHH domain comprises a CDR3 sequence present in VHH2, e.g., the CDR3 sequence of TTVLTDPRVLNEYAT (SEQ ID NO: 72) or TVLTDPRVLNEYA (SEQ ID NO: 273). In various embodiments of the aspects described herein the VHH domain comprises a CDR3 sequence present in VHH3, e.g., the CDR3 sequence of DVFGSSGYVETY (SEQ ID NO: 73) or VFGSSGYVET (SEQ ID NO: 274). In various embodiments of the aspects described herein the VHH domain comprises a CDR3 sequence present in VHH4, e.g., the CDR3 sequence of PLTAR (SEQ ID NO: 74) or LTA (SEQ ID NO: 275). In various embodiments of the aspects described herein the VHH domain comprises a CDR3 sequence present in VHH5, e.g., the CDR3 sequence of DPFNQGY (SEQ ID NO: 75) or PFNQG (SEQ ID NO: 276). In various embodiments of the aspects described herein the VHH domain comprises a CDR3 sequence present in VHH6, e.g., the CDR3 sequence of PLTSR (SEQ ID NO: 76) or LTS (SEQ ID NO: 277). In various embodiments of the aspects described herein the VHH domain comprises a CDR3 sequence present in VHH7, e.g., the CDR3 sequence of MVNPIITAWGTIGVREIPDYDY (SEQ ID NO: 77) or VNPIITAWGTIGVREIPDYD (SEQ ID NO: 278). In various embodiments of the aspects described herein the VHH domain comprises a CDR3 sequence present in VHH9, e.g., the CDR3 sequence of DQRGY (SEQ ID NO: 78) or RG (SEQ ID NO: 279). In various embodiments of the aspects described herein the VHH domain comprises a CDR3 sequence present in VHH10, e.g., the CDR3 sequence of DPFNQGY (SEQ ID NO: 79) or PFNQG (SEQ ID NO: 280). In various embodiments of the aspects described herein the VHH domain comprises a CDR3 sequence present in VHH11, e.g., the CDR3 sequence of DLAEYSGTYSSPADSPAGYDY (SEQ ID NO: 80) or LAEYSGTYSSPADSPAGYD (SEQ ID NO: 281). In various embodiments of the aspects described herein the VHH domain comprises a CDR3 sequence present in VHH12, e.g., the CDR3 sequence of ARYYVSGTYFPANY (SEQ ID NO: 81) or RYYVSGTYFPAN (SEQ ID NO: 282).

In various embodiments of the aspects described herein the VHH domain comprises a CDR3 sequence present in VHH1, e.g., the CDR3 sequence of CAAGSIDLNWYGGMDY (SEQ ID NO: 82) or AAGSIDLNWYGGMDY (SEQ ID NO: 283). In various embodiments of the aspects described herein the VHH domain comprises a CDR3 sequence present in VHH2, e.g., the CDR3 sequence of CAATTVLTDPRVLNEYAT (SEQ ID NO: 83) or AATTVLTDPRVLNEYAT (SEQ ID NO: 284). In various embodiments of the aspects described herein the VHH domain comprises a CDR3 sequence present in VHH3, e.g., the CDR3 sequence of KADVFGSSGYVETY (SEQ ID NO: 84). In various embodiments of the aspects described herein the VHH domain comprises a CDR3 sequence present in VHH4, e.g., the CDR3 sequence of NHPLTAR (SEQ ID NO: 85). In various embodiments of the aspects described herein the VHH domain comprises a CDR3 sequence present in VHH5, e.g., the CDR3 sequence of AADPFNQGY (SEQ ID NO: 86). In various embodiments of the aspects described herein the VHH domain comprises a CDR3 sequence present in VHH6, e.g., the CDR3 sequence of NHPLTSR (SEQ ID NO: 87). In various embodiments of the aspects described herein the VHH domain comprises a CDR3 sequence present in VHH7, e.g., the CDR3 sequence of ASMVNPIITAWGTIGVREIPDYDY (SEQ ID NO: 88). In various embodiments of the aspects described herein the VHH domain comprises a CDR3 sequence present in VHH9, e.g., the CDR3 sequence of NDQRGY (SEQ ID NO: 89). In various embodiments of the aspects described herein the VHH domain comprises a CDR3 sequence present in VHH10, e.g., the CDR3 sequence of AADPFNQGY (SEQ ID NO: 90). In various embodiments of the aspects described herein the VHH domain comprises a CDR3 sequence present in VHH11, e.g., the CDR3 sequence of AADLAEYSGTYSSPADSPAGYDY (SEQ ID NO: 91). In various embodiments of the aspects described herein the VHH domain comprises a CDR3 sequence present in VHH12, e.g., the CDR3 sequence of AAARYYVSGTYFPANY (SEQ ID NO: 92).

In various embodiments of the aspects described herein the VHH domain comprises a CDR3 sequence present in VHH1, e.g., the CDR3 sequence of GSIDLNWYGGMDY (SEQ ID NO: 214). In various embodiments of the aspects described herein the VHH domain comprises a CDR3 sequence present in VHH2, e.g., the CDR3 sequence of TTVLTDPRVLNEYAT (SEQ ID NO: 215). In various embodiments of the aspects described herein the VHH domain comprises a CDR3 sequence present in VHH3, e.g., the CDR3 sequence of DVFGSSGYVETY (SEQ ID NO: 216). In various embodiments of the aspects described herein the VHH domain comprises a CDR3 sequence present in VHH4, e.g., the CDR3 sequence of PLTAR (SEQ ID NO: 217). In various embodiments of the aspects described herein the VHH domain comprises a CDR3 sequence present in VHH5, e.g., the CDR3 sequence of DPFNQGY (SEQ ID NO: 218). In various embodiments of the aspects described herein the VHH domain comprises a CDR3 sequence present in VHH6, e.g., the CDR3 sequence of PLTSR (SEQ ID NO: 219). In various embodiments of the aspects described herein the VHH domain comprises a CDR3 sequence present in VHH7, e.g., the CDR3 sequence of MVNPIITAWGTIGVREIPDYDY (SEQ ID NO: 220). In various embodiments of the aspects described herein the VHH domain comprises a CDR3 sequence present in VHH9, e.g., the CDR3 sequence of QRGY (SEQ ID NO: 221). In various embodiments of the aspects described herein the VHH domain comprises a CDR3 sequence present in VHH10, e.g., the CDR3 sequence of DPFNQGY (SEQ ID NO: 222). In various embodiments of the aspects described herein the VHH domain comprises a CDR3 sequence present in VHH11, e.g., the CDR3 sequence of DLAEYSGTYSSPADSPAGYDY (SEQ ID NO: 223). In various embodiments of the aspects described herein the VHH domain comprises a CDR3 sequence present in VHH12, e.g., the CDR3 sequence of ARYYVSGTYFPANY (SEQ ID NO: 224).

In various embodiments of the aspects described herein the VHH domain comprises a CDR3 sequence present in VHH1, e.g., the CDR3 sequence of AAGSIDLNWYGGMD (SEQ ID NO: 225). In various embodiments of the aspects described herein the VHH domain comprises a CDR3 sequence present in VHH2, e.g., the CDR3 sequence of AATTVLTDPRVLNEYA (SEQ ID NO: 226). In various embodiments of the aspects described herein the VHH domain comprises a CDR3 sequence present in VHH3, e.g., the CDR3 sequence of KADVFGSSGYVET (SEQ ID NO: 227). In various embodiments of the aspects described herein the VHH domain comprises a CDR3 sequence present in VHH4, e.g., the CDR3 sequence of NHPLTA (SEQ ID NO: 228). In various embodiments of the aspects described herein the VHH domain comprises a CDR3 sequence present in VHH5, e.g., the CDR3 sequence of AADPFNQG (SEQ ID NO: 229). In various embodiments of the aspects described herein the VHH domain comprises a CDR3 sequence present in VHH6, e.g., the CDR3 sequence of NHPLTS (SEQ ID NO: 230). In various embodiments of the aspects described herein the VHH domain comprises a CDR3 sequence present in VHH7, e.g., the CDR3 sequence of ASMVNPIITAWGTIGVREIPDYD (SEQ ID NO: 231). In various embodiments of the aspects described herein the VHH domain comprises a CDR3 sequence present in VHH9, e.g., the CDR3 sequence of NDQRG (SEQ ID NO: 232). In various embodiments of the aspects described herein the VHH domain comprises a CDR3 sequence present in VHH10, e.g., the CDR3 sequence of AADPFNQG (SEQ ID NO: 233). In various embodiments of the aspects described herein the VHH domain comprises a CDR3 sequence present in VHH11, e.g., the CDR3 sequence of AADLAEYSGTYSSPADSPAGYD (SEQ ID NO: 234). In various embodiments of the aspects described herein the VHH domain comprises a CDR3 sequence present in VHH12, e.g., the CDR3 sequence of AAARYYVSGTYFPAN (SEQ ID NO: 235).

In various embodiments of the aspects described herein the VHH domain comprises a CDR3 sequence present in VHH1, e.g., the CDR3 sequence of GSIDLNWYGGMDY (SEQ ID NO: 236). In various embodiments of the aspects described herein the VHH domain comprises a CDR3 sequence present in VHH2, e.g., the CDR3 sequence of TTVLTDPRVLNEYAT (SEQ ID NO: 237). In various embodiments of the aspects described herein the VHH domain comprises a CDR3 sequence present in VHH3, e.g., the CDR3 sequence of DVFGSSGYVETY (SEQ ID NO: 238). In various embodiments of the aspects described herein the VHH domain comprises a CDR3 sequence present in VHH4, e.g., the CDR3 sequence of PLTAR (SEQ ID NO: 239). In various embodiments of the aspects described herein the VHH domain comprises a CDR3 sequence present in VHH5, e.g., the CDR3 sequence of DPFNQGY (SEQ ID NO: 240). In various embodiments of the aspects described herein the VHH domain comprises a CDR3 sequence present in VHH6, e.g., the CDR3 sequence of PLTSR (SEQ ID NO: 241). In various embodiments of the aspects described herein the VHH domain comprises a CDR3 sequence present in VHH7, e.g., the CDR3 sequence of MVNPIITAWGTIGVREIPDYDY (SEQ ID NO: 242). In various embodiments of the aspects described herein the VHH domain comprises a CDR3 sequence present in VHH9, e.g., the CDR3 sequence of QRGY (SEQ ID NO: 243). In various embodiments of the aspects described herein the VHH domain comprises a CDR3 sequence present in VHH10, e.g., the CDR3 sequence of DPFNQGY (SEQ ID NO: 244). In various embodiments of the aspects described herein the VHH domain comprises a CDR3 sequence present in VHH11, e.g., the CDR3 sequence of DLAEYSGTYSSPADSPAGYDY (SEQ ID NO: 245). In various embodiments of the aspects described herein the VHH domain comprises a CDR3 sequence present in VHH12, e.g., the CDR3 sequence of ARYYVSGTYFPANY (SEQ ID NO: 246).

In various embodiments of the aspects described herein, the VHH domain comprises a CDR1 sequence, a CDR2 sequence, and a CDR3 sequence of the VHH domain described herein. Accordingly, in some embodiments, the VHH domain comprises: i) the CDR1 sequence of SYRMG (SEQ ID NO: 1), the CDR2 sequence of AIDWNGRGTYYRYYADSVKG (SEQ ID NO: 30), and the CDR3 sequence of GSIDLNWYGGMDY (SEQ ID NO: 60); ii) the CDR1 sequence of GLTFSSY (SEQ ID NO: 10), the CDR2 sequence of DWNGRGTYY (SEQ ID NO: 40) or WNGRGTY (SEQ ID NO: 260), and the CDR3 sequence of GSIDLNWYGGMDY (SEQ ID NO: 71) or SIDLNWYGGMD (SEQ ID NO: 272); iii) the CDR1 sequence of GLTFSSYR (SEQ ID NO: 20), the CDR2 sequence of IDWNGRGTYY (SEQ ID NO: 50) or IDWNGRGTYYR (SEQ ID NO: 270), and the CDR3 sequence of CAAGSIDLNWYGGMDY (SEQ ID NO: 82) or AAGSIDLNWYGGMDY (SEQ ID NO: 283); iv) the CDR1 sequence of GLTFSSYRMG (SEQ ID NO: 154), the CDR 2 sequence of AIDWNGRGTYYRYYADSVKG (SEQ ID NO: 184), and the CDR3 sequence of GSIDLNWYGGMDY (SEQ ID NO: 214); v) the CDR1 sequence of SSYRMG (SEQ ID NO: 164), the CDR2 sequence of FVAAIDWNGRGTYYRY (SEQ ID NO: 194), and the CDR3 sequence of AAGSIDLNWYGGMD (SEQ ID NO: 225); or vi) the CDR1 sequence of GLTFSSYRMG (SEQ ID NO: 174), the CDR2 sequence of AIDWNGRGTYYRY (SEQ ID NO: 204), and the CDR3 sequence of GSIDLNWYGGMDY (SEQ ID NO: 236). In some embodiments, the VHH domain comprises: i) the CDR1 sequence of SYRMG (SEQ ID NO: 1), the CDR2 sequence of AIDWNGRGTYYRYYADSVKG (SEQ ID NO: 30), and the CDR3 sequence of TTVLTDPRVLNEYAT (SEQ ID NO: 61); ii) the CDR1 sequence of GLTFSSY (SEQ ID NO: 10), the CDR2 sequence of DWNGRGTYY (SEQ ID NO: 40) or WNGRGTY (SEQ ID NO: 260), and the CDR3 sequence of TTVLTDPRVLNEYAT (SEQ ID NO: 72) or TVLTDPRVLNEYA (SEQ ID NO: 273); iii) the CDR1 sequence of GLTFSSYR (SEQ ID NO: 20), the CDR2 sequence of IDWNGRGTYY (SEQ ID NO: 50) or IDWNGRGTYYR (SEQ ID NO: 270), and the CDR3 sequence of CAATTVLTDPRVLNEYAT (SEQ ID NO: 83) or AATTVLTDPRVLNEYAT (SEQ ID NO: 284); iv) the CDR1 sequence of GLTFSSYRMG (SEQ ID NO: 154), the CDR2 sequence of AIDWNGRGTYYRYYADSVKG (SEQ ID NO: 184), and the CDR3 sequence of TTVLTDPRVLNEYAT (SEQ ID NO: 215); v) the CDR1 sequence of SSYRMG (SEQ ID NO: 164), the CDR2 sequence of FVAAIDWNGRGTYYRY (SEQ ID NO: 194), and the CDR3 sequence of AATTVLTDPRVLNEYA (SEQ ID NO: 226); or vi) the CDR1 sequence of GLTFSSYRMG (SEQ ID NO: 174), the CDR2 sequence of AIDWNGRGTYYRY (SEQ ID NO: 204), and the CDR3 sequence of TTVLTDPRVLNEYAT (SEQ ID NO: 237). In some embodiments, the VHH domain comprises: i) the CDR1 sequence of INVMG (SEQ ID NO: 2), the CDR2 sequence of RINGGGITHYAESVKG (SEQ ID NO: 31), and the CDR3 sequence of DVFGSSGYVETY (SEQ ID NO: 62); ii) the CDR1 sequence of GSIFSIN (SEQ ID NO: 11), the CDR2 sequence of NGGGI (SEQ ID NO: 41) or GGG (SEQ ID NO: 261), and the CDR3 sequence of DVFGSSGYVETY (SEQ ID NO: 73) or VFGSSGYVET (SEQ ID NO: 274); iii) the CDR1 sequence of GSIFSINV (SEQ ID NO: 21), the CDR2 sequence of INGGGIT (SEQ ID NO: 51), and the CDR3 sequence of KADVFGSSGYVETY (SEQ ID NO: 84); iv) the CDR1 sequence of GSIFSINVMG (SEQ ID NO: 155), the CDR2 sequence of RINGGGITHYAESVKG (SEQ ID NO: 185), and the CDR3 sequence of DVFGSSGYVETY (SEQ ID NO: 216); v) the CDR1 sequence of SINVMG (SEQ ID NO: 165), the CDR2 sequence of LVARINGGGITH (SEQ ID NO: 195), and the CDR3 sequence of KADVFGSSGYVET (SEQ ID NO: 227); or vi) the CDR1 sequence of GSIFSINVMG (SEQ ID NO: 175), the CDR2 sequence of RINGGGITH (SEQ ID NO: 205), and the CDR3 sequence of DVFGSSGYVETY (SEQ ID NO: 238). In some embodiments, the VHH domain comprises: i) the CDR1 sequence of SNAMG (SEQ ID NO: 3), the CDR2 sequence of FIDRIATTTIATSVKG (SEQ ID NO: 32), and the CDR3 sequence of PLTAR (SEQ ID NO: 63); ii) the CDR1 sequence of GTSVSSN (SEQ ID NO: 12), the CDR2 sequence of DRIAT (SEQ ID NO: 42) or RIA (SEQ ID NO: 262), and the CDR3 sequence of PLTAR (SEQ ID NO: 74) or LTA (SEQ ID NO: 275); iii) the CDR1 sequence of GTSVSSNA (SEQ ID NO: 22), the CDR2 sequence of IDRIATT (SEQ ID NO: 52), and the CDR3 sequence of NHPLTAR (SEQ ID NO: 85); iv) the CDR1 sequence of GTSVSSNAMG (SEQ ID NO: 156), the CDR2 sequence of FIDRIATTTIATSVKG (SEQ ID NO: 186), and the CDR3 sequence of PLTAR (SEQ ID NO: 217); v) the CDR1 sequence of SSNAMG (SEQ ID NO: 166), the CDR2 sequence of WVGFIDRIATTT (SEQ ID NO: 196), and the CDR3 sequence of NHPLTA (SEQ ID NO: 228); or vi) the CDR1 sequence of GTSVSSNAMG (SEQ ID NO: 176), the CDR2 sequence of FIDRIATTT (SEQ ID NO: 206), and the CDR3 sequence of PLTAR (SEQ ID NO: 239). In some embodiments, the VHH domain comprises: i) the CDR1 sequence of SYAMG (SEQ ID NO: 4), the CDR2 sequence of AITWNGGTTYYADSVKG (SEQ ID NO: 33), and the CDR3 sequence of DPFNQGY (SEQ ID NO: 64); ii) the CDR1 sequence of GRTFSSY (SEQ ID NO: 13), the CDR2 sequence of TWNGGT (SEQ ID NO: 43) or WNGG (SEQ ID NO: 263), and the CDR3 sequence of DPFNQGY (SEQ ID NO: 75) or PFNQG (SEQ ID NO: 276); iii) the CDR1 sequence of GRTFSSYA (SEQ ID NO: 23), the CDR2 sequence of ITWNGGTT (SEQ ID NO: 53), and the CDR3 sequence of AADPFNQGY (SEQ ID NO: 86); iv) the CDR1 sequence of GRTFSSYAMG (SEQ ID NO: 157), the CDR2 sequence of AITWNGGTTYYADSVKG (SEQ ID NO: 187), and the CDR3 sequence of DPFNQGY (SEQ ID NO: 218); v) the CDR1 sequence of SSYAMG (SEQ ID NO: 167), the CDR2 sequence of FVAAITWNGGTTY (SEQ ID NO: 197), and the CDR3 sequence of AADPFNQG (SEQ ID NO: 229); or vi) the CDR1 sequence of GRTFSSYAMG (SEQ ID NO: 177), the CDR2 sequence of AITWNGGTTY (SEQ ID NO: 207), and the CDR3 sequence of DPFNQGY (SEQ ID NO: 240). In some embodiments, the VHH domain comprises: i) the CDR1 sequence of SDAMG (SEQ ID NO: 5), the CDR2 sequence of FISGGGTTTYADSVKG (SEQ ID NO: 34), and the CDR3 sequence of PLTSR (SEQ ID NO: 65); ii) the CDR1 sequence of GSSVSSD (SEQ ID NO: 14), the CDR2 sequence of SGGGT (SEQ ID NO: 44) or GGG (SEQ ID NO: 264), and the CDR3 sequence of PLTSR (SEQ ID NO: 76) or LTS (SEQ ID NO: 277); iii) the CDR1 sequence of GSSVSSDA (SEQ ID NO: 24), the CDR2 sequence of ISGGGTT (SEQ ID NO: 54), and the CDR3 sequence of NHPLTSR (SEQ ID NO: 87); iv) the CDR1 sequence of GSSVSSDAMG (SEQ ID NO: 158), the CDR2 sequence of FISGGGTTTYADSVKG (SEQ ID NO: 188), and the CDR3 sequence of PLTSR (SEQ ID NO: 219); v) the CDR1 sequence of SSDAMG (SEQ ID NO: 168), the CDR2 sequence of WVAFISGGGTTT (SEQ ID NO: 198), and the CDR3 sequence of NHPLTS (SEQ ID NO: 230); or vi) the CDR1 sequence of GSSVSSDAMG (SEQ ID NO: 178), the CDR2 sequence of FISGGGTTT (SEQ ID NO: 208), and the CDR3 sequence of PLTSR (SEQ ID NO: 241). In some embodiments, the VHH domain comprises: i) the CDR1 sequence of INVMG (SEQ ID NO: 6), the CDR2 sequence of RITGGGSTHYAESVKG (SEQ ID NO: 35), and the CDR3 sequence of MVNPIITAWGTIGVREIPDYDY (SEQ ID NO: 66); ii) the CDR1 sequence of RSIGSIN (SEQ ID NO: 15), the CDR2 sequence of TGGGS (SEQ ID NO: 45) or GGG (SEQ ID NO: 265), and the CDR3 sequence of MVNPIITAWGTIGVREIPDYDY (SEQ ID NO: 77) or VNPIITAWGTIGVREIPDYD (SEQ ID NO: 278); iii) the CDR1 sequence of RSIGSINV (SEQ ID NO: 25), the CDR2 sequence of ITGGGST (SEQ ID NO: 55), and the CDR3 sequence of ASMVNPIITAWGTIGVREIPDYDY (SEQ ID NO: 88); iv) the CDR1 sequence of RSIGSINVMG (SEQ ID NO: 159), the CDR2 sequence of RITGGGSTHYAESVKG (SEQ ID NO: 189), and the CDR3 sequence of MVNPIITAWGTIGVREIPDYDY (SEQ ID NO: 220); v) the CDR1 sequence of SINVMG (SEQ ID NO: 169), the CDR2 sequence of LVARITGGGSTH (SEQ ID NO: 199), and the CDR3 sequence of ASMVNPIITAWGTIGVREIPDYD (SEQ ID NO: 231); or vi) the CDR1 sequence of RSIGSINVMG (SEQ ID NO: 179), the CDR2 sequence of RITGGGSTH (SEQ ID NO: 209), and the CDR3 sequence of MVNPIITAWGTIGVREIPDYDY (SEQ ID NO: 242). In some embodiments, the VHH domain comprises: i) the CDR1 sequence of TYRMG (SEQ ID NO: 7), the CDR2 sequence of AISWSGGSTTYADPVKG (SEQ ID NO: 36), and the CDR3 sequence of DQRGY (SEQ ID NO: 67) or QRGY (SEQ ID NO: 271); ii) the CDR1 sequence of GRTFSTY (SEQ ID NO: 16), the CDR2 sequence of SWSGGS (SEQ ID NO: 46) or WSGG (SEQ ID NO: 266), and the CDR3 sequence of DQRGY (SEQ ID NO: 78) or RG (SEQ ID NO: 279); iii) the CDR1 sequence of GRTFSTYR (SEQ ID NO: 26), the CDR2 sequence of ISWSGGST (SEQ ID NO: 56), and the CDR3 sequence of NDQRGY (SEQ ID NO: 89); iv) the CDR1 sequence of GRTFSTYRMG (SEQ ID NO: 160), the CDR2 sequence of AISWSGGSTTYADPVKG (SEQ ID NO: 190), and the CDR3 sequence of QRGY (SEQ ID NO: 221); v) the CDR1 sequence of STYRMG (SEQ ID NO: 170), the CDR2 sequence of FVAAISWSGGSTT (SEQ ID NO: 200), and the CDR3 sequence of NDQRG (SEQ ID NO: 232); or vi) the CDR1 sequence of GRTFSTYRMG (SEQ ID NO: 180), the CDR2 sequence of AISWSGGSTT (SEQ ID NO: 210), and the CDR3 sequence of QRGY (SEQ ID NO: 243). In some embodiments, the VHH domain comprises: i) the CDR1 sequence of RYAMG (SEQ ID NO: 8), the CDR2 sequence of AISWSGSSAGYGDSVKG (SEQ ID NO: 37), and the CDR3 sequence of DPFNQGY (SEQ ID NO: 68); ii) the CDR1 sequence of GFTFTRY (SEQ ID NO: 17), the CDR2 sequence of SWSGSS (SEQ ID NO: 47) or WSGS (SEQ ID NO: 267), and the CDR3 sequence of DPFNQGY (SEQ ID NO: 79) or PFNQG (SEQ ID NO: 280); iii) the CDR1 sequence of GFTFTRYA (SEQ ID NO: 27), the CDR2 sequence of ISWSGSSA (SEQ ID NO: 57), and the CDR3 sequence of AADPFNQGY (SEQ ID NO: 90); iv) the CDR1 sequence of GFTFTRYAMG (SEQ ID NO: 161), the CDR2 sequence of AISWSGSSAGYGDSVKG (SEQ ID NO: 191), and the CDR3 sequence of DPFNQGY (SEQ ID NO: 222); v) the CDR1 sequence of TRYAMG (SEQ ID NO: 171), the CDR2 sequence of FVAAISWSGSSAG (SEQ ID NO: 201), and the CDR3 sequence of AADPFNQG (SEQ ID NO: 233); or vi) the CDR1 sequence of GFTFTRYAMG (SEQ ID NO: 181), the CDR2 sequence of AISWSGSSAG (SEQ ID NO: 211), and the CDR3 sequence of DPFNQGY (SEQ ID NO: 244). In some embodiments, the VHH domain comprises: i) the CDR1 sequence of FTTYRMG (SEQ ID NO: 258) or TYRMG (SEQ ID NO: 259), the CDR2 sequence of AIRWSGGRTLYADSVKG (SEQ ID NO: 38), and the CDR3 sequence of DLAEYSGTYSSPADSPAGYDY (SEQ ID NO: 69); ii) the CDR1 sequence of GRTFTTY (SEQ ID NO: 18), the CDR2 sequence of RWSGGR (SEQ ID NO: 48) or WSGG (SEQ ID NO: 268), and the CDR3 sequence of DLAEYSGTYSSPADSPAGYDY (SEQ ID NO: 80) or LAEYSGTYSSPADSPAGYD (SEQ ID NO: 281); iii) the CDR1 sequence of GRTFTTYR (SEQ ID NO: 28), the CDR2 sequence of IRWSGGRT (SEQ ID NO: 58), and the CDR3 sequence of AADLAEYSGTYSSPADSPAGYDY (SEQ ID NO: 91); iv) the CDR1 sequence of GRTFTTYRMG (SEQ ID NO: 162), the CDR2 sequence of AIRWSGGRTLYADSVKG (SEQ ID NO: 192), and the CDR3 sequence of DLAEYSGTYSSPADSPAGYDY (SEQ ID NO: 223); v) the CDR1 sequence of TTYRMG (SEQ ID NO: 172), the CDR2 sequence of FVAAIRWSGGRTL (SEQ ID NO: 202), and the CDR3 sequence of AADLAEYSGTYSSPADSPAGYD (SEQ ID NO: 234); or vi) the CDR1 sequence of GRTFTTYRMG (SEQ ID NO: 182), the CDR2 sequence of AIRWSGGRTL (SEQ ID NO: 212), and the CDR3 sequence of DLAEYSGTYSSPADSPAGYDY (SEQ ID NO: 245). In some embodiments, the VHH domain comprises: i) the CDR1 sequence of FNTYAMG (SEQ ID NO: 9), the CDR2 sequence of SITWNGGSTSYADSVKG (SEQ ID NO: 39), and the CDR3 sequence of ARYYVSGTYFPANY (SEQ ID NO: 70); ii) the CDR1 sequence of GRTLSFNTY (SEQ ID NO: 19), the CDR2 sequence of TWNGGS (SEQ ID NO: 49) or WNGG (SEQ ID NO: 269), and the CDR3 sequence of ARYYVSGTYFPANY (SEQ ID NO: 81) or RYYVSGTYFPAN (SEQ ID NO: 282); iii) the CDR1 sequence of GRTLSFNTYA (SEQ ID NO: 29), the CDR2 sequence of ITWNGGST (SEQ ID NO: 59), and the CDR3 sequence of AAARYYVSGTYFPANY (SEQ ID NO: 92); iv) the CDR1 sequence of GRTLSFNTYAMG (SEQ ID NO: 163), the CDR2 sequence of SITWNGGSTSYADSVKG (SEQ ID NO: 193), and the CDR3 sequence of ARYYVSGTYFPANY (SEQ ID NO: 224); v) the CDR1 sequence of SFNTYAMG (SEQ ID NO: 173), the CDR2 sequence of FVASITWNGGSTS (SEQ ID NO: 203), and the CDR3 sequence of AAARYYVSGTYFPAN (SEQ ID NO: 235); or vi) the CDR1 sequence of GRTLSFNTYAMG (SEQ ID NO: 183), the CDR2 sequence of SITWNGGSTS (SEQ ID NO: 213), and the CDR3 sequence of ARYYVSGTYFPANY (SEQ ID NO: 246).

In one aspect, provided herein is a VHH domain that binds to domain 1 of pIgR, wherein the VHH domain comprises the CDR1, CDR2 and/or CDR3 sequence of VHH2 or VHH3 described herein. Accordingly, in some embodiments, the VHH domain that bind to domain 1 of pIgR comprises the CDR1, CDR2 and CDR3 sequence of:
a) VHH2:
   i) the CDR1 sequence of SYRMG (SEQ ID NO: 1), the CDR2 sequence of AIDWNGRGTYYRYYADSVKG (SEQ ID NO: 30), and the CDR3 sequence of TTVLTDPRVLNEYAT (SEQ ID NO: 61);
   ii) the CDR1 sequence of GLTFSSY (SEQ ID NO: 10), the CDR2 sequence of DWNGRGTYY (SEQ ID NO: 40) or WNGRGTY (SEQ ID NO: 260), and the CDR3 sequence of TTVLTDPRVLNEYAT (SEQ ID NO: 72) or TVLTDPRVLNEYA (SEQ ID NO: 273);
   iii) the CDR1 sequence of GLTFSSYR (SEQ ID NO: 20), the CDR2 sequence of IDWNGRGTYY (SEQ ID NO: 50) or IDWNGRGTYYR (SEQ ID NO: 270), and the CDR3 sequence of CAATTVLTDPRVLNEYAT (SEQ ID NO: 83) or AATTVLTDPRVLNEYAT (SEQ ID NO: 284);
   iv) the CDR1 sequence of GLTFSSYRMG (SEQ ID NO: 154), the CDR2 sequence of AIDWNGRGTYYRYYADSVKG (SEQ ID NO: 184), and the CDR3 sequence of TTVLTDPRVLNEYAT (SEQ ID NO: 215);
   v) the CDR1 sequence of SSYRMG (SEQ ID NO: 164), the CDR2 sequence of FVAAIDWNGRGTYYRY (SEQ ID NO: 194), and the CDR3 sequence of AATTVLTDPRVLNEYA (SEQ ID NO: 226); or
   vi) the CDR1 sequence of GLTFSSYRMG (SEQ ID NO: 174), the CDR2 sequence of AIDWNGRGTYYRY (SEQ ID NO: 204), and the CDR3 sequence of TTVLTDPRVLNEYAT (SEQ ID NO: 237); or
b) VHH3:
   i) the CDR1 sequence of INVMG (SEQ ID NO: 2), the CDR2 sequence of RINGGGITHYAESVKG (SEQ ID NO: 31), and the CDR3 sequence of DVFGSSGYVETY (SEQ ID NO: 62);
   ii) the CDR1 sequence of GSIFSIN (SEQ ID NO: 11), the CDR2 sequence of NGGGI (SEQ ID NO: 41) or GGG (SEQ ID NO: 261), and the CDR3 sequence of DVFGSSGYVETY (SEQ ID NO: 73) or VFGSSGYVET (SEQ ID NO: 274);
   iii) the CDR1 sequence of GSIFSINV (SEQ ID NO: 21), the CDR2 sequence of INGGGIT (SEQ ID NO: 51), and the CDR3 sequence of KADVFGSSGYVETY (SEQ ID NO: 84);
   iv) the CDR1 sequence of GSIFSINVMG (SEQ ID NO: 155), the CDR2 sequence of RINGGGITHYAESVKG (SEQ ID NO: 185), and the CDR3 sequence of DVFGSSGYVETY (SEQ ID NO: 216);
   v) the CDR1 sequence of SINVMG (SEQ ID NO: 165), the CDR2 sequence of LVARINGGGITH (SEQ ID NO: 195), and the CDR3 sequence of KADVFGSSGYVET (SEQ ID NO: 227); or
   vi) the CDR1 sequence of GSIFSINVMG (SEQ ID NO: 175), the CDR2 sequence of RINGGGITH (SEQ ID NO: 205), and the CDR3 sequence of DVFGSSGYVETY (SEQ ID NO: 238).

In one aspect, provided herein is a VHH domain that binds to domain 2 of pIgR, wherein the VHH domain comprises the CDR1, CDR2 and/or CDR3 sequence of VHH4 or VHH6 described herein. Accordingly, in some embodiments, the VHH domain that bind to domain 2 of pIgR comprises the CDR1, CDR2 and CDR3 sequence of:
a) VHH4:
   i) the CDR1 sequence of SNAMG (SEQ ID NO: 3), the CDR2 sequence of FIDRIATTTIATSVKG (SEQ ID NO: 32), and the CDR3 sequence of PLTAR (SEQ ID NO: 63);
   ii) the CDR1 sequence of GTSVSSN (SEQ ID NO: 12), the CDR2 sequence of DRIAT (SEQ ID NO: 42) or RIA (SEQ ID NO: 262), and the CDR3 sequence of PLTAR (SEQ ID NO: 74) or LTA (SEQ ID NO: 275);
   iii) the CDR1 sequence of GTSVSSNA (SEQ ID NO: 22), the CDR2 sequence of IDRIATT (SEQ ID NO: 52), and the CDR3 sequence of NHPLTAR (SEQ ID NO: 85); iv) the CDR1 sequence of GTSVSSNAMG (SEQ ID NO: 156), the CDR2 sequence of FIDRIATTTIATSVKG (SEQ ID NO: 186), and the CDR3 sequence of PLTAR (SEQ ID NO: 217);
   v) the CDR1 sequence of SSNAMG (SEQ ID NO: 166), the CDR2 sequence of WVGFIDRIATTT (SEQ ID NO: 196), and the CDR3 sequence of NHPLTA (SEQ ID NO: 228); or
   vi) the CDR1 sequence of GTSVSSNAMG (SEQ ID NO: 176), the CDR2 sequence of FIDRIATTT (SEQ ID NO: 206), and the CDR3 sequence of PLTAR (SEQ ID NO: 239); or
b) VHH6:
   i) the CDR1 sequence of SDAMG (SEQ ID NO: 5), the CDR2 sequence of FISGGGTTTYADSVKG (SEQ ID NO: 34), and the CDR3 sequence of PLTSR (SEQ ID NO: 65);
   ii) the CDR1 sequence of GSSVSSD (SEQ ID NO: 14), the CDR2 sequence of SGGGT (SEQ ID NO: 44) or GGG (SEQ ID NO: 264), and the CDR3 sequence of PLTSR (SEQ ID NO: 76) or LTS (SEQ ID NO: 277);
   iii) the CDR1 sequence of GSSVSSDA (SEQ ID NO: 24), the CDR2 sequence of ISGGGTT (SEQ ID NO: 54), and the CDR3 sequence of NHPLTSR (SEQ ID NO: 87); iv) the CDR1 sequence of GSSVSSDAMG (SEQ ID NO: 158), the CDR2 sequence of FISGGGTTTYADSVKG (SEQ ID NO: 188), and the CDR3 sequence of PLTSR (SEQ ID NO: 219);
   v) the CDR1 sequence of SSDAMG (SEQ ID NO: 168), the CDR2 sequence of WVAFISGGGTTT (SEQ ID NO: 198), and the CDR3 sequence of NHPLTS (SEQ ID NO: 230); or
   vi) the CDR1 sequence of GSSVSSDAMG (SEQ ID NO: 178), the CDR2 sequence of FISGGGTTT (SEQ ID NO: 208), and the CDR3 sequence of PLTSR (SEQ ID NO: 241).

In one aspect, provided herein is a VHH domain that binds to domain 4-5 of pIgR, wherein the VHH domain comprises the CDR1, CDR2 and/or CDR3 sequence of VHH5, VHH7, VHH9, VHH10 or VHH11 described herein. Accordingly, in some embodiments, the VHH domain that bind to domain 4-5 of pIgR comprises the CDR1, CDR2 and CDR3 sequence of:
a) VHH5:
   i) the CDR1 sequence of SYAMG (SEQ ID NO: 4), the CDR2 sequence of AITWNGGTTYYADSVKG (SEQ ID NO: 33), and the CDR3 sequence of DPFNQGY (SEQ ID NO: 64);
   ii) the CDR1 sequence of GRTFSSY (SEQ ID NO: 13), the CDR2 sequence of TWNGGT (SEQ ID NO: 43) or WNGG (SEQ ID NO: 263), and the CDR3 sequence of DPFNQGY (SEQ ID NO: 75) or PFNQG (SEQ ID NO: 276);
   iii) the CDR1 sequence of GRTFSSYA (SEQ ID NO: 23), the CDR2 sequence of ITWNGGTT (SEQ ID NO: 53), and the CDR3 sequence of AADPFNQGY (SEQ ID NO: 86);
   iv) the CDR1 sequence of GRTFSSYAMG (SEQ ID NO: 157), the CDR2 sequence of AITWNGGTTYYADSVKG (SEQ ID NO: 187), and the CDR3 sequence of DPFNQGY (SEQ ID NO: 218);
   v) the CDR1 sequence of SSYAMG (SEQ ID NO: 167), the CDR2 sequence of FVAAITWNGGTTY (SEQ ID NO: 197), and the CDR3 sequence of AADPFNQG (SEQ ID NO: 229); or
   vi) the CDR1 sequence of GRTFSSYAMG (SEQ ID NO: 177), the CDR2 sequence of AITWNGGTTY (SEQ ID NO: 207), and the CDR3 sequence of DPFNQGY (SEQ ID NO: 240);
b) VHH7:
   i) the CDR1 sequence of INVMG (SEQ ID NO: 6), the CDR2 sequence of RITGGGSTHYAESVKG (SEQ ID NO: 35), and the CDR3 sequence of MVNPIITAWGTIGVREIPDYDY (SEQ ID NO: 66);
   ii) the CDR1 sequence of RSIGSIN (SEQ ID NO: 15), the CDR2 sequence of TGGGS (SEQ ID NO: 45) or GGG (SEQ ID NO: 265), and the CDR3 sequence of MVNPIITAWGTIGVREIPDYDY (SEQ ID NO: 77) or VNPIITAWGTIGVREIPDYD (SEQ ID NO: 278);
   iii) the CDR1 sequence of RSIGSINV (SEQ ID NO: 25), the CDR2 sequence of ITGGGST (SEQ ID NO: 55), and the CDR3 sequence of ASMVNPIITAWGTIGVREIPDYDY (SEQ ID NO: 88);
   iv) the CDR1 sequence of RSIGSINVMG (SEQ ID NO: 159), the CDR2 sequence of RITGGGSTHYAESVKG (SEQ ID NO: 189), and the CDR3 sequence of MVNPIITAWGTIGVREIPDYDY (SEQ ID NO: 220);
   v) the CDR1 sequence of SINVMG (SEQ ID NO: 169), the CDR2 sequence of LVARITGGGSTH (SEQ ID NO: 199), and the CDR3 sequence of ASMVNPIITAWGTIGVREIPDYD (SEQ ID NO: 231); or
   vi) the CDR1 sequence of RSIGSINVMG (SEQ ID NO: 179), the CDR2 sequence of RITGGGSTH (SEQ ID NO: 209), and the CDR3 sequence of MVNPIITAWGTIGVREIPDYDY (SEQ ID NO: 242);
c) VHH9:
   i) the CDR1 sequence of TYRMG (SEQ ID NO: 7), the CDR2 sequence of AISWSGGSTTYADPVKG (SEQ ID NO: 36), and the CDR3 sequence of DQRGY (SEQ ID NO: 67) or QRGY (SEQ ID NO: 271);
   ii) the CDR1 sequence of GRTFSTY (SEQ ID NO: 16), the CDR2 sequence of SWSGGS (SEQ ID NO: 46) or WSGG (SEQ ID NO: 266), and the CDR3 sequence of DQRGY (SEQ ID NO: 78) or RG (SEQ ID NO: 279);
   iii) the CDR1 sequence of GRTFSTYR (SEQ ID NO: 26), the CDR2 sequence of ISWSGGST (SEQ ID NO: 56), and the CDR3 sequence of NDQRGY (SEQ ID NO: 89); iv) the CDR1 sequence of GRTFSTYRMG (SEQ ID NO: 160), the CDR2 sequence of AISWSGGSTTYADPVKG (SEQ ID NO: 190), and the CDR3 sequence of QRGY (SEQ ID NO: 221);
   v) the CDR1 sequence of STYRMG (SEQ ID NO: 170), the CDR2 sequence of FVAAISWSGGSTT (SEQ ID NO: 200), and the CDR3 sequence of NDQRG (SEQ ID NO: 232); or
   vi) the CDR1 sequence of GRTFSTYRMG (SEQ ID NO: 180), the CDR2 sequence of AISWSGGSTT (SEQ ID NO: 210), and the CDR3 sequence of QRGY (SEQ ID NO: 243);
d) VHH10:
   i) the CDR1 sequence of RYAMG (SEQ ID NO: 8), the CDR2 sequence of AISWSGSSAGYGDSVKG (SEQ ID NO: 37), and the CDR3 sequence of DPFNQGY (SEQ ID NO: 68);
   ii) the CDR1 sequence of GFTFTRY (SEQ ID NO: 17), the CDR2 sequence of SWSGSS (SEQ ID NO: 47) or WSGS (SEQ ID NO: 267), and the CDR3 sequence of DPFNQGY (SEQ ID NO: 79) or PFNQG (SEQ ID NO: 280);
   iii) the CDR1 sequence of GFTFTRYA (SEQ ID NO: 27), the CDR2 sequence of ISWSGSSA (SEQ ID NO: 57), and the CDR3 sequence of AADPFNQGY (SEQ ID NO: 90); iv) the CDR1 sequence of GFTFTRYAMG (SEQ ID NO: 161), the CDR2 sequence of AISWSGSSAGYGDSVKG (SEQ ID NO: 191), and the CDR3 sequence of DPFNQGY (SEQ ID NO: 222);
   v) the CDR1 sequence of TRYAMG (SEQ ID NO: 171), the CDR2 sequence of FVAAISWSGSSAG (SEQ ID NO: 201), and the CDR3 sequence of AADPFNQG (SEQ ID NO: 233); or
   vi) the CDR1 sequence of GFTFTRYAMG (SEQ ID NO: 181), the CDR2 sequence of AISWSGSSAG (SEQ ID NO: 211), and the CDR3 sequence of DPFNQGY (SEQ ID NO: 244); or
e) VHH11:
   i) the CDR1 sequence of FTTYRMG (SEQ ID NO: 258) or TYRMG (SEQ ID NO: 259), the CDR2 sequence of AIRWSGGRTLYADSVKG (SEQ ID NO: 38), and the CDR3 sequence of DLAEYSGTYSSPADSPAGYDY (SEQ ID NO: 69);
   ii) the CDR1 sequence of GRTFTTY (SEQ ID NO: 18), the CDR2 sequence of RWSGGR (SEQ ID NO: 48) or WSGG (SEQ ID NO: 268), and the CDR3 sequence of DLAEYSGTYSSPADSPAGYDY (SEQ ID NO: 80) or LAEYSGTYSSPADSPAGYD (SEQ ID NO: 281);
   iii) the CDR1 sequence of GRTFTTYR (SEQ ID NO: 28), the CDR2 sequence of IRWSGGRT (SEQ ID NO: 58), and the CDR3 sequence of AADLAEYSGTYSSPADSPAGYDY (SEQ ID NO: 91);
   iv) the CDR1 sequence of GRTFTTYRMG (SEQ ID NO: 162), the CDR2 sequence of AIRWSGGRTLYADSVKG (SEQ ID NO: 192), and the CDR3 sequence of DLAEYSGTYSSPADSPAGYDY (SEQ ID NO: 223);
   v) the CDR1 sequence of TTYRMG (SEQ ID NO: 172), the CDR2 sequence of FVAAIRWSGGRTL (SEQ ID NO: 202), and the CDR3 sequence of AADLAEYSGTYSSPADSPAGYD (SEQ ID NO: 234); or
   vi) the CDR1 sequence of GRTFTTYRMG (SEQ ID NO: 182), the CDR2 sequence of AIRWSGGRTL (SEQ ID NO: 212), and the CDR3 sequence of DLAEYSGTYSSPADSPAGYDY (SEQ ID NO: 245).

In one aspect, provided herein is a VHH domain that binds to domain 5 of pIgR, wherein the VHH domain comprises the CDR1, CDR2 and/or CDR3 sequence of VHH12 described herein. Accordingly, in some embodiments, the VHH domain that bind to domain 5 of pIgR comprises the CDR1, CDR2 and CDR3 sequence of:
a) VHH12:
   i) the CDR1 sequence of FNTYAMG (SEQ ID NO: 9), the CDR2 sequence of SITWNGGSTSYADSVKG (SEQ ID NO: 39), and the CDR3 sequence of ARYYVSGTYFPANY (SEQ ID NO: 70);
   ii) the CDR1 sequence of GRTLSFNTY (SEQ ID NO: 19), the CDR2 sequence of TWNGGS (SEQ ID NO: 49) or WNGG (SEQ ID NO: 269), and the CDR3 sequence of ARYYVSGTYFPANY (SEQ ID NO: 81) or RYYVSGTYFPAN (SEQ ID NO: 282);
   iii) the CDR1 sequence of GRTLSFNTYA (SEQ ID NO: 29), the CDR2 sequence of ITWNGGST (SEQ ID NO: 59), and the CDR3 sequence of AAARYYVSGTYFPANY (SEQ ID NO: 92);
   iv) the CDR1 sequence of GRTLSFNTYAMG (SEQ ID NO: 163), the CDR2 sequence of SITWNGGSTSYADSVKG (SEQ ID NO: 193), and the CDR3 sequence of ARYYVSGTYFPANY (SEQ ID NO: 224);
   v) the CDR1 sequence of SFNTYAMG (SEQ ID NO: 173), the CDR2 sequence of FVASITWNGGSTS (SEQ ID NO: 203), and the CDR3 sequence of AAARYYVSGTYFPAN (SEQ ID NO: 235); or
   vi) the CDR1 sequence of GRTLSFNTYAMG (SEQ ID NO: 183), the CDR2 sequence of SITWNGGSTS (SEQ ID NO: 213), and the CDR3 sequence of ARYYVSGTYFPANY (SEQ ID NO: 246).

In various embodiments of the aspects described herein, the VHH domain comprises a framework derived from a VHH domain comprising the sequence of QVQLVESGGGLVQAGGSLKLACAAPGLTFSSYRMGWFRQAPGQEREFVAAIDWNGRG TYYRYYADSVKGRSTISRDNAKNTMYLQMNSLKPEDTAVYYCAAGSIDLNWYGGMD YWGQGTQVTVSS (SEQ ID NO: 93). In various embodiments of the aspects described herein, the VHH domain comprises a framework derived from a VHH domain comprising the sequence of EVQVVESGGGLVQAGGSLKLACAAPGLTFSSYRMGWFRQAPGQEREFVAAIDWNGRG TYYRYYADSVKGRSTISRDNAKNTVYLQMNSLKPEDTAVYYCAATTVLTDPRVLNEYA TWGQGTQVTVSS (SEQ ID NO: 94). In various embodiments of the aspects described herein, the VHH domain comprises a framework derived from a VHH domain comprising the sequence of QLQLVESGGGLVQPGGSLRLSCAASGSIFSINVMGWYRQAPGKQRELVARINGGGITHY AESVKGRFTISRDNAKNTVYLQMNSLKPEDTAAYYCKADVFGSSGYVETYWGQGTQV TVSS (SEQ ID NO: 95). In various embodiments of the aspects described herein, the VHH domain comprises a framework derived from a VHH domain comprising the sequence of EVQVVESGGGLVQAGGSLRLSCAVSGTSVSSNAMGWYRQAPGKQREWVGFIDRIATTT IATSVKGRFAITRDNAKNTVYLQMSGLKPEDTAVYYCNHPLTARWGQGTQVTVSS (SEQ ID NO: 96). In various embodiments of the aspects described herein, the VHH domain comprises a framework derived from a VHH domain comprising the sequence of QVQLVESGGGLVQAGGSLRLSCAASGRTFSSYAMGWFRQAPGKEREFVAAITWNGGTT YYADSVKGRFTISRDNAKNTVYLQMNSLKPEDTAVYYCAADPFNQGYWGQGTQVTVS S (SEQ ID NO: 97). In various embodiments of the aspects described herein, the VHH domain comprises a framework derived from a VHH domain comprising the sequence of EVQLVESGGGLVQAGGSLRLSCAVSGSSVSSDAMGWYRQAPGNQRAWVAFISGGGTT TYADSVKGRFTISRDNTKNTVYLHMNSLKPEDTAVYYCNHPLTSRWGQGTQVTVSS (SEQ ID NO: 98). In various embodiments of the aspects described herein, the VHH domain comprises a framework derived from a VHH domain comprising the sequence of EVQVVESGGGLVQAGGSLRLACVASRSIGSINVMGWYRQAPGKQRDLVARITGGGSTH YAESVKGRFTISRDNAKNTVYLQMNSLEPEDTAVYYCASMVNPIITAWGTIGVREIPDY DYWGQGTQVTVSS (SEQ ID NO: 99). In various embodiments of the aspects described herein, the VHH domain comprises a framework derived from a VHH domain comprising the sequence of QVQLVESGGGLVQAGGSLRLSCAVSGRTFSTYRMGWFRQAPGKERSFVAAISWSGGST TYADPVKGRFTISRDNAKNTVYLRMNSLKPEDTAVYYCNDQRGYWGQGTLVTVSS (SEQ ID NO: 100). In various embodiments of the aspects described herein, the VHH domain comprises a framework derived from a VHH domain comprising the sequence of EVQVVESGGGLVQAGGSLRLSCAASGFTFTRYAMGWFRQAPGKERSFVAAISWSGSSA GYGDSVKGRFTISRDNAKNTLYLQMNSLKPEDTAVYYCAADPFNQGYWGQGTQVTVS S (SEQ ID NO: 101). In various embodiments of the aspects described herein, the VHH domain comprises a framework derived from a VHH domain comprising the sequence of EVQVVESGGGLVQAGGSLRLSCAASGRTFTTYRMGWFRQAPGKEREFVAAIRWSGGRT LYADSVKGRFTISRDNAKNTAYLQMNNLRPEDTAVYYCAADLAEYSGTYSSPADSPAG YDYWGQGTQVTVSS (SEQ ID NO: 102). In various embodiments of the aspects described herein, the VHH domain comprises a framework derived from a VHH domain comprising the sequence of

(SEQ ID NO: 103)
QVQLVETGGGLVQAGDSLRLSCAASGRTLSFNTYAMGWFRQAPGKEREF
VASITWNGGSTSYADSVKGRFTITRDNAKNTATLRMNSLQPDDTAVYYC
AAARYYVSGTYFPANYWGQGTQVTVSS.

In various embodiments of the aspects described herein, the VHH domain comprises a framework comprising sequence having at least 75%, 80%, 85%, 90%, 95%, 96%, 97%, 98%, 99%, or 100% sequence identity with the sequence of QVQLVESGGGLVQAGGSLKLACAAPGLTFSSYRMGWFRQAPGQEREFVAAIDWNGRG TYYRYYADSVKGRSTISRDNAKNTMYLQMNSLKPEDTAVYYCAAGSIDLNWYGGMD YWGQGTQVTVSS (SEQ ID NO: 93). In various embodiments of the aspects described herein, the VHH domain comprises a framework comprising sequence having at least 75%, 80%, 85%, 90%, 95%, 96%, 97%, 98%, 99%, or 100% sequence identity with the sequence of EVQVVESGGGLVQAGGSLKLACAAPGLTFSSYRMGWFRQAPGQEREFVAAIDWNGRG TYYRYYADSVKGRSTISRDNAKNTVYLQMNSLKPEDTAVYYCAATTVLTDPRVLNEYA TWGQGTQVTVSS (SEQ ID NO: 94). In various embodiments of the aspects described herein, the VHH domain comprises a framework comprising sequence having at least 75%, 80%, 85%, 90%, 95%, 96%, 97%, 98%, 99%, or 100% sequence identity with the sequence of QLQLVESGGGLVQPGGSLRLSCAASGSIFSINVMGWYRQAPGKQRELVARINGGGITHY AESVKGRFTISRDNAKNTVYLQMNSLKPEDTAAYYCKADVFGSSGYVETYWGQGTQV TVSS (SEQ ID NO: 95). In various embodiments of the aspects described herein, the VHH domain comprises a framework comprising sequence having at least 75%, 80%, 85%, 90%, 95%, 96%, 97%, 98%, 99%, or 100% sequence identity with the sequence of EVQVVESGGGLVQAGGSLRLSCAVSGTSVSSNAMGWYRQAPGKQREWVGFIDRIATTT IATSVKGRFAITRDNAKNTVYLQMSGLKPEDTAVYYCNHPLTARWGQGTQVTVSS (SEQ ID NO: 96). In various embodiments of the aspects described herein, the VHH domain comprises a framework comprising sequence having at least 75%, 80%, 85%, 90%, 95%, 96%, 97%, 98%, 99%, or 100% sequence identity with the sequence of QVQLVESGGGLVQAGGSLRLSCAASGRTFSSYAMGWFRQAPGKEREFVAAITWNGGTT YYADSVKGRFTISRDNAKNTVYLQMNSLKPEDTAVYYCAADPFNQGYWGQGTQVTVS S (SEQ ID NO: 97). In various embodiments of the aspects described herein, the VHH domain comprises a framework comprising sequence having at least 75%, 80%, 85%, 90%, 95%, 96%, 97%, 98%, 99%, or 100% sequence identity with the sequence of EVQLVESGGGLVQAGGSLRLSCAVSGSSVSSDAMGWYRQAPGNQRAWVAFISGGGTT TYADSVKGRFTISRDNTKNTVYLHMNSLKPEDTAVYYCNHPLTSRWGQGTQVTVSS (SEQ ID NO: 98). In various embodiments of the aspects described herein, the VHH domain comprises a framework comprising sequence having at least 75%, 80%, 85%, 90%, 95%, 96%, 97%, 98%, 99%, or 100% sequence identity with the sequence of EVQVVESGGGLVQAGGSLRLACVASRSIGSINVMGWYRQAPGKQRDLVARITGGGSTH YAESVKGRFTISRDNAKNTVYLQMNSLEPEDTAVYYCASMVNPIITAWGTIGVREIPDY DYWGQGTQVTVSS (SEQ ID NO: 99). In various embodiments of the aspects described herein, the VHH domain comprises a framework comprising sequence having at least 75%, 80%, 85%, 90%, 95%, 96%, 97%, 98%, 99%, or 100% sequence identity with the sequence of QVQLVESGGGLVQAGGSLRLSCAVSGRTFSTYRMGWFRQAPGKERSFVAAISWSGGST TYADPVKGRFTISRDNAKNTVYLRMNSLKPEDTAVYYCNDQRGYWGQGTLVTVSS (SEQ ID NO: 100). In various embodiments of the aspects described herein, the VHH domain comprises a framework comprising sequence having at least 75%, 80%, 85%, 90%, 95%, 96%, 97%, 98%, 99%, or 100% sequence identity with the sequence of EVQVVESGGGLVQAGGSLRLSCAASGFTFTRYAMGWFRQAPGKERSFVAAISWSGSSA GYGDSVKGRFTISRDNAKNTLYLQMNSLKPEDTAVYYCAADPFNQGYWGQGTQVTVS S (SEQ ID NO: 101). In various embodiments of the aspects described herein, the VHH domain comprises a framework comprising sequence having at least 75%, 80%, 85%, 90%, 95%, 96%, 97%, 98%, 99%, or 100% sequence identity with the sequence of EVQVVESGGGLVQAGGSLRLSCAASGRTFTTYRMGWFRQAPGKEREFVAAIRWSGGRT LYADSVKGRFTISRDNAKNTAYLQMNNLRPEDTAVYYCAADLAEYSGTYSSPADSPAG YDYWGQGTQVTVSS (SEQ ID NO: 102). In various embodiments of the aspects described herein, the VHH domain comprises a framework comprising sequence having at least 75%, 80%, 85%, 90%, 95%, 96%, 97%, 98%, 99%, or 100% sequence identity with the sequence of

(SEQ ID NO: 103)
QVQLVETGGGLVQAGDSLRLSCAASGRTLSFNTYAMGWFRQAPGKEREF
VASITWNGGSTSYADSVKGRFTITRDNAKNTATLRMNSLQPDDTAVYYC
AAARYYVSGTYFPANYWGQGTQVTVSS.

In various embodiments of the aspects described herein, the VHH domain comprises a sequence having at least 75%, 80%, 85%, 90%, 95%, 96%, 97%, 98%, 99%, or 100% sequence identity with the sequence of QVQLVESGGGLVQAGGSLKLACAAPGLTFSSYRMGWFRQAPGQEREFVAAIDWNGRG TYYRYYADSVKGRSTISRDNAKNTMYLQMNSLKPEDTAVYYCAAGSIDLNWYGGMD YWGQGTQVTVSS (SEQ ID NO: 93). In various embodiments of the aspects described herein, the VHH domain comprises a framework comprising sequence having at least 75%, 80%, 85%, 90%, 95%, 96%, 97%, 98%, 99%, or 100% sequence identity with the sequence of EVQVVESGGGLVQAGGSLKLACAAPGLTFSSYRMGWFRQAPGQEREFVAAIDWNGRG TYYRYYADSVKGRSTISRDNAKNTVYLQMNSLKPEDTAVYYCAATTVLTDPRVLNEYA TWGQGTQVTVSS (SEQ ID NO: 94). In various embodiments of the aspects described herein, the VHH domain comprises a sequence having at least 75%, 80%, 85%, 90%, 95%, 96%, 97%, 98%, 99%, or 100% sequence identity with the sequence of QLQLVESGGGLVQPGGSLRLSCAASGSIFSINVMGWYRQAPGKQRELVARINGGGITHY AESVKGRFTISRDNAKNTVYLQMNSLKPEDTAAYYCKADVFGSSGYVETYWGQGTQV TVSS (SEQ ID NO: 95). In various embodiments of the aspects described herein, the VHH domain comprises a sequence having at least 75%, 80%, 85%, 90%, 95%, 96%, 97%, 98%, 99%, or 100% sequence identity with the sequence of EVQVVESGGGLVQAGGSLRLSCAVSGTSVSSNAMGWYRQAPGKQREWVGFIDRIATTT IATSVKGRFAITRDNAKNTVYLQMSGLKPEDTAVYYCNHPLTARWGQGTQVTVSS (SEQ ID NO: 96). In various embodiments of the aspects described herein, the VHH domain comprises a sequence having at least 75%, 80%, 85%, 90%, 95%, 96%, 97%, 98%, 99%, or 100% sequence identity with the sequence of QVQLVESGGGLVQAGGSLRLSCAASGRTFSSYAMGWFRQAPGKEREFVAAITWNGGTT YYADSVKGRFTISRDNAKNTVYLQMNSLKPEDTAVYYCAADPFNQGYWGQGTQVTVS S (SEQ ID NO: 97). In various embodiments of the aspects described herein, the VHH domain comprises a sequence having at least 75%, 80%, 85%, 90%, 95%, 96%, 97%, 98%, 99%, or 100% sequence identity with the sequence of EVQLVESGGGLVQAGGSLRLSCAVSGSSVSSDAMGWYRQAPGNQRAWVAFISGGGTT TYADSVKGRFTISRDNTKNTVYLHMNSLKPEDTAVYYCNHPLTSRWGQGTQVTVSS (SEQ ID NO: 98). In various embodiments of the aspects described herein, the VHH domain comprises a sequence having at least 75%, 80%, 85%, 90%, 95%, 96%, 97%, 98%, 99%, or 100% sequence identity with the sequence of EVQVVESGGGLVQAGGSLRLACVASRSIGSINVMGWYRQAPGKQRDLVARITGGGSTH YAESVKGRFTISRDNAKNTVYLQMNSLEPEDTAVYYCASMVNPIITAWGTIGVREIPDY DYWGQGTQVTVSS (SEQ ID NO: 99). In various embodiments of the aspects described herein, the VHH domain comprises a sequence having at least 75%, 80%, 85%, 90%, 95%, 96%, 97%, 98%, 99%, or 100% sequence identity with the sequence of QVQLVESGGGLVQAGGSLRLSCAVSGRTFSTYRMGWFRQAPGKERSFVAAISWSGGST TYADPVKGRFTISRDNAKNTVYLRMNSLKPEDTAVYYCNDQRGYWGQGTLVTVSS (SEQ ID NO: 100). In various embodiments of the aspects described herein, the VHH domain comprises a sequence having at least 75%, 80%, 85%, 90%, 95%, 96%, 97%, 98%, 99%, or 100% sequence identity with the sequence of EVQVVESGGGLVQAGGSLRLSCAASGFTFTRYAMGWFRQAPGKERSFVAAISWSGSSA GYGDSVKGRFTISRDNAKNTLYLQMNSLKPEDTAVYYCAADPFNQGYWGQGTQVTVS S (SEQ ID NO: 101). In various embodiments of the aspects described herein, the VHH domain comprises a sequence having at least 75%, 80%, 85%, 90%, 95%, 96%, 97%, 98%, 99%, or 100% sequence identity with the sequence of EVQVVESGGGLVQAGGSLRLSCAASGRTFTTYRMGWFRQAPGKEREFVAAIRWSGGRT LYADSVKGRFTISRDNAKNTAYLQMNNLRPEDTAVYYCAADLAEYSGTYSSPADSPAG YDYWGQGTQVTVSS (SEQ ID NO: 102). In various embodiments of the aspects described herein, the VHH domain comprises a sequence having at least 75%, 80%, 85%, 90%, 95%, 96%, 97%, 98%, 99%, or 100% sequence identity with the sequence of

(SEQ ID NO: 103)
QVQLVETGGGLVQAGDSLRLSCAASGRTLSFNTYAMGWFRQAPGKEREF
VASITWNGGSTSYADSVKGRFTITRDNAKNTATLRMNSLQPDDTAVYYC
AAARYYVSGTYFPANYWGQGTQVTVSS.

In some embodiments, the VHH molecules may be used to deliver biologics or other compositions from blood to mucus (IV injection). In some embodiments, the VHH molecules may be used to increase the stability and PK of orally-delivered biologics or other compositions that function in mucosal tissues. In some embodiments, the VHH molecules may be used for increasing the stability and PK of orally-delivered biologics and also to transport the VHH molecules back to mucosal tissues, which are leaked into systemic circulation, in cases where epithelial barrier is compromised such as intestinal bowel disease.

In another aspect is provided a therapeutic molecule comprising any of the VHH domains described herein, and an agent, including, for example, a therapeutic agent or a conjugate of an agent (e.g., a bioconjugate). Exemplary agents include, but are not limited to, an antibody or fragment thereof, a peptide, a vaccine, a small molecule, a polynucleotide (e.g., a nucleic acid molecule), a radioisotope, a toxin, an enzyme, an anticoagulant, a hormone, a cytokine, an anti-inflammatory molecule, an RNAi, an mRNA, a self-replicating RNA, an antibiotic, or an antibody-antibiotic conjugate. In a specific embodiment, the agent is an antibiotic. Exemplary antibiotics include, but are not limited to, macrolide antibiotic, a fluoroquinolone, a tetracycline, amoxicillin, ceftriaxone, penicillin G, linezolid, moxifloxacin, and azithromycin. Exemplary radioisotopes include, but are not limited to, from ¹⁸F, ⁹⁹Tc, ¹¹¹In, ¹²³I, ²⁰¹Tl, ¹³³Xe, ¹¹C, ¹³N, ¹⁵O, ¹⁸F, ⁶²Cu, ⁶⁴Cu, ¹²⁴I, ⁷⁶Br, ⁸²Rb, ⁸⁹Zr and ⁶⁸Ga. In a specific embodiment, the agent is a peptide. Exemplary peptides include, but are not limited to, an octreotide (e.g. Mycapssa), insulin or a derivative thereof (e.g. Capsulin OAD, ORMD-0801, Tregopil, HDV Insulin, Oshadi Icp, Dance 501, Exubera, Afrezza, Oral-lyn, MSL001-PH-2-1, NanoCelle Insulin), an insulin-mimic peptide, a semaglutide (e.g. NN9924), a leuprolide (e.g. Ovarest), a glucagon-like peptide 1 (e.g. TTP273), a glucagon-like-peptide-1-mimic peptides, an IL-23 receptor antagonist peptide (e.g., PTG-200), a salmon calcitonin (e.g. TBRIA), a desmopressin (e.g. DDAVP), a calcitonin (e.g. Miacalcin), an oxytocin (e.g. Syntocinon), and a nafarelin (e.g. Synarel). In a specific embodiment, the agent is a vaccine. Exemplary vaccines are useful for preventing inventions, including infections from Vibrio, Cholera, Typhoid, Rotavirus, Tuberculosis, HIV, Flu, Ebola, and Sendai. Exemplary vaccines include, but are not limited to, connaught strain BCG (e.g. BCG vaccine), a live attenuated cholera vaccine (e.g. Vaxchora), a live attenuated Salmonella enterica subsp. enterica seravar typi Ty21a (e.g. Vivotif), a live, noncovalent, human attenuated rotavirus strain (e.g. Rotarix), a live pentavalent bovine attenuated rotavirus strain (e.g. RotaTeq), a recombinant modified vaccinia virus Ankara expressing antigen 85A (MVA85A) (e.g. MVA85A), a live attenuated Bordetella pertussis (e.g. BPZE1), a flu vaccine (e.g. PUR003, INFLUSOME-VAC, FluMist Quadrivalent), a Tuberculosis vaccine (e.g. Ad5Ag85 Å, Tuberculosis vaccine), an HIV vaccine (e.g. EuroNeut41, HIV vaccine), an inactivated H5N1 influenza vaccine (e.g. GelVac), an RSVcps2 vaccine (e.g. Respiratory syncytial virus vaccine), a Shigellosis vaccine (e.g. Invaplex 50), an ebola vaccine (e.g. Ebola vaccine), and a Sendai vaccine (e.g. Sendai vaccine). In a specific embodiment, the agent is an antibody or fragment thereof. Exemplary antibodies or fragments thereof include, but are not limited to, an antitumour necrosis factor antibody (e.g. AVX-470), an anti-TNF-alpha antibody (e.g., infliximab), an anti-IL23 antibody (e.g., guselkumab), an antibody that binds to a receptor of IL23, an anti-IL12 and anti-IL23 antibody (e.g., uspekinumab), muromonab (e.g. OKT3), a homeopathic antibody (e.g. TAO1), an anti-CD3 antibody (e.g. aCD3, TZLS-401), and an immunoglobulin Y egg yolk antibody (e.g. AGY). In a specific embodiment, the agent is a cytokine. Exemplary cytokines include, but are not limited to, interferon-α. In a specific embodiment, the agent is a hormone. Exemplary hormones include, but are not limited to, desmopressin (e.g. DDAVP). In a specific embodiment, the agent is a small molecule. Exemplary small molecules include, but are not limited to, cyclosporin A (e.g. Neoral).

In another aspect, a therapeutic molecule comprising any of the VHH domains as described herein and an agent as described herein may be used for (e.g., diagnosis, prevention, and/or treatment) one or more of the following applications, indications, diseases, disorders or conditions including, but not limited to, an inflammatory disorder, a cardiovascular and metabolism (CVM) disorder, an intestinal bowel disease, inflammatory bowel disease, acromegaly, Type-1 diabetes, Type-2 diabetes, pharmacokinetic and pharmacodynamic profiles in healthy female volunteers, postmenopausal osteoporosis in women, tuberculosis vaccination, gut inflammation, ulcerative colitis, upper respiratory tract infections, hepatitis C, non-alcoholic steatohepatitis, coeliac disease, idiopathic pulmonary fibrosis, antidiuretic replacement therapy for diabetes insipidus, organ rejection prophylaxis, immunization against disease caused by Vibrio cholera serogroup O1, typhoid vaccination, prevention of rotavirus gastroenteritis, pharmacokinetic and pharmacodynamic profiles of Dance 501 in healthy subjects without diabetes but with mild to moderate asthma or COPD, Bordetella pertussis vaccination, flu vaccination, HIV vaccination, H5N1 influenza vaccination, respiratory syncytial virus cps2 vaccination, Shigellosis vaccination, Ebola vaccination, Sendai vaccination, antidiuretic replacement therapy for diabetes insipidus, symptomatic Paget disease, postmenopausal osteoporosis and fibromyalgia, induction of labour, central precocious puberty, embryo replacement therapy, endometriosis, pneumonia, cystic fibrosis, lung infection, asthma, tuberculosis, chronic obstructive pulmonary disease (COPD), bronchitis and emphysema, cystitis, overactive bladder disease, sinus infection, gastrointestinal ulcer, adenomyosis, uterine inflammation, hepatobiliary disease, or hepatitis.

Also provided is a zirconium labelled VHH-Fc conjugate. Zirconium-89 may be used. Other radioisotopes may be used instead of zirconium. The conjugate may be used for mucosal PET-CT imaging. The conjugate may be used to detect and diagnose lung cancer. Without wishing to be bound by theory, lung cancer originates from lung mucosa due to smoking, early diagnosis is beneficial. pIgR expression inversely correlates with lung cancer progression. The conjugate may also be used to detect and diagnose endometrial and colon cancer. pIgR overexpression can be common in endometrial and colon cancer. Detecting increased transport of the zirconium labelled VHH-Fc conjugate, as compared to a normal healthy subject, may be useful to diagnose endometrial and colon cancer. In addition, VHH domains coupled to therapeutic agents (e.g. therapeutic molecules) may be used to treat lung cancer, endometrial cancer, and colon cancer. The VHH domains can undergo increased transport to these tissues due to increased pIgR expression in lung cancer, endometrial cancer and colon cancer.

In various embodiments, the VHH domain is genetically fused or chemically conjugated to the agent. Genetic fusion may be accomplished by placing a linker (e.g., a polypeptide) between the VHH domain and the agent. The linker may be a flexible linker comprising a sequence selected from the group consisting of EPKTPKPQPQPQLQPQPNPTTESKSPK (SEQ ID NO: 130), (EAAAK)n (SEQ ID NO: 147), (GGGGS)n (SEQ ID NO: 148) and (GGGS)n (SEQ ID NO: 149), wherein n is an integer from 1 to 20. The VHH domain may be chemically-conjugated to the agent, or otherwise non-covalently conjugated to the agent.

In various embodiments, the VHH domain is genetically conjugated to a therapeutic molecule, with a hinge region linking the VHH domain to the therapeutic molecule. The hinge region may be a flexible linker comprising a sequence selected from the group consisting of EPKTPKPQPQPQLQPQPNPTTESKSPK (SEQ ID NO: 130), (EAAAK)n (SEQ ID NO: 147), (GGGGS)n (SEQ ID NO: 148) and (GGGS)n (SEQ ID NO: 149), wherein n is an integer from 1 to 20. In some embodiments, the hinge region comprises the sequence EPKTPKPQPQPQLQPQPNPTTESKSPK (SEQ ID NO: 130), or comprises an amino acid sequence having at least 50, at least 55, at least 60, at least 65, at least 70, at least 75, at least 80, at least 85, at least 90, at least 95, at least 98 or at least 99%, sequence identity with EPKTPKPQPQPQLQPQPNPTTESKSPK (SEQ ID NO: 130). In some embodiments, the hinge region comprises the sequence EPKSCDKTHTCPPCP (SEQ ID NO: 150), or comprises an amino acid sequence having at least 50, at least 55, at least 60, at least 65, at least 70, at least 75, at least 80, at least 85, at least 90, at least 95, at least 98 or at least 99%, sequence identity with EPKSCDKTHTCPPCP (SEQ ID NO: 150). In some embodiments, the hinge region comprises the sequence ERKCCVECPPCP (SEQ ID NO: 151), or comprises an amino acid sequence having at least 50, at least 55, at least 60, at least 65, at least 70, at least 75, at least 80, at least 85, at least 90, at least 95, at least 98 or at least 99%, sequence identity with ERKCCVECPPCP (SEQ ID NO: 151). In some embodiments, the hinge region comprises the sequence ELKTPLGDTTHTCPRCP(EPKSCDTPPPCPRCP)₃ (SEQ ID NO: 152), or comprises an amino acid sequence having at least 50, at least 55, at least 60, at least 65, at least 70, at least 75, at least 80, at least 85, at least 90, at least 95, at least 98 or at least 99%, sequence identity with ELKTPLGDTTHTCPRCP(EPKSCDTPPPCPRCP)₃ (SEQ ID NO: 152). In some embodiments, the hinge region comprises the sequence ESKYGPPCPSCP (SEQ ID NO: 153), or comprises an amino acid sequence having at least 50, at least 55, at least 60, at least 65, at least 70, at least 75, at least 80, at least 85, at least 90, at least 95, at least 98 or at least 99%, sequence identity with ESKYGPPCPSCP (SEQ ID NO: 153).

The agent may be a cytokine. The agent may be an anti-inflammatory molecule. The agent may be an antibody conjugated to an antibiotic. VHH domains coupled to cytokines may be used to treat a disease of the lung, whereby the cytokine is transported to the lung via interaction of the VHH domain with pIgR. VHH domains coupled to anti-inflammatory molecules may be used to treat a disease of the lung, whereby the anti-inflammatory molecule is transported to the lung via interaction of the VHH domain with pIgR. VHH domains coupled to antibiotics, or antibody-antibiotic conjugates, may be used to treat a lung infection, whereby the antibiotic or antibody-antibiotic conjugate is transported to the lung via interaction of the VHH domain with pIgR.

In another aspect is provided a nucleic acid molecule encoding any of the VHH domains described herein. In exemplary embodiments, the nucleic acid molecule encodes the VHH domain having the sequence of:

(SEQ ID NO: 93)
QVQLVESGGGLVQAGGSLKLACAAPGLTFSSYRMGWFRQAPGQEREFVA
AIDWNGRGTYYRYYADSVKGRSTISRDNAKNTMYLQMNSLKPEDTAVYY
CAAGSIDLNWYGGMDYWGQGTQVTVSS,
(SEQ ID NO: 94)
EVQVVESGGGLVQAGGSLKLACAAPGLTFSSYRMGWFRQAPGQEREFVA
AIDWNGRGTYYRYYADSVKGRSTISRDNAKNTVYLQMNSLKPEDTAVYY
CAATTVLTDPRVLNEYATWGQGTQVTVSS,
(SEQ ID NO: 95)
QLQLVESGGGLVQPGGSLRLSCAASGSIFSINVMGWYRQAPGKQRELVA
RINGGGITHYAESVKGRFTISRDNAKNTVYLQMNSLKPEDTAAYYCKAD
VFGSSGYVETYWGQGTQVTVSS,
(SEQ ID NO: 96)
EVQVVESGGGLVQAGGSLRLSCAVSGTSVSSNAMGWYRQAPGKQREWVG
FIDRIATTTIATSVKGRFAITRDNAKNTVYLQMSGLKPEDTAVYYCNHP
LTARWGQGTQVTVSS,
(SEQ ID NO: 97)
QVQLVESGGGLVQAGGSLRLSCAASGRTFSSYAMGWFRQAPGKEREFVA
AITWNGGTTYYADSVKGRFTISRDNAKNTVYLQMNSLKPEDTAVYYCAA
DPFNQGYWGQGTQVTVSS,
(SEQ ID NO: 98)
EVQLVESGGGLVQAGGSLRLSCAVSGSSVSSDAMGWYRQAPGNQRAWVA
FISGGGTTTYADSVKGRFTISRDNTKNTVYLHMNSLKPEDTAVYYCNHP
LTSRWGQGTQVTVSS,
(SEQ ID NO: 99)
EVQVVESGGGLVQAGGSLRLACVASRSIGSINVMGWYRQAPGKQRDLVA
RITGGGSTHYAESVKGRFTISRDNAKNTVYLQMNSLEPEDTAVYYCASM
VNPIITAWGTIGVREIPDYDYWGQGTQVTVSS,
(SEQ ID NO: 100)
QVQLVESGGGLVQAGGSLRLSCAVSGRTFSTYRMGWFRQAPGKERSFVA
AISWSGGSTTYADPVKGRFTISRDNAKNTVYLRMNSLKPEDTAVYYCND
QRGYWGQGTLVTVSS,
(SEQ ID NO: 101)
EVQVVESGGGLVQAGGSLRLSCAASGFTFTRYAMGWFRQAPGKERSFVA
AISWSGSSAGYGDSVKGRFTISRDNAKNTLYLQMNSLKPEDTAVYYCAA
DPFNQGYWGQGTQVTVSS,
(SEQ ID NO: 102)
EVQVVESGGGLVQAGGSLRLSCAASGRTFTTYRMGWFRQAPGKEREFVA
AIRWSGGRTLYADSVKGRFTISRDNAKNTAYLQMNNLRPEDTAVYYCAA
DLAEYSGTYSSPADSPAGYDYWGQGTQVTVSS,
or
(SEQ ID NO: 103)
QVQLVETGGGLVQAGDSLRLSCAASGRTLSFNTYAMGWFRQAPGKEREF
VASITWNGGSTSYADSVKGRFTITRDNAKNTATLRMNSLQPDDTAVYYC
AAARYYVSGTYFPANYWGQGTQVTVSS.

In exemplary embodiments, the nucleic acid molecule comprises the sequence of:

(SEQ ID NO: 133)
Caggtgcagctggtggagtctgggggaggattggtgcaggctgggggctc
tctgaaactcgcctgtgcagcacctggacttaccttcagttcgtatcgca
tgggctggttccgccaggctccagggcaggagcgtgagtttgtagcagct
attgattggaatggtcgtggcacatattatcgatactatgcagactccgt
gaagggccgatccaccatttccagagacaacgccaagaacacgatgtatc
tgcaaatgaacagcctgaaacctgaggacacggccgtttattactgtgca
gcaggttcgatcgaccttaactggtacggcggcatggactactggggcca
ggggacccagcgtctcctca,
(SEQ ID NO: 134)
gaggtgcaggtggtggagtctgggggaggattggtgcaggctgggggctc
tctgaaactcgcctgtgcagcacctggacttaccttcagttcgtatcgca
tgggctggttccgccaggctccagggcaggagcgtgagtttgtagcagct
attgattggaatggtcgtggcacatattatcgatactatgcagactccgt
gaagggccgatccaccatttccagagacaacgccaagaacacggtgtatc
tgcaaatgaacagcctgaaacctgaggacacggccgtttattactgtgca
gctactacggtattaactgaccctagggttcttaatgagtatgccacatg
gggccaggggacccaggtcaccgtctcctca,
(SEQ ID NO: 135)
cagttgcagctcgtggagtctgggggaggcttggtgcagcctggggggtc
tctgagactctcctgtgcagcctctggaagcatcttcagtatcaatgtta
tgggctggtaccgccaggctccagggaagcagcgcgagttggtcgcacgt
attaatggaggtggcattacacactatgcagagtccgtgaagggccgatt
caccatctccagagacaacgccaagaacacggtgtatctgcaaatgaaca
gcctgaaacctgaggacacagccgcatattactgtaaggcagatgtgttc
ggtagtagcgggtacgtagaaacctactggggccaggggacccaggtcac
cgtctcctca,
(SEQ ID NO: 136)
Gaggtgcaggtggtggagtctgggggaggcttggtgcaggctgggggctc
tctgagactctcctgtgcagtctctggaacctccgtcagtagcaatgcca
tgggttggtaccgccaggctccagggaagcagcgcgagtgggtcggattt
attgatcgtattgctaccacgacgattgcaacctccgtgaagggccgatt
cgccatcaccagagacaacgccaagaacacggtgtatctccaaatgagcg
gcctgaaacctgaggacacagccgtctattactgtaatcatccattgacc
gctcggtggggccaggggacccaggtcaccgtctcctca,
(SEQ ID NO: 137)
Caggtgcagctggtggagtctgggggaggcttggtgcaggctgggggctc
tctgagactctcctgtgcagcctctggacgcaccttcagtagctatgcca
tgggctggttccgccaggctccagggaaggagcgtgagtttgtagcagct
attacctggaatggtggtaccacatactatgcagactccgtgaagggccg
attcaccatctccagagacaacgccaagaacacggtgtatctgcaaatga
acagcctgaaacctgaggacacggccgtttattactgtgcagcagaccca
ttcaaccaaggctactggggccaggggacccaggtcaccgtctcctca,
(SEQ ID NO: 138)
Gaggtgcagctcgtggagtctggaggaggcttggtgcaggctggggggtc
tctgagactctcctgtgcagtctctggaagctccgtcagtagcgatgcca
tgggttggtaccgccaggctccagggaatcagcgcgcgtgggtcgcattt
atttctggtggtggtaccacaacctatgcagactccgttaagggccgatt
caccatctccagagacaacaccaagaacacggtgtatctccacatgaaca
gcctgaaacctgaagacacagccgtctattactgtaatcatccattgacg
tctcggtggggccaggggacccaggtcaccgtctcctca,
(SEQ ID NO: 139)
Gaggtgcaggtggtggagtctgggggaggattggtgcaggctggggggtc
tctgagactcgcctgtgtagcctctagaagcatcggcagtatcaatgtta
tgggctggtaccgccaggctccagggaagcagcgcgacttggtcgcacgt
attactggaggtggcagtacacactacgcagagtccgtgaagggccgatt
caccatctccagagacaacgccaagaacacggtgtatctgcaaatgaaca
gcctggaacctgaggacacggccgtttattactgtgcgtcaatggtaaac
cctatcattacggcttggggtacgattggtgtgcgcgagattcccgacta
tgactactggggccaggggacccaggtcaccgtctcctca,
(SEQ ID NO: 140)
Gaggtgcaggtggtggagtctgggggaggcttggtgcaggctggggggtc
tctgagactctcctgtgcagcctctggattcaccttcacccgctatgcca
tgggctggttccgccaggctccagggaaggagcgatcgtttgtagcagct
attagctggagtggtagtagcgcaggctatggagactccgtgaagggccg
attcaccatctccagagacaacgccaagaacacgctgtatctgcaaatga
acagtctaaaacctgaggacacggccgtttattactgtgcagcagaccca
ttcaaccaaggctactggggccaggggacccaggtcaccgtctcctca,
(SEQ ID NO: 141)
Gaggtgcaggtggtggagtctgggggaggattggtgcaggctgggggctc
tctgagactctcctgtgcagcctctggacgcaccttcactacctatcgca
tgggctggttccgccaggctccagggaaggagcgagagtttgtagcagct
attcgctggagtggtggtcgcacattgtatgcagactccgtgaagggccg
attcaccatctccagagacaacgccaagaacacagcgtatctgcaaatga
acaacctgagacctgaggacacggccgtttattactgtgcagcagatcta
gccgagtatagtggtacttactccagccctgcggactcccccgctgggta
tgactactggggccaggggacccaggtcaccgtctcctca,
or
(SEQ ID NO: 142)
caggtgcagctggtcgaaactgggggaggattggtgcaggctggggactc
tctgagactctcctgtgcagcctctggacgcaccctcagcttcaacacct
atgccatgggctggttccgccaggctccagggaaggagcgtgaatttgta
gcctctattacctggaatggtggaagcacaagctacgcagactccgtgaa
gggccgattcaccatcaccagagacaacgccaagaacacggctactctgc
gaatgaatagcctgcagcccgacgacacggccgtgtattactgtgcagca
gcccgatactatgtgagtggtacttacttccccgcgaattactggggcca
ggggacccaggtcaccgtctcctca.

Also provided are vectors comprising the nucleic acid molecules described herein. In an embodiment, the nucleic acid molecules can be incorporated into a recombinant expression vector. The present disclosure provides recombinant expression vectors comprising any of the nucleic acids of the invention. As used herein, the term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The vectors described herein are not naturally-occurring as a whole; however, parts of the vectors can be naturally-occurring. The described recombinant expression vectors can comprise any type of nucleotides, including, but not limited to DNA and RNA, which can be single-stranded or double-stranded, synthesized or obtained in part from natural sources, and which can contain natural, non-natural or altered nucleotides. The recombinant expression vectors can comprise naturally-occurring or non-naturally-occurring internucleotide linkages, or both types of linkages. The non-naturally occurring or altered nucleotides or internucleotide linkages do not hinder the transcription or replication of the vector.

In an embodiment, the recombinant expression vector of the invention can be any suitable recombinant expression vector, and can be used to transform or transfect any suitable host. Suitable vectors include those designed for propagation and expansion or for expression or both, such as plasmids and viruses. The vector can be selected from the group consisting of the pUC series (Fermentas Life Sciences, Glen Burnie, Md.), the pBluescript series (Stratagene, LaJolla, Calif.), the pET series (Novagen, Madison, Wis.), the pGEX series (Pharmacia Biotech, Uppsala, Sweden), and the pEX series (Clontech, Palo Alto, Calif.). Bacteriophage vectors, such as λGT10, λGT11, λEMBL4, and λNM1149, λZapII (Stratagene) can be used. Examples of plant expression vectors include pBI01, pBI01.2, pBI121, pBI101.3, and pBIN19 (Clontech). Examples of animal expression vectors include pEUK-Cl, pMAM, and pMAMneo (Clontech). The recombinant expression vector may be a viral vector, e.g., a retroviral vector, e.g., a gamma retroviral vector.

In an embodiment, the recombinant expression vectors are prepared using standard recombinant DNA techniques described in, for example, Sambrook et al., supra, and Ausubel et al., supra. Constructs of expression vectors, which are circular or linear, can be prepared to contain a replication system functional in a prokaryotic or eukaryotic host cell. Replication systems can be derived, e.g., from ColE1, SV40, 2μ plasmid, λ, bovine papilloma virus, and the like.

The recombinant expression vector may comprise regulatory sequences, such as transcription and translation initiation and termination codons, which are specific to the type of host (e.g., bacterium, plant, fungus, or animal) into which the vector is to be introduced, as appropriate, and taking into consideration whether the vector is DNA- or RNA-based.

The recombinant expression vector can include one or more marker genes, which allow for selection of transformed or transfected hosts. Marker genes include biocide resistance, e.g., resistance to antibiotics, heavy metals, etc., complementation in an auxotrophic host to provide prototrophy, and the like. Suitable marker genes for the described expression vectors include, for instance, neomycin/G418 resistance genes, histidinol x resistance genes, histidinol resistance genes, tetracycline resistance genes, and ampicillin resistance genes.

The recombinant expression vector can comprise a native or normative promoter operably linked to the nucleic acid molecules described herein. The selection of promoters, e.g., strong, weak, tissue-specific, inducible and developmental-specific, is within the ordinary skill of the artisan. Similarly, the combining of a nucleotide sequence with a promoter is also within the skill of the artisan. The promoter can be a non-viral promoter or a viral promoter, e.g., a cytomegalovirus (CMV) promoter, an RSV promoter, an SV40 promoter, or a promoter found in the long-terminal repeat of the murine stem cell virus.

The recombinant expression vectors can be designed for either transient expression, for stable expression, or for both. Also, the recombinant expression vectors can be made for constitutive expression or for inducible expression.

Further, the recombinant expression vectors can be made to include a suicide gene. As used herein, the term “suicide gene” refers to a gene that causes the cell expressing the suicide gene to die. The suicide gene can be a gene that confers sensitivity to an agent, e.g., a drug, upon the cell in which the gene is expressed, and causes the cell to die when the cell is contacted with or exposed to the agent. Suicide genes are known in the art and include, for example, the Herpes Simplex Virus (HSV) thymidine kinase (TK) gene, cytosine deaminase, purine nucleoside phosphorylase, and nitroreductase.

Included in the scope of the invention are conjugates, e.g., bioconjugates, comprising any of polypeptides, or proteins (including any of the functional portions or variants thereof), host cells, nucleic acids, recombinant expression vectors, populations of host cells, or antibodies, or antigen binding portions thereof. Conjugates, as well as methods of synthesizing conjugates in general, are known in the art (See, for instance, Hudecz, F., Methods Mol. Biol. 298: 209-223 (2005) and Kirin et al., Inorg Chem. 44(15): 5405-5415 (2005)).

Also provided are host cells comprising the nucleic acid molecules described herein. The host cell may be any cell that contains a heterologous nucleic acid. The heterologous nucleic acid can be a vector (e.g., an expression vector). For example, a host cell can be a cell from any organism that is selected, modified, transformed, grown, used or manipulated in any way, for the production of a substance by the cell, for example the expression by the cell of a gene, a DNA or RNA sequence, a protein or an enzyme. An appropriate host may be determined. For example, the host cell may be selected based on the vector backbone and the desired result. By way of example, a plasmid or cosmid can be introduced into a prokaryote host cell for replication of several types of vectors. Bacterial cells such as, but not limited to DH5α, JM109, and KCB, SURE® Competent Cells, and SOLOPACK Gold Cells, can be used as host cells for vector replication and/or expression. Additionally, bacterial cells such as E. coli LE392 could be used as host cells for phage viruses. Eukaryotic cells that can be used as host cells include, but are not limited to yeast (e.g., YPH499, YPH500 and YPH501), insects and mammals. Examples of mammalian eukaryotic host cells for replication and/or expression of a vector include, but are not limited to, HeLa, NIH3T3, Jurkat, 293, COS, Saos, PC12, SP2/0 (American Type Culture Collection (ATCC), Manassas, Va., CRL-1581), NS0 (European Collection of Cell Cultures (ECACC), Salisbury, Wiltshire, UK, ECACC No. 85110503), FO (ATCC CRL-1646) and Ag653 (ATCC CRL-1580) murine cell lines. An exemplary human myeloma cell line is U266 (ATTC CRL-TIB-196). Other useful cell lines include those derived from Chinese Hamster Ovary (CHO) cells such as CHO-K1SV (Lonza Biologics, Walkersville, Md.), CHO-K1 (ATCC CRL-61) or DG44.

Also provided are pharmaceutical compositions comprising any VHH domain described herein, including, for example, a VHH domain and an agent, such as a therapeutic molecule, comprising any of the VHH domains as described herein and an agent, and a pharmaceutically acceptable carrier (e.g., diluent, or excipient). In some embodiments, the pharmaceutical composition comprises an effective amount of any VHH domain described herein.

A pharmaceutically acceptable carrier can be an ingredient in a pharmaceutical composition, other than an active ingredient, which is nontoxic to the subject. A pharmaceutically acceptable carrier can include, but is not limited to, a buffer, excipient, stabilizer, or preservative. Examples of pharmaceutically acceptable carriers are solvents, dispersion media, coatings, antibacterial and antifungal agents, isotonic and absorption delaying agents, and the like that are physiologically compatible, such as salts, buffers, antioxidants, saccharides, aqueous or non-aqueous carriers, preservatives, wetting agents, surfactants or emulsifying agents, or combinations thereof. The amounts of pharmaceutically acceptable carrier(s) in the pharmaceutical compositions may be determined experimentally based on the activities of the carrier(s) and the desired characteristics of the formulation, such as stability and/or minimal oxidation.

Such compositions may comprise buffers such as acetic acid, citric acid, formic acid, succinic acid, phosphoric acid, carbonic acid, malic acid, aspartic acid, histidine, boric acid, Tris buffers, HEPPSO, HEPES, neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); antibacterial and antifungal agents; and preservatives.

Compositions of the present disclosure can be formulated for a variety of means of parenteral or non-parenteral administration. In one embodiment, the compositions can be formulated for infusion or intravenous administration. Compositions disclosed herein can be provided, for example, as sterile liquid preparations, e.g., isotonic aqueous solutions, emulsions, suspensions, dispersions, or viscous compositions, which may be buffered to a desirable pH. Formulations suitable for oral administration can include liquid solutions, capsules, sachets, tablets, lozenges, and troches, powders liquid suspensions in an appropriate liquid and emulsions.

The term “pharmaceutically acceptable,” as used herein with regard to pharmaceutical compositions, means approved by a regulatory agency of the Federal or a state government or listed in the U.S. Pharmacopeia or other generally recognized pharmacopeia for use in animals and/or in humans.

In another aspect is provided a method of increasing the rate of pIgR-mediated transcytosis (e.g., forward transcytosis and/or reverse transcytosis) across an epithelial cell, including, for example, as measured by any assays or models of forward transcytosis and/or reverse transcytosis as described herein. The method comprises contacting the cell with any VHH domain or therapeutic molecule comprising the VHH domain described herein. In some embodiments, the method does not inhibit pIgR-mediated transcytosis of IgA. The VHH domain or the therapeutic molecule may comprise a CDR1 sequence of SNAMG (SEQ ID NO: 3), INVMG (SEQ ID NO: 6), TYRMG (SEQ ID NO: 7), RYAMG (SEQ ID NO: 8), FTTYRMG (SEQ ID NO: 258), TYRMG (SEQ ID NO: 259), FNTYAMG (SEQ ID NO: 9), GTSVSSN (SEQ ID NO: 12), GRTFSSY (SEQ ID NO: 13), RSIGSIN (SEQ ID NO: 15), GRTFSTY (SEQ ID NO: 16), GFTFTRY (SEQ ID NO: 17), GRTFTTY (SEQ ID NO: 18), GRTLSFNTY (SEQ ID NO: 19), GTSVSSNA (SEQ ID NO: 22), RSIGSINV (SEQ ID NO: 25), GRTFSTYR (SEQ ID NO: 26), GFTFTRYA (SEQ ID NO: 27), GRTFTTYR (SEQ ID NO: 28), GRTLSFNTYA (SEQ ID NO: 29), GTSVSSNAMG (SEQ ID NO: 156), RSIGSINVMG (SEQ ID NO: 159), GRTFSTYRMG (SEQ ID NO: 160), GFTFTRYAMG (SEQ ID NO: 161), GRTFTTYRMG (SEQ ID NO: 162), GRTLSFNTYAMG (SEQ ID NO: 163), SSNAMG (SEQ ID NO: 166), SINVMG (SEQ ID NO: 169), STYRMG (SEQ ID NO: 170), TRYAMG (SEQ ID NO: 171), TTYRMG (SEQ ID NO: 172), SFNTYAMG (SEQ ID NO: 173), GTSVSSNAMG (SEQ ID NO: 176), RSIGSINVMG (SEQ ID NO: 179), GRTFSTYRMG (SEQ ID NO: 180), GFTFTRYAMG (SEQ ID NO: 181), GRTFTTYRMG (SEQ ID NO: 182), or GRTLSFNTYAMG (SEQ ID NO: 183). The VHH domain or the therapeutic molecule may comprise a CDR2 sequence of FIDRIATTTIATSVKG (SEQ ID NO: 32), RITGGGSTHYAESVKG (SEQ ID NO: 35), AISWSGGSTTYADPVKG (SEQ ID NO: 36), AISWSGSSAGYGDSVKG (SEQ ID NO: 37), AIRWSGGRTLYADSVKG (SEQ ID NO: 38), SITWNGGSTSYADSVKG (SEQ ID NO: 39), DRIAT (SEQ ID NO: 42), RIA (SEQ ID NO: 262), TGGGS (SEQ ID NO: 45), GGG (SEQ ID NO: 265), SWSGGS (SEQ ID NO: 46), WSGG (SEQ ID NO: 266), SWSGSS (SEQ ID NO: 47), WSGS (SEQ ID NO: 267), RWSGGR (SEQ ID NO: 48), WSGG (SEQ ID NO: 268), TWNGGS (SEQ ID NO: 49), WNGG (SEQ ID NO: 269), IDRIATT (SEQ ID NO: 52), ITGGGST (SEQ ID NO: 55), ISWSGGST (SEQ ID NO: 56), ISWSGSSA (SEQ ID NO: 57), IRWSGGRT (SEQ ID NO: 58), ITWNGGST (SEQ ID NO: 59), FIDRIATTTIATSVKG (SEQ ID NO: 186), RITGGGSTHYAESVKG (SEQ ID NO: 189), AISWSGGSTTYADPVKG (SEQ ID NO: 190), AISWSGSSAGYGDSVKG (SEQ ID NO: 191), AIRWSGGRTLYADSVKG (SEQ ID NO: 192), SITWNGGSTSYADSVKG (SEQ ID NO: 193), WVGFIDRIATTT (SEQ ID NO: 196), LVARITGGGSTH (SEQ ID NO: 199), FVAAISWSGGSTT (SEQ ID NO: 200), FVAAISWSGSSAG (SEQ ID NO: 201), FVAAIRWSGGRTL (SEQ ID NO: 202), FVASITWNGGSTS (SEQ ID NO: 203), FIDRIATTT (SEQ ID NO: 206), RITGGGSTH (SEQ ID NO: 209), AISWSGGSTT (SEQ ID NO: 210), AISWSGSSAG (SEQ ID NO: 211), AIRWSGGRTL (SEQ ID NO: 212), or SITWNGGSTS (SEQ ID NO: 213). The VHH domain or the therapeutic molecule may comprise a CDR3 sequence of PLTAR (SEQ ID NO: 63), MVNPIITAWGTIGVREIPDYDY (SEQ ID NO: 66), DQRGY (SEQ ID NO: 67), QRGY (SEQ ID NO: 271), DPFNQGY (SEQ ID NO: 68), DLAEYSGTYSSPADSPAGYDY (SEQ ID NO: 69), ARYYVSGTYFPANY (SEQ ID NO: 70), PLTAR (SEQ ID NO: 74), LTA (SEQ ID NO: 275), MVNPIITAWGTIGVREIPDYDY (SEQ ID NO: 77), VNPIITAWGTIGVREIPDYD (SEQ ID NO: 278), DQRGY (SEQ ID NO: 78), RG (SEQ ID NO: 279), DPFNQGY (SEQ ID NO: 79), PFNQG (SEQ ID NO: 280), DLAEYSGTYSSPADSPAGYDY (SEQ ID NO: 80), LAEYSGTYSSPADSPAGYD (SEQ ID NO: 281), ARYYVSGTYFPANY (SEQ ID NO: 81), RYYVSGTYFPAN (SEQ ID NO: 282), NHPLTAR (SEQ ID NO: 85), ASMVNPIITAWGTIGVREIPDYDY (SEQ ID NO: 88), NDQRGY (SEQ ID NO: 89), AADPFNQGY (SEQ ID NO: 90), AADLAEYSGTYSSPADSPAGYDY (SEQ ID NO: 91), AAARYYVSGTYFPANY (SEQ ID NO: 92), PLTAR (SEQ ID NO: 217), MVNPIITAWGTIGVREIPDYDY (SEQ ID NO: 220), QRGY (SEQ ID NO: 221), DPFNQGY (SEQ ID NO: 222), DLAEYSGTYSSPADSPAGYDY (SEQ ID NO: 223), ARYYVSGTYFPANY (SEQ ID NO: 224), NHPLTA (SEQ ID NO: 228), ASMVNPIITAWGTIGVREIPDYD (SEQ ID NO: 231), NDQRG (SEQ ID NO: 232), AADPFNQG (SEQ ID NO: 233), AADLAEYSGTYSSPADSPAGYD (SEQ ID NO: 234), AAARYYVSGTYFPAN (SEQ ID NO: 235), PLTAR (SEQ ID NO: 239), MVNPIITAWGTIGVREIPDYDY (SEQ ID NO: 242), QRGY (SEQ ID NO: 243), DPFNQGY (SEQ ID NO: 244), DLAEYSGTYSSPADSPAGYDY (SEQ ID NO: 245), or ARYYVSGTYFPANY (SEQ ID NO: 246).

In another aspect is provided a method of modulating a function of pIgR in a cell, including, for example, as measured by any assays or models of pIgR function as described herein. The method comprises contacting the cell with any VHH domain described herein, or any molecule comprising a VHH domain and an agent (e.g., therapeutic molecule) described herein. In some embodiments, modulation is activation of the function of pIgR. In some embodiments, modulation is inhibition of the function of pIgR. In some embodiments, the cell is a mucosal epithelial cell. In some embodiments, the cell is a cancer cell. Exemplary cancer cells include, but are not limited to, a lung cancer cell, an esophageal cancer cell, a stomach cancer cell, a duodenal cancer cell, a liver cancer cell, a bladder cancer cell, a sinus cancer cell, a nasal cavity cancer cell, an endometrial cancer cell or a colorectal cancer cell. The cell may be in a subject. The molecule (e.g., therapeutic molecule) may comprise an antibody or fragment thereof, a peptide, a vaccine, a small molecule, a polynucleotide, a molecule comprising a radioactive isotope, a toxin, an enzyme, an anticoagulant, a hormone, a cytokine, an anti-inflammatory molecule, an RNAi, an mRNA, a self-replicating RNA, an antibiotic, and an antibody-antibiotic conjugate as described herein. In certain embodiments, the molecule (e.g., therapeutic molecule) comprises an antibiotic (e.g., a macrolide antibiotic, a fluoroquinolone, a tetracycline, amoxicillin, ceftriaxone, penicillin G, linezolid, moxifloxacin, or azithromycin.) The molecule may be administered to the bloodstream of the subject. In a specific embodiment, the molecule (e.g., therapeutic molecule) comprises a peptide. Exemplary peptides include, but are not limited to, an octreotide (e.g. Mycapssa), insulin or a derivative thereof (e.g. Capsulin OAD, ORMD-0801, Tregopil, HDV Insulin, Oshadi Icp, Dance 501, Exubera, Afrezza, Oral-lyn, MSL001-PH-2-1, NanoCelle Insulin), an insulin-mimic peptide, a semaglutide (e.g. NN9924), a leuprolide (e.g. Ovarest), a glucagon-like peptide 1 (e.g. TTP273), a glucagon-like-peptide-1-mimic peptides, an IL-23 receptor antagonist peptide (e.g., PTG-200), a salmon calcitonin (e.g. TBRIA), a desmopressin (e.g. DDAVP), a calcitonin (e.g. Miacalcin), an oxytocin (e.g. Syntocinon), and a nafarelin (e.g. Synarel). In a specific embodiment, the molecule (e.g., therapeutic molecule) comprises a vaccine. Exemplary vaccines are useful for preventing inventions, including infections from Vibrio, Cholera, Typhoid, Rotavirus, Tuberculosis, HIV, Flu, Ebola, and Sendai. Exemplary vaccines include, but are not limited to, connaught strain BCG (e.g. BCG vaccine), a live attenuated cholera vaccine (e.g. Vaxchora), a live attenuated Salmonella enterica subsp. enterica seravar typi Ty21a (e.g. Vivotif), a live, noncovalent, human attenuated rotavirus strain (e.g. Rotarix), a live pentavalent bovine attenuated rotavirus strain (e.g. RotaTeq), a recombinant modified vaccinia virus Ankara expressing antigen 85A (MVA85A) (e.g. MVA85A), a live attenuated Bordetella pertussis (e.g. BPZE1), a flu vaccine (e.g. PUR003, INFLUSOME-VAC, FluMist Quadrivalent), a Tuberculosis vaccine (e.g. Ad5Ag85 Å, Tuberculosis vaccine), an HIV vaccine (e.g. EuroNeut41, HIV vaccine), an inactivated H5N1 influenza vaccine (e.g. GelVac), an RSVcps2 vaccine (e.g. Respiratory syncytial virus vaccine), a Shigellosis vaccine (e.g. Invaplex 50), an ebola vaccine (e.g. Ebola vaccine), and a Sendai vaccine (e.g. Sendai vaccine). In a specific embodiment, the molecule (e.g., therapeutic molecule) comprises an antibody or fragment thereof. Exemplary antibodies or fragments thereof include, but are not limited to, an antitumour necrosis factor antibody (e.g. AVX-470), an anti-TNF-alpha antibody (e.g., infliximab), an anti-IL23 antibody (e.g., guselkumab), an antibody that binds to a receptor of IL23, an anti-IL12 and anti-IL23 antibody (e.g., uspekinumab), muromonab (e.g. OKT3), a homeopathic antibody (e.g. TAO1), an anti-CD3 antibody (e.g. aCD3, TZLS-401), and an immunoglobulin Y egg yolk antibody (e.g. AGY). In a specific embodiment, the agent is a cytokine. Exemplary cytokines include, but are not limited to, interferon-α. In a specific embodiment, the molecule (e.g., therapeutic molecule) comprises is a hormone. Exemplary hormones include, but are not limited to, desmopressin (e.g. DDAVP). In a specific embodiment, the molecule (e.g., therapeutic molecule) comprises a small molecule. Exemplary small molecules include, but are not limited to, cyclosporin A (e.g. Neoral). The molecule may be administered intravenously or subcutaneously. The molecule may be administered by oral delivery, buccal delivery, nasal delivery or inhalation delivery, including for delivery to systemic circulation or Lamina propria.

In another aspect is provided a method of delivering a molecule (e.g., a therapeutic molecule) to a pIgR-expressing cell, including, for example, as measured by any assays or models of delivery as described herein. The method comprises contacting the cell with any VHH domain and an agent (e.g., therapeutic molecule) described herein, or any molecule comprising a VHH domain and an agent (e.g., therapeutic molecule) described herein. In some embodiments, the cell is a mucosal epithelial cell. In some embodiments, the cell is a cancer cell. Exemplary cancer cells include, but are not limited to, a lung cancer cell, an esophageal cancer cell, a stomach cancer cell, a duodenal cancer cell, a liver cancer cell, a bladder cancer cell, a sinus cancer cell, a nasal cavity cancer cell, an endometrial cancer cell or a colorectal cancer cell. The cell may be in a subject. The molecule (e.g., therapeutic molecule) may comprise an antibody or fragment thereof, a peptide, a vaccine, a small molecule, a polynucleotide, a molecule comprising a radioactive isotope, a toxin, an enzyme, an anticoagulant, a hormone, a cytokine, an anti-inflammatory molecule, an RNAi, an mRNA, a self-replicating RNA, an antibiotic, and an antibody-antibiotic conjugate as described herein. In certain embodiments, the molecule (e.g., therapeutic molecule) comprises an antibiotic (e.g., a macrolide antibiotic, a fluoroquinolone, a tetracycline, amoxicillin, ceftriaxone, penicillin G, linezolid, moxifloxacin, or azithromycin). In a specific embodiment, the molecule (e.g., therapeutic molecule) comprises a peptide. Exemplary peptides include, but are not limited to, an octreotide (e.g. Mycapssa), insulin or a derivative thereof (e.g. Capsulin OAD, ORMD-0801, Tregopil, HDV Insulin, Oshadi Icp, Dance 501, Exubera, Afrezza, Oral-lyn, MSL001-PH-2-1, NanoCelle Insulin), an insulin-mimic peptide, a semaglutide (e.g. NN9924), a leuprolide (e.g. Ovarest), a glucagon-like peptide 1 (e.g. TTP273), a glucagon-like-peptide-1-mimic peptides, an IL-23 receptor antagonist peptide (e.g., PTG-200), a salmon calcitonin (e.g. TBRIA), a desmopressin (e.g. DDAVP), a calcitonin (e.g. Miacalcin), an oxytocin (e.g. Syntocinon), and a nafarelin (e.g. Synarel). In a specific embodiment, the molecule (e.g., therapeutic molecule) comprises a vaccine. Exemplary vaccines are useful for preventing inventions, including infections from Vibrio, Cholera, Typhoid, Rotavirus, Tuberculosis, HIV, Flu, Ebola, and Sendai. Exemplary vaccines include, but are not limited to, connaught strain BCG (e.g. BCG vaccine), a live attenuated cholera vaccine (e.g. Vaxchora), a live attenuated Salmonella enterica subsp. enterica seravar typi Ty21a (e.g. Vivotif), a live, noncovalent, human attenuated rotavirus strain (e.g. Rotarix), a live pentavalent bovine attenuated rotavirus strain (e.g. RotaTeq), a recombinant modified vaccinia virus Ankara expressing antigen 85A (MVA85A) (e.g. MVA85A), a live attenuated Bordetella pertussis (e.g. BPZE1), a flu vaccine (e.g. PUR003, INFLUSOME-VAC, FluMist Quadrivalent), a Tuberculosis vaccine (e.g. Ad5Ag85 Å, Tuberculosis vaccine), an HIV vaccine (e.g. EuroNeut41, HIV vaccine), an inactivated H5N1 influenza vaccine (e.g. GelVac), an RSVcps2 vaccine (e.g. Respiratory syncytial virus vaccine), a Shigellosis vaccine (e.g. Invaplex 50), an ebola vaccine (e.g. Ebola vaccine), and a Sendai vaccine (e.g. Sendai vaccine). In a specific embodiment, the molecule (e.g., therapeutic molecule) comprises an antibody or fragment thereof. Exemplary antibodies or fragments thereof include, but are not limited to, an antitumour necrosis factor antibody (e.g. AVX-470), an anti-TNF-alpha antibody (e.g., infliximab), an anti-IL23 antibody (e.g., guselkumab), an antibody that binds to a receptor of IL23, an anti-IL12 and anti-IL23 antibody (e.g., uspekinumab), muromonab (e.g. OKT3), a homeopathic antibody (e.g. TAO1), an anti-CD3 antibody (e.g. aCD3, TZLS-401), and an immunoglobulin Y egg yolk antibody (e.g. AGY). In a specific embodiment, the agent is a cytokine. Exemplary cytokines include, but are not limited to, interferon-α. In a specific embodiment, the molecule (e.g., therapeutic molecule) comprises is a hormone. Exemplary hormones include, but are not limited to, desmopressin (e.g. DDAVP). In a specific embodiment, the molecule (e.g., therapeutic molecule) comprises a small molecule. Exemplary small molecules include, but are not limited to, cyclosporin A (e.g. Neoral). The molecule may be administered to the bloodstream of the subject. The molecule may be administered intravenously or subcutaneously. The molecule may be administered by oral delivery, buccal delivery, nasal delivery or inhalation delivery, including for delivery to systemic circulation or Lamina propria.

In another aspect is provided a method of delivering a molecule (e.g., therapeutic molecule) to a mucosal lumen of a subject, including, for example, as measured by any assays or models of delivery as described herein. The method comprises administering to the subject any VHH domain and an agent (e.g., therapeutic molecule) described herein, or an effective amount of any VHH domain and an agent (e.g., therapeutic molecule) described herein. In a related aspect is provided a method for transporting small molecule and protein-based entities across the mucosal epithelial cell by exploiting pIgR-mediated transcytosis, including, for example, as measure by any assays or models of transport as described herein. In some embodiments, the cell is a mucosal epithelial cell. In some embodiments, the cell is a cancer cell. Exemplary cancer cells include, but are not limited to, a lung cancer cell, an esophageal cancer cell, a stomach cancer cell, a duodenal cancer cell, a liver cancer cell, a bladder cancer cell, a sinus cancer cell, a nasal cavity cancer cell, an endometrial cancer cell or a colorectal cancer cell. The cell may be in a subject. The molecule (e.g., therapeutic molecule) may comprise an antibody or fragment thereof, a peptide, a vaccine, a small molecule, a polynucleotide, a molecule comprising a radioactive isotope, a toxin, an enzyme, an anticoagulant, a hormone, a cytokine, an anti-inflammatory molecule, an RNAi, an mRNA, a self-replicating RNA, an antibiotic, and an antibody-antibiotic conjugate as described herein. In certain embodiments, the molecule (e.g., therapeutic molecule) comprises an antibiotic (e.g., a macrolide antibiotic, a fluoroquinolone, a tetracycline, amoxicillin, ceftriaxone, penicillin G, linezolid, moxifloxacin, or azithromycin). In a specific embodiment, the molecule (e.g., therapeutic molecule) comprises a peptide. Exemplary peptides include, but are not limited to, an octreotide (e.g. Mycapssa), insulin or a derivative thereof (e.g. Capsulin OAD, ORMD-0801, Tregopil, HDV Insulin, Oshadi Icp, Dance 501, Exubera, Afrezza, Oral-lyn, MSL001-PH-2-1, NanoCelle Insulin), an insulin-mimic peptide, a semaglutide (e.g. NN9924), a leuprolide (e.g. Ovarest), a glucagon-like peptide 1 (e.g. TTP273), a glucagon-like-peptide-1-mimic peptides, an IL-23 receptor antagonist peptide (e.g., PTG-200), a salmon calcitonin (e.g. TBRIA), a desmopressin (e.g. DDAVP), a calcitonin (e.g. Miacalcin), an oxytocin (e.g. Syntocinon), and a nafarelin (e.g. Synarel). In a specific embodiment, the molecule (e.g., therapeutic molecule) comprises a vaccine. Exemplary vaccines are useful for preventing inventions, including infections from Vibrio, Cholera, Typhoid, Rotavirus, Tuberculosis, HIV, Flu, Ebola, and Sendai. Exemplary vaccines include, but are not limited to, connaught strain BCG (e.g. BCG vaccine), a live attenuated cholera vaccine (e.g. Vaxchora), a live attenuated Salmonella enterica subsp. enterica seravar typi Ty21a (e.g. Vivotif), a live, noncovalent, human attenuated rotavirus strain (e.g. Rotarix), a live pentavalent bovine attenuated rotavirus strain (e.g. RotaTeq), a recombinant modified vaccinia virus Ankara expressing antigen 85A (MVA85A) (e.g. MVA85A), a live attenuated Bordetella pertussis (e.g. BPZE1), a flu vaccine (e.g. PUR003, INFLUSOME-VAC, FluMist Quadrivalent), a Tuberculosis vaccine (e.g. Ad5Ag85 Å, Tuberculosis vaccine), an HIV vaccine (e.g. EuroNeut41, HIV vaccine), an inactivated H5N1 influenza vaccine (e.g. GelVac), an RSVcps2 vaccine (e.g. Respiratory syncytial virus vaccine), a Shigellosis vaccine (e.g. Invaplex 50), an ebola vaccine (e.g. Ebola vaccine), and a Sendai vaccine (e.g. Sendai vaccine). In a specific embodiment, the molecule (e.g., therapeutic molecule) comprises an antibody or fragment thereof. Exemplary antibodies or fragments thereof include, but are not limited to, an antitumour necrosis factor antibody (e.g. AVX-470), an anti-TNF-alpha antibody (e.g., infliximab), an anti-IL23 antibody (e.g., guselkumab), an antibody that binds to a receptor of IL23, an anti-IL12 and anti-IL23 antibody (e.g., uspekinumab), muromonab (e.g. OKT3), a homeopathic antibody (e.g. TAO1), an anti-CD3 antibody (e.g. aCD3, TZLS-401), and an immunoglobulin Y egg yolk antibody (e.g. AGY). In a specific embodiment, the agent is a cytokine. Exemplary cytokines include, but are not limited to, interferon-α. In a specific embodiment, the molecule (e.g., therapeutic molecule) comprises is a hormone. Exemplary hormones include, but are not limited to, desmopressin (e.g. DDAVP). In a specific embodiment, the molecule (e.g., therapeutic molecule) comprises a small molecule. Exemplary small molecules include, but are not limited to, cyclosporin A (e.g. Neoral). The molecule may be administered to the bloodstream of the subject. The molecule may be administered intravenously or subcutaneously. The molecule may be administered by oral delivery, buccal delivery, nasal delivery or inhalation delivery, including for delivery to systemic circulation or Lamina propria.

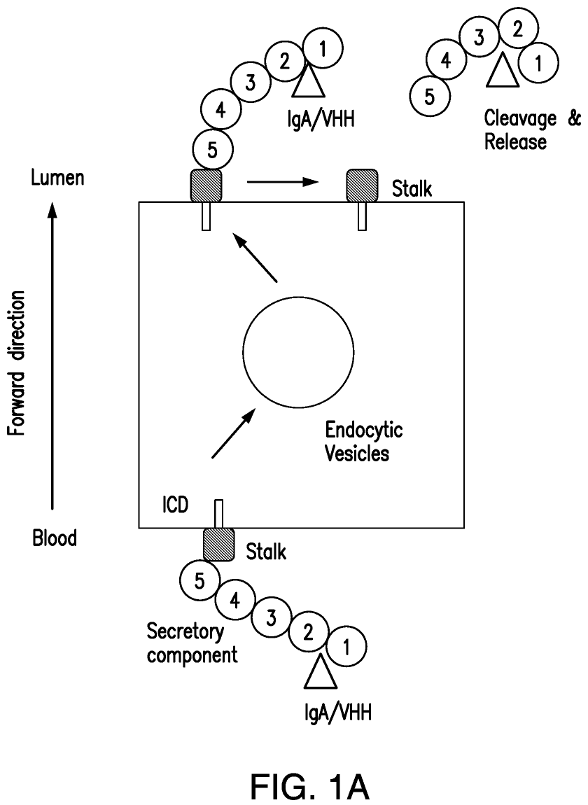

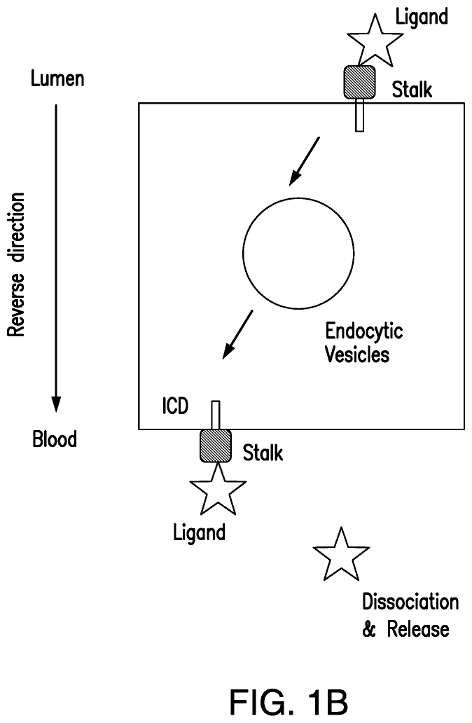

The schematic shown in FIG. 1B illustrates how molecules binding to the stalk region of the pIgR ectodomain (any artificial ligand) can transcytose the epithelial cell from the apical to the basolateral direction and reach the blood from mucosal lumen.

In another aspect is provided a method of delivering a molecule to a mucosal lumen of a subject, including, for example, as measured by any assays or models of delivery as described herein. The method comprises administering to the subject any VHH domain and an agent (e.g., therapeutic molecule) described herein, or an effective amount of any VHH domain and an agent (e.g., therapeutic molecule) described herein. In certain embodiments, the mucosal lumen is in the lung or in the gastrointestinal tract of the subject. The molecule (e.g. therapeutic molecule) may comprise an antibody or fragment thereof, a peptide, a vaccine, a small molecule, a polynucleotide, a molecule comprising a radioactive isotope, a toxin, an enzyme, an anticoagulant, a hormone, a cytokine, an anti-inflammatory molecule, an RNAi, an mRNA, a self-replicating RNA, an antibiotic, and an antibody-antibiotic conjugate as described herein. In certain embodiments, the molecule (e.g., therapeutic molecule) comprises an antibiotic (e.g., a macrolide antibiotic, a fluoroquinolone, a tetracycline, amoxicillin, ceftriaxone, penicillin G, linezolid, moxifloxacin, or azithromycin.) The molecule (e.g., therapeutic molecule) may comprise an antibody or fragment thereof, a peptide, a vaccine, a small molecule, a polynucleotide, a molecule comprising a radioactive isotope, a toxin, an enzyme, an anticoagulant, a hormone, a cytokine, an anti-inflammatory molecule, an RNAi, an mRNA, a self-replicating RNA, an antibiotic, and an antibody-antibiotic conjugate as described herein. In certain embodiments, the molecule (e.g., therapeutic molecule) comprises an antibiotic (e.g., a macrolide antibiotic, a fluoroquinolone, a tetracycline, amoxicillin, ceftriaxone, penicillin G, linezolid, moxifloxacin, or azithromycin). In a specific embodiment, the molecule (e.g., therapeutic molecule) comprises a peptide. Exemplary peptides include, but are not limited to, an octreotide (e.g. Mycapssa), insulin or a derivative thereof (e.g. Capsulin OAD, ORMD-0801, Tregopil, HDV Insulin, Oshadi Icp, Dance 501, Exubera, Afrezza, Oral-lyn, MSL001-PH-2-1, NanoCelle Insulin), an insulin-mimic peptide, a semaglutide (e.g. NN9924), a leuprolide (e.g. Ovarest), a glucagon-like peptide 1 (e.g. TTP273), a glucagon-like-peptide-1-mimic peptides, an IL-23 receptor antagonist peptide (e.g., PTG-200), a salmon calcitonin (e.g. TBRIA), a desmopressin (e.g. DDAVP), a calcitonin (e.g. Miacalcin), an oxytocin (e.g. Syntocinon), and a nafarelin (e.g. Synarel). In a specific embodiment, the molecule (e.g., therapeutic molecule) comprises a vaccine. Exemplary vaccines are useful for preventing inventions, including infections from Vibrio, Cholera, Typhoid, Rotavirus, Tuberculosis, HIV, Flu, Ebola, and Sendai. Exemplary vaccines include, but are not limited to, connaught strain BCG (e.g. BCG vaccine), a live attenuated cholera vaccine (e.g. Vaxchora), a live attenuated Salmonella enterica subsp. enterica seravar typi Ty21a (e.g. Vivotif), a live, noncovalent, human attenuated rotavirus strain (e.g. Rotarix), a live pentavalent bovine attenuated rotavirus strain (e.g. RotaTeq), a recombinant modified vaccinia virus Ankara expressing antigen 85A (MVA85A) (e.g. MVA85A), a live attenuated Bordetella pertussis (e.g. BPZE1), a flu vaccine (e.g. PUR003, INFLUSOME-VAC, FluMist Quadrivalent), a Tuberculosis vaccine (e.g. Ad5Ag85 Å, Tuberculosis vaccine), an HIV vaccine (e.g. EuroNeut41, HIV vaccine), an inactivated H5N1 influenza vaccine (e.g. GelVac), an RSVcps2 vaccine (e.g. Respiratory syncytial virus vaccine), a Shigellosis vaccine (e.g. Invaplex 50), an ebola vaccine (e.g. Ebola vaccine), and a Sendai vaccine (e.g. Sendai vaccine). In a specific embodiment, the molecule (e.g., therapeutic molecule) comprises an antibody or fragment thereof. Exemplary antibodies or fragments thereof include, but are not limited to, an antitumour necrosis factor antibody (e.g. AVX-470), an anti-TNF-alpha antibody (e.g., infliximab), an anti-IL23 antibody (e.g., guselkumab), an antibody that binds to a receptor of IL23, an anti-IL12 and anti-IL23 antibody (e.g., uspekinumab), muromonab (e.g. OKT3), a homeopathic antibody (e.g. TAO1), an anti-CD3 antibody (e.g. aCD3, TZLS-401), and an immunoglobulin Y egg yolk antibody (e.g. AGY). In a specific embodiment, the agent is a cytokine. Exemplary cytokines include, but are not limited to, interferon-α. In a specific embodiment, the molecule (e.g., therapeutic molecule) comprises is a hormone. Exemplary hormones include, but are not limited to, desmopressin (e.g. DDAVP). In a specific embodiment, the molecule (e.g., therapeutic molecule) comprises a small molecule. Exemplary small molecules include, but are not limited to, cyclosporin A (e.g. Neoral). The molecule may be administered to the bloodstream of the subject. The molecule may be administered intravenously or subcutaneously. The molecule may be administered by oral delivery, buccal delivery, nasal delivery or inhalation delivery, including for delivery to systemic circulation or Lamina propria.

In another aspect is provided a method of delivering a molecule to an organ of a subject, including, for example, as measured by any assays or models of delivery as described herein. The method comprises administering to the subject any VHH domain and an agent (e.g., therapeutic molecule) described herein, or an effective amount of any VHH domain and an agent (e.g., therapeutic molecule) described herein. The organ may be the small intestine, large intestine, stomach, esophagus, salivary gland, lung, vagina, uterus, or lacrimal gland. In specific embodiments, the organ is a lung. The molecule (e.g., therapeutic molecule) may comprise an antibody or fragment thereof, a peptide, a vaccine, a small molecule, a polynucleotide, a molecule comprising a radioactive isotope, a toxin, an enzyme, an anticoagulant, a hormone, a cytokine, an anti-inflammatory molecule, an RNAi, an mRNA, a self-replicating RNA, an antibiotic, and an antibody-antibiotic conjugate as described herein. In certain embodiments, the molecule (e.g., therapeutic molecule) comprises an antibiotic (e.g., a macrolide antibiotic, a fluoroquinolone, a tetracycline, amoxicillin, ceftriaxone, penicillin G, linezolid, moxifloxacin, or azithromycin.) The molecule may be administered to the bloodstream of the subject. In a specific embodiment, the molecule (e.g., therapeutic molecule) comprises a peptide. Exemplary peptides include, but are not limited to, an octreotide (e.g. Mycapssa), insulin or a derivative thereof (e.g. Capsulin OAD, ORMD-0801, Tregopil, HDV Insulin, Oshadi Icp, Dance 501, Exubera, Afrezza, Oral-lyn, MSL001-PH-2-1, NanoCelle Insulin), an insulin-mimic peptide, a semaglutide (e.g. NN9924), a leuprolide (e.g. Ovarest), a glucagon-like peptide 1 (e.g. TTP273), a glucagon-like-peptide-1-mimic peptides, an IL-23 receptor antagonist peptide (e.g., PTG-200), a salmon calcitonin (e.g. TBRIA), a desmopressin (e.g. DDAVP), a calcitonin (e.g. Miacalcin), an oxytocin (e.g. Syntocinon), and a nafarelin (e.g. Synarel). In a specific embodiment, the molecule (e.g., therapeutic molecule) comprises a vaccine. Exemplary vaccines are useful for preventing inventions, including infections from Vibrio, Cholera, Typhoid, Rotavirus, Tuberculosis, HIV, Flu, Ebola, and Sendai. Exemplary vaccines include, but are not limited to, connaught strain BCG (e.g. BCG vaccine), a live attenuated cholera vaccine (e.g. Vaxchora), a live attenuated Salmonella enterica subsp. enterica seravar typi Ty21a (e.g. Vivotif), a live, noncovalent, human attenuated rotavirus strain (e.g. Rotarix), a live pentavalent bovine attenuated rotavirus strain (e.g. RotaTeq), a recombinant modified vaccinia virus Ankara expressing antigen 85A (MVA85A) (e.g. MVA85A), a live attenuated Bordetella pertussis (e.g. BPZE1), a flu vaccine (e.g. PUR003, INFLUSOME-VAC, FluMist Quadrivalent), a Tuberculosis vaccine (e.g. Ad5Ag85 Å, Tuberculosis vaccine), an HIV vaccine (e.g. EuroNeut41, HIV vaccine), an inactivated H5N1 influenza vaccine (e.g. GelVac), an RSVcps2 vaccine (e.g. Respiratory syncytial virus vaccine), a Shigellosis vaccine (e.g. Invaplex 50), an ebola vaccine (e.g. Ebola vaccine), and a Sendai vaccine (e.g. Sendai vaccine). In a specific embodiment, the molecule (e.g., therapeutic molecule) comprises an antibody or fragment thereof. Exemplary antibodies or fragments thereof include, but are not limited to, an antitumour necrosis factor antibody (e.g. AVX-470), an anti-TNF-alpha antibody (e.g., infliximab), an anti-IL23 antibody (e.g., guselkumab), an antibody that binds to a receptor of IL23, an anti-IL12 and anti-IL23 antibody (e.g., uspekinumab), muromonab (e.g. OKT3), a homeopathic antibody (e.g. TAO1), an anti-CD3 antibody (e.g. aCD3, TZLS-401), and an immunoglobulin Y egg yolk antibody (e.g. AGY). In a specific embodiment, the agent is a cytokine. Exemplary cytokines include, but are not limited to, interferon-α. In a specific embodiment, the molecule (e.g., therapeutic molecule) comprises is a hormone. Exemplary hormones include, but are not limited to, desmopressin (e.g. DDAVP). In a specific embodiment, the molecule (e.g., therapeutic molecule) comprises a small molecule. Exemplary small molecules include, but are not limited to, cyclosporin A (e.g. Neoral). The molecule may be administered intravenously or subcutaneously. The molecule may be administered by oral delivery, buccal delivery, nasal delivery or inhalation delivery, including for delivery to systemic circulation or Lamina propria.

In another aspect is provided a method of delivering a molecule to systemic circulation in a subject, including, for example, as measured by any assays or models of delivery as described herein. The method comprises administering to the subject any VHH domain and an agent (e.g., therapeutic molecule) described herein, or an effective amount of any molecule comprising a VHH domain and an agent (e.g., therapeutic molecule) described herein. The molecule (e.g. therapeutic molecule) may comprise an antibody or fragment thereof, a peptide, a vaccine, a small molecule, a polynucleotide, a molecule comprising a radioactive isotope, a toxin, an enzyme, an anticoagulant, a hormone, a cytokine, an anti-inflammatory molecule, an RNAi, an mRNA, a self-replicating RNA, an antibiotic, and an antibody-antibiotic conjugate as described herein. In certain embodiments, the molecule (e.g., therapeutic molecule) comprises an antibiotic (e.g., a macrolide antibiotic, a fluoroquinolone, a tetracycline, amoxicillin, ceftriaxone, penicillin G, linezolid, moxifloxacin, or azithromycin.) The molecule (e.g., therapeutic molecule) may comprise an antibody or fragment thereof, a peptide, a vaccine, a small molecule, a polynucleotide, a molecule comprising a radioactive isotope, a toxin, an enzyme, an anticoagulant, a hormone, a cytokine, an anti-inflammatory molecule, an RNAi, an mRNA, a self-replicating RNA, an antibiotic, and an antibody-antibiotic conjugate as described herein. In certain embodiments, the molecule (e.g., therapeutic molecule) comprises an antibiotic (e.g., a macrolide antibiotic, a fluoroquinolone, a tetracycline, amoxicillin, ceftriaxone, penicillin G, linezolid, moxifloxacin, or azithromycin). In a specific embodiment, the molecule (e.g., therapeutic molecule) comprises a peptide. Exemplary peptides include, but are not limited to, an octreotide (e.g. Mycapssa), insulin or a derivative thereof (e.g. Capsulin OAD, ORMD-0801, Tregopil, HDV Insulin, Oshadi Icp, Dance 501, Exubera, Afrezza, Oral-lyn, MSL001-PH-2-1, NanoCelle Insulin), an insulin-mimic peptide, a semaglutide (e.g. NN9924), a leuprolide (e.g. Ovarest), a glucagon-like peptide 1 (e.g. TTP273), a glucagon-like-peptide-1-mimic peptides, an IL-23 receptor antagonist peptide (e.g., PTG-200), a salmon calcitonin (e.g. TBRIA), a desmopressin (e.g. DDAVP), a calcitonin (e.g. Miacalcin), an oxytocin (e.g. Syntocinon), and a nafarelin (e.g. Synarel). In a specific embodiment, the molecule (e.g., therapeutic molecule) comprises a vaccine. Exemplary vaccines are useful for preventing inventions, including infections from Vibrio, Cholera, Typhoid, Rotavirus, Tuberculosis, HIV, Flu, Ebola, and Sendai. Exemplary vaccines include, but are not limited to, connaught strain BCG (e.g. BCG vaccine), a live attenuated cholera vaccine (e.g. Vaxchora), a live attenuated Salmonella enterica subsp. enterica seravar typi Ty21a (e.g. Vivotif), a live, noncovalent, human attenuated rotavirus strain (e.g. Rotarix), a live pentavalent bovine attenuated rotavirus strain (e.g. RotaTeq), a recombinant modified vaccinia virus Ankara expressing antigen 85A (MVA85A) (e.g. MVA85A), a live attenuated Bordetella pertussis (e.g. BPZE1), a flu vaccine (e.g. PUR003, INFLUSOME-VAC, FluMist Quadrivalent), a Tuberculosis vaccine (e.g. Ad5Ag85 Å, Tuberculosis vaccine), an HIV vaccine (e.g. EuroNeut41, HIV vaccine), an inactivated H5N1 influenza vaccine (e.g. GelVac), an RSVcps2 vaccine (e.g. Respiratory syncytial virus vaccine), a Shigellosis vaccine (e.g. Invaplex 50), an ebola vaccine (e.g. Ebola vaccine), and a Sendai vaccine (e.g. Sendai vaccine). In a specific embodiment, the molecule (e.g., therapeutic molecule) comprises an antibody or fragment thereof. Exemplary antibodies or fragments thereof include, but are not limited to, an antitumour necrosis factor antibody (e.g. AVX-470), an anti-TNF-alpha antibody (e.g., infliximab), an anti-IL23 antibody (e.g., guselkumab), an antibody that binds to a receptor of IL23, an anti-IL12 and anti-IL23 antibody (e.g., uspekinumab), muromonab (e.g. OKT3), a homeopathic antibody (e.g. TAO1), an anti-CD3 antibody (e.g. aCD3, TZLS-401), and an immunoglobulin Y egg yolk antibody (e.g. AGY). In a specific embodiment, the agent is a cytokine. Exemplary cytokines include, but are not limited to, interferon-α. In a specific embodiment, the molecule (e.g., therapeutic molecule) comprises is a hormone. Exemplary hormones include, but are not limited to, desmopressin (e.g. DDAVP). In a specific embodiment, the molecule (e.g., therapeutic molecule) comprises a small molecule. Exemplary small molecules include, but are not limited to, cyclosporin A (e.g. Neoral). The molecule may be administered to the bloodstream of the subject. The molecule may be administered intravenously or subcutaneously. The molecule may be administered by oral delivery, buccal delivery, nasal delivery or inhalation delivery, including for delivery to systemic circulation or Lamina propria.

In another aspect is provided a method of delivering a molecule to Lamina propria of a subject, including, for example, as measured by any assays or models of delivery as described herein. The method comprises administering to the subject any VHH domain and an agent (e.g., therapeutic molecule) described herein, or an effective amount of any a VHH domain and an agent (e.g., therapeutic molecule) described herein. The molecule (e.g. therapeutic molecule) may comprise an antibody or fragment thereof, a peptide, a vaccine, a small molecule, a polynucleotide, a molecule comprising a radioactive isotope, a toxin, an enzyme, an anticoagulant, a hormone, a cytokine, an anti-inflammatory molecule, an RNAi, an mRNA, a self-replicating RNA, an antibiotic, and an antibody-antibiotic conjugate as described herein. In certain embodiments, the molecule (e.g., therapeutic molecule) comprises an antibiotic (e.g., a macrolide antibiotic, a fluoroquinolone, a tetracycline, amoxicillin, ceftriaxone, penicillin G, linezolid, moxifloxacin, or azithromycin.) The molecule (e.g., therapeutic molecule) may comprise an antibody or fragment thereof, a peptide, a vaccine, a small molecule, a polynucleotide, a molecule comprising a radioactive isotope, a toxin, an enzyme, an anticoagulant, a hormone, a cytokine, an anti-inflammatory molecule, an RNAi, an mRNA, a self-replicating RNA, an antibiotic, and an antibody-antibiotic conjugate as described herein. In certain embodiments, the molecule (e.g., therapeutic molecule) comprises an antibiotic (e.g., a macrolide antibiotic, a fluoroquinolone, a tetracycline, amoxicillin, ceftriaxone, penicillin G, linezolid, moxifloxacin, or azithromycin). In a specific embodiment, the molecule (e.g., therapeutic molecule) comprises a peptide. Exemplary peptides include, but are not limited to, an octreotide (e.g. Mycapssa), insulin or a derivative thereof (e.g. Capsulin OAD, ORMD-0801, Tregopil, HDV Insulin, Oshadi Icp, Dance 501, Exubera, Afrezza, Oral-lyn, MSL001-PH-2-1, NanoCelle Insulin), an insulin-mimic peptide, a semaglutide (e.g. NN9924), a leuprolide (e.g. Ovarest), a glucagon-like peptide 1 (e.g. TTP273), a glucagon-like-peptide-1-mimic peptides, an IL-23 receptor antagonist peptide (e.g., PTG-200), a salmon calcitonin (e.g. TBRIA), a desmopressin (e.g. DDAVP), a calcitonin (e.g. Miacalcin), an oxytocin (e.g. Syntocinon), and a nafarelin (e.g. Synarel). In a specific embodiment, the molecule (e.g., therapeutic molecule) comprises a vaccine. Exemplary vaccines are useful for preventing inventions, including infections from Vibrio, Cholera, Typhoid, Rotavirus, Tuberculosis, HIV, Flu, Ebola, and Sendai. Exemplary vaccines include, but are not limited to, connaught strain BCG (e.g. BCG vaccine), a live attenuated cholera vaccine (e.g. Vaxchora), a live attenuated Salmonella enterica subsp. enterica seravar typi Ty21a (e.g. Vivotif), a live, noncovalent, human attenuated rotavirus strain (e.g. Rotarix), a live pentavalent bovine attenuated rotavirus strain (e.g. RotaTeq), a recombinant modified vaccinia virus Ankara expressing antigen 85A (MVA85A) (e.g. MVA85A), a live attenuated Bordetella pertussis (e.g. BPZE1), a flu vaccine (e.g. PUR003, INFLUSOME-VAC, FluMist Quadrivalent), a Tuberculosis vaccine (e.g. Ad5Ag85 Å, Tuberculosis vaccine), an HIV vaccine (e.g. EuroNeut41, HIV vaccine), an inactivated H5N1 influenza vaccine (e.g. GelVac), an RSVcps2 vaccine (e.g. Respiratory syncytial virus vaccine), a Shigellosis vaccine (e.g. Invaplex 50), an ebola vaccine (e.g. Ebola vaccine), and a Sendai vaccine (e.g. Sendai vaccine). In a specific embodiment, the molecule (e.g., therapeutic molecule) comprises an antibody or fragment thereof. Exemplary antibodies or fragments thereof include, but are not limited to, an antitumour necrosis factor antibody (e.g. AVX-470), an anti-TNF-alpha antibody (e.g., infliximab), an anti-IL23 antibody (e.g., guselkumab), an antibody that binds to a receptor of IL23, an anti-IL12 and anti-IL23 antibody (e.g., uspekinumab), muromonab (e.g. OKT3), a homeopathic antibody (e.g. TAO1), an anti-CD3 antibody (e.g. aCD3, TZLS-401), and an immunoglobulin Y egg yolk antibody (e.g. AGY). In a specific embodiment, the agent is a cytokine. Exemplary cytokines include, but are not limited to, interferon-α. In a specific embodiment, the molecule (e.g., therapeutic molecule) comprises is a hormone. Exemplary hormones include, but are not limited to, desmopressin (e.g. DDAVP). In a specific embodiment, the molecule (e.g., therapeutic molecule) comprises a small molecule. Exemplary small molecules include, but are not limited to, cyclosporin A (e.g. Neoral). The molecule may be administered to the bloodstream of the subject. The molecule may be administered intravenously or subcutaneously. The molecule may be administered by oral delivery, buccal delivery, nasal delivery or inhalation delivery, including for delivery to systemic circulation or Lamina propria.

The VHH domains and molecules comprising VHH domains (e.g., therapeutic molecules, including conjugates, such as bioconjugates) described herein may be used to deliver cytokines and anti-inflammatory antibodies into lung mucosa for immunology indications (asthma), delivery of anti-inflammatory antibodies into intestinal mucosa for Intestinal bowel disease and Ulcerative colitis, delivery of antibody-antibiotic conjugates for clearing mucosal infections, pIgR-mediated increase in the biodistribution of endometrial and colorectal cancer targeting biologics in mucosa, and radiolabeled VHH-Fc molecules for mucosal PET-CT imaging.

The VHH domains and molecules comprising VHH domains (e.g., therapeutic molecules, including conjugates, such as bioconjugates) described herein may be used to improve the stability and PK for oral delivery of anti-inflammatory antibodies for Intestinal bowel disease and Ulcerative colitis. The VHH domain may be co-administered with the anti-inflammatory antibody. The VHH domain may also be conjugated, chemically or genetically, to the anti-inflammatory antibody. VHH domains or molecules comprising a VHH domain and an agent (e.g., therapeutic molecules) described herein be used for testing unexplored diagnostic and therapeutic applications in the pIgR space, such as delivery of cytokines and anti-inflammatory antibodies into lung for immunology indications, delivery of antibody-antibiotic conjugates for clearing mucosal infections, pIgR-mediated increase in the biodistribution of endometrial and colorectal cancer targeting biologics in mucosa, and radiolabeled VHH-Fc molecules for mucosal imaging.

The disclosure also provides related nucleic acids, recombinant expression vectors, host cells, populations of cells, antibodies, or antigen binding portions thereof, and pharmaceutical compositions relating to the VHH domains or molecules comprising a VHH domain and an agent (e.g., therapeutic molecules) described herein.

Several aspects of the invention are described below, with reference to examples for illustrative purposes only. It should be understood that numerous specific details, relationships, and methods are set forth to provide a full understanding of the invention. One having ordinary skill in the relevant art, however, will readily recognize that the invention can be practiced without one or more of the specific details or practiced with other methods, protocols, reagents, cell lines and animals. The present invention is not limited by the illustrated ordering of acts or events, as some acts may occur in different orders and/or concurrently with other acts or events. Furthermore, not all illustrated acts, steps or events are required to implement a methodology in accordance with the present invention. Many of the techniques and procedures described, or referenced herein, are well understood and commonly employed using conventional methodology by those skilled in the art.

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. It will be further understood that terms, such as those defined in commonly-used dictionaries, should be interpreted as having a meaning that is consistent with their meaning in the context of the relevant art and/or as otherwise defined herein.

### 5.1 Definitions

The terms “antibody” and “antibodies” refer to monoclonal antibodies, multispecific antibodies, human antibodies, humanized antibodies, chimeric antibodies, single-chain Fvs (scFv), single chain antibodies, Fab fragments, F(ab′) fragments, disulfide-linked Fvs (sdFv), intrabodies, minibodies, and diabodies, and epitope-binding fragments of any of the above. The terms “antibody” and “antibodies” also refer to covalent diabodies such as those disclosed in U.S. Pat. Appl. Pub. 2007/0004909 and Ig-DARTS such as those disclosed in U.S. Pat. Appl. Pub. 2009/0060910. Antibodies include immunoglobulin molecules and immunologically active fragments of immunoglobulin molecules, i.e., molecules that contain an antigen-binding site. Immunoglobulin molecules can be of any type (e.g., IgG, IgE, IgM, IgD, IgA and IgY), class (e.g., IgG1, IgG2, IgG3, IgG4, IgM1, IgM2, IgA1 and IgA2) or subclass.

The terms “express” and “expression” mean allowing or causing the information in a gene or DNA sequence to become produced, for example producing a protein by activating the cellular functions involved in transcription and translation of a corresponding gene or DNA sequence. A DNA sequence is expressed in or by a cell to form an “expression product” such as a protein. The expression product itself, e.g., the resulting protein, may also be said to be “expressed” by the cell. An expression product can be characterized as intracellular, extracellular or transmembrane.

The term “transfection” means the introduction of a “foreign” (i.e., extrinsic or extracellular) nucleic acid into a cell using recombinant DNA technology. The term “genetic modification” means the introduction of a “foreign” (i.e., extrinsic or extracellular) gene, DNA or RNA sequence to a host cell, so that the host cell will express the introduced gene or sequence to produce a desired substance, typically a protein or enzyme coded by the introduced gene or sequence. The introduced gene or sequence may also be called a “cloned” or “foreign” gene or sequence, may include regulatory or control sequences operably linked to polynucleotide encoding the chimeric antigen receptor, such as start, stop, promoter, signal, secretion, or other sequences used by a cell's genetic machinery. The gene or sequence may include nonfunctional sequences or sequences with no known function. A host cell that receives and expresses introduced DNA or RNA has been “genetically engineered.” The DNA or RNA introduced to a host cell can come from any source, including cells of the same genus or species as the host cell, or from a different genus or species.

The term “transduction” means the introduction of a foreign nucleic acid into a cell using a viral vector.

The term “regulatory element” refers to any cis-acting genetic element that controls some aspect of the expression of nucleic acid sequences. In some embodiments, the term “promoter” comprises essentially the minimal sequences required to initiate transcription. In some embodiments, the term “promoter” includes the sequences to start transcription, and in addition, also include sequences that can upregulate or downregulate transcription, commonly termed “enhancer elements” and “repressor elements”, respectively.

As used herein, the term “operatively linked,” and similar phrases, when used in reference to nucleic acids or amino acids, refer to the operational linkage of nucleic acid sequences or amino acid sequence, respectively, placed in functional relationships with each other. For example, an operatively linked promoter, enhancer elements, open reading frame, 5′ and 3′ UTR, and terminator sequences result in the accurate production of a nucleic acid molecule (e.g., RNA). In some embodiments, operatively linked nucleic acid elements result in the transcription of an open reading frame and ultimately the production of a polypeptide (i.e., expression of the open reading frame). As another example, an operatively linked peptide is one in which the functional domains are placed with appropriate distance from each other to impart the intended function of each domain.

The term “effective” applied to dose or amount refers to that quantity of a compound or pharmaceutical composition that is sufficient to result in a desired activity upon administration to a subject in need thereof. Note that when a combination of active ingredients is administered, the effective amount of the combination may or may not include amounts of each ingredient that would have been effective if administered individually. The exact amount required will vary from subject to subject, depending on the species, age, and general condition of the subject, the severity of the condition being treated, the particular drug or drugs employed, the mode of administration, and the like.

The terms “treat” or “treatment” refer to therapeutic treatment wherein the object is to slow down (lessen) an undesired physiological change or disease, or provide a beneficial or desired clinical outcome during treatment. Beneficial or desired clinical outcomes include alleviation of symptoms, diminishment of extent of disease, stabilized (i.e., not worsening) state of disease, delay or slowing of disease progression, amelioration or palliation of the disease state, and/or remission (whether partial or total), whether detectable or undetectable. “Treatment” can also mean prolonging survival as compared to expected survival if a subject was not receiving treatment. Those in need of treatment include those subjects already with the undesired physiological change or disease as well as those subjects prone to have the physiological change or disease.

A “therapeutically effective amount” or “effective amount”, used interchangeably herein, refers to an amount effective, at dosages and for periods of time necessary, to achieve a desired therapeutic result. A therapeutically effective amount may vary according to factors such as the disease state, age, sex, and weight of the individual, and the ability of a therapeutic or a combination of therapeutics to elicit a desired response in the individual. Example indicators of an effective therapeutic or combination of therapeutics that include, for example, improved well-being of the patient, reduction of a tumor burden, arrested or slowed growth of a tumor, and/or absence of metastasis of cancer cells to other locations in the body. Other exemplary indicators of an effective therapeutic or combination of therapeutics include reduction in disease activity index, such as Crohn's Disease Activity Index (CDAI) or achieving glycemic control.

As used herein, the term “subject” refers to an animal. The terms “subject” and “patient” may be used interchangeably herein in reference to a subject. As such, a “subject” includes a human that is being treated for a disease, or prevention of a disease, as a patient. The methods described herein may be used to treat an animal subject belonging to any classification. Examples of such animals include mammals. Mammals, include, but are not limited to, mammals of the order Rodentia, such as mice and hamsters, and mammals of the order Logomorpha, such as rabbits. The mammals may be from the order Carnivora, including Felines (cats) and Canines (dogs). The mammals may be from the order Artiodactyla, including Bovines (cows) and Swines (pigs) or of the order Perssodactyla, including Equines (horses). The mammals may be of the order Primates, Ceboids, or Simoids (monkeys) or of the order Anthropoids (humans and apes). In one embodiment, the mammal is a human.

The phrase “pharmaceutically acceptable”, as used in connection with compositions described herein, refers to molecular entities and other ingredients of such compositions that are physiologically tolerable and do not typically produce untoward reactions when administered to a mammal (e.g., a human). Preferably, the term “pharmaceutically acceptable” means approved by a regulatory agency of the Federal or a state government or listed in the U.S. Pharmacopeia or other generally recognized pharmacopeia for use in mammals, and more particularly in humans.

The term “protein” is used herein encompasses all kinds of naturally occurring and synthetic proteins, including protein fragments of all lengths, fusion proteins and modified proteins, including without limitation, glycoproteins, as well as all other types of modified proteins (e.g., proteins resulting from phosphorylation, acetylation, myristoylation, palmitoylation, glycosylation, oxidation, formylation, amidation, polyglutamylation, ADP-ribosylation, pegylation, biotinylation, etc.).

The terms “nucleic acid”, “nucleotide”, and “polynucleotide” encompass both DNA and RNA unless specified otherwise. By a “nucleic acid sequence” or “nucleotide sequence” is meant the nucleic acid sequence encoding an amino acid; these terms may also refer to the nucleic acid sequence including the portion coding for any amino acids added as an artifact of cloning, including any amino acids coded for by linkers.

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or a RNA may also include introns to the extent that the nucleotide sequence encoding the protein may in some version contain an intron(s).

The term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide.

The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Alternatively, the carrier can be a solid dosage form carrier, including but not limited to one or more of a binder (for compressed pills), a glidant, an encapsulating agent, a flavorant, and a colorant. Suitable pharmaceutical carriers are described in “Remington's Pharmaceutical Sciences” by E. W. Martin.

The term “about” or “approximately” includes being within a statistically meaningful range of a value. Such a range can be within an order of magnitude, preferably within 50%, more preferably within 20%, still more preferably within 10%, and even more preferably within 5% of a given value or range. The allowable variation encompassed by the term “about” or “approximately” depends on the particular system under study, and can be readily appreciated by one of ordinary skill in the art.

The terminology used herein is for the purpose of describing particular embodiments only and is not intended to be limiting. As used herein, the indefinite articles “a”, “an” and “the” should be understood to include plural reference unless the context clearly indicates otherwise.

“Single domain antibody” or “sdAb” as used herein refers to a single monomeric variable antibody domain and which is capable of antigen binding (e.g., single domain antibodies that bind to pIgR). Single domain antibodies include VHH domains as described herein. Examples of single domain antibodies include, but are not limited to, antibodies naturally devoid of light chains such as those from Camelidae species (e.g., llama), single domain antibodies derived from conventional 4-chain antibodies, engineered antibodies and single domain scaffolds other than those derived from antibodies. Single domain antibodies may be derived from any species including, but not limited to mouse, human, camel, llama, goat, rabbit, and bovine. For example, a single domain antibody can be derived from antibodies raised in Camelidae species, for example in camel, llama, dromedary, alpaca and guanaco, as described herein. Other species besides Camelidae may produce heavy chain antibodies naturally devoid of light chain; VHHs derived from such other species are within the scope of the disclosure. In some embodiments, the single domain antibody (e.g., VHH) provided herein has a structure of FR1-CDR1-FR2-CDR2-FR3-CDR3-FR4. Single domain antibodies may be genetically fused or chemically conjugated to another molecule (e.g., an agent) as described herein.

The term “VHH domain” or “VHH” refers to a single monomeric variable antibody domain that is able to bind selectively to a specific antigen. A VHH domain includes a heavy chain variable antibody fragment having about 110 to about 130 amino acids in length. A VHH domain is also known in the art as a single domain antibody or a nanobody. A VHH domain can include a variable domain of an antibody heavy chain having one or more of the CDR1, CDR2 and CDR3 sequences described herein.

The VHH domains and the molecules comprising a VHH domain and an agent (e.g., therapeutic molecules) described herein (including functional portions and functional variants) can be subject to post-translational modifications. They can be glycosylated, esterified, N-acylated, amidated, carboxylated, phosphorylated, esterified, cyclized via, e.g., a disulfide bridge, or converted into an acid addition salt. In some embodiments, they are dimerized or polymerized, or conjugated.

The VHH domains or molecules comprising a VHH domain and an agent (e.g., therapeutic molecules) described herein (including functional portions and functional variants thereof) can be obtained by methods known in the art. Suitable methods of de novo synthesizing polypeptides and proteins are described in references, such as Chan et al., Fmoc Solid Phase Peptide Synthesis, Oxford University Press, Oxford, United Kingdom, 2000; Peptide and Protein Drug Analysis, ed. Reid, R., Marcel Dekker, Inc., 2000; and Epitope Mapping, ed. Westwood et al., Oxford University Press, Oxford, United Kingdom, 2001. Also, polypeptides and proteins can be recombinantly produced using the nucleic acids described herein using standard recombinant methods. See, for instance, Sambrook et al., Molecular Cloning: A Laboratory Manual, 3rd ed., Cold Spring Harbor Press, Cold Spring Harbor, N.Y. 2001; and Ausubel et al., Current Protocols in Molecular Biology, Greene Publishing Associates and John Wiley & Sons, N Y, 1994. Alternatively, the VHH domains and molecules comprising a VHH domain and an agent (e.g., therapeutic molecules) described herein (including functional portions and functional variants thereof) can be commercially synthesized. In this respect, the VHH domains and molecules comprising a VHH domain and an agent (e.g., therapeutic molecules) described herein can be synthetic, recombinant, isolated, and/or purified.

The nucleic acid can comprise any isolated or purified nucleotide sequence which encodes any of the VHH domains and molecules comprising a VHH domain and an agent (e.g., therapeutic molecules) described herein, or functional portions or functional variants thereof. Alternatively, the nucleotide sequence can comprise a nucleotide sequence which is degenerate to any of the sequences or a combination of degenerate sequences.

Some embodiments provide an isolated or purified nucleic acid comprising a nucleotide sequence which is complementary to the nucleotide sequence of any of the nucleic acids described herein or a nucleotide sequence which hybridizes under stringent conditions to the nucleotide sequence of any of the nucleic acids described herein.

The VHH domains may be conjugated to antibodies or fragments thereof. The antibodies include immunoglobulin molecules including monoclonal antibodies including murine, human, humanized and chimeric monoclonal antibodies, polyclonal, antigen-binding fragments, bispecific or multispecific antibodies, monomeric, dimeric, tetrameric or multimeric antibodies, single chain antibodies, domain antibodies and any other modified configuration of the immunoglobulin molecule that comprises an antigen binding site of the required specificity. The antibody can be a naturally-occurring antibody, e.g., an antibody isolated and/or purified from a mammal, e.g., a murine, primate, mouse, rabbit, goat, horse, chicken, hamster, human, etc. Alternatively, the antibody can be an engineered (e.g., genetically-engineered) antibody.

Humanized antibodies have antigen binding sites derived from non-human species and the variable region frameworks are derived from human immunoglobulin sequences. Human antibodies have heavy and light chain variable regions in which both the framework and the antigen binding site are derived from sequences of human origin.

Suitable methods of making antibodies are known in the art. For instance, standard hybridoma methods are described in, e.g., Köhler and Milstein, Eur. J. Immunol., 5, 511-519 (1976), Harlow and Lane (eds.), Antibodies: A Laboratory Manual, CSH Press (1988), and C. A. Janeway et al. (eds.), Immunobiology, 5th Ed., Garland Publishing, New York, N.Y. (2001)). Alternatively, other methods, such as EBV-hybridoma methods (Haskard and Archer, J. Immunol. Methods, 74(2), 361-67 (1984), and Roder et al., Methods Enzymol., 121, 140-67 (1986)), and bacteriophage vector expression systems (see, e.g., Huse et al., Science, 246, 1275-81 (1989)) are known in the art. Further, methods of producing antibodies in non-human animals are described in, e.g., U.S. Pat. Nos. 5,545,806, 5,569,825, and 5,714,352, and U.S. Patent Application Publication No. 2002/0197266 A1).

Phage display can also be used to generate an antibody. In this regard, phage libraries encoding antigen-binding variable (V) domains of antibodies can be generated using standard molecular biology and recombinant DNA techniques (see, e.g., Sambrook et al., supra, and Ausubel et al., supra). Phage encoding a variable region with the desired specificity are selected for specific binding to the desired antigen, and a complete or partial antibody is reconstituted comprising the selected variable domain. Nucleic acid sequences encoding the reconstituted antibody are introduced into a suitable cell line, such as a myeloma cell used for hybridoma production, such that antibodies having the characteristics of monoclonal antibodies are secreted by the cell (see, e.g., Janeway et al., supra, Huse et al., supra, and U.S. Pat. No. 6,265,150).

Antibodies can be produced by transgenic mice that are transgenic for specific heavy and light chain immunoglobulin genes. Such methods are known in the art and described in, for example U.S. Pat. Nos. 5,545,806 and 5,569,825, and Janeway et al., supra.

In some embodiments, VHH domains provided herein can be humanized VHH domains that bind pIgR, including human pIgR. For example, humanized VHH domains of the present disclosure may comprise one or more CDRs of VHH1, VHH2, VHH3, VHH4, VHH5, VHH6, VHH7, VHH9, VHH10, VHH11 and/or VHH12.

General strategies to humanize single domain antibodies from Camelidae species have been described (see, e.g., Vincke et al., J. Biol. Chem., 2009, 284(5):3273-3284) and are useful for producing humanized VHH domains as disclosed herein.

Humanized antibodies, including humanized VHH domains, can be produced using a variety techniques known in the art, including, but not limited to, methods described in, for example, Janeway et al., supra, U.S. Pat. Nos. 5,225,539, 5,585,089 and 5,693,761, European Patent No. 0239400 Bi, and United Kingdom Patent No. 2188638, CDR-grafting (European Patent No. EP 239,400; International publication No. WO 91/09967; and U.S. Pat. Nos. 5,225,539, 5,530,101, and 5,585,089), veneering or resurfacing (European Patent Nos. EP 592,106 and EP 519,596; Padlan, 1991, Molecular Immunology 28(4/5):489-498; Studnicka et al., 1994, Protein Engineering 7(6):805-814; Roguska et al., 1994, PNAS 91:969-973; and U.S. Pat. No. 5,639,641), chain shuffling (U.S. Pat. No. 5,565,332), and techniques disclosed in, e.g., U.S. Pat. Nos. 6,407,213, 5,766,886, WO 9317105, Tan et al., J. Immunol. 169:1119 25 (2002), Caldas et al., Protein Eng. 13(5):353-60 (2000), Morea et al., Methods 20(3):267 79 (2000), Baca et al., J. Biol. Chem. 272(16):10678-84 (1997), Roguska et al., Protein Eng. 9(10):895 904 (1996), Couto et al., Cancer Res. 55 (23 Supp):5973s-5977s (1995), Couto et al., Cancer Res. 55(8):1717-22 (1995), Sandhu J S, Gene 150(2):409-10 (1994), and Pedersen et al., J. Mol. Biol. 235(3):959-73 (1994). See also U.S. Patent Pub. No. US 2005/0042664 A1 (Feb. 24, 2005), each of which is incorporated by reference herein in its entirety.

In some cases, the humanized antibody is constructed by CDR grafting, in which the amino acid sequences of the CDRs of the parent non-human antibody are grafted onto a human antibody framework. For example, Padlan et al. determined that only about one third of the residues in the CDRs actually contact the antigen, and termed these the “specificity determining residues,” or SDRs (Padlan et al., 1995, FASEB J. 9:133-39). In the technique of SDR grafting, only the SDR residues are grafted onto the human antibody framework (see, e.g., Kashmiri et al., 2005, Methods 36:25-34).

The choice of human variable domains to be used in making the humanized antibodies can be important to reduce antigenicity. For example, according to the so-called “best-fit” method, the sequence of the variable domain of a non-human antibody is screened against the entire library of known human variable-domain sequences. The human sequence that is closest to that of the non-human antibody may be selected as the human framework for the humanized antibody (Sims et al., 1993, J. Immunol. 151:2296-308; and Chothia et al., 1987, J. Mol. Biol. 196:901-17). Another method uses a particular framework derived from the consensus sequence of all human antibodies of a particular subgroup of light or heavy chains. The same framework may be used for several different humanized antibodies (Carter et al., 1992, Proc. Natl. Acad. Sci. USA 89:4285-89; and Presta et al., 1993, J. Immunol. 151:2623-32). In some cases, the framework is derived from the consensus sequences of the most abundant human subclasses, V_{L}6 subgroup I (V_{L}6I) and V_{H} subgroup III (V_{H}III). In another method, human germline genes are used as the source of the framework regions.

In an alternative paradigm based on comparison of CDRs, called superhumanization, framework homology is irrelevant. The method consists of comparison of the non-human sequence with the functional human germline gene repertoire. Those genes encoding the same or closely related canonical structures to the murine sequences are then selected. Next, within the genes sharing the canonical structures with the non-human antibody, those with highest homology within the CDRs are chosen as framework donors. Finally, the non-human CDRs are grafted onto these frameworks (see, e.g., Tan et al., 2002, J. Immunol. 169:1119-25).

It is further generally desirable that VHH domains be humanized with retention of their affinity for the antigen and other favorable biological properties. To achieve this goal, according to one method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. These include, for example, WAM (Whitelegg and Rees, 2000, Protein Eng. 13:819-24), Modeller (Sali and Blundell, 1993, J. Mol. Biol. 234:779-815), and Swiss PDB Viewer (Guex and Peitsch, 1997, Electrophoresis 18:2714-23). Inspection of these displays permits analysis of the likely role of the residues in the functioning of the candidate immunoglobulin sequence, e.g., the analysis of residues that influence the ability of the candidate immunoglobulin to bind its antigen. In this way, framework residues can be selected and combined from the recipient and import sequences so that the desired antibody characteristic, such as increased affinity for the target antigen(s), is achieved. In general, the hypervariable region residues are directly and most substantially involved in influencing antigen binding.

Another method for antibody humanization is based on a metric of antibody humanness termed Human String Content (HSC). This method compares the mouse sequence with the repertoire of human germline genes, and the differences are scored as HSC. The target sequence is then humanized by maximizing its HSC rather than using a global identity measure to generate multiple diverse humanized variants (Lazar et al., 2007, Mol. Immunol. 44:1986-98).

In addition to the methods described above, empirical methods may be used to generate and select humanized antibodies. These methods include those that are based upon the generation of large libraries of humanized variants and selection of the best clones using enrichment technologies or high throughput screening techniques. Antibody variants may be isolated from phage, ribosome, and yeast display libraries as well as by bacterial colony screening (see, e.g., Hoogenboom, 2005, Nat. Biotechnol. 23:1105-16; Dufner et al., 2006, Trends Biotechnol. 24:523-29; Feldhaus et al., 2003, Nat. Biotechnol. 21:163-70; and Schlapschy et al., 2004, Protein Eng. Des. Sel. 17:847-60).

In the framework library approach, a collection of residue variants are introduced at specific positions in the framework followed by screening of the library to select the framework that best supports the grafted CDR. The residues to be substituted may include some or all of the “Vernier” residues identified as potentially contributing to CDR structure (see, e.g., Foote and Winter, 1992, J. Mol. Biol. 224:487-99), or from the more limited set of target residues identified by Baca et al. (1997, J. Biol. Chem. 272:10678-84).

In framework shuffling, whole frameworks are combined with the non-human CDRs instead of creating combinatorial libraries of selected residue variants (see, e.g., Dall'Acqua et al., 2005, Methods 36:43-60). A one-step framework shuffling process may be used. Such a process has been shown to be efficient, as the resulting antibodies exhibited improved biochemical and physicochemical properties including enhanced expression, increased affinity, and thermal stability (see, e.g., Damschroder et al., 2007, Mol. Immunol. 44:3049-60).

The humaneering method is based on experimental identification of essential minimum specificity determinants (MSDs) and is based on sequential replacement of non-human fragments into libraries of human FRs and assessment of binding. This methodology typically results in epitope retention and identification of antibodies from multiple subclasses with distinct human V-segment CDRs.

The human engineering method involves altering a non-human antibody or antibody fragment by making specific changes to the amino acid sequence of the antibody so as to produce a modified antibody with reduced immunogenicity in a human that nonetheless retains the desirable binding properties of the original non-human antibodies. Generally, the technique involves classifying amino acid residues of a non-human antibody as “low risk,” “moderate risk,” or “high risk” residues. The classification is performed using a global risk/reward calculation that evaluates the predicted benefits of making particular substitution (e.g., for immunogenicity in humans) against the risk that the substitution will affect the resulting antibody's folding. The particular human amino acid residue to be substituted at a given position (e.g., low or moderate risk) of a non-human antibody sequence can be selected by aligning an amino acid sequence from the non-human antibody's variable regions with the corresponding region of a specific or consensus human antibody sequence. The amino acid residues at low or moderate risk positions in the non-human sequence can be substituted for the corresponding residues in the human antibody sequence according to the alignment. Techniques for making human engineered proteins are described in greater detail in Studnicka et al., 1994, Protein Engineering 7:805-14; U.S. Pat. Nos. 5,766,886; 5,770,196; 5,821,123; and 5,869,619; and PCT Publication WO 93/11794.

A composite human antibody can be generated using, for example, Composite Human Antibody™ technology (Antitope Ltd., Cambridge, United Kingdom). To generate composite human antibodies, variable region sequences are designed from fragments of multiple human antibody variable region sequences in a manner that avoids T cell epitopes, thereby minimizing the immunogenicity of the resulting antibody.

A deimmunized antibody is an antibody in which T-cell epitopes have been removed. Methods for making deimmunized antibodies have been described. See, e.g., Jones et al., Methods Mol Biol. 2009; 525:405-23, xiv, and De Groot et al., Cell. Immunol. 244:148-153(2006)). Deimmunized antibodies comprise T-cell epitope-depleted variable regions and human constant regions. Briefly, variable regions of an antibody are cloned and T-cell epitopes are subsequently identified by testing overlapping peptides derived from the variable regions of the antibody in a T cell proliferation assay. T cell epitopes are identified via in silico methods to identify peptide binding to human MHC class II. Mutations are introduced in the variable regions to abrogate binding to human MHC class II. Mutated variable regions are then utilized to generate the deimmunized antibody.

Antibodies, as utilized herein, can be multiple or single chain, or intact immunoglobulins, and may be derived from natural sources or from recombinant sources. Antibodies can be tetramers of immunoglobulin molecules.

“Complementarity determining regions (CDR)” are antigen binding sites in a VHH domain. There can be three CDRs (CDR1, CDR2, CDR3) in the VHH domain. The CDRs may be defined by any of the methods described herein, including Kabat, Chothia and IMGT, unless otherwise explicitly stated in the specification.

Also, the molecule comprising a VHH domain, can be modified to comprise a detectable label, such as, for instance, a radioisotope, a fluorophore (e.g., fluorescein isothiocyanate (FITC), phycoerythrin (PE)), an enzyme (e.g., alkaline phosphatase, horseradish peroxidase), and element particles (e.g., gold particles).

Also provided by the present disclosure is a nucleic acid comprising a nucleotide sequence encoding any of the molecules comprising a VHH domain, polypeptides, or proteins described herein (including functional portions and functional variants thereof).

The portion of the VHH domain-containing molecule comprising an antibody or antibody fragment thereof may exist in a variety of forms where the antigen binding domain is expressed as part of a contiguous polypeptide chain including, for example, a single domain antibody fragment (sdAb), a scFv and a human chimeric or humanized antibody (Harlow et al., 1999, In: Using Antibodies: A Laboratory Manual, Cold Spring Harbor Laboratory Press, N.Y.; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426). In one aspect, the antigen binding domain of a VHH molecule of the invention comprises an antibody fragment. In one aspect, the VHH molecule comprises an antibody fragment that comprises a scFv.

The term “antigen” refers to a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is apparent that the present disclosure includes, but is not limited to, the use of partial nucleotide sequences of more than one gene and that these nucleotide sequences are arranged in various combinations to encode polypeptides that elicit the desired immune response.

Without wishing to be bound by theory, the pIgR is responsible for transcytosis of soluble polymeric IgA and IgM, but not IgG, into the mucosal lumen. Though IgG molecules lack a lumen-targeted active transport mechanism, conferring pIgR-binding abilities to IgG can mediate selective transport of IgG antibodies into the mucosal lumen. Anti-pIgR binding VHH antibodies may be effective as a trans-epithelial delivery moiety for the transport of small molecules, proteins, polynucleotides, and other biotherapeutics. Described herein are experiments showing that a panel of pIgR-binding VHH molecules exhibited varying degrees of affinity, species cross-reactivity, biophysical characteristics, epitope diversity, IgA competition profiles and transcytosis activity in a human lung tissue model.

Pharmaceutical compositions of the present disclosure may be administered in a manner appropriate to the disease to be treated (or prevented). The quantity and frequency of administration will be determined by such factors as the condition of the subject, and the type and severity of the subject's disease, although appropriate dosages may be determined by clinical trials. When a therapeutically effective amount is indicated, the precise amount of the compositions of the present disclosure to be administered can be determined by a physician with consideration of individual differences in age, weight, tumor size, extent of infection or metastasis, and condition of the subject.

Many types of release delivery systems are available and known to those of ordinary skill in the art. They include polymer base systems such as poly(lactide-glycolide), copolyoxalates, polyesteramides, polyorthoesters, polycaprolactones, polyhydroxybutyric acid, and polyanhydrides. Microcapsules of the foregoing polymers containing drugs are described in, for example, U.S. Pat. No. 5,075,109. Delivery systems also include non-polymer systems that are lipids including sterols such as cholesterol, cholesterol esters, and fatty acids or neutral fats such as mono-di- and tri-glycerides; sylastic systems; peptide based systems; hydrogel release systems; wax coatings; compressed tablets using conventional binders and excipients; partially fused implants; and the like. Specific examples include, but are not limited to: (a) erosional systems in which the active composition is contained in a form within a matrix such as those described in U.S. Pat. Nos. 4,452,775; 4,667,014; 4,748,034; and 5,239,660 and (b) diffusional systems in which an active component permeates at a controlled rate from a polymer such as described in U.S. Pat. Nos. 3,854,480 and 3,832,253. In addition, pump-based hardware delivery systems can be used, some of which are adapted for implantation.

The administration of the VHH molecules and pharmaceutical compositions may be carried out in any manner, e.g., by parenteral or nonparenteral administration, including by aerosol inhalation, injection, infusions, ingestion, transfusion, implantation or transplantation. For example, the VHH molecules and pharmaceutical compositions described herein may be administered to a patient trans-arterially, intradermally, subcutaneously, intratumorally, intramedullary, intranodally, intramuscularly, by intravenous (i.v.) injection, or intraperitoneally. In one aspect, the compositions of the present disclosure are administered by i.v. injection. In one aspect, the compositions of the present disclosure are administered to a subject by intradermal or subcutaneous injection.

The dosage administered to a patient having a malignancy is sufficient to alleviate or at least partially arrest the disease being treated (“therapeutically effective amount”). The dosage of the above treatments to be administered to a subject will vary with the precise nature of the condition being treated and the recipient of the treatment. The scaling of dosages for human administration can be performed according to practices generally accepted in the art.

Administration may be repeated after one day, two days, three days, four days, five days, six days, one week, two weeks, three weeks, one month, five weeks, six weeks, seven weeks, two months, three months, four months, five months, six months or longer. Repeated courses of treatment are also possible, as is chronic administration. The repeated administration may be at the same dose or at a different dose.

In various embodiments, the VHH molecules described herein are genetically conjugated (e.g., via a linker described herein) to an immunomodulator. Examples of immunomodulators useful herein include, but are not limited to, e.g., afutuzumab (available from Roche®); pegfilgrastim (Neulasta®); lenalidomide (CC-5013, Revlimid®); thalidomide (Thalomid®), actimid (CC4047); and IRX-2 (mixture of human cytokines including interleukin 1, interleukin 2, and interferon-γ, CAS 951209-71-5, available from IRX Therapeutics).

As demonstrated by the present disclosure, the single domain antibodies (e.g., VHH domains) provided herein are useful for transporting an agent from an apical surface of a pIgR-expressing cell to a basolateral surface of the pIgR-expressing cell, and can deliver the agent, e.g., to systemic circulation or Lamina propria or gastrointestinal tract of a subject, via methods such as oral delivery, buccal delivery, nasal delivery or inhalation delivery. In a specific embodiment, the single domain antibody is VHH1 or a VHH having the same CDRs as VHH1. In another specific embodiment, the single domain antibody is VHH2 or a VHH having the same CDRs as VHH2. In another specific embodiment, the single domain antibody is VHH3 or a VHH having the same CDRs as VHH3. In yet another specific embodiment, the single domain antibody is VHH4 or a VHH having the same CDRs as VHH4. In yet another specific embodiment, the single domain antibody is VHH5 or a VHH having the same CDRs as VHH5. In yet another specific embodiment, the single domain antibody is VHH6 or a VHH having the same CDRs as VHH6. In yet another specific embodiment, the single domain antibody is VHH7 or a VHH having the same CDRs as VHH7. In yet another specific embodiment, the single domain antibody is VHH9 or a VHH having the same CDRs as VHH9. In yet another specific embodiment, the single domain antibody is VHH10 or a VHH having the same CDRs as VHH10. In yet another specific embodiment, the single domain antibody is VHH11 or a VHH having the same CDRs as VHH11. In yet another specific embodiment, the single domain antibody is VHH12 or a VHH having the same CDRs as VHH12.

Thus, in some embodiments, provided herein is a method for delivering from an apical surface of a pIgR-expressing cell to a basolateral surface of the pIgR-expressing cell comprising contacting the pIgR-expressing cell with (i) a single domain antibody that binds to pIgR provided herein, or (ii) a therapeutic molecule comprising an agent and the single domain antibody. In a specific embodiment, the single domain antibody is VHH1 or a VHH having the same CDRs as VHH1. In another specific embodiment, the single domain antibody is VHH2 or a VHH having the same CDRs as VHH2. In another specific embodiment, the single domain antibody is VHH3 or a VHH having the same CDRs as VHH3. In yet another specific embodiment, the single domain antibody is VHH4 or a VHH having the same CDRs as VHH4. In yet another specific embodiment, the single domain antibody is VHH5 or a VHH having the same CDRs as VHH5. In yet another specific embodiment, the single domain antibody is VHH6 or a VHH having the same CDRs as VHH6. In yet another specific embodiment, the single domain antibody is VHH7 or a VHH having the same CDRs as VHH7. In yet another specific embodiment, the single domain antibody is VHH9 or a VHH having the same CDRs as VHH9. In yet another specific embodiment, the single domain antibody is VHH10 or a VHH having the same CDRs as VHH10. In yet another specific embodiment, the single domain antibody is VHH11 or a VHH having the same CDRs as VHH11. In yet another specific embodiment, the single domain antibody is VHH12 or a VHH having the same CDRs as VHH12.

In some embodiments, provide herein is a single domain antibody that binds to pIgR provided herein for use in delivering an agent from an apical surface of a pIgR-expressing cell to a basolateral surface of the pIgR-expressing cell, wherein the agent is conjugated to the single domain antibody. In a specific embodiment, the single domain antibody is VHH1 or a VHH having the same CDRs as VHH1. In another specific embodiment, the single domain antibody is VHH2 or a VHH having the same CDRs as VHH2. In another specific embodiment, the single domain antibody is VHH3 or a VHH having the same CDRs as VHH3. In yet another specific embodiment, the single domain antibody is VHH4 or a VHH having the same CDRs as VHH4. In yet another specific embodiment, the single domain antibody is VHH5 or a VHH having the same CDRs as VHH5. In yet another specific embodiment, the single domain antibody is VHH6 or a VHH having the same CDRs as VHH6. In yet another specific embodiment, the single domain antibody is VHH7 or a VHH having the same CDRs as VHH7. In yet another specific embodiment, the single domain antibody is VHH9 or a VHH having the same CDRs as VHH9. In yet another specific embodiment, the single domain antibody is VHH10 or a VHH having the same CDRs as VHH10. In yet another specific embodiment, the single domain antibody is VHH11 or a VHH having the same CDRs as VHH11. In yet another specific embodiment, the single domain antibody is VHH12 or a VHH having the same CDRs as VHH12.

In some embodiments, provided herein is a use of a single domain antibody that binds to pIgR provided herein for delivering an agent from an apical surface of a pIgR-expressing cell to a basolateral surface of the pIgR-expressing cell, wherein the agent is conjugated to the single domain antibody. In a specific embodiment, the single domain antibody is VHH1 or a VHH having the same CDRs as VHH1. In another specific embodiment, the single domain antibody is VHH2 or a VHH having the same CDRs as VHH2. In another specific embodiment, the single domain antibody is VHH3 or a VHH having the same CDRs as VHH3. In yet another specific embodiment, the single domain antibody is VHH4 or a VHH having the same CDRs as VHH4. In yet another specific embodiment, the single domain antibody is VHH5 or a VHH having the same CDRs as VHH5. In yet another specific embodiment, the single domain antibody is VHH6 or a VHH having the same CDRs as VHH6. In yet another specific embodiment, the single domain antibody is VHH7 or a VHH having the same CDRs as VHH7. In yet another specific embodiment, the single domain antibody is VHH9 or a VHH having the same CDRs as VHH9. In yet another specific embodiment, the single domain antibody is VHH10 or a VHH having the same CDRs as VHH10. In yet another specific embodiment, the single domain antibody is VHH11 or a VHH having the same CDRs as VHH11. In yet another specific embodiment, the single domain antibody is VHH12 or a VHH having the same CDRs as VHH12.

In other embodiments, provided herein is a method for transporting a therapeutic molecule to a basolateral surface of the pIgR-expressing cell of a subject, comprising administering to the subject the therapeutic molecule comprising an agent and a VHH domain. In some embodiments, the therapeutic molecule is administered to the subject via oral delivery, buccal delivery, nasal delivery or inhalation delivery. In a specific embodiment, the single domain antibody is VHH1 or a VHH having the same CDRs as VHH1. In another specific embodiment, the single domain antibody is VHH2 or a VHH having the same CDRs as VHH2. In another specific embodiment, the single domain antibody is VHH3 or a VHH having the same CDRs as VHH3. In yet another specific embodiment, the single domain antibody is VHH4 or a VHH having the same CDRs as VHH4. In yet another specific embodiment, the single domain antibody is VHH5 or a VHH having the same CDRs as VHH5. In yet another specific embodiment, the single domain antibody is VHH6 or a VHH having the same CDRs as VHH6. In yet another specific embodiment, the single domain antibody is VHH7 or a VHH having the same CDRs as VHH7. In yet another specific embodiment, the single domain antibody is VHH9 or a VHH having the same CDRs as VHH9. In yet another specific embodiment, the single domain antibody is VHH10 or a VHH having the same CDRs as VHH10. In yet another specific embodiment, the single domain antibody is VHH11 or a VHH having the same CDRs as VHH11. In yet another specific embodiment, the single domain antibody is VHH12 or a VHH having the same CDRs as VHH12.

In other embodiments, provided herein is a single domain antibody for use in transporting a therapeutic molecule to a basolateral surface of the pIgR-expressing cell of a subject, wherein the therapeutic molecule comprises an agent and the single domain antibody. In some embodiments, the therapeutic molecule is administered to the subject via oral delivery, buccal delivery, nasal delivery or inhalation delivery. In a specific embodiment, the single domain antibody is VHH1 or a VHH having the same CDRs as VHH1. In another specific embodiment, the single domain antibody is VHH2 or a VHH having the same CDRs as VHH2. In another specific embodiment, the single domain antibody is VHH3 or a VHH having the same CDRs as VHH3. In yet another specific embodiment, the single domain antibody is VHH4 or a VHH having the same CDRs as VHH4. In yet another specific embodiment, the single domain antibody is VHH5 or a VHH having the same CDRs as VHH5. In yet another specific embodiment, the single domain antibody is VHH6 or a VHH having the same CDRs as VHH6. In yet another specific embodiment, the single domain antibody is VHH7 or a VHH having the same CDRs as VHH7. In yet another specific embodiment, the single domain antibody is VHH9 or a VHH having the same CDRs as VHH9. In yet another specific embodiment, the single domain antibody is VHH10 or a VHH having the same CDRs as VHH10. In yet another specific embodiment, the single domain antibody is VHH11 or a VHH having the same CDRs as VHH11. In yet another specific embodiment, the single domain antibody is VHH12 or a VHH having the same CDRs as VHH12.

In other embodiments, provided herein is a use of a single domain antibody for transporting a therapeutic molecule to a basolateral surface of the pIgR-expressing cell of a subject, wherein the therapeutic molecule comprises an agent and the single domain antibody. In some embodiments, the therapeutic molecule is administered to the subject via oral delivery, buccal delivery, nasal delivery or inhalation delivery. In a specific embodiment, the single domain antibody is VHH1 or a VHH having the same CDRs as VHH1. In another specific embodiment, the single domain antibody is VHH2 or a VHH having the same CDRs as VHH2. In another specific embodiment, the single domain antibody is VHH3 or a VHH having the same CDRs as VHH3. In yet another specific embodiment, the single domain antibody is VHH4 or a VHH having the same CDRs as VHH4. In yet another specific embodiment, the single domain antibody is VHH5 or a VHH having the same CDRs as VHH5. In yet another specific embodiment, the single domain antibody is VHH6 or a VHH having the same CDRs as VHH6. In yet another specific embodiment, the single domain antibody is VHH7 or a VHH having the same CDRs as VHH7. In yet another specific embodiment, the single domain antibody is VHH9 or a VHH having the same CDRs as VHH9. In yet another specific embodiment, the single domain antibody is VHH10 or a VHH having the same CDRs as VHH10. In yet another specific embodiment, the single domain antibody is VHH11 or a VHH having the same CDRs as VHH11. In yet another specific embodiment, the single domain antibody is VHH12 or a VHH having the same CDRs as VHH12.

In yet other embodiments, provided herein is a method for transporting a therapeutic molecule to systemic circulation of a subject, comprising administering to the subject the therapeutic molecule comprising an agent and a single domain antibody, wherein the therapeutic molecule is administered to the subject via oral delivery, buccal delivery, nasal delivery or inhalation delivery. In a specific embodiment, the single domain antibody is VHH1 or a VHH having the same CDRs as VHH1. In another specific embodiment, the single domain antibody is VHH2 or a VHH having the same CDRs as VHH2. In another specific embodiment, the single domain antibody is VHH3 or a VHH having the same CDRs as VHH3. In yet another specific embodiment, the single domain antibody is VHH4 or a VHH having the same CDRs as VHH4. In yet another specific embodiment, the single domain antibody is VHH5 or a VHH having the same CDRs as VHH5. In yet another specific embodiment, the single domain antibody is VHH6 or a VHH having the same CDRs as VHH6. In yet another specific embodiment, the single domain antibody is VHH7 or a VHH having the same CDRs as VHH7. In yet another specific embodiment, the single domain antibody is VHH9 or a VHH having the same CDRs as VHH9. In yet another specific embodiment, the single domain antibody is VHH10 or a VHH having the same CDRs as VHH10. In yet another specific embodiment, the single domain antibody is VHH11 or a VHH having the same CDRs as VHH11. In yet another specific embodiment, the single domain antibody is VHH12 or a VHH having the same CDRs as VHH12.

In yet other embodiments, provided herein is a single domain antibody for use in transporting a therapeutic molecule to systemic circulation of a subject, wherein the therapeutic molecule comprises the single domain antibody and an agent, and wherein the therapeutic molecule is administered to the subject via oral delivery, buccal delivery, nasal delivery or inhalation delivery. In a specific embodiment, the single domain antibody is VHH1 or a VHH having the same CDRs as VHH1. In another specific embodiment, the single domain antibody is VHH2 or a VHH having the same CDRs as VHH2. In another specific embodiment, the single domain antibody is VHH3 or a VHH having the same CDRs as VHH3. In yet another specific embodiment, the single domain antibody is VHH4 or a VHH having the same CDRs as VHH4. In yet another specific embodiment, the single domain antibody is VHH5 or a VHH having the same CDRs as VHH5. In yet another specific embodiment, the single domain antibody is VHH6 or a VHH having the same CDRs as VHH6. In yet another specific embodiment, the single domain antibody is VHH7 or a VHH having the same CDRs as VHH7. In yet another specific embodiment, the single domain antibody is VHH9 or a VHH having the same CDRs as VHH9. In yet another specific embodiment, the single domain antibody is VHH10 or a VHH having the same CDRs as VHH10. In yet another specific embodiment, the single domain antibody is VHH11 or a VHH having the same CDRs as VHH11. In yet another specific embodiment, the single domain antibody is VHH12 or a VHH having the same CDRs as VHH12.

In yet other embodiments, provided herein is a use of VHH for transporting a therapeutic molecule to systemic circulation of a subject, wherein the therapeutic molecule comprises the single domain antibody and an agent, and wherein the therapeutic molecule is administered to the subject via oral delivery, buccal delivery, nasal delivery or inhalation delivery.

In yet other embodiments, provided herein is a method for transporting a therapeutic molecule to Lamina propria or gastrointestinal tract of a subject, comprising administering to the subject the therapeutic molecule comprising an agent and a single domain antibody, wherein the therapeutic molecule is administered to the subject via oral delivery, buccal delivery, nasal delivery or inhalation delivery. In a specific embodiment, the single domain antibody is VHH1 or a VHH having the same CDRs as VHH1. In another specific embodiment, the single domain antibody is VHH2 or a VHH having the same CDRs as VHH2. In another specific embodiment, the single domain antibody is VHH3 or a VHH having the same CDRs as VHH3. In yet another specific embodiment, the single domain antibody is VHH4 or a VHH having the same CDRs as VHH4. In yet another specific embodiment, the single domain antibody is VHH5 or a VHH having the same CDRs as VHH5. In yet another specific embodiment, the single domain antibody is VHH6 or a VHH having the same CDRs as VHH6. In yet another specific embodiment, the single domain antibody is VHH7 or a VHH having the same CDRs as VHH7. In yet another specific embodiment, the single domain antibody is VHH9 or a VHH having the same CDRs as VHH9. In yet another specific embodiment, the single domain antibody is VHH10 or a VHH having the same CDRs as VHH10. In yet another specific embodiment, the single domain antibody is VHH11 or a VHH having the same CDRs as VHH11. In yet another specific embodiment, the single domain antibody is VHH12 or a VHH having the same CDRs as VHH12.

In yet other embodiments, provided herein is a single domain antibody for use in transporting a therapeutic molecule to Lamina propria or gastrointestinal tract of a subject, wherein the therapeutic molecule comprises an agent and the single domain antibody, and wherein the therapeutic molecule is administered to the subject via oral delivery, buccal delivery, nasal delivery or inhalation delivery. In a specific embodiment, the single domain antibody is VHH1 or a VHH having the same CDRs as VHH1. In another specific embodiment, the single domain antibody is VHH2 or a VHH having the same CDRs as VHH2. In another specific embodiment, the single domain antibody is VHH3 or a VHH having the same CDRs as VHH3. In yet another specific embodiment, the single domain antibody is VHH4 or a VHH having the same CDRs as VHH4. In yet another specific embodiment, the single domain antibody is VHH5 or a VHH having the same CDRs as VHH5. In yet another specific embodiment, the single domain antibody is VHH6 or a VHH having the same CDRs as VHH6. In yet another specific embodiment, the single domain antibody is VHH7 or a VHH having the same CDRs as VHH7. In yet another specific embodiment, the single domain antibody is VHH9 or a VHH having the same CDRs as VHH9. In yet another specific embodiment, the single domain antibody is VHH10 or a VHH having the same CDRs as VHH10. In yet another specific embodiment, the single domain antibody is VHH11 or a VHH having the same CDRs as VHH11. In yet another specific embodiment, the single domain antibody is VHH12 or a VHH having the same CDRs as VHH12.

In yet other embodiments, provided herein is a use of a single domain antibody for transporting a therapeutic molecule to Lamina propria or gastrointestinal tract of a subject, wherein the therapeutic molecule comprises an agent and the single domain antibody, and wherein the therapeutic molecule is administered to the subject via oral delivery, buccal delivery, nasal delivery or inhalation delivery. In a specific embodiment, the single domain antibody is VHH1 or a VHH having the same CDRs as VHH1. In another specific embodiment, the single domain antibody is VHH2 or a VHH having the same CDRs as VHH2. In another specific embodiment, the single domain antibody is VHH3 or a VHH having the same CDRs as VHH3. In yet another specific embodiment, the single domain antibody is VHH4 or a VHH having the same CDRs as VHH4. In yet another specific embodiment, the single domain antibody is VHH5 or a VHH having the same CDRs as VHH5. In yet another specific embodiment, the single domain antibody is VHH6 or a VHH having the same CDRs as VHH6. In yet another specific embodiment, the single domain antibody is VHH7 or a VHH having the same CDRs as VHH7. In yet another specific embodiment, the single domain antibody is VHH9 or a VHH having the same CDRs as VHH9. In yet another specific embodiment, the single domain antibody is VHH10 or a VHH having the same CDRs as VHH10. In yet another specific embodiment, the single domain antibody is VHH11 or a VHH having the same CDRs as VHH11. In yet another specific embodiment, the single domain antibody is VHH12 or a VHH having the same CDRs as VHH12.

In some embodiments of the various methods and uses provided herein, the therapeutic agent is transported from an apical surface of a pIgR-expressing cell to a basolateral surface of the pIgR-expressing cell in the subject.

In some embodiments, the single domain antibody or the therapeutic molecule comprising an agent and the single domain antibody is also capable of being transported from the basolateral surface of the pIgR-expressing cell to the apical surface of the pIgR-expressing cell.

In yet other embodiments, provided herein is a method of treating a disease or disorder comprising administering a therapeutic molecule comprising an agent and the single domain antibody provided herein to a subject, wherein optionally the therapeutic molecule is administered to the subject via oral delivery, buccal delivery, nasal delivery or inhalation delivery. In a specific embodiment, the single domain antibody is VHH1 or a VHH having the same CDRs as VHH1. In another specific embodiment, the single domain antibody is VHH2 or a VHH having the same CDRs as VHH2. In another specific embodiment, the single domain antibody is VHH3 or a VHH having the same CDRs as VHH3. In yet another specific embodiment, the single domain antibody is VHH4 or a VHH having the same CDRs as VHH4. In yet another specific embodiment, the single domain antibody is VHH5 or a VHH having the same CDRs as VHH5. In yet another specific embodiment, the single domain antibody is VHH6 or a VHH having the same CDRs as VHH6. In yet another specific embodiment, the single domain antibody is VHH7 or a VHH having the same CDRs as VHH7. In yet another specific embodiment, the single domain antibody is VHH9 or a VHH having the same CDRs as VHH9. In yet another specific embodiment, the single domain antibody is VHH10 or a VHH having the same CDRs as VHH10. In yet another specific embodiment, the single domain antibody is VHH11 or a VHH having the same CDRs as VHH11. In yet another specific embodiment, the single domain antibody is VHH12 or a VHH having the same CDRs as VHH12.

In yet other embodiments, provided herein is a therapeutic molecule comprising an agent and a single domain antibody provided herein for use in treating a disease or disorder in subject, wherein optionally the therapeutic molecule is administered to the subject via oral delivery, buccal delivery, nasal delivery or inhalation delivery. In a specific embodiment, the single domain antibody is VHH1 or a VHH having the same CDRs as VHH1. In another specific embodiment, the single domain antibody is VHH2 or a VHH having the same CDRs as VHH2. In another specific embodiment, the single domain antibody is VHH3 or a VHH having the same CDRs as VHH3. In yet another specific embodiment, the single domain antibody is VHH4 or a VHH having the same CDRs as VHH4. In yet another specific embodiment, the single domain antibody is VHH5 or a VHH having the same CDRs as VHH5. In yet another specific embodiment, the single domain antibody is VHH6 or a VHH having the same CDRs as VHH6. In yet another specific embodiment, the single domain antibody is VHH7 or a VHH having the same CDRs as VHH7. In yet another specific embodiment, the single domain antibody is VHH9 or a VHH having the same CDRs as VHH9. In yet another specific embodiment, the single domain antibody is VHH10 or a VHH having the same CDRs as VHH10. In yet another specific embodiment, the single domain antibody is VHH11 or a VHH having the same CDRs as VHH11. In yet another specific embodiment, the single domain antibody is VHH12 or a VHH having the same CDRs as VHH12.

In yet other embodiments, provided herein is a use of a therapeutic molecule comprising an agent and a single domain antibody provided herein for treating a disease or disorder in subject, wherein optionally the therapeutic molecule is administered to the subject via oral delivery, buccal delivery, nasal delivery or inhalation delivery. In a specific embodiment, the single domain antibody is VHH1 or a VHH having the same CDRs as VHH1. In another specific embodiment, the single domain antibody is VHH2 or a VHH having the same CDRs as VHH2. In another specific embodiment, the single domain antibody is VHH3 or a VHH having the same CDRs as VHH3. In yet another specific embodiment, the single domain antibody is VHH4 or a VHH having the same CDRs as VHH4. In yet another specific embodiment, the single domain antibody is VHH5 or a VHH having the same CDRs as VHH5. In yet another specific embodiment, the single domain antibody is VHH6 or a VHH having the same CDRs as VHH6. In yet another specific embodiment, the single domain antibody is VHH7 or a VHH having the same CDRs as VHH7. In yet another specific embodiment, the single domain antibody is VHH9 or a VHH having the same CDRs as VHH9. In yet another specific embodiment, the single domain antibody is VHH10 or a VHH having the same CDRs as VHH10. In yet another specific embodiment, the single domain antibody is VHH11 or a VHH having the same CDRs as VHH11. In yet another specific embodiment, the single domain antibody is VHH12 or a VHH having the same CDRs as VHH12.

In some embodiments, the disease or disorder is a metabolic disease or disorder. In some embodiments, the disease or disorder is diabetes. In some embodiments, the disease or disorder is cancer. In other embodiments, the disease or disorder is an immune disease or disorder. In some embodiments, the disease or disorder is a gastrointestinal disease. In some embodiments, the disease or disorder is gastrointestinal inflammation. In some embodiments, the disease or disorder is inflammatory bowel disease (IBD). In some embodiments, the disease or disorder is Crohn's disease (CD). In some embodiments, the disease or disorder is ulcerative colitis (UC). In some embodiments, the disease or disorder is ankylosing spondylitis (AS). In some embodiments, the disease or disorder is colitis.

For example, the single domain antibodies of the disclosure may be conjugated to any agent that can be used to treat or ameliorate symptoms of intestinal inflammation, IBD, UC or AS, including agents which are inhibitors of pro-inflammatory cytokines, inhibitors of Th17 cell activation and/or differentiation, molecules inhibiting lymphocyte trafficking or adhesion, modulators of innate immune system, modulators of macrophages, dendritic cells, regulatory T cells (Treg) or effector CD8⁺ or CD4⁺ T cells. Such exemplary agents include inhibitors of TNF-α IL-12, IL-6, IL-13, IL-17 Å, IL17A/F, IL-18, IL-21, modulators of TLR3 or TLR4 pathway, TNF-α inhibitors infliximab, adalimumab, certolizumab, golimumab, etanercept and biosimilars thereof, IL-23 inhibitors ustekinumab, risankizumab, brazikumab and mirikizumab, IL-23 receptor inhibitors, IL-17 inhibitor secukinumab, IL-6 inhibitors tocilizumab and PF-04236921, PDE4 inhibitor apremilast, JAK inhibitors tofacitinib, filgotinib, upadacitinib or peficiting, inhibitors of cell adhesion such as natalizumab, vedolizumab, etrolizumab, abrilumab, PF-00547659, integrin antagonists or sphingosine 1 phosphate receptor modulators, or agents enhancing production of IL-10. In some embodiments, the agent is an inhibitor of IL-23 receptor. The agent targeting pathogenic pathways in intestinal inflammation herein may be a known molecule, a variant or a fragment of the known molecule, or generated de novo and genetically fused or chemically conjugated to the single domain antibody of the disclosure using known methods and those described herein.

In some embodiments, the methods or uses provided here are for delivering a vaccine for preventing an infection, such as Vibrio, Cholera, Typhoid, Rotavirus, Tuberculosis, HIV, Flu, Ebola, and Sendai.

In some embodiments of the various methods and uses provided herein, the agent in the therapeutic molecule comprises a peptide. In some embodiments of the various methods and uses provided herein, the agent in the therapeutic molecule comprises an antibody or a fragment thereof. In some embodiments of the various methods and uses provided herein, the agent in the therapeutic molecule comprises a peptide conjugated to a small molecule compound (e.g., antibody drug conjugate). In some embodiments of the various methods and uses provided herein, the agent in the therapeutic molecule comprises a nucleic acid. In some embodiments of the various methods and uses provided herein, the agent in the therapeutic molecule comprises a vaccine.

The amount of a prophylactic or therapeutic agent (e.g., an antibody or therapeutic molecule), or a composition provided herein that will be effective in the prevention and/or treatment of a disease or condition can be determined by standard clinical techniques. In addition, in vitro assays may optionally be employed to help identify optimal dosage ranges. The precise dose to be employed in the formulation will also depend on the route of administration, and the seriousness of a disease or condition, and should be decided according to the judgment of the practitioner and each patient's circumstances.

Effective doses may be extrapolated from dose-response curves derived from in vitro or animal model test systems. In certain embodiments, the route of administration for a dose of an antibody or therapeutic molecule provided herein to a patient is oral delivery, buccal delivery, nasal delivery, inhalation delivery, or a combination thereof, but other routes may be also acceptable. Each dose may or may not be administered by an identical route of administration. In some embodiments, an antibody or therapeutic molecule provided herein may be administered via multiple routes of administration simultaneously or subsequently to other doses of the same or a different agent provided herein.

A description of example embodiments follows.

1. A method of modulating a function of pIgR in a cell comprising contacting the cell with a molecule comprising a VHH domain, including an effective amount of the molecule, wherein optionally the VHH domain binds to an extracellular domain of the pIgR, including a VHH domain that binds to an extracellular domain 1, an extracellular domain 2, an extracellular domain 1-2, an extracellular domain 3, an extracellular domain 2-3, an extracellular domain 4-5, or an extracellular domain 5 of pIgR, wherein optionally pIgR is human pIgR or mouse pIgR, wherein optionally the VHH domain does not detectably bind to the amino acid sequence of

(SEQ ID NO: 143)
EKAVADTRDQADGSRASVDSGSSEEQGGSSR,
(SEQ ID NO: 144)
EREIQNVGDQAQENRASGDAGSADGQSRSSSSK
or
(SEQ ID NO: 145)
EREIQNVRDQAQENRASGDAGSADGQSRSSSSK.

2. The method of embodiment 1, wherein the modulating the function of pIgR in the cell is activating said function of pIgR in said cell.

3. A method of inhibiting the binding of IgA to pIgR in a cell, the method comprising contacting the cell with a molecule comprising a VHH domain, including an effective amount of the molecule, wherein optionally the VHH domain binds to an extracellular domain of the pIgR, including a VHH domain that binds to an extracellular domain 1, an extracellular domain 2, an extracellular domain 1-2, an extracellular domain 3, an extracellular domain 2-3, an extracellular domain 4-5, or an extracellular domain 5 of pIgR, wherein optionally pIgR is human pIgR or mouse pIgR, wherein optionally the VHH domain does not detectably bind to the amino acid sequence of EKAVADTRDQADGSRASVDSGSSEEQGGSSR (SEQ ID NO: 143), EREIQNVGDQAQENRASGDAGSADGQSRSSSSK (SEQ ID NO: 144) or EREIQNVRDQAQENRASGDAGSADGQSRSSSSK (SEQ ID NO: 145).

4. A method of increasing the rate of pIgR-mediated transcytosis (forward transcytosis or reverse transcytosis) across an epithelial cell comprising contacting the cell with a molecule comprising a VHH domain, including wherein optionally the VHH domain binds to an extracellular domain of the pIgR, including a VHH domain that binds to an extracellular domain 1, an extracellular domain 2, an extracellular domain 1-2, an extracellular domain 3, an extracellular domain 2-3, an extracellular domain 4-5, or an extracellular domain 5 of pIgR, wherein optionally pIgR is human pIgR or mouse pIgR, wherein optionally the VHH domain does not detectably bind to the amino acid sequence of

(SEQ ID NO: 143)
EKAVADTRDQADGSRASVDSGSSEEQGGSSR
or
(SEQ ID NO: 144)
EREIQNVGDQAQENRASGDAGSADGQSRSSSSK.

5. The method of embodiment 4, wherein the epithelial cell is a mucosal epithelial cell.

6. A method of delivering a molecule to a pIgR-expressing cell comprising contacting the cell with said molecule genetically fused or chemically conjugated to a VHH domain, including wherein optionally the VHH domain binds to an extracellular domain of the pIgR, including a VHH domain that binds to an extracellular domain 1, an extracellular domain 2, an extracellular domain 1-2, an extracellular domain 3, an extracellular domain 2-3, an extracellular domain 4-5, or an extracellular domain 5 of pIgR, wherein optionally pIgR is human pIgR or mouse pIgR, wherein optionally the VHH domain does not detectably bind to the amino acid sequence of EKAVADTRDQADGSRASVDSGSSEEQGGSSR (SEQ ID NO: 143), EREIQNVGDQAQENRASGDAGSADGQSRSSSSK (SEQ ID NO: 144) or EREIQNVRDQAQENRASGDAGSADGQSRSSSSK (SEQ ID NO: 145).

7. The method of any one of embodiments 1-6, wherein the cell is a mucosal epithelial cell.

8. The method of any one of embodiments 1-7, wherein the cell is a cancer cell.

9. The method of embodiment 8, wherein the cancer cell is a lung cancer cell, an esophageal cancer cell, a stomach cancer cell, a duodenal cancer cell, a liver cancer cell, a bladder cancer cell, a sinus cancer cell, a nasal cavity cancer cell, an endometrial cancer cell or a colorectal cancer cell.

10. The method of any one of embodiments 1-9, wherein the cell is in a subject.

11. A method of delivering a molecule to a mucosal lumen of a subject, the method comprising administering to the subject a molecule comprising a VHH domain, including wherein optionally the VHH domain binds to an extracellular domain of the pIgR, including a VHH domain that binds to an extracellular domain 1, an extracellular domain 2, an extracellular domain 1-2, an extracellular domain 3, an extracellular domain 2-3, an extracellular domain 4-5, or an extracellular domain 5 of pIgR, wherein optionally pIgR is human pIgR or mouse pIgR, wherein optionally the VHH domain does not detectably bind to the amino acid sequence of

(SEQ ID NO: 143)
EKAVADTRDQADGSRASVDSGSSEEQGGSSR,
(SEQ ID NO: 144)
EREIQNVGDQAQENRASGDAGSADGQSRSSSSK
or
(SEQ ID NO: 145)
EREIQNVRDQAQENRASGDAGSADGQSRSSSSK.

12. The method of embodiment 11, wherein the mucosal lumen is in the lung or in the gastrointestinal tract of the subject.

13. A method of delivering a molecule to an organ of a subject, the method comprising administering to the subject a molecule comprising a VHH domain, including wherein optionally the VHH domain binds to an extracellular domain of the pIgR, including a VHH domain that binds to an extracellular domain 1, an extracellular domain 2, an extracellular domain 1-2, an extracellular domain 3, an extracellular domain 2-3, an extracellular domain 4-5, or an extracellular domain 5 of pIgR, wherein optionally pIgR is human pIgR or mouse pIgR, wherein optionally the VHH domain does not detectably bind to the amino acid sequence of

(SEQ ID NO: 143)
EKAVADTRDQADGSRASVDSGSSEEQGGSSR,
(SEQ ID NO: 144)
EREIQNVGDQAQENRASGDAGSADGQSRSSSSK
or
(SEQ ID NO: 145)
EREIQNVRDQAQENRASGDAGSADGQSRSSSSK.

14. The method of embodiment 13, wherein the organ is selected from the group consisting of small intestine, large intestine, stomach, esophagus, salivary gland, lung, vagina, uterus, and lacrimal gland.

15. The method of embodiment 14, wherein the organ is a lung.

16. The method of any one of embodiments 6-15, wherein the molecule is an antibody or fragment thereof, a peptide, a vaccine, a small molecule, a polynucleotide, a molecule comprising a radioactive isotope, a toxin, an enzyme, an anticoagulant, a hormone, a cytokine, an anti-inflammatory molecule, an RNAi, an mRNA, a self-replicating RNA, an antibiotic, or an antibody-antibiotic conjugate.

17. The method of embodiment 16, wherein the molecule is an antibiotic, an antibody or fragment thereof, a peptide or a vaccine.

18. The method of embodiment 17, wherein the antibiotic is selected from the group consisting of a macrolide antibiotic, a fluoroquinolone, a tetracycline, amoxicillin, ceftriaxone, penicillin G, linezolid, moxifloxacin, and azithromycin.

19. A method of detecting pIgR expressing cells in a subject comprising administering to the subject a molecule comprising a VHH domain, including wherein optionally the VHH domain binds to an extracellular domain of the pIgR, including a VHH domain that binds to an extracellular domain 1, an extracellular domain 2, an extracellular domain 1-2, an extracellular domain 3, an extracellular domain 2-3, an extracellular domain 4-5, or an extracellular domain 5 of pIgR, wherein optionally pIgR is human pIgR or mouse pIgR, wherein optionally the VHH domain does not detectably bind to the amino acid sequence of

(SEQ ID NO: 143)
EKAVADTRDQADGSRASVDSGSSEEQGGSSR,
(SEQ ID NO: 144)
EREIQNVGDQAQENRASGDAGSADGQSRSSSSK
or
(SEQ ID NO: 145)
EREIQNVRDQAQENRASGDAGSADGQSRSSSSK.

20. The method of embodiment 19, wherein the molecule comprising the VHH domain is a radioisotope-labelled VHH-Fc or a radioisotope-labelled VHH-antibody conjugate, optionally wherein the radioisotope is ¹⁸F, ⁹⁹Tc, ¹¹¹In, ¹²³I, ²⁰¹Tl, ¹³³Xe, ¹¹C, ¹³N, ¹⁵O, ¹⁸F, ⁶²Cu, ⁶⁴Cu, ¹²⁴I, ⁷⁶Br, ⁸²Rb, ⁸⁹Zr or ⁶⁸Ga.

21. The method of embodiment 18 or embodiment 19, wherein pIgR expressing cells are mucosal epithelial cells.

22. The method of any one of embodiments 18-20, wherein pIgR expressing cells are cancer cells.

23. The method of embodiment 22, wherein the cancer cell is a lung cancer cell, an esophageal cancer cell, a stomach cancer cell, a duodenal cancer cell, a liver cancer cell, a bladder cancer cell, a sinus cancer cell, a nasal cavity cancer cell, an endometrial cancer cell or a colorectal cancer cell.

24. A method of treating a disease in a subject in need thereof, comprising administering to the subject a molecule comprising a VHH domain, including a therapeutically effective amount of the molecule, wherein optionally the VHH domain binds to an extracellular domain of the pIgR, including a VHH domain that binds to an extracellular domain 1, an extracellular domain 2, an extracellular domain 1-2, an extracellular domain 3, an extracellular domain 2-3, an extracellular domain 4-5, or an extracellular domain 5 of pIgR, wherein optionally pIgR is human pIgR or mouse pIgR, wherein optionally the VHH domain does not detectably bind to the amino acid sequence of

(SEQ ID NO: 143)
EKAVADTRDQADGSRASVDSGSSEEQGGSSR,
(SEQ ID NO: 144)
EREIQNVGDQAQENRASGDAGSADGQSRSSSSK
or
(SEQ ID NO. 145)
EREIQNVRDQAQENRASGDAGSADGQSRSSSSK.

25. The method of embodiment 24, wherein the disease is a cancer, an inflammatory disease, inflammatory bowel disease, pneumonia, cystic fibrosis, lung infection, asthma, tuberculosis, chronic obstructive pulmonary disease (COPD), bronchitis and emphysema, Crohn's disease, ulcerative colitis, cystitis, overactive bladder disease, sinus infection, gastrointestinal ulcer, adenomyosis, uterine inflammation, hepatobiliary disease, or hepatitis.

26. The method of embodiment 24 or embodiment 25, wherein the molecule comprises a VHH domain genetically fused or chemically conjugated to a molecule selected from the group consisting of an antibody or fragment thereof, a peptide, a vaccine, a radioactive isotope, a toxin, an enzyme, an anticoagulant, a hormone, a cytokine, an anti-inflammatory molecule, an RNAi, an mRNA, a self-replicating RNA, an antibiotic, and an antibody-antibiotic conjugate

27. The method of embodiment 24 or embodiment 25, wherein the molecule comprises a VHH domain genetically fused or chemically conjugated to a polypeptide or a polynucleotide.

28. The method of any one of embodiments 10-27, wherein the molecule is administered to the bloodstream of the subject.

29. The method of any one of embodiments 10-28, wherein the molecule is administered intravenously or subcutaneously.

30. The method of any one of embodiments 6 to 29, wherein the molecule further comprises a linker.

31. The method of embodiment 30, wherein the linker is a polypeptide.

32. The method of embodiment 31, wherein the linker is a flexible linker comprising a sequence selected from the group consisting of EPKTPKPQPQPQLQPQPNPTTESKSPK (SEQ ID NO: 130), (EAAAK)n (SEQ ID NO: 147), (GGGGS)n (SEQ ID NO: 148) and (GGGS)n (SEQ ID NO: 149), wherein n is an integer from 1 to 20.

33. The method of any one of embodiments 6 to 33, wherein the VHH domain is chemically-conjugated to the molecule.

34. The method of any one of embodiments 6 to 33, wherein the VHH domain is non-covalently bound to the molecule.

35. The method of any one of embodiments 1 to 34, wherein the VHH domain comprises a complementarity determining region 1 (CDR1) comprising the amino acid sequence of SYRMG (SEQ ID NO: 1), INVMG (SEQ ID NO: 2), SNAMG (SEQ ID NO: 3), SYAMG (SEQ ID NO: 4), SDAMG (SEQ ID NO: 5), INVMG (SEQ ID NO: 6), TYRMG (SEQ ID NO: 7), RYAMG (SEQ ID NO: 8), FTTYRMG (SEQ ID NO: 258), TYRMG (SEQ ID NO: 259) or FNTYAMG (SEQ ID NO: 9).

36. The method of any one of embodiments 1 to 34, wherein the VHH domain comprises a complementarity determining region 1 (CDR1) comprising the amino acid sequence of GLTFSSY (SEQ ID NO: 10), GSIFSIN (SEQ ID NO: 11), GTSVSSN (SEQ ID NO: 12), GSSVSSD (SEQ ID NO: 14), RSIGSIN (SEQ ID NO: 15), GRTFSSY (SEQ ID NO: 13), GRTFSTY (SEQ ID NO: 16), GFTFTRY (SEQ ID NO: 17), GRTFTTY (SEQ ID NO: 18), or GRTLSFNTY (SEQ ID NO: 19).

37. The method of any one of embodiments 1 to 34, wherein the VHH domain comprises a complementarity determining region 1 (CDR1) comprising the amino acid sequence of (i) GLTFSSYR (SEQ ID NO: 20), GSIFSINV (SEQ ID NO: 21), GTSVSSNA (SEQ ID NO: 22), GRTFSSYA (SEQ ID NO: 23), GSSVSSDA (SEQ ID NO: 24), RSIGSINV (SEQ ID NO: 25), GRTFSTYR (SEQ ID NO: 26), GFTFTRYA (SEQ ID NO: 27), GRTFTTYR (SEQ ID NO: 28), or GRTLSFNTYA (SEQ ID NO: 29); (ii) GLTFSSYRMG (SEQ ID NO: 154), GSIFSINVMG (SEQ ID NO: 155), GTSVSSNAMG (SEQ ID NO: 156), GRTFSSYAMG (SEQ ID NO: 157), GSSVSSDAMG (SEQ ID NO: 158), RSIGSINVMG (SEQ ID NO: 159), GRTFSTYRMG (SEQ ID NO: 160), GFTFTRYAMG (SEQ ID NO: 161), GRTFTTYRMG (SEQ ID NO: 162), or GRTLSFNTYAMG (SEQ ID NO: 163); (iii) SSYRMG (SEQ ID NO: 164), SINVMG (SEQ ID NO: 165), SSNAMG (SEQ ID NO: 166), SSYAMG (SEQ ID NO: 167), SSDAMG (SEQ ID NO: 168), SINVMG (SEQ ID NO: 169), STYRMG (SEQ ID NO: 170), TRYAMG (SEQ ID NO: 171), TTYRMG (SEQ ID NO: 172), or SFNTYAMG (SEQ ID NO: 173); or (iv) GLTFSSYRMG (SEQ ID NO: 174), GSIFSINVMG (SEQ ID NO: 175), GTSVSSNAMG (SEQ ID NO: 176), GRTFSSYAMG (SEQ ID NO: 177), GSSVSSDAMG (SEQ ID NO: 178), RSIGSINVMG (SEQ ID NO: 179), GRTFSTYRMG (SEQ ID NO: 180), GFTFTRYAMG (SEQ ID NO: 181), GRTFTTYRMG (SEQ ID NO: 182), or GRTLSFNTYAMG (SEQ ID NO: 183).

38. The method of any one of embodiments 1 to 37, wherein the VHH domain comprises a complementarity determining region 2 (CDR2) comprising the amino acid sequence of AIDWNGRGTYYRYYADSVKG (SEQ ID NO: 30), RINGGGITHYAESVKG (SEQ ID NO: 31), FIDRIATTTIATSVKG (SEQ ID NO: 32), AITWNGGTTYYADSVKG (SEQ ID NO: 33), FISGGGTTTYADSVKG (SEQ ID NO: 34), RITGGGSTHYAESVKG (SEQ ID NO: 35), AISWSGGSTTYADPVKG (SEQ ID NO: 36), AISWSGSSAGYGDSVKG (SEQ ID NO: 37), AIRWSGGRTLYADSVKG (SEQ ID NO: 38), or SITWNGGSTSYADSVKG (SEQ ID NO: 39).

39. The method of any one of embodiments 1 to 37, wherein the VHH domain comprises a complementarity determining region 2 (CDR2) comprising the amino acid sequence of DWNGRGTYY (SEQ ID NO: 40), WNGRGTY (SEQ ID NO: 260), NGGGI (SEQ ID NO: 41), GGG (SEQ ID NO: 261), DRIAT (SEQ ID NO: 42), RIA (SEQ ID NO: 262), TWNGGT (SEQ ID NO: 43), WNGG (SEQ ID NO: 263), SGGGT (SEQ ID NO: 44), GGG (SEQ ID NO: 264), TGGGS (SEQ ID NO: 45), GGG (SEQ ID NO: 265), SWSGGS (SEQ ID NO: 46), WSGG (SEQ ID NO: 266), SWSGSS (SEQ ID NO: 47), WSGS (SEQ ID NO: 267), RWSGGR (SEQ ID NO: 48), WSGG (SEQ ID NO: 268), TWNGGS (SEQ ID NO: 49) or WNGG (SEQ ID NO: 269).

40. The method of any one of embodiments 1 to 37, wherein the VHH domain comprises a complementarity determining region 2 (CDR2) comprising the amino acid sequence of (i) IDWNGRGTYY (SEQ ID NO: 50), IDWNGRGTYYR (SEQ ID NO: 270), INGGGIT (SEQ ID NO: 51), IDRIATT (SEQ ID NO: 52), ITWNGGTT (SEQ ID NO: 53), ISGGGTT (SEQ ID NO: 54), ITGGGST (SEQ ID NO: 55), ISWSGGST (SEQ ID NO: 56), ISWSGSSA (SEQ ID NO: 57), IRWSGGRT (SEQ ID NO: 58), or ITWNGGST (SEQ ID NO: 59); (ii) AIDWNGRGTYYRYYADSVKG (SEQ ID NO: 184), RINGGGITHYAESVKG (SEQ ID NO: 185), FIDRIATTTIATSVKG (SEQ ID NO: 186), AITWNGGTTYYADSVKG (SEQ ID NO: 187), FISGGGTTTYADSVKG (SEQ ID NO: 188), RITGGGSTHYAESVKG (SEQ ID NO: 189), AISWSGGSTTYADPVKG (SEQ ID NO: 190), AISWSGSSAGYGDSVKG (SEQ ID NO: 191), AIRWSGGRTLYADSVKG (SEQ ID NO: 192), or SITWNGGSTSYADSVKG (SEQ ID NO: 193); (iii) FVAAIDWNGRGTYYRY (SEQ ID NO: 194), LVARINGGGITH (SEQ ID NO: 195), WVGFIDRIATTT (SEQ ID NO: 196), FVAAITWNGGTTY (SEQ ID NO: 197), WVAFISGGGTTT (SEQ ID NO: 198), LVARITGGGSTH (SEQ ID NO: 199), FVAAISWSGGSTT (SEQ ID NO: 200), FVAAISWSGSSAG (SEQ ID NO: 201), FVAAIRWSGGRTL (SEQ ID NO: 202), or FVASITWNGGSTS (SEQ ID NO: 203); or (iv) AIDWNGRGTYYRY (SEQ ID NO: 204), RINGGGITH (SEQ ID NO: 205), FIDRIATTT (SEQ ID NO: 206), AITWNGGTTY (SEQ ID NO: 207), FISGGGTTT (SEQ ID NO: 208), RITGGGSTH (SEQ ID NO: 209), AISWSGGSTT (SEQ ID NO: 210), AISWSGSSAG (SEQ ID NO: 211), AIRWSGGRTL (SEQ ID NO: 212), or SITWNGGSTS (SEQ ID NO: 213).

41. The method of any one of embodiments 1 to 40, wherein the VHH domain comprises a complementarity determining region 3 (CDR3) comprising the amino acid sequence of GSIDLNWYGGMDY (SEQ ID NO: 60), TTVLTDPRVLNEYAT (SEQ ID NO: 61), DVFGSSGYVETY (SEQ ID NO: 62), PLTAR (SEQ ID NO: 63), DPFNQGY (SEQ ID NO: 64), PLTSR (SEQ ID NO: 65), MVNPIITAWGTIGVREIPDYDY (SEQ ID NO: 66), DQRGY (SEQ ID NO: 67), QRGY (SEQ ID NO: 271), DPFNQGY (SEQ ID NO: 68), DLAEYSGTYSSPADSPAGYDY (SEQ ID NO: 69), or ARYYVSGTYFPANY (SEQ ID NO: 70).

42. The method of any one of embodiments 1 to 40, wherein the VHH domain comprises a complementarity determining region 3 (CDR3) comprising the amino acid sequence of GSIDLNWYGGMDY (SEQ ID NO: 71), SIDLNWYGGMD (SEQ ID NO: 272), TTVLTDPRVLNEYAT (SEQ ID NO: 72), TVLTDPRVLNEYA (SEQ ID NO: 273), DVFGSSGYVETY (SEQ ID NO: 73), VFGSSGYVET (SEQ ID NO: 274), PLTAR (SEQ ID NO: 74), LTA (SEQ ID NO: 275), DPFNQGY (SEQ ID NO: 75), PFNQG (SEQ ID NO: 276), PLTSR (SEQ ID NO: 76), LTS (SEQ ID NO: 277), MVNPIITAWGTIGVREIPDYDY (SEQ ID NO: 77), VNPIITAWGTIGVREIPDYD (SEQ ID NO: 278), DQRGY (SEQ ID NO: 78), RG (SEQ ID NO: 279), DPFNQGY (SEQ ID NO: 79), PFNQG (SEQ ID NO: 280), DLAEYSGTYSSPADSPAGYDY (SEQ ID NO: 80), LAEYSGTYSSPADSPAGYD (SEQ ID NO: 281), ARYYVSGTYFPANY (SEQ ID NO: 81), or RYYVSGTYFPAN (SEQ ID NO: 282).

43. The method of any one of embodiments 1 to 40, wherein the VHH domain comprises a complementarity determining region 3 (CDR3) comprising the amino acid sequence of (i) CAAGSIDLNWYGGMDY (SEQ ID NO: 82), AAGSIDLNWYGGMDY (SEQ ID NO: 283), CAATTVLTDPRVLNEYAT (SEQ ID NO: 83), AATTVLTDPRVLNEYAT (SEQ ID NO: 284), KADVFGSSGYVETY (SEQ ID NO: 84), NHPLTAR (SEQ ID NO: 85), AADPFNQGY (SEQ ID NO: 86), NHPLTSR (SEQ ID NO: 87), ASMVNPIITAWGTIGVREIPDYDY (SEQ ID NO: 88), NDQRGY (SEQ ID NO: 89), AADPFNQGY (SEQ ID NO: 90), AADLAEYSGTYSSPADSPAGYDY (SEQ ID NO: 91), or AAARYYVSGTYFPANY (SEQ ID NO: 92); (ii) GSIDLNWYGGMDY (SEQ ID NO: 214), TTVLTDPRVLNEYAT (SEQ ID NO: 215), DVFGSSGYVETY (SEQ ID NO: 216), PLTAR (SEQ ID NO: 217), DPFNQGY (SEQ ID NO: 218), PLTSR (SEQ ID NO: 219), MVNPIITAWGTIGVREIPDYDY (SEQ ID NO: 220), QRGY (SEQ ID NO: 221), DPFNQGY (SEQ ID NO: 222), DLAEYSGTYSSPADSPAGYDY (SEQ ID NO: 223), or ARYYVSGTYFPANY (SEQ ID NO: 224); (iii) AAGSIDLNWYGGMD (SEQ ID NO: 225), AATTVLTDPRVLNEYA (SEQ ID NO: 226), KADVFGSSGYVET (SEQ ID NO: 227), NHPLTA (SEQ ID NO: 228), AADPFNQG (SEQ ID NO: 229), NHPLTS (SEQ ID NO: 230), ASMVNPIITAWGTIGVREIPDYD (SEQ ID NO: 231), NDQRG (SEQ ID NO: 232), AADPFNQG (SEQ ID NO: 233), AADLAEYSGTYSSPADSPAGYD (SEQ ID NO: 234), or AAARYYVSGTYFPAN (SEQ ID NO: 235); or (iv) GSIDLNWYGGMDY (SEQ ID NO: 236), TTVLTDPRVLNEYAT (SEQ ID NO: 237), DVFGSSGYVETY (SEQ ID NO: 238), PLTAR (SEQ ID NO: 239), DPFNQGY (SEQ ID NO: 240), PLTSR (SEQ ID NO: 241), MVNPIITAWGTIGVREIPDYDY (SEQ ID NO: 242), QRGY (SEQ ID NO: 243), DPFNQGY (SEQ ID NO: 244), DLAEYSGTYSSPADSPAGYDY (SEQ ID NO: 245), or ARYYVSGTYFPANY (SEQ ID NO: 246),

including wherein optionally the VHH domain comprises a CDR1 sequence, a CDR2 sequence, and a CDR3 sequence of the VHH domain selected from the group consisting of:
a) VHH1:
   i) the CDR1 sequence of SYRMG (SEQ ID NO: 1), the CDR2 sequence of AIDWNGRGTYYRYYADSVKG (SEQ ID NO: 30), and the CDR3 sequence of GSIDLNWYGGMDY (SEQ ID NO: 60);
   ii) the CDR1 sequence of GLTFSSY (SEQ ID NO: 10), the CDR2 sequence of DWNGRGTYY (SEQ ID NO: 40) or WNGRGTY (SEQ ID NO: 260), and the CDR3 sequence of GSIDLNWYGGMDY (SEQ ID NO: 71) or SIDLNWYGGMD (SEQ ID NO: 272);
   iii) the CDR1 sequence of GLTFSSYR (SEQ ID NO: 20), the CDR2 sequence of IDWNGRGTYY (SEQ ID NO: 50) or IDWNGRGTYYR (SEQ ID NO: 270), and the CDR3 sequence of CAAGSIDLNWYGGMDY (SEQ ID NO: 82) or AAGSIDLNWYGGMDY (SEQ ID NO: 283);
   iv) the CDR1 sequence of GLTFSSYRMG (SEQ ID NO: 154), the CDR 2 sequence of AIDWNGRGTYYRYYADSVKG (SEQ ID NO: 184), and the CDR3 sequence of GSIDLNWYGGMDY (SEQ ID NO: 214);
   v) the CDR1 sequence of SSYRMG (SEQ ID NO: 164), the CDR2 sequence of FVAAIDWNGRGTYYRY (SEQ ID NO: 194), and the CDR3 sequence of AAGSIDLNWYGGMD (SEQ ID NO: 225); or
   vi) the CDR1 sequence of GLTFSSYRMG (SEQ ID NO: 174), the CDR2 sequence of AIDWNGRGTYYRY (SEQ ID NO: 204), and the CDR3 sequence of GSIDLNWYGGMDY (SEQ ID NO: 236);
b) VHH2:
   i) the CDR1 sequence of SYRMG (SEQ ID NO: 1), the CDR2 sequence of AIDWNGRGTYYRYYADSVKG (SEQ ID NO: 30), and the CDR3 sequence of TTVLTDPRVLNEYAT (SEQ ID NO: 61);
   ii) the CDR1 sequence of GLTFSSY (SEQ ID NO: 10), the CDR2 sequence of DWNGRGTYY (SEQ ID NO: 40) or WNGRGTY (SEQ ID NO: 260), and the CDR3 sequence of TTVLTDPRVLNEYAT (SEQ ID NO: 72) or TVLTDPRVLNEYA (SEQ ID NO: 273);
   iii) the CDR1 sequence of GLTFSSYR (SEQ ID NO: 20), the CDR2 sequence of IDWNGRGTYY (SEQ ID NO: 50) or IDWNGRGTYYR (SEQ ID NO: 270), and the CDR3 sequence of CAATTVLTDPRVLNEYAT (SEQ ID NO: 83) or AATTVLTDPRVLNEYAT (SEQ ID NO: 284);
   iv) the CDR1 sequence of GLTFSSYRMG (SEQ ID NO: 154), the CDR2 sequence of AIDWNGRGTYYRYYADSVKG (SEQ ID NO: 184), and the CDR3 sequence of TTVLTDPRVLNEYAT (SEQ ID NO: 215);
   v) the CDR1 sequence of SSYRMG (SEQ ID NO: 164), the CDR2 sequence of FVAAIDWNGRGTYYRY (SEQ ID NO: 194), and the CDR3 sequence of AATTVLTDPRVLNEYA (SEQ ID NO: 226); or
   vi) the CDR1 sequence of GLTFSSYRMG (SEQ ID NO: 174), the CDR2 sequence of AIDWNGRGTYYRY (SEQ ID NO: 204), and the CDR3 sequence of TTVLTDPRVLNEYAT (SEQ ID NO: 237);
c) VHH3:
   i) the CDR1 sequence of INVMG (SEQ ID NO: 2), the CDR2 sequence of RINGGGITHYAESVKG (SEQ ID NO: 31), and the CDR3 sequence of DVFGSSGYVETY (SEQ ID NO: 62);
   ii) the CDR1 sequence of GSIFSIN (SEQ ID NO: 11), the CDR2 sequence of NGGGI (SEQ ID NO: 41) or GGG (SEQ ID NO: 261), and the CDR3 sequence of DVFGSSGYVETY (SEQ ID NO: 73) or VFGSSGYVET (SEQ ID NO: 274);
   iii) the CDR1 sequence of GSIFSINV (SEQ ID NO: 21), the CDR2 sequence of INGGGIT (SEQ ID NO: 51), and the CDR3 sequence of KADVFGSSGYVETY (SEQ ID NO: 84);
   iv) the CDR1 sequence of GSIFSINVMG (SEQ ID NO: 155), the CDR2 sequence of RINGGGITHYAESVKG (SEQ ID NO: 185), and the CDR3 sequence of DVFGSSGYVETY (SEQ ID NO: 216);
   v) the CDR1 sequence of SINVMG (SEQ ID NO: 165), the CDR2 sequence of LVARINGGGITH (SEQ ID NO: 195), and the CDR3 sequence of KADVFGSSGYVET (SEQ ID NO: 227); or
   vi) the CDR1 sequence of GSIFSINVMG (SEQ ID NO: 175), the CDR2 sequence of RINGGGITH (SEQ ID NO: 205), and the CDR3 sequence of DVFGSSGYVETY (SEQ ID NO: 238);
d) VHH4:
   i) the CDR1 sequence of SNAMG (SEQ ID NO: 3), the CDR2 sequence of FIDRIATTTIATSVKG (SEQ ID NO: 32), and the CDR3 sequence of PLTAR (SEQ ID NO: 63);
   ii) the CDR1 sequence of GTSVSSN (SEQ ID NO: 12), the CDR2 sequence of DRIAT (SEQ ID NO: 42) or RIA (SEQ ID NO: 262), and the CDR3 sequence of PLTAR (SEQ ID NO: 74) or LTA (SEQ ID NO: 275);
   iii) the CDR1 sequence of GTSVSSNA (SEQ ID NO: 22), the CDR2 sequence of IDRIATT (SEQ ID NO: 52), and the CDR3 sequence of NHPLTAR (SEQ ID NO: 85);
   iv) the CDR1 sequence of GTSVSSNAMG (SEQ ID NO: 156), the CDR2 sequence of FIDRIATTTIATSVKG (SEQ ID NO: 186), and the CDR3 sequence of PLTAR (SEQ ID NO: 217);
   v) the CDR1 sequence of SSNAMG (SEQ ID NO: 166), the CDR2 sequence of WVGFIDRIATTT (SEQ ID NO: 196), and the CDR3 sequence of NHPLTA (SEQ ID NO: 228); or
   vi) the CDR1 sequence of GTSVSSNAMG (SEQ ID NO: 176), the CDR2 sequence of FIDRIATTT (SEQ ID NO: 206), and the CDR3 sequence of PLTAR (SEQ ID NO: 239);
e) VHH5:
   i) the CDR1 sequence of SYAMG (SEQ ID NO: 4), the CDR2 sequence of AITWNGGTTYYADSVKG (SEQ ID NO: 33), and the CDR3 sequence of DPFNQGY (SEQ ID NO: 64);
   ii) the CDR1 sequence of GRTFSSY (SEQ ID NO: 13), the CDR2 sequence of TWNGGT (SEQ ID NO: 43) or WNGG (SEQ ID NO: 263), and the CDR3 sequence of DPFNQGY (SEQ ID NO: 75) or PFNQG (SEQ ID NO: 276);
   iii) the CDR1 sequence of GRTFSSYA (SEQ ID NO: 23), the CDR2 sequence of ITWNGGTT (SEQ ID NO: 53), and the CDR3 sequence of AADPFNQGY (SEQ ID NO: 86);
   iv) the CDR1 sequence of GRTFSSYAMG (SEQ ID NO: 157), the CDR2 sequence of AITWNGGTTYYADSVKG (SEQ ID NO: 187), and the CDR3 sequence of DPFNQGY (SEQ ID NO: 218);
   v) the CDR1 sequence of SSYAMG (SEQ ID NO: 167), the CDR2 sequence of FVAAITWNGGTTY (SEQ ID NO: 197), and the CDR3 sequence of AADPFNQG (SEQ ID NO: 229); or
   vi) the CDR1 sequence of GRTFSSYAMG (SEQ ID NO: 177), the CDR2 sequence of AITWNGGTTY (SEQ ID NO: 207), and the CDR3 sequence of DPFNQGY (SEQ ID NO: 240);
f) VHH6:
   i) the CDR1 sequence of SDAMG (SEQ ID NO: 5), the CDR2 sequence of FISGGGTTTYADSVKG (SEQ ID NO: 34), and the CDR3 sequence of PLTSR (SEQ ID NO: 65);
   ii) the CDR1 sequence of GSSVSSD (SEQ ID NO: 14), the CDR2 sequence of SGGGT (SEQ ID NO: 44) or GGG (SEQ ID NO: 264), and the CDR3 sequence of PLTSR (SEQ ID NO: 76) or LTS (SEQ ID NO: 277);
   iii) the CDR1 sequence of GSSVSSDA (SEQ ID NO: 24), the CDR2 sequence of ISGGGTT (SEQ ID NO: 54), and the CDR3 sequence of NHPLTSR (SEQ ID NO: 87);
   iv) the CDR1 sequence of GSSVSSDAMG (SEQ ID NO: 158), the CDR2 sequence of FISGGGTTTYADSVKG (SEQ ID NO: 188), and the CDR3 sequence of PLTSR (SEQ ID NO: 219);
   v) the CDR1 sequence of SSDAMG (SEQ ID NO: 168), the CDR2 sequence of WVAFISGGGTTT (SEQ ID NO: 198), and the CDR3 sequence of NHPLTS (SEQ ID NO: 230); or
   vi) the CDR1 sequence of GSSVSSDAMG (SEQ ID NO: 178), the CDR2 sequence of FISGGGTTT (SEQ ID NO: 208), and the CDR3 sequence of PLTSR (SEQ ID NO: 241);
g) VHH7:
   i) the CDR1 sequence of INVMG (SEQ ID NO: 6), the CDR2 sequence of RITGGGSTHYAESVKG (SEQ ID NO: 35), and the CDR3 sequence of MVNPIITAWGTIGVREIPDYDY (SEQ ID NO: 66);
   ii) the CDR1 sequence of RSIGSIN (SEQ ID NO: 15), the CDR2 sequence of TGGGS (SEQ ID NO: 45) or GGG (SEQ ID NO: 265), and the CDR3 sequence of MVNPIITAWGTIGVREIPDYDY (SEQ ID NO: 77) or VNPIITAWGTIGVREIPDYD (SEQ ID NO: 278);
   iii) the CDR1 sequence of RSIGSINV (SEQ ID NO: 25), the CDR2 sequence of ITGGGST (SEQ ID NO: 55), and the CDR3 sequence of ASMVNPIITAWGTIGVREIPDYDY (SEQ ID NO: 88);
   iv) the CDR1 sequence of RSIGSINVMG (SEQ ID NO: 159), the CDR2 sequence of RITGGGSTHYAESVKG (SEQ ID NO: 189), and the CDR3 sequence of MVNPIITAWGTIGVREIPDYDY (SEQ ID NO: 220);
   v) the CDR1 sequence of SINVMG (SEQ ID NO: 169), the CDR2 sequence of LVARITGGGSTH (SEQ ID NO: 199), and the CDR3 sequence of ASMVNPIITAWGTIGVREIPDYD (SEQ ID NO: 231); or
   vi) the CDR1 sequence of RSIGSINVMG (SEQ ID NO: 179), the CDR2 sequence of RITGGGSTH (SEQ ID NO: 209), and the CDR3 sequence of MVNPIITAWGTIGVREIPDYDY (SEQ ID NO: 242);
h) VHH9:
   i) the CDR1 sequence of TYRMG (SEQ ID NO: 7), the CDR2 sequence of AISWSGGSTTYADPVKG (SEQ ID NO: 36), and the CDR3 sequence of DQRGY (SEQ ID NO: 67) or QRGY (SEQ ID NO: 271);
   ii) the CDR1 sequence of GRTFSTY (SEQ ID NO: 16), the CDR2 sequence of SWSGGS (SEQ ID NO: 46) or WSGG (SEQ ID NO: 266), and the CDR3 sequence of DQRGY (SEQ ID NO: 78) or RG (SEQ ID NO: 279);
   iii) the CDR1 sequence of GRTFSTYR (SEQ ID NO: 26), the CDR2 sequence of ISWSGGST (SEQ ID NO: 56), and the CDR3 sequence of NDQRGY (SEQ ID NO: 89);
   iv) the CDR1 sequence of GRTFSTYRMG (SEQ ID NO: 160), the CDR2 sequence of AISWSGGSTTYADPVKG (SEQ ID NO: 190), and the CDR3 sequence of QRGY (SEQ ID NO: 221);
   v) the CDR1 sequence of STYRMG (SEQ ID NO: 170), the CDR2 sequence of FVAAISWSGGSTT (SEQ ID NO: 200), and the CDR3 sequence of NDQRG (SEQ ID NO: 232); or
   vi) the CDR1 sequence of GRTFSTYRMG (SEQ ID NO: 180), the CDR2 sequence of AISWSGGSTT (SEQ ID NO: 210), and the CDR3 sequence of QRGY (SEQ ID NO: 243);
i) VHH10:
   i) the CDR1 sequence of RYAMG (SEQ ID NO: 8), the CDR2 sequence of AISWSGSSAGYGDSVKG (SEQ ID NO: 37), and the CDR3 sequence of DPFNQGY (SEQ ID NO: 68);
   ii) the CDR1 sequence of GFTFTRY (SEQ ID NO: 17), the CDR2 sequence of SWSGSS (SEQ ID NO: 47) or WSGS (SEQ ID NO: 267), and the CDR3 sequence of DPFNQGY (SEQ ID NO: 79) or PFNQG (SEQ ID NO: 280);
   iii) the CDR1 sequence of GFTFTRYA (SEQ ID NO: 27), the CDR2 sequence of ISWSGSSA (SEQ ID NO: 57), and the CDR3 sequence of AADPFNQGY (SEQ ID NO: 90);
   iv) the CDR1 sequence of GFTFTRYAMG (SEQ ID NO: 161), the CDR2 sequence of AISWSGSSAGYGDSVKG (SEQ ID NO: 191), and the CDR3 sequence of DPFNQGY (SEQ ID NO: 222);
   v) the CDR1 sequence of TRYAMG (SEQ ID NO: 171), the CDR2 sequence of FVAAISWSGSSAG (SEQ ID NO: 201), and the CDR3 sequence of AADPFNQG (SEQ ID NO: 233); or
   vi) the CDR1 sequence of GFTFTRYAMG (SEQ ID NO: 181), the CDR2 sequence of AISWSGSSAG (SEQ ID NO: 211), and the CDR3 sequence of DPFNQGY (SEQ ID NO: 244);
j) VHH11:
   i) the CDR1 sequence of FTTYRMG (SEQ ID NO: 258) or TYRMG (SEQ ID NO: 259), the CDR2 sequence of AIRWSGGRTLYADSVKG (SEQ ID NO: 38), and the CDR3 sequence of DLAEYSGTYSSPADSPAGYDY (SEQ ID NO: 69);
   ii) the CDR1 sequence of GRTFTTY (SEQ ID NO: 18), the CDR2 sequence of RWSGGR (SEQ ID NO: 48) or WSGG (SEQ ID NO: 268), and the CDR3 sequence of DLAEYSGTYSSPADSPAGYDY (SEQ ID NO: 80) or LAEYSGTYSSPADSPAGYD (SEQ ID NO: 281);
   iii) the CDR1 sequence of GRTFTTYR (SEQ ID NO: 28), the CDR2 sequence of IRWSGGRT (SEQ ID NO: 58), and the CDR3 sequence of AADLAEYSGTYSSPADSPAGYDY (SEQ ID NO: 91);
   iv) the CDR1 sequence of GRTFTTYRMG (SEQ ID NO: 162), the CDR2 sequence of AIRWSGGRTLYADSVKG (SEQ ID NO: 192), and the CDR3 sequence of DLAEYSGTYSSPADSPAGYDY (SEQ ID NO: 223);
   v) the CDR1 sequence of TTYRMG (SEQ ID NO: 172), the CDR2 sequence of FVAAIRWSGGRTL (SEQ ID NO: 202), and the CDR3 sequence of AADLAEYSGTYSSPADSPAGYD (SEQ ID NO: 234); or
   vi) the CDR1 sequence of GRTFTTYRMG (SEQ ID NO: 182), the CDR2 sequence of AIRWSGGRTL (SEQ ID NO: 212), and the CDR3 sequence of DLAEYSGTYSSPADSPAGYDY (SEQ ID NO: 245); and
k) VHH12:
   i) the CDR1 sequence of FNTYAMG (SEQ ID NO: 9), the CDR2 sequence of SITWNGGSTSYADSVKG (SEQ ID NO: 39), and the CDR3 sequence of ARYYVSGTYFPANY (SEQ ID NO: 70);
   ii) the CDR1 sequence of GRTLSFNTY (SEQ ID NO: 19), the CDR2 sequence of TWNGGS (SEQ ID NO: 49) or WNGG (SEQ ID NO: 269), and the CDR3 sequence of ARYYVSGTYFPANY (SEQ ID NO: 81) or RYYVSGTYFPAN (SEQ ID NO: 282);
   iii) the CDR1 sequence of GRTLSFNTYA (SEQ ID NO: 29), the CDR2 sequence of ITWNGGST (SEQ ID NO: 59), and the CDR3 sequence of AAARYYVSGTYFPANY (SEQ ID NO: 92);
   iv) the CDR1 sequence of GRTLSFNTYAMG (SEQ ID NO: 163), the CDR2 sequence of SITWNGGSTSYADSVKG (SEQ ID NO: 193), and the CDR3 sequence of ARYYVSGTYFPANY (SEQ ID NO: 224);
   v) the CDR1 sequence of SFNTYAMG (SEQ ID NO: 173), the CDR2 sequence of FVASITWNGGSTS (SEQ ID NO: 203), and the CDR3 sequence of AAARYYVSGTYFPAN (SEQ ID NO: 235); or
   vi) the CDR1 sequence of GRTLSFNTYAMG (SEQ ID NO: 183), the CDR2 sequence of SITWNGGSTS (SEQ ID NO: 213), and the CDR3 sequence of ARYYVSGTYFPANY (SEQ ID NO: 246).

44. The method of any one of embodiments 1 to 34, wherein the VHH domain is comprised of a germline sequence of

(SEQ ID NO: 285)
QVQLVESGGGLVQAGGSLKLACAAPGLTFSSYRMGWFRQAPGQEREFVA
AIDWNGRGTYYRYYADSVKGRSTISRDNAKNTMYLQMNSLKPEDTAVYY
CA,
(SEQ ID NO: 286)
EVQVVESGGGLVQAGGSLKLACAAPGLTFSSYRMGWFRQAPGQEREFVA
AIDWNGRGTYYRYYADSVKGRSTISRDNAKNTVYLQMNSLKPEDTAVYY,
CA
(SEQ ID NO: 287)
QLQLVESGGGLVQPGGSLRLSCAASGSIFSINVMGWYRQAPGKQRELVA
RINGGGITHYAESVKGRFTISRDNAKNTVYLQMNSLKPEDTAAYYCKA,
(SEQ ID NO: 288)
EVQVVESGGGLVQAGGSLRLSCAVSGTSVSSNAMGWYRQAPGKQREWVG
FIDRIATTTIATSVKGRFAITRDNAKNTVYLQMSGLKPEDTAVYYCN,
(SEQ ID NO: 289)
QVQLVESGGGLVQAGGSLRLSCAASGRTFSSYAMGWFRQAPGKEREFVA
AITWNGGTTYYADSVKGRFTISRDNAKNTVYLQMNSLKPEDTAVYYCAA,
(SEQ ID NO: 290)
EVQLVESGGGLVQAGGSLRLSCAVSGSSVSSDAMGWYRQAPGNQRAWVA
FISGGGTTTYADSVKGRFTISRDNTKNTVYLHMNSLKPEDTAVYYCN,
(SEQ ID NO: 291)
EVQVVESGGGLVQAGGSLRLACVASRSIGSINVMGWYRQAPGKQRDLVA
RITGGGSTHYAESVKGRFTISRDNAKNTVYLQMNSLEPEDTAVYYC,
(SEQ ID NO: 292)
QVQLVESGGGLVQAGGSLRLSCAVSGRTFSTYRMGWFRQAPGKERSFVA
AISWSGGSTTYADPVKGRFTISRDNAKNTVYLRMNSLKPEDTAVYYCN,
(SEQ ID NO: 293)
EVQVVESGGGLVQAGGSLRLSCAASGFTFTRYAMGWFRQAPGKERSFVA
AISWSGSSAGYGDSVKGRFTISRDNAKNTLYLQMNSLKPEDTAVYYCA,
(SEQ ID NO: 294)
EVQVVESGGGLVQAGGSLRLSCAASGRTFTTYRMGWFRQAPGKEREFVA
AIRWSGGRTLYADSVKGRFTISRDNAKNTAYLQMNNLRPEDTAVYYCAA,
or
(SEQ ID NO: 295)
QVQLVETGGGLVQAGDSLRLSCAASGRTLSFNTYAMGWFRQAPGKEREFV
ASITWNGGSTSYADSVKGRFTITRDNAKNTATLRMNSLQPDDTAVYYCA
A.

45. The method of any one of embodiments 1 to 44, wherein the VHH domain is comprised of a J segment, wherein the J segment comprises the sequence of

(SEQ ID NO: 296)
DYWGQGTQVTVSS,
(SEQ ID NO: 297)
EYATWGQGTQVTVSS,
(SEQ ID NO: 298)
YWGQGTQVTVSS,
(SEQ ID NO: 299)
WGQGTQVTVSS,
(SEQ ID NO: 300)
DYDYWGQGTQVTVSS,
(SEQ ID NO: 301)
WGQGTLVTVSS,
(SEQ ID NO: 302)
YDYWGQGTQVTVSS,
or
(SEQ ID NO: 303)
NYWGQGTQVTVSS.

46. The method of any one of embodiments 1 to 34, wherein the VHH domain is comprised of a sequence of

(SEQ ID NO: 93)
QVQLVESGGGLVQAGGSLKLACAAPGLTFSSYRMGWFRQAPGQEREFVA
AIDWNGRGTYYRYYADSVKGRSTISRDNAKNTMYLQMNSLKPEDTAVYY
CAAGSIDLNWYGGMDYWGQGTQVTVSS,
(SEQ ID NO: 94)
EVQVVESGGGLVQAGGSLKLACAAPGLTFSSYRMGWFRQAPGQEREFVA
AIDWNGRGTYYRYYADSVKGRSTISRDNAKNTVYLQMNSLKPEDTAVYY
CAATTVLTDPRVLNEYATWGQGTQVTVSS,
(SEQ ID NO: 95)
QLQLVESGGGLVQPGGSLRLSCAASGSIFSINVMGWYRQAPGKQRELVA
RINGGGITHYAESVKGRFTISRDNAKNTVYLQMNSLKPEDTAAYYCKAD
VFGSSGYVETYWGQGTQVTVSS,
(SEQ ID NO: 96)
EVQVVESGGGLVQAGGSLRLSCAVSGTSVSSNAMGWYRQAPGKQREWVG
FIDRIATTTIATSVKGRFAITRDNAKNTVYLQMSGLKPEDTAVYYCNHP
LTARWGQGTQVTVSS,
(SEQ ID NO: 97)
QVQLVESGGGLVQAGGSLRLSCAASGRTFSSYAMGWFRQAPGKEREFVA
AITWNGGTTYYADSVKGRFTISRDNAKNTVYLQMNSLKPEDTAVYYCAA
DPFNQGYWGQGTQVTVSS,
(SEQ ID NO: 98)
EVQLVESGGGLVQAGGSLRLSCAVSGSSVSSDAMGWYRQAPGNQRAWVA
FISGGGTTTYADSVKGRFTISRDNTKNTVYLHMNSLKPEDTAVYYCNHP
LTSRWGQGTQVTVSS,
(SEQ ID NO: 99)
EVQVVESGGGLVQAGGSLRLACVASRSIGSINVMGWYRQAPGKQRDLVA
RITGGGSTHYAESVKGRFTISRDNAKNTVYLQMNSLEPEDTAVYYCASM
VNPIITAWGTIGVREIPDYDYWGQGTQVTVSS,
(SEQ ID NO: 100)
QVQLVESGGGLVQAGGSLRLSCAVSGRTFSTYRMGWFRQAPGKERSFVA
AISWSGGSTTYADPVKGRFTISRDNAKNTVYLRMNSLKPEDTAVYYCND
QRGYWGQGTLVTVSS,
(SEQ ID NO: 101)
EVQVVESGGGLVQAGGSLRLSCAASGFTFTRYAMGWFRQAPGKERSFVA
AISWSGSSAGYGDSVKGRFTISRDNAKNTLYLQMNSLKPEDTAVYYCAA
DPFNQGYWGQGTQVTVSS,
(SEQ ID NO: 102)
EVQVVESGGGLVQAGGSLRLSCAASGRTFTTYRMGWFRQAPGKEREFVA
AIRWSGGRTLYADSVKGRFTISRDNAKNTAYLQMNNLRPEDTAVYYCAA
DLAEYSGTYSSPADSPAGYDYWGQGTQVTVSS,
or
(SEQ ID NO: 103)
QVQLVETGGGLVQAGDSLRLSCAASGRTLSFNTYAMGWFRQAPGKEREF
VASITWNGGSTSYADSVKGRFTITRDNAKNTATLRMNSLQPDDTAVYYC
AAARYYVSGTYFPANYWGQGTQVTVSS.

47. The method of any one of embodiments 1 to 34, wherein the VHH domain comprises a complementarity determining region 1 (CDR1) comprising the amino acid sequence of SYRMG (SEQ ID NO: 1), SNAMG (SEQ ID NO: 3), SYAMG (SEQ ID NO: 4), SDAMG (SEQ ID NO: 5), INVMG (SEQ ID NO: 6), TYRMG (SEQ ID NO: 7), RYAMG (SEQ ID NO: 8), FTTYRMG (SEQ ID NO: 258), TYRMG (SEQ ID NO: 259), or FNTYAMG (SEQ ID NO: 9).

48. The method of any one of embodiments 1 to 34, wherein the VHH domain comprises a complementarity determining region 1 (CDR1) comprising the amino acid sequence of GLTFSSY (SEQ ID NO: 10), GTSVSSN (SEQ ID NO: 12), GSSVSSD (SEQ ID NO: 14), RSIGSIN (SEQ ID NO: 15), GRTFSSY (SEQ ID NO: 13), GRTFSTY (SEQ ID NO: 16), GFTFTRY (SEQ ID NO: 17), GRTFTTY (SEQ ID NO: 18), or GRTLSFNTY (SEQ ID NO: 19).

49. The method of any one of embodiments 1 to 34, wherein the VHH domain comprises a complementarity determining region 1 (CDR1) comprising the amino acid sequence of (i) GLTFSSYR (SEQ ID NO: 20), GTSVSSNA (SEQ ID NO: 22), GRTFSSYA (SEQ ID NO: 23), GSSVSSDA (SEQ ID NO: 24), RSIGSINV (SEQ ID NO: 25), GRTFSTYR (SEQ ID NO: 26), GFTFTRYA (SEQ ID NO: 27), GRTFTTYR (SEQ ID NO: 28), or GRTLSFNTYA (SEQ ID NO: 29); (ii) GLTFSSYRMG (SEQ ID NO: 154), GTSVSSNAMG (SEQ ID NO: 156), GRTFSSYAMG (SEQ ID NO: 157), GSSVSSDAMG (SEQ ID NO: 158), RSIGSINVMG (SEQ ID NO: 159), GRTFSTYRMG (SEQ ID NO: 160), GFTFTRYAMG (SEQ ID NO: 161), GRTFTTYRMG (SEQ ID NO: 162), or GRTLSFNTYAMG (SEQ ID NO: 163); (iii) SSYRMG (SEQ ID NO: 164), SSNAMG (SEQ ID NO: 166), SSYAMG (SEQ ID NO: 167), SSDAMG (SEQ ID NO: 168), SINVMG (SEQ ID NO: 169), STYRMG (SEQ ID NO: 170), TRYAMG (SEQ ID NO: 171), TTYRMG (SEQ ID NO: 172), or SFNTYAMG (SEQ ID NO: 173); or (iv) GLTFSSYRMG (SEQ ID NO: 174), GTSVSSNAMG (SEQ ID NO: 176), GRTFSSYAMG (SEQ ID NO: 177), GSSVSSDAMG (SEQ ID NO: 178), RSIGSINVMG (SEQ ID NO: 179), GRTFSTYRMG (SEQ ID NO: 180), GFTFTRYAMG (SEQ ID NO: 181), GRTFTTYRMG (SEQ ID NO: 182), or GRTLSFNTYAMG (SEQ ID NO: 183).

50. The method of any one of embodiments 1 to 34 and 47 to 49, wherein the VHH domain comprises a complementarity determining region 2 (CDR2) comprising the amino acid sequence of AIDWNGRGTYYRYYADSVKG (SEQ ID NO: 30), FIDRIATTTIATSVKG (SEQ ID NO: 32), AITWNGGTTYYADSVKG (SEQ ID NO: 33), FISGGGTTTYADSVKG (SEQ ID NO: 34), RITGGGSTHYAESVKG (SEQ ID NO: 35), AISWSGGSTTYADPVKG (SEQ ID NO: 36), AISWSGSSAGYGDSVKG (SEQ ID NO: 37), AIRWSGGRTLYADSVKG (SEQ ID NO: 38), or SITWNGGSTSYADSVKG (SEQ ID NO: 39).

51. The method of any one of embodiments 1 to 34 and 47 to 49, wherein the VHH domain comprises a complementarity determining region 2 (CDR2) comprising the amino acid sequence of DWNGRGTYY (SEQ ID NO: 40), WNGRGTY (SEQ ID NO: 260), DRIAT (SEQ ID NO: 42), RIA (SEQ ID NO: 262), TWNGGT (SEQ ID NO: 43), WNGG (SEQ ID NO: 263), SGGGT (SEQ ID NO: 44), GGG (SEQ ID NO: 264), TGGGS (SEQ ID NO: 45), GGG (SEQ ID NO: 265), SWSGGS (SEQ ID NO: 46), WSGG (SEQ ID NO: 266), SWSGSS (SEQ ID NO: 47), WSGS (SEQ ID NO: 267), RWSGGR (SEQ ID NO: 48), WSGG (SEQ ID NO: 268), TWNGGS (SEQ ID NO: 49), or WNGG (SEQ ID NO: 269).

52. The method of any one of embodiments 1 to 34 and 47 to 49, wherein the VHH domain comprises a complementarity determining region 2 (CDR2) comprising the amino acid sequence of (i) IDWNGRGTYY (SEQ ID NO: 50), IDWNGRGTYYR (SEQ ID NO: 270), IDRIATT (SEQ ID NO: 52), ITWNGGTT (SEQ ID NO: 53), ISGGGTT (SEQ ID NO: 54), ITGGGST (SEQ ID NO: 55), ISWSGGST (SEQ ID NO: 56), ISWSGSSA (SEQ ID NO: 57), IRWSGGRT (SEQ ID NO: 58), or ITWNGGST (SEQ ID NO: 59); (ii) AIDWNGRGTYYRYYADSVKG (SEQ ID NO: 184), FIDRIATTTIATSVKG (SEQ ID NO: 186), AITWNGGTTYYADSVKG (SEQ ID NO: 187), FISGGGTTTYADSVKG (SEQ ID NO: 188), RITGGGSTHYAESVKG (SEQ ID NO: 189), AISWSGGSTTYADPVKG (SEQ ID NO: 190), AISWSGSSAGYGDSVKG (SEQ ID NO: 191), AIRWSGGRTLYADSVKG (SEQ ID NO: 192), or SITWNGGSTSYADSVKG (SEQ ID NO: 193); (iii) FVAAIDWNGRGTYYRY (SEQ ID NO: 194), WVGFIDRIATTT (SEQ ID NO: 196), FVAAITWNGGTTY (SEQ ID NO: 197), WVAFISGGGTTT (SEQ ID NO: 198), LVARITGGGSTH (SEQ ID NO: 199), FVAAISWSGGSTT (SEQ ID NO: 200), FVAAISWSGSSAG (SEQ ID NO: 201), FVAAIRWSGGRTL (SEQ ID NO: 202), or FVASITWNGGSTS (SEQ ID NO: 203); or (iv) AIDWNGRGTYYRY (SEQ ID NO: 204), FIDRIATTT (SEQ ID NO: 206), AITWNGGTTY (SEQ ID NO: 207), FISGGGTTT (SEQ ID NO: 208), RITGGGSTH (SEQ ID NO: 209), AISWSGGSTT (SEQ ID NO: 210), AISWSGSSAG (SEQ ID NO: 211), AIRWSGGRTL (SEQ ID NO: 212), or SITWNGGSTS (SEQ ID NO: 213).

53. The method of any one of embodiments 1 to 34 and 47 to 52, wherein the VHH domain comprises a complementarity determining region 3 (CDR3) comprising the amino acid sequence of GSIDLNWYGGMDY (SEQ ID NO: 60), TTVLTDPRVLNEYAT (SEQ ID NO: 61), PLTAR (SEQ ID NO: 63), DPFNQGY (SEQ ID NO: 64), PLTSR (SEQ ID NO: 65), MVNPIITAWGTIGVREIPDYDY (SEQ ID NO: 66), DQRGY (SEQ ID NO: 67), QRGY (SEQ ID NO: 271), DPFNQGY (SEQ ID NO: 68), DLAEYSGTYSSPADSPAGYDY (SEQ ID NO: 69), or ARYYVSGTYFPANY (SEQ ID NO: 70).

54. The method of any one of embodiments 1 to 34 and 47 to 52, wherein the VHH domain comprises a complementarity determining region 3 (CDR3) comprising the amino acid sequence of GSIDLNWYGGMDY (SEQ ID NO: 71), SIDLNWYGGMD (SEQ ID NO: 272), TTVLTDPRVLNEYAT (SEQ ID NO: 72), TVLTDPRVLNEYA (SEQ ID NO: 273), PLTAR (SEQ ID NO: 74), LTA (SEQ ID NO: 275), DPFNQGY (SEQ ID NO: 75), PFNQG (SEQ ID NO: 276), PLTSR (SEQ ID NO: 76), LTS (SEQ ID NO: 277), MVNPIITAWGTIGVREIPDYDY (SEQ ID NO: 77), VNPIITAWGTIGVREIPDYD (SEQ ID NO: 278), DQRGY (SEQ ID NO: 78), RG (SEQ ID NO: 279), DPFNQGY (SEQ ID NO: 79), PFNQG (SEQ ID NO: 280), DLAEYSGTYSSPADSPAGYDY (SEQ ID NO: 80), LAEYSGTYSSPADSPAGYD (SEQ ID NO: 281), ARYYVSGTYFPANY (SEQ ID NO: 81) or RYYVSGTYFPAN (SEQ ID NO: 282).

55. The method of any one of embodiments 1 to 34 and 47 to 52, wherein the VHH domain comprises a complementarity determining region 3 (CDR3) comprising the amino acid sequence of (i) CAAGSIDLNWYGGMDY (SEQ ID NO: 82), AAGSIDLNWYGGMDY (SEQ ID NO: 283), CAATTVLTDPRVLNEYAT (SEQ ID NO: 83), AATTVLTDPRVLNEYAT (SEQ ID NO: 284), NHPLTAR (SEQ ID NO: 85), AADPFNQGY (SEQ ID NO: 86), NHPLTSR (SEQ ID NO: 87), ASMVNPIITAWGTIGVREIPDYDY (SEQ ID NO: 88), NDQRGY (SEQ ID NO: 89), AADPFNQGY (SEQ ID NO: 90), AADLAEYSGTYSSPADSPAGYDY (SEQ ID NO: 91), or AAARYYVSGTYFPANY (SEQ ID NO: 92); (ii) GSIDLNWYGGMDY (SEQ ID NO: 214), TTVLTDPRVLNEYAT (SEQ ID NO: 215), PLTAR (SEQ ID NO: 217), DPFNQGY (SEQ ID NO: 218), PLTSR (SEQ ID NO: 219), MVNPIITAWGTIGVREIPDYDY (SEQ ID NO: 220), QRGY (SEQ ID NO: 221), DPFNQGY (SEQ ID NO: 222), DLAEYSGTYSSPADSPAGYDY (SEQ ID NO: 223), or ARYYVSGTYFPANY (SEQ ID NO: 224); (iii) AATTVLTDPRVLNEYA (SEQ ID NO: 226), NHPLTA (SEQ ID NO: 228), AADPFNQG (SEQ ID NO: 229), NHPLTS (SEQ ID NO: 230), ASMVNPIITAWGTIGVREIPDYD (SEQ ID NO: 231), NDQRG (SEQ ID NO: 232), AADPFNQG (SEQ ID NO: 233), AADLAEYSGTYSSPADSPAGYD (SEQ ID NO: 234), or AAARYYVSGTYFPAN (SEQ ID NO: 235); or (iv) GSIDLNWYGGMDY (SEQ ID NO: 236), TTVLTDPRVLNEYAT (SEQ ID NO: 237), PLTAR (SEQ ID NO: 239), DPFNQGY (SEQ ID NO: 240), PLTSR (SEQ ID NO: 241), MVNPIITAWGTIGVREIPDYDY (SEQ ID NO: 242), QRGY (SEQ ID NO: 243), DPFNQGY (SEQ ID NO: 244), DLAEYSGTYSSPADSPAGYDY (SEQ ID NO: 245), or ARYYVSGTYFPANY (SEQ ID NO: 246), including wherein optionally the VHH domain comprises a CDR1 sequence, a CDR2 sequence, and a CDR3 sequence of the VHH domain selected from the group consisting of:
a) VHH1:
   i) the CDR1 sequence of SYRMG (SEQ ID NO: 1), the CDR2 sequence of AIDWNGRGTYYRYYADSVKG (SEQ ID NO: 30), and the CDR3 sequence of GSIDLNWYGGMDY (SEQ ID NO: 60);
   ii) the CDR1 sequence of GLTFSSY (SEQ ID NO: 10), the CDR2 sequence of DWNGRGTYY (SEQ ID NO: 40) or WNGRGTY (SEQ ID NO: 260), and the CDR3 sequence of GSIDLNWYGGMDY (SEQ ID NO: 71) or SIDLNWYGGMD (SEQ ID NO: 272);
   iii) the CDR1 sequence of GLTFSSYR (SEQ ID NO: 20), the CDR2 sequence of IDWNGRGTYY (SEQ ID NO: 50) or IDWNGRGTYYR (SEQ ID NO: 270), and the CDR3 sequence of CAAGSIDLNWYGGMDY (SEQ ID NO: 82) or AAGSIDLNWYGGMDY (SEQ ID NO: 283);
   iv) the CDR1 sequence of GLTFSSYRMG (SEQ ID NO: 154), the CDR 2 sequence of AIDWNGRGTYYRYYADSVKG (SEQ ID NO: 184), and the CDR3 sequence of GSIDLNWYGGMDY (SEQ ID NO: 214);
   v) the CDR1 sequence of SSYRMG (SEQ ID NO: 164), the CDR2 sequence of FVAAIDWNGRGTYYRY (SEQ ID NO: 194), and the CDR3 sequence of AAGSIDLNWYGGMD (SEQ ID NO: 225); or
   vi) the CDR1 sequence of GLTFSSYRMG (SEQ ID NO: 174), the CDR2 sequence of AIDWNGRGTYYRY (SEQ ID NO: 204), and the CDR3 sequence of GSIDLNWYGGMDY (SEQ ID NO: 236);
b) VHH2:
   i) the CDR1 sequence of SYRMG (SEQ ID NO: 1), the CDR2 sequence of AIDWNGRGTYYRYYADSVKG (SEQ ID NO: 30), and the CDR3 sequence of TTVLTDPRVLNEYAT (SEQ ID NO: 61);
   ii) the CDR1 sequence of GLTFSSY (SEQ ID NO: 10), the CDR2 sequence of DWNGRGTYY (SEQ ID NO: 40) or WNGRGTY (SEQ ID NO: 260), and the CDR3 sequence of TTVLTDPRVLNEYAT (SEQ ID NO: 72) or TVLTDPRVLNEYA (SEQ ID NO: 273);
   iii) the CDR1 sequence of GLTFSSYR (SEQ ID NO: 20), the CDR2 sequence of IDWNGRGTYY (SEQ ID NO: 50) or IDWNGRGTYYR (SEQ ID NO: 270), and the CDR3 sequence of CAATTVLTDPRVLNEYAT (SEQ ID NO: 83) or AATTVLTDPRVLNEYAT (SEQ ID NO: 284);
   iv) the CDR1 sequence of GLTFSSYRMG (SEQ ID NO: 154), the CDR2 sequence of AIDWNGRGTYYRYYADSVKG (SEQ ID NO: 184), and the CDR3 sequence of TTVLTDPRVLNEYAT (SEQ ID NO: 215);
   v) the CDR1 sequence of SSYRMG (SEQ ID NO: 164), the CDR2 sequence of FVAAIDWNGRGTYYRY (SEQ ID NO: 194), and the CDR3 sequence of AATTVLTDPRVLNEYA (SEQ ID NO: 226); or
   vi) the CDR1 sequence of GLTFSSYRMG (SEQ ID NO: 174), the CDR2 sequence of AIDWNGRGTYYRY (SEQ ID NO: 204), and the CDR3 sequence of TTVLTDPRVLNEYAT (SEQ ID NO: 237);
c) VHH3:
   i) the CDR1 sequence of INVMG (SEQ ID NO: 2), the CDR2 sequence of RINGGGITHYAESVKG (SEQ ID NO: 31), and the CDR3 sequence of DVFGSSGYVETY (SEQ ID NO: 62);
   ii) the CDR1 sequence of GSIFSIN (SEQ ID NO: 11), the CDR2 sequence of NGGGI (SEQ ID NO: 41) or GGG (SEQ ID NO: 261), and the CDR3 sequence of DVFGSSGYVETY (SEQ ID NO: 73) or VFGSSGYVET (SEQ ID NO: 274);
   iii) the CDR1 sequence of GSIFSINV (SEQ ID NO: 21), the CDR2 sequence of INGGGIT (SEQ ID NO: 51), and the CDR3 sequence of KADVFGSSGYVETY (SEQ ID NO: 84);
   iv) the CDR1 sequence of GSIFSINVMG (SEQ ID NO: 155), the CDR2 sequence of RINGGGITHYAESVKG (SEQ ID NO: 185), and the CDR3 sequence of DVFGSSGYVETY (SEQ ID NO: 216);
   v) the CDR1 sequence of SINVMG (SEQ ID NO: 165), the CDR2 sequence of LVARINGGGITH (SEQ ID NO: 195), and the CDR3 sequence of KADVFGSSGYVET (SEQ ID NO: 227); or
   vi) the CDR1 sequence of GSIFSINVMG (SEQ ID NO: 175), the CDR2 sequence of RINGGGITH (SEQ ID NO: 205), and the CDR3 sequence of DVFGSSGYVETY (SEQ ID NO: 238);
d) VHH4:
   i) the CDR1 sequence of SNAMG (SEQ ID NO: 3), the CDR2 sequence of FIDRIATTTIATSVKG (SEQ ID NO: 32), and the CDR3 sequence of PLTAR (SEQ ID NO: 63);
   ii) the CDR1 sequence of GTSVSSN (SEQ ID NO: 12), the CDR2 sequence of DRIAT (SEQ ID NO: 42) or RIA (SEQ ID NO: 262), and the CDR3 sequence of PLTAR (SEQ ID NO: 74) or LTA (SEQ ID NO: 275);
   iii) the CDR1 sequence of GTSVSSNA (SEQ ID NO: 22), the CDR2 sequence of IDRIATT (SEQ ID NO: 52), and the CDR3 sequence of NHPLTAR (SEQ ID NO: 85);
   iv) the CDR1 sequence of GTSVSSNAMG (SEQ ID NO: 156), the CDR2 sequence of FIDRIATTTIATSVKG (SEQ ID NO: 186), and the CDR3 sequence of PLTAR (SEQ ID NO: 217);
   v) the CDR1 sequence of SSNAMG (SEQ ID NO: 166), the CDR2 sequence of WVGFIDRIATTT (SEQ ID NO: 196), and the CDR3 sequence of NHPLTA (SEQ ID NO: 228); or
   vi) the CDR1 sequence of GTSVSSNAMG (SEQ ID NO: 176), the CDR2 sequence of FIDRIATTT (SEQ ID NO: 206), and the CDR3 sequence of PLTAR (SEQ ID NO: 239);
e) VHH5:
   i) the CDR1 sequence of SYAMG (SEQ ID NO: 4), the CDR2 sequence of AITWNGGTTYYADSVKG (SEQ ID NO: 33), and the CDR3 sequence of DPFNQGY (SEQ ID NO: 64);
   ii) the CDR1 sequence of GRTFSSY (SEQ ID NO: 13), the CDR2 sequence of TWNGGT (SEQ ID NO: 43) or WNGG (SEQ ID NO: 263), and the CDR3 sequence of DPFNQGY (SEQ ID NO: 75) or PFNQG (SEQ ID NO: 276);
   iii) the CDR1 sequence of GRTFSSYA (SEQ ID NO: 23), the CDR2 sequence of ITWNGGTT (SEQ ID NO: 53), and the CDR3 sequence of AADPFNQGY (SEQ ID NO: 86);
   iv) the CDR1 sequence of GRTFSSYAMG (SEQ ID NO: 157), the CDR2 sequence of AITWNGGTTYYADSVKG (SEQ ID NO: 187), and the CDR3 sequence of DPFNQGY (SEQ ID NO: 218);
   v) the CDR1 sequence of SSYAMG (SEQ ID NO: 167), the CDR2 sequence of FVAAITWNGGTTY (SEQ ID NO: 197), and the CDR3 sequence of AADPFNQG (SEQ ID NO: 229); or
   vi) the CDR1 sequence of GRTFSSYAMG (SEQ ID NO: 177), the CDR2 sequence of AITWNGGTTY (SEQ ID NO: 207), and the CDR3 sequence of DPFNQGY (SEQ ID NO: 240);
f) VHH6:
   i) the CDR1 sequence of SDAMG (SEQ ID NO: 5), the CDR2 sequence of FISGGGTTTYADSVKG (SEQ ID NO: 34), and the CDR3 sequence of PLTSR (SEQ ID NO: 65);
   ii) the CDR1 sequence of GSSVSSD (SEQ ID NO: 14), the CDR2 sequence of SGGGT (SEQ ID NO: 44) or GGG (SEQ ID NO: 264), and the CDR3 sequence of PLTSR (SEQ ID NO: 76) or LTS (SEQ ID NO: 277);
   iii) the CDR1 sequence of GSSVSSDA (SEQ ID NO: 24), the CDR2 sequence of ISGGGTT (SEQ ID NO: 54), and the CDR3 sequence of NHPLTSR (SEQ ID NO: 87);
   iv) the CDR1 sequence of GSSVSSDAMG (SEQ ID NO: 158), the CDR2 sequence of FISGGGTTTYADSVKG (SEQ ID NO: 188), and the CDR3 sequence of PLTSR (SEQ ID NO: 219);
   v) the CDR1 sequence of SSDAMG (SEQ ID NO: 168), the CDR2 sequence of WVAFISGGGTTT (SEQ ID NO: 198), and the CDR3 sequence of NHPLTS (SEQ ID NO: 230); or
   vi) the CDR1 sequence of GSSVSSDAMG (SEQ ID NO: 178), the CDR2 sequence of FISGGGTTT (SEQ ID NO: 208), and the CDR3 sequence of PLTSR (SEQ ID NO: 241);
g) VHH7:
   i) the CDR1 sequence of INVMG (SEQ ID NO: 6), the CDR2 sequence of RITGGGSTHYAESVKG (SEQ ID NO: 35), and the CDR3 sequence of MVNPIITAWGTIGVREIPDYDY (SEQ ID NO: 66);
   ii) the CDR1 sequence of RSIGSIN (SEQ ID NO: 15), the CDR2 sequence of TGGGS (SEQ ID NO: 45) or GGG (SEQ ID NO: 265), and the CDR3 sequence of MVNPIITAWGTIGVREIPDYDY (SEQ ID NO: 77) or VNPIITAWGTIGVREIPDYD (SEQ ID NO: 278);
   iii) the CDR1 sequence of RSIGSINV (SEQ ID NO: 25), the CDR2 sequence of ITGGGST (SEQ ID NO: 55), and the CDR3 sequence of ASMVNPIITAWGTIGVREIPDYDY (SEQ ID NO: 88);
   iv) the CDR1 sequence of RSIGSINVMG (SEQ ID NO: 159), the CDR2 sequence of RITGGGSTHYAESVKG (SEQ ID NO: 189), and the CDR3 sequence of MVNPIITAWGTIGVREIPDYDY (SEQ ID NO: 220);
   v) the CDR1 sequence of SINVMG (SEQ ID NO: 169), the CDR2 sequence of LVARITGGGSTH (SEQ ID NO: 199), and the CDR3 sequence of ASMVNPIITAWGTIGVREIPDYD (SEQ ID NO: 231); or
   vi) the CDR1 sequence of RSIGSINVMG (SEQ ID NO: 179), the CDR2 sequence of RITGGGSTH (SEQ ID NO: 209), and the CDR3 sequence of MVNPIITAWGTIGVREIPDYDY (SEQ ID NO: 242);
h) VHH9:
   i) the CDR1 sequence of TYRMG (SEQ ID NO: 7), the CDR2 sequence of AISWSGGSTTYADPVKG (SEQ ID NO: 36), and the CDR3 sequence of DQRGY (SEQ ID NO: 67) or QRGY (SEQ ID NO: 271);
   ii) the CDR1 sequence of GRTFSTY (SEQ ID NO: 16), the CDR2 sequence of SWSGGS (SEQ ID NO: 46) or WSGG (SEQ ID NO: 266), and the CDR3 sequence of DQRGY (SEQ ID NO: 78) or RG (SEQ ID NO: 279);
   iii) the CDR1 sequence of GRTFSTYR (SEQ ID NO: 26), the CDR2 sequence of ISWSGGST (SEQ ID NO: 56), and the CDR3 sequence of NDQRGY (SEQ ID NO: 89);
   iv) the CDR1 sequence of GRTFSTYRMG (SEQ ID NO: 160), the CDR2 sequence of AISWSGGSTTYADPVKG (SEQ ID NO: 190), and the CDR3 sequence of QRGY (SEQ ID NO: 221);
   v) the CDR1 sequence of STYRMG (SEQ ID NO: 170), the CDR2 sequence of FVAAISWSGGSTT (SEQ ID NO: 200), and the CDR3 sequence of NDQRG (SEQ ID NO: 232); or
   vi) the CDR1 sequence of GRTFSTYRMG (SEQ ID NO: 180), the CDR2 sequence of AISWSGGSTT (SEQ ID NO: 210), and the CDR3 sequence of QRGY (SEQ ID NO: 243);
i) VHH10:
   i) the CDR1 sequence of RYAMG (SEQ ID NO: 8), the CDR2 sequence of AISWSGSSAGYGDSVKG (SEQ ID NO: 37), and the CDR3 sequence of DPFNQGY (SEQ ID NO: 68);
   ii) the CDR1 sequence of GFTFTRY (SEQ ID NO: 17), the CDR2 sequence of SWSGSS (SEQ ID NO: 47) or WSGS (SEQ ID NO: 267), and the CDR3 sequence of DPFNQGY (SEQ ID NO: 79) or PFNQG (SEQ ID NO: 280);
   iii) the CDR1 sequence of GFTFTRYA (SEQ ID NO: 27), the CDR2 sequence of ISWSGSSA (SEQ ID NO: 57), and the CDR3 sequence of AADPFNQGY (SEQ ID NO: 90);
   iv) the CDR1 sequence of GFTFTRYAMG (SEQ ID NO: 161), the CDR2 sequence of AISWSGSSAGYGDSVKG (SEQ ID NO: 191), and the CDR3 sequence of DPFNQGY (SEQ ID NO: 222);
   v) the CDR1 sequence of TRYAMG (SEQ ID NO: 171), the CDR2 sequence of FVAAISWSGSSAG (SEQ ID NO: 201), and the CDR3 sequence of AADPFNQG (SEQ ID NO: 233); or
   vi) the CDR1 sequence of GFTFTRYAMG (SEQ ID NO: 181), the CDR2 sequence of AISWSGSSAG (SEQ ID NO: 211), and the CDR3 sequence of DPFNQGY (SEQ ID NO: 244);
j) VHH11:
   i) the CDR1 sequence of FTTYRMG (SEQ ID NO: 258) or TYRMG (SEQ ID NO: 259), the CDR2 sequence of AIRWSGGRTLYADSVKG (SEQ ID NO: 38), and the CDR3 sequence of DLAEYSGTYSSPADSPAGYDY (SEQ ID NO: 69);
   ii) the CDR1 sequence of GRTFTTY (SEQ ID NO: 18), the CDR2 sequence of RWSGGR (SEQ ID NO: 48) or WSGG (SEQ ID NO: 268), and the CDR3 sequence of DLAEYSGTYSSPADSPAGYDY (SEQ ID NO: 80) or LAEYSGTYSSPADSPAGYD (SEQ ID NO: 281);
   iii) the CDR1 sequence of GRTFTTYR (SEQ ID NO: 28), the CDR2 sequence of IRWSGGRT (SEQ ID NO: 58), and the CDR3 sequence of AADLAEYSGTYSSPADSPAGYDY (SEQ ID NO: 91);
   iv) the CDR1 sequence of GRTFTTYRMG (SEQ ID NO: 162), the CDR2 sequence of AIRWSGGRTLYADSVKG (SEQ ID NO: 192), and the CDR3 sequence of DLAEYSGTYSSPADSPAGYDY (SEQ ID NO: 223);
   v) the CDR1 sequence of TTYRMG (SEQ ID NO: 172), the CDR2 sequence of FVAAIRWSGGRTL (SEQ ID NO: 202), and the CDR3 sequence of AADLAEYSGTYSSPADSPAGYD (SEQ ID NO: 234); or
   vi) the CDR1 sequence of GRTFTTYRMG (SEQ ID NO: 182), the CDR2 sequence of AIRWSGGRTL (SEQ ID NO: 212), and the CDR3 sequence of DLAEYSGTYSSPADSPAGYDY (SEQ ID NO: 245); and
k) VHH12:
   i) the CDR1 sequence of FNTYAMG (SEQ ID NO: 9), the CDR2 sequence of SITWNGGSTSYADSVKG (SEQ ID NO: 39), and the CDR3 sequence of ARYYVSGTYFPANY (SEQ ID NO: 70);
   ii) the CDR1 sequence of GRTLSFNTY (SEQ ID NO: 19), the CDR2 sequence of TWNGGS (SEQ ID NO: 49) or WNGG (SEQ ID NO: 269), and the CDR3 sequence of ARYYVSGTYFPANY (SEQ ID NO: 81) or RYYVSGTYFPAN (SEQ ID NO: 282);
   iii) the CDR1 sequence of GRTLSFNTYA (SEQ ID NO: 29), the CDR2 sequence of ITWNGGST (SEQ ID NO: 59), and the CDR3 sequence of AAARYYVSGTYFPANY (SEQ ID NO: 92);
   iv) the CDR1 sequence of GRTLSFNTYAMG (SEQ ID NO: 163), the CDR2 sequence of SITWNGGSTSYADSVKG (SEQ ID NO: 193), and the CDR3 sequence of ARYYVSGTYFPANY (SEQ ID NO: 224);
   v) the CDR1 sequence of SFNTYAMG (SEQ ID NO: 173), the CDR2 sequence of FVASITWNGGSTS (SEQ ID NO: 203), and the CDR3 sequence of AAARYYVSGTYFPAN (SEQ ID NO: 235); or
   vi) the CDR1 sequence of GRTLSFNTYAMG (SEQ ID NO: 183), the CDR2 sequence of SITWNGGSTS (SEQ ID NO: 213), and the CDR3 sequence of ARYYVSGTYFPANY (SEQ ID NO: 246).

56. The method of any one of embodiments 1 to 34, wherein the VHH domain is comprised of a germline sequence of

(SEQ ID NO: 285)
QVQLVESGGGLVQAGGSLKLACAAPGLTFSSYRMGWFRQAPGQEREFVA
AIDWNGRGTYYRYYADSVKGRSTISRDNAKNTMYLQMNSLKPEDTAVYY
CA,
(SEQ ID NO: 286)
EVQVVESGGGLVQAGGSLKLACAAPGLTFSSYRMGWFRQAPGQEREFVA
AIDWNGRGTYYRYYADSVKGRSTISRDNAKNTVYLQMNSLKPEDTAVYY
CA,
(SEQ ID NO: 288)
EVQVVESGGGLVQAGGSLRLSCAVSGTSVSSNAMGWYRQAPGKQREWVG
FIDRIATTTIATSVKGRFAITRDNAKNTVYLQMSGLKPEDTAVYYCN,
(SEQ ID NO: 289)
QVQLVESGGGLVQAGGSLRLSCAASGRTFSSYAMGWFRQAPGKEREFVA
AITWNGGTTYYADSVKGRFTISRDNAKNTVYLQMNSLKPEDTAVYYCA
A,
(SEQ ID NO: 290)
EVQLVESGGGLVQAGGSLRLSCAVSGSSVSSDAMGWYRQAPGNQRAWVA
FISGGGTTTYADSVKGRFTISRDNTKNTVYLHMNSLKPEDTAVYYCN,
(SEQ ID NO: 291)
EVQVVESGGGLVQAGGSLRLACVASRSIGSINVMGWYRQAPGKQRDLVA
RITGGGSTHYAESVKGRFTISRDNAKNTVYLQMNSLEPEDTAVYYC,
(SEQ ID NO: 292)
QVQLVESGGGLVQAGGSLRLSCAVSGRTFSTYRMGWFRQAPGKERSFVA
AISWSGGSTTYADPVKGRFTISRDNAKNTVYLRMNSLKPEDTAVYYCN,
(SEQ ID NO: 293)
EVQVVESGGGLVQAGGSLRLSCAASGFTFTRYAMGWFRQAPGKERSFVA
AISWSGSSAGYGDSVKGRFTISRDNAKNTLYLQMNSLKPEDTAVYYCA,
(SEQ ID NO: 294)
EVQVVESGGGLVQAGGSLRLSCAASGRTFTTYRMGWFRQAPGKEREFVA
AIRWSGGRTLYADSVKGRFTISRDNAKNTAYLQMNNLRPEDTAVYYCA
A,
or
(SEQ ID NO: 295)
QVQLVETGGGLVQAGDSLRLSCAASGRTLSFNTYAMGWFRQAPGKEREF
VASITWNGGSTSYADSVKGRFTITRDNAKNTATLRMNSLQPDDTAVYYC
AA.

57. The method of any one of embodiments 1 to 34 and 47 to 56, wherein the VHH domain is comprised of a J segment, wherein the J segment comprises the sequence of

(SEQ ID NO: 296)
DYWGQGTQVTVSS,
(SEQ ID NO: 297)
EYATWGQGTQVTVSS,
(SEQ ID NO: 298)
YWGQGTQVTVSS,
(SEQ ID NO: 299)
WGQGTQVTVSS,
(SEQ ID NO: 300)
DYDYWGQGTQVTVSS,
(SEQ ID NO: 301)
WGQGTLVTVSS,
(SEQ ID NO: 302)
YDYWGQGTQVTVSS,
or
(SEQ ID NO: 303)
NYWGQGTQVTVSS.

58. The method of any one of embodiments 1 to 34, wherein the VHH domain is comprised of a sequence of

(SEQ ID NO: 93)
QVQLVESGGGLVQAGGSLKLACAAPGLTFSSYRMGWFRQAPGQEREFVA
AIDWNGRGTYYRYYADSVKGRSTISRDNAKNTMYLQMNSLKPEDTAVYY
CAAGSIDLNWYGGMDYWGQGTQVTVSS,
(SEQ ID NO: 94)
EVQVVESGGGLVQAGGSLKLACAAPGLTFSSYRMGWFRQAPGQEREFVA
AIDWNGRGTYYRYYADSVKGRSTISRDNAKNTVYLQMNSLKPEDTAVYY
CAATTVLTDPRVLNEYATWGQGTQVTVSS,
(SEQ ID NO: 95)
EVQVVESGGGLVQAGGSLRLSCAVSGTSVSSNAMGWYRQAPGKQREWVG
FIDRIATTTIATSVKGRFAITRDNAKNTVYLQMSGLKPEDTAVYYCNHP
LTARWGQGTQVTVSS,
(SEQ ID NO: 96)
QVQLVESGGGLVQAGGSLRLSCAASGRTFSSYAMGWFRQAPGKEREFVA
AITWNGGTTYYADSVKGRFTISRDNAKNTVYLQMNSLKPEDTAVYYCAA
DPFNQGYWGQGTQVTVSS,
(SEQ ID NO: 97)
EVQLVESGGGLVQAGGSLRLSCAVSGSSVSSDAMGWYRQAPGNQRAWVA
FISGGGTTTYADSVKGRFTISRDNTKNTVYLHMNSLKPEDTAVYYCNHP
LTSRWGQGTQVTVSS,
(SEQ ID NO: 98)
EVQVVESGGGLVQAGGSLRLACVASRSIGSINVMGWYRQAPGKQRDLVA
RITGGGSTHYAESVKGRFTISRDNAKNTVYLQMNSLEPEDTAVYYCASM
VNPIITAWGTIGVREIPDYDYWGQGTQVTVSS,
(SEQ ID NO: 99)
QVQLVESGGGLVQAGGSLRLSCAVSGRTFSTYRMGWFRQAPGKERSFVA
AISWSGGSTTYADPVKGRFTISRDNAKNTVYLRMNSLKPEDTAVYYCND
QRGYWGQGTLVTVSS,
(SEQ ID NO: 100)
EVQVVESGGGLVQAGGSLRLSCAASGFTFTRYAMGWFRQAPGKERSFVA
AISWSGSSAGYGDSVKGRFTISRDNAKNTLYLQMNSLKPEDTAVYYCAA
DPFNQGYWGQGTQVTVSS,
(SEQ ID NO: 101)
EVQVVESGGGLVQAGGSLRLSCAASGRTFTTYRMGWFRQAPGKEREFVA
AIRWSGGRTLYADSVKGRFTISRDNAKNTAYLQMNNLRPEDTAVYYCAA
DLAEYSGTYSSPADSPAGYDYWGQGTQVTVSS,
or
(SEQ ID NO: 102)
QVQLVETGGGLVQAGDSLRLSCAASGRTLSFNTYAMGWFRQAPGKEREF
VASITWNGGSTSYADSVKGRFTITRDNAKNTATLRMNSLQPDDTAVYYC
AAARYYVSGTYFPANYWGQGTQVTVSS.

59. The method of any one of embodiments 1 to 34, wherein the VHH domain comprises a complementarity determining region 1 (CDR1) comprising the amino acid sequence of SYRMG (SEQ ID NO: 1), SDAMG (SEQ ID NO: 5), TYRMG (SEQ ID NO: 7), FTTYRMG (SEQ ID NO: 258), TYRMG (SEQ ID NO: 259), or FNTYAMG (SEQ ID NO: 9).

60. The method of any one of embodiments 1 to 34, wherein the VHH domain comprises a complementarity determining region 1 (CDR1) comprising the amino acid sequence of GLTFSSY (SEQ ID NO: 10), GSSVSSD (SEQ ID NO: 14), GRTFSSY (SEQ ID NO: 13), GRTFSTY (SEQ ID NO: 16), GRTFTTY (SEQ ID NO: 18), or GRTLSFNTY (SEQ ID NO: 19).

61. The method of any one of embodiments 1 to 34, wherein the VHH domain comprises a complementarity determining region 1 (CDR1) comprising the amino acid sequence of (i) GLTFSSYR (SEQ ID NO: 20), GSSVSSDA (SEQ ID NO: 24), GRTFSTYR (SEQ ID NO: 26), GRTFTTYR (SEQ ID NO: 28), or GRTLSFNTYA (SEQ ID NO: 29); (ii) GLTFSSYRMG (SEQ ID NO: 154), GSSVSSDAMG (SEQ ID NO: 158), GRTFSTYRMG (SEQ ID NO: 160), GRTFTTYRMG (SEQ ID NO: 162), or GRTLSFNTYAMG (SEQ ID NO: 163); (iii) SSYRMG (SEQ ID NO: 164), SSDAMG (SEQ ID NO: 168), STYRMG (SEQ ID NO: 170), TTYRMG (SEQ ID NO: 172), or SFNTYAMG (SEQ ID NO: 173); or (iv) GLTFSSYRMG (SEQ ID NO: 174), GSSVSSDAMG (SEQ ID NO: 178), GRTFSTYRMG (SEQ ID NO: 180), GRTFTTYRMG (SEQ ID NO: 182), or GRTLSFNTYAMG (SEQ ID NO: 183).

62. The method of any one of embodiments 1 to 34 and 59 to 61, wherein the VHH domain comprises a complementarity determining region 2 (CDR2) comprising the amino acid sequence of AIDWNGRGTYYRYYADSVKG (SEQ ID NO: 30), FISGGGTTTYADSVKG (SEQ ID NO: 34), AISWSGGSTTYADPVKG (SEQ ID NO: 36), AIRWSGGRTLYADSVKG (SEQ ID NO: 38), or SITWNGGSTSYADSVKG (SEQ ID NO: 39).

63. The method of any one of embodiments 1 to 34 and 59 to 61, wherein the VHH domain comprises a complementarity determining region 2 (CDR2) comprising the amino acid sequence of DWNGRGTYY (SEQ ID NO: 40), WNGRGTY (SEQ ID NO: 260), SGGGT (SEQ ID NO: 44), GGG (SEQ ID NO: 264, SWSGGS (SEQ ID NO: 46), WSGG (SEQ ID NO: 266), RWSGGR (SEQ ID NO: 48), WSGG (SEQ ID NO: 268), TWNGGS (SEQ ID NO: 49), or WNGG (SEQ ID NO: 269).

64. The method of any one of embodiments 1 to 34 and 59 to 61, wherein the VHH domain comprises a complementarity determining region 2 (CDR2) comprising the amino acid sequence of (i) IDWNGRGTYY (SEQ ID NO: 50), IDWNGRGTYYR (SEQ ID NO: 270), ISGGGTT (SEQ ID NO: 54), ISWSGGST (SEQ ID NO: 56), IRWSGGRT (SEQ ID NO: 58), or ITWNGGST (SEQ ID NO: 59); (ii) AIDWNGRGTYYRYYADSVKG (SEQ ID NO: 184), FISGGGTTTYADSVKG (SEQ ID NO: 188), AISWSGGSTTYADPVKG (SEQ ID NO: 190), AIRWSGGRTLYADSVKG (SEQ ID NO: 192), or SITWNGGSTSYADSVKG (SEQ ID NO: 193); (iii) FVAAIDWNGRGTYYRY (SEQ ID NO: 194), WVAFISGGGTTT (SEQ ID NO: 198), FVAAISWSGGSTT (SEQ ID NO: 200), FVAAIRWSGGRTL (SEQ ID NO: 202), or FVASITWNGGSTS (SEQ ID NO: 203); or (iv) AIDWNGRGTYYRY (SEQ ID NO: 204), FISGGGTTT (SEQ ID NO: 208), AISWSGGSTT (SEQ ID NO: 210), AIRWSGGRTL (SEQ ID NO: 212), or SITWNGGSTS (SEQ ID NO: 213).

65. The method of any one of embodiments 1 to 34 and 59 to 64, wherein the VHH domain comprises a complementarity determining region 3 (CDR3) comprising the amino acid sequence of TTVLTDPRVLNEYAT (SEQ ID NO: 61), PLTSR (SEQ ID NO: 65), DQRGY (SEQ ID NO: 67), QRGY (SEQ ID NO: 271), DLAEYSGTYSSPADSPAGYDY (SEQ ID NO: 69), or ARYYVSGTYFPANY (SEQ ID NO: 70).

66. The method of any one of embodiments 1 to 34 and 59 to 64, wherein the VHH domain comprises a complementarity determining region 3 (CDR3) comprising the amino acid sequence of TTVLTDPRVLNEYAT (SEQ ID NO: 72), TVLTDPRVLNEYA (SEQ ID NO: 273), PLTSR (SEQ ID NO: 76), LTS (SEQ ID NO: 277), DQRGY (SEQ ID NO: 78), RG (SEQ ID NO: 279), DLAEYSGTYSSPADSPAGYDY (SEQ ID NO: 80), LAEYSGTYSSPADSPAGYD (SEQ ID NO: 281), ARYYVSGTYFPANY (SEQ ID NO: 81), or RYYVSGTYFPAN (SEQ ID NO: 282).

67. The method of any one of embodiments 1 to 34 and 59 to 64, wherein the VHH domain comprises a complementarity determining region 3 (CDR3) comprising the amino acid sequence of (i) CAATTVLTDPRVLNEYAT (SEQ ID NO: 83), AATTVLTDPRVLNEYAT (SEQ ID NO: 284), NHPLTSR (SEQ ID NO: 87), NDQRGY (SEQ ID NO: 89), AADLAEYSGTYSSPADSPAGYDY (SEQ ID NO: 91), or AAARYYVSGTYFPANY (SEQ ID NO: 92); (ii) TTVLTDPRVLNEYAT (SEQ ID NO: 215), PLTSR (SEQ ID NO: 219), QRGY (SEQ ID NO: 221), DLAEYSGTYSSPADSPAGYDY (SEQ ID NO: 223), or ARYYVSGTYFPANY (SEQ ID NO: 224); (iii) AATTVLTDPRVLNEYA (SEQ ID NO: 226), NHPLTS (SEQ ID NO: 230), NDQRG (SEQ ID NO: 232), AADLAEYSGTYSSPADSPAGYD (SEQ ID NO: 234), or AAARYYVSGTYFPAN (SEQ ID NO: 235); or (iv) TTVLTDPRVLNEYAT (SEQ ID NO: 237), PLTSR (SEQ ID NO: 241), QRGY (SEQ ID NO: 243), DLAEYSGTYSSPADSPAGYDY (SEQ ID NO: 245), or ARYYVSGTYFPANY (SEQ ID NO: 246), including wherein optionally the VHH domain comprises a CDR1 sequence, a CDR2 sequence, and a CDR3 sequence of the VHH domain selected from the group consisting of:
a) VHH1:
   i) the CDR1 sequence of SYRMG (SEQ ID NO: 1), the CDR2 sequence of AIDWNGRGTYYRYYADSVKG (SEQ ID NO: 30), and the CDR3 sequence of GSIDLNWYGGMDY (SEQ ID NO: 60);
   ii) the CDR1 sequence of GLTFSSY (SEQ ID NO: 10), the CDR2 sequence of DWNGRGTYY (SEQ ID NO: 40) or WNGRGTY (SEQ ID NO: 260), and the CDR3 sequence of GSIDLNWYGGMDY (SEQ ID NO: 71) or SIDLNWYGGMD (SEQ ID NO: 272);
   iii) the CDR1 sequence of GLTFSSYR (SEQ ID NO: 20), the CDR2 sequence of IDWNGRGTYY (SEQ ID NO: 50) or IDWNGRGTYYR (SEQ ID NO: 270), and the CDR3 sequence of CAAGSIDLNWYGGMDY (SEQ ID NO: 82) or AAGSIDLNWYGGMDY (SEQ ID NO: 283);
   iv) the CDR1 sequence of GLTFSSYRMG (SEQ ID NO: 154), the CDR 2 sequence of AIDWNGRGTYYRYYADSVKG (SEQ ID NO: 184), and the CDR3 sequence of GSIDLNWYGGMDY (SEQ ID NO: 214);
   v) the CDR1 sequence of SSYRMG (SEQ ID NO: 164), the CDR2 sequence of FVAAIDWNGRGTYYRY (SEQ ID NO: 194), and the CDR3 sequence of AAGSIDLNWYGGMD (SEQ ID NO: 225); or
   vi) the CDR1 sequence of GLTFSSYRMG (SEQ ID NO: 174), the CDR2 sequence of AIDWNGRGTYYRY (SEQ ID NO: 204), and the CDR3 sequence of GSIDLNWYGGMDY (SEQ ID NO: 236);
b) VHH2:
   i) the CDR1 sequence of SYRMG (SEQ ID NO: 1), the CDR2 sequence of AIDWNGRGTYYRYYADSVKG (SEQ ID NO: 30), and the CDR3 sequence of TTVLTDPRVLNEYAT (SEQ ID NO: 61);
   ii) the CDR1 sequence of GLTFSSY (SEQ ID NO: 10), the CDR2 sequence of DWNGRGTYY (SEQ ID NO: 40) or WNGRGTY (SEQ ID NO: 260), and the CDR3 sequence of TTVLTDPRVLNEYAT (SEQ ID NO: 72) or TVLTDPRVLNEYA (SEQ ID NO: 273);
   iii) the CDR1 sequence of GLTFSSYR (SEQ ID NO: 20), the CDR2 sequence of IDWNGRGTYY (SEQ ID NO: 50) or IDWNGRGTYYR (SEQ ID NO: 270), and the CDR3 sequence of CAATTVLTDPRVLNEYAT (SEQ ID NO: 83) or AATTVLTDPRVLNEYAT (SEQ ID NO: 284);
   iv) the CDR1 sequence of GLTFSSYRMG (SEQ ID NO: 154), the CDR2 sequence of AIDWNGRGTYYRYYADSVKG (SEQ ID NO: 184), and the CDR3 sequence of TTVLTDPRVLNEYAT (SEQ ID NO: 215);
   v) the CDR1 sequence of SSYRMG (SEQ ID NO: 164), the CDR2 sequence of FVAAIDWNGRGTYYRY (SEQ ID NO: 194), and the CDR3 sequence of AATTVLTDPRVLNEYA (SEQ ID NO: 226); or
   vi) the CDR1 sequence of GLTFSSYRMG (SEQ ID NO: 174), the CDR2 sequence of AIDWNGRGTYYRY (SEQ ID NO: 204), and the CDR3 sequence of TTVLTDPRVLNEYAT (SEQ ID NO: 237);
c) VHH3:
   i) the CDR1 sequence of INVMG (SEQ ID NO: 2), the CDR2 sequence of RINGGGITHYAESVKG (SEQ ID NO: 31), and the CDR3 sequence of DVFGSSGYVETY (SEQ ID NO: 62);
   ii) the CDR1 sequence of GSIFSIN (SEQ ID NO: 11), the CDR2 sequence of NGGGI (SEQ ID NO: 41) or GGG (SEQ ID NO: 261), and the CDR3 sequence of DVFGSSGYVETY (SEQ ID NO: 73) or VFGSSGYVET (SEQ ID NO: 274);
   iii) the CDR1 sequence of GSIFSINV (SEQ ID NO: 21), the CDR2 sequence of INGGGIT (SEQ ID NO: 51), and the CDR3 sequence of KADVFGSSGYVETY (SEQ ID NO: 84);
   iv) the CDR1 sequence of GSIFSINVMG (SEQ ID NO: 155), the CDR2 sequence of RINGGGITHYAESVKG (SEQ ID NO: 185), and the CDR3 sequence of DVFGSSGYVETY (SEQ ID NO: 216);
   v) the CDR1 sequence of SINVMG (SEQ ID NO: 165), the CDR2 sequence of LVARINGGGITH (SEQ ID NO: 195), and the CDR3 sequence of KADVFGSSGYVET (SEQ ID NO: 227); or
   vi) the CDR1 sequence of GSIFSINVMG (SEQ ID NO: 175), the CDR2 sequence of RINGGGITH (SEQ ID NO: 205), and the CDR3 sequence of DVFGSSGYVETY (SEQ ID NO: 238);
d) VHH4:
   i) the CDR1 sequence of SNAMG (SEQ ID NO: 3), the CDR2 sequence of FIDRIATTTIATSVKG (SEQ ID NO: 32), and the CDR3 sequence of PLTAR (SEQ ID NO: 63);
   ii) the CDR1 sequence of GTSVSSN (SEQ ID NO: 12), the CDR2 sequence of DRIAT (SEQ ID NO: 42) or RIA (SEQ ID NO: 262), and the CDR3 sequence of PLTAR (SEQ ID NO: 74) or LTA (SEQ ID NO: 275);
   iii) the CDR1 sequence of GTSVSSNA (SEQ ID NO: 22), the CDR2 sequence of IDRIATT (SEQ ID NO: 52), and the CDR3 sequence of NHPLTAR (SEQ ID NO: 85);
   iv) the CDR1 sequence of GTSVSSNAMG (SEQ ID NO: 156), the CDR2 sequence of FIDRIATTTIATSVKG (SEQ ID NO: 186), and the CDR3 sequence of PLTAR (SEQ ID NO: 217);
   v) the CDR1 sequence of SSNAMG (SEQ ID NO: 166), the CDR2 sequence of WVGFIDRIATTT (SEQ ID NO: 196), and the CDR3 sequence of NHPLTA (SEQ ID NO: 228); or
   vi) the CDR1 sequence of GTSVSSNAMG (SEQ ID NO: 176), the CDR2 sequence of FIDRIATTT (SEQ ID NO: 206), and the CDR3 sequence of PLTAR (SEQ ID NO: 239);
e) VHH5:
   i) the CDR1 sequence of SYAMG (SEQ ID NO: 4), the CDR2 sequence of AITWNGGTTYYADSVKG (SEQ ID NO: 33), and the CDR3 sequence of DPFNQGY (SEQ ID NO: 64);
   ii) the CDR1 sequence of GRTFSSY (SEQ ID NO: 13), the CDR2 sequence of TWNGGT (SEQ ID NO: 43) or WNGG (SEQ ID NO: 263), and the CDR3 sequence of DPFNQGY (SEQ ID NO: 75) or PFNQG (SEQ ID NO: 276);
   iii) the CDR1 sequence of GRTFSSYA (SEQ ID NO: 23), the CDR2 sequence of ITWNGGTT (SEQ ID NO: 53), and the CDR3 sequence of AADPFNQGY (SEQ ID NO: 86);
   iv) the CDR1 sequence of GRTFSSYAMG (SEQ ID NO: 157), the CDR2 sequence of AITWNGGTTYYADSVKG (SEQ ID NO: 187), and the CDR3 sequence of DPFNQGY (SEQ ID NO: 218);
   v) the CDR1 sequence of SSYAMG (SEQ ID NO: 167), the CDR2 sequence of FVAAITWNGGTTY (SEQ ID NO: 197), and the CDR3 sequence of AADPFNQG (SEQ ID NO: 229); or
   vi) the CDR1 sequence of GRTFSSYAMG (SEQ ID NO: 177), the CDR2 sequence of AITWNGGTTY (SEQ ID NO: 207), and the CDR3 sequence of DPFNQGY (SEQ ID NO: 240);
f) VHH6:
   i) the CDR1 sequence of SDAMG (SEQ ID NO: 5), the CDR2 sequence of FISGGGTTTYADSVKG (SEQ ID NO: 34), and the CDR3 sequence of PLTSR (SEQ ID NO: 65);
   ii) the CDR1 sequence of GSSVSSD (SEQ ID NO: 14), the CDR2 sequence of SGGGT (SEQ ID NO: 44) or GGG (SEQ ID NO: 264), and the CDR3 sequence of PLTSR (SEQ ID NO: 76) or LTS (SEQ ID NO: 277);
   iii) the CDR1 sequence of GSSVSSDA (SEQ ID NO: 24), the CDR2 sequence of ISGGGTT (SEQ ID NO: 54), and the CDR3 sequence of NHPLTSR (SEQ ID NO: 87);
   iv) the CDR1 sequence of GSSVSSDAMG (SEQ ID NO: 158), the CDR2 sequence of FISGGGTTTYADSVKG (SEQ ID NO: 188), and the CDR3 sequence of PLTSR (SEQ ID NO: 219);
   v) the CDR1 sequence of SSDAMG (SEQ ID NO: 168), the CDR2 sequence of WVAFISGGGTTT (SEQ ID NO: 198), and the CDR3 sequence of NHPLTS (SEQ ID NO: 230); or
   vi) the CDR1 sequence of GSSVSSDAMG (SEQ ID NO: 178), the CDR2 sequence of FISGGGTTT (SEQ ID NO: 208), and the CDR3 sequence of PLTSR (SEQ ID NO: 241);
g) VHH7:
   i) the CDR1 sequence of INVMG (SEQ ID NO: 6), the CDR2 sequence of RITGGGSTHYAESVKG (SEQ ID NO: 35), and the CDR3 sequence of MVNPIITAWGTIGVREIPDYDY (SEQ ID NO: 66);
   ii) the CDR1 sequence of RSIGSIN (SEQ ID NO: 15), the CDR2 sequence of TGGGS (SEQ ID NO: 45) or GGG (SEQ ID NO: 265), and the CDR3 sequence of MVNPIITAWGTIGVREIPDYDY (SEQ ID NO: 77) or VNPIITAWGTIGVREIPDYD (SEQ ID NO: 278);
   iii) the CDR1 sequence of RSIGSINV (SEQ ID NO: 25), the CDR2 sequence of ITGGGST (SEQ ID NO: 55), and the CDR3 sequence of ASMVNPIITAWGTIGVREIPDYDY (SEQ ID NO: 88);
   iv) the CDR1 sequence of RSIGSINVMG (SEQ ID NO: 159), the CDR2 sequence of RITGGGSTHYAESVKG (SEQ ID NO: 189), and the CDR3 sequence of MVNPIITAWGTIGVREIPDYDY (SEQ ID NO: 220);
   v) the CDR1 sequence of SINVMG (SEQ ID NO: 169), the CDR2 sequence of LVARITGGGSTH (SEQ ID NO: 199), and the CDR3 sequence of ASMVNPIITAWGTIGVREIPDYD (SEQ ID NO: 231); or
   vi) the CDR1 sequence of RSIGSINVMG (SEQ ID NO: 179), the CDR2 sequence of RITGGGSTH (SEQ ID NO: 209), and the CDR3 sequence of MVNPIITAWGTIGVREIPDYDY (SEQ ID NO: 242);
h) VHH9:
   i) the CDR1 sequence of TYRMG (SEQ ID NO: 7), the CDR2 sequence of AISWSGGSTTYADPVKG (SEQ ID NO: 36), and the CDR3 sequence of DQRGY (SEQ ID NO: 67) or QRGY (SEQ ID NO: 271);
   ii) the CDR1 sequence of GRTFSTY (SEQ ID NO: 16), the CDR2 sequence of SWSGGS (SEQ ID NO: 46) or WSGG (SEQ ID NO: 266), and the CDR3 sequence of DQRGY (SEQ ID NO: 78) or RG (SEQ ID NO: 279);
   iii) the CDR1 sequence of GRTFSTYR (SEQ ID NO: 26), the CDR2 sequence of ISWSGGST (SEQ ID NO: 56), and the CDR3 sequence of NDQRGY (SEQ ID NO: 89);
   iv) the CDR1 sequence of GRTFSTYRMG (SEQ ID NO: 160), the CDR2 sequence of AISWSGGSTTYADPVKG (SEQ ID NO: 190), and the CDR3 sequence of QRGY (SEQ ID NO: 221);
   v) the CDR1 sequence of STYRMG (SEQ ID NO: 170), the CDR2 sequence of FVAAISWSGGSTT (SEQ ID NO: 200), and the CDR3 sequence of NDQRG (SEQ ID NO: 232); or
   vi) the CDR1 sequence of GRTFSTYRMG (SEQ ID NO: 180), the CDR2 sequence of AISWSGGSTT (SEQ ID NO: 210), and the CDR3 sequence of QRGY (SEQ ID NO: 243);
i) VHH10:
   i) the CDR1 sequence of RYAMG (SEQ ID NO: 8), the CDR2 sequence of AISWSGSSAGYGDSVKG (SEQ ID NO: 37), and the CDR3 sequence of DPFNQGY (SEQ ID NO: 68);
   ii) the CDR1 sequence of GFTFTRY (SEQ ID NO: 17), the CDR2 sequence of SWSGSS (SEQ ID NO: 47) or WSGS (SEQ ID NO: 267), and the CDR3 sequence of DPFNQGY (SEQ ID NO: 79) or PFNQG (SEQ ID NO: 280);
   iii) the CDR1 sequence of GFTFTRYA (SEQ ID NO: 27), the CDR2 sequence of ISWSGSSA (SEQ ID NO: 57), and the CDR3 sequence of AADPFNQGY (SEQ ID NO: 90);
   iv) the CDR1 sequence of GFTFTRYAMG (SEQ ID NO: 161), the CDR2 sequence of AISWSGSSAGYGDSVKG (SEQ ID NO: 191), and the CDR3 sequence of DPFNQGY (SEQ ID NO: 222);
   v) the CDR1 sequence of TRYAMG (SEQ ID NO: 171), the CDR2 sequence of FVAAISWSGSSAG (SEQ ID NO: 201), and the CDR3 sequence of AADPFNQG (SEQ ID NO: 233); or
   vi) the CDR1 sequence of GFTFTRYAMG (SEQ ID NO: 181), the CDR2 sequence of AISWSGSSAG (SEQ ID NO: 211), and the CDR3 sequence of DPFNQGY (SEQ ID NO: 244);
j) VHH11:
   i) the CDR1 sequence of FTTYRMG (SEQ ID NO: 258) or TYRMG (SEQ ID NO: 259), the CDR2 sequence of AIRWSGGRTLYADSVKG (SEQ ID NO: 38), and the CDR3 sequence of DLAEYSGTYSSPADSPAGYDY (SEQ ID NO: 69);
   ii) the CDR1 sequence of GRTFTTY (SEQ ID NO: 18), the CDR2 sequence of RWSGGR (SEQ ID NO: 48) or WSGG (SEQ ID NO: 268), and the CDR3 sequence of DLAEYSGTYSSPADSPAGYDY (SEQ ID NO: 80) or LAEYSGTYSSPADSPAGYD (SEQ ID NO: 281);
   iii) the CDR1 sequence of GRTFTTYR (SEQ ID NO: 28), the CDR2 sequence of IRWSGGRT (SEQ ID NO: 58), and the CDR3 sequence of AADLAEYSGTYSSPADSPAGYDY (SEQ ID NO: 91);
   iv) the CDR1 sequence of GRTFTTYRMG (SEQ ID NO: 162), the CDR2 sequence of AIRWSGGRTLYADSVKG (SEQ ID NO: 192), and the CDR3 sequence of DLAEYSGTYSSPADSPAGYDY (SEQ ID NO: 223);
   v) the CDR1 sequence of TTYRMG (SEQ ID NO: 172), the CDR2 sequence of FVAAIRWSGGRTL (SEQ ID NO: 202), and the CDR3 sequence of AADLAEYSGTYSSPADSPAGYD (SEQ ID NO: 234); or
   vi) the CDR1 sequence of GRTFTTYRMG (SEQ ID NO: 182), the CDR2 sequence of AIRWSGGRTL (SEQ ID NO: 212), and the CDR3 sequence of DLAEYSGTYSSPADSPAGYDY (SEQ ID NO: 245); and
k) VHH12:
   i) the CDR1 sequence of FNTYAMG (SEQ ID NO: 9), the CDR2 sequence of SITWNGGSTSYADSVKG (SEQ ID NO: 39), and the CDR3 sequence of ARYYVSGTYFPANY (SEQ ID NO: 70);
   ii) the CDR1 sequence of GRTLSFNTY (SEQ ID NO: 19), the CDR2 sequence of TWNGGS (SEQ ID NO: 49) or WNGG (SEQ ID NO: 269), and the CDR3 sequence of ARYYVSGTYFPANY (SEQ ID NO: 81) or RYYVSGTYFPAN (SEQ ID NO: 282);
   iii) the CDR1 sequence of GRTLSFNTYA (SEQ ID NO: 29), the CDR2 sequence of ITWNGGST (SEQ ID NO: 59), and the CDR3 sequence of AAARYYVSGTYFPANY (SEQ ID NO: 92);
   iv) the CDR1 sequence of GRTLSFNTYAMG (SEQ ID NO: 163), the CDR2 sequence of SITWNGGSTSYADSVKG (SEQ ID NO: 193), and the CDR3 sequence of ARYYVSGTYFPANY (SEQ ID NO: 224);
   v) the CDR1 sequence of SFNTYAMG (SEQ ID NO: 173), the CDR2 sequence of FVASITWNGGSTS (SEQ ID NO: 203), and the CDR3 sequence of AAARYYVSGTYFPAN (SEQ ID NO: 235); or
   vi) the CDR1 sequence of GRTLSFNTYAMG (SEQ ID NO: 183), the CDR2 sequence of SITWNGGSTS (SEQ ID NO: 213), and the CDR3 sequence of ARYYVSGTYFPANY (SEQ ID NO: 246).

68. The method of any one of embodiments 1 to 34, wherein the VHH domain is comprised of a germline sequence of

(SEQ ID NO: 286)
EVQVVESGGGLVQAGGSLKLACAAPGLTFSSYRMGWFRQAPGQEREFVA
AIDWNGRGTYYRYYADSVKGRSTISRDNAKNTVYLQMNSLKPEDTAVYY
CA,
(SEQ ID NO: 290)
EVQLVESGGGLVQAGGSLRLSCAVSGSSVSSDAMGWYRQAPGNQRAWVA
FISGGGTTTYADSVKGRFTISRDNTKNTVYLHMNSLKPEDTAVYYCN,
(SEQ ID NO: 292)
QVQLVESGGGLVQAGGSLRLSCAVSGRTFSTYRMGWFRQAPGKERSFVA
AISWSGGSTTYADPVKGRFTISRDNAKNTVYLRMNSLKPEDTAVYYCN,
(SEQ ID NO: 294)
EVQVVESGGGLVQAGGSLRLSCAASGRTFTTYRMGWFRQAPGKEREFVA
AIRWSGGRTLYADSVKGRFTISRDNAKNTAYLQMNNLRPEDTAVYYCA
A,
or
(SEQ ID NO: 295)
QVQLVETGGGLVQAGDSLRLSCAASGRTLSFNTYAMGWFRQAPGKEREF
VASITWNGGSTSYADSVKGRFTITRDNAKNTATLRMNSLQPDDTAVYYC
AA.

69. The method of any one of embodiments 1 to 34 and 59 to 68, wherein the VHH domain is comprised of a J segment, wherein the J segment comprises the sequence of EYATWGQGTQVTVSS (SEQ ID NO: 297), WGQGTLVTVSS (SEQ ID NO: 301), YDYWGQGTQVTVSS (SEQ ID NO: 302), or NYWGQGTQVTVSS (SEQ ID NO: 303).

70. The method of any one of embodiments 1 to 34, wherein the VHH domain is comprised of a sequence of

(SEQ ID NO: 94)
EVQVVESGGGLVQAGGSLKLACAAPGLTFSSYRMGWFRQAPGQEREFVA
AIDWNGRGTYYRYYADSVKGRSTISRDNAKNTVYLQMNSLKPEDTAVYY
CAATTVLTDPRVLNEYATWGQGTQVTVSS,
(SEQ ID NO: 98)
EVQLVESGGGLVQAGGSLRLSCAVSGSSVSSDAMGWYRQAPGNQRAWVA
FISGGGTTTYADSVKGRFTISRDNTKNTVYLHMNSLKPEDTAVYYCNHP
LTSRWGQGTQVTVSS,
(SEQ ID NO: 100)
QVQLVESGGGLVQAGGSLRLSCAVSGRTFSTYRMGWFRQAPGKERSFVA
AISWSGGSTTYADPVKGRFTISRDNAKNTVYLRMNSLKPEDTAVYYCND
QRGYWGQGTLVTVSS,
(SEQ ID NO: 102)
EVQVVESGGGLVQAGGSLRLSCAASGRTFTTYRMGWFRQAPGKEREFVA
AIRWSGGRTLYADSVKGRFTISRDNAKNTAYLQMNNLRPEDTAVYYCAA
DLAEYSGTYSSPADSPAGYDYWGQGTQVTVSS,
or
(SEQ ID NO: 103)
QVQLVETGGGLVQAGDSLRLSCAASGRTLSFNTYAMGWFRQAPGKEREF
VASITWNGGSTSYADSVKGRFTITRDNAKNTATLRMNSLQPDDTAVYYC
AAARYYVSGTYFPANYWGQGTQVTVSS.

71. A pIgR modulator comprising a VHH domain, wherein optionally the VHH domain binds to an extracellular domain of the pIgR, including a VHH domain that binds to an extracellular domain 1, an extracellular domain 2, an extracellular domain 1-2, an extracellular domain 3, an extracellular domain 2-3, an extracellular domain 4-5, or an extracellular domain 5 of pIgR, wherein optionally pIgR is human pIgR or mouse pIgR, wherein optionally the VHH domain does not detectably bind to the amino acid sequence of

(SEQ ID NO: 143)
EKAVADTRDQADGSRASVDSGSSEEQGGSSR,
(SEQ ID NO: 144)
EREIQNVGDQAQENRASGDAGSADGQSRSSSSK
or
(SEQ ID NO: 145)
EREIQNVRDQAQENRASGDAGSADGQSRSSSSK.

72. The pIgR modulator of embodiment 71, further comprising an agent.

73. The pIgR modulator of embodiment 71 or embodiment 72, wherein the agent is an antibody or fragment thereof, a peptide, a vaccine, a small molecule, a polynucleotide, a radioisotope, a toxin, an enzyme, an anticoagulant, a hormone, a cytokine, an anti-inflammatory molecule, an RNAi, an mRNA, a self-replicating RNA, an antibiotic, or an antibody-antibiotic conjugate.

74. The pIgR modulator of embodiment 73, wherein the agent is an antibiotic, an antibody or fragment thereof, a peptide or a vaccine.

75. The pIgR modulator of embodiment 74, wherein the antibiotic is selected from the group consisting of a macrolide antibiotic, a fluoroquinolone, a tetracycline, amoxicillin, ceftriaxone, penicillin G, linezolid, moxifloxacin, and azithromycin.

76. The pIgR modulator of any one of embodiments 73 to 75, wherein the radioisotope is ¹⁸F, ⁹⁹Tc, ¹¹¹In, ¹²³I, ²⁰¹Tl, ¹³³Xe, ¹¹C, ¹³N, ¹⁵O, ¹⁸F, ⁶²Cu, ⁶⁴Cu, ¹²⁴I, ⁷⁶Br, ⁸²Rb, ⁸⁹Zr or ⁶⁸Ga.

77. The pIgR modulator of any one of embodiments 73 to 75, wherein the molecule comprises a VHH domain genetically fused or chemically conjugated to the agent.

78. The pIgR modulator of any one of embodiments 72 to 77, wherein the molecule further comprises a linker between the VHH domain and the agent.

79. The pIgR modulator of embodiment 78, wherein the linker is a polypeptide.

80. The pIgR modulator of embodiment 79, wherein the linker is a flexible linker comprising a sequence selected from the group consisting of EPKTPKPQPQPQLQPQPNPTTESKSPK (SEQ ID NO: 130), (EAAAK)n (SEQ ID NO: 147), (GGGGS)n (SEQ ID NO: 148) and (GGGS)n (SEQ ID NO: 149), wherein n is an integer from 1 to 20.

81. The pIgR modulator of any one of embodiments 71 to 80, wherein the VHH domain is chemically-conjugated to the agent.

82. The method of any one of embodiments 71 to 80, wherein the VHH domain is non-covalently bound to the molecule.

83. The pIgR modulator of any one of embodiments 71 to 82, wherein the VHH domain comprises a complementarity determining region 1 (CDR1) comprising the amino acid sequence of SYRMG (SEQ ID NO: 1), INVMG (SEQ ID NO: 2), SNAMG (SEQ ID NO: 3), SYAMG (SEQ ID NO: 4), SDAMG (SEQ ID NO: 5), INVMG (SEQ ID NO: 6), TYRMG (SEQ ID NO: 7), RYAMG (SEQ ID NO: 8), FTTYRMG (SEQ ID NO: 258), TYRMG (SEQ ID NO: 259), or FNTYAMG (SEQ ID NO: 9).

84. The pIgR modulator of any one of embodiments 71 to 82, wherein the VHH domain comprises a complementarity determining region 1 (CDR1) comprising the amino acid sequence of GLTFSSY (SEQ ID NO: 10), GSIFSIN (SEQ ID NO: 11), GTSVSSN (SEQ ID NO: 12), GSSVSSD (SEQ ID NO: 14), RSIGSIN (SEQ ID NO: 15), GRTFSSY (SEQ ID NO: 13), GRTFSTY (SEQ ID NO: 16), GFTFTRY (SEQ ID NO: 17), GRTFTTY (SEQ ID NO: 18), or GRTLSFNTY (SEQ ID NO: 19).

85. The pIgR modulator of any one of embodiments 71 to 82, wherein the VHH domain comprises a complementarity determining region 1 (CDR1) comprising the amino acid sequence of (i) GLTFSSYR (SEQ ID NO: 20), GSIFSINV (SEQ ID NO: 21), GTSVSSNA (SEQ ID NO: 22), GRTFSSYA (SEQ ID NO: 23), GSSVSSDA (SEQ ID NO: 24), RSIGSINV (SEQ ID NO: 25), GRTFSTYR (SEQ ID NO: 26), GFTFTRYA (SEQ ID NO: 27), GRTFTTYR (SEQ ID NO: 28), or GRTLSFNTYA (SEQ ID NO: 29); (ii) GLTFSSYRMG (SEQ ID NO: 154), GSIFSINVMG (SEQ ID NO: 155), GTSVSSNAMG (SEQ ID NO: 156), GRTFSSYAMG (SEQ ID NO: 157), GSSVSSDAMG (SEQ ID NO: 158), RSIGSINVMG (SEQ ID NO: 159), GRTFSTYRMG (SEQ ID NO: 160), GFTFTRYAMG (SEQ ID NO: 161), GRTFTTYRMG (SEQ ID NO: 162), or GRTLSFNTYAMG (SEQ ID NO: 163); (iii) SSYRMG (SEQ ID NO: 164), SINVMG (SEQ ID NO: 165), SSNAMG (SEQ ID NO: 166), SSYAMG (SEQ ID NO: 167), SSDAMG (SEQ ID NO: 168), SINVMG (SEQ ID NO: 169), STYRMG (SEQ ID NO: 170), TRYAMG (SEQ ID NO: 171), TTYRMG (SEQ ID NO: 172), or SFNTYAMG (SEQ ID NO: 173); or (iv) GLTFSSYRMG (SEQ ID NO: 174), GSIFSINVMG (SEQ ID NO: 175), GTSVSSNAMG (SEQ ID NO: 176), GRTFSSYAMG (SEQ ID NO: 177), GSSVSSDAMG (SEQ ID NO: 178), RSIGSINVMG (SEQ ID NO: 179), GRTFSTYRMG (SEQ ID NO: 180), GFTFTRYAMG (SEQ ID NO: 181), GRTFTTYRMG (SEQ ID NO: 182), or GRTLSFNTYAMG (SEQ ID NO: 183).

86. The pIgR modulator of any one of embodiments 71 to 85, wherein the VHH domain comprises a complementarity determining region 2 (CDR2) comprising the amino acid sequence of AIDWNGRGTYYRYYADSVKG (SEQ ID NO: 30), RINGGGITHYAESVKG (SEQ ID NO: 31), FIDRIATTTIATSVKG (SEQ ID NO: 32), AITWNGGTTYYADSVKG (SEQ ID NO: 33), FISGGGTTTYADSVKG (SEQ ID NO: 34), RITGGGSTHYAESVKG (SEQ ID NO: 35), AISWSGGSTTYADPVKG (SEQ ID NO: 36), AISWSGSSAGYGDSVKG (SEQ ID NO: 37), AIRWSGGRTLYADSVKG (SEQ ID NO: 38), or SITWNGGSTSYADSVKG (SEQ ID NO: 39).

87. The pIgR modulator of any one of embodiments 71 to 85, wherein the VHH domain comprises a complementarity determining region 2 (CDR2) comprising the amino acid sequence of DWNGRGTYY (SEQ ID NO: 40), WNGRGTY (SEQ ID NO: 260), NGGGI (SEQ ID NO: 41), GGG (SEQ ID NO: 261), DRIAT (SEQ ID NO: 42), RIA (SEQ ID NO: 262), TWNGGT (SEQ ID NO: 43), WNGG (SEQ ID NO: 263), SGGGT (SEQ ID NO: 44), GGG (SEQ ID NO: 264), TGGGS (SEQ ID NO: 45), GGG (SEQ ID NO: 265), SWSGGS (SEQ ID NO: 46), WSGG (SEQ ID NO: 266), SWSGSS (SEQ ID NO: 47), WSGS (SEQ ID NO: 267), RWSGGR (SEQ ID NO: 48), WSGG (SEQ ID NO: 268), TWNGGS (SEQ ID NO: 49) or WNGG (SEQ ID NO: 269).

88. The pIgR modulator of any one of embodiments 71 to 85, wherein the VHH domain comprises a complementarity determining region 2 (CDR2) comprising the amino acid sequence of (i) IDWNGRGTYY (SEQ ID NO: 50), IDWNGRGTYYR (SEQ ID NO: 270), INGGGIT (SEQ ID NO: 51), IDRIATT (SEQ ID NO: 52), ITWNGGTT (SEQ ID NO: 53), ISGGGTT (SEQ ID NO: 54), ITGGGST (SEQ ID NO: 55), ISWSGGST (SEQ ID NO: 56), ISWSGSSA (SEQ ID NO: 57), IRWSGGRT (SEQ ID NO: 58), or ITWNGGST (SEQ ID NO: 59); (ii) AIDWNGRGTYYRYYADSVKG (SEQ ID NO: 184), RINGGGITHYAESVKG (SEQ ID NO: 185), FIDRIATTTIATSVKG (SEQ ID NO: 186), AITWNGGTTYYADSVKG (SEQ ID NO: 187), FISGGGTTTYADSVKG (SEQ ID NO: 188), RITGGGSTHYAESVKG (SEQ ID NO: 189), AISWSGGSTTYADPVKG (SEQ ID NO: 190), AISWSGSSAGYGDSVKG (SEQ ID NO: 191), AIRWSGGRTLYADSVKG (SEQ ID NO: 192), or SITWNGGSTSYADSVKG (SEQ ID NO: 193); (iii) FVAAIDWNGRGTYYRY (SEQ ID NO: 194), LVARINGGGITH (SEQ ID NO: 195), WVGFIDRIATTT (SEQ ID NO: 196), FVAAITWNGGTTY (SEQ ID NO: 197), WVAFISGGGTTT (SEQ ID NO: 198), LVARITGGGSTH (SEQ ID NO: 199), FVAAISWSGGSTT (SEQ ID NO: 200), FVAAISWSGSSAG (SEQ ID NO: 201), FVAAIRWSGGRTL (SEQ ID NO: 202), or FVASITWNGGSTS (SEQ ID NO: 203); or (iv) AIDWNGRGTYYRY (SEQ ID NO: 204), RINGGGITH (SEQ ID NO: 205), FIDRIATTT (SEQ ID NO: 206), AITWNGGTTY (SEQ ID NO: 207), FISGGGTTT (SEQ ID NO: 208), RITGGGSTH (SEQ ID NO: 209), AISWSGGSTT (SEQ ID NO: 210), AISWSGSSAG (SEQ ID NO: 211), AIRWSGGRTL (SEQ ID NO: 212), or SITWNGGSTS (SEQ ID NO: 213).

89. The pIgR modulator of any one of embodiments 71 to 88, wherein the VHH domain comprises a complementarity determining region 3 (CDR3) comprising the amino acid sequence of GSIDLNWYGGMDY (SEQ ID NO: 60), TTVLTDPRVLNEYAT (SEQ ID NO: 61), DVFGSSGYVETY (SEQ ID NO: 62), PLTAR (SEQ ID NO: 63), DPFNQGY (SEQ ID NO: 64), PLTSR (SEQ ID NO: 65), MVNPIITAWGTIGVREIPDYDY (SEQ ID NO: 66), DQRGY (SEQ ID NO: 67), QRGY (SEQ ID NO: 271), DPFNQGY (SEQ ID NO: 68), DLAEYSGTYSSPADSPAGYDY (SEQ ID NO: 69), or ARYYVSGTYFPANY (SEQ ID NO: 70).

90. The pIgR modulator of any one of embodiments 71 to 88, wherein the VHH domain comprises a complementarity determining region 3 (CDR3) comprising the amino acid sequence of GSIDLNWYGGMDY (SEQ ID NO: 71), SIDLNWYGGMD (SEQ ID NO: 272), TTVLTDPRVLNEYAT (SEQ ID NO: 72), TVLTDPRVLNEYA (SEQ ID NO: 273), DVFGSSGYVETY (SEQ ID NO: 73), VFGSSGYVET (SEQ ID NO: 274), PLTAR (SEQ ID NO: 74), LTA (SEQ ID NO: 275), DPFNQGY (SEQ ID NO: 75), PFNQG (SEQ ID NO: 276), PLTSR (SEQ ID NO: 76), LTS (SEQ ID NO: 277), MVNPIITAWGTIGVREIPDYDY (SEQ ID NO: 77), VNPIITAWGTIGVREIPDYD (SEQ ID NO: 278), DQRGY (SEQ ID NO: 78), RG (SEQ ID NO: 279), DPFNQGY (SEQ ID NO: 79), PFNQG (SEQ ID NO: 280), DLAEYSGTYSSPADSPAGYDY (SEQ ID NO: 80), LAEYSGTYSSPADSPAGYD (SEQ ID NO: 281), ARYYVSGTYFPANY (SEQ ID NO: 81), or RYYVSGTYFPAN (SEQ ID NO: 282).

91. The pIgR modulator of any one of embodiments 71 to 88, wherein the VHH domain comprises a complementarity determining region 3 (CDR3) comprising the amino acid sequence of (i) CAAGSIDLNWYGGMDY (SEQ ID NO: 82), AAGSIDLNWYGGMDY (SEQ ID NO: 283), CAATTVLTDPRVLNEYAT (SEQ ID NO: 83), AATTVLTDPRVLNEYAT (SEQ ID NO: 284), KADVFGSSGYVETY (SEQ ID NO: 84), NHPLTAR (SEQ ID NO: 85), AADPFNQGY (SEQ ID NO: 86), NHPLTSR (SEQ ID NO: 87), ASMVNPIITAWGTIGVREIPDYDY (SEQ ID NO: 88), NDQRGY (SEQ ID NO: 89), AADPFNQGY (SEQ ID NO: 90), AADLAEYSGTYSSPADSPAGYDY (SEQ ID NO: 91), or AAARYYVSGTYFPANY (SEQ ID NO: 92); (ii) GSIDLNWYGGMDY (SEQ ID NO: 214), TTVLTDPRVLNEYAT (SEQ ID NO: 215), DVFGSSGYVETY (SEQ ID NO: 216), PLTAR (SEQ ID NO: 217), DPFNQGY (SEQ ID NO: 218), PLTSR (SEQ ID NO: 219), MVNPIITAWGTIGVREIPDYDY (SEQ ID NO: 220), QRGY (SEQ ID NO: 221), DPFNQGY (SEQ ID NO: 222), DLAEYSGTYSSPADSPAGYDY (SEQ ID NO: 223), or ARYYVSGTYFPANY (SEQ ID NO: 224); (iii) AAGSIDLNWYGGMD (SEQ ID NO: 225), AATTVLTDPRVLNEYA (SEQ ID NO: 226), KADVFGSSGYVET (SEQ ID NO: 227), NHPLTA (SEQ ID NO: 228), AADPFNQG (SEQ ID NO: 229), NHPLTS (SEQ ID NO: 230), ASMVNPIITAWGTIGVREIPDYD (SEQ ID NO: 231), NDQRG (SEQ ID NO: 232), AADPFNQG (SEQ ID NO: 233), AADLAEYSGTYSSPADSPAGYD (SEQ ID NO: 234), or AAARYYVSGTYFPAN (SEQ ID NO: 235); or (iv) GSIDLNWYGGMDY (SEQ ID NO: 236), TTVLTDPRVLNEYAT (SEQ ID NO: 237), DVFGSSGYVETY (SEQ ID NO: 238), PLTAR (SEQ ID NO: 239), DPFNQGY (SEQ ID NO: 240), PLTSR (SEQ ID NO: 241), MVNPIITAWGTIGVREIPDYDY (SEQ ID NO: 242), QRGY (SEQ ID NO: 243), DPFNQGY (SEQ ID NO: 244), DLAEYSGTYSSPADSPAGYDY (SEQ ID NO: 245), or ARYYVSGTYFPANY (SEQ ID NO: 246), including wherein optionally the VHH domain comprises a CDR1 sequence, a CDR2 sequence, and a CDR3 sequence of the VHH domain selected from the group consisting of:
a) VHH1:
   i) the CDR1 sequence of SYRMG (SEQ ID NO: 1), the CDR2 sequence of AIDWNGRGTYYRYYADSVKG (SEQ ID NO: 30), and the CDR3 sequence of GSIDLNWYGGMDY (SEQ ID NO: 60);
   ii) the CDR1 sequence of GLTFSSY (SEQ ID NO: 10), the CDR2 sequence of DWNGRGTYY (SEQ ID NO: 40) or WNGRGTY (SEQ ID NO: 260), and the CDR3 sequence of GSIDLNWYGGMDY (SEQ ID NO: 71) or SIDLNWYGGMD (SEQ ID NO: 272);
   iii) the CDR1 sequence of GLTFSSYR (SEQ ID NO: 20), the CDR2 sequence of IDWNGRGTYY (SEQ ID NO: 50) or IDWNGRGTYYR (SEQ ID NO: 270), and the CDR3 sequence of CAAGSIDLNWYGGMDY (SEQ ID NO: 82) or AAGSIDLNWYGGMDY (SEQ ID NO: 283);
   iv) the CDR1 sequence of GLTFSSYRMG (SEQ ID NO: 154), the CDR 2 sequence of AIDWNGRGTYYRYYADSVKG (SEQ ID NO: 184), and the CDR3 sequence of GSIDLNWYGGMDY (SEQ ID NO: 214);
   v) the CDR1 sequence of SSYRMG (SEQ ID NO: 164), the CDR2 sequence of FVAAIDWNGRGTYYRY (SEQ ID NO: 194), and the CDR3 sequence of AAGSIDLNWYGGMD (SEQ ID NO: 225); or
   vi) the CDR1 sequence of GLTFSSYRMG (SEQ ID NO: 174), the CDR2 sequence of AIDWNGRGTYYRY (SEQ ID NO: 204), and the CDR3 sequence of GSIDLNWYGGMDY (SEQ ID NO: 236);
b) VHH2:
   i) the CDR1 sequence of SYRMG (SEQ ID NO: 1), the CDR2 sequence of AIDWNGRGTYYRYYADSVKG (SEQ ID NO: 30), and the CDR3 sequence of TTVLTDPRVLNEYAT (SEQ ID NO: 61);
   ii) the CDR1 sequence of GLTFSSY (SEQ ID NO: 10), the CDR2 sequence of DWNGRGTYY (SEQ ID NO: 40) or WNGRGTY (SEQ ID NO: 260), and the CDR3 sequence of TTVLTDPRVLNEYAT (SEQ ID NO: 72) or TVLTDPRVLNEYA (SEQ ID NO: 273);
   iii) the CDR1 sequence of GLTFSSYR (SEQ ID NO: 20), the CDR2 sequence of IDWNGRGTYY (SEQ ID NO: 50) or IDWNGRGTYYR (SEQ ID NO: 270), and the CDR3 sequence of CAATTVLTDPRVLNEYAT (SEQ ID NO: 83) or AATTVLTDPRVLNEYAT (SEQ ID NO: 284);
   iv) the CDR1 sequence of GLTFSSYRMG (SEQ ID NO: 154), the CDR2 sequence of AIDWNGRGTYYRYYADSVKG (SEQ ID NO: 184), and the CDR3 sequence of TTVLTDPRVLNEYAT (SEQ ID NO: 215);
   v) the CDR1 sequence of SSYRMG (SEQ ID NO: 164), the CDR2 sequence of FVAAIDWNGRGTYYRY (SEQ ID NO: 194), and the CDR3 sequence of AATTVLTDPRVLNEYA (SEQ ID NO: 226); or
   vi) the CDR1 sequence of GLTFSSYRMG (SEQ ID NO: 174), the CDR2 sequence of AIDWNGRGTYYRY (SEQ ID NO: 204), and the CDR3 sequence of TTVLTDPRVLNEYAT (SEQ ID NO: 237);
c) VHH3:
   i) the CDR1 sequence of INVMG (SEQ ID NO: 2), the CDR2 sequence of RINGGGITHYAESVKG (SEQ ID NO: 31), and the CDR3 sequence of DVFGSSGYVETY (SEQ ID NO: 62);
   ii) the CDR1 sequence of GSIFSIN (SEQ ID NO: 11), the CDR2 sequence of NGGGI (SEQ ID NO: 41) or GGG (SEQ ID NO: 261), and the CDR3 sequence of DVFGSSGYVETY (SEQ ID NO: 73) or VFGSSGYVET (SEQ ID NO: 274);
   iii) the CDR1 sequence of GSIFSINV (SEQ ID NO: 21), the CDR2 sequence of INGGGIT (SEQ ID NO: 51), and the CDR3 sequence of KADVFGSSGYVETY (SEQ ID NO: 84);
   iv) the CDR1 sequence of GSIFSINVMG (SEQ ID NO: 155), the CDR2 sequence of RINGGGITHYAESVKG (SEQ ID NO: 185), and the CDR3 sequence of DVFGSSGYVETY (SEQ ID NO: 216);
   v) the CDR1 sequence of SINVMG (SEQ ID NO: 165), the CDR2 sequence of LVARINGGGITH (SEQ ID NO: 195), and the CDR3 sequence of KADVFGSSGYVET (SEQ ID NO: 227); or
   vi) the CDR1 sequence of GSIFSINVMG (SEQ ID NO: 175), the CDR2 sequence of RINGGGITH (SEQ ID NO: 205), and the CDR3 sequence of DVFGSSGYVETY (SEQ ID NO: 238);
d) VHH4:
   i) the CDR1 sequence of SNAMG (SEQ ID NO: 3), the CDR2 sequence of FIDRIATTTIATSVKG (SEQ ID NO: 32), and the CDR3 sequence of PLTAR (SEQ ID NO: 63);
   ii) the CDR1 sequence of GTSVSSN (SEQ ID NO: 12), the CDR2 sequence of DRIAT (SEQ ID NO: 42) or RIA (SEQ ID NO: 262), and the CDR3 sequence of PLTAR (SEQ ID NO: 74) or LTA (SEQ ID NO: 275);
   iii) the CDR1 sequence of GTSVSSNA (SEQ ID NO: 22), the CDR2 sequence of IDRIATT (SEQ ID NO: 52), and the CDR3 sequence of NHPLTAR (SEQ ID NO: 85);
   iv) the CDR1 sequence of GTSVSSNAMG (SEQ ID NO: 156), the CDR2 sequence of FIDRIATTTIATSVKG (SEQ ID NO: 186), and the CDR3 sequence of PLTAR (SEQ ID NO: 217);
   v) the CDR1 sequence of SSNAMG (SEQ ID NO: 166), the CDR2 sequence of WVGFIDRIATTT (SEQ ID NO: 196), and the CDR3 sequence of NHPLTA (SEQ ID NO: 228); or
   vi) the CDR1 sequence of GTSVSSNAMG (SEQ ID NO: 176), the CDR2 sequence of FIDRIATTT (SEQ ID NO: 206), and the CDR3 sequence of PLTAR (SEQ ID NO: 239);
e) VHH5:
   i) the CDR1 sequence of SYAMG (SEQ ID NO: 4), the CDR2 sequence of AITWNGGTTYYADSVKG (SEQ ID NO: 33), and the CDR3 sequence of DPFNQGY (SEQ ID NO: 64);
   ii) the CDR1 sequence of GRTFSSY (SEQ ID NO: 13), the CDR2 sequence of TWNGGT (SEQ ID NO: 43) or WNGG (SEQ ID NO: 263), and the CDR3 sequence of DPFNQGY (SEQ ID NO: 75) or PFNQG (SEQ ID NO: 276);
   iii) the CDR1 sequence of GRTFSSYA (SEQ ID NO: 23), the CDR2 sequence of ITWNGGTT (SEQ ID NO: 53), and the CDR3 sequence of AADPFNQGY (SEQ ID NO: 86);
   iv) the CDR1 sequence of GRTFSSYAMG (SEQ ID NO: 157), the CDR2 sequence of AITWNGGTTYYADSVKG (SEQ ID NO: 187), and the CDR3 sequence of DPFNQGY (SEQ ID NO: 218);
   v) the CDR1 sequence of SSYAMG (SEQ ID NO: 167), the CDR2 sequence of FVAAITWNGGTTY (SEQ ID NO: 197), and the CDR3 sequence of AADPFNQG (SEQ ID NO: 229); or
   vi) the CDR1 sequence of GRTFSSYAMG (SEQ ID NO: 177), the CDR2 sequence of AITWNGGTTY (SEQ ID NO: 207), and the CDR3 sequence of DPFNQGY (SEQ ID NO: 240);
f) VHH6:
   i) the CDR1 sequence of SDAMG (SEQ ID NO: 5), the CDR2 sequence of FISGGGTTTYADSVKG (SEQ ID NO: 34), and the CDR3 sequence of PLTSR (SEQ ID NO: 65);
   ii) the CDR1 sequence of GSSVSSD (SEQ ID NO: 14), the CDR2 sequence of SGGGT (SEQ ID NO: 44) or GGG (SEQ ID NO: 264), and the CDR3 sequence of PLTSR (SEQ ID NO: 76) or LTS (SEQ ID NO: 277);
   iii) the CDR1 sequence of GSSVSSDA (SEQ ID NO: 24), the CDR2 sequence of ISGGGTT (SEQ ID NO: 54), and the CDR3 sequence of NHPLTSR (SEQ ID NO: 87);
   iv) the CDR1 sequence of GSSVSSDAMG (SEQ ID NO: 158), the CDR2 sequence of FISGGGTTTYADSVKG (SEQ ID NO: 188), and the CDR3 sequence of PLTSR (SEQ ID NO: 219);
   v) the CDR1 sequence of SSDAMG (SEQ ID NO: 168), the CDR2 sequence of WVAFISGGGTTT (SEQ ID NO: 198), and the CDR3 sequence of NHPLTS (SEQ ID NO: 230); or
   vi) the CDR1 sequence of GSSVSSDAMG (SEQ ID NO: 178), the CDR2 sequence of FISGGGTTT (SEQ ID NO: 208), and the CDR3 sequence of PLTSR (SEQ ID NO: 241);
g) VHH7:
   i) the CDR1 sequence of INVMG (SEQ ID NO: 6), the CDR2 sequence of RITGGGSTHYAESVKG (SEQ ID NO: 35), and the CDR3 sequence of MVNPIITAWGTIGVREIPDYDY (SEQ ID NO: 66);
   ii) the CDR1 sequence of RSIGSIN (SEQ ID NO: 15), the CDR2 sequence of TGGGS (SEQ ID NO: 45) or GGG (SEQ ID NO: 265), and the CDR3 sequence of MVNPIITAWGTIGVREIPDYDY (SEQ ID NO: 77) or VNPIITAWGTIGVREIPDYD (SEQ ID NO: 278);
   iii) the CDR1 sequence of RSIGSINV (SEQ ID NO: 25), the CDR2 sequence of ITGGGST (SEQ ID NO: 55), and the CDR3 sequence of ASMVNPIITAWGTIGVREIPDYDY (SEQ ID NO: 88);
   iv) the CDR1 sequence of RSIGSINVMG (SEQ ID NO: 159), the CDR2 sequence of RITGGGSTHYAESVKG (SEQ ID NO: 189), and the CDR3 sequence of MVNPIITAWGTIGVREIPDYDY (SEQ ID NO: 220);
   v) the CDR1 sequence of SINVMG (SEQ ID NO: 169), the CDR2 sequence of LVARITGGGSTH (SEQ ID NO: 199), and the CDR3 sequence of ASMVNPIITAWGTIGVREIPDYD (SEQ ID NO: 231); or
   vi) the CDR1 sequence of RSIGSINVMG (SEQ ID NO: 179), the CDR2 sequence of RITGGGSTH (SEQ ID NO: 209), and the CDR3 sequence of MVNPIITAWGTIGVREIPDYDY (SEQ ID NO: 242);
h) VHH9:
   i) the CDR1 sequence of TYRMG (SEQ ID NO: 7), the CDR2 sequence of AISWSGGSTTYADPVKG (SEQ ID NO: 36), and the CDR3 sequence of DQRGY (SEQ ID NO: 67) or QRGY (SEQ ID NO: 271);
   ii) the CDR1 sequence of GRTFSTY (SEQ ID NO: 16), the CDR2 sequence of SWSGGS (SEQ ID NO: 46) or WSGG (SEQ ID NO: 266), and the CDR3 sequence of DQRGY (SEQ ID NO: 78) or RG (SEQ ID NO: 279);
   iii) the CDR1 sequence of GRTFSTYR (SEQ ID NO: 26), the CDR2 sequence of ISWSGGST (SEQ ID NO: 56), and the CDR3 sequence of NDQRGY (SEQ ID NO: 89);
   iv) the CDR1 sequence of GRTFSTYRMG (SEQ ID NO: 160), the CDR2 sequence of AISWSGGSTTYADPVKG (SEQ ID NO: 190), and the CDR3 sequence of QRGY (SEQ ID NO: 221);
   v) the CDR1 sequence of STYRMG (SEQ ID NO: 170), the CDR2 sequence of FVAAISWSGGSTT (SEQ ID NO: 200), and the CDR3 sequence of NDQRG (SEQ ID NO: 232); or
   vi) the CDR1 sequence of GRTFSTYRMG (SEQ ID NO: 180), the CDR2 sequence of AISWSGGSTT (SEQ ID NO: 210), and the CDR3 sequence of QRGY (SEQ ID NO: 243);
i) VHH10:
   i) the CDR1 sequence of RYAMG (SEQ ID NO: 8), the CDR2 sequence of AISWSGSSAGYGDSVKG (SEQ ID NO: 37), and the CDR3 sequence of DPFNQGY (SEQ ID NO: 68);
   ii) the CDR1 sequence of GFTFTRY (SEQ ID NO: 17), the CDR2 sequence of SWSGSS (SEQ ID NO: 47) or WSGS (SEQ ID NO: 267), and the CDR3 sequence of DPFNQGY (SEQ ID NO: 79) or PFNQG (SEQ ID NO: 280);
   iii) the CDR1 sequence of GFTFTRYA (SEQ ID NO: 27), the CDR2 sequence of ISWSGSSA (SEQ ID NO: 57), and the CDR3 sequence of AADPFNQGY (SEQ ID NO: 90);
   iv) the CDR1 sequence of GFTFTRYAMG (SEQ ID NO: 161), the CDR2 sequence of AISWSGSSAGYGDSVKG (SEQ ID NO: 191), and the CDR3 sequence of DPFNQGY (SEQ ID NO: 222);
   v) the CDR1 sequence of TRYAMG (SEQ ID NO: 171), the CDR2 sequence of FVAAISWSGSSAG (SEQ ID NO: 201), and the CDR3 sequence of AADPFNQG (SEQ ID NO: 233); or
   vi) the CDR1 sequence of GFTFTRYAMG (SEQ ID NO: 181), the CDR2 sequence of AISWSGSSAG (SEQ ID NO: 211), and the CDR3 sequence of DPFNQGY (SEQ ID NO: 244);
j) VHH11:
   i) the CDR1 sequence of FTTYRMG (SEQ ID NO: 258) or TYRMG (SEQ ID NO: 259), the CDR2 sequence of AIRWSGGRTLYADSVKG (SEQ ID NO: 38), and the CDR3 sequence of DLAEYSGTYSSPADSPAGYDY (SEQ ID NO: 69);
   ii) the CDR1 sequence of GRTFTTY (SEQ ID NO: 18), the CDR2 sequence of RWSGGR (SEQ ID NO: 48) or WSGG (SEQ ID NO: 268), and the CDR3 sequence of DLAEYSGTYSSPADSPAGYDY (SEQ ID NO: 80) or LAEYSGTYSSPADSPAGYD (SEQ ID NO: 281);
   iii) the CDR1 sequence of GRTFTTYR (SEQ ID NO: 28), the CDR2 sequence of IRWSGGRT (SEQ ID NO: 58), and the CDR3 sequence of AADLAEYSGTYSSPADSPAGYDY (SEQ ID NO: 91);
   iv) the CDR1 sequence of GRTFTTYRMG (SEQ ID NO: 162), the CDR2 sequence of AIRWSGGRTLYADSVKG (SEQ ID NO: 192), and the CDR3 sequence of DLAEYSGTYSSPADSPAGYDY (SEQ ID NO: 223);
   v) the CDR1 sequence of TTYRMG (SEQ ID NO: 172), the CDR2 sequence of FVAAIRWSGGRTL (SEQ ID NO: 202), and the CDR3 sequence of AADLAEYSGTYSSPADSPAGYD (SEQ ID NO: 234); or
   vi) the CDR1 sequence of GRTFTTYRMG (SEQ ID NO: 182), the CDR2 sequence of AIRWSGGRTL (SEQ ID NO: 212), and the CDR3 sequence of DLAEYSGTYSSPADSPAGYDY (SEQ ID NO: 245); and
k) VHH12:
   i) the CDR1 sequence of FNTYAMG (SEQ ID NO: 9), the CDR2 sequence of SITWNGGSTSYADSVKG (SEQ ID NO: 39), and the CDR3 sequence of ARYYVSGTYFPANY (SEQ ID NO: 70);
   ii) the CDR1 sequence of GRTLSFNTY (SEQ ID NO: 19), the CDR2 sequence of TWNGGS (SEQ ID NO: 49) or WNGG (SEQ ID NO: 269), and the CDR3 sequence of ARYYVSGTYFPANY (SEQ ID NO: 81) or RYYVSGTYFPAN (SEQ ID NO: 282);
   iii) the CDR1 sequence of GRTLSFNTYA (SEQ ID NO: 29), the CDR2 sequence of ITWNGGST (SEQ ID NO: 59), and the CDR3 sequence of AAARYYVSGTYFPANY (SEQ ID NO: 92);
   iv) the CDR1 sequence of GRTLSFNTYAMG (SEQ ID NO: 163), the CDR2 sequence of SITWNGGSTSYADSVKG (SEQ ID NO: 193), and the CDR3 sequence of ARYYVSGTYFPANY (SEQ ID NO: 224);
   v) the CDR1 sequence of SFNTYAMG (SEQ ID NO: 173), the CDR2 sequence of FVASITWNGGSTS (SEQ ID NO: 203), and the CDR3 sequence of AAARYYVSGTYFPAN (SEQ ID NO: 235); or
   vi) the CDR1 sequence of GRTLSFNTYAMG (SEQ ID NO: 183), the CDR2 sequence of SITWNGGSTS (SEQ ID NO: 213), and the CDR3 sequence of ARYYVSGTYFPANY (SEQ ID NO: 246).

92. The pIgR modulator of any one of embodiments 71 to 82, wherein the VHH domain is comprised of a germline sequence of

(SEQ ID NO: 285)
QVQLVESGGGLVQAGGSLKLACAAPGLTFSSYRMGWFRQAPGQEREFVAAI
DWNGRGTYYRYYADSVKGRSTISRDNAKNTMYLQMNSLKPEDTAVYYCA,
(SEQ ID NO: 286)
EVQVVESGGGLVQAGGSLKLACAAPGLTFSSYRMGWFRQAPGQEREFVAAI
DWNGRGTYYRYYADSVKGRSTISRDNAKNTVYLQMNSLKPEDTAVYYCA,
(SEQ ID NO: 287)
QLQLVESGGGLVQPGGSLRLSCAASGSIFSINVMGWYRQAPGKQRELVARI
NGGGITHYAESVKGRFTISRDNAKNTVYLQMNSLKPEDTAAYYCKA,
(SEQ ID NO: 288)
EVQVVESGGGLVQAGGSLRLSCAVSGTSVSSNAMGWYRQAPGKQREWVGFI
DRIATTTIATSVKGRFAITRDNAKNTVYLQMSGLKPEDTAVYYCN,
(SEQ ID NO: 289)
QVQLVESGGGLVQAGGSLRLSCAASGRTFSSYAMGWFRQAPGKEREFVAAI
TWNGGTTYYADSVKGRFTISRDNAKNTVYLQMNSLKPEDTAVYYCAA,
(SEQ ID NO: 290)
EVQLVESGGGLVQAGGSLRLSCAVSGSSVSSDAMGWYRQAPGNQRAWVAFI
SGGGTTTYADSVKGRFTISRDNTKNTVYLHMNSLKPEDTAVYYCN,
(SEQ ID NO: 291)
EVQVVESGGGLVQAGGSLRLACVASRSIGSINVMGWYRQAPGKQRDLVARI
TGGGSTHYAESVKGRFTISRDNAKNTVYLQMNSLEPEDTAVYYC,
(SEQ ID NO: 292)
QVQLVESGGGLVQAGGSLRLSCAVSGRTFSTYRMGWFRQAPGKERSFVAAI
SWSGGSTTYADPVKGRFTISRDNAKNTVYLRMNSLKPEDTAVYYCN,
(SEQ ID NO: 293)
EVQVVESGGGLVQAGGSLRLSCAASGFTFTRYAMGWFRQAPGKERSFVAAI
SWSGSSAGYGDSVKGRFTISRDNAKNTLYLQMNSLKPEDTAVYYCA,
(SEQ ID NO: 294)
EVQVVESGGGLVQAGGSLRLSCAASGRTFTTYRMGWFRQAPGKEREFVAAI
RWSGGRTLYADSVKGRFTISRDNAKNTAYLQMNNLRPEDTAVYYCAA,
or
(SEQ ID NO: 295)
QVQLVETGGGLVQAGDSLRLSCAASGRTLSFNTYAMGWFRQAPGKEREFVA
SITWNGGSTSYADSVKGRFTITRDNAKNTATLRMNSLQPDDTAVYYCAA.

93. The pIgR modulator of any one of embodiments 71 to 82, wherein the VHH domain is comprised of a J segment, wherein the J segment comprises the sequence of DYWGQGTQVTVSS (SEQ ID NO: 296), EYATWGQGTQVTVSS (SEQ ID NO: 297), YWGQGTQVTVSS (SEQ ID NO: 298), WGQGTQVTVSS (SEQ ID NO: 299), DYDYWGQGTQVTVSS (SEQ ID NO: 300), WGQGTLVTVSS (SEQ ID NO: 301), YDYWGQGTQVTVSS (SEQ ID NO: 302), or NYWGQGTQVTVSS (SEQ ID NO: 303).

94. The pIgR modulator of any one of embodiments 71 to 82, wherein the VHH domain is comprised of a sequence of

(SEQ ID NO: 93)
QVQLVESGGGLVQAGGSLKLACAAPGLTFSSYRMGWFRQAPGQEREFVA
AIDWNGRGTYYRYYADSVKGRSTISRDNAKNTMYLQMNSLKPEDTAVYY
CAAGSIDLNWYGGMDYWGQGTQVTVSS,
(SEQ ID NO: 94)
EVQVVESGGGLVQAGGSLKLACAAPGLTFSSYRMGWFRQAPGQEREFVA
AIDWNGRGTYYRYYADSVKGRSTISRDNAKNTVYLQMNSLKPEDTAVYY
CAATTVLTDPRVLNEYATWGQGTQVTVSS,
(SEQ ID NO: 95)
EVQVVESGGGLVQAGGSLRLSCAVSGTSVSSNAMGWYRQAPGKQREWVG
FIDRIATTTIATSVKGRFAITRDNAKNTVYLQMSGLKPEDTAVYYCNHP
LTARWGQGTQVTVSS,
(SEQ ID NO: 96)
QVQLVESGGGLVQAGGSLRLSCAASGRTFSSYAMGWFRQAPGKEREFVA
AITWNGGTTYYADSVKGRFTISRDNAKNTVYLQMNSLKPEDTAVYYCAA
DPFNQGYWGQGTQVTVSS,
(SEQ ID NO: 97)
EVQLVESGGGLVQAGGSLRLSCAVSGSSVSSDAMGWYRQAPGNQRAWVA
FISGGGTTTYADSVKGRFTISRDNTKNTVYLHMNSLKPEDTAVYYCNHP
LTSRWGQGTQVTVSS,
(SEQ ID NO: 98)
EVQVVESGGGLVQAGGSLRLACVASRSIGSINVMGWYRQAPGKQRDLVA
RITGGGSTHYAESVKGRFTISRDNAKNTVYLQMNSLEPEDTAVYYCASM
VNPIITAWGTIGVREIPDYDYWGQGTQVTVSS,
(SEQ ID NO: 99)
QVQLVESGGGLVQAGGSLRLSCAVSGRTFSTYRMGWFRQAPGKERSFVA
AISWSGGSTTYADPVKGRFTISRDNAKNTVYLRMNSLKPEDTAVYYCND
QRGYWGQGTLVTVSS,
(SEQ ID NO: 100)
EVQVVESGGGLVQAGGSLRLSCAASGFTFTRYAMGWFRQAPGKERSFVA
AISWSGSSAGYGDSVKGRFTISRDNAKNTLYLQMNSLKPEDTAVYYCAA
DPFNQGYWGQGTQVTVSS,
(SEQ ID NO: 101)
EVQVVESGGGLVQAGGSLRLSCAASGRTFTTYRMGWFRQAPGKEREFVA
AIRWSGGRTLYADSVKGRFTISRDNAKNTAYLQMNNLRPEDTAVYYCAA
DLAEYSGTYSSPADSPAGYDYWGQGTQVTVSS,
or
(SEQ ID NO: 102)
QVQLVETGGGLVQAGDSLRLSCAASGRTLSFNTYAMGWFRQAPGKEREF
VASITWNGGSTSYADSVKGRFTITRDNAKNTATLRMNSLQPDDTAVYYC
AAARYYVSGTYFPANYWGQGTQVTVSS.

95. The pIgR modulator of any one of embodiments 71 to 82, wherein the VHH domain comprises a complementarity determining region 1 (CDR1) comprising the amino acid sequence of SYRMG (SEQ ID NO: 1), GLTFSSY (SEQ ID NO: 10), or GLTFSSYR (SEQ ID NO: 20).

96. The pIgR modulator of embodiment 95, wherein the CDR1 comprises the amino acid sequence of SYRMG (SEQ ID NO: 1).

97. The pIgR modulator of embodiment 95, wherein the CDR1 comprises the amino acid sequence of GLTFSSY (SEQ ID NO: 10).

98. The pIgR modulator of embodiment 95, wherein the CDR1 comprises the amino acid sequence of GLTFSSYR (SEQ ID NO: 20).

99. The pIgR modulator of any one of embodiments 71 to 82, or embodiments 95 to 98, wherein the VHH domain comprises a complementarity determining region 2 (CDR2) comprising the amino acid sequence of AIDWNGRGTYYRYYADSVKG (SEQ ID NO: 30), DWNGRGTYY (SEQ ID NO: 40), or IDWNGRGTYY (SEQ ID NO: 50).

100. The pIgR modulator of embodiment 99, wherein the CDR2 comprises the amino acid sequence of AIDWNGRGTYYRYYADSVKG (SEQ ID NO: 30).

101. The pIgR modulator of embodiment 99, wherein the CDR2 comprises the amino acid sequence of DWNGRGTYY (SEQ ID NO: 40).

102. The pIgR modulator of embodiment 99, wherein the CDR2 comprises the amino acid sequence of IDWNGRGTYY (SEQ ID NO: 50).

103. The pIgR modulator of any one of embodiments 71 to 82, or embodiments 95 to 98, wherein the VHH domain comprises a complementarity determining region 3 (CDR3) comprising the amino acid sequence of TTVLTDPRVLNEYAT (SEQ ID NO: 61) or CAATTVLTDPRVLNEYAT (SEQ ID NO: 83).

104. The pIgR modulator of embodiment 103, wherein the CDR3 comprises the amino acid sequence of TTVLTDPRVLNEYAT (SEQ ID NO: 61).

105. The pIgR modulator of embodiment 103, wherein the CDR3 comprises the amino acid sequence of CAATTVLTDPRVLNEYAT (SEQ ID NO: 83).

106. The pIgR modulator of any one of embodiments 71 to 82, wherein the VHH domain is comprised of a germline sequence of

(SEQ ID NO: 286)
EVQVVESGGGLVQAGGSLKLACAAPGLTFSSYRMGWFRQAPGQEREFVAAI
DWNGRGTYYRYYADSVKGRSTISRDNAKNTVYLQMNSLKPEDTAVYYCA.

107. The pIgR modulator of any one of embodiments 71 to 82, or embodiments 95 to 106, wherein the VHH domain is comprised of a J segment, wherein the J segment comprises the sequence of EYATWGQGTQVTVSS (SEQ ID NO: 297).

108. The pIgR modulator of any one of embodiments 71 to 82, wherein the VHH domain is comprised of a sequence of

(SEQ ID NO: 94)
EVQVVESGGGLVQAGGSLKLACAAPGLTFSSYRMGWFRQAPGQEREFVA
AIDWNGRGTYYRYYADSVKGRSTISRDNAKNTVYLQMNSLKPEDTAVYY
CAATTVLTDPRVLNEYATWGQGTQVTVSS.

109. The pIgR modulator of any one of embodiments 71 to 82, wherein the VHH domain comprises a complementarity determining region 1 (CDR1) comprising the amino acid sequence of SDAMG (SEQ ID NO: 5), GSSVSSD (SEQ ID NO: 14), or GSSVSSDA (SEQ ID NO: 24).

110. The pIgR modulator of embodiment 109, wherein the CDR1 comprises the amino acid sequence of SDAMG (SEQ ID NO: 5).

111. The pIgR modulator of embodiment 109, wherein the CDR1 comprises the amino acid sequence of GSSVSSD (SEQ ID NO: 14).

112. The pIgR modulator of embodiment 109, wherein the CDR1 comprises the amino acid sequence of GSSVSSDA (SEQ ID NO: 24).

113. The pIgR modulator of any one of embodiments 71 to 82, or embodiments 109 to 112, wherein the VHH domain comprises a complementarity determining region 2 (CDR2) comprising the amino acid sequence of FISGGGTTTYADSVKG (SEQ ID NO: 34), SGGGT (SEQ ID NO: 44), or ISGGGTT (SEQ ID NO: 54).

114. The pIgR modulator of embodiment 113, wherein the CDR2 comprises the amino acid sequence of FISGGGTTTYADSVKG (SEQ ID NO: 34).

115. The pIgR modulator of embodiment 113, wherein the CDR2 comprises the amino acid sequence of SGGGT (SEQ ID NO: 44).

116. The pIgR modulator of embodiment 113, wherein the CDR2 comprises the amino acid sequence of ISGGGTT (SEQ ID NO: 54).

117. The pIgR modulator of any one of embodiments 71 to 82, or embodiments 109 to 116, wherein the VHH domain comprises a complementarity determining region 3 (CDR3) comprising the amino acid sequence of PLTSR (SEQ ID NO: 65) or NHPLTSR (SEQ ID NO: 87).

118. The pIgR modulator of embodiment 117, wherein the CDR3 comprises the amino acid sequence of PLTSR (SEQ ID NO: 65).

119. The pIgR modulator of embodiment 117, wherein the CDR3 comprises the amino acid sequence of NHPLTSR (SEQ ID NO: 87).

120. The pIgR modulator of any one of embodiments 71 to 82, wherein the VHH domain is comprised of a germline sequence of

(SEQ ID NO: 290)
EVQLVESGGGLVQAGGSLRLSCAVSGSSVSSDAMGWYRQAPGNQRAWVAF
ISGGGTTTYADSVKGRFTISRDNTKNTVYLHMNSLKPEDTAVYYCN.

121. The pIgR modulator of any one of embodiments 71 to 82, or embodiments 109 to 120, wherein the VHH domain is comprised of a J segment, wherein the J segment comprises the sequence of WGQGTQVTVSS (SEQ ID NO: 299).

122. The pIgR modulator of any one of embodiments 71 to 82, wherein the VHH domain is comprised of a sequence of

(SEQ ID NO: 98)
EVQLVESGGGLVQAGGSLRLSCAVSGSSVSSDAMGWYRQAPGNQRAWVAFI
SGGGTTTYADSVKGRFTISRDNTKNTVYLHMNSLKPEDTAVYYCNHPLTSR
WGQGTQVTVSS.

123. The pIgR modulator of any one of embodiments 71 to 82, wherein the VHH domain comprises a complementarity determining region 1 (CDR1) comprising the amino acid sequence of TYRMG (SEQ ID NO: 6), GRTFSTY (SEQ ID NO: 16), or GRTFSTYR (SEQ ID NO: 26).

124. The pIgR modulator of embodiment 123, wherein the CDR1 comprises the amino acid sequence of TYRMG (SEQ ID NO: 7).

125. The pIgR modulator of embodiment 123, wherein the CDR1 comprises the amino acid sequence of GRTFSTY (SEQ ID NO: 16).

126. The pIgR modulator of embodiment 123, wherein the CDR1 comprises the amino acid sequence of GRTFSTYR (SEQ ID NO: 26).

127. The pIgR modulator of any one of embodiments 71 to 82, or embodiments 123 to 126, wherein the VHH domain comprises a complementarity determining region 2 (CDR2) comprising the amino acid sequence of AISWSGGSTTYADPVKG (SEQ ID NO: 36), SWSGGS (SEQ ID NO: 46), or ISWSGGST (SEQ ID NO: 56).

128. The pIgR modulator of embodiment 127, wherein the CDR2 comprises the amino acid sequence of AISWSGGSTTYADPVKG (SEQ ID NO: 36).

129. The pIgR modulator of embodiment 127, wherein the CDR2 comprises the amino acid sequence of SWSGGS (SEQ ID NO: 46).

130. The pIgR modulator of embodiment 127, wherein the CDR2 comprises the amino acid sequence of ISWSGGST (SEQ ID NO: 56).

131. The pIgR modulator of any one of embodiments 71 to 82, or embodiments 123 to 130, wherein the VHH domain comprises a complementarity determining region 3 (CDR3) comprising the amino acid sequence of DQRGY (SEQ ID NO: 67) or NDQRGY (SEQ ID NO: 89).

132. The pIgR modulator of embodiment 131, wherein the CDR3 comprises the amino acid sequence of DQRGY (SEQ ID NO: 67).

133. The pIgR modulator of embodiment 131, wherein the CDR3 comprises the amino acid sequence of NDQRGY (SEQ ID NO: 89).

134. The pIgR modulator of any one of embodiments 71 to 82, wherein the VHH domain is comprised of a germline sequence of

(SEQ ID NO: 292)
QVQLVESGGGLVQAGGSLRLSCAVSGRTFSTYRMGWFRQAPGKERSFVAA
ISWSGGSTTYADPVKGRFTISRDNAKNTVYLRMNSLKPEDTAVYYCN.

135. The pIgR modulator of any one of embodiments 71 to 82, or embodiments 123 to 134, wherein the VHH domain is comprised of a J segment, wherein the J segment comprises the sequence of WGQGTLVTVSS (SEQ ID NO: 301).

136. The pIgR modulator of any one of embodiments 71 to 82, wherein the VHH domain is comprised of a sequence of

(SEQ ID NO: 100)
QVQLVESGGGLVQAGGSLRLSCAVSGRTFSTYRMGWFRQAPGKERSFVA
AISWSGGSTTYADPVKGRFTISRDNAKNTVYLRMNSLKPEDTAVYYCND
QRGYWGQGTLVTVSS.

137. The pIgR modulator of any one of embodiments 71 to 82, wherein the VHH domain comprises a complementarity determining region 1 (CDR1) comprising the amino acid sequence of TYRMG (SEQ ID NO: 7), GRTFTTY (SEQ ID NO: 18), or GRTFTTYR (SEQ ID NO: 28).

138. The pIgR modulator of embodiment 137, wherein the CDR1 comprises the amino acid sequence of TYRMG (SEQ ID NO: 7).

139. The pIgR modulator of embodiment 137, wherein the CDR1 comprises the amino acid sequence of GRTFTTY (SEQ ID NO: 18).

140. The pIgR modulator of embodiment 137, wherein the CDR1 comprises the amino acid sequence of GRTFTTYR (SEQ ID NO: 28).

141. The pIgR modulator of any one of embodiments 71 to 82, or embodiments 137 to 140, wherein the VHH domain comprises a complementarity determining region 2 (CDR2) comprising the amino acid sequence of AIRWSGGRTLYADSVKG (SEQ ID NO: 38), RWSGGR (SEQ ID NO: 48), or IRWSGGRT (SEQ ID NO: 58).

142. The pIgR modulator of embodiment 141, wherein the CDR2 comprises the amino acid sequence of AIRWSGGRTLYADSVKG (SEQ ID NO: 38).

143. The pIgR modulator of embodiment 141, wherein the CDR2 comprises the amino acid sequence of RWSGGR (SEQ ID NO: 48).

144. The pIgR modulator of embodiment 141, wherein the CDR2 comprises the amino acid sequence of IRWSGGRT (SEQ ID NO: 58).

145. The pIgR modulator of any one of embodiments 71 to 82, or embodiments 137 to 144, wherein the VHH domain comprises a complementarity determining region 3 (CDR3) comprising the amino acid sequence of DLAEYSGTYSSPADSPAGYDY (SEQ ID NO: 69) or AADLAEYSGTYSSPADSPAGYDY (SEQ ID NO: 91).

146. The pIgR modulator of embodiment 145, wherein the CDR3 comprises the amino acid sequence of DLAEYSGTYSSPADSPAGYDY (SEQ ID NO: 69).

147. The pIgR modulator of embodiment 145, wherein the CDR3 comprises the amino acid sequence of AADLAEYSGTYSSPADSPAGYDY (SEQ ID NO: 91).

148. The pIgR modulator of any one of embodiments 71 to 82, wherein the VHH domain is comprised of a germline sequence of

(SEQ ID NO: 294)
EVQVVESGGGLVQAGGSLRLSCAASGRTFTTYRMGWFRQAPGKEREFVA
AIRWSGGRTLYADSVKGRFTISRDNAKNTAYLQMNNLRPEDTAVYYCAA.

149. The pIgR modulator of any one of embodiments 71 to 82, or embodiments 137 to 148, wherein the VHH domain is comprised of a J segment, wherein the J segment comprises the sequence of YDYWGQGTQVTVSS (SEQ ID NO: 302).

150. The pIgR modulator of any one of embodiments 71 to 82, wherein the VHH domain is comprised of a sequence of

(SEQ ID NO: 102)
EVQVVESGGGLVQAGGSLRLSCAASGRTFTTYRMGWFRQAPGKEREFVAA
IRWSGGRTLYADSVKGRFTISRDNAKNTAYLQMNNLRPEDTAVYYCAADL
AEYSGTYSSPADSPAGYDYWGQGTQVTVSS.

151. The pIgR modulator of any one of embodiments 71 to 82, wherein the VHH domain comprises a complementarity determining region 1 (CDR1) comprising the amino acid sequence of FNTYAMG (SEQ ID NO: 9), GRTLSFNTY (SEQ ID NO: 19), or GRTLSFNTYA (SEQ ID NO: 29).

152. The pIgR modulator of embodiment 151, wherein the CDR1 comprises the amino acid sequence of FNTYAMG (SEQ ID NO: 9).

153. The pIgR modulator of embodiment 151, wherein the CDR1 comprises the amino acid sequence of GRTLSFNTY (SEQ ID NO: 19).

154. The pIgR modulator of embodiment 151, wherein the CDR1 comprises the amino acid sequence of GRTLSFNTYA (SEQ ID NO: 29).

155. The pIgR modulator of any one of embodiments 71 to 82, or embodiments 151 to 154, wherein the VHH domain comprises a complementarity determining region 2 (CDR2) comprising the amino acid sequence of SITWNGGSTSYADSVKG (SEQ ID NO: 39), TWNGGS (SEQ ID NO: 49), or ITWNGGST (SEQ ID NO: 59).

156. The pIgR modulator of embodiment 155, wherein the CDR2 comprises the amino acid sequence of SITWNGGSTSYADSVKG (SEQ ID NO: 39).

157. The pIgR modulator of embodiment 155, wherein the CDR2 comprises the amino acid sequence of TWNGGS (SEQ ID NO: 49).

158. The pIgR modulator of embodiment 155, wherein the CDR2 comprises the amino acid sequence of ITWNGGST (SEQ ID NO: 59).

159. The pIgR modulator of any one of embodiments 71 to 82, or embodiments 151 to 158, wherein the VHH domain comprises a complementarity determining region 3 (CDR3) comprising the amino acid sequence of ARYYVSGTYFPANY (SEQ ID NO: 70) or AAARYYVSGTYFPANY (SEQ ID NO: 92).

160. The pIgR modulator of embodiment 159, wherein the CDR3 comprises the amino acid sequence of ARYYVSGTYFPANY (SEQ ID NO: 70).

161. The pIgR modulator of embodiment 159, wherein the CDR3 comprises the amino acid sequence of AAARYYVSGTYFPANY (SEQ ID NO: 92).

162. The pIgR modulator of any one of embodiments 71 to 82, wherein the VHH domain is comprised of a germline sequence of

(SEQ ID NO: 295)
QVQLVETGGGLVQAGDSLRLSCAASGRTLSFNTYAMGWFRQAPGKEREFV
ASITWNGGSTSYADSVKGRFTITRDNAKNTATLRMNSLQPDDTAVYYCAA.

163. The pIgR modulator of any one of embodiments 71 to 82, or embodiments 151 to 162, wherein the VHH domain is comprised of a J segment, wherein the J segment comprises the sequence of NYWGQGTQVTVSS (SEQ ID NO: 303).

164. The pIgR modulator of any one of embodiments 71 to 82, wherein the VHH domain is comprised of a sequence of

(SEQ ID NO: 103)
QVQLVETGGGLVQAGDSLRLSCAASGRTLSFNTYAMGWFRQAPGKEREFV
ASITWNGGSTSYADSVKGRFTITRDNAKNTATLRMNSLQPDDTAVYYCAA
ARYYVSGTYFPANYWGQGTQVTVSS.

165. The pIgR modulator of any one of embodiments 71 to 82, wherein the VHH domain comprises a complementarity determining region 1 (CDR1) comprising the amino acid sequence of SYRMG (SEQ ID NO: 1), GLTFSSY (SEQ ID NO: 10), or GLTFSSYR (SEQ ID NO: 20).

166. The pIgR modulator of embodiment 165, wherein the CDR1 comprises the amino acid sequence of SYRMG (SEQ ID NO: 1).

167. The pIgR modulator of embodiment 165, wherein the CDR1 comprises the amino acid sequence of GLTFSSY (SEQ ID NO: 10).

168. The pIgR modulator of embodiment 165, wherein the CDR1 comprises the amino acid sequence of GLTFSSYR (SEQ ID NO: 20).

169. The pIgR modulator of any one of embodiments 71 to 82, or embodiments 165 to 168, wherein the VHH domain comprises a complementarity determining region 2 (CDR2) comprising the amino acid sequence of AIDWNGRGTYYRYYADSVKG (SEQ ID NO: 30), DWNGRGTYY (SEQ ID NO: 40), or IDWNGRGTYY (SEQ ID NO: 50).

170. The pIgR modulator of embodiment 169, wherein the CDR2 comprises the amino acid sequence of AIDWNGRGTYYRYYADSVKG (SEQ ID NO: 30).

171. The pIgR modulator of embodiment 169, wherein the CDR2 comprises the amino acid sequence of DWNGRGTYY (SEQ ID NO: 40).

172. The pIgR modulator of embodiment 169, wherein the CDR2 comprises the amino acid sequence of IDWNGRGTYY (SEQ ID NO: 50).

173. The pIgR modulator of any one of embodiments 71 to 82, or embodiments 165 to 172, wherein the VHH domain comprises a complementarity determining region 3 (CDR3) comprising the amino acid sequence of GSIDLNWYGGMDY (SEQ ID NO: 60) or CAAGSIDLNWYGGMDY (SEQ ID NO: 82).

174. The pIgR modulator of embodiment 173, wherein the CDR3 comprises the amino acid sequence of GSIDLNWYGGMDY (SEQ ID NO: 60).

175. The pIgR modulator of embodiment 173, wherein the CDR3 comprises the amino acid sequence of CAAGSIDLNWYGGMDY (SEQ ID NO: 82).

176. The pIgR modulator of any one of embodiments 71 to 82, wherein the VHH domain is comprised of a germline sequence of

(SEQ ID NO: 285)
QVQLVESGGGLVQAGGSLKLACAAPGLTFSSYRMGWFRQAPGQEREFVAA
IDWNGRGTYYRYYADSVKGRSTISRDNAKNTMYLQMNSLKPEDTAVYYC
A.

177. The pIgR modulator of any one of embodiments 71 to 82, or embodiments 165 to 176, wherein the VHH domain is comprised of a J segment, wherein the J segment comprises the sequence of DYWGQGTQVTVSS (SEQ ID NO: 296).

178. The pIgR modulator of any one of embodiments 71 to 82, wherein the VHH domain is comprised of a sequence of

(SEQ ID NO: 93)
QVQLVESGGGLVQAGGSLKLACAAPGLTFSSYRMGWFRQAPGQEREFVAA
IDWNGRGTYYRYYADSVKGRSTISRDNAKNTMYLQMNSLKPEDTAVYYCA
AGSIDLNWYGGMDYWGQGTQVTVSS.

179. The pIgR modulator of any one of embodiments 71 to 82, wherein the VHH domain comprises a complementarity determining region 1 (CDR1) comprising the amino acid sequence of INVMG (SEQ ID NO: 2), GSIFSIN (SEQ ID NO: 11), or GSIFSINV (SEQ ID NO: 21).

180. The pIgR modulator of embodiment 179, wherein the CDR1 comprises the amino acid sequence of INVMG (SEQ ID NO: 2).

181. The pIgR modulator of embodiment 179, wherein the CDR1 comprises the amino acid sequence of GSIFSIN (SEQ ID NO: 11).

182. The pIgR modulator of embodiment 179, wherein the CDR1 comprises the amino acid sequence of GSIFSINV (SEQ ID NO: 21).

183. The pIgR modulator of any one of embodiments 71 to 82, or embodiments 179 to 182, wherein the VHH domain comprises a complementarity determining region 2 (CDR2) comprising the amino acid sequence of RINGGGITHYAESVKG (SEQ ID NO: 31), NGGGI (SEQ ID NO: 41), or INGGGIT (SEQ ID NO: 51).

184. The pIgR modulator of embodiment 183, wherein the CDR2 comprises the amino acid sequence of RINGGGITHYAESVKG (SEQ ID NO: 31).

185. The pIgR modulator of embodiment 183, wherein the CDR2 comprises the amino acid sequence of NGGGI (SEQ ID NO: 41).

186. The pIgR modulator of embodiment 183, wherein the CDR2 comprises the amino acid sequence of INGGGIT (SEQ ID NO: 51).

187. The pIgR modulator of any one of embodiments 71 to 82, or embodiments 179 to 186, wherein the VHH domain comprises a complementarity determining region 3 (CDR3) comprising the amino acid sequence of DVFGSSGYVETY (SEQ ID NO: 62) or KADVFGSSGYVETY (SEQ ID NO: 84).

188. The pIgR modulator of embodiment 187, wherein the CDR3 comprises the amino acid sequence of DVFGSSGYVETY (SEQ ID NO: 62).

189. The pIgR modulator of embodiment 187, wherein the CDR3 comprises the amino acid sequence of KADVFGSSGYVETY (SEQ ID NO: 84).

190. The pIgR modulator of any one of embodiments 71 to 82, wherein the VHH domain is comprised of a germline sequence of

(SEQ ID NO: 287)
QLQLVESGGGLVQPGGSLRLSCAASGSIFSINVMGWYRQAPGKQRELVAR
INGGGITHYAESVKGRFTISRDNAKNTVYLQMNSLKPEDTAAYYCKA.

191. The pIgR modulator of any one of embodiments 71 to 82, or embodiments 179 to 190, wherein the VHH domain is comprised of a J segment, wherein the J segment comprises the sequence of YWGQGTQVTVSS (SEQ ID NO: 298).

192. The pIgR modulator of any one of embodiments 71 to 82, wherein the VHH domain is comprised of a sequence of

(SEQ ID NO: 95)
QLQLVESGGGLVQPGGSLRLSCAASGSIFSINVMGWYRQAPGKQRELVAR
INGGGITHYAESVKGRFTISRDNAKNTVYLQMNSLKPEDTAAYYCKADVF
GSSGYVETYWGQGTQVTVSS.

193. The pIgR modulator of any one of embodiments 71 to 82, wherein the VHH domain comprises a complementarity determining region 1 (CDR1) comprising the amino acid sequence of SNAMG (SEQ ID NO: 3), GTSVSSN (SEQ ID NO: 12), or GTSVSSNA (SEQ ID NO: 22).

194. The pIgR modulator of embodiment 193, wherein the CDR1 comprises the amino acid sequence of SNAMG (SEQ ID NO: 3).

195. The pIgR modulator of embodiment 193, wherein the CDR1 comprises the amino acid sequence of GTSVSSN (SEQ ID NO: 12).

196. The pIgR modulator of embodiment 193, wherein the CDR1 comprises the amino acid sequence of GTSVSSNA (SEQ ID NO: 22).

197. The pIgR modulator of any one of embodiments 71 to 82, or embodiments 193 to 196, wherein the VHH domain comprises a complementarity determining region 2 (CDR2) comprising the amino acid sequence of FIDRIATTTIATSVKG (SEQ ID NO: 32), DRIAT (SEQ ID NO: 42), or IDRIATT (SEQ ID NO: 52).

198. The pIgR modulator of embodiment 197, wherein the CDR2 comprises the amino acid sequence of FIDRIATTTIATSVKG (SEQ ID NO: 32).

199. The pIgR modulator of embodiment 197, wherein the CDR2 comprises the amino acid sequence of DRIAT (SEQ ID NO: 42).

200. The pIgR modulator of embodiment 197, wherein the CDR2 comprises the amino acid sequence of IDRIATT (SEQ ID NO: 52).

201. The pIgR modulator of any one of embodiments 71 to 82, or embodiments 193 to 200, wherein the VHH domain comprises a complementarity determining region 3 (CDR3) comprising the amino acid sequence of PLTAR (SEQ ID NO: 63) or NHPLTAR (SEQ ID NO: 85).

202. The pIgR modulator of embodiment 201, wherein the CDR3 comprises the amino acid sequence of PLTAR (SEQ ID NO: 63).

203. The pIgR modulator of embodiment 201, wherein the CDR3 comprises the amino acid sequence of NHPLTAR (SEQ ID NO: 85).

204. The pIgR modulator of any one of embodiments 71 to 82, wherein the VHH domain is comprised of a germline sequence of

(SEQ ID NO: 288)
EVQVVESGGGLVQAGGSLRLSCAVSGTSVSSNAMGWYRQAPGKQREWVGF
IDRIATTTIATSVKGRFAITRDNAKNTVYLQMSGLKPEDTAVYYCN.

205. The pIgR modulator of any one of embodiments 71 to 82, or embodiments 193 to 204, wherein the VHH domain is comprised of a J segment, wherein the J segment comprises the sequence of WGQGTQVTVSS (SEQ ID NO: 299).

206. The pIgR modulator of any one of embodiments 71 to 82, wherein the VHH domain is comprised of a sequence of

(SEQ ID NO: 96)
EVQVVESGGGLVQAGGSLRLSCAVSGTSVSSNAMGWYRQAPGKQREWVGF
IDRIATTTIATSVKGRFAITRDNAKNTVYLQMSGLKPEDTAVYYCNHPLT
ARWGQGTQVTVSS.

207. The pIgR modulator of any one of embodiments 71 to 82, wherein the VHH domain comprises a complementarity determining region 1 (CDR1) comprising the amino acid sequence of SYAMG (SEQ ID NO: 4), GRTFSSY (SEQ ID NO: 13), or GRTFSSYA (SEQ ID NO: 23).

208. The pIgR modulator of embodiment 207, wherein the CDR1 comprises the amino acid sequence of SYAMG (SEQ ID NO: 4).

209. The pIgR modulator of embodiment 207, wherein the CDR1 comprises the amino acid sequence of GRTFSSY (SEQ ID NO: 13).

210. The pIgR modulator of embodiment 207, wherein the CDR1 comprises the amino acid sequence of GRTFSSYA (SEQ ID NO: 23).

211. The pIgR modulator of any one of embodiments 71 to 82, or embodiments 207 to 210, wherein the VHH domain comprises a complementarity determining region 2 (CDR2) comprising the amino acid sequence of AITWNGGTTYYADSVKG (SEQ ID NO: 33), TWNGGT (SEQ ID NO: 43), or ITWNGGTT (SEQ ID NO: 53).

212. The pIgR modulator of embodiment 211, wherein the CDR2 comprises the amino acid sequence of AITWNGGTTYYADSVKG (SEQ ID NO: 33).

13. The pIgR modulator of embodiment 211, wherein the CDR2 comprises the amino acid sequence of TWNGGT (SEQ ID NO: 43).

214. The pIgR modulator of embodiment 211, wherein the CDR2 comprises the amino acid sequence of ITWNGGTT (SEQ ID NO: 53).

215. The pIgR modulator of any one of embodiments 71 to 82, or embodiments 207 to 214, wherein the VHH domain comprises a complementarity determining region 3 (CDR3) comprising the amino acid sequence of DPFNQGY (SEQ ID NO: 64) or AADPFNQGY (SEQ ID NO: 86).

216. The pIgR modulator of embodiment 215, wherein the CDR3 comprises the amino acid sequence of DPFNQGY (SEQ ID NO: 64).

217. The pIgR modulator of embodiment 215, wherein the CDR3 comprises the amino acid sequence of AADPFNQGY (SEQ ID NO: 86).

218. The pIgR modulator of any one of embodiments 71 to 82, wherein the VHH domain is comprised of a germline sequence of

(SEQ ID NO: 289)
QVQLVESGGGLVQAGGSLRLSCAASGRTFSSYAMGWFRQAPGKEREFVAA
ITWNGGTTYYADSVKGRFTISRDNAKNTVYLQMNSLKPEDTAVYYCAA.

219. The pIgR modulator of any one of embodiments 71 to 82, or embodiments 207 to 218, wherein the VHH domain is comprised of a J segment, wherein the J segment comprises the sequence of YWGQGTQVTVSS (SEQ ID NO: 298).

220. The pIgR modulator of any one of embodiments 71 to 82, wherein the VHH domain is comprised of a sequence of

(SEQ ID NO: 97)
QVQLVESGGGLVQAGGSLRLSCAASGRTFSSYAMGWFRQAPGKEREFVAA
ITWNGGTTYYADSVKGRFTISRDNAKNTVYLQMNSLKPEDTAVYYCAADP
FNQGYWGQGTQVTVSS.

221. The pIgR modulator of any one of embodiments 71 to 82, wherein the VHH domain comprises a complementarity determining region 1 (CDR1) comprising the amino acid sequence of INVMG (SEQ ID NO: 2), RSIGSIN (SEQ ID NO: 15), or RSIGSINV (SEQ ID NO: 25).

222. The pIgR modulator of embodiment 221, wherein the CDR1 comprises the amino acid sequence of INVMG (SEQ ID NO: 2).

223. The pIgR modulator of embodiment 221, wherein the CDR1 comprises the amino acid sequence of RSIGSIN (SEQ ID NO: 15).

224. The pIgR modulator of embodiment 221, wherein the CDR1 comprises the amino acid sequence of RSIGSINV (SEQ ID NO: 25).

225. The pIgR modulator of any one of embodiments 71 to 82, or embodiments 221 to 224, wherein the VHH domain comprises a complementarity determining region 2 (CDR2) comprising the amino acid sequence of RITGGGSTHYAESVKG (SEQ ID NO: 35), TGGGS (SEQ ID NO: 45), or ITGGGST (SEQ ID NO: 55).

226. The pIgR modulator of embodiment 225, wherein the CDR2 comprises the amino acid sequence of RITGGGSTHYAESVKG (SEQ ID NO: 35).

227. The pIgR modulator of embodiment 225, wherein the CDR2 comprises the amino acid sequence of TGGGS (SEQ ID NO: 45).

228. The pIgR modulator of embodiment 225, wherein the CDR2 comprises the amino acid sequence of ITGGGST (SEQ ID NO: 55).

229. The pIgR modulator of any one of embodiments 71 to 82, or embodiments 221 to 228, wherein the VHH domain comprises a complementarity determining region 3 (CDR3) comprising the amino acid sequence of MVNPIITAWGTIGVREIPDYDY (SEQ ID NO: 66) or ASMVNPIITAWGTIGVREIPDYDY (SEQ ID NO: 88).

230. The pIgR modulator of embodiment 229, wherein the CDR3 comprises the amino acid sequence of MVNPIITAWGTIGVREIPDYDY (SEQ ID NO: 66).

231. The pIgR modulator of embodiment 229, wherein the CDR3 comprises the amino acid sequence of ASMVNPIITAWGTIGVREIPDYDY (SEQ ID NO: 88).

232. The pIgR modulator of any one of embodiments 71 to 82, wherein the VHH domain is comprised of a germline sequence of

(SEQ ID NO: 291)
EVQVVESGGGLVQAGGSLRLACVASRSIGSINVMGWYRQAPGKQRDLVAR
ITGGGSTHYAESVKGRFTISRDNAKNTVYLQMNSLEPEDTAVYYC.

233. The pIgR modulator of any one of embodiments 71 to 82, or embodiments 221 to 232, wherein the VHH domain is comprised of a J segment, wherein the J segment comprises the sequence of DYDYWGQGTQVTVSS (SEQ ID NO: 300).

234. The pIgR modulator of any one of embodiments 71 to 82, wherein the VHH domain is comprised of a sequence of

(SEQ ID NO: 99)
EVQVVESGGGLVQAGGSLRLACVASRSIGSINVMGWYRQAPGKQRDLVAR
ITGGGSTHYAESVKGRFTISRDNAKNTVYLQMNSLEPEDTAVYYCASMVN
PIITAWGTIGVREIPDYDYWGQGTQVTVSS.

235. The pIgR modulator of any one of embodiments 71 to 82, wherein the VHH domain comprises a complementarity determining region 1 (CDR1) comprising the amino acid sequence of RYAMG (SEQ ID NO: 8), GFTFTRY (SEQ ID NO: 17), or GFTFTRYA (SEQ ID NO: 27).

236. The pIgR modulator of embodiment 235, wherein the CDR1 comprises the amino acid sequence of RYAMG (SEQ ID NO: 8).

237. The pIgR modulator of embodiment 235, wherein the CDR1 comprises the amino acid sequence of GFTFTRY (SEQ ID NO: 17).

238. The pIgR modulator of embodiment 235, wherein the CDR1 comprises the amino acid sequence of GFTFTRYA (SEQ ID NO: 27).

239. The pIgR modulator of any one of embodiments 71 to 82, or embodiments 235 to 238, wherein the VHH domain comprises a complementarity determining region 2 (CDR2) comprising the amino acid sequence of AISWSGSSAGYGDSVKG (SEQ ID NO: 37), SWSGSS (SEQ ID NO: 47), or ISWSGSSA (SEQ ID NO: 57).

240. The pIgR modulator of embodiment 239, wherein the CDR2 comprises the amino acid sequence of AISWSGSSAGYGDSVKG (SEQ ID NO: 37).

241. The pIgR modulator of embodiment 239, wherein the CDR2 comprises the amino acid sequence of SWSGSS (SEQ ID NO: 47).

242. The pIgR modulator of embodiment 239, wherein the CDR2 comprises the amino acid sequence of ISWSGSSA (SEQ ID NO: 57).

243. The pIgR modulator of any one of embodiments 71 to 82, or embodiments 235 to 242, wherein the VHH domain comprises a complementarity determining region 3 (CDR3) comprising the amino acid sequence of DPFNQGY (SEQ ID NO: 64) or AADPFNQGY (SEQ ID NO: 86).

244. The pIgR modulator of embodiment 243, wherein the CDR3 comprises the amino acid sequence of DPFNQGY (SEQ ID NO: 64).

245. The pIgR modulator of embodiment 243, wherein the CDR3 comprises the amino acid sequence of AADPFNQGY (SEQ ID NO: 86).

246. The pIgR modulator of any one of embodiments 71 to 82, wherein the VHH domain is comprised of a germline sequence of

(SEQ ID NO: 293)
EVQVVESGGGLVQAGGSLRLSCAASGFTFTRYAMGWFRQAPGKERSFVAA
ISWSGSSAGYGDSVKGRFTISRDNAKNTLYLQMNSLKPEDTAVYYCA.

247. The pIgR modulator of any one of embodiments 71 to 82, or embodiments 235 to 246, wherein the VHH domain is comprised of a J segment, wherein the J segment comprises the sequence of YWGQGTQVTVSS (SEQ ID NO: 298).

248. The pIgR modulator of any one of embodiments 71 to 82, wherein the VHH domain is comprised of a sequence of

(SEQ ID NO: 101)
EVQVVESGGGLVQAGGSLRLSCAASGFTFTRYAMGWFRQAPGKERSFVAA
ISWSGSSAGYGDSVKGRFTISRDNAKNTLYLQMNSLKPEDTAVYYCAADP
FNQGYWGQGTQVTVSS.

249. A pharmaceutical composition comprising the pIgR modulator of any one of embodiments 71 to 248, and a pharmaceutically acceptable carrier.

250. The pharmaceutical composition according to embodiment 249, wherein the composition is formulated for parenteral administration.

251. The pharmaceutical composition according to embodiment 249, wherein the composition is formulated for intravenous, intramuscular, subcutaneous, or intradermal administration.

252. A VHH domain that binds to pIgR, including wherein optionally the VHH domain binds to an extracellular domain of the pIgR, including a VHH domain that binds to an extracellular domain 1, an extracellular domain 2, an extracellular domain 1-2, an extracellular domain 3, an extracellular domain 2-3, an extracellular domain 4-5, or an extracellular domain 5 of pIgR, wherein optionally pIgR is human pIgR or mouse pIgR, wherein optionally the VHH domain does not detectably bind to the amino acid sequence of

(SEQ ID NO: 143)
EKAVADTRDQADGSRASVDSGSSEEQGGSSR,
(SEQ ID NO: 144)
EREIQNVGDQAQENRASGDAGSADGQSRSSSSK
or
(SEQ ID NO: 145)
EREIQNVRDQAQENRASGDAGSADGQSRSSSSK.

253. The VHH domain of embodiment 252, wherein the VHH domain competes with IgA binding to the pIgR.

254. The VHH domain of embodiment 252, wherein the VHH domain promotes IgA binding to the pIgR.

255. The VHH domain of any one of embodiments 252-254, wherein the K_{D} of the binding of the VHH domain to pIgR is from about 4 to about 525 nM.

256. The VHH domain of any one of embodiments 252-255, wherein the K_{D} of the binding of the VHH domain to pIgR is less than about 50 nM.

257. The VHH domain of any one of embodiments 252-256, wherein the K_{D} of the binding of the VHH domain to pIgR is from about 4 to about 34 nM.

258. The VHH domain of any one of embodiments 252-257, wherein the Tₘ of the VHH domain is from about 53 to about 77° C.

259. The VHH domain of any one of embodiments 252-258, wherein the Tₘ of the VHH domain is from 53.9 to 76.4° C.

260. The VHH domain of any one of embodiments 252-259, wherein the VHH domain comprises a CDR1 sequence of SYRMG (SEQ ID NO: 1), INVMG (SEQ ID NO: 2), SNAMG (SEQ ID NO: 3), SYAMG (SEQ ID NO: 4), SDAMG (SEQ ID NO: 5), INVMG (SEQ ID NO: 6), TYRMG (SEQ ID NO: 7), RYAMG (SEQ ID NO: 8), FTTYRMG (SEQ ID NO: 258), TYRMG (SEQ ID NO: 259), FNTYAMG (SEQ ID NO: 9), GLTFSSY (SEQ ID NO: 10), GSIFSIN (SEQ ID NO: 11), GTSVSSN (SEQ ID NO: 12), GRTFSSY (SEQ ID NO: 13), GSSVSSD (SEQ ID NO: 14), RSIGSIN (SEQ ID NO: 15), GRTFSTY (SEQ ID NO: 16), GFTFTRY (SEQ ID NO: 17), GRTFTTY (SEQ ID NO: 18), GRTLSFNTY (SEQ ID NO: 19), GLTFSSYR (SEQ ID NO: 20), GSIFSINV (SEQ ID NO: 21), GTSVSSNA (SEQ ID NO: 22), GRTFSSYA (SEQ ID NO: 23), GSSVSSDA (SEQ ID NO: 24), RSIGSINV (SEQ ID NO: 25), GRTFSTYR (SEQ ID NO: 26), GFTFTRYA (SEQ ID NO: 27), GRTFTTYR (SEQ ID NO: 28), GRTLSFNTYA (SEQ ID NO: 29), GLTFSSYRMG (SEQ ID NO: 154), GSIFSINVMG (SEQ ID NO: 155), GTSVSSNAMG (SEQ ID NO: 156), GRTFSSYAMG (SEQ ID NO: 157), GSSVSSDAMG (SEQ ID NO: 158), RSIGSINVMG (SEQ ID NO: 159), GRTFSTYRMG (SEQ ID NO: 160), GFTFTRYAMG (SEQ ID NO: 161), GRTFTTYRMG (SEQ ID NO: 162), GRTLSFNTYAMG (SEQ ID NO: 163), SSYRMG (SEQ ID NO: 164), SINVMG (SEQ ID NO: 165), SSNAMG (SEQ ID NO: 166), SSYAMG (SEQ ID NO: 167), SSDAMG (SEQ ID NO: 168), SINVMG (SEQ ID NO: 169), STYRMG (SEQ ID NO: 170), TRYAMG (SEQ ID NO: 171), TTYRMG (SEQ ID NO: 172), SFNTYAMG (SEQ ID NO: 173), GLTFSSYRMG (SEQ ID NO: 174), GSIFSINVMG (SEQ ID NO: 175), GTSVSSNAMG (SEQ ID NO: 176), GRTFSSYAMG (SEQ ID NO: 177), GSSVSSDAMG (SEQ ID NO: 178), RSIGSINVMG (SEQ ID NO: 179), GRTFSTYRMG (SEQ ID NO: 180), GFTFTRYAMG (SEQ ID NO: 181), GRTFTTYRMG (SEQ ID NO: 182), or GRTLSFNTYAMG (SEQ ID NO: 183).

261. The VHH domain of any one of embodiments 252-260, wherein the VHH domain comprises a CDR2 sequence of AIDWNGRGTYYRYYADSVKG (SEQ ID NO: 30), RINGGGITHYAESVKG (SEQ ID NO: 31), FIDRIATTTIATSVKG (SEQ ID NO: 32), AITWNGGTTYYADSVKG (SEQ ID NO: 33), FISGGGTTTYADSVKG (SEQ ID NO: 34), RITGGGSTHYAESVKG (SEQ ID NO: 35), AISWSGGSTTYADPVKG (SEQ ID NO: 36), AISWSGSSAGYGDSVKG (SEQ ID NO: 37), AIRWSGGRTLYADSVKG (SEQ ID NO: 38), SITWNGGSTSYADSVKG (SEQ ID NO: 39), DWNGRGTYY (SEQ ID NO: 40), WNGRGTY (SEQ ID NO: 260), NGGGI (SEQ ID NO: 41), GGG (SEQ ID NO: 261), DRIAT (SEQ ID NO: 42), RIA (SEQ ID NO: 262), TWNGGT (SEQ ID NO: 43), WNGG (SEQ ID NO: 263), SGGGT (SEQ ID NO: 44), GGG (SEQ ID NO: 264), TGGGS (SEQ ID NO: 45), GGG (SEQ ID NO: 265), SWSGGS (SEQ ID NO: 46), WSGG (SEQ ID NO: 266), SWSGSS (SEQ ID NO: 47), WSGS (SEQ ID NO: 267), RWSGGR (SEQ ID NO: 48), WSGG (SEQ ID NO: 268), TWNGGS (SEQ ID NO: 49), WNGG (SEQ ID NO: 269), IDWNGRGTYY (SEQ ID NO: 50), IDWNGRGTYYR (SEQ ID NO: 270), INGGGIT (SEQ ID NO: 51), IDRIATT (SEQ ID NO: 52), ITWNGGTT (SEQ ID NO: 53), ISGGGTT (SEQ ID NO: 54), ITGGGST (SEQ ID NO: 55), ISWSGGST (SEQ ID NO: 56), ISWSGSSA (SEQ ID NO: 57), IRWSGGRT (SEQ ID NO: 58), ITWNGGST (SEQ ID NO: 59), AIDWNGRGTYYRYYADSVKG (SEQ ID NO: 184), RINGGGITHYAESVKG (SEQ ID NO: 185), FIDRIATTTIATSVKG (SEQ ID NO: 186), AITWNGGTTYYADSVKG (SEQ ID NO: 187), FISGGGTTTYADSVKG (SEQ ID NO: 188), RITGGGSTHYAESVKG (SEQ ID NO: 189), AISWSGGSTTYADPVKG (SEQ ID NO: 190), AISWSGSSAGYGDSVKG (SEQ ID NO: 191), AIRWSGGRTLYADSVKG (SEQ ID NO: 192), SITWNGGSTSYADSVKG (SEQ ID NO: 193), FVAAIDWNGRGTYYRY (SEQ ID NO: 194), LVARITGGGITH (SEQ ID NO: 195), WVGFIDRIATTT (SEQ ID NO: 196), FVAAITWNGGTTY (SEQ ID NO: 197), WVAFISGGGTTT (SEQ ID NO: 198), LVARITGGGSTH (SEQ ID NO: 199), FVAAISWSGGSTT (SEQ ID NO: 200), FVAAISWSGSSAG (SEQ ID NO: 201), FVAAIRWSGGRTL (SEQ ID NO: 202), FVASITWNGGSTS (SEQ ID NO: 203), AIDWNGRGTYYRY (SEQ ID NO: 204), RINGGGITH (SEQ ID NO: 205), FIDRIATTT (SEQ ID NO: 206), AITWNGGTTY (SEQ ID NO: 207), FISGGGTTT (SEQ ID NO: 208), RITGGGSTH (SEQ ID NO: 209), AISWSGGSTT (SEQ ID NO: 210), AISWSGSSAG (SEQ ID NO: 211), AIRWSGGRTL (SEQ ID NO: 212), or SITWNGGSTS (SEQ ID NO: 213).

262. The VHH domain of any one of embodiments 252-261, wherein the VHH domain comprises a CDR3 sequence of GSIDLNWYGGMDY (SEQ ID NO: 60), TTVLTDPRVLNEYAT (SEQ ID NO: 61), DVFGSSGYVETY (SEQ ID NO: 62), PLTAR (SEQ ID NO: 63), DPFNQGY (SEQ ID NO: 64), PLTSR (SEQ ID NO: 65), MVNPIITAWGTIGVREIPDYDY (SEQ ID NO: 66), DQRGY (SEQ ID NO: 67), QRGY (SEQ ID NO: 271), DPFNQGY (SEQ ID NO: 68), DLAEYSGTYSSPADSPAGYDY (SEQ ID NO: 69), ARYYVSGTYFPANY (SEQ ID NO: 70), GSIDLNWYGGMDY (SEQ ID NO: 71), SIDLNWYGGMD (SEQ ID NO: 272), TTVLTDPRVLNEYAT (SEQ ID NO: 72), TVLTDPRVLNEYA (SEQ ID NO: 273), DVFGSSGYVETY (SEQ ID NO: 73), VFGSSGYVET (SEQ ID NO: 274), PLTAR (SEQ ID NO: 74), LTA (SEQ ID NO: 275), DPFNQGY (SEQ ID NO: 75), PFNQG (SEQ ID NO: 276), PLTSR (SEQ ID NO: 76), LTS (SEQ ID NO: 277), MVNPIITAWGTIGVREIPDYDY (SEQ ID NO: 77), VNPIITAWGTIGVREIPDYD (SEQ ID NO: 278), DQRGY (SEQ ID NO: 78), RG (SEQ ID NO: 279), DPFNQGY (SEQ ID NO: 79), PFNQG (SEQ ID NO: 280), DLAEYSGTYSSPADSPAGYDY (SEQ ID NO: 80), LAEYSGTYSSPADSPAGYD (SEQ ID NO: 281), ARYYVSGTYFPANY (SEQ ID NO: 81), RYYVSGTYFPAN (SEQ ID NO: 282), CAAGSIDLNWYGGMDY (SEQ ID NO: 82), AAGSIDLNWYGGMDY (SEQ ID NO: 283), CAATTVLTDPRVLNEYAT (SEQ ID NO: 83), AATTVLTDPRVLNEYAT (SEQ ID NO: 284), KADVFGSSGYVETY (SEQ ID NO: 84), NHPLTAR (SEQ ID NO: 85), AADPFNQGY (SEQ ID NO: 86), NHPLTSR (SEQ ID NO: 87), ASMVNPIITAWGTIGVREIPDYDY (SEQ ID NO: 88), NDQRGY (SEQ ID NO: 89), AADPFNQGY (SEQ ID NO: 90), AADLAEYSGTYSSPADSPAGYDY (SEQ ID NO: 91), AAARYYVSGTYFPANY (SEQ ID NO: 92), GSIDLNWYGGMDY (SEQ ID NO: 214), TTVLTDPRVLNEYAT (SEQ ID NO: 215), DVFGSSGYVETY (SEQ ID NO: 216), PLTAR (SEQ ID NO: 217), DPFNQGY (SEQ ID NO: 218), PLTSR (SEQ ID NO: 219), MVNPIITAWGTIGVREIPDYDY (SEQ ID NO: 220), QRGY (SEQ ID NO: 221), DPFNQGY (SEQ ID NO: 222), DLAEYSGTYSSPADSPAGYDY (SEQ ID NO: 223), ARYYVSGTYFPANY (SEQ ID NO: 224), AAGSIDLNWYGGMD (SEQ ID NO: 225), AATTVLTDPRVLNEYA (SEQ ID NO: 226), KADVFGSSGYVET (SEQ ID NO: 227), NHPLTA (SEQ ID NO: 228), AADPFNQG (SEQ ID NO: 229), NHPLTS (SEQ ID NO: 230), ASMVNPIITAWGTIGVREIPDYD (SEQ ID NO: 231), NDQRG (SEQ ID NO: 232), AADPFNQG (SEQ ID NO: 233), AADLAEYSGTYSSPADSPAGYD (SEQ ID NO: 234), AAARYYVSGTYFPAN (SEQ ID NO: 235), GSIDLNWYGGMDY (SEQ ID NO: 236), TTVLTDPRVLNEYAT (SEQ ID NO: 237), DVFGSSGYVETY (SEQ ID NO: 238), PLTAR (SEQ ID NO: 239), DPFNQGY (SEQ ID NO: 240), PLTSR (SEQ ID NO: 241), MVNPIITAWGTIGVREIPDYDY (SEQ ID NO: 242), QRGY (SEQ ID NO: 243), DPFNQGY (SEQ ID NO: 244), DLAEYSGTYSSPADSPAGYDY (SEQ ID NO: 245), or ARYYVSGTYFPANY (SEQ ID NO: 246), including wherein optionally the VHH domain comprises a CDR1 sequence, a CDR2 sequence, and a CDR3 sequence of the VHH domain selected from the group consisting of:
a) VHH1:
   i) the CDR1 sequence of SYRMG (SEQ ID NO: 1), the CDR2 sequence of AIDWNGRGTYYRYYADSVKG (SEQ ID NO: 30), and the CDR3 sequence of GSIDLNWYGGMDY (SEQ ID NO: 60);
   ii) the CDR1 sequence of GLTFSSY (SEQ ID NO: 10), the CDR2 sequence of DWNGRGTYY (SEQ ID NO: 40) or WNGRGTY (SEQ ID NO: 260), and the CDR3 sequence of GSIDLNWYGGMDY (SEQ ID NO: 71) or SIDLNWYGGMD (SEQ ID NO: 272);
   iii) the CDR1 sequence of GLTFSSYR (SEQ ID NO: 20), the CDR2 sequence of IDWNGRGTYY (SEQ ID NO: 50) or IDWNGRGTYYR (SEQ ID NO: 270), and the CDR3 sequence of CAAGSIDLNWYGGMDY (SEQ ID NO: 82) or AAGSIDLNWYGGMDY (SEQ ID NO: 283);
   iv) the CDR1 sequence of GLTFSSYRMG (SEQ ID NO: 154), the CDR 2 sequence of AIDWNGRGTYYRYYADSVKG (SEQ ID NO: 184), and the CDR3 sequence of GSIDLNWYGGMDY (SEQ ID NO: 214);
   v) the CDR1 sequence of SSYRMG (SEQ ID NO: 164), the CDR2 sequence of FVAAIDWNGRGTYYRY (SEQ ID NO: 194), and the CDR3 sequence of AAGSIDLNWYGGMD (SEQ ID NO: 225); or
   vi) the CDR1 sequence of GLTFSSYRMG (SEQ ID NO: 174), the CDR2 sequence of AIDWNGRGTYYRY (SEQ ID NO: 204), and the CDR3 sequence of GSIDLNWYGGMDY (SEQ ID NO: 236);
b) VHH2:
   i) the CDR1 sequence of SYRMG (SEQ ID NO: 1), the CDR2 sequence of AIDWNGRGTYYRYYADSVKG (SEQ ID NO: 30), and the CDR3 sequence of TTVLTDPRVLNEYAT (SEQ ID NO: 61);
   ii) the CDR1 sequence of GLTFSSY (SEQ ID NO: 10), the CDR2 sequence of DWNGRGTYY (SEQ ID NO: 40) or WNGRGTY (SEQ ID NO: 260), and the CDR3 sequence of TTVLTDPRVLNEYAT (SEQ ID NO: 72) or TVLTDPRVLNEYA (SEQ ID NO: 273);
   iii) the CDR1 sequence of GLTFSSYR (SEQ ID NO: 20), the CDR2 sequence of IDWNGRGTYY (SEQ ID NO: 50) or IDWNGRGTYYR (SEQ ID NO: 270), and the CDR3 sequence of CAATTVLTDPRVLNEYAT (SEQ ID NO: 83) or AATTVLTDPRVLNEYAT (SEQ ID NO: 284);
   iv) the CDR1 sequence of GLTFSSYRMG (SEQ ID NO: 154), the CDR2 sequence of AIDWNGRGTYYRYYADSVKG (SEQ ID NO: 184), and the CDR3 sequence of TTVLTDPRVLNEYAT (SEQ ID NO: 215);
   v) the CDR1 sequence of SSYRMG (SEQ ID NO: 164), the CDR2 sequence of FVAAIDWNGRGTYYRY (SEQ ID NO: 194), and the CDR3 sequence of AATTVLTDPRVLNEYA (SEQ ID NO: 226); or
   vi) the CDR1 sequence of GLTFSSYRMG (SEQ ID NO: 174), the CDR2 sequence of AIDWNGRGTYYRY (SEQ ID NO: 204), and the CDR3 sequence of TTVLTDPRVLNEYAT (SEQ ID NO: 237);
c) VHH3:
   i) the CDR1 sequence of INVMG (SEQ ID NO: 2), the CDR2 sequence of RINGGGITHYAESVKG (SEQ ID NO: 31), and the CDR3 sequence of DVFGSSGYVETY (SEQ ID NO: 62);
   ii) the CDR1 sequence of GSIFSIN (SEQ ID NO: 11), the CDR2 sequence of NGGGI (SEQ ID NO: 41) or GGG (SEQ ID NO: 261), and the CDR3 sequence of DVFGSSGYVETY (SEQ ID NO: 73) or VFGSSGYVET (SEQ ID NO: 274);
   iii) the CDR1 sequence of GSIFSINV (SEQ ID NO: 21), the CDR2 sequence of INGGGIT (SEQ ID NO: 51), and the CDR3 sequence of KADVFGSSGYVETY (SEQ ID NO: 84);
   iv) the CDR1 sequence of GSIFSINVMG (SEQ ID NO: 155), the CDR2 sequence of RINGGGITHYAESVKG (SEQ ID NO: 185), and the CDR3 sequence of DVFGSSGYVETY (SEQ ID NO: 216);
   v) the CDR1 sequence of SINVMG (SEQ ID NO: 165), the CDR2 sequence of LVARINGGGITH (SEQ ID NO: 195), and the CDR3 sequence of KADVFGSSGYVET (SEQ ID NO: 227); or
   vi) the CDR1 sequence of GSIFSINVMG (SEQ ID NO: 175), the CDR2 sequence of RINGGGITH (SEQ ID NO: 205), and the CDR3 sequence of DVFGSSGYVETY (SEQ ID NO: 238);
d) VHH4:
   i) the CDR1 sequence of SNAMG (SEQ ID NO: 3), the CDR2 sequence of FIDRIATTTIATSVKG (SEQ ID NO: 32), and the CDR3 sequence of PLTAR (SEQ ID NO: 63);
   ii) the CDR1 sequence of GTSVSSN (SEQ ID NO: 12), the CDR2 sequence of DRIAT (SEQ ID NO: 42) or RIA (SEQ ID NO: 262), and the CDR3 sequence of PLTAR (SEQ ID NO: 74) or LTA (SEQ ID NO: 275);
   iii) the CDR1 sequence of GTSVSSNA (SEQ ID NO: 22), the CDR2 sequence of IDRIATT (SEQ ID NO: 52), and the CDR3 sequence of NHPLTAR (SEQ ID NO: 85);
   iv) the CDR1 sequence of GTSVSSNAMG (SEQ ID NO: 156), the CDR2 sequence of FIDRIATTTIATSVKG (SEQ ID NO: 186), and the CDR3 sequence of PLTAR (SEQ ID NO: 217);
   v) the CDR1 sequence of SSNAMG (SEQ ID NO: 166), the CDR2 sequence of WVGFIDRIATTT (SEQ ID NO: 196), and the CDR3 sequence of NHPLTA (SEQ ID NO: 228); or
   vi) the CDR1 sequence of GTSVSSNAMG (SEQ ID NO: 176), the CDR2 sequence of FIDRIATTT (SEQ ID NO: 206), and the CDR3 sequence of PLTAR (SEQ ID NO: 239);
e) VHH5:
   i) the CDR1 sequence of SYAMG (SEQ ID NO: 4), the CDR2 sequence of AITWNGGTTYYADSVKG (SEQ ID NO: 33), and the CDR3 sequence of DPFNQGY (SEQ ID NO: 64);
   ii) the CDR1 sequence of GRTFSSY (SEQ ID NO: 13), the CDR2 sequence of TWNGGT (SEQ ID NO: 43) or WNGG (SEQ ID NO: 263), and the CDR3 sequence of DPFNQGY (SEQ ID NO: 75) or PFNQG (SEQ ID NO: 276);
   iii) the CDR1 sequence of GRTFSSYA (SEQ ID NO: 23), the CDR2 sequence of ITWNGGTT (SEQ ID NO: 53), and the CDR3 sequence of AADPFNQGY (SEQ ID NO: 86);
   iv) the CDR1 sequence of GRTFSSYAMG (SEQ ID NO: 157), the CDR2 sequence of AITWNGGTTYYADSVKG (SEQ ID NO: 187), and the CDR3 sequence of DPFNQGY (SEQ ID NO: 218);
   v) the CDR1 sequence of SSYAMG (SEQ ID NO: 167), the CDR2 sequence of FVAAITWNGGTTY (SEQ ID NO: 197), and the CDR3 sequence of AADPFNQG (SEQ ID NO: 229); or
   vi) the CDR1 sequence of GRTFSSYAMG (SEQ ID NO: 177), the CDR2 sequence of AITWNGGTTY (SEQ ID NO: 207), and the CDR3 sequence of DPFNQGY (SEQ ID NO: 240);
f) VHH6:
   i) the CDR1 sequence of SDAMG (SEQ ID NO: 5), the CDR2 sequence of FISGGGTTTYADSVKG (SEQ ID NO: 34), and the CDR3 sequence of PLTSR (SEQ ID NO: 65);
   ii) the CDR1 sequence of GSSVSSD (SEQ ID NO: 14), the CDR2 sequence of SGGGT (SEQ ID NO: 44) or GGG (SEQ ID NO: 264), and the CDR3 sequence of PLTSR (SEQ ID NO: 76) or LTS (SEQ ID NO: 277);
   iii) the CDR1 sequence of GSSVSSDA (SEQ ID NO: 24), the CDR2 sequence of ISGGGTT (SEQ ID NO: 54), and the CDR3 sequence of NHPLTSR (SEQ ID NO: 87);
   iv) the CDR1 sequence of GSSVSSDAMG (SEQ ID NO: 158), the CDR2 sequence of FISGGGTTTYADSVKG (SEQ ID NO: 188), and the CDR3 sequence of PLTSR (SEQ ID NO: 219);
   v) the CDR1 sequence of SSDAMG (SEQ ID NO: 168), the CDR2 sequence of WVAFISGGGTTT (SEQ ID NO: 198), and the CDR3 sequence of NHPLTS (SEQ ID NO: 230); or
   vi) the CDR1 sequence of GSSVSSDAMG (SEQ ID NO: 178), the CDR2 sequence of FISGGGTTT (SEQ ID NO: 208), and the CDR3 sequence of PLTSR (SEQ ID NO: 241);
g) VHH7:
   i) the CDR1 sequence of INVMG (SEQ ID NO: 6), the CDR2 sequence of RITGGGSTHYAESVKG (SEQ ID NO: 35), and the CDR3 sequence of MVNPIITAWGTIGVREIPDYDY (SEQ ID NO: 66);
   ii) the CDR1 sequence of RSIGSIN (SEQ ID NO: 15), the CDR2 sequence of TGGGS (SEQ ID NO: 45) or GGG (SEQ ID NO: 265), and the CDR3 sequence of MVNPIITAWGTIGVREIPDYDY (SEQ ID NO: 77) or VNPIITAWGTIGVREIPDYD (SEQ ID NO: 278);
   iii) the CDR1 sequence of RSIGSINV (SEQ ID NO: 25), the CDR2 sequence of ITGGGST (SEQ ID NO: 55), and the CDR3 sequence of ASMVNPIITAWGTIGVREIPDYDY (SEQ ID NO: 88);
   iv) the CDR1 sequence of RSIGSINVMG (SEQ ID NO: 159), the CDR2 sequence of RITGGGSTHYAESVKG (SEQ ID NO: 189), and the CDR3 sequence of MVNPIITAWGTIGVREIPDYDY (SEQ ID NO: 220);
   v) the CDR1 sequence of SINVMG (SEQ ID NO: 169), the CDR2 sequence of LVARITGGGSTH (SEQ ID NO: 199), and the CDR3 sequence of ASMVNPIITAWGTIGVREIPDYD (SEQ ID NO: 231); or
   vi) the CDR1 sequence of RSIGSINVMG (SEQ ID NO: 179), the CDR2 sequence of RITGGGSTH (SEQ ID NO: 209), and the CDR3 sequence of MVNPIITAWGTIGVREIPDYDY (SEQ ID NO: 242);
h) VHH9:
   i) the CDR1 sequence of TYRMG (SEQ ID NO: 7), the CDR2 sequence of AISWSGGSTTYADPVKG (SEQ ID NO: 36), and the CDR3 sequence of DQRGY (SEQ ID NO: 67) or QRGY (SEQ ID NO: 271);
   ii) the CDR1 sequence of GRTFSTY (SEQ ID NO: 16), the CDR2 sequence of SWSGGS (SEQ ID NO: 46) or WSGG (SEQ ID NO: 266), and the CDR3 sequence of DQRGY (SEQ ID NO: 78) or RG (SEQ ID NO: 279);
   iii) the CDR1 sequence of GRTFSTYR (SEQ ID NO: 26), the CDR2 sequence of ISWSGGST (SEQ ID NO: 56), and the CDR3 sequence of NDQRGY (SEQ ID NO: 89);
   iv) the CDR1 sequence of GRTFSTYRMG (SEQ ID NO: 160), the CDR2 sequence of AISWSGGSTTYADPVKG (SEQ ID NO: 190), and the CDR3 sequence of QRGY (SEQ ID NO: 221);
   v) the CDR1 sequence of STYRMG (SEQ ID NO: 170), the CDR2 sequence of FVAAISWSGGSTT (SEQ ID NO: 200), and the CDR3 sequence of NDQRG (SEQ ID NO: 232); or
   vi) the CDR1 sequence of GRTFSTYRMG (SEQ ID NO: 180), the CDR2 sequence of AISWSGGSTT (SEQ ID NO: 210), and the CDR3 sequence of QRGY (SEQ ID NO: 243);
i) VHH10:
   i) the CDR1 sequence of RYAMG (SEQ ID NO: 8), the CDR2 sequence of AISWSGSSAGYGDSVKG (SEQ ID NO: 37), and the CDR3 sequence of DPFNQGY (SEQ ID NO: 68);
   ii) the CDR1 sequence of GFTFTRY (SEQ ID NO: 17), the CDR2 sequence of SWSGSS (SEQ ID NO: 47) or WSGS (SEQ ID NO: 267), and the CDR3 sequence of DPFNQGY (SEQ ID NO: 79) or PFNQG (SEQ ID NO: 280);
   iii) the CDR1 sequence of GFTFTRYA (SEQ ID NO: 27), the CDR2 sequence of ISWSGSSA (SEQ ID NO: 57), and the CDR3 sequence of AADPFNQGY (SEQ ID NO: 90);
   iv) the CDR1 sequence of GFTFTRYAMG (SEQ ID NO: 161), the CDR2 sequence of AISWSGSSAGYGDSVKG (SEQ ID NO: 191), and the CDR3 sequence of DPFNQGY (SEQ ID NO: 222);
   v) the CDR1 sequence of TRYAMG (SEQ ID NO: 171), the CDR2 sequence of FVAAISWSGSSAG (SEQ ID NO: 201), and the CDR3 sequence of AADPFNQG (SEQ ID NO: 233); or
   vi) the CDR1 sequence of GFTFTRYAMG (SEQ ID NO: 181), the CDR2 sequence of AISWSGSSAG (SEQ ID NO: 211), and the CDR3 sequence of DPFNQGY (SEQ ID NO: 244);
j) VHH11:
   i) the CDR1 sequence of FTTYRMG (SEQ ID NO: 258) or TYRMG (SEQ ID NO: 259), the CDR2 sequence of AIRWSGGRTLYADSVKG (SEQ ID NO: 38), and the CDR3 sequence of DLAEYSGTYSSPADSPAGYDY (SEQ ID NO: 69);
   ii) the CDR1 sequence of GRTFTTY (SEQ ID NO: 18), the CDR2 sequence of RWSGGR (SEQ ID NO: 48) or WSGG (SEQ ID NO: 268), and the CDR3 sequence of DLAEYSGTYSSPADSPAGYDY (SEQ ID NO: 80) or LAEYSGTYSSPADSPAGYD (SEQ ID NO: 281);
   iii) the CDR1 sequence of GRTFTTYR (SEQ ID NO: 28), the CDR2 sequence of IRWSGGRT (SEQ ID NO: 58), and the CDR3 sequence of AADLAEYSGTYSSPADSPAGYDY (SEQ ID NO: 91);
   iv) the CDR1 sequence of GRTFTTYRMG (SEQ ID NO: 162), the CDR2 sequence of AIRWSGGRTLYADSVKG (SEQ ID NO: 192), and the CDR3 sequence of DLAEYSGTYSSPADSPAGYDY (SEQ ID NO: 223);
   v) the CDR1 sequence of TTYRMG (SEQ ID NO: 172), the CDR2 sequence of FVAAIRWSGGRTL (SEQ ID NO: 202), and the CDR3 sequence of AADLAEYSGTYSSPADSPAGYD (SEQ ID NO: 234); or
   vi) the CDR1 sequence of GRTFTTYRMG (SEQ ID NO: 182), the CDR2 sequence of AIRWSGGRTL (SEQ ID NO: 212), and the CDR3 sequence of DLAEYSGTYSSPADSPAGYDY (SEQ ID NO: 245);
k) VHH12:
   i) the CDR1 sequence of FNTYAMG (SEQ ID NO: 9), the CDR2 sequence of SITWNGGSTSYADSVKG (SEQ ID NO: 39), and the CDR3 sequence of ARYYVSGTYFPANY (SEQ ID NO: 70);
   ii) the CDR1 sequence of GRTLSFNTY (SEQ ID NO: 19), the CDR2 sequence of TWNGGS (SEQ ID NO: 49) or WNGG (SEQ ID NO: 269), and the CDR3 sequence of ARYYVSGTYFPANY (SEQ ID NO: 81) or RYYVSGTYFPAN (SEQ ID NO: 282);
   iii) the CDR1 sequence of GRTLSFNTYA (SEQ ID NO: 29), the CDR2 sequence of ITWNGGST (SEQ ID NO: 59), and the CDR3 sequence of AAARYYVSGTYFPANY (SEQ ID NO: 92);
   iv) the CDR1 sequence of GRTLSFNTYAMG (SEQ ID NO: 163), the CDR2 sequence of SITWNGGSTSYADSVKG (SEQ ID NO: 193), and the CDR3 sequence of ARYYVSGTYFPANY (SEQ ID NO: 224);
   v) the CDR1 sequence of SFNTYAMG (SEQ ID NO: 173), the CDR2 sequence of FVASITWNGGSTS (SEQ ID NO: 203), and the CDR3 sequence of AAARYYVSGTYFPAN (SEQ ID NO: 235); or
   vi) the CDR1 sequence of GRTLSFNTYAMG (SEQ ID NO: 183), the CDR2 sequence of SITWNGGSTS (SEQ ID NO: 213), and the CDR3 sequence of ARYYVSGTYFPANY (SEQ ID NO: 246).

263. The VHH domain of any one of embodiments 252-262, wherein the VHH domain comprises a framework derived from the framework of any of the VHH domains comprising the sequences of

(SEQ ID NO: 93)
QVQLVESGGGLVQAGGSLKLACAAPGLTFSSYRMGWFRQAPGQEREFVAA
IDWNGRGTYYRYYADSVKGRSTISRDNAKNTMYLQMNSLKPEDTAVYYCA
AGSIDLNWYGGMDYWGQGTQVTVSS,
(SEQ ID NO: 94)
EVQVVESGGGLVQAGGSLKLACAAPGLTFSSYRMGWFRQAPGQEREFVAA
IDWNGRGTYYRYYADSVKGRSTISRDNAKNTVYLQMNSLKPEDTAVYYCA
ATTVLTDPRVLNEYATWGQGTQVTVSS,
(SEQ ID NO: 95)
QLQLVESGGGLVQPGGSLRLSCAASGSIFSINVMGWYRQAPGKQRELVAR
INGGGITHYAESVKGRFTISRDNAKNTVYLQMNSLKPEDTAAYYCKADVF
GSSGYVETYWGQGTQVTVSS,
(SEQ ID NO: 96)
EVQVVESGGGLVQAGGSLRLSCAVSGTSVSSNAMGWYRQAPGKQREWVGF
IDRIATTTIATSVKGRFAITRDNAKNTVYLQMSGLKPEDTAVYYCNHPLT
ARWGQGTQVTVSS,
(SEQ ID NO: 97)
QVQLVESGGGLVQAGGSLRLSCAASGRTFSSYAMGWFRQAPGKEREFVAA
ITWNGGTTYYADSVKGRFTISRDNAKNTVYLQMNSLKPEDTAVYYCAADP
FNQGYWGQGTQVTVSS,
(SEQ ID NO: 98)
EVQLVESGGGLVQAGGSLRLSCAVSGSSVSSDAMGWYRQAPGNQRAWVAF
ISGGGTTTYADSVKGRFTISRDNTKNTVYLHMNSLKPEDTAVYYCNHPLT
SRWGQGTQVTVSS,
(SEQ ID NO: 99)
EVQVVESGGGLVQAGGSLRLACVASRSIGSINVMGWYRQAPGKQRDLVAR
ITGGGSTHYAESVKGRFTISRDNAKNTVYLQMNSLEPEDTAVYYCASMVN
PIITAWGTIGVREIPDYDYWGQGTQVTVSS,
(SEQ ID NO: 100)
QVQLVESGGGLVQAGGSLRLSCAVSGRTFSTYRMGWFRQAPGKERSFVAA
ISWSGGSTTYADPVKGRFTISRDNAKNTVYLRMNSLKPEDTAVYYCNDQR
GYWGQGTLVTVSS,
(SEQ ID NO: 101)
EVQVVESGGGLVQAGGSLRLSCAASGFTFTRYAMGWFRQAPGKERSFVAA
ISWSGSSAGYGDSVKGRFTISRDNAKNTLYLQMNSLKPEDTAVYYCAADP
FNQGYWGQGTQVTVSS,
(SEQ ID NO: 102)
EVQVVESGGGLVQAGGSLRLSCAASGRTFTTYRMGWFRQAPGKEREFVAA
IRWSGGRTLYADSVKGRFTISRDNAKNTAYLQMNNLRPEDTAVYYCAADL
AEYSGTYSSPADSPAGYDYWGQGTQVTVSS,
or
(SEQ ID NO: 103)
QVQLVETGGGLVQAGDSLRLSCAASGRTLSFNTYAMGWFRQAPGKEREFV
ASITWNGGSTSYADSVKGRFTITRDNAKNTATLRMNSLQPDDTAVYYCAA
ARYYVSGTYFPANYWGQGTQVTVSS.

264. The VHH domain of any one of embodiments 252-262, wherein the VHH domain comprises a framework comprising sequence having at least 75%, 80%, 85%, 90%, 95%, 96%, 97%, 98%, 99%, or 100% sequence identity with the sequence of

(SEQ ID NO: 93)
QVQLVESGGGLVQAGGSLKLACAAPGLTFSSYRMGWFRQAPGQEREFVA
AIDWNGRGTYYRYYADSVKGRSTISRDNAKNTMYLQMNSLKPEDTAVYY
CAAGSIDLNWYGGMDYWGQGTQVTVSS,
(SEQ ID NO: 94)
EVQVVESGGGLVQAGGSLKLACAAPGLTFSSYRMGWFRQAPGQEREFVA
AIDWNGRGTYYRYYADSVKGRSTISRDNAKNTVYLQMNSLKPEDTAVYY
CAATTVLTDPRVLNEYATWGQGTQVTVSS,
(SEQ ID NO: 95)
QLQLVESGGGLVQPGGSLRLSCAASGSIFSINVMGWYRQAPGKQRELVA
RINGGGITHYAESVKGRFTISRDNAKNTVYLQMNSLKPEDTAAYYCKAD
VFGSSGYVETYWGQGTQVTVSS,
(SEQ ID NO: 96)
EVQVVESGGGLVQAGGSLRLSCAVSGTSVSSNAMGWYRQAPGKQREWVG
FIDRIATTTIATSVKGRFAITRDNAKNTVYLQMSGLKPEDTAVYYCNHP
LTARWGQGTQVTVSS,
(SEQ ID NO: 97)
QVQLVESGGGLVQAGGSLRLSCAASGRTFSSYAMGWFRQAPGKEREFVA
AITWNGGTTYYADSVKGRFTISRDNAKNTVYLQMNSLKPEDTAVYYCAA
DPFNQGYWGQGTQVTVSS,
(SEQ ID NO: 98)
EVQLVESGGGLVQAGGSLRLSCAVSGSSVSSDAMGWYRQAPGNQRAWVA
FISGGGTTTYADSVKGRFTISRDNTKNTVYLHMNSLKPEDTAVYYCNHP
LTSRWGQGTQVTVSS,
(SEQ ID NO: 99)
EVQVVESGGGLVQAGGSLRLACVASRSIGSINVMGWYRQAPGKQRDLVA
RITGGGSTHYAESVKGRFTISRDNAKNTVYLQMNSLEPEDTAVYYCASM
VNPIITAWGTIGVREIPDYDYWGQGTQVTVSS,
(SEQ ID NO: 100)
QVQLVESGGGLVQAGGSLRLSCAVSGRTFSTYRMGWFRQAPGKERSFVA
AISWSGGSTTYADPVKGRFTISRDNAKNTVYLRMNSLKPEDTAVYYCND
QRGYWGQGTLVTVSS,
(SEQ ID NO: 101)
EVQVVESGGGLVQAGGSLRLSCAASGFTFTRYAMGWFRQAPGKERSFVA
AISWSGSSAGYGDSVKGRFTISRDNAKNTLYLQMNSLKPEDTAVYYCAA
DPFNQGYWGQGTQVTVSS,
(SEQ ID NO: 102)
EVQVVESGGGLVQAGGSLRLSCAASGRTFTTYRMGWFRQAPGKEREFVA
AIRWSGGRTLYADSVKGRFTISRDNAKNTAYLQMNNLRPEDTAVYYCAA
DLAEYSGTYSSPADSPAGYDYWGQGTQVTVSS,
or
(SEQ ID NO: 103)
QVQLVETGGGLVQAGDSLRLSCAASGRTLSFNTYAMGWFRQAPGKEREF
VASITWNGGSTSYADSVKGRFTITRDNAKNTATLRMNSLQPDDTAVYYC
AAARYYVSGTYFPANYWGQGTQVTVSS.

265. The domain of any one of embodiments 252 to 264, wherein the VHH domain is comprised of a sequence having at least 75%, 80%, 85%, 90%, 95%, 96%, 97%, 98%, 99%, or 100% sequence identity with the sequence of

(SEQ ID NO: 93)
QVQLVESGGGLVQAGGSLKLACAAPGLTFSSYRMGWFRQAPGQEREFVA
AIDWNGRGTYYRYYADSVKGRSTISRDNAKNTMYLQMNSLKPEDTAVYY
CAAGSIDLNWYGGMDYWGQGTQVTVSS,
(SEQ ID NO: 94)
EVQVVESGGGLVQAGGSLKLACAAPGLTFSSYRMGWFRQAPGQEREFVA
AIDWNGRGTYYRYYADSVKGRSTISRDNAKNTVYLQMNSLKPEDTAVYY
CAATTVLTDPRVLNEYATWGQGTQVTVSS,
(SEQ ID NO: 95)
QLQLVESGGGLVQPGGSLRLSCAASGSIFSINVMGWYRQAPGKQRELVA
RINGGGITHYAESVKGRFTISRDNAKNTVYLQMNSLKPEDTAAYYCKAD
VFGSSGYVETYWGQGTQVTVSS,
(SEQ ID NO: 96)
EVQVVESGGGLVQAGGSLRLSCAVSGTSVSSNAMGWYRQAPGKQREWVG
FIDRIATTTIATSVKGRFAITRDNAKNTVYLQMSGLKPEDTAVYYCNHP
LTARWGQGTQVTVSS,
(SEQ ID NO: 97)
QVQLVESGGGLVQAGGSLRLSCAASGRTFSSYAMGWFRQAPGKEREFVA
AITWNGGTTYYADSVKGRFTISRDNAKNTVYLQMNSLKPEDTAVYYCAA
DPFNQGYWGQGTQVTVSS,
(SEQ ID NO: 98)
EVQLVESGGGLVQAGGSLRLSCAVSGSSVSSDAMGWYRQAPGNQRAWVA
FISGGGTTTYADSVKGRFTISRDNTKNTVYLHMNSLKPEDTAVYYCNHP
LTSRWGQGTQVTVSS,
(SEQ ID NO: 99)
EVQVVESGGGLVQAGGSLRLACVASRSIGSINVMGWYRQAPGKQRDLVA
RITGGGSTHYAESVKGRFTISRDNAKNTVYLQMNSLEPEDTAVYYCASM
VNPIITAWGTIGVREIPDYDYWGQGTQVTVSS,
(SEQ ID NO: 100)
QVQLVESGGGLVQAGGSLRLSCAVSGRTFSTYRMGWFRQAPGKERSFVA
AISWSGGSTTYADPVKGRFTISRDNAKNTVYLRMNSLKPEDTAVYYCND
QRGYWGQGTLVTVSS,
(SEQ ID NO: 101)
EVQVVESGGGLVQAGGSLRLSCAASGFTFTRYAMGWFRQAPGKERSFVA
AISWSGSSAGYGDSVKGRFTISRDNAKNTLYLQMNSLKPEDTAVYYCAA
DPFNQGYWGQGTQVTVSS,
(SEQ ID NO: 102)
EVQVVESGGGLVQAGGSLRLSCAASGRTFTTYRMGWFRQAPGKEREFVA
AIRWSGGRTLYADSVKGRFTISRDNAKNTAYLQMNNLRPEDTAVYYCAA
DLAEYSGTYSSPADSPAGYDYWGQGTQVTVSS,
or
(SEQ ID NO: 103)
QVQLVETGGGLVQAGDSLRLSCAASGRTLSFNTYAMGWFRQAPGKEREF
VASITWNGGSTSYADSVKGRFTITRDNAKNTATLRMNSLQPDDTAVYYC
AAARYYVSGTYFPANYWGQGTQVTVSS.

266. A therapeutic molecule comprising the VHH domain of any of embodiments 252 to 265 and an agent.

267. The therapeutic molecule of embodiment 266, wherein the agent is an antibody or fragment thereof, a peptide, a vaccine, a small molecule, a polynucleotide, a radioisotope, a toxin, an enzyme, an anticoagulant, a hormone, a cytokine, an anti-inflammatory molecule, an RNAi, an mRNA, a self-replicating RNA, an antibiotic, or an antibody-antibiotic conjugate.

268. The therapeutic molecule of embodiment 267, wherein the agent is an antibiotic, an antibody or fragment thereof, a peptide or a vaccine.

269. The therapeutic molecule of any one of embodiments 266 to 268, wherein the VHH domain is genetically fused or chemically conjugated to the agent.

270. The therapeutic molecule of embodiment 269, further comprising a linker between the VHH domain and the agent.

271. The therapeutic molecule of embodiment 270, wherein the linker is a polypeptide.

272. The therapeutic molecule of embodiment 271, wherein the linker is a flexible linker comprising a sequence selected from the group consisting of EPKTPKPQPQPQLQPQPNPTTESKSPK (SEQ ID NO: 130), (EAAAK)n (SEQ ID NO: 147), (GGGGS)n (SEQ ID NO: 148) and (GGGS)n (SEQ ID NO: 149), wherein n is an integer from 252 to 271.

273. The therapeutic molecule of any one of embodiments 266 to 272, wherein the VHH domain is chemically-conjugated to the agent.

274. The therapeutic molecule of any one of embodiments 266 to 272, wherein the VHH domain is non-covalently bound to the agent.

275. An isolated nucleic acid molecule encoding the VHH domain of any of embodiments 252 to 265.

276. An isolated nucleic acid molecule encoding the VHH domain having a sequence with at least 85%, 90%, 95%, 96%, 97%, 98%, 99% or 100% sequence identity to the sequence of QVQLVESGGGLVQAGGSLKLACAAPGLTFSSYRMGWFRQAPGQEREFVAAIDWNGRG TYYRYYADSVKGRSTISRDNAKNTMYLQMNSLKPEDTAVYYCAAGSIDLNWYGGMD YWGQGTQVTVSS (SEQ ID NO: 93), EVQVVESGGGLVQAGGSLKLACAAPGLTFSSYRMGWFRQAPGQEREFVAAIDWNGRG TYYRYYADSVKGRSTISRDNAKNTVYLQMNSLKPEDTAVYYCAATTVLTDPRVLNEYA TWGQGTQVTVSS (SEQ ID NO: 94), QLQLVESGGGLVQPGGSLRL SCAASGSIFSINVMGWYRQAPGKQRELVARINGGGITHY AESVKGRFTISRDNAKNTVYLQMNSLKPEDTAAYYCKADVFGSSGYVETYWGQGTQV TVSS (SEQ ID NO: 95), EVQVVESGGGLVQAGGSLRLSCAVSGTSVSSNAMGWYRQAPGKQREWVGFIDRIATTT IATSVKGRFAITRDNAKNTVYLQMSGLKPEDTAVYYCNHPLTARWGQGTQVTVSS (SEQ ID NO: 96), QVQLVESGGGLVQAGGSLRL SCAASGRTFSSYAMGWFRQAPGKEREFVAAITWNGGTT YYADSVKGRFTISRDNAKNTVYLQMNSLKPEDTAVYYCAADPFNQGYWGQGTQVTVS S (SEQ ID NO: 97), EVQLVESGGGLVQAGGSLRLSCAVSGSSVSSDAMGWYRQAPGNQRAWVAFISGGGTT TYADSVKGRFTISRDNTKNTVYLHMNSLKPEDTAVYYCNHPLTSRWGQGTQVTVSS (SEQ ID NO: 98), EVQVVESGGGLVQAGGSLRLACVASRSIGSINVMGWYRQAPGKQRDLVARITGGGSTH YAESVKGRFTISRDNAKNTVYLQMNSLEPEDTAVYYCASMVNPIITAWGTIGVREIPDY DYWGQGTQVTVSS (SEQ ID NO: 99), QVQLVESGGGLVQAGGSLRLSCAVSGRTFSTYRMGWFRQAPGKERSFVAAISWSGGST TYADPVKGRFTISRDNAKNTVYLRMNSLKPEDTAVYYCNDQRGYWGQGTLVTVSS (SEQ ID NO: 100), EVQVVESGGGLVQAGGSLRLSCAASGFTFTRYAMGWFRQAPGKERSFVAAISWSGSSA GYGDSVKGRF TISRDNAKNTLYLQMNSLKPEDTAVYYCAADPFNQGYWGQGTQVTVS S (SEQ ID NO: 101), EVQVVESGGGLVQAGGSLRL SCAASGRTFTTYRMGWFRQAPGKEREFVAAIRWSGGRT LYADSVKGRF TISRDNAKNTAYLQMNNLRPED TAVYYCAADLAEYSGTYSSPADSPAG YDYWGQGTQVTVSS (SEQ ID NO: 102), or QVQLVETGGGLVQAGDSLRLSCAASGRTLSFNTYAMGWFRQAPGKEREFVASITWNG GSTSYADSVKGRFTITRDNAKNTATLRMNSLQPDDTAVYYCAAARYYVSGTYFPANY WGQGTQVTVSS (SEQ ID NO: 103), optionally wherein the nucleic acid molecule comprises a sequence with at least 85%, 90%, 95%, 96%, 97%, 98%, 99% or 100% sequence identity to the polynucleotide sequence of any one of SEQ ID NOS: 133-142.

277. A vector comprising the nucleic acid molecule of embodiment 275 or embodiment 276.

278. A cell expressing the nucleic acid molecule of embodiment 275 or embodiment 276.

279. A pharmaceutical composition comprising the VHH domain of any of embodiments 252 to 265, including an effective amount of the VHH domain, and a pharmaceutically acceptable excipient.

280. A pharmaceutical composition, comprising the therapeutic molecule of any of embodiments 266 to 274, including an effective amount of the VHH, and a pharmaceutically acceptable carrier.

281. A method of increasing the rate of pIgR-mediated transcytosis (e.g., forward transcytosis or reverse transcytosis) across an epithelial cell comprising contacting the cell with the VHH domain of any of embodiments 252 to 265 or the therapeutic molecule of any of embodiments 266 to 274.

282. The method of embodiment 281, wherein the method does not inhibit pIgR-mediated transcytosis of IgA.

283. The method of embodiment 282, wherein the VHH domain or the therapeutic molecule comprises a CDR1 sequence of SNAMG (SEQ ID NO: 3), INVMG (SEQ ID NO: 6), TYRMG (SEQ ID NO: 7), RYAMG (SEQ ID NO: 8), FTTYRMG (SEQ ID NO: 258), TYRMG (SEQ ID NO: 259), FNTYAMG (SEQ ID NO: 9), GTSVSSN (SEQ ID NO: 12), GRTFSSY (SEQ ID NO: 13), RSIGSIN (SEQ ID NO: 15), GRTFSTY (SEQ ID NO: 16), GFTFTRY (SEQ ID NO: 17), GRTFTTY (SEQ ID NO: 18), GRTLSFNTY (SEQ ID NO: 19), GTSVSSNA (SEQ ID NO: 22), RSIGSINV (SEQ ID NO: 25), GRTFSTYR (SEQ ID NO: 26), GFTFTRYA (SEQ ID NO: 27), GRTFTTYR (SEQ ID NO: 28), GRTLSFNTYA (SEQ ID NO: 29), GTSVSSNAMG (SEQ ID NO: 156), RSIGSINVMG (SEQ ID NO: 159), GRTFSTYRMG (SEQ ID NO: 160), GFTFTRYAMG (SEQ ID NO: 161), GRTFTTYRMG (SEQ ID NO: 162), GRTLSFNTYAMG (SEQ ID NO: 163), SSNAMG (SEQ ID NO: 166), SINVMG (SEQ ID NO: 169), STYRMG (SEQ ID NO: 170), TRYAMG (SEQ ID NO: 171), TTYRMG (SEQ ID NO: 172), SFNTYAMG (SEQ ID NO: 173), GTSVSSNAMG (SEQ ID NO: 176), RSIGSINVMG (SEQ ID NO: 179), GRTFSTYRMG (SEQ ID NO: 180), GFTFTRYAMG (SEQ ID NO: 181), GRTFTTYRMG (SEQ ID NO: 182), or GRTLSFNTYAMG (SEQ ID NO: 183).

284. The method of embodiment 282 or embodiment 283, wherein the VHH domain comprises a CDR2 sequence of FIDRIATTTIATSVKG (SEQ ID NO: 32), RITGGGSTHYAESVKG (SEQ ID NO: 35), AISWSGGSTTYADPVKG (SEQ ID NO: 36), AISWSGSSAGYGDSVKG (SEQ ID NO: 37), AIRWSGGRTLYADSVKG (SEQ ID NO: 38), SITWNGGSTSYADSVKG (SEQ ID NO: 39), DRIAT (SEQ ID NO: 42), RIA (SEQ ID NO: 262), TGGGS (SEQ ID NO: 45), GGG (SEQ ID NO: 265), SWSGGS (SEQ ID NO: 46), WSGG (SEQ ID NO: 266), SWSGSS (SEQ ID NO: 47), WSGS (SEQ ID NO: 267), RWSGGR (SEQ ID NO: 48), WSGG (SEQ ID NO: 268), TWNGGS (SEQ ID NO: 49), WNGG (SEQ ID NO: 269), IDRIATT (SEQ ID NO: 52), ITGGGST (SEQ ID NO: 55), ISWSGGST (SEQ ID NO: 56), ISWSGSSA (SEQ ID NO: 57), IRWSGGRT (SEQ ID NO: 58), ITWNGGST (SEQ ID NO: 59), FIDRIATTTIATSVKG (SEQ ID NO: 186), RITGGGSTHYAESVKG (SEQ ID NO: 189), AISWSGGSTTYADPVKG (SEQ ID NO: 190), AISWSGSSAGYGDSVKG (SEQ ID NO: 191), AIRWSGGRTLYADSVKG (SEQ ID NO: 192), SITWNGGSTSYADSVKG (SEQ ID NO: 193), WVGFIDRIATTT (SEQ ID NO: 196), LVARITGGGSTH (SEQ ID NO: 199), FVAAISWSGGSTT (SEQ ID NO: 200), FVAAISWSGSSAG (SEQ ID NO: 201), FVAAIRWSGGRTL (SEQ ID NO: 202), FVASITWNGGSTS (SEQ ID NO: 203), FIDRIATTT (SEQ ID NO: 206), RITGGGSTH (SEQ ID NO: 209), AISWSGGSTT (SEQ ID NO: 210), AISWSGSSAG (SEQ ID NO: 211), AIRWSGGRTL (SEQ ID NO: 212), or SITWNGGSTS (SEQ ID NO: 213).

285. The method of any one of embodiments 282 to 284, wherein the VHH domain comprises a CDR3 sequence of PLTAR (SEQ ID NO: 63), MVNPIITAWGTIGVREIPDYDY (SEQ ID NO: 66), DQRGY (SEQ ID NO: 67), QRGY (SEQ ID NO: 271), DPFNQGY (SEQ ID NO: 68), DLAEYSGTYSSPADSPAGYDY (SEQ ID NO: 69), ARYYVSGTYFPANY (SEQ ID NO: 70), PLTAR (SEQ ID NO: 74), LTA (SEQ ID NO: 275), MVNPIITAWGTIGVREIPDYDY (SEQ ID NO: 77), VNPIITAWGTIGVREIPDYD (SEQ ID NO: 278), DQRGY (SEQ ID NO: 78), RG (SEQ ID NO: 279), DPFNQGY (SEQ ID NO: 79), PFNQG (SEQ ID NO: 280), DLAEYSGTYSSPADSPAGYDY (SEQ ID NO: 80), LAEYSGTYSSPADSPAGYD (SEQ ID NO: 281), ARYYVSGTYFPANY (SEQ ID NO: 81), RYYVSGTYFPAN (SEQ ID NO: 282), NHPLTAR (SEQ ID NO: 85), ASMVNPIITAWGTIGVREIPDYDY (SEQ ID NO: 88), NDQRGY (SEQ ID NO: 89), AADPFNQGY (SEQ ID NO: 90), AADLAEYSGTYSSPADSPAGYDY (SEQ ID NO: 91), AAARYYVSGTYFPANY (SEQ ID NO: 92), PLTAR (SEQ ID NO: 217), MVNPIITAWGTIGVREIPDYDY (SEQ ID NO: 220), QRGY (SEQ ID NO: 221), DPFNQGY (SEQ ID NO: 222), DLAEYSGTYSSPADSPAGYDY (SEQ ID NO: 223), ARYYVSGTYFPANY (SEQ ID NO: 224), NHPLTA (SEQ ID NO: 228), ASMVNPIITAWGTIGVREIPDYD (SEQ ID NO: 231), NDQRG (SEQ ID NO: 232), AADPFNQG (SEQ ID NO: 233), AADLAEYSGTYSSPADSPAGYD (SEQ ID NO: 234), AAARYYVSGTYFPAN (SEQ ID NO: 235), PLTAR (SEQ ID NO: 239), MVNPIITAWGTIGVREIPDYDY (SEQ ID NO: 242), QRGY (SEQ ID NO: 243), DPFNQGY (SEQ ID NO: 244), DLAEYSGTYSSPADSPAGYDY (SEQ ID NO: 245), or ARYYVSGTYFPANY (SEQ ID NO: 246), including wherein optionally the VHH domain comprises a CDR1 sequence, a CDR2 sequence, and a CDR3 sequence of the VHH domain selected from the group consisting of:
a) VHH1:
   i) the CDR1 sequence of SYRMG (SEQ ID NO: 1), the CDR2 sequence of AIDWNGRGTYYRYYADSVKG (SEQ ID NO: 30), and the CDR3 sequence of GSIDLNWYGGMDY (SEQ ID NO: 60);
   ii) the CDR1 sequence of GLTFSSY (SEQ ID NO: 10), the CDR2 sequence of DWNGRGTYY (SEQ ID NO: 40) or WNGRGTY (SEQ ID NO: 260), and the CDR3 sequence of GSIDLNWYGGMDY (SEQ ID NO: 71) or SIDLNWYGGMD (SEQ ID NO: 272);
   iii) the CDR1 sequence of GLTFSSYR (SEQ ID NO: 20), the CDR2 sequence of IDWNGRGTYY (SEQ ID NO: 50) or IDWNGRGTYYR (SEQ ID NO: 270), and the CDR3 sequence of CAAGSIDLNWYGGMDY (SEQ ID NO: 82) or AAGSIDLNWYGGMDY (SEQ ID NO: 283);
   iv) the CDR1 sequence of GLTFSSYRMG (SEQ ID NO: 154), the CDR 2 sequence of AIDWNGRGTYYRYYADSVKG (SEQ ID NO: 184), and the CDR3 sequence of GSIDLNWYGGMDY (SEQ ID NO: 214);
   v) the CDR1 sequence of SSYRMG (SEQ ID NO: 164), the CDR2 sequence of FVAAIDWNGRGTYYRY (SEQ ID NO: 194), and the CDR3 sequence of AAGSIDLNWYGGMD (SEQ ID NO: 225); or
   vi) the CDR1 sequence of GLTFSSYRMG (SEQ ID NO: 174), the CDR2 sequence of AIDWNGRGTYYRY (SEQ ID NO: 204), and the CDR3 sequence of GSIDLNWYGGMDY (SEQ ID NO: 236);
b) VHH2:
   i) the CDR1 sequence of SYRMG (SEQ ID NO: 1), the CDR2 sequence of AIDWNGRGTYYRYYADSVKG (SEQ ID NO: 30), and the CDR3 sequence of TTVLTDPRVLNEYAT (SEQ ID NO: 61);
   ii) the CDR1 sequence of GLTFSSY (SEQ ID NO: 10), the CDR2 sequence of DWNGRGTYY (SEQ ID NO: 40) or WNGRGTY (SEQ ID NO: 260), and the CDR3 sequence of TTVLTDPRVLNEYAT (SEQ ID NO: 72) or TVLTDPRVLNEYA (SEQ ID NO: 273);
   iii) the CDR1 sequence of GLTFSSYR (SEQ ID NO: 20), the CDR2 sequence of IDWNGRGTYY (SEQ ID NO: 50) or IDWNGRGTYYR (SEQ ID NO: 270), and the CDR3 sequence of CAATTVLTDPRVLNEYAT (SEQ ID NO: 83) or AATTVLTDPRVLNEYAT (SEQ ID NO: 284);
   iv) the CDR1 sequence of GLTFSSYRMG (SEQ ID NO: 154), the CDR2 sequence of AIDWNGRGTYYRYYADSVKG (SEQ ID NO: 184), and the CDR3 sequence of TTVLTDPRVLNEYAT (SEQ ID NO: 215);
   v) the CDR1 sequence of SSYRMG (SEQ ID NO: 164), the CDR2 sequence of FVAAIDWNGRGTYYRY (SEQ ID NO: 194), and the CDR3 sequence of AATTVLTDPRVLNEYA (SEQ ID NO: 226); or
   vi) the CDR1 sequence of GLTFSSYRMG (SEQ ID NO: 174), the CDR2 sequence of AIDWNGRGTYYRY (SEQ ID NO: 204), and the CDR3 sequence of TTVLTDPRVLNEYAT (SEQ ID NO: 237);
c) VHH3:
   i) the CDR1 sequence of INVMG (SEQ ID NO: 2), the CDR2 sequence of RINGGGITHYAESVKG (SEQ ID NO: 31), and the CDR3 sequence of DVFGSSGYVETY (SEQ ID NO: 62);
   ii) the CDR1 sequence of GSIFSIN (SEQ ID NO: 11), the CDR2 sequence of NGGGI (SEQ ID NO: 41) or GGG (SEQ ID NO: 261), and the CDR3 sequence of DVFGSSGYVETY (SEQ ID NO: 73) or VFGSSGYVET (SEQ ID NO: 274);
   iii) the CDR1 sequence of GSIFSINV (SEQ ID NO: 21), the CDR2 sequence of INGGGIT (SEQ ID NO: 51), and the CDR3 sequence of KADVFGSSGYVETY (SEQ ID NO: 84);
   iv) the CDR1 sequence of GSIFSINVMG (SEQ ID NO: 155), the CDR2 sequence of RINGGGITHYAESVKG (SEQ ID NO: 185), and the CDR3 sequence of DVFGSSGYVETY (SEQ ID NO: 216);
   v) the CDR1 sequence of SINVMG (SEQ ID NO: 165), the CDR2 sequence of LVARINGGGITH (SEQ ID NO: 195), and the CDR3 sequence of KADVFGSSGYVET (SEQ ID NO: 227); or
   vi) the CDR1 sequence of GSIFSINVMG (SEQ ID NO: 175), the CDR2 sequence of RINGGGITH (SEQ ID NO: 205), and the CDR3 sequence of DVFGSSGYVETY (SEQ ID NO: 238);
d) VHH4:
   i) the CDR1 sequence of SNAMG (SEQ ID NO: 3), the CDR2 sequence of FIDRIATTTIATSVKG (SEQ ID NO: 32), and the CDR3 sequence of PLTAR (SEQ ID NO: 63);
   ii) the CDR1 sequence of GTSVSSN (SEQ ID NO: 12), the CDR2 sequence of DRIAT (SEQ ID NO: 42) or RIA (SEQ ID NO: 262), and the CDR3 sequence of PLTAR (SEQ ID NO: 74) or LTA (SEQ ID NO: 275);
   iii) the CDR1 sequence of GTSVSSNA (SEQ ID NO: 22), the CDR2 sequence of IDRIATT (SEQ ID NO: 52), and the CDR3 sequence of NHPLTAR (SEQ ID NO: 85);
   iv) the CDR1 sequence of GTSVSSNAMG (SEQ ID NO: 156), the CDR2 sequence of FIDRIATTTIATSVKG (SEQ ID NO: 186), and the CDR3 sequence of PLTAR (SEQ ID NO: 217);
   v) the CDR1 sequence of SSNAMG (SEQ ID NO: 166), the CDR2 sequence of WVGFIDRIATTT (SEQ ID NO: 196), and the CDR3 sequence of NHPLTA (SEQ ID NO: 228); or
   vi) the CDR1 sequence of GTSVSSNAMG (SEQ ID NO: 176), the CDR2 sequence of FIDRIATTT (SEQ ID NO: 206), and the CDR3 sequence of PLTAR (SEQ ID NO: 239);
e) VHH5:
   i) the CDR1 sequence of SYAMG (SEQ ID NO: 4), the CDR2 sequence of AITWNGGTTYYADSVKG (SEQ ID NO: 33), and the CDR3 sequence of DPFNQGY (SEQ ID NO: 64);
   ii) the CDR1 sequence of GRTFSSY (SEQ ID NO: 13), the CDR2 sequence of TWNGGT (SEQ ID NO: 43) or WNGG (SEQ ID NO: 263), and the CDR3 sequence of DPFNQGY (SEQ ID NO: 75) or PFNQG (SEQ ID NO: 276);
   iii) the CDR1 sequence of GRTFSSYA (SEQ ID NO: 23), the CDR2 sequence of ITWNGGTT (SEQ ID NO: 53), and the CDR3 sequence of AADPFNQGY (SEQ ID NO: 86);
   iv) the CDR1 sequence of GRTFSSYAMG (SEQ ID NO: 157), the CDR2 sequence of AITWNGGTTYYADSVKG (SEQ ID NO: 187), and the CDR3 sequence of DPFNQGY (SEQ ID NO: 218);
   v) the CDR1 sequence of SSYAMG (SEQ ID NO: 167), the CDR2 sequence of FVAAITWNGGTTY (SEQ ID NO: 197), and the CDR3 sequence of AADPFNQG (SEQ ID NO: 229); or
   vi) the CDR1 sequence of GRTFSSYAMG (SEQ ID NO: 177), the CDR2 sequence of AITWNGGTTY (SEQ ID NO: 207), and the CDR3 sequence of DPFNQGY (SEQ ID NO: 240);
f) VHH6:
   i) the CDR1 sequence of SDAMG (SEQ ID NO: 5), the CDR2 sequence of FISGGGTTTYADSVKG (SEQ ID NO: 34), and the CDR3 sequence of PLTSR (SEQ ID NO: 65);
   ii) the CDR1 sequence of GSSVSSD (SEQ ID NO: 14), the CDR2 sequence of SGGGT (SEQ ID NO: 44) or GGG (SEQ ID NO: 264), and the CDR3 sequence of PLTSR (SEQ ID NO: 76) or LTS (SEQ ID NO: 277);
   iii) the CDR1 sequence of GSSVSSDA (SEQ ID NO: 24), the CDR2 sequence of ISGGGTT (SEQ ID NO: 54), and the CDR3 sequence of NHPLTSR (SEQ ID NO: 87);
   iv) the CDR1 sequence of GSSVSSDAMG (SEQ ID NO: 158), the CDR2 sequence of FISGGGTTTYADSVKG (SEQ ID NO: 188), and the CDR3 sequence of PLTSR (SEQ ID NO: 219);
   v) the CDR1 sequence of SSDAMG (SEQ ID NO: 168), the CDR2 sequence of WVAFISGGGTTT (SEQ ID NO: 198), and the CDR3 sequence of NHPLTS (SEQ ID NO: 230); or
   vi) the CDR1 sequence of GSSVSSDAMG (SEQ ID NO: 178), the CDR2 sequence of FISGGGTTT (SEQ ID NO: 208), and the CDR3 sequence of PLTSR (SEQ ID NO: 241);
g) VHH7:
   i) the CDR1 sequence of INVMG (SEQ ID NO: 6), the CDR2 sequence of RITGGGSTHYAESVKG (SEQ ID NO: 35), and the CDR3 sequence of MVNPIITAWGTIGVREIPDYDY (SEQ ID NO: 66);
   ii) the CDR1 sequence of RSIGSIN (SEQ ID NO: 15), the CDR2 sequence of TGGGS (SEQ ID NO: 45) or GGG (SEQ ID NO: 265), and the CDR3 sequence of MVNPIITAWGTIGVREIPDYDY (SEQ ID NO: 77) or VNPIITAWGTIGVREIPDYD (SEQ ID NO: 278);
   iii) the CDR1 sequence of RSIGSINV (SEQ ID NO: 25), the CDR2 sequence of ITGGGST (SEQ ID NO: 55), and the CDR3 sequence of ASMVNPIITAWGTIGVREIPDYDY (SEQ ID NO: 88);
   iv) the CDR1 sequence of RSIGSINVMG (SEQ ID NO: 159), the CDR2 sequence of RITGGGSTHYAESVKG (SEQ ID NO: 189), and the CDR3 sequence of MVNPIITAWGTIGVREIPDYDY (SEQ ID NO: 220);
   v) the CDR1 sequence of SINVMG (SEQ ID NO: 169), the CDR2 sequence of LVARITGGGSTH (SEQ ID NO: 199), and the CDR3 sequence of ASMVNPIITAWGTIGVREIPDYD (SEQ ID NO: 231); or
   vi) the CDR1 sequence of RSIGSINVMG (SEQ ID NO: 179), the CDR2 sequence of RITGGGSTH (SEQ ID NO: 209), and the CDR3 sequence of MVNPIITAWGTIGVREIPDYDY (SEQ ID NO: 242);
h) VHH9:
   i) the CDR1 sequence of TYRMG (SEQ ID NO: 7), the CDR2 sequence of AISWSGGSTTYADPVKG (SEQ ID NO: 36), and the CDR3 sequence of DQRGY (SEQ ID NO: 67) or QRGY (SEQ ID NO: 271);
   ii) the CDR1 sequence of GRTFSTY (SEQ ID NO: 16), the CDR2 sequence of SWSGGS (SEQ ID NO: 46) or WSGG (SEQ ID NO: 266), and the CDR3 sequence of DQRGY (SEQ ID NO: 78) or RG (SEQ ID NO: 279);
   iii) the CDR1 sequence of GRTFSTYR (SEQ ID NO: 26), the CDR2 sequence of ISWSGGST (SEQ ID NO: 56), and the CDR3 sequence of NDQRGY (SEQ ID NO: 89);
   iv) the CDR1 sequence of GRTFSTYRMG (SEQ ID NO: 160), the CDR2 sequence of AISWSGGSTTYADPVKG (SEQ ID NO: 190), and the CDR3 sequence of QRGY (SEQ ID NO: 221);
   v) the CDR1 sequence of STYRMG (SEQ ID NO: 170), the CDR2 sequence of FVAAISWSGGSTT (SEQ ID NO: 200), and the CDR3 sequence of NDQRG (SEQ ID NO: 232); or
   vi) the CDR1 sequence of GRTFSTYRMG (SEQ ID NO: 180), the CDR2 sequence of AISWSGGSTT (SEQ ID NO: 210), and the CDR3 sequence of QRGY (SEQ ID NO: 243);
i) VHH10:
   i) the CDR1 sequence of RYAMG (SEQ ID NO: 8), the CDR2 sequence of AISWSGSSAGYGDSVKG (SEQ ID NO: 37), and the CDR3 sequence of DPFNQGY (SEQ ID NO: 68);
   ii) the CDR1 sequence of GFTFTRY (SEQ ID NO: 17), the CDR2 sequence of SWSGSS (SEQ ID NO: 47) or WSGS (SEQ ID NO: 267), and the CDR3 sequence of DPFNQGY (SEQ ID NO: 79) or PFNQG (SEQ ID NO: 280);
   iii) the CDR1 sequence of GFTFTRYA (SEQ ID NO: 27), the CDR2 sequence of ISWSGSSA (SEQ ID NO: 57), and the CDR3 sequence of AADPFNQGY (SEQ ID NO: 90);
   iv) the CDR1 sequence of GFTFTRYAMG (SEQ ID NO: 161), the CDR2 sequence of AISWSGSSAGYGDSVKG (SEQ ID NO: 191), and the CDR3 sequence of DPFNQGY (SEQ ID NO: 222);
   v) the CDR1 sequence of TRYAMG (SEQ ID NO: 171), the CDR2 sequence of FVAAISWSGSSAG (SEQ ID NO: 201), and the CDR3 sequence of AADPFNQG (SEQ ID NO: 233); or
   vi) the CDR1 sequence of GFTFTRYAMG (SEQ ID NO: 181), the CDR2 sequence of AISWSGSSAG (SEQ ID NO: 211), and the CDR3 sequence of DPFNQGY (SEQ ID NO: 244);
j) VHH11:
   i) the CDR1 sequence of FTTYRMG (SEQ ID NO: 258) or TYRMG (SEQ ID NO: 259), the CDR2 sequence of AIRWSGGRTLYADSVKG (SEQ ID NO: 38), and the CDR3 sequence of DLAEYSGTYSSPADSPAGYDY (SEQ ID NO: 69);
   ii) the CDR1 sequence of GRTFTTY (SEQ ID NO: 18), the CDR2 sequence of RWSGGR (SEQ ID NO: 48) or WSGG (SEQ ID NO: 268), and the CDR3 sequence of DLAEYSGTYSSPADSPAGYDY (SEQ ID NO: 80) or LAEYSGTYSSPADSPAGYD (SEQ ID NO: 281);
   iii) the CDR1 sequence of GRTFTTYR (SEQ ID NO: 28), the CDR2 sequence of IRWSGGRT (SEQ ID NO: 58), and the CDR3 sequence of AADLAEYSGTYSSPADSPAGYDY (SEQ ID NO: 91);
   iv) the CDR1 sequence of GRTFTTYRMG (SEQ ID NO: 162), the CDR2 sequence of AIRWSGGRTLYADSVKG (SEQ ID NO: 192), and the CDR3 sequence of DLAEYSGTYSSPADSPAGYDY (SEQ ID NO: 223);
   v) the CDR1 sequence of TTYRMG (SEQ ID NO: 172), the CDR2 sequence of FVAAIRWSGGRTL (SEQ ID NO: 202), and the CDR3 sequence of AADLAEYSGTYSSPADSPAGYD (SEQ ID NO: 234); or
   vi) the CDR1 sequence of GRTFTTYRMG (SEQ ID NO: 182), the CDR2 sequence of AIRWSGGRTL (SEQ ID NO: 212), and the CDR3 sequence of DLAEYSGTYSSPADSPAGYDY (SEQ ID NO: 245); and
k) VHH12:
   i) the CDR1 sequence of FNTYAMG (SEQ ID NO: 9), the CDR2 sequence of SITWNGGSTSYADSVKG (SEQ ID NO: 39), and the CDR3 sequence of ARYYVSGTYFPANY (SEQ ID NO: 70);
   ii) the CDR1 sequence of GRTLSFNTY (SEQ ID NO: 19), the CDR2 sequence of TWNGGS (SEQ ID NO: 49) or WNGG (SEQ ID NO: 269), and the CDR3 sequence of ARYYVSGTYFPANY (SEQ ID NO: 81) or RYYVSGTYFPAN (SEQ ID NO: 282);
   iii) the CDR1 sequence of GRTLSFNTYA (SEQ ID NO: 29), the CDR2 sequence of ITWNGGST (SEQ ID NO: 59), and the CDR3 sequence of AAARYYVSGTYFPANY (SEQ ID NO: 92);
   iv) the CDR1 sequence of GRTLSFNTYAMG (SEQ ID NO: 163), the CDR2 sequence of SITWNGGSTSYADSVKG (SEQ ID NO: 193), and the CDR3 sequence of ARYYVSGTYFPANY (SEQ ID NO: 224);
   v) the CDR1 sequence of SFNTYAMG (SEQ ID NO: 173), the CDR2 sequence of FVASITWNGGSTS (SEQ ID NO: 203), and the CDR3 sequence of AAARYYVSGTYFPAN (SEQ ID NO: 235); or
   vi) the CDR1 sequence of GRTLSFNTYAMG (SEQ ID NO: 183), the CDR2 sequence of SITWNGGSTS (SEQ ID NO: 213), and the CDR3 sequence of ARYYVSGTYFPANY (SEQ ID NO: 246).

286. A method of modulating a function of pIgR in a cell comprising contacting the cell with the VHH domain of any of embodiments 252 to 265 or the therapeutic molecule of any of embodiments 266 to 274, including an effective amount of the VHH domain or therapeutic molecule.

287. The method of embodiment 286, wherein the modulating the function of pIgR in the cell is activating said function of pIgR in said cell.

288. The method of embodiment 286, wherein the modulating the function of pIgR in the cell is inhibiting said function of pIgR in said cell.

289. A method of delivering a molecule to a pIgR-expressing cell comprising contacting the cell with the VHH domain of any of embodiments 252 to 265 or the therapeutic molecule of any of embodiments 266 to 274, including an effective amount of the VHH domain or therapeutic molecule.

290. A method of delivering a molecule to a mucosal lumen of a subject, the method comprising administering to the subject the VHH domain of any of embodiments 1 to 14 or the therapeutic molecule of any of embodiments 266 to 274, including an effective amount of the VHH domain or the therapeutic molecule.

291. The method of any one of embodiments 286 to 290, wherein the cell is a mucosal epithelial cell.

292. The method of any one of embodiments 286 to 291, wherein the cell is a cancer cell.

293. The method of embodiment 292, wherein the cancer cell is a lung cancer cell, an esophageal cancer cell, a stomach cancer cell, a duodenal cancer cell, a liver cancer cell, a bladder cancer cell, a sinus cancer cell, a nasal cavity cancer cell, an endometrial cancer cell or a colorectal cancer cell.

294. The method of any one of embodiments 286 to 293, wherein the cell is in a subject.

295. A method of delivering a molecule to a mucosal lumen of a subject, the method comprising administering to the subject the VHH domain of any of embodiments 252 to 265 or the therapeutic molecule of any of embodiments 266 to 274, including an effective amount of the VHH domain or the therapeutic molecule.

296. The method of embodiment 295, wherein the mucosal lumen is in the lung or in the gastrointestinal tract of the subject.

297. A method of delivering a molecule to an organ of a subject, the method comprising administering to the subject a molecule comprising a VHH domain.

298. The method of embodiment 297, wherein the organ is selected from the group consisting of small intestine, large intestine, stomach, esophagus, salivary gland, lung, vagina, uterus, and lacrimal gland.

299. The method of embodiment 298, wherein the organ is a lung.

300. The method of any one of embodiments 289 to 299, wherein the molecule is an antibody or fragment thereof, a peptide, a vaccine, a small molecule, a polynucleotide, a molecule comprising a radioactive isotope, a toxin, an enzyme, an anticoagulant, a hormone, a cytokine, an anti-inflammatory molecule, an RNAi, an mRNA, a self-replicating RNA, an antibiotic, or an antibody-antibiotic conjugate.

301. The method of embodiment 300, wherein the molecule is an antibiotic, an antibody or fragment thereof, a peptide or a vaccine.

302. The method of embodiment 401, wherein the antibiotic is selected from the group consisting of a macrolide antibiotic, a fluoroquinolone, a tetracycline, amoxicillin, ceftriaxone, penicillin G, linezolid, moxifloxacin, and azithromycin.

303. The method of any one of embodiments 286 to 302, wherein the molecule is administered to the bloodstream of the subject.

304. The method of any one of embodiments 286 to 303, wherein the molecule is administered intravenously or subcutaneously.

305. A method to diagnose a disease or condition comprising administering the VHH domain of any of embodiments 252-265 or the therapeutic molecule of any of embodiments 266-274 to the subject, detecting the amount of VHH domain in a tissue of the subject, wherein the tissue comprises a diseased cell, and comparing the amount of VHH domain in the tissue of the subject with a reference amount of VHH domain in the tissue of a comparable healthy subject.

306. The method of embodiment 305, wherein the VHH domain of embodiments 1-14 comprises a radioisotope.

307. The method of embodiment 306, wherein the radioisotope is zirconium-89.

308. The method of any of embodiments 305 to 307, wherein the disease is lung cancer, and wherein the tissue is lung.

309. The method of any of embodiments 305 to 307, wherein the disease is endometrial cancer, and wherein the tissue is the uterus, or wherein the disease is an inflammatory disease, such as inflammatory bowel disease, Crohn's disease or ulcerative colitis, and wherein the tissue is Lamina propria.

310. The method of any of embodiments 305 to 307, wherein the disease is colon cancer, and wherein the tissue is the colon.

311. The method of any one of embodiments 308 to 310, wherein the diseased cell expresses an antigen, and wherein the therapeutic molecule is coupled to an antibody that specifically recognizes the antigen.

312. The method of embodiment 311, wherein the antigen is specific to the diseased cell.

313. A pharmaceutical composition comprising a means for increasing the rate of pIgR-mediated transcytosis (e.g., forward transcytosis or reverse transcytosis) across an epithelial cell, and a pharmaceutically acceptable carrier.

314. A pharmaceutical composition comprising a means for activating a function of pIgR in a cell, and a pharmaceutically acceptable carrier.

315. A pharmaceutical composition comprising a means for delivering a molecule to a mucosal lumen of a subject, or into systemic circulation in a subject, or into Lamina propria of a subject, and a pharmaceutically acceptable carrier.

316. The pharmaceutical composition of embodiment 315, wherein the molecule is an antibody or fragment thereof, a peptide, a vaccine, a small molecule, a polynucleotide, a molecule comprising a radioactive isotope, a toxin, an enzyme, an anticoagulant, a hormone, a cytokine, an anti-inflammatory molecule, an RNAi, an mRNA, a self-replicating RNA, an antibiotic, or an antibody-antibiotic conjugate.

### 6. EXAMPLES

The following examples are provided to further describe some of the embodiments disclosed herein. The examples are intended to illustrate, not to limit, the disclosed embodiments.

### 6.1 Example 1: Immunization, Recovery and Screening of pIgR Binders

To generate a panel of anti-pIgR single-domain antibodies, llamas were immunized with recombinant human pIgR (hpIgR) and/or mouse pIgR (mpIgR) for about 90 days. The whole blood and PBMCs was isolated from llamas, and RNA was prepared. After first-strand cDNA synthesis, llama-specific primers annealing to (i) the V_{H} (heavy-chain variable region), (ii) VHH leader sequence genes, and (iii) the CH2 gene were used to PCR amplify the V_{H} and VHH gene repertoires.

VHH repertoires were separated from V_{H} repertoires by running the PCR fragments on a gel and excising the smaller band. The VHH gene repertoire was reamplified and cloned into a CMV-based mammalian vector. The VHH-gene was formatted as Ig-fusion. The library was transformed in E. coli. Single colonies were picked in a 96-well format for Sanger sequencing. From approximately 300 unique sequences, a select number of VHH sequences were selected for miniprep DNA, and then scaled-up for future recombinant expression and screening. 39 clones were chosen for miniprep DNA from the mo_pIgR_llama_Sort1 campaign and 35 chosen from hu_pIgR_llama_Sort1 campaign. Clone Selection was based on sequence uniqueness (weighted heavily on CDR3) and a Framework 2 signature indicative of VHH or Heavy-Chain only derived sequence.

B-cells that were positive for VHH and antigen binding were isolated and recovered, cloned and the VHH variable domain were sequenced using established protocols. Following VHH-region sequencing, a panel of 73 VHH molecules were expressed and purified as fusions to the human IgG1 mono-Fc protein. The sequence of the human IgG1 mono-Fc protein is as follows:

(SEQ ID NO: 146)
SPAPELLGG PSVFLFPPK PKDTLMISRTP EVTCVVVDVS
HEDPEVKFNW YVDGVEVHNA KTKPREEQY NSTYRVVSVLT
VLHQDWLNGK EYKCKVSNKA LPAPIEKTIS KAKGQPREPQ
VYTKPPSREE MTKNQVSLSC LVKGFYPSDI AVEWESNGQP
ENNYKTTVP VLDSDGSFRLA SYLTVDKSRW QQGNVFSCSV
MHEALHNHYT QKSLSLSPGK

This VHH panel was screened for binding to hpIgR and mpIgR ectodomain by enzyme-linked immunosorbent assays (ELISAs) resulted in 40 positive hits.

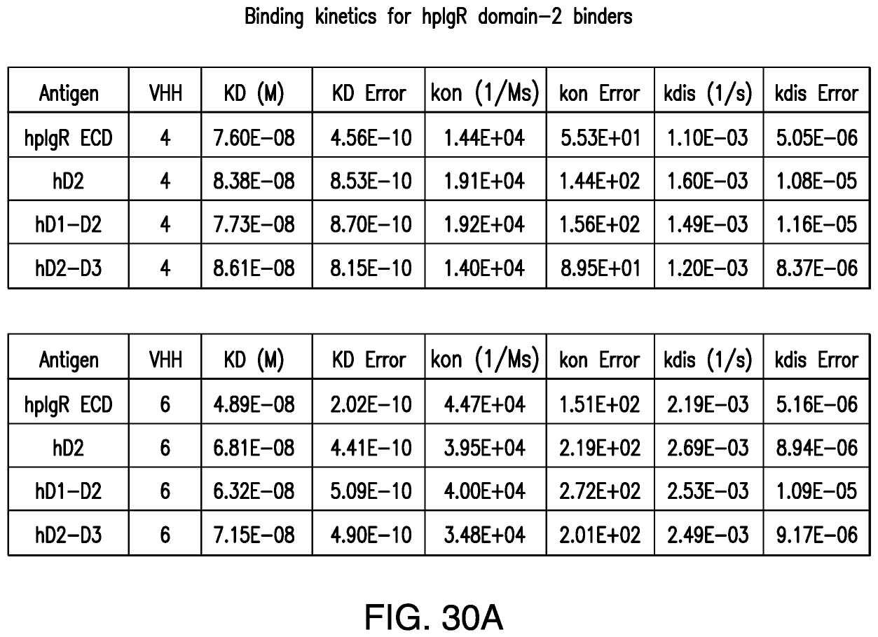

Bio-layer Interferometry was performed as follows. The ForteBioOctet RED384 system (Pall Corporation) was used to measure binding kinetics between VHH-mono-Fc molecules and pIgR proteins, and between IgA and pIgR proteins (in the absence and presence of VHH-mono-Fc molecules). Data were collected with Octet Data Acquisition version 7.1.0.87 (ForteBio) and analyzed using Octet Data Analysis version 7.1 (ForteBio). To measure binding kinetics between VHH-mono-Fc molecules and HIS-tagged pIgR proteins, VHH-mono-Fc was immobilized on amine-reactive generation-2 (ARG2) biosensors according to manufacturer's instructions and increasing concentrations of pIgR proteins were exposed to sensor-immobilized VHH. In some cases, HIS-tagged pIgR proteins were immobilized on anti-HIS biosensors and exposed to increasing concentrations of VHH-mono-Fc molecules. Association and dissociation rates were measured by the shift in wavelength (nm). For each sensor-immobilized protein, at least three different ligand concentrations were used, and K_{D} (equilibrium dissociation constant) was obtained by fitting the data to 1:1 binding model. All reactions were performed at 25° C. in PBS. The results are shown in FIGS. 30A-30B.

To measure binding kinetics between IgA and pIgR proteins, IgA was immobilized on ARG2 biosensors according to manufacturer's instructions, and immobilized IgA was exposed to increasing concentrations of pIgR ECD. To test the effect of VHH on pIgR-IgA binding, K_{D} values were measured for pIgR ECD binding to IgA in presence of VHH. IgA immobilized on ARG2 biosensors was exposed to increasing concentrations of pIgR-VHH complex, and association and dissociation rates were measured by the shift in wavelength (nm). For each sensor-immobilized IgA, at least three different pIgR or pIgR-VHH concentrations were used, and K_{D} (equilibrium dissociation constant) was obtained by fitting the data to 2:1 binding model. All reactions were performed at 25° C. in PBS.

Bio-layer interferometry showed that 14 binders from this panel had K_{D} values of <100 nM for binding to the mouse or human pIgR ectodomain (5 anti-mpIgR, 6 anti-hpIgR and 3 cross-reactive).

Expression and purification of VHH in CHO cells was performed as follows. DNA constructs for VHH were sub-cloned into mammalian expression vectors using the In-Fusion® HD Cloning Kit. ExpiCHO™ cells were transfected with the appropriate expression vectors. Supernatants were harvested after 6-7 days by centrifugation (4,000 g, 15 min), passed through a 0.45-um filter, and purified at 4° C. by MabSelect™ SuRe™ chromatography on an AKTA express system (both GE Healthcare) using DPBS (Sigma) as running buffer and 0.1 M sodium acetate, pH 3.5 as elution buffer. Elutions were immediately neutralized using 25% (v/v) 2 M Tris-HCl pH 7.0, dialyzed to DPBS, sterilized by 0.22-um filtration and stored at 4° C. Concentrations were determined by absorbance at 280 nm on a Nanodrop ND-1000 spectrophotometer (ThermoFisher Scientific). The results are shown in FIG. 14.

Cloning, expression and purification of pIgR constructs in HEK293 cells was performed as follows. Gene blocks-encoding desired hpIgR domain sequences were obtained from IDT and sub-cloned into mammalian expression vectors using the In-Fusion® HD Cloning Kit. HEK Expi293™ cells were transfected with pIgR-domain expression vectors using ExpiFectamine™ 293 transfection kit. Supernatants were harvested after 6-7 days by centrifugation (4,000 g, 15 min), passed through a 0.45-um filter and purified by immobilized metal ion chromatography using HisPur™ Cobalt resin (Thermo scientific). Buffer NPI-20 (Teknova) was used as running buffer and Buffer NPI-300 (Teknova) containing 300 mM Imidazole was used as elution buffer. Elutions were buffer exchanged to DPBS using PD10 desalting columns (GE health care) following manufacturer's instructions and purified pIgR domains were stored at 4° C. Concentrations were determined by absorbance at 280 nm on a Nanodrop ND-1000 spectrophotometer (ThermoFisher Scientific).

Analytical-SEC was performed as follows. All purified VHH-mono-Fc molecules were analyzed by analytical high-pressure liquid chromatography on an Agilent 1200 infinity system using an Agilent AdvanceBio Size exclusion column (300 Å, 2.7 um, 4.6×150 mm). Column was equilibrated with 0.2 M sodium phosphate pH 6.8 and 20 ul of samples were injected at a concentration of 0.5 mg/ml and at a flow rate of 0.35 mL/min. Monomeric VHH-mono-Fc elutes were detected at the expected retention time of ˜4 min at these settings. Data analysis was performed in OpenLab Chemstation to calculate % monomer content.

SEC-MALS was performed as follows. The molecular weight for purified VHH-mono-Fc molecules was measured by size-exclusion chromatography combined with multi-angle light scattering. The experiment was performed on a Waters high-pressure liquid chromatography instrument connected in series to Wyatt uDAWN light scattering/uTrEX instrument. An Acquity UPLC Protein BEH size-exclusion column (200 Å, 1.7 μm, 4.6×150 mm) was equilibrated with 1×DPBS pH 7.4 and 10 ul of samples were injected at a concentration of 0.5 mg/ml and at a flow rate of 0.3 mL/min. Molecular weight of the primary species (monomeric VHH-Fc) was calculated using the Astra software package (Wyatt).

### 6.2 Example 2: Biophysical Characterization of hpIgR-Specific Binders

10 pIgR binders (8 hpIgR specific and 2 human/mouse cross-reactive) from Example 1 were selected for further biophysical and functional assays. The 10 pIgR binders were expressed and purified from CHO cells using Protein-A affinity chromatography. Size-exclusion chromatography combined with multi-angle light scattering showed that molecular weight of 10 VHH-mono-Fc binders (VHH2, VHH3, VHH4, VHH5, VHH6, VHH7, VHH9, VHH10, VHH11, and VHH12) ranged from 41.3 kDa to 48.7 kDa.

Thermal stability of a sample was determined by differential scanning fluorimetry, specifically the NanoDSF method, using an automated Prometheus instrument. Measurements were made by loading a sample into a 24-well capillary from a 384-well sample plate. Duplicate runs were performed for each sample. A Prometheus NanoDSF user interface (Melting Scan tab) was used to set up the experimental parameters for the run. The thermal scans for a typical IgG sample spanned from 20° C. to 95° C. at a rate of 1.0° C./minute. Dual-UV technology monitoring of intrinsic tryptophan and tyrosine fluorescence at the emission wavelengths of 330 nm and 350 nm was undertaken. The F350 nm/F330 nm ratio was plotted against temperature to generate an unfolding curve.

The back reflection optics of the instrument was also used for the detection of sample aggregation. Such optics emitted near-UV light at a wavelength that is not absorbed by proteins. This light passed through the sample and was reflected to the detector. Protein aggregates scatter this light, and thus only non-scattered light reaches the detector. The reduction in back reflected light was a direct measure for aggregation in the sample and is plotted as mAU (Attenuation Units) against temperature. Nano DSF was used for measuring thermal unfolding parameters (Tm and Tagg) of VHH binders at 0.5 mg/mL concentration in Phosphate Buffered Saline, pH 7.4.

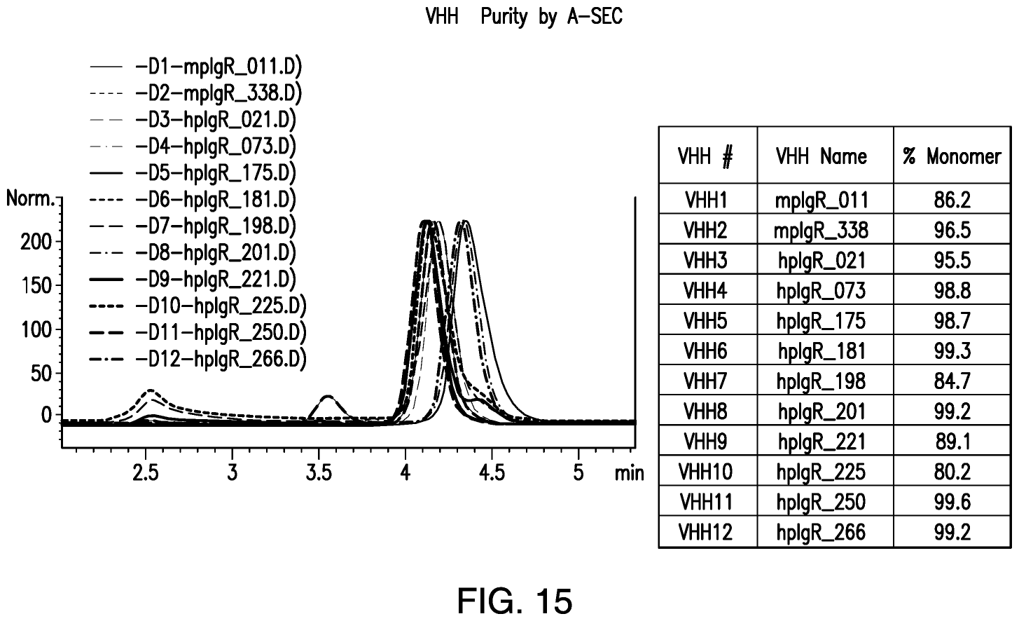

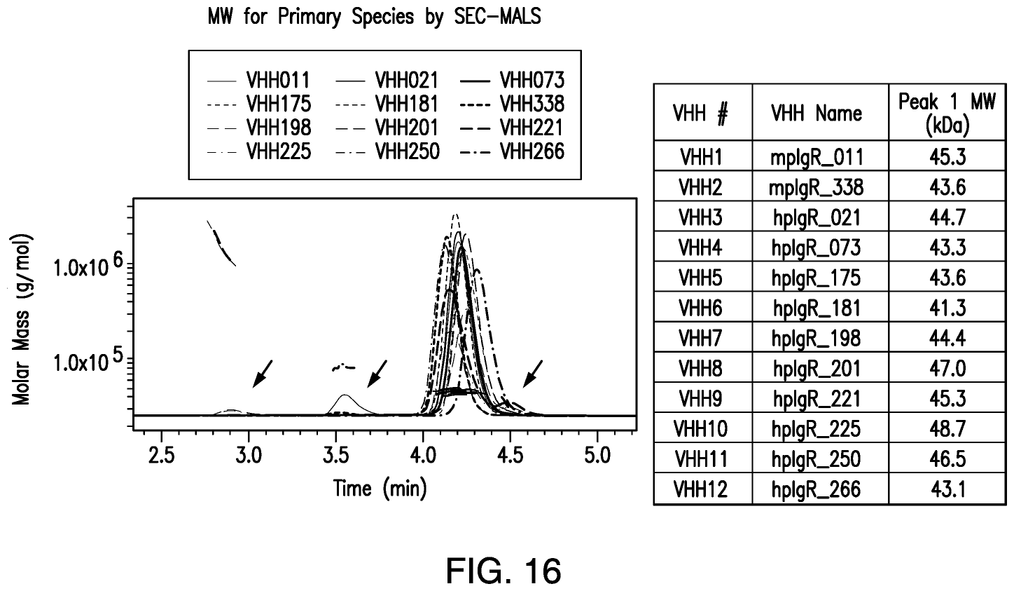

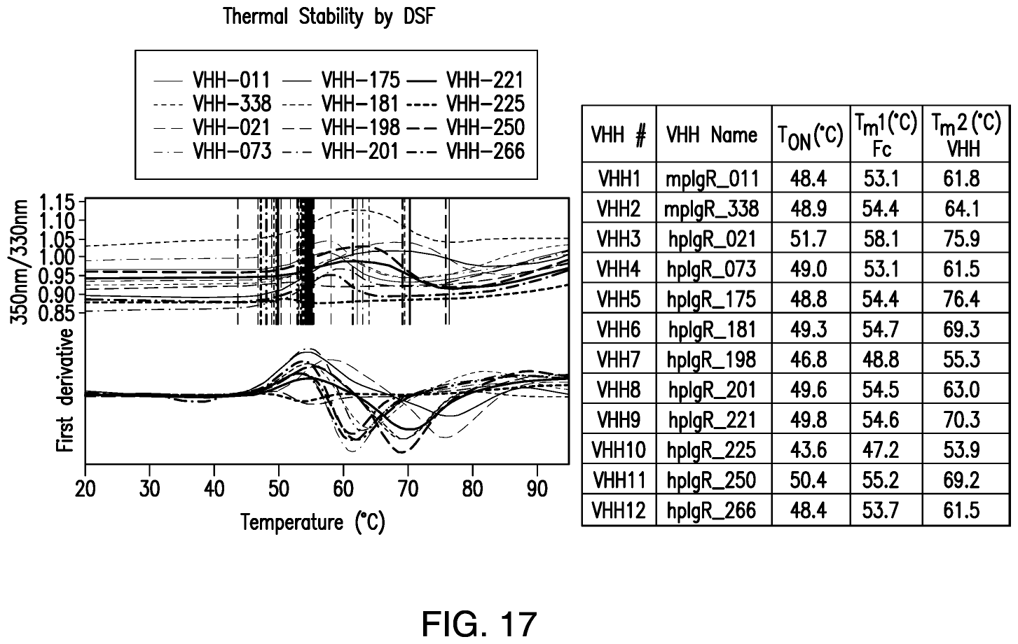

VHH-mono-Fc molecules were expressed in CHO cells and purified using Protein-A affinity chromatography. Homogeneity and molecular weight of the purified proteins were verified by analytical size-exclusion chromatography (A-SEC) and size-exclusion chromatography combined with multiple-angle light scattering (SEC-MALS), respectively. The results for A-SEC are shown in FIG. 15. The results for SEC-MALS are shown in FIG. 16. Thermal stability was assessed by differential scanning fluorimetry (DSF), with results shown in FIG. 17. The Tₘ for VHH molecules is reported below. K_{D} values for VHH-hpIgR ectodomain interactions were measured by bio-layer interferometry. EC₅₀ values for VHH molecules binding to MDCK-hpIgR cells were measured by flow cytometry.

Flow Cytometry was performed as follows. To test whether VHH-mono-Fc molecules recognize cell-surface hpIgR, Madin-Darby canine kidney (MDCK) cells engineered to express full-length hpIgR were used. Cells were cultured in Dulbecco's modified Eagle's medium containing 10% fetal calf serum at 37° C. with 5% CO₂. Cells were split into equal fractions (≈70,000 cells) and incubated with increasing concentrations of VHH-mono-Fc molecules for 30 min at 4° C. Cells were washed twice with cold PBS (pH 7.4) and incubated with a fluorescently-labelled anti-Fc antibody (Alexa Fluor® 647 AffiniPure F(ab′)₂ fragment Goat Anti-Human IgG Fcγ Fragment Specific) for 30 min in staining buffer (2 μg/ml Ab) at 4° C. Cells were washed twice with cold staining buffer, resuspended in running buffer and analyzed with an iQue Screener (IntelliCyt Corporation). Binding was assessed by RL1 (A647) Geomeans from the live cell population and EC50 was calculated by fitting log VHH concentration versus MFI in Prism (Graphpad).

The data are shown in Table 1 below.

TABLE 1

| | Tₘ | K_{D} | EC₅₀ | mpIgR |
|---|---|---|---|---|
| VHH | (° C.) | (nM) | (nM) | binding? |
| VHH2 | 64.1 | 21 | 6.3 | Yes |
| VHH3 | 75.9 | 5 | 6.4 | Yes |
| VHH4 | 61.5 | 22 | 32.9 | No |
| VHH5 | 76.4 | 11 | 4.3 | No |
| VHH6 | 69.3 | 27 | 11.5 | No |
| VHH7 | 55.3 | 521 | 36.4 | No |
| VHH9 | 70.3 | 4 | 1.5 | No |
| VHH10 | 53.9 | 256 | 20.4 | No |
| VHH11 | 69.2 | 19 | 1.5 | No |
| VHH12 | 61.5 | 34 | 4.6 | No |

In Table 1, differential scanning fluorimetry showed that Tₘ values of 10 VHH molecules ranged from 53.9° C. to 76.4° C. Differential scanning fluorimetry showed that Tₘ values of five potent VHH binders ranged from 61° C. to 70° C. Bio-layer interferometry showed that 8 binders from this panel had K_{D} values of <50 nM for binding to the human pIgR ectodomain, as shown in Table 1. Also, flow cytometry showed that 6 binders had EC₅₀ values of <10 nM for binding to MDCK-hpIgR cells.

### 6.3 Example 3: Cell Binding and Transcytosis Assay

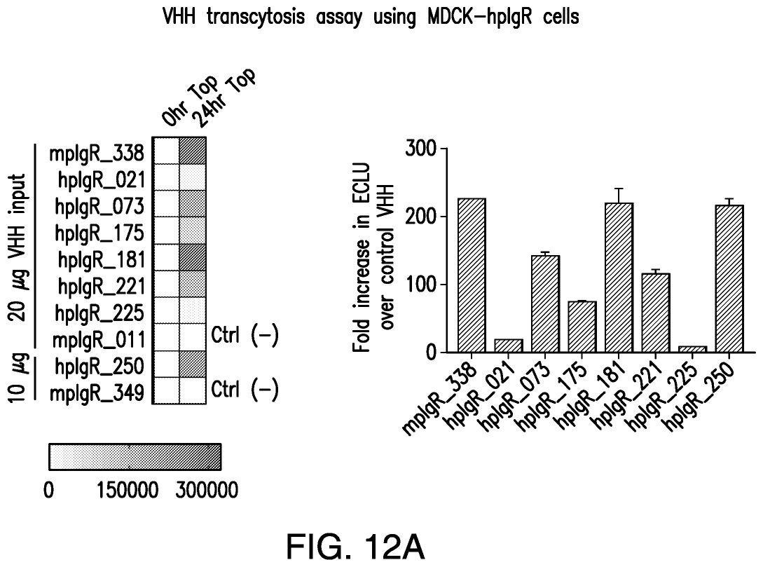

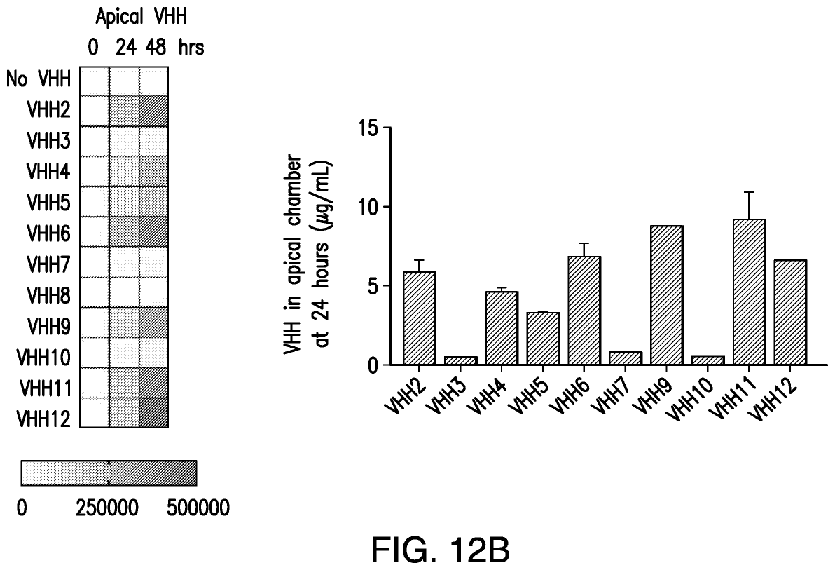

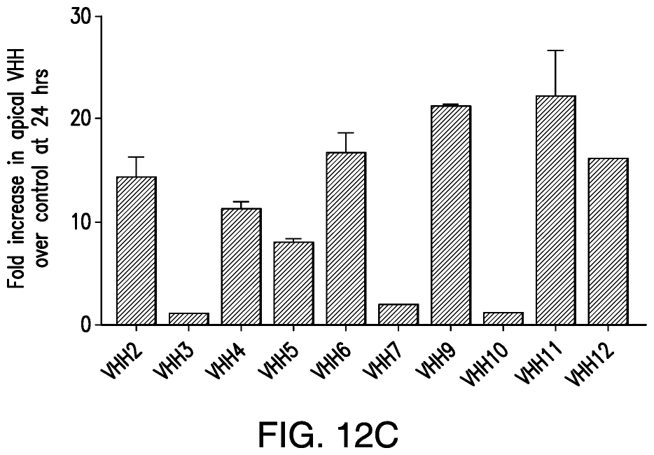

A transcytosis assay was performed as follows. Madin-Darby canine kidney (MDCK) cells, a commonly used epithelia model system, were used to investigate if VHH binders could be transported across epithelia by pIgR mediated transcytosis. MDCK cells, un-transfected or stably transfected with human pIgR were used to study transcytosis (See Natvig, I. B., Johansen, F. E., Nordeng, T. W., Haraldsen, G. & Brandtzaeg, P. Mechanism for enhanced external transfer of dimeric IgA over pentameric IgM: studies of diffusion, binding to the human polymeric Ig receptor, and epithelial transcytosis. J. Immunol. 159, 4330-4340 (1997)). Expression of hpIgR in MDCK cells and monolayer formation were confirmed by confocal laser microscopy. Approximately 5.0×10⁵ cells were seeded on 1-cm², 3.0-μm collagen-coated PTFE filters (Transwell-COL 3494; Costar). The cells were incubated for 3 days at 37° C. with 5% CO₂ in Dulbecco's modified Eagle's medium containing 10% fetal calf serum, 50 μg/ml gentamicin, and 1 mM L-glutamine. 20 μg of test VHH-mono-Fc molecules were added to the basolateral chamber, and the filters were incubated for 24 or 48 hours at 37° C. in fresh medium. A VHH-mono-Fc that did not bind to pIgR (irrelevant VHH) was used as a control together with 100 nM (15 μg/mL) human IgG (to control for unspecific transport and leakage). The apical medium was harvested, and the amount of VHHmono-Fc, transported by pIgR, was calculated by standard titration studies. IgG leakage to the apical medium was detected by MSD. The results of the transcytosis assay are shown in FIGS. 12A-12B.

Additionally, a biotinylated anti-VHH antibody was used to capture VHH-mono-Fc on streptavidin plates and a ruthenylated anti-Fc antibody to detect VHH-mono-Fc by the MSD platform. The results of this assay are shown in FIG. 12C. Six VHHs (2, 4, 6, 9, 11 and 12) showed >10-fold increase in their apical concentration relative to control VHH.

### 6.4 Example 4: Transcytosis Assays Using Primary Human Lung Tissue Model

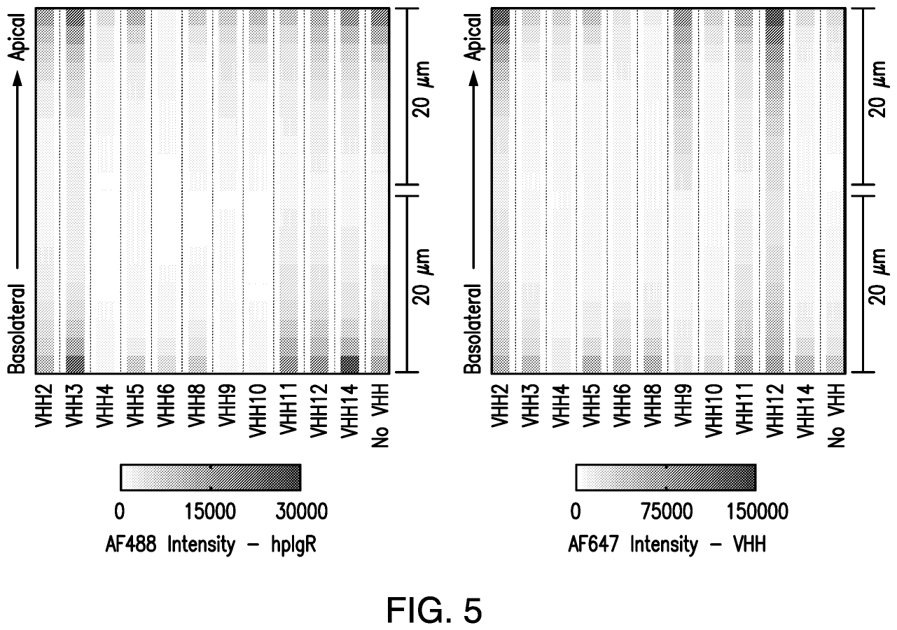

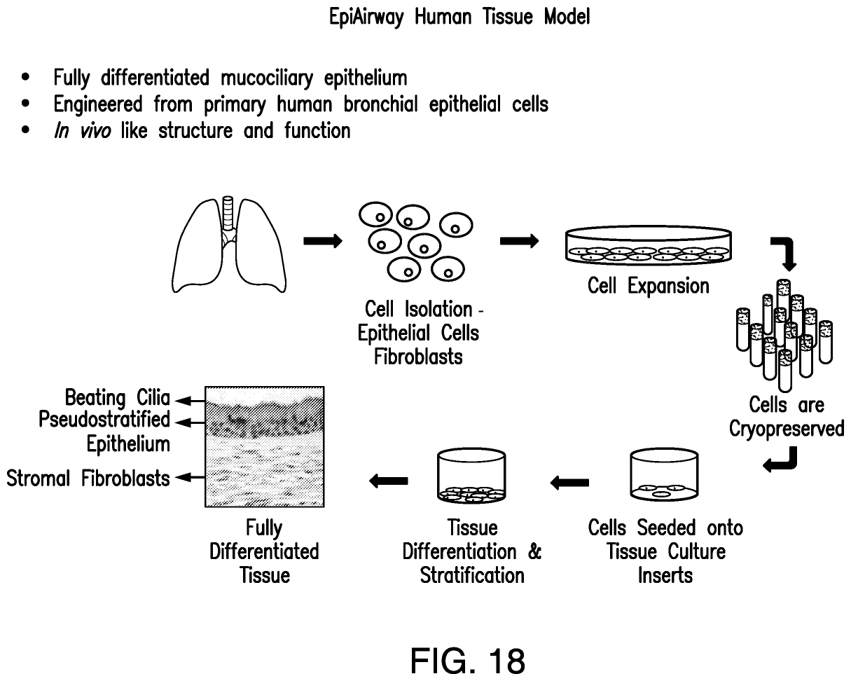

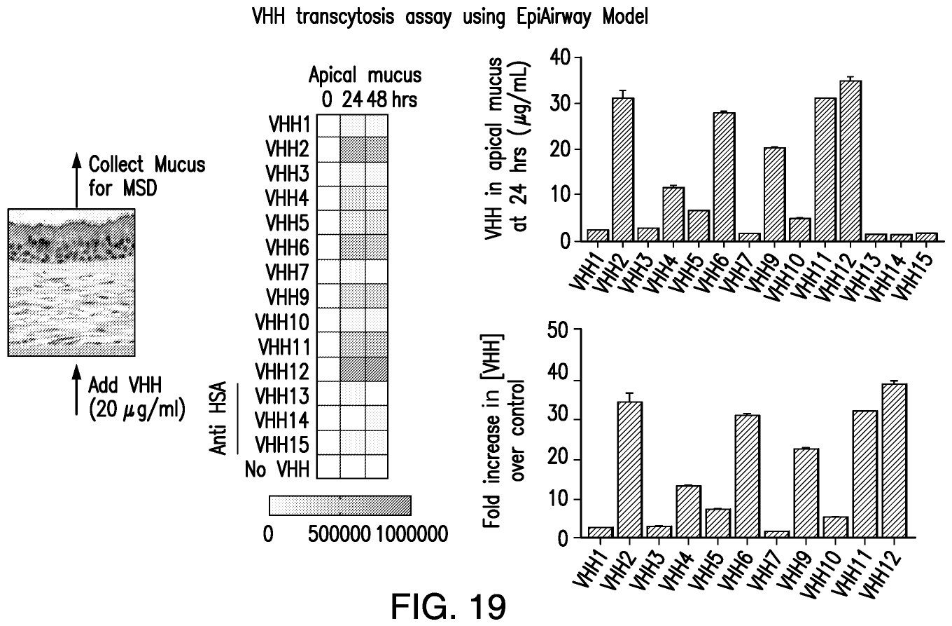

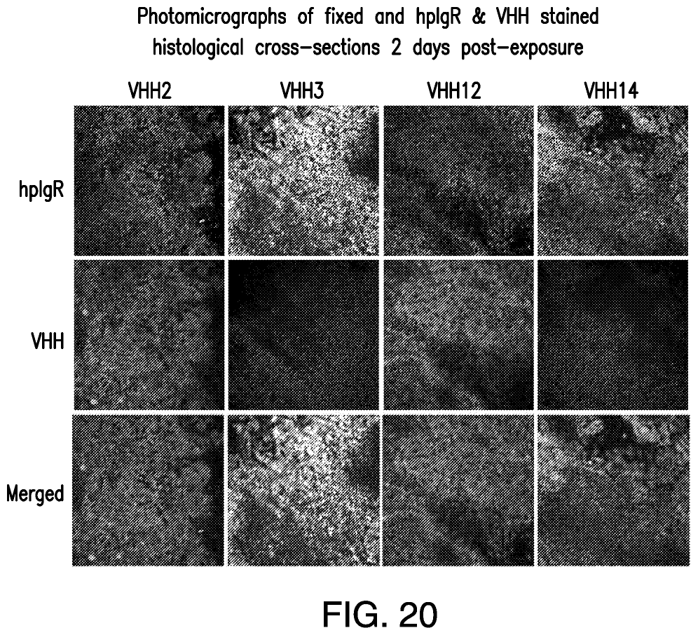

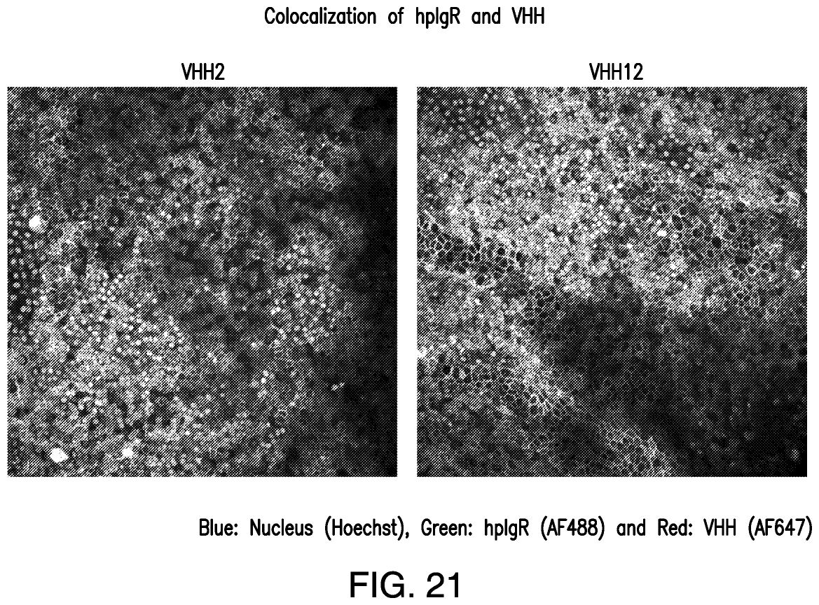

The EpiAirway human lung tissue model was also used to test the transcytosis activity of 10 VHH molecules from the basolateral to the apical epithelium and their delivery to the mucosal lumen. The EpiAirway model is depicted in FIG. 18. The EpiAirway model is an established lung tissue model engineered from primary human tracheal bronchial cells. Tissue models were obtained from Mattek Corporation and maintained according to manufacturer's instructions. 20 μg of test and control VHH-mono-Fc molecules were added to 1 ml of EpiAirway media in the basolateral chamber and 100 ul of samples were collected from the basolateral and apical chambers at 0, 24 and 48 hours. EpiAirway TEER buffer was used to collect the mucus from the apical chambers. The amount of VHH-mono-Fc present in basolateral media and apical mucus was quantified by electrochemiluminescence method. In this method, streptavidin MSD plates were coated with a biotinylated anti-VHH antibody (2 μg/ml in PBS) for 1 hour at RT with 1000 rpm, washed 3× with PBT, incubated with blocking buffer for 1 hour at RT, incubated with VHH-mono-Fc containing media/mucus (at different dilutions) for 2 hours at RT with 1000 rpm, washed 3× with PBT, incubated with ruthenylated-anti-human-Fc antibody (2 μg/ml in PBS) for 1 hour at RT with 1000 rpm, washed 3× with PBT and read plates in 40 ul reading buffer using the MSD imager. The amount of VHH-mono-Fc in basolateral and apical chambers was calculated by plotting ECLU values against VHH-mono-Fc standard curves in Prism (Graphpad). The data is shown in FIG. 19. A similar experiment in which IgG and IgA were transcytosed is shown in FIG. 20. Each photomicrograph in FIG. 20 is a representative image of one of the squares in the heat map in FIG. 5.

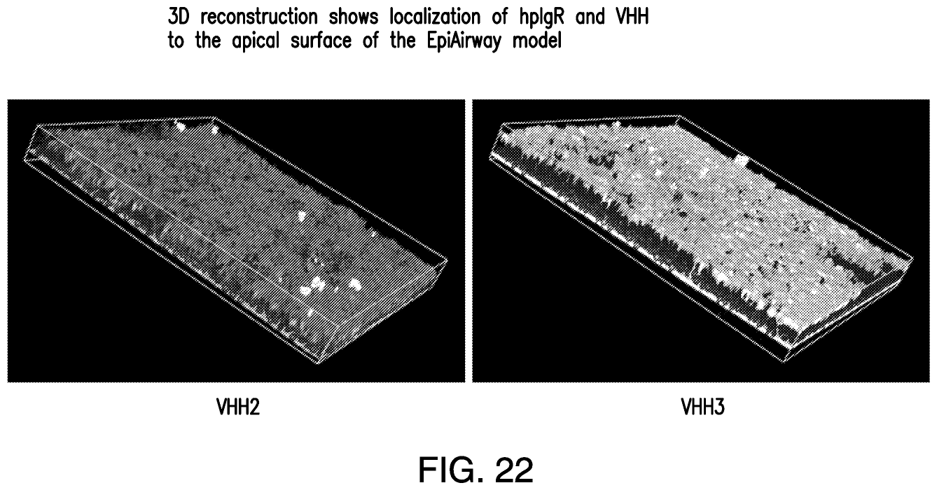

FIG. 22 shows 3D reconstruction shows localization of hpIgR and VHH to the apical surface of the EpiAirway model.

The amount of VHH present in the apical mucus 0, 24 and 48 hours post treatment was quantified by the electrochemiluminescence.

The Electrochemiluminescence assay was performed as follows. A meso-scale discovery (MSD) platform was used for conducting epitope mapping and epitope burial studies. To test the binding of VHH-mono-Fc molecules to purified pIgR protein constructs, Streptavidin MSD plates were coated with a biotinylated anti-HIS antibody (2 μg/ml in PBS) for 1 hour at RT with 1000 rpm, washed 3× with PBT (PBS+0.1% Tween-20), incubated with blocking buffer for 1 hour at RT, incubated with His-tagged pIgR proteins (10 μg/ml in PBS) for 2 hours at RT with 1000 rpm, washed 3× with PBT, incubated with VHH-mono-Fc molecules (100 μg/ml in PBS) for 2 hours at RT with 1000 rpm, washed 3× with PBT, incubated with ruthenylated-anti-human-Fc antibody (2 μg/ml in PBS) for 1 hour at RT with 1000 rpm, washed 3× with PBT and read plates in 40 ul reading buffer using the MSD imager. ECLU values were plotted as a heatmap.

To check whether VHH recognizes a buried epitope on pIgR, EC₅₀ values were measured for VHH-mono-Fc molecules binding to hpIgR-ECD protein by electrochemiluminescence using two different detection antibodies, an anti-Fc antibody and an anti-VHH antibody. pIgR ECD (10 μg/ml in PBS) was coated on high-bind MSD plates for 2 hours at RT with 1000 rpm, incubated with blocking buffer for 1 hour at RT, incubated with VHH-mono-Fc molecules (increasing concentrations in PBS) for 2 hours at RT with 1000 rpm, washed 3× with PBT, incubated with ruthenylated secondary antibody (2 μg/ml in PBS) for 1 hour at RT with 1000 rpm, washed 3× with PBT and read plates in 40 ul reading buffer using the MSD imager. EC50 was calculated by fitting log VHH concentration versus log ECLU in Prism (Graphpad). The increase in EC₅₀ (>50-fold) due to anti-VHH detection was used as a measure to determine whether VHH recognized buried epitope on pIgR.

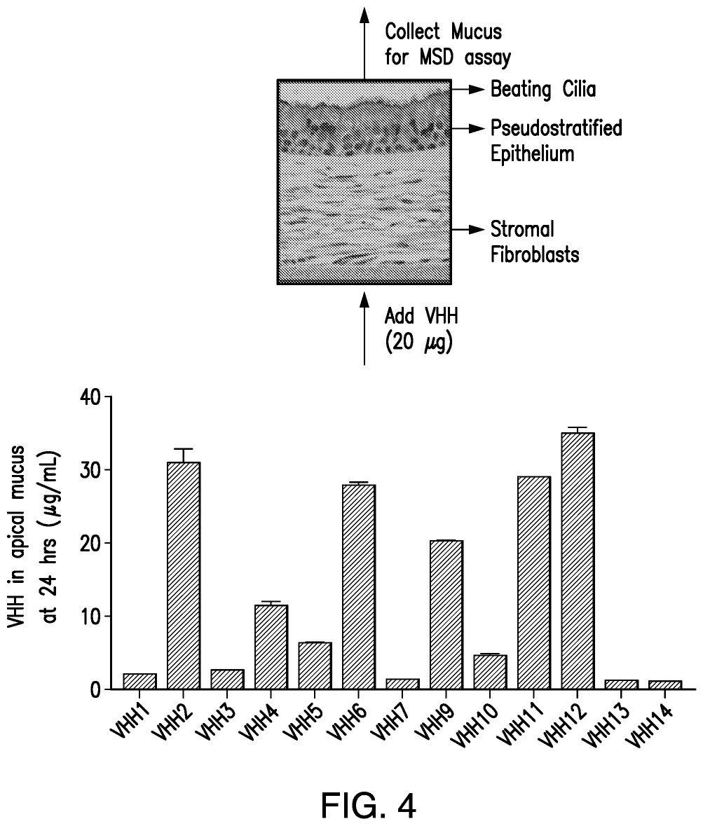

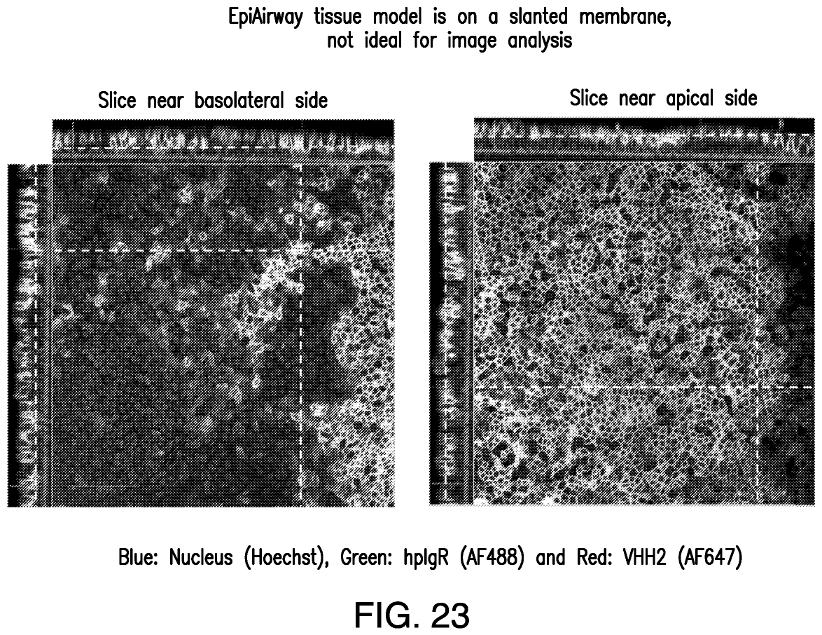

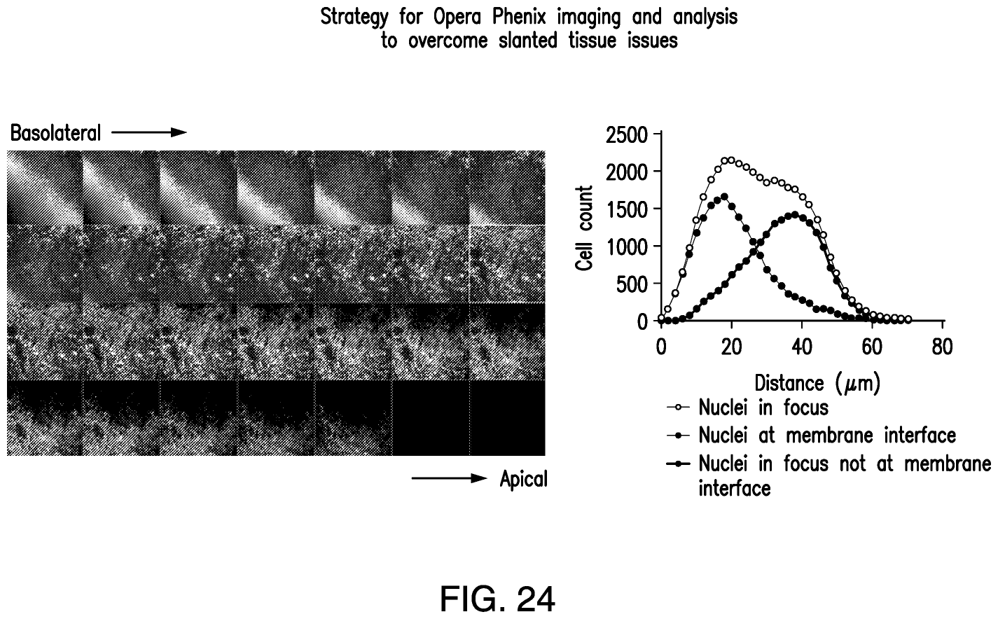

At 48 hours post-treatment, tissue samples were fixed, permeabilized and stained for tracking hpIgR and VHH across the EpiAirway model. The data is shown in FIG. 4. Five VHH molecules (VHH2, VHH6, VHH9, VHH11 and VHH12) showed greater than 20-fold increase in their mucosal amount relative to control VHH molecules. For the best pIgR agonist (VHH12), 17.5% of basolateral VHH input was secreted into mucus every 24 hours. FIG. 23 shows that the EpiAirway tissue model is on a slanted membrane, which is not ideal for image analysis. FIG. 24 illustrates a strategy for Opera Phenix imaging and analysis to overcome slanted tissue issues with EpiAirway tissue model.

Following transcytosis, indirect immunofluorescence was used to trace the location and amount of hpIgR and VHH across the EpiAirway tissue model by Opera Phenix confocal laser microscopy. Indirect immunofluorescence was used to track the amount of pIgR and VHH-mono-Fc retained across the EpiAirway model two-days post-treatment. Tissue samples were rinsed in PBS, tissues were fixed with 2 ml of 10% Formalin at RT for 20 minutes, washed three times with 2 ml PBST (1% Triton-X100 in PBS) at RT for 10 minutes each (with gentle agitation), incubated with primary antibodies (500 ul apical, 500 ul basolateral) diluted in PBTG (PBST with 10% goat serum) for 2 hours at RT (with gentle agitation), washed two times with 2 ml PBTG at RT for 10 minutes each (with gentle agitation), incubated with secondary antibodies (100 ul apical, 100 ul basolateral) diluted in PBTG for 1 hour at RT (with gentle agitation) and washed two times with 2 ml PBTG at RT for 10 minutes each (with gentle agitation). The primary antibody mix contained mouse anti-pIgR antibody and biotinylated anti IgA antibody both at 5 μg/ml. The secondary antibody mix contained Alexa-Flour 488-labelled anti-mouse antibody (1:100 dilution), Alexa-Flour 647-labelled streptavidin (1:100 dilution) and Hoechst (1:1000 dilution). Fixed, permeabilized and stained tissues were imaged at 20× resolution (30-40 planes, 2 um distance) using Opera Phenix confocal laser microscopy. Image analysis was performed using the Harmony suite, fluorescence readouts were corrected for membrane auto-fluorescence, normalized for number of cells and plotted as heat maps in Prism (Graphpad).

The data is shown in FIG. 5. Imaging studies corroborated transcytosis results and showed colocalization of hpIgR and VHH, especially closer to the apical epithelium. Since pIgR is proteolytically cleaved and released into mucus upon transcytosis, the amount of tissue-retained pIgR inversely correlated with VHH function.

In the EpiAirway model, the presence of IgA did not affect the transcytosis of VHH9, however the presence of IgA had a negative effect on the four other VHH binders VHH2, VHH6, VHH11 and VHH12.

### 6.5 Example 5: Domain-Level Epitope Mapping

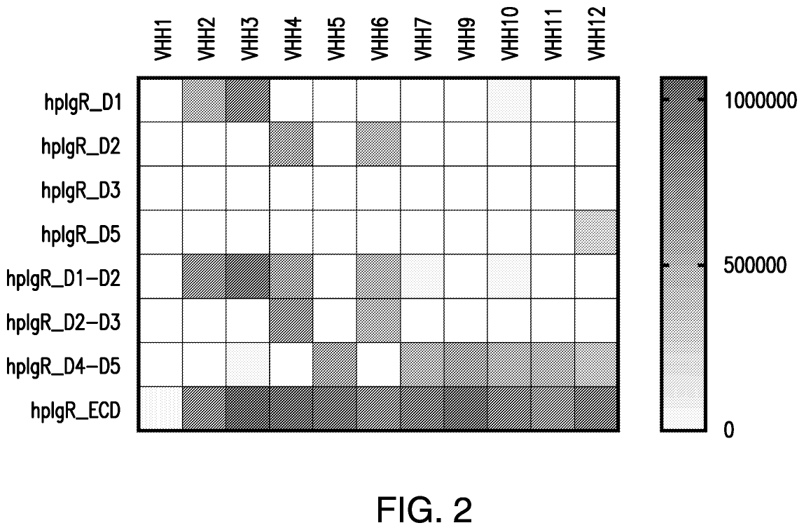

To conduct domain-level epitope mapping of VHHs, seven HIS-tagged hpIgR constructs (D1, D2, D3, D5, D1-D2, D2-D3 and D4-D5) were expressed and purified each encoding one or two domains of hpIgR ECD from HEK293 cells using immobilized metal ion affinity chromatography. Two constructs, D4 and D3-D4, showed poor expression and purification and were not used for epitope mapping assays. Binding of VHH-mFc molecules were tested to immobilized pIgR constructs by the electrochemiluminescence method. Results from the binding assay are shown as a heat map in FIG. 2.

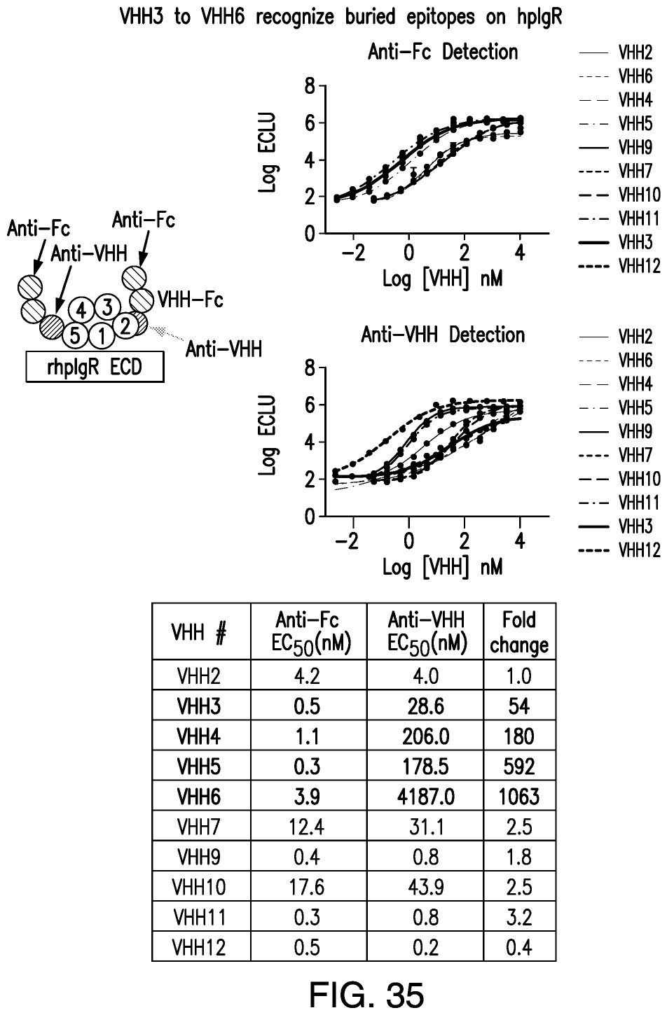

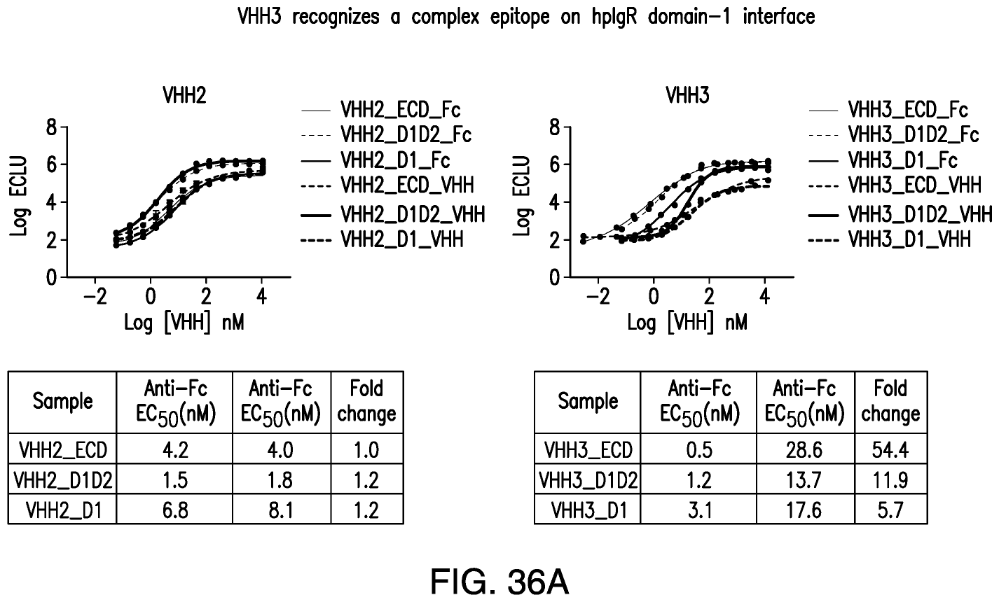

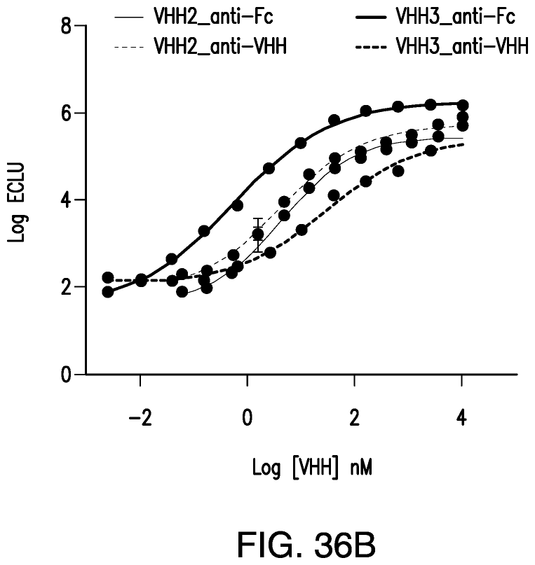

Recognition of buried epitopes by pIgR binders was performed as follows. The EC₅₀ for VHH-mono-Fc molecules binding to hpIgR-ECD protein was measured by electrochemiluminescence using two different detection antibodies, an anti-Fc antibody and an anti-VHH antibody. The increase in EC₅₀ (>50-fold) due to anti-VHH detection was used as a measure to determine whether VHH recognized buried epitope on pIgR. Four molecules (VHH3, VHH4, VHH5 and VHH6) recognized buried epitopes on pIgR, as shown in FIG. 35. As shown in FIGS. 36A-36B, VHH3 recognizes a complex epitope on the hpIgR domain-1 interface, and in particular, while no differences in EC₅₀ were observed for VHH2 (4 nM for both detection antibodies), VHH3 showed a 54-fold increase in EC₅₀ due to anti-VHH detection. Together these experiments indicated that VHH2 and VHH3 recognize domain-1 in a different fashion, that could have attributed to their differences in function.

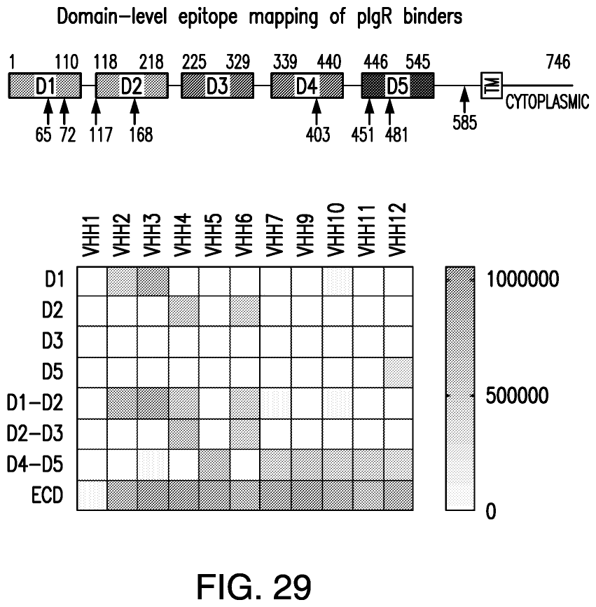

Epitope mapping showed that VHH2, VHH6 and VHH12 binds hpIgR domain 1, 2 and 5, respectively, whereas VHH9 and VHH11 binds to hpIgR domains 4-5. To test whether the VHH binding region recognizes buried epitopes on hpIgR, an electrochemiluminescence method using two different detection antibodies, an anti-Fc antibody and an anti-VHH antibody were used to generate EC₅₀ values that reflect VHH-mono-Fc molecules binding to hpIgR-ECD protein. An increase in EC₅₀ (>50-fold) due to anti-VHH detection was used as a measure to determine whether VHH recognized buried epitope on pIgR. The results are shown in Table 2 and FIG. 29.

TABLE 2

| | Anti-Fc | Anti-VHH | Fold change | Buried |
|---|---|---|---|---|
| VHH# | EC₅₀ (nM) | EC₅₀ (nM) | in EC₅₀ | epitope? |
| VHH2 | 4.2 | 4.0 | 1.0 | No |
| VHH3 | 0.5 | 28.6 | 54 | Yes |
| VHH4 | 1.1 | 206.0 | 180 | Yes |
| VHH5 | 0.3 | 178.5 | 592 | Yes |
| VHH6 | 3.9 | 4187.0 | 1063 | Yes |
| VHH7 | 12.4 | 31.1 | 2.5 | No |
| VHH9 | 0.4 | 0.8 | 1.8 | No |
| VHH10 | 17.6 | 43.9 | 2.5 | No |
| VHH11 | 0.3 | 0.8 | 3.2 | No |
| VHH12 | 0.5 | 0.2 | 0.4 | No |

The results of Table 2 indicate that four molecules (VHH3, VHH4, VHH5 and VHH6) recognized buried epitopes on pIgR. To conduct domain-level epitope mapping, seven HIS-tagged pIgR ectodomain constructs (D1, D2, D3, D5, D1-D2, D2-D3 and D4-D5) were successfully expressed and purified from HEK293 cells using immobilized metal ion affinity chromatography. The sequences of D1, D2, D3, D5, D1-D2, D2-D3, and D4-D5 comprise those of SEQ ID NOS: 216-222.

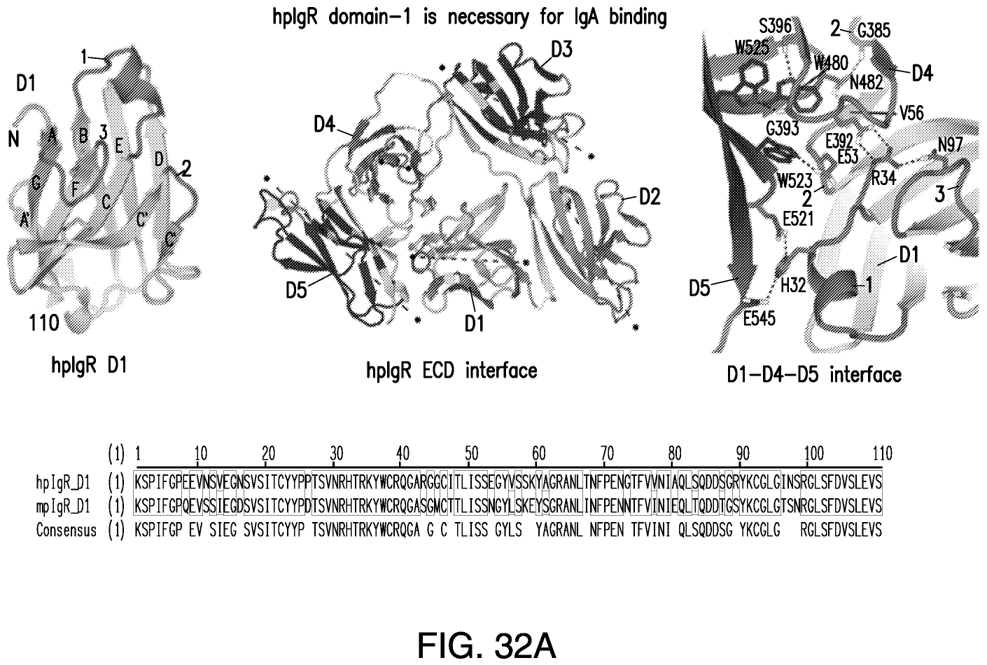

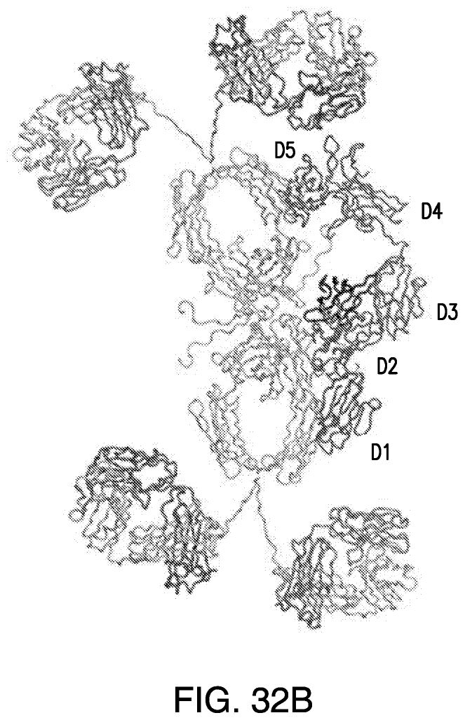

The binding of VHH-mono-Fc molecules to immobilized pIgR constructs is summarized as a heat map in FIG. 2. In brief, the epitopes of VHH2 and VHH3 are primarily contained within hpIgR domain 1 (D1), and the epitopes of VHH4 and VHH6 are primarily contained within hpIgR domain 2 (D2). As shown in FIG. 32A, D1 is necessary for IgA binding to hpIgR. The epitopes of other six VHH molecules are primarily contained within hpIgR domains 4-5 (D4-D5). Additionally, solution x-ray scattering studies conducted by Bonner et al., Mucosal Immunol., 2:74-84 (2009) suggest that upon interaction with dIgA, pIgR takes on an extended conformation, with domain-1 interacting with the Cα2 domain of one Fcα subunit and domain-5 binding the Cα2 subunit on the same side of the opposite Fcα subunit (FIG. 32B).

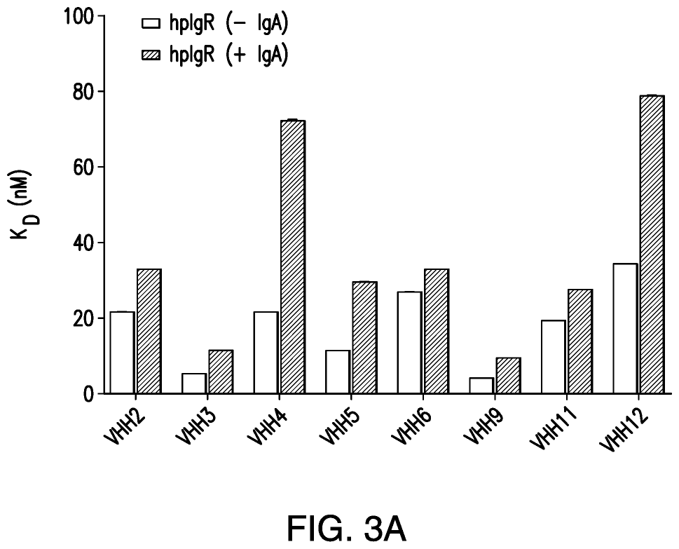

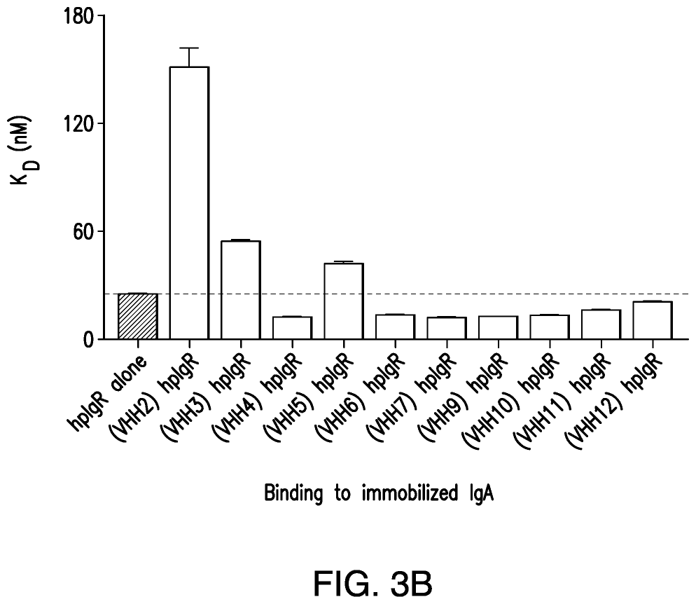

Next, competition binding assays were conducted for eight VHH-mono-Fc molecules that displayed K_{D} values of <100 nM for binding to hpIgR. First, to test the influence of IgA on hpIgR-VHH binding, K_{D} values were measured for full-length hpIgR ECD binding to immobilized VHH-mono-Fc molecules in the absence and presence of dIgA2 by bio-layer interferometry (FIG. 3A). In total, VHHs showed a 1.3 to 3.3-fold decrease in affinity for binding to hpIgR ECD due to the presence of dIgA. Pre-bound IgA had a small negative effect on binding of VHHs to pIgR, possibly due to steric hindrance arising from bound dIgA or conformational rearrangement of hpIgR ECD. Second, to test the effect of VHH on dIgA2 binding to hpIgR, K_{D} values for a recombinant dimeric IgA2 construct binding to the hpIgR ectodomain were measured with and without the presence of VHH-mono-Fc molecules. Three molecules (VHH2, VHH3 and VHH5) had a negative effect on IgA binding to pIgR, while other VHH molecules displayed a small positive effect on IgA binding to pIgR, as shown in FIG. 3B.

### 6.6 Example 6: VHH/IgA Competition Studies (Binding and Transcytosis)

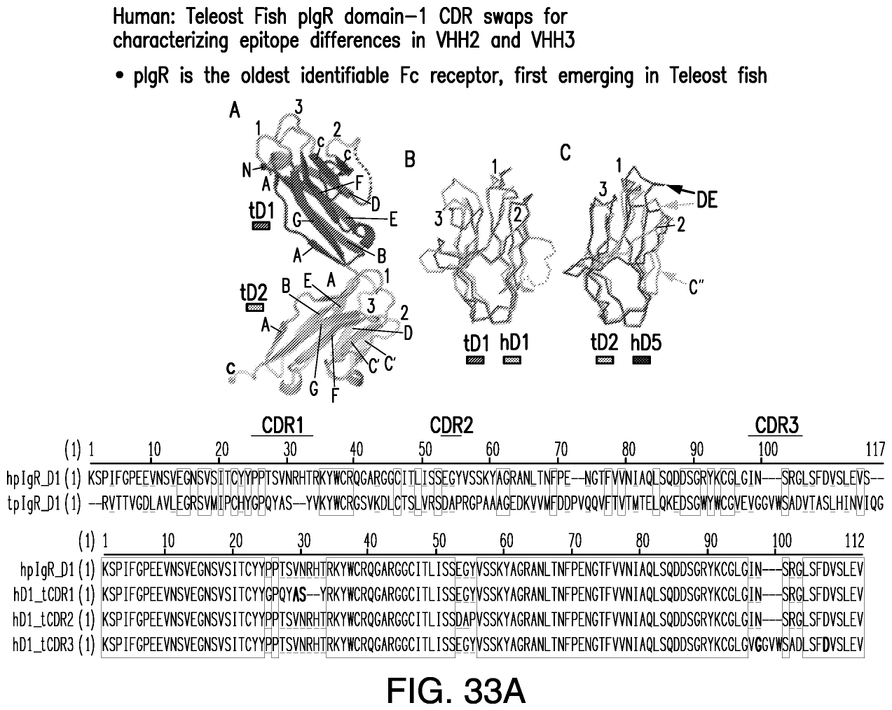

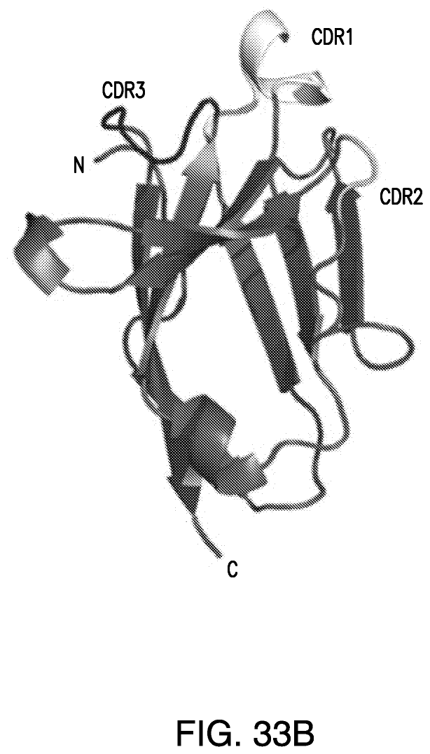

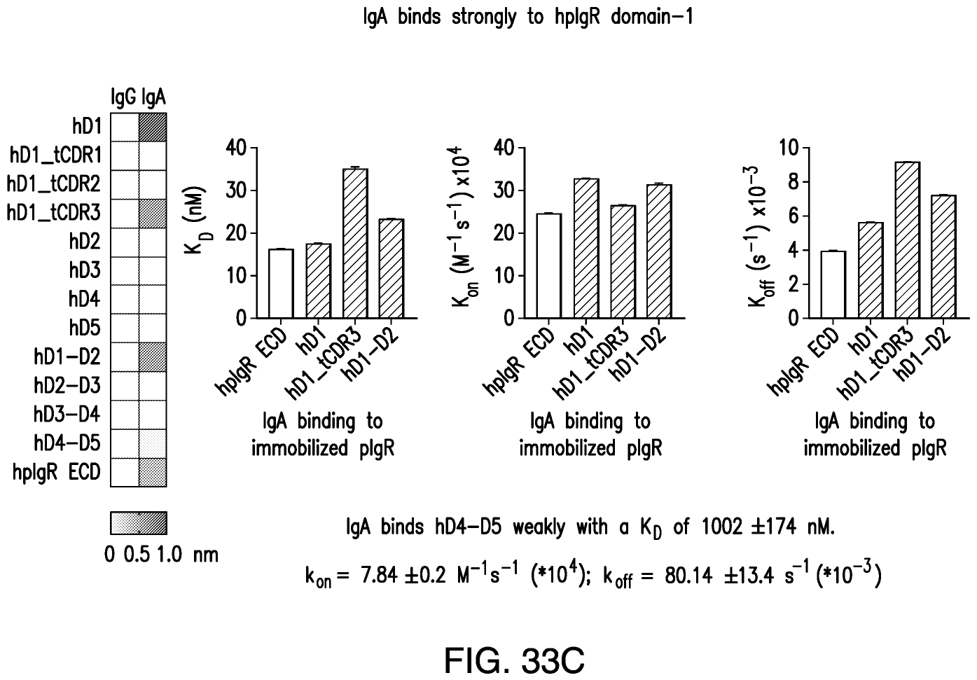

The differences in binding between VHH2, the transcytosis-positive domain-1 binder described above, and VHH3, a transcytosis-negative domain-1 binder, were compared. VHH3 binds stronger than VHH2. To test the importance of hpIgR domain-1 CDRs on VHH2 and VHH3 binding, each domain-1 CDR of human pIgR was swapped with the respective domain-1 CDR of teleost fish pIgR to make three new CDR-swapped hpIgR domain-1 constructs for use in binding studies. (Full-length hpIgR ECD was purchased from R&D Systems.) The five constructs (D1-D2, D1, D1_tCDR1, D1_tCDR2, D1_tCDR3) were expressed and purified from HEK293 cells using immobilized metal ion affinity chromatography. Three hpIgR domain-1 CDR mutants (D1_tCDR1, D1_tCDR2, D1_tCDR3) contain respective teleost fish CDR on a hpIgR domain-1 framework. His-tagged pIgR constructs were immobilized on anti-HIS biosensors and binding of VHH-mono-Fc molecules to pIgR constructs were measured by bio-layer interferometry. The data is shown in Table 3 and FIGS. 33A-33D.

TABLE 3

| | | Fold | | Fold |
|---|---|---|---|---|
| | VHH2 | change in | VHH3 | change |
| pIgR | K_{D} (nM) | K_{D} | K_{D} (nM) | in K_{D} |
| hpIgR_ECD | 22 | 1 | 10 | 1 |
| hpIgR_D1-D2 | 23 | 1 | 13.3 | 1.3 |
| hpIgR_D1 | 15.5 | 0.7 | 7.7 | 0.8 |
| hpIgR_D1_tCDR1 | 21.3 | 1 | 77 | 7.7 |
| hpIgR_D1_tCDR2 | No binding | No binding | No binding | No binding |
| hpIgR_D1_tCDR3 | 14.9 | 0.7 | 7.5 | 0.8 |

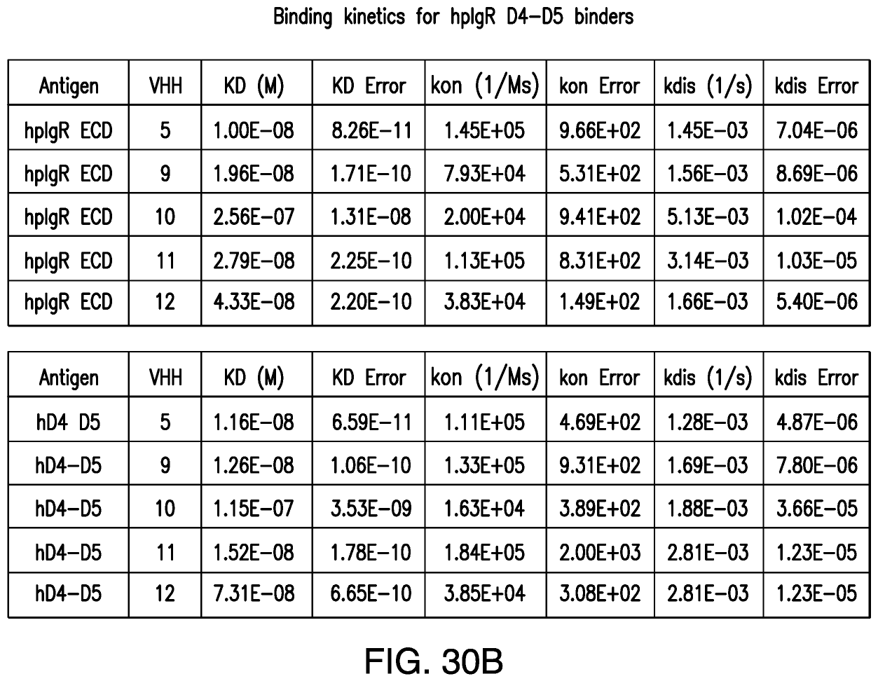

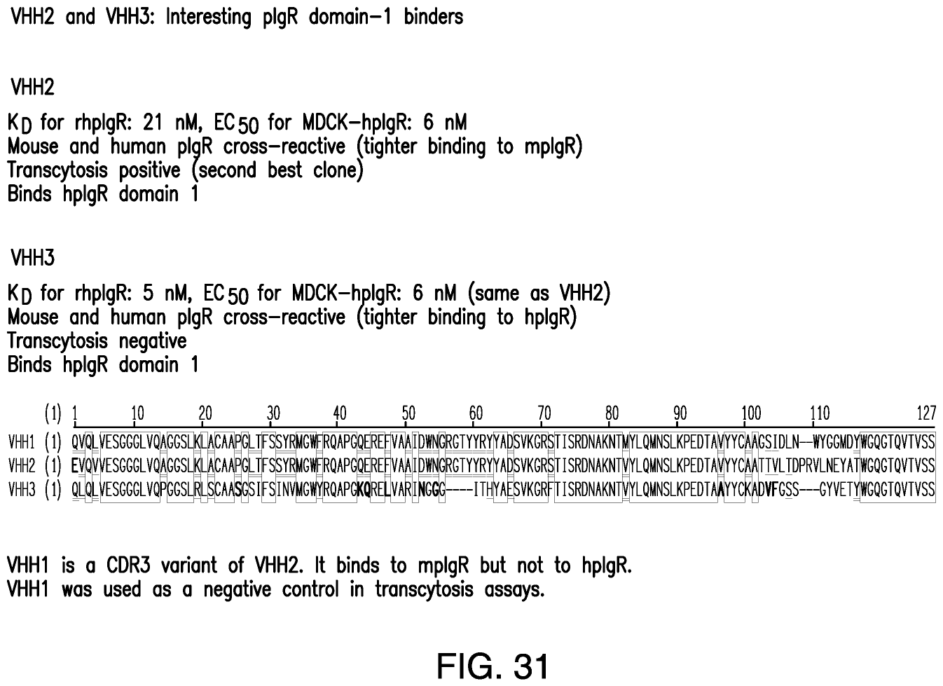

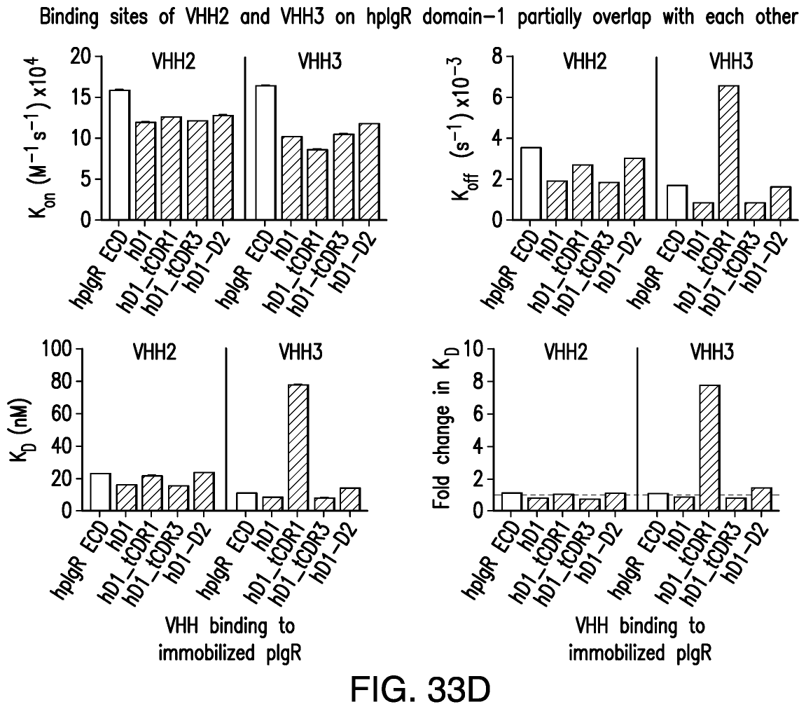

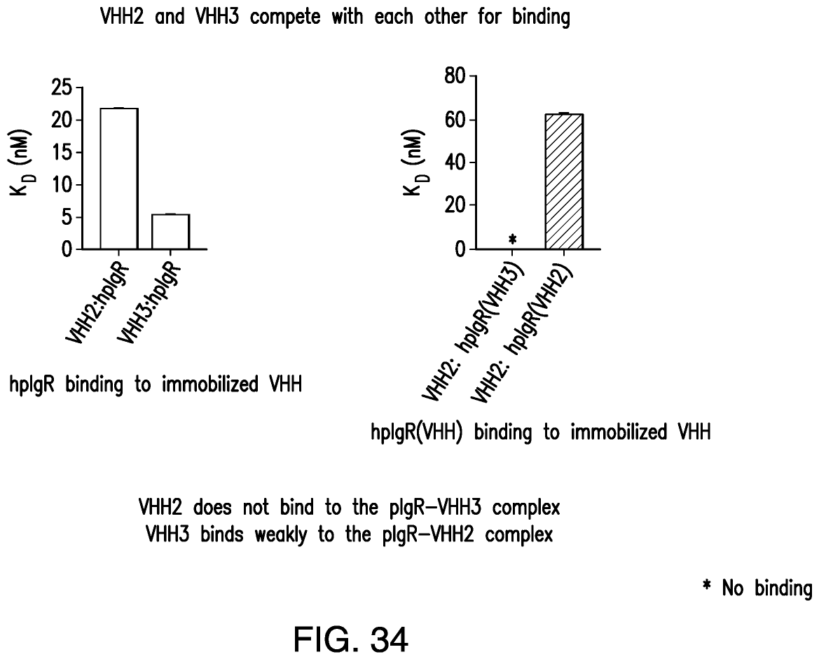

In Table 3, the K_{D} values for two VHH-mono-Fc molecules (VHH2 and VHH3) binding to six HIS-tagged pIgR constructs. VHH2 and VHH3 showed similar binding profiles towards CDR2 and CDR3 of hpIgR domain-1, while having different binding profiles towards CDR1 of hpIgR domain-1. The properties of VHH2 and VHH3 are summarized in FIG. 31. The data of FIG. 34 show that VHH2 and VHH3 compete with each other for binding to hpIgR.

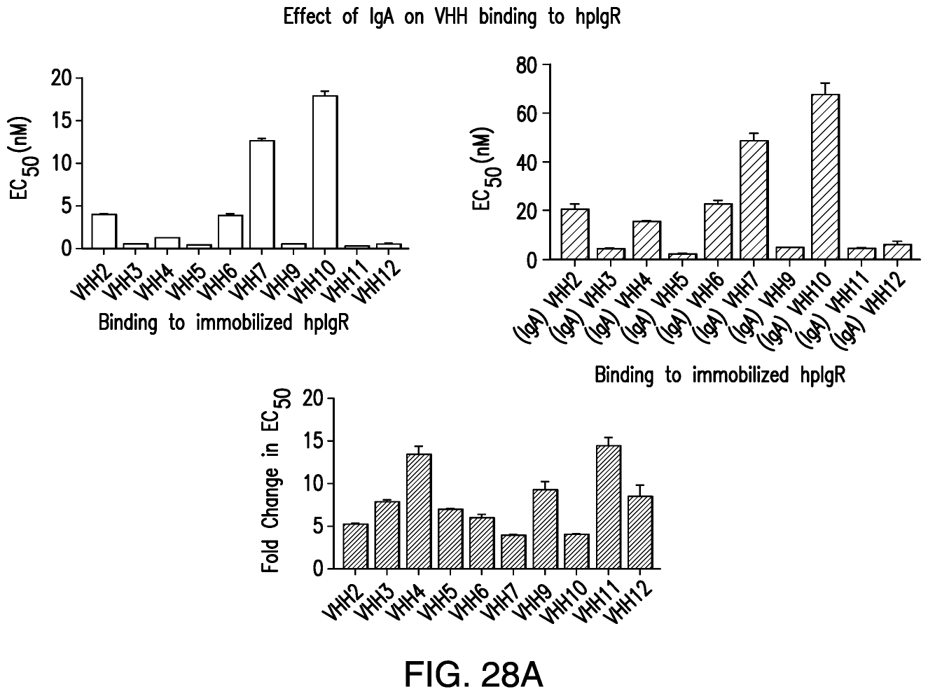

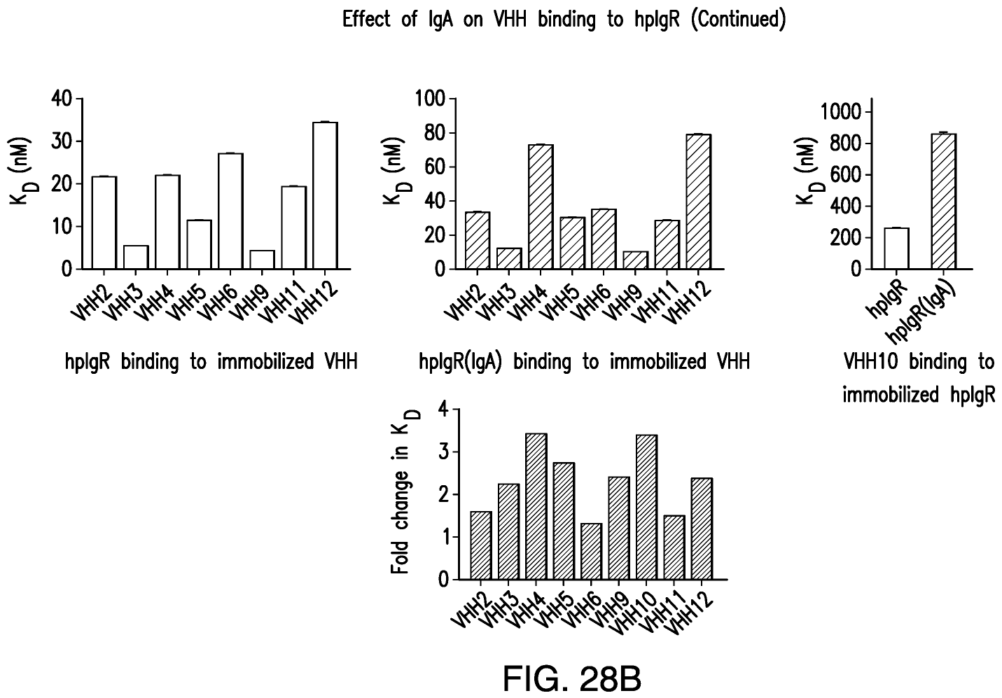

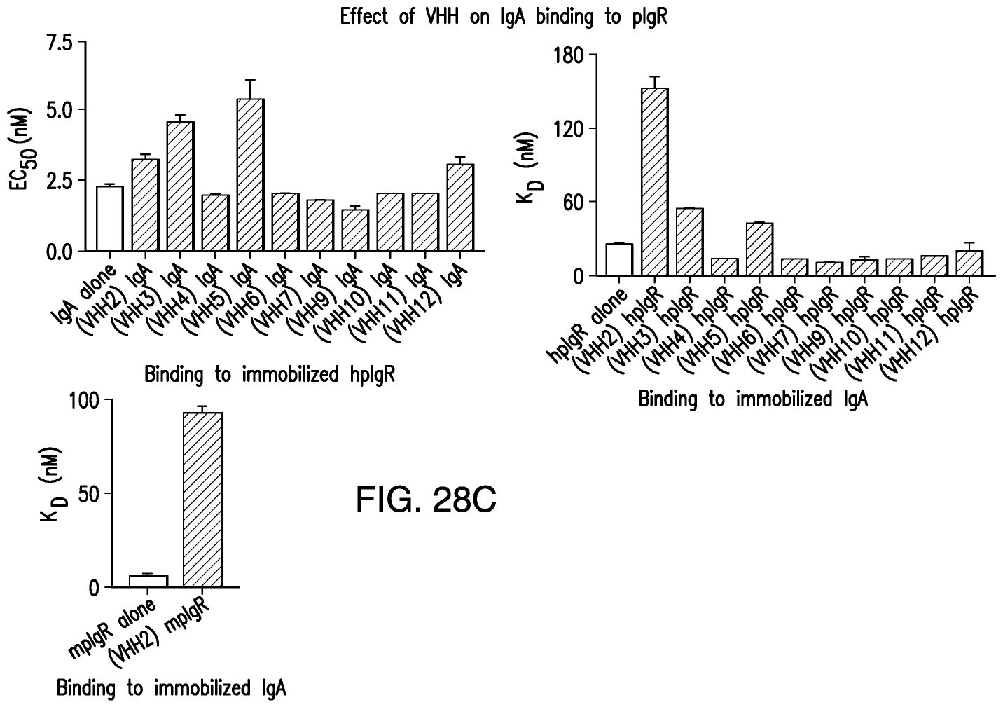

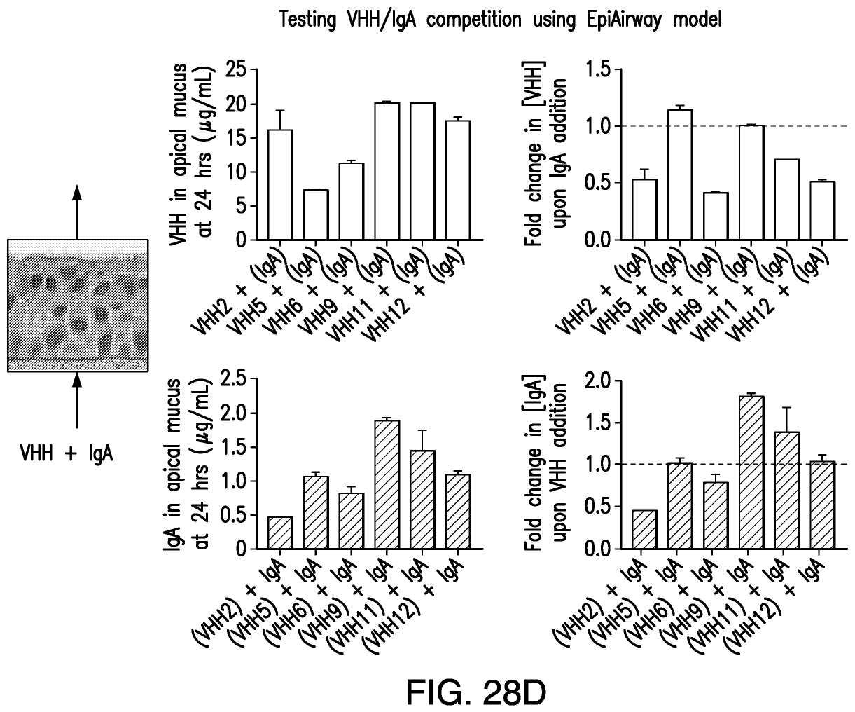

Competition binding assays showed that IgA had a negative effect on binding of VHH molecules to pIgR, possibly due to steric hindrance arising from the size difference between dimeric IgA and VHH. The data is shown in FIGS. 28A-28D. Given that hpIgR domain-1 is necessary for IgA binding, only VHH2 (domain-1 binder) had a negative effect on IgA binding to hpIgR and showed direct competition with IgA.

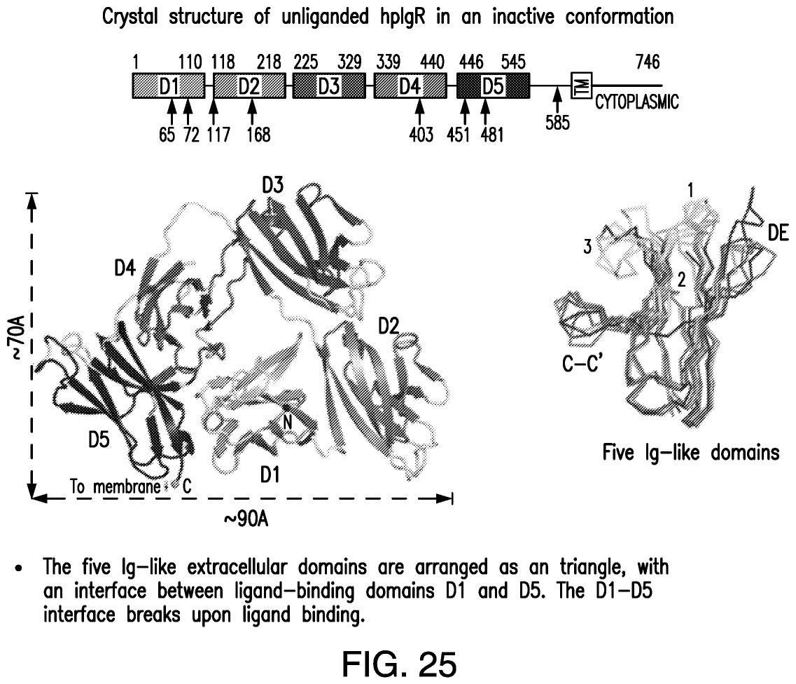

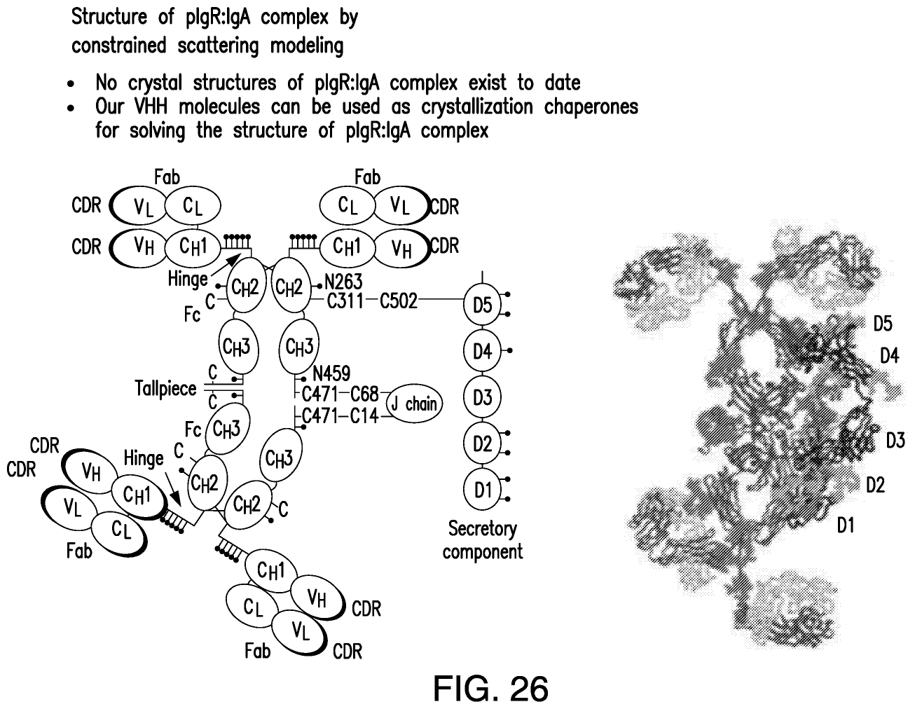

VHH2 and VHH3 showed similar binding profiles towards CDR2 and CDR3 of hpIgR domain-1, whereas showed different binding profiles towards CDR1 of hpIgR domain-1. This indicated that VHH2 and VHH3 overlap partial epitopes on domain-1 and thus competed with one another for binding to hpIgR. Further, binding assays suggested that VHH3 binds to a more hidden epitope on domain-1 relative to VHH2 (Table 2). Interestingly, VHH3-treated EpiAirway tissue model retained more pIgR in the basolateral epithelium relative to VHH2 or no VHH (FIG. 5). Given that the domain-1 plays a crucial interface and role in the inactive to active transitioning of hpIgR, these results suggest that VHH3 binding could shift the pIgR equilibrium towards an inactive conformation. As shown in FIG. 25, the five Ig-like extracellular domains are arranged as a triangle, with an interface between ligand-binding domains D1 and D5. The D1-D5 interface breaks upon ligand binding. FIG. 26 shows structure of pIgR:IgA complex by constrained scattering modeling.

A summary of the properties of the tested VHH molecules is shown in Table 4 below.

TABLE 4

| | | | | | | pIgR | IgA | |
|---|---|---|---|---|---|---|---|---|
| | Tₘ | K_{D} | EC₅₀ | mpIgR | Buried | Epitope | Dir. | Transcytosis |
| VHH | (° C.) | (nM) | (nM) | binding? | epitope? | Domain | Comp? | (Fold Incr.) |
| VHH2 | 64.1 | 21 | 6.3 | Yes | No | 1 | Yes | 34.2 |
| VHH3 | 75.9 | 5 | 6.4 | Yes | Yes | 1 | Yes | 2.6 |
| VHH4 | 61.5 | 22 | 32.9 | No | Yes | 2 | No | 12.6 |
| VHH5 | 76.4 | 11 | 4.3 | No | Yes | 4-5 | Yes | 6.9 |
| VHH6 | 69.3 | 27 | 11.5 | No | Yes | 2 | No | 30.7 |
| VHH7 | 55.3 | 521 | 36.4 | No | No | 4-5 | No | 1.3 |
| VHH9 | 70.3 | 4 | 1.5 | No | No | 4-5 | No | 22.4 |
| VHH10 | 53.9 | 256 | 20.4 | No | No | 4-5 | No | 5.0 |
| VHH11 | 69.2 | 19 | 1.5 | No | No | 4-5 | No | 32.0 |
| VHH12 | 61.5 | 34 | 4.6 | No | No | 5 | No | 38.6 |

The above examples show the generation, screening and characterization of hpIgR-binding VHH molecules by biophysical and functional assays. VHH molecules showed varying degrees of affinity, species cross-reactivity, biophysical characteristics, epitope diversity, IgA competition profiles and transcytosis activity in a human lung tissue model.

### 6.7 Example 7: Additional Transcytosis Assays

MDCK cells expressing hpIgR as described in Example 3, are a relevant epithelia model system and were used to assay forward and reverse transcytosis activities of VHH-mono-Fc molecules.

MDCK cells expressing hpIgR were cultured in DMEM containing 10% FBS at 37° C. with 5% CO₂. To prepare monolayers of such cells (MDCK-hpIgR monolayers), 5×10⁵ cells were seeded on fibronectin- and collagen-treated Transwell™ permeable supports (Costar) containing 0.4 μm polyester membrane filter. The cells were then incubated for 3 days, serum starved for 2 hours and supplemented with DMEM containing 1% FBS (assay media). Basolateral and apical chambers contained 1.5 ml and 0.5 ml of assay media, respectively.

To test the forward transcytosis activity of VHH-mono-Fc molecules across the MDCK-hpIgR monolayers, 20 μg of test or control VHH-mono-Fc molecules were added to the basolateral chamber and 100 μl of media was collected from the basolateral and apical chambers at different time points following the addition of VHH-mono-Fc molecules (0, 4, 8, 12, 24, 36 and 48 hours).

To test the reverse transcytosis activity of VHH-mono-Fc molecules across the MDCK-hpIgR monolayers, 20 μg of test or control VHH-mono-Fc molecules were added to the apical chamber and 100 μl of media was collected from the basolateral and apical chambers at different time points following the addition of VHH-mono-Fc (0, 4, 8, 12, 24, 36 and 48 hours).

The amount of VHH-mono-Fc present in basolateral and apical media was quantified by electrochemiluminescence method. Streptavidin MSD plates were coated with a biotinylated anti-VHH antibody (2 μg/ml in PBS) for 1 hour at RT with 1000 rpm, washed 3× with PBT, incubated with blocking buffer for 1 hour at RT, incubated with VHH-mono-Fc containing media/mucus (at different dilutions) for 1 hour at RT with 1000 rpm, washed 3× with PBT, incubated with ruthenylated-anti-human-Fc antibody (2 μg/ml in PBS) for 1 hour at RT with 1000 rpm, washed 3× with PBT and read plates in 40 μl reading buffer using the MSD imager. The amount of VHH in basolateral and apical chambers were calculated by plotting ECLU values against VHH-mono-Fc standard curves in Prism (Graphpad).

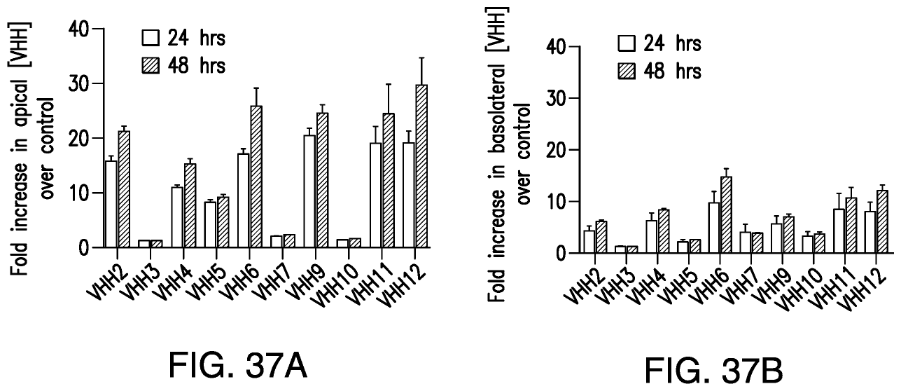

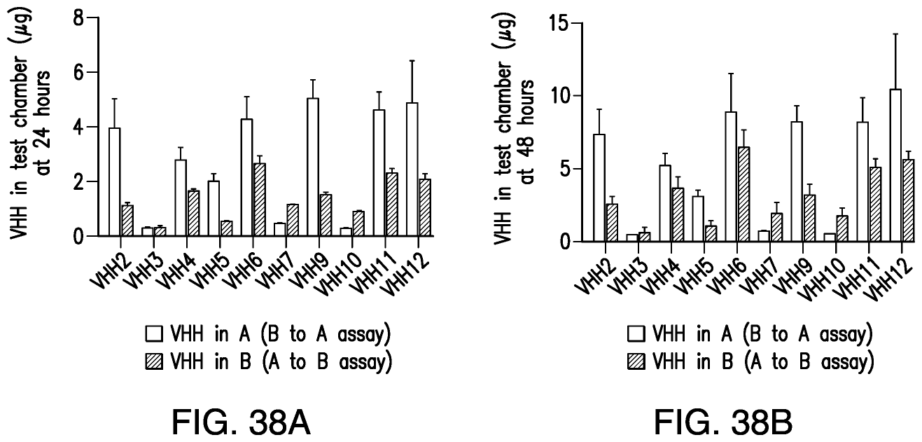

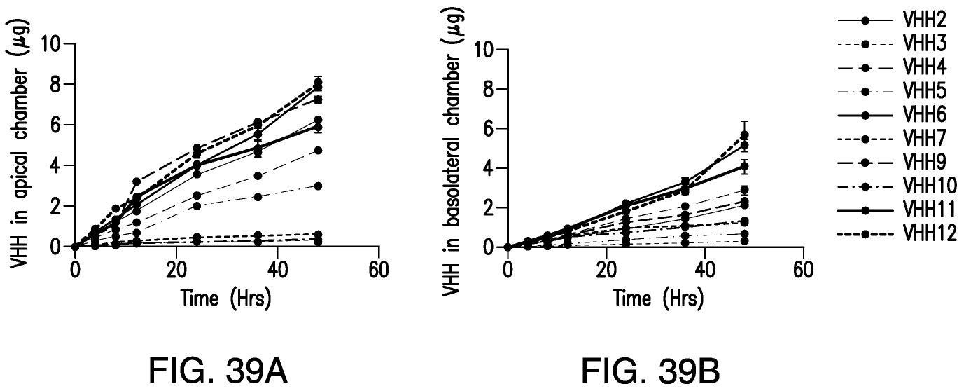

The results of the forward and reverse transcytosis assays are shown in FIGS. 37A, 37B, 38A, 38B, 39A and 39B.

A summary of the properties of the tested VHH molecules is shown in Table 5 below.

TABLE 5

| | | | | | | | For. | Rev. |
|---|---|---|---|---|---|---|---|---|
| | | | | | pIgR | IgA | Trans. | Trans |
| | Tₘ | K_{D} | EC₅₀ | mpIgR | Epitope | Dir. | (Fold | (Fold |
| VHH | (° C.) | (nM) | (nM) | binding? | Domain | Comp? | Incr.) | Incr.) |
| VHH2 | 64.1 | 21 | 6.3 | Yes | 1 | Yes | 21.0 | 5.7 |
| VHH3 | 75.9 | 5 | 6.4 | Yes | 1 | Yes | 1.0 | 1.0 |
| VHH4 | 61.5 | 22 | 32.9 | No | 2 | No | 15.1 | 8.0 |
| VHH5 | 76.4 | 11 | 4.3 | No | 4-5 | Yes | 8.9 | 2.1 |
| VHH6 | 69.3 | 27 | 11.5 | No | 2 | No | 25.6 | 14.6 |
| VHH7 | 55.3 | 521 | 36.4 | No | 4-5 | No | 1.9 | 3.3 |
| VHH9 | 70.3 | 4 | 1.5 | No | 4-5 | No | 24.2 | 6.6 |
| VHH10 | 53.9 | 256 | 20.4 | No | 4-5 | No | 1.3 | 3.4 |
| VHH11 | 69.2 | 19 | 1.5 | No | 4-5 | No | 24.2 | 10.3 |
| VHH12 | 61.5 | 34 | 4.6 | No | 5 | No | 29.5 | 11.7 |

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

SEQUENCE LISTING
VHH1 and VHH2 CDR1 (Kabat)
SEQ ID NO: 1
SYRMG
VHH3 CDR1 (Kabat)
SEQ ID NO: 2
INVMG
VHH4 CDR1 (Kabat)
SEQ ID NO: 3
SNAMG
VHH5 CDR1 (Kabat)
SEQ ID NO: 4
SYAMG
VHH6 CDR1 (Kabat)
SEQ ID NO: 5
SDAMG
VHH7 CDR1 (Kabat)
SEQ ID NO: 6
INVMG
VHH9 CDR1 (Kabat)
SEQ ID NO: 7
TYRMG
VHH10 CDR1 (Kabat)
SEQ ID NO: 8
RYAMG
VHH12 CDR1 (Kabat)
SEQ ID NO: 9
FNTYAMG
VHH1 and VHH2 CDR1 (Chothia)
SEQ ID NO: 10
GLTFSSY
VHH3 CDR1 (Chothia)
SEQ ID NO: 11
GSIFSIN
VHH4 CDR1 (Chothia)
SEQ ID NO: 12
GTSVSSN
VHH5 CDR1 (Chothia)
SEQ ID NO: 13
GRTFSSY
VHH6 CDR1 (Chothia)
SEQ ID NO: 14
GSSVSSD
VHH7 CDR1 (Chothia)
SEQ ID NO: 15
RSIGSIN
VHH9 CDR1 (Chothia)
SEQ ID NO: 16
GRTFSTY
VHH10 CDR1 (Chothia)
SEQ ID NO: 17
GFTFTRY
VHH11 CDR1 (Chothia)
SEQ ID NO: 18
GRTFTTY
VHH12 CDR1 (Chothia)
SEQ ID NO: 19
GRTLSFNTY
VHH1 and VHH2 CDR1 (IMGT)
SEQ ID NO: 20
GLTFSSYR
VHH3 CDR1 (IMGT)
SEQ ID NO: 21
GSIFSINV
VHH4 CDR1 (IMGT)
SEQ ID NO: 22
GTSVSSNA
VHH5 CDR1 (IMGT)
SEQ ID NO: 23
GRTFSSYA
VHH6 CDR1 (IMGT)
SEQ ID NO: 24
GSSVSSDA
VHH7 CDR1 (IMGT)
SEQ ID NO: 25
RSIGSINV
VHH9 CDR1 (IMGT)
SEQ ID NO: 26
GRTFSTYR
VHH10 CDR1 (IMGT)
SEQ ID NO: 27
GFTFTRYA
VHH11 CDR1 (IMGT)
SEQ ID NO: 28
GRTFTTYR
VHH12 CDR1 (IMGT)
SEQ ID NO: 29
GRTLSFNTYA
VHH1 and VHH2 CDR2 (Kabat)
SEQ ID NO: 30
AIDWNGRGTYYRYYADSVKG
VHH3 CDR2 (Kabat)
SEQ ID NO: 31
RINGGGITHYAESVKG
VHH4 CDR2 (Kabat)
SEQ ID NO: 32
FIDRIATTTIATSVKG
VHH5 CDR2 (Kabat)
SEQ ID NO: 33
AITWNGGTTYYADSVKG
VHH6 CDR2 (Kabat)
SEQ ID NO: 34
FISGGGTTTYADSVKG
VHH7 CDR2 (Kabat)
SEQ ID NO: 35
RITGGGSTHYAESVKG
VHH9 CDR2 (Kabat)
SEQ ID NO: 36
AISWSGGSTTYADPVKG
VHH10 CDR2 (Kabat)
SEQ ID NO: 37
AISWSGSSAGYGDSVKG
VHH11 CDR2 (Kabat)
SEQ ID NO: 38
AIRWSGGRTLYADSVKG
VHH12 CDR2 (Kabat)
SEQ ID NO: 39
SITWNGGSTSYADSVKG
VHH1 and VHH2 CDR2 (Chothia)
SEQ ID NO: 40
DWNGRGTYY
VHH3 CDR2 (Chothia)
SEQ ID NO: 41
NGGGI
VHH4 CDR2 (Chothia)
SEQ ID NO: 42
DRIAT
VHH5 CDR2 (Chothia)
SEQ ID NO: 43
TWNGGT
VHH6 CDR2 (Chothia)
SEQ ID NO: 44
SGGGT
VHH7 CDR2 (Chothia)
SEQ ID NO: 45
TGGGS
VHH9 CDR2 (Chothia)
SEQ ID NO: 46
SWSGGS
VHH10 CDR2 (Chothia)
SEQ ID NO: 47
SWSGSS
VHH11 CDR2 (Chothia)
SEQ ID NO: 48
RWSGGR
VHH12 CDR2 (Chothia)
SEQ ID NO: 49
TWNGGS
VHH1 and VHH2 CDR2 (IMGT)
SEQ ID NO: 50
IDWNGRGTYY
VHH3 CDR2 (IMGT)
SEQ ID NO: 51
INGGGIT
VHH4 CDR2 (IMGT)
SEQ ID NO: 52
IDRIATT
VHH5 CDR2 (IMGT)
SEQ ID NO: 53
ITWNGGTT
VHH6 CDR2 (IMGT)
SEQ ID NO: 54
ISGGGTT
VHH7 CDR2 (IMGT)
SEQ ID NO: 55
ITGGGST
VHH9 CDR2 (IMGT)
SEQ ID NO: 56
ISWSGGST
VHH10 CDR2 (IMGT)
SEQ ID NO: 57
ISWSGSSA
VHH11 CDR2 (IMGT)
SEQ ID NO: 58
IRWSGGRT
VHH12 CDR2 (IMGT)
SEQ ID NO: 59
ITWNGGST
VHH1 CDR3 (Kabat)
SEQ ID NO: 60
GSIDLNWYGGMDY
VHH2 CDR3 (Kabat)
SEQ ID NO: 61
TTVLTDPRVLNEYAT
VHH3 CDR3 (Kabat)
SEQ ID NO: 62
DVFGSSGYVETY
VHH4 CDR3 (Kabat)
SEQ ID NO: 63
PLTAR
VHH5 CDR3 (Kabat)
SEQ ID NO: 64
DPFNQGY
VHH6 CDR3 (Kabat)
SEQ ID NO: 65
PLTSR
VHH7 CDR3 (Kabat)
SEQ ID NO: 66
MVNPIITAWGTIGVREIPDYDY
VHH9 CDR3 (Kabat)
SEQ ID NO: 67
DQRGY
VHH10 CDR3 (Kabat)
SEQ ID NO: 68
DPFNQGY
VHH11 CDR3 (Kabat)
SEQ ID NO: 69
DLAEYSGTYSSPADSPAGYDY
VHH12 CDR3 (Kabat)
SEQ ID NO: 70
ARYYVSGTYFPANY
VHH1 CDR3 (Chothia)
SEQ ID NO: 71
GSIDLNWYGGMDY
VHH2 CDR3 (Chothia)
SEQ ID NO: 72
TTVLTDPRVLNEYAT
VHH3 CDR3 (Chothia)
SEQ ID NO: 73
DVFGSSGYVETY
VHH4 CDR3 (Chothia)
SEQ ID NO: 74
PLTAR
VHH5 CDR3 (Chothia)
SEQ ID NO: 75
DPFNQGY
VHH6 CDR3 (Chothia)
SEQ ID NO: 76
PLTSR
VHH7 CDR3 (Chothia)
SEQ ID NO: 77
MVNPIITAWGTIGVREIPDYDY
VHH9 CDR3 (Chothia)
SEQ ID NO: 78
DQRGY
VHH10 CDR3 (Chothia)
SEQ ID NO: 79
DPFNQGY
VHH11 CDR3 (Chothia)
SEQ ID NO: 80
DLAEYSGTYSSPADSPAGYDY
VHH12 CDR3 (Chothia)
SEQ ID NO: 81
ARYYVSGTYFPANY
VHH1 CDR3 (IMGT)
SEQ ID NO: 82
CAAGSIDLNWYGGMDY
VHH2 CDR3 (IMGT)
SEQ ID NO: 83
CAATTVLTDPRVLNEYAT
VHH3 CDR3 (IMGT)
SEQ ID NO: 84
KADVFGSSGYVETY
VHH4 CDR3 (IMGT)
SEQ ID NO: 85
NHPLTAR
VHH5 CDR3 (IMGT)
SEQ ID NO: 86
AADPFNQGY
VHH6 CDR3 (IMGT)
SEQ ID NO: 87
NHPLTSR
VHH7 CDR3 (IMGT)
SEQ ID NO: 88
ASMVNPIITAWGTIGVREIPDYDY
VHH9 CDR3 (IMGT)
SEQ ID NO: 89
NDQRGY
VHH10 CDR3 (IMGT)
SEQ ID NO: 90
AADPFNQGY
VHH11 CDR3 (IMGT)
SEQ ID NO: 91
AADLAEYSGTYSSPADSPAGYDY
VHH12 CDR3 (IMGT)
SEQ ID NO: 92
AAARYYVSGTYFPANY
VHH1 - VH amino acid sequence
SEQ ID NO: 93
QVQLVESGGGLVQAGGSLKLACAAPGLTFSSYRMGWFRQAPGQEREFVAA
IDWNGRGTYYRYYADSVKGRSTISRDNAKNTMYLQMNSLKPEDTAVYYCA
AGSIDLNWYGGMDYWGQGTQVTVSS
VHH2 - VH amino acid sequence
SEQ ID NO: 94
EVQVVESGGGLVQAGGSLKLACAAPGLTFSSYRMGWFRQAPGQEREFVAA
IDWNGRGTYYRYYADSVKGRSTISRDNAKNTVYLQMNSLKPEDTAVYYCA
ATTVLTDPRVLNEYATWGQGTQVTVSS
VHH3 - VH amino acid sequence
SEQ ID NO: 95
QLQLVESGGGLVQPGGSLRLSCAASGSIFSINVMGWYRQAPGKQRELVAR
INGGGITHYAESVKGRFTISRDNAKNTVYLQMNSLKPEDTAAYYCKADVF
GSSGYVETYWGQGTQVTVSS
VHH4 - VH amino acid sequence
SEQ ID NO: 96
EVQVVESGGGLVQAGGSLRLSCAVSGTSVSSNAMGWYRQAPGKQREWVGF
IDRIATTTIATSVKGRFAITRDNAKNTVYLQMSGLKPEDTAVYYCNHPLT
ARWGQGTQVTVSS
VHH5 - VH amino acid sequence
SEQ ID NO: 97
QVQLVESGGGLVQAGGSLRLSCAASGRTFSSYAMGWFRQAPGKEREFVAA
ITWNGGTTYYADSVKGRFTISRDNAKNTVYLQMNSLKPEDTAVYYCAADP
FNQGYWGQGTQVTVSS
VHH6 - VH amino acid sequence
SEQ ID NO: 98
EVQLVESGGGLVQAGGSLRLSCAVSGSSVSSDAMGWYRQAPGNQRAWVAF
ISGGGTTTYADSVKGRFTISRDNTKNTVYLHMNSLKPEDTAVYYCNHPLT
SRWGQGTQVTVSS
VHH7 - VH amino acid sequence
SEQ ID NO: 99
EVQVVESGGGLVQAGGSLRLACVASRSIGSINVMGWYRQAPGKQRDLVAR
ITGGGSTHYAESVKGRFTISRDNAKNTVYLQMNSLEPEDTAVYYCASMVN
PIITAWGTIGVREIPDYDYWGQGTQVTVSS
VHH9 - VH amino acid sequence
SEQ ID NO: 100
QVQLVESGGGLVQAGGSLRLSCAVSGRTFSTYRMGWFRQAPGKERSFVAA
ISWSGGSTTYADPVKGRFTISRDNAKNTVYLRMNSLKPEDTAVYYCNDQR
GYWGQGTLVTVSS
VHH10 - VH amino acid sequence
SEQ ID NO: 101
EVQVVESGGGLVQAGGSLRLSCAASGFTFTRYAMGWFRQAPGKERSFVAA
ISWSGSSAGYGDSVKGRFTISRDNAKNTLYLQMNSLKPEDTAVYYCAADP
FNQGYWGQGTQVTVSS
VHH11 - VH amino acid sequence
SEQ ID NO: 102
EVQVVESGGGLVQAGGSLRLSCAASGRTFTTYRMGWFRQAPGKEREFVAA
IRWSGGRTLYADSVKGRFTISRDNAKNTAYLQMNNLRPEDTAVYYCAADL
AEYSGTYSSPADSPAGYDYWGQGTQVTVSS
VHH12 - VH amino acid sequence
SEQ ID NO: 103
QVQLVETGGGLVQAGDSLRLSCAASGRTLSFNTYAMGWFRQAPGKEREFV
ASITWNGGSTSYADSVKGRFTITRDNAKNTATLRMNSLQPDDTAVYYCAA
ARYYVSGTYFPANYWGQGTQVTVSS
VHH1 - linker-mono-Fc protein
SEQ ID NO: 104
QVQLVESGGGLVQAGGSLKLACAAPGLTFSSYRMGWFRQAPGQEREFVAA
IDWNGRGTYYRYYADSVKGRSTISRDNAKNTMYLQMNSLKPEDTAVYYCA
AGSIDLNWYGGMDYWGQGTQVTVSSEPKTPKPQPQPQLQPQPNPTTESKS
PKSPAPELLGGPSVFLFPPKPKDTLMISRTPEVTCVVVDVSHEDPEVKFN
WYVDGVEVHNAKTKPREEQYNSTYRVVSVLTVLHQDWLNGKEYKCKVSNK
ALPAPIEKTISKAKGQPREPQVYTKPPSREEMTKNQVSLSCLVKGFYPSD
IAVEWESNGQPENNYKTTVPVLDSDGSFRLASYLTVDKSRWQQGNVFSCS
VMHEALHNHYTQKSLSLSPGK
VHH2 - linker-mono-Fc protein
SEQ ID NO: 105
EVQVVESGGGLVQAGGSLKLACAAPGLTFSSYRMGWFRQAPGQEREFVAA
IDWNGRGTYYRYYADSVKGRSTISRDNAKNTVYLQMNSLKPEDTAVYYCA
ATTVLTDPRVLNEYATWGQGTQVTVSSEPKTPKPQPQPQLQPQPNPTTES
KSPKSPAPELLGGPSVFLFPPKPKDTLMISRTPEVTCVVVDVSHEDPEVK
FNWYVDGVEVHNAKTKPREEQYNSTYRVVSVLTVLHQDWLNGKEYKCKVS
NKALPAPIEKTISKAKGQPREPQVYTKPPSREEMTKNQVSLSCLVKGFYP
SDIAVEWESNGQPENNYKTTVPVLDSDGSFRLASYLTVDKSRWQQGNVFS
CSVMHEALHNHYTQKSLSLSPGK
VHH3 - linker-mono-Fc protein
SEQ ID NO: 106
QLQLVESGGGLVQPGGSLRLSCAASGSIFSINVMGWYRQAPGKQRELVAR
INGGGITHYAESVKGRFTISRDNAKNTVYLQMNSLKPEDTAAYYCKADVF
GSSGYVETYWGQGTQVTVSSEPKTPKPQPQPQLQPQPNPTTESKSPKSPA
PELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAP
IEKTISKAKGQPREPQVYTKPPSREEMTKNQVSLSCLVKGFYPSDIAVEW
ESNGQPENNYKTTVPVLDSDGSFRLASYLTVDKSRWQQGNVFSCSVMHEA
LHNHYTQKSLSLSPGK
VHH4 - linker-mono-Fc protein
SEQ ID NO: 107
EVQVVESGGGLVQAGGSLRLSCAVSGTSVSSNAMGWYRQAPGKQREWVGF
IDRIATTTIATSVKGRFAITRDNAKNTVYLQMSGLKPEDTAVYYCXHPXT
ARWGQGTQVTVSSEPKTPKPQPQPQLQPQPNPTTESKSPKSPAPELLGGP
SVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISK
AKGQPREPQVYTKPPSREEMTKNQVSLSCLVKGFYPSDIAVEWESNGQPE
NNYKTTVPVLDSDGSFRLASYLTVDKSRWQQGNVFSCSVMHEALHNHYTQ
KSLSLSPGK
VHH5 - linker-mono-Fc protein
SEQ ID NO: 108
QVQLVESGGGLVQAGGSLRLSCAASGRTFSSYAMGWFRQAPGKEREFVAA
ITWNGGTTYYADSVKGRFTISRDNAKNTVYLQMNSLKPEDTAVYYCAADP
FNQGYWGQGTQVTVSSEPKTPKPQPQPQLQPQPNPTTESKSPKSPAPELL
GGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKT
ISKAKGQPREPQVYTKPPSREEMTKNQVSLSCLVKGFYPSDIAVEWESNG
QPENNYKTTVPVLDSDGSFRLASYLTVDKSRWQQGNVFSCSVMHEALHNH
YTQKSLSLSPGK
VHH6 - linker-mono-Fc protein
SEQ ID NO: 109
EVQLVESGGGLVQAGGSLRLSCAVSGSSVSSDAMGWYRQAPGNQRAWVAF
ISGGGTTTYADSVKGRFTISRDNTKNTVYLHMNSLKPEDTAVYYCNHPLT
SRWGQGTQVTVSSEPKTPKPQPQPQLQPQPNPTTESKSPKSPAPELLGGP
SVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISK
AKGQPREPQVYTKPPSREEMTKNQVSLSCLVKGFYPSDIAVEWESNGQPE
NNYKTTVPVLDSDGSFRLASYLTVDKSRWQQGNVFSCSVMHEALHNHYTQ
KSLSLSPGK
VHH7 - linker-mono-Fc protein
SEQ ID NO: 110
EVQVVESGGGLVQAGGSLRLACVASRSIGSINVMGWYRQAPGKQRDLVAR
ITGGGSTHYAESVKGRFTISRDNAKNTVYLQMNSLEPEDTAVYYCASMVN
PIITAWGTIGVREIPDYDYWGQGTQVTVSSEPKTPKPQPQPQLQPQPNPT
TESKSPKSPAPELLGGPSVFLFPPKPKDTLMISRTPEVTCVVVDVSHEDP
EVKFNWYVDGVEVHNAKTKPREEQYNSTYRVVSVLTVLHQDWLNGKEYKC
KVSNKALPAPIEKTISKAKGQPREPQVYTKPPSREEMTKNQVSLSCLVKG
FYPSDIAVEWESNGQPENNYKTTVPVLDSDGSFRLASYLTVDKSRWQQGN
VFSCSVMHEALHNHYTQKSLSLSPGK
VHH9 - linker-mono-Fc protein
SEQ ID NO: 111
QVQLVESGGGLVQAGGSLRLSCAVSGRTFSTYRMGWFRQAPGKERSFVAA
ISWSGGSTTYADPVKGRFTISRDNAKNTVYLRMNSLKPEDTAVYYCNDQR
GYWGQGTLVTVSSEPKTPKPQPQPQLQPQPNPTTESKSPKSPAPELLGGP
SVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISK
AKGQPREPQVYTKPPSREEMTKNQVSLSCLVKGFYPSDIAVEWESNGQPE
NNYKTTVPVLDSDGSFRLASYLTVDKSRWQQGNVFSCSVMHEALHNHYTQ
KSLSLSPGK
VHH10 - linker-mono-Fc protein
SEQ ID NO: 112
EVQVVESGGGLVQAGGSLRLSCAASGFTFTRYAMGWFRQAPGKERSFVAA
ISWSGSSAGYGDSVKGRFTISRDNAKNTLYLQMNSLKPEDTAVYYCAADP
FNQGYWGQGTQVTVSSEPKTPKPQPQPQLQPQPNPTTESKSPKSPAPELL
GGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKT
ISKAKGQPREPQVYTKPPSREEMTKNQVSLSCLVKGFYPSDIAVEWESNG
QPENNYKTTVPVLDSDGSFRLASYLTVDKSRWQQGNVFSCSVMHEALHNH
YTQKSLSLSPGK
VHH11 - linker-mono-Fc protein
SEQ ID NO: 113
EVQVVESGGGLVQAGGSLRLSCAASGRTFTTYRMGWFRQAPGKEREFVAA
IRWSGGRTLYADSVKGRFTISRDNAKNTAYLQMNNLRPEDTAVYYCAADL
AEYSGTYSSPADSPAGYDYWGQGTQVTVSSEPKTPKPQPQPQLQPQPNPT
TESKSPKSPAPELLGGPSVFLFPPKPKDTLMISRTPEVTCVVVDVSHEDP
EVKFNWYVDGVEVHNAKTKPREEQYNSTYRVVSVLTVLHQDWLNGKEYKC
KVSNKALPAPIEKTISKAKGQPREPQVYTKPPSREEMTKNQVSLSCLVKG
FYPSDIAVEWESNGQPENNYKTTVPVLDSDGSFRLASYLTVDKSRWQQGN
VFSCSVMHEALHNHYTQKSLSLSPGK
VHH12 - linker-mono-Fc protein
SEQ ID NO: 114
QVQLVETGGGLVQAGDSLRLSCAASGRTLSFNTYAMGWFRQAPGKEREFV
ASITWNGGSTSYADSVKGRFTITRDNAKNTATLRMNSLQPDDTAVYYCAA
ARYYVSGTYFPANYWGQGTQVTVSSEPKTPKPQPQPQLQPQPNPTTESKS
PKSPAPELLGGPSVFLFPPKPKDTLMISRTPEVTCVVVDVSHEDPEVKFN
WYVDGVEVHNAKTKPREEQYNSTYRVVSVLTVLHQDWLNGKEYKCKVSNK
ALPAPIEKTISKAKGQPREPQVYTKPPSREEMTKNQVSLSCLVKGFYPSD
IAVEWESNGQPENNYKTTVPVLDSDGSFRLASYLTVDKSRWQQGNVFSCS
VMHEALHNHYTQKSLSLSPGK
IgA Heavy Chain
SEQ ID NO: 115
QVQLVQSGAEVKKPGSSVKVSCKSSGGTSNNYAISWVRQAPGQGLDWMGG
ISPIFGSTAYAQKFQGRVTISADIFSNTAYMELNSLTSEDTAVYFCARHG
NYYYYSGMDVWGQGTTVTVSSASPTSPKVFPLSLDSTPQDGNVVVACLVQ
GFFPQEPLSVTWSESGQNVTARNFPPSQDASGDLYTTSSQLTLPATQCPD
GKSVTCHVKHYTNPSQDVTVPCPVPPPPPCCHPRLSLHRPALEDLLLGSE
ANLTCTLTGLRDASGATFTWTPSSGKSAVQGPPERDLCGCYSVSSVLPGC
AQPWNHGETFTCTAAHPELKTPLTANITKSGNTFRPEVHLLPPPSEELAL
NELVTLTCLARGFSPKDVLVRWLQGSQELPREKYLTWASRQEPSQGTTTF
AVTSILRVAAEDWKKGDTFSCMVGHEALPLAFTQKTIDRLAGKPTHVNVS
VVMAEVDGTCY
IgA Light Chain
SEQ ID NO: 116
QSALTQPPAVSGTPGQRVTISCSGSDSNIGRRSVNWYQQFPGTAPKLLIY
SNDQRPSVVPDRFSGSKSGTSASLAISGLQSEDEAEYYCAAWDDSLKGAV
FGGGTQLTVLGQPKAAPSVTLFPPSSEELQANKATLVCLISDFYPGAVTV
AWKADSSPVKAGVETTTPSKQSNNKYAASSYLSLTPEQWKSHRSYSCQVT
HEGSTVEKTVAPTECS
IgA J Chain
SEQ ID NO: 117
SRDSSASASRVAGITAQEDERIVLVDNKCKCARITSRIIRSSEDPNEDIV
ERNIRIIVPLNNRENISDPTSPLRTRFVYHLSDLCKKCDPTEVELDNQIV
TATQSNICDEDSATETCYTYDRNKCYTAVVPLVYGGETKMVETALTPDAC
YPD
human pIgR extracellular domain (ECD)
SEQ ID NO: 118
KSPIFGPEEVNSVEGNSVSITCYYPPTSVNRHTRKYWCRQGARGGCITLI
SSEGYVSSKYAGRANLTNFPENGTFVVNIAQLSQDDSGRYKCGLGINSRG
LSFDVSLEVSQGPGLLNDTKVYTVDLGRTVTINCPFKTENAQKRKSLYKQ
IGLYPVLVIDSSGYVNPNYTGRIRLDIQGTGQLLFSVVINQLRLSDAGQY
LCQAGDDSNSNKKNADLQVLKPEPELVYEDLRGSVTFHCALGPEVANVAK
FLCRQSSGENCDVVVNTLGKRAPAFEGRILLNPQDKDGSFSVVITGLRKE
DAGRYLCGAHSDGQLQEGSPIQAWQLFVNEESTIPRSPTVVKGVAGSSVA
VLCPYNRKESKSIKYWCLWEGAQNGRCPLLVDSEGWVKAQYEGRLSLLEE
PGNGTFTVILNQLTSRDAGFYWCLTNGDTLWRTTVEIKIIEGEPNLKVPG
NVTAVLGETLKVPCHFPCKFSSYEKYWCKWNNTGCQALPSQDEGPSKAFV
NCDENSRLVSLTLNLVTRADEGWYWCGVKQGHFYGETAAVYVAVEERKAA
GSRDVSLAKADAAPDEKVLDSGFREIENKAIQDPRLFAEEKAVADTRDQA
DGSRASVDSGSSEEQGGSSRHHHHHH
human pIgR extracellular domain 1 (D1)
SEQ ID NO: 119
KSPIFGPEEVNSVEGNSVSITCYYPPTSVNRHTRKYWCRQGARGGCITLI
SSEGYVSSKYAGRANLTNFPENGTFVVNIAQLSQDDSGRYKCGLGINSRG
LSFDVSLEVGSHHHHHH
human pIgR extracellular domain 2 (D2)
SEQ ID NO: 120
SQGPGLLNDTKVYTVDLGRTVTINCPFKTENAQKRKSLYKQIGLYPVLVI
DSSGYVNPNYTGRIRLDIQGTGQLLFSVVINQLRLSDAGQYLCQAGDDSN
SNKKNADLQVLKPEPGSHHHHHH
human pIgR extracellular domain 3 (D3)
SEQ ID NO: 121
KPEPELVYEDLRGSVTFHCALGPEVANVAKFLCRQSSGENCDVVVNTLGK
RAPAFEGRILLNPQDKDGSFSVVITGLRKEDAGRYLCGAHSDGQLQEGSP
IQAWQLFVNEESTGSHHHHHH
human pIgR extracellular domain 5 (D5)
SEQ ID NO: 122
GEPNLKVPGNVTAVLGETLKVPCHFPCKFSSYEKYWCKWNNTGCQALPSQ
DEGPSKAFVNCDENSRLVSLTLNLVTRADEGWYWCGVKQGHFYGETAAVY
VAVEERGSHHHHHH
human pIgR extracellular domain 1-
domain 2 (D1-D2)
SEQ ID NO: 123
KSPIFGPEEVNSVEGNSVSITCYYPPTSVNRHTRKYWCRQGARGGCITLI
SSEGYVSSKYAGRANLTNFPENGTFVVNIAQLSQDDSGRYKCGLGINSRG
LSFDVSLEVSQGPGLLNDTKVYTVDLGRTVTINCPFKTENAQKRKSLYKQ
IGLYPVLVIDSSGYVNPNYTGRIRLDIQGTGQLLFSVVINQLRLSDAGQY
LCQAGDDSNSNKKNADLQVLKPGSHHHHHH
human pIgR extracellular domain 2-
domain 3 (D2-D3)
SEQ ID NO: 124
SQGPGLLNDTKVYTVDLGRTVTINCPFKTENAQKRKSLYKQIGLYPVLVI
DSSGYVNPNYTGRIRLDIQGTGQLLFSVVINQLRLSDAGQYLCQAGDDSN
SNKKNADLQVLKPEPELVYEDLRGSVTFHCALGPEVANVAKFLCRQSSGE
NCDVVVNTLGKRAPAFEGRILLNPQDKDGSFSVVITGLRKEDAGRYLCGA
HSDGQLQEGSPIQAWQLFVNGSHHHHHH
human pIgR extracellular domain 4-
domain 5 (D4-D5)
SEQ ID NO: 125
STIPRSPTVVKGVAGSSVAVLCPYNRKESKSIKYWCLWEGAQNGRCPLLV
DSEGWVKAQYEGRLSLLEEPGNGTFTVILNQLTSRDAGFYWCLTNGDTLW
RTTVEIKIIEGEPNLKVPGNVTAVLGETLKVPCHFPCKFSSYEKYWCKWN
NTGCQALPSQDEGPSKAFVNCDENSRLVSLTLNLVTRADEGWYWCGVKQG
HFYGETAAVYVAVEERGSHHHHHH
pIgR CDR1 of D1
SEQ ID NO: 126
GPQYASY
pIgR CDR2 of D1
SEQ ID NO: 127
DAP
pIgR CDR3 of D1
SEQ ID NO: 128
VGGVWSAD
mouse pIgR extracellular domain (ECD)
SEQ ID NO: 129
KSPIFGPQEVSSIEGDSVSITCYYPDTSVNRHTRKYWCRQGASGMCTTLI
SSNGYLSKEYSGRANLINFPENNTFVINIEQLTQDDTGSYKCGLGTSNRG
LSFDVSLEVSQVPELPSDTHVYTKDIGRNVTIECPFKRENAPSKKSLCKK
TNQSCELVIDSTEKVNPSYIGRAKLFMKGTDLTVFYVNISHLTHNDAGLY
ICQAGEGPSADKKNVDLQVLAPEPELLYKDLRSSVTFECDLGREVANEAK
YLCRMNKETCDVIINTLGKRDPDFEGRILITPKDDNGRFSVLITGLRKED
AGHYQCGAHSSGLPQEGWPIQTWQLFVNEESTIPNRRSVVKGVTGGSVAI
ACPYNPKESSSLKYWCRWEGDGNGHCPVLVGTQAQVQEEYEGRLALFDQP
GNGTYTVILNQLTTEDAGFYWCLTNGDSRWRTTIELQVAEATREPNLEVT
PQNATAVLGETFTVSCHYPCKFYSQEKYWCKWSNKGCHILPSHDEGARQS
SVSCDQSSQLVSMTLNPVSKEDEGWYWCGVKQGQTYGETTAIYIAVEERT
RGSSHVNPTDANARAKVALEEEVVDSSISEKENKAIPNPGPFANEREIQN
VGDQAQENRASGDAGSADGQSRSSSSKHHHHHH
hinge region (AA)
SEQ ID NO: 130
EPKTPKPQPQPQLQPQPNPTTESKSPK
hinge region (DNA)
SEQ ID NO: 131
Gaacccaagacaccaaaaccacaaccacaaccacaactacaaccacaacc
caatcctacaacagaatccaagagccccaaaa
human IgG1 Mono-Fc DNA sequence
SEQ ID NO: 132
Agcccagcacctgaactcctggggggaccgtcagtcttcctcttcccccc
aaaacccaaggacaccctcatgatctcccggacccctgaggtcacatgcg
tggtggtggacgtgagccacgaagaccctgaggtcaagttcaactggtac
gtggacggcgtggaggtgcataatgccaagacaaagccgcgggaggagca
gtacaacagcacgtaccgtgtggtcagcgtcctcaccgtcctgcaccagg
actggctgaatggcaaggagtacaagtgcaaggtctccaacaaagccctc
ccagcccccatcgagaaaaccatctccaaagccaaagggcagccccgaga
accacaggtgtacaccaagcccccatcccgggaggagatgaccaagaacc
aggtcagcctgagctgcctggtcaaaggcttctatcccagcgacatcgcc
gtggagtgggagagcaatgggcagccggagaacaactacaagaccacggt
gcccgtgctggactccgacggctccttcagactcgcaagctatctcaccg
tggacaagagcagatggcagcaggggaacgtcttctcatgctccgtgatg
catgaggctctgcacaaccactacacgcagaagagcctctccctgtctcc
gggtaaa
human IgG1 Mono-Fc AA sequence
SEQ ID NO: 146
SPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKAL
PAPIEKTISKAKGQPREPQVYTKPPSREEMTKNQVSLSCLVKGFYPSDIA
VEWESNGQPENNYKTTVPVLDSDGSFRLASYLTVDKSRWQQGNVFSCSVM
HEALHNHYTQKSLSLSPGK
DNA sequence coding for VHH1
SEQ ID NO: 133
Caggtgcagctggtggagtctgggggaggattggtgcaggctgggggctc
tctgaaactcgcctgtgcagcacctggacttaccttcagttcgtatcgca
tgggctggttccgccaggctccagggcaggagcgtgagtttgtagcagct
attgattggaatggtcgtggcacatattatcgatactatgcagactccgt
gaagggccgatccaccatttccagagacaacgccaagaacacgatgtatc
tgcaaatgaacagcctgaaacctgaggacacggccgtttattactgtgca
gcaggttcgatcgaccttaactggtacggcggcatggactactggggcna
ngggacccaggtcaccgtctcctca
DNA sequence coding for VHH2
SEQ ID NO: 134
Gaggtgcaggtggtggagtctgggggaggattggtgcaggctgggggctc
tctgaaactcgcctgtgcagcacctggacttaccttcagttcgtatcgca
tgggctggttccgccaggctccagggcaggagcgtgagtttgtagcagct
attgattggaatggtcgtggcacatattatcgatactatgcagactccgt
gaagggccgatccaccatttccagagacaacgccaagaacacggtgtatc
tgcaaatgaacagcctgaaacctgaggacacggccgtttattactgtgca
gctactacggtattaactgaccctagggttcttaatgagtatgccacatg
gggccaggggacccaggtcaccgtctcctca
DNA sequence coding for VHH3
SEQ ID NO: 135
Cagttgcagctcgtggagtctgggggaggcttggtgcagcctggggggtc
tctgagactctcctgtgcagcctctggaagcatcttcagtatcaatgtta
tgggctggtaccgccaggctccagggaagcagcgcgagttggtcgcacgt
attaatggaggtggcattacacactatgcagagtccgtgaagggccgatt
caccatctccagagacaacgccaagaacacggtgtatctgcaaatgaaca
gcctgaaacctgaggacacagccgcatattactgtaaggcagatgtgttc
ggtagtagcgggtacgtagaaacctactggggccaggggacccaggtcac
cgtctcctca
DNA sequence coding for VHH4
SEQ ID NO: 136
Gaggtgcaggtggtggagtctgggggaggcttggtgcaggctgggggctc
tctgagactctcctgtgcagtctctggaacctccgtcagtagcaatgcca
tgggttggtaccgccaggctccagggaagcagcgcgagtgggtcggattt
attgatcgtattgctaccacgacgattgcaacctccgtgaagggccgatt
cgccatcaccagagacaacgccaagaacacggtgtatctccaaatgagcg
gcctgaaacctgaggacacagccgtctattactgtaatcatccattgacc
gctcggtggggccaggggacccaggtcaccgtctcctca
DNA sequence coding for VHH5
SEQ ID NO: 137
Caggtgcagctggtggagtctgggggaggcttggtgcaggctgggggctc
tctgagactctcctgtgcagcctctggacgcaccttcagtagctatgcca
tgggctggttccgccaggctccagggaaggagcgtgagtttgtagcagct
attacctggaatggtggtaccacatactatgcagactccgtgaagggccg
attcaccatctccagagacaacgccaagaacacggtgtatctgcaaatga
acagcctgaaacctgaggacacggccgtttattactgtgcagcagaccca
ttcaaccaaggctactggggccaggggacccaggtcaccgtctcctca
DNA sequence coding for VHH6
SEQ ID NO: 138
Gaggtgcagctcgtggagtctggaggaggcttggtgcaggctggggggtc
tctgagactctcctgtgcagtctctggaagctccgtcagtagcgatgcca
tgggttggtaccgccaggctccagggaatcagcgcgcgtgggtcgcattt
atttctggtggtggtaccacaacctatgcagactccgttaagggccgatt
caccatctccagagacaacaccaagaacacggtgtatctccacatgaaca
gcctgaaacctgaagacacagccgtctattactgtaatcatccattgacg
tctcggtggggccaggggacccaggtcaccgtctcctca
DNA sequence coding for VHH7
SEQ ID NO: 139
Gaggtgcaggtggtggagtctgggggaggattggtgcaggctggggggtc
tctgagactcgcctgtgtagcctctagaagcatcggcagtatcaatgtta
tgggctggtaccgccaggctccagggaagcagcgcgacttggtcgcacgt
attactggaggtggcagtacacactacgcagagtccgtgaagggccgatt
caccatctccagagacaacgccaagaacacggtgtatctgcaaatgaaca
gcctggaacctgaggacacggccgtttattactgtgcgtcaatggtaaac
cctatcattacggcttggggtacgattggtgtgcgcgagattcccgacta
tgactactggggccaggggacccaggtcaccgtctcctca
DNA sequence coding for VHH10
SEQ ID NO: 140
Gaggtgcaggtggtggagtctgggggaggcttggtgcaggctggggggtc
tctgagactctcctgtgcagcctctggattcaccttcacccgctatgcca
tgggctggttccgccaggctccagggaaggagcgatcgtttgtagcagct
attagctggagtggtagtagcgcaggctatggagactccgtgaagggccg
attcaccatctccagagacaacgccaagaacacgctgtatctgcaaatga
acagtctaaaacctgaggacacggccgtttattactgtgcagcagaccca
ttcaaccaaggctactggggccaggggacccaggtcaccgtctcctca
DNA sequence coding for VHH11
SEQ ID NO: 141
Gaggtgcaggtggtggagtctgggggaggattggtgcaggctgggggctc
tctgagactctcctgtgcagcctctggacgcaccttcactacctatcgca
tgggctggttccgccaggctccagggaaggagcgagagtttgtagcagct
attcgctggagtggtggtcgcacattgtatgcagactccgtgaagggccg
attcaccatctccagagacaacgccaagaacacagcgtatctgcaaatga
acaacctgagacctgaggacacggccgtttattactgtgcagcagatcta
gccgagtatagtggtacttactccagccctgcggactcccccgctgggta
tgactactggggccaggggacccaggtcaccgtctcctca
DNA sequence coding for VHH12
SEQ ID NO: 142
Caggtgcagctggtcgaaactgggggaggattggtgcaggctggggactc
tctgagactctcctgtgcagcctctggacgcaccctcagcttcaacacct
atgccatgggctggttccgccaggctccagggaaggagcgtgaatttgta
gcctctattacctggaatggtggaagcacaagctacgcagactccgtgaa
gggccgattcaccatcaccagagacaacgccaagaacacggctactctgc
gaatgaatagcctgcagcccgacgacacggccgtgtattactgtgcagca
gcccgatactatgtgagtggtacttacttccccgcgaattactggggcca
ggggacccaggtcaccgtctcctca
Exemplary stalk sequence of human pIgR
SEQ ID NO: 143
EKAVADTRDQADGSRASVDSGSSEEQGGSSR
Exemplary stalk sequence of mouse pIgR
SEQ ID NO: 144
EREIQNVGDQAQENRASGDAGSADGQSRSSSSK
Exemplary stalk sequence of mouse pIgR
SEQ ID NO: 145
EREIQNVRDQAQENRASGDAGSADGQSRSSSSK
Exemplary flexible linker 1
SEQ ID NO: 147
(EAAAK)n, wherein n is an integer from 1 to 20
Exemplary flexible linker 2
SEQ ID NO: 148
(GGGGS)n, wherein n is an integer from 1 to 20
Exemplary flexible linker 3
SEQ ID NO: 149
(GGGS)n, wherein n is an integer from 1 to 20
Exemplary hinge region 1
SEQ ID NO: 150
EPKSCDKTHTCPPCP
Exemplary hinge region 2
SEQ ID NO: 151
ERKCCVECPPCP
Exemplary hinge region 3
SEQ ID NO: 152
ELKTPLGDTTHTCPRCP(EPKSCDTPPPCPRCP)₃
Exemplary hinge region 4
SEQ ID NO: 153
ESKYGPPCPSCP
VHH1 and VHH2 CDR1 (Exemplary)
SEQ ID NO: 154
GLTFSSYRMG
VHH3 CDR1 (Exemplary)
SEQ ID NO: 155
GSIFSINVMG
VHH4 CDR1 (Exemplary)
SEQ ID NO: 156
GTSVSSNAMG
VHH5 CDR1 (Exemplary)
SEQ ID NO: 157
GRTFSSYAMG
VHH6 CDR1 (Exemplary)
SEQ ID NO: 158
GSSVSSDAMG
VHH7 CDR1 (Exemplary)
SEQ ID NO: 159
RSIGSINVMG
VHH9 CDR1 (Exemplary)
SEQ ID NO: 160
GRTFSTYRMG
VHH10 CDR1 (Exemplary)
SEQ ID NO: 161
GFTFTRYAMG
VHH11 CDR1 (Exemplary)
SEQ ID NO: 162
GRTFTTYRMG
VHH12 CDR1 (Exemplary)
SEQ ID NO: 163
GRTLSFNTYAMG
VHH1 and VHH2 CDR1 (Contact)
SEQ ID NO: 164
SSYRMG
VHH3 CDR1 (Contact)
SEQ ID NO: 165
SINVMG
VHH4 CDR1 (Contact)
SEQ ID NO: 166
SSNAMG
VHH5 CDR1 (Contact)
SEQ ID NO: 167
SSYAMG
VHH6 CDR1 (Contact)
SEQ ID NO: 168
SSDAMG
VHH7 CDR1 (Contact)
SEQ ID NO: 169
SINVMG
VHH9 CDR1 (Contact)
SEQ ID NO: 170
STYRMG
VHH10 CDR1 (Contact)
SEQ ID NO: 171
TRYAMG
VHH11 CDR1 (Contact)
SEQ ID NO: 172
TTYRMG
VHH12 CDR1 (Contact)
SEQ ID NO: 173
SFNTYAMG
VHH1 and VHH2 CDR1 (AbM)
SEQ ID NO: 174
GLTFSSYRMG
VHH3 CDR1 (AbM)
SEQ ID NO: 175
GSIFSINVMG
VHH4 CDR1 (AbM)
SEQ ID NO: 176
GTSVSSNAMG
VHH5 CDR1 (AbM)
SEQ ID NO: 177
GRTFSSYAMG
VHH6 CDR1 (AbM)
SEQ ID NO: 178
GSSVSSDAMG
VHH7 CDR1 (AbM)
SEQ ID NO: 179
RSIGSINVMG
VHH9 CDR1 (AbM)
SEQ ID NO: 180
GRTFSTYRMG
VHH10 CDR1 (AbM)
SEQ ID NO: 181
GFTFTRYAMG
VHH11 CDR1 (AbM)
SEQ ID NO: 182
GRTFTTYRMG
VHH12 CDR1 (AbM)
SEQ ID NO: 183
GRTLSFNTYAMG
VHH1 and VHH2 CDR2 (Exemplary)
SEQ ID NO: 184
AIDWNGRGTYYRYYADSVKG
VHH3 CDR2 (Exemplary)
SEQ ID NO: 185
RINGGGITHYAESVKG
VHH4 CDR2 (Exemplary)
SEQ ID NO: 186
FIDRIATTTIATSVKG
VHH5 CDR2 (Exemplary)
SEQ ID NO: 187
AITWNGGTTYYADSVKG
VHH6 CDR2 (Exemplary)
SEQ ID NO: 188
FISGGGTTTYADSVKG
VHH7 CDR2 (Exemplary)
SEQ ID NO: 189
RITGGGSTHYAESVKG
VHH9 CDR2 (Exemplary)
SEQ ID NO: 190
AISWSGGSTTYADPVKG
VHH10 CDR2 (Exemplary)
SEQ ID NO: 191
AISWSGSSAGYGDSVKG
VHH11 CDR2 (Exemplary)
SEQ ID NO: 192
AIRWSGGRTLYADSVKG
VHH12 CDR2 (Exemplary)
SEQ ID NO: 193
SITWNGGSTSYADSVKG
VHH1 and VHH2 CDR2 (Contact)
SEQ ID NO: 194
FVAAIDWNGRGTYYRY
VHH3 CDR2 (Contact)
SEQ ID NO: 195
LVARINGGGITH
VHH4 CDR2 (Contact)
SEQ ID NO: 196
WVGFIDRIATTT
VHH5 CDR2 (Contact)
SEQ ID NO: 197
FVAAITWNGGTTY
VHH6 CDR2 (Contact)
SEQ ID NO: 198
WVAFISGGGTTT
VHH7 CDR2 (Contact)
SEQ ID NO: 199
LVARITGGGSTH
VHH9 CDR2 (Contact)
SEQ ID NO: 200
FVAAISWSGGSTT
VHH10 CDR2 (Contact)
SEQ ID NO: 201
FVAAISWSGSSAG
VHH11 CDR2 (Contact)
SEQ ID NO: 202
FVAAIRWSGGRTL
VHH12 CDR2 (Contact)
SEQ ID NO: 203
FVASITWNGGSTS
VHH1 and VHH2 CDR2 (AbM)
SEQ ID NO: 204
AIDWNGRGTYYRY
VHH3 CDR2 (AbM)
SEQ ID NO: 205
RINGGGITH
VHH4 CDR2 (AbM)
SEQ ID NO: 206
FIDRIATTT
VHH5 CDR2 (AbM)
SEQ ID NO: 207
AITWNGGTTY
VHH6 CDR2 (AbM)
SEQ ID NO: 208
FISGGGTTT
VHH7 CDR2 (AbM)
SEQ ID NO: 209
RITGGGSTH
VHH9 CDR2 (AbM)
SEQ ID NO: 210
AISWSGGSTT
VHH10 CDR2 (AbM)
SEQ ID NO: 211
AISWSGSSAG
VHH11 CDR2 (AbM)
SEQ ID NO: 212
AIRWSGGRTL
VHH12 CDR2 (AbM)
SEQ ID NO: 213
SITWNGGSTS
VHH1 CDR3 (Exemplary)
SEQ ID NO: 214
GSIDLNWYGGMDY
VHH2 CDR3 (Exemplary)
SEQ ID NO: 215
TTVLTDPRVLNEYAT
VHH3 CDR3 (Exemplary)
SEQ ID NO: 216
DVFGSSGYVETY
VHH4 CDR3 (Exemplary)
SEQ ID NO: 217
PLTAR
VHH5 CDR3 (Exemplary)
SEQ ID NO: 218
DPFNQGY
VHH6 CDR3 (Exemplary)
SEQ ID NO: 219
PLTSR
VHH7 CDR3 (Exemplary)
SEQ ID NO: 220
MVNPIITAWGTIGVREIPDYDY
VHH9 CDR3 (Exemplary)
SEQ ID NO: 221
QRGY
VHH10 CDR3 (Exemplary)
SEQ ID NO: 222
DPFNQGY
VHH11 CDR3 (Exemplary)
SEQ ID NO: 223
DLAEYSGTYSSPADSPAGYDY
VHH12 CDR3 (Exemplary)
SEQ ID NO: 224
ARYYVSGTYFPANY
VHH1 CDR3 (Contact)
SEQ ID NO: 225
AAGSIDLNWYGGMD
VHH2 CDR3 (Contact)
SEQ ID NO: 226
AATTVLTDPRVLNEYA
VHH3 CDR3 (Contact)
SEQ ID NO: 227
KADVFGSSGYVET
VHH4 CDR3 (Contact)
SEQ ID NO: 228
NHPLTA
VHH5 CDR3 (Contact)
SEQ ID NO: 229
AADPFNQG
VHH6 CDR3 (Contact)
SEQ ID NO: 230
NHPLTS
VHH7 CDR3 (Contact)
SEQ ID NO: 231
ASMVNPIITAWGTIGVREIPDYD
VHH9 CDR3 (Contact)
SEQ ID NO: 232
NDQRG
VHH10 CDR3 (Contact)
SEQ ID NO: 233
AADPFNQG
VHH11 CDR3 (Contact)
SEQ ID NO: 234
AADLAEYSGTYSSPADSPAGYD
VHH12 CDR3 (Contact)
SEQ ID NO: 235
AAARYYVSGTYFPAN
VHH1 CDR3 (AbM)
SEQ ID NO: 236
GSIDLNWYGGMDY
VHH2 CDR3 (AbM)
SEQ ID NO: 237
TTVLTDPRVLNEYAT
VHH3 CDR3 (AbM)
SEQ ID NO: 238
DVFGSSGYVETY
VHH4 CDR3 (AbM)
SEQ ID NO: 239
PLTAR
VHH5 CDR3 (AbM)
SEQ ID NO: 240
DPFNQGY
VHH6 CDR3 (AbM)
SEQ ID NO: 241
PLTSR
VHH7 CDR3 (AbM)
SEQ ID NO: 242
MVNPIITAWGTIGVREIPDYDY
VHH9 CDR3 (AbM)
SEQ ID NO: 243
QRGY
VHH10 CDR3 (AbM)
SEQ ID NO: 244
DPFNQGY
VHH11 CDR3 (AbM)
SEQ ID NO: 245
DLAEYSGTYSSPADSPAGYDY
VHH12 CDR3 (AbM)
SEQ ID NO: 246
ARYYVSGTYFPANY
hpIgR 073
SEQ ID NO: 247
EVQVVESGGGLVQAGGSLRLSCAVSGTSVSSNAMGWYRQAPGKQREWVGF
IDRIATTTIATSVKGRFAITRDNAKNTVYLQMSGLKPEDTAVYYCXHPXT
ARWGQGTQVTVSS
hpIgR 201
SEQ ID NO: 248
QVQLVESGGGLVQPGGSLRLSCAASGRPNSKYAMAWFRRAPGKEREFQAA
INWSGANTYYGDSVKGRFTISRDNAKNTVTLQMNNLNPEDTAVYYCAADN
RAYTYHTFDYYQTDASYVYWGQGTQVTVSS
mpIgR 349
SEQ ID NO: 249
QLQLVESGGGLVQAGGSLRLSCAASGRTFSTSTMGWFRQAPGKEREFVAA
IQWSSASASTYYYYADSVKGRFTISRDNARNTVSLQMNSLKPEDTAVYYC
ANLVFRVGALKERDDYWGQGTQVTVSS
hpIgR D1
SEQ ID NO: 250
KSPIFGPEEVNSVEGNSVSITCYYPPTSVNRHTRKYWCRQGARGGCITLI
SSEGYVSSKYAGRANLTNFPENGTFVVNIAQLSQDDSGRYKCGLGINSRG
LSFDVSLEVS
mpIgR D1
SEQ ID NO: 251
KSPIFGPQEVSSIEGDSVSITCYYPDTSVNRHTRKYWCRQGASGMCTTLI
SSNGYLSKEYSGRANLINFPENNTFVINIEQLTQDDTGSYKCGLGTSNRG
LSFDVSLEVS
Consensus
SEQ ID NO: 252
KSPIFGPX₁EVX₂SIEGX₃SVSITCYYPX₄TSVNRHTRKYWCRQGAX₅G
X₆CX₇TLISSX₈GYLSX₉X₁₀YAGRANLX₁₁NFPENX₁₂TFVINIX₁₃Q
LSQDDSGX₁₄YKCGLGX₁₅X₁₆X₁₇RGLSFDVSLEVS
X₁ is E or Q, X₂ is N or S, X₃ is N or D, X₄ is P
or D, X₅ is R or S, X₆ is G or M, X₇ is I or T,
X₈ is E or N, X₉ is S or K, X₁₀ is K or E, X₁₁ is
T or I, X₁₂ is G or N, X₁₃ is A or E, X₁₄ is R or
S, X₁₅ is I or T, X₁₆ is N or S, X₁₇ is S or N
tpIgR D1
SEQ ID NO: 253
RVTTVGDLAVLEGRSVMIPCHYGPQYASYVKYWCRGSVKDLCTSLVRSDA
PRGPAAAGEDKVVMFDDPVQQVFTVTMTELQKEDSGWYWCGVEVGGVWSA
DVTASLHINVIQG
hpIgR D1
SEQ ID NO: 254
KSPIFGPEEVNSVEGNSVSITCYYPPTSVNRHTRKYWCRQGARGGCITLI
SSEGYVSSKYAGRANLTNFPENGTFVVNIAQLSQDDSGRYKCGLGINSRG
LSFDVSLEV
hD1 tCDR1
SEQ ID NO: 255
KSPIFGPEEVNSVEGNSVSITCYYGPQYASYRKYWCRQGARGGCITLISS
EGYVSSKYAGRANLTNFPENGTFVVNIAQLSQDDSGRYKCGLGINSRGLS
FDVSLEV
hD1 tCDR2
SEQ ID NO: 256
KSPIFGPEEVNSVEGNSVSITCYYPPTSVNRHTRKYWCRQGARGGCITLI
SSDAPVSSKYAGRANLTNFPENGTFVVNIAQLSQDDSGRYKCGLGINSRG
LSFDVSLEV
hD1 tCDR3
SEQ ID NO: 257
KSPIFGPEEVNSVEGNSVSITCYYPPTSVNRHTRKYWCRQGARGGCITLI
SSEGYVSSKYAGRANLTNFPENGTFVVNIAQLSQDDSGRYKCGLGVGGVW
SADLSFDVSLEV
VHH9 CDR1 (Kabat)
SEQ ID NO: 258
FTTYRMG
VHH10 CDR1 (Kabat) - alternative
SEQ ID NO: 259
TYRMG
VHH1 and VHH2 CDR2 (Chothia) - alternative
SEQ ID NO: 260
WNGRGTY
VHH3 CDR2 (Chothia) - alternative
SEQ ID NO: 261
GGG
VHH4 CDR2 (Chothia) - alternative
SEQ ID NO: 262
RIA
VHH5 CDR2 (Chothia) - alternative
SEQ ID NO: 263
WNGG
VHH6 CDR2 (Chothia) - alternative
SEQ ID NO: 264
GGG
VHH7 CDR2 (Chothia) - alternative
SEQ ID NO: 265
GGG
VHH9 CDR2 (Chothia) - alternative
SEQ ID NO: 266
WSGG
VHH10 CDR2 (Chothia) - alternative
SEQ ID NO: 267
WSGS
VHH11 CDR2 (Chothia) - alternative
SEQ ID NO: 268
WSGG
VHH12 CDR2 (Chothia) - alternative
SEQ ID NO: 269
WNGG
VHH1 and VHH2 CDR2 (IMGT) - alternative
SEQ ID NO: 270
IDWNGRGTYYR
VHH9 CDR3 (Kabat) - alternative
SEQ ID NO: 271
QRGY
VHH1 CDR3 (Chothia) - alternative
SEQ ID NO: 272
SIDLNWYGGMD
VHH2 CDR3 (Chothia) - alternative
SEQ ID NO: 273
TVLTDPRVLNEYA
VHH3 CDR3 (Chothia) - alternative
SEQ ID NO: 274
VFGSSGYVET
VHH4 CDR3 (Chothia) - alternative
SEQ ID NO: 275
LTA
VHH5 CDR3 (Chothia) - alternative
SEQ ID NO: 276
PFNQG
VHH6 CDR3 (Chothia) - alternative
SEQ ID NO: 277
LTS
VHH7 CDR3 (Chothia) - alternative
SEQ ID NO: 278
VNPIITAWGTIGVREIPDYD
VHH9 CDR3 (Chothia) - alternative
SEQ ID NO: 279
RG
VHH10 CDR3 (Chothia) - alternative
SEQ ID NO: 280
PFNQG
VHH11 CDR3 (Chothia) - alternative
SEQ ID NO: 281
LAEYSGTYSSPADSPAGYD
VHH12 CDR3 (Chothia) - alternative
SEQ ID NO: 282
RYYVSGTYFPAN
VHH1 CDR3 (IMGT) - alternative
SEQ ID NO: 283
AAGSIDLNWYGGMDY
VHH2 CDR3 (IMGT) - alternative
SEQ ID NO: 284
AATTVLTDPRVLNEYAT
Germline Sequence 1
SEQ ID NO: 285
QVQLVESGGGLVQAGGSLKLACAAPGLTFSSYRMGWFRQAPGQEREFVAA
IDWNGRGTYYRYYADSVKGRSTISRDNAKNTMYLQMNSLKPEDTAVYYCA
Germline Sequence 2
SEQ ID NO: 286
EVQVVESGGGLVQAGGSLKLACAAPGLTFSSYRMGWFRQAPGQEREFVAA
IDWNGRGTYYRYYADSVKGRSTISRDNAKNTVYLQMNSLKPEDTAVYYCA
Germline Sequence 3
SEQ ID NO: 287
QLQLVESGGGLVQPGGSLRLSCAASGSIFSINVMGWYRQAPGKQRELVAR
INGGGITHYAESVKGRFTISRDNAKNTVYLQMNSLKPEDTAAYYCKA
Germline Sequence 4
SEQ ID NO: 288
EVQVVESGGGLVQAGGSLRLSCAVSGTSVSSNAMGWYRQAPGKQREWVGF
IDRIATTTIATSVKGRFAITRDNAKNTVYLQMSGLKPEDTAVYYCN
Germline Sequence 5
SEQ ID NO: 289
QVQLVESGGGLVQAGGSLRLSCAASGRTFSSYAMGWFRQAPGKEREFVAA
ITWNGGTTYYADSVKGRFTISRDNAKNTVYLQMNSLKPEDTAVYYCAA
Germline Sequence 6
SEQ ID NO: 290
EVQLVESGGGLVQAGGSLRLSCAVSGSSVSSDAMGWYRQAPGNQRAWVAF
ISGGGTTTYADSVKGRFTISRDNTKNTVYLHMNSLKPEDTAVYYCN
Germline Sequence 7
SEQ ID NO: 291
EVQVVESGGGLVQAGGSLRLACVASRSIGSINVMGWYRQAPGKQRDLVAR
ITGGGSTHYAESVKGRFTISRDNAKNTVYLQMNSLEPEDTAVYYC
Germline Sequence 8
SEQ ID NO: 292
QVQLVESGGGLVQAGGSLRLSCAVSGRTFSTYRMGWFRQAPGKERSFVAA
ISWSGGSTTYADPVKGRFTISRDNAKNTVYLRMNSLKPEDTAVYYCN
Germline Sequence 9
SEQ ID NO: 293
EVQVVESGGGLVQAGGSLRLSCAASGFTFTRYAMGWFRQAPGKERSFVAA
ISWSGSSAGYGDSVKGRFTISRDNAKNTLYLQMNSLKPEDTAVYYCA
Germline Sequence 10
SEQ ID NO: 294
EVQVVESGGGLVQAGGSLRLSCAASGRTFTTYRMGWFRQAPGKEREFVAA
IRWSGGRTLYADSVKGRFTISRDNAKNTAYLQMNNLRPEDTAVYYCAA
Germline Sequence 11
SEQ ID NO: 295
QVQLVETGGGLVQAGDSLRLSCAASGRTLSFNTYAMGWFRQAPGKEREFV
ASITWNGGSTSYADSVKGRFTITRDNAKNTATLRMNSLQPDDTAVYYCAA
J Segment 1
SEQ ID NO: 296
DYWGQGTQVTVSS
J Segment 2
SEQ ID NO: 297
EYATWGQGTQVTVSS
J Segment 3
SEQ ID NO: 298
YWGQGTQVTVSS
J Segment 4
SEQ ID NO: 299
WGQGTQVTVSS
J Segment 5
SEQ ID NO: 300
DYDYWGQGTQVTVSS
J Segment 6
SEQ ID NO: 301
WGQGTLVTVSS
J Segment 7
SEQ ID NO: 302
YDYWGQGTQVTVSS
J Segment 8
SEQ ID NO: 303
NYWGQGTQVTVSS

What is claimed:

1. A VHH domain that binds to an extracellular domain of polymeric immunoglobulin receptor (pIgR).

2. The VHH domain of claim 1, wherein the VHH domain binds to an extracellular domain 1 of pIgR.

3. The VHH domain of claim 1, wherein the VHH domain binds to an extracellular domain 2 of pIgR.

4. The VHH domain of claim 1, wherein the VHH domain binds to an extracellular domain 1-2 of pIgR.

5. The VHH domain of claim 1, wherein the VHH domain binds to an extracellular domain 3 of pIgR.

6. The VHH domain of claim 1, wherein the VHH domain binds to an extracellular domain 2-3 of pIgR.

7. The VHH domain of claim 1, wherein the VHH domain binds to an extracellular domain 4-5 of pIgR.

8. The VHH domain of claim 1, wherein the VHH domain binds to an extracellular domain 5 of pIgR.

9. The VHH domain of any one of claims 1 to 8, wherein pIgR is human pIgR.

10. The VHH domain of any one of claims 1 to 8, wherein pIgR is mouse pIgR.

11. The VHH domain of any one of claims 1 to 8, wherein the VHH domain does not detectably bind to the amino acid sequence of EKAVADTRDQADGSRASVDSGSSEEQGGSSR (SEQ ID NO: 143), EREIQNVGDQAQENRASGDAGSADGQSRSSSSK (SEQ ID NO: 144), or EREIQNVRDQAQENRASGDAGSADGQSRSSSSK (SEQ ID NO: 145).

12. The VHH domain of claim 1, wherein the VHH domain competes with IgA binding to the pIgR.

13. The VHH domain of claim 1, wherein the VHH domain promotes IgA binding to the pIgR.

14. The VHH domain of any one of claims 1 to 13, wherein the K_{D} of the binding of the VHH domain to pIgR is from about 4 to about 525 nM.

15. The VHH domain of any one of claims 1 to 14, wherein the K_{D} of the binding of the VHH domain to pIgR is less than about 50 nM.

16. The VHH domain of any one of claims 1 to 15, wherein the K_{D} of the binding of the VHH domain to pIgR is from about 4 to about 34 nM.

17. The VHH domain of any one of claims 1 to 16, wherein the Tₘ of the VHH domain is from about 53 to about 77° C.

18. The VHH domain of any one of claims 1 to 17, wherein the Tₘ of the VHH domain is from 53.9 to 76.4° C.

19. The VHH domain of any one of claims 1 to 18, wherein the VHH domain comprises a CDR3 sequence of GSIDLNWYGGMDY (SEQ ID NO: 60), TTVLTDPRVLNEYAT (SEQ ID NO: 61), DVFGSSGYVETY (SEQ ID NO: 62), PLTAR (SEQ ID NO: 63), DPFNQGY (SEQ ID NO: 64), PLTSR (SEQ ID NO: 65), MVNPIITAWGTIGVREIPDYDY (SEQ ID NO: 66), DQRGY (SEQ ID NO: 67), QRGY (SEQ ID NO: 271), DPFNQGY (SEQ ID NO: 68), DLAEYSGTYSSPADSPAGYDY (SEQ ID NO: 69), ARYYVSGTYFPANY (SEQ ID NO: 70), GSIDLNWYGGMDY (SEQ ID NO: 71), SIDLNWYGGMD (SEQ ID NO: 272), TTVLTDPRVLNEYAT (SEQ ID NO: 72), TVLTDPRVLNEYA (SEQ ID NO: 273), DVFGSSGYVETY (SEQ ID NO: 73), VFGSSGYVET (SEQ ID NO: 274), PLTAR (SEQ ID NO: 74), LTA (SEQ ID NO: 275), DPFNQGY (SEQ ID NO: 75), PFNQG (SEQ ID NO: 276), PLTSR (SEQ ID NO: 76), LTS (SEQ ID NO: 277), MVNPIITAWGTIGVREIPDYDY (SEQ ID NO: 77), VNPIITAWGTIGVREIPDYD (SEQ ID NO: 278), DQRGY (SEQ ID NO: 78), RG (SEQ ID NO: 279), DPFNQGY (SEQ ID NO: 79), PFNQG (SEQ ID NO: 280), DLAEYSGTYSSPADSPAGYDY (SEQ ID NO: 80), LAEYSGTYSSPADSPAGYD (SEQ ID NO: 281), ARYYVSGTYFPANY (SEQ ID NO: 81), RYYVSGTYFPAN (SEQ ID NO: 282), CAAGSIDLNWYGGMDY (SEQ ID NO: 82), AAGSIDLNWYGGMDY (SEQ ID NO: 283), CAATTVLTDPRVLNEYAT (SEQ ID NO: 83), AATTVLTDPRVLNEYAT (SEQ ID NO: 284), KADVFGSSGYVETY (SEQ ID NO: 84), NHPLTAR (SEQ ID NO: 85), AADPFNQGY (SEQ ID NO: 86), NHPLTSR (SEQ ID NO: 87), ASMVNPIITAWGTIGVREIPDYDY (SEQ ID NO: 88), NDQRGY (SEQ ID NO: 89), AADPFNQGY (SEQ ID NO: 90), AADLAEYSGTYSSPADSPAGYDY (SEQ ID NO: 91), AAARYYVSGTYFPANY (SEQ ID NO: 92), GSIDLNWYGGMDY (SEQ ID NO: 214), TTVLTDPRVLNEYAT (SEQ ID NO: 215), DVFGSSGYVETY (SEQ ID NO: 216), PLTAR (SEQ ID NO: 217), DPFNQGY (SEQ ID NO: 218), PLTSR (SEQ ID NO: 219), MVNPIITAWGTIGVREIPDYDY (SEQ ID NO: 220), QRGY (SEQ ID NO: 221), DPFNQGY (SEQ ID NO: 222), DLAEYSGTYSSPADSPAGYDY (SEQ ID NO: 223), ARYYVSGTYFPANY (SEQ ID NO: 224), AAGSIDLNWYGGMD (SEQ ID NO: 225), AATTVLTDPRVLNEYA (SEQ ID NO: 226), KADVFGSSGYVET (SEQ ID NO: 227), NHPLTA (SEQ ID NO: 228), AADPFNQG (SEQ ID NO: 229), NHPLTS (SEQ ID NO: 230), ASMVNPIITAWGTIGVREIPDYD (SEQ ID NO: 231), NDQRG (SEQ ID NO: 232), AADPFNQG (SEQ ID NO: 233), AADLAEYSGTYSSPADSPAGYD (SEQ ID NO: 234), AAARYYVSGTYFPAN (SEQ ID NO: 235), GSIDLNWYGGMDY (SEQ ID NO: 236), TTVLTDPRVLNEYAT (SEQ ID NO: 237), DVFGSSGYVETY (SEQ ID NO: 238), PLTAR (SEQ ID NO: 239), DPFNQGY (SEQ ID NO: 240), PLTSR (SEQ ID NO: 241), MVNPIITAWGTIGVREIPDYDY (SEQ ID NO: 242), QRGY (SEQ ID NO: 243), DPFNQGY (SEQ ID NO: 244), DLAEYSGTYSSPADSPAGYDY (SEQ ID NO: 245), or ARYYVSGTYFPANY (SEQ ID NO: 246).

20. The VHH domain of any one of claims 1 to 9, wherein the VHH domain comprises a CDR2 sequence of AIDWNGRGTYYRYYADSVKG (SEQ ID NO: 30), RINGGGITHYAESVKG (SEQ ID NO: 31), FIDRIATTTIATSVKG (SEQ ID NO: 32), AITWNGGTTYYADSVKG (SEQ ID NO: 33), FISGGGTTTYADSVKG (SEQ ID NO: 34), RITGGGSTHYAESVKG (SEQ ID NO: 35), AISWSGGSTTYADPVKG (SEQ ID NO: 36), AISWSGSSAGYGDSVKG (SEQ ID NO: 37), AIRWSGGRTLYADSVKG (SEQ ID NO: 38), SITWNGGSTSYADSVKG (SEQ ID NO: 39), DWNGRGTYY (SEQ ID NO: 40), WNGRGTY (SEQ ID NO: 260), NGGGI (SEQ ID NO: 41), GGG (SEQ ID NO: 261), DRIAT (SEQ ID NO: 42), RIA (SEQ ID NO: 262), TWNGGT (SEQ ID NO: 43), WNGG (SEQ ID NO: 263), SGGGT (SEQ ID NO: 44), GGG (SEQ ID NO: 264), TGGGS (SEQ ID NO: 45), GGG (SEQ ID NO: 265), SWSGGS (SEQ ID NO: 46), WSGG (SEQ ID NO: 266), SWSGSS (SEQ ID NO: 47), WSGS (SEQ ID NO: 267), RWSGGR (SEQ ID NO: 48), WSGG (SEQ ID NO: 268), TWNGGS (SEQ ID NO: 49), WNGG (SEQ ID NO: 269), IDWNGRGTYY (SEQ ID NO: 50), IDWNGRGTYYR (SEQ ID NO: 270), INGGGIT (SEQ ID NO: 51), IDRIATT (SEQ ID NO: 52), ITWNGGTT (SEQ ID NO: 53), ISGGGTT (SEQ ID NO: 54), ITGGGST (SEQ ID NO: 55), ISWSGGST (SEQ ID NO: 56), ISWSGSSA (SEQ ID NO: 57), IRWSGGRT (SEQ ID NO: 58), ITWNGGST (SEQ ID NO: 59), AIDWNGRGTYYRYYADSVKG (SEQ ID NO: 184), RINGGGITHYAESVKG (SEQ ID NO: 185), FIDRIATTTIATSVKG (SEQ ID NO: 186), AITWNGGTTYYADSVKG (SEQ ID NO: 187), FISGGGTTTYADSVKG (SEQ ID NO: 188), RITGGGSTHYAESVKG (SEQ ID NO: 189), AISWSGGSTTYADPVKG (SEQ ID NO: 190), AISWSGSSAGYGDSVKG (SEQ ID NO: 191), AIRWSGGRTLYADSVKG (SEQ ID NO: 192), SITWNGGSTSYADSVKG (SEQ ID NO: 193), FVAAIDWNGRGTYYRY (SEQ ID NO: 194), LVARINGGGITH (SEQ ID NO: 195), WVGFIDRIATTT (SEQ ID NO: 196), FVAAITWNGGTTY (SEQ ID NO: 197), WVAFISGGGTTT (SEQ ID NO: 198), LVARITGGGSTH (SEQ ID NO: 199), FVAAISWSGGSTT (SEQ ID NO: 200), FVAAISWSGSSAG (SEQ ID NO: 201), FVAAIRWSGGRTL (SEQ ID NO: 202), FVASITWNGGSTS (SEQ ID NO: 203), AIDWNGRGTYYRY (SEQ ID NO: 204), RINGGGITH (SEQ ID NO: 205), FIDRIATTT (SEQ ID NO: 206), AITWNGGTTY (SEQ ID NO: 207), FISGGGTTT (SEQ ID NO: 208), RITGGGSTH (SEQ ID NO: 209), AISWSGGSTT (SEQ ID NO: 210), AISWSGSSAG (SEQ ID NO: 211), AIRWSGGRTL (SEQ ID NO: 212), or SITWNGGSTS (SEQ ID NO: 213).

21. The VHH domain of any one of claims 1 to 10, wherein the VHH domain comprises a CDR1 sequence of SYRMG (SEQ ID NO: 1), INVMG (SEQ ID NO: 2), SNAMG (SEQ ID NO: 3), SYAMG (SEQ ID NO: 4), SDAMG (SEQ ID NO: 5), INVMG (SEQ ID NO: 6), TYRMG (SEQ ID NO: 7), RYAMG (SEQ ID NO: 8), FTTYRMG (SEQ ID NO: 258), TYRMG (SEQ ID NO: 259), FNTYAMG (SEQ ID NO: 9), GLTFSSY (SEQ ID NO: 10), GSIFSIN (SEQ ID NO: 11), GTSVSSN (SEQ ID NO: 12), GRTFSSY (SEQ ID NO: 13), GSSVSSD (SEQ ID NO: 14), RSIGSIN (SEQ ID NO: 15), GRTFSTY (SEQ ID NO: 16), GFTFTRY (SEQ ID NO: 17), GRTFTTY (SEQ ID NO: 18), GRTLSFNTY (SEQ ID NO: 19), GLTFSSYR (SEQ ID NO: 20), GSIFSINV (SEQ ID NO: 21), GTSVSSNA (SEQ ID NO: 22), GRTFSSYA (SEQ ID NO: 23), GSSVSSDA (SEQ ID NO: 24), RSIGSINV (SEQ ID NO: 25), GRTFSTYR (SEQ ID NO: 26), GFTFTRYA (SEQ ID NO: 27), GRTFTTYR (SEQ ID NO: 28), GRTLSFNTYA (SEQ ID NO: 29), GLTFSSYRMG (SEQ ID NO: 154), GSIFSINVMG (SEQ ID NO: 155), GTSVSSNAMG (SEQ ID NO: 156), GRTFSSYAMG (SEQ ID NO: 157), GSSVSSDAMG (SEQ ID NO: 158), RSIGSINVMG (SEQ ID NO: 159), GRTFSTYRMG (SEQ ID NO: 160), GFTFTRYAMG (SEQ ID NO: 161), GRTFTTYRMG (SEQ ID NO: 162), GRTLSFNTYAMG (SEQ ID NO: 163), SSYRMG (SEQ ID NO: 164), SINVMG (SEQ ID NO: 165), SSNAMG (SEQ ID NO: 166), SSYAMG (SEQ ID NO: 167), SSDAMG (SEQ ID NO: 168), SINVMG (SEQ ID NO: 169), STYRMG (SEQ ID NO: 170), TRYAMG (SEQ ID NO: 171), TTYRMG (SEQ ID NO: 172), SFNTYAMG (SEQ ID NO: 173), GLTFSSYRMG (SEQ ID NO: 174), GSIFSINVMG (SEQ ID NO: 175), GTSVSSNAMG (SEQ ID NO: 176), GRTFSSYAMG (SEQ ID NO: 177), GSSVSSDAMG (SEQ ID NO: 178), RSIGSINVMG (SEQ ID NO: 179), GRTFSTYRMG (SEQ ID NO: 180), GFTFTRYAMG (SEQ ID NO: 181), GRTFTTYRMG (SEQ ID NO: 182), or GRTLSFNTYAMG (SEQ ID NO: 183).

22. The VHH domain of any one of claims 1 to 10, wherein the VHH domain comprises a CDR1 sequence, a CDR2 sequence, and a CDR3 sequence of the VHH domain selected from the group consisting of:
a) VHH1:
i) the CDR1 sequence of SYRMG (SEQ ID NO: 1), the CDR2 sequence of AIDWNGRGTYYRYYADSVKG (SEQ ID NO: 30), and the CDR3 sequence of GSIDLNWYGGMDY (SEQ ID NO: 60);
ii) the CDR1 sequence of GLTFSSY (SEQ ID NO: 10), the CDR2 sequence of DWNGRGTYY (SEQ ID NO: 40) or WNGRGTY (SEQ ID NO: 260), and the CDR3 sequence of GSIDLNWYGGMDY (SEQ ID NO: 71) or SIDLNWYGGMD (SEQ ID NO: 272);
iii) the CDR1 sequence of GLTFSSYR (SEQ ID NO: 20), the CDR2 sequence of IDWNGRGTYY (SEQ ID NO: 50) or IDWNGRGTYYR (SEQ ID NO: 270), and the CDR3 sequence of CAAGSIDLNWYGGMDY (SEQ ID NO: 82) or AAGSIDLNWYGGMDY (SEQ ID NO: 283);
iv) the CDR1 sequence of GLTFSSYRMG (SEQ ID NO: 154), the CDR 2 sequence of AIDWNGRGTYYRYYADSVKG (SEQ ID NO: 184), and the CDR3 sequence of GSIDLNWYGGMDY (SEQ ID NO: 214);
v) the CDR1 sequence of SSYRMG (SEQ ID NO: 164), the CDR2 sequence of FVAAIDWNGRGTYYRY (SEQ ID NO: 194), and the CDR3 sequence of AAGSIDLNWYGGMD (SEQ ID NO: 225); or
vi) the CDR1 sequence of GLTFSSYRMG (SEQ ID NO: 174), the CDR2 sequence of AIDWNGRGTYYRY (SEQ ID NO: 204), and the CDR3 sequence of GSIDLNWYGGMDY (SEQ ID NO: 236);
b) VHH2:
i) the CDR1 sequence of SYRMG (SEQ ID NO: 1), the CDR2 sequence of AIDWNGRGTYYRYYADSVKG (SEQ ID NO: 30), and the CDR3 sequence of TTVLTDPRVLNEYAT (SEQ ID NO: 61);
ii) the CDR1 sequence of GLTFSSY (SEQ ID NO: 10), the CDR2 sequence of DWNGRGTYY (SEQ ID NO: 40) or WNGRGTY (SEQ ID NO: 260), and the CDR3 sequence of TTVLTDPRVLNEYAT (SEQ ID NO: 72) or TVLTDPRVLNEYA (SEQ ID NO: 273);
iii) the CDR1 sequence of GLTFSSYR (SEQ ID NO: 20), the CDR2 sequence of IDWNGRGTYY (SEQ ID NO: 50) or IDWNGRGTYYR (SEQ ID NO: 270), and the CDR3 sequence of CAATTVLTDPRVLNEYAT (SEQ ID NO: 83) or AATTVLTDPRVLNEYAT (SEQ ID NO: 284);
iv) the CDR1 sequence of GLTFSSYRMG (SEQ ID NO: 154), the CDR2 sequence of AIDWNGRGTYYRYYADSVKG (SEQ ID NO: 184), and the CDR3 sequence of TTVLTDPRVLNEYAT (SEQ ID NO: 215);
v) the CDR1 sequence of SSYRMG (SEQ ID NO: 164), the CDR2 sequence of FVAAIDWNGRGTYYRY (SEQ ID NO: 194), and the CDR3 sequence of AATTVLTDPRVLNEYA (SEQ ID NO: 226); or
vi) the CDR1 sequence of GLTFSSYRMG (SEQ ID NO: 174), the CDR2 sequence of AIDWNGRGTYYRY (SEQ ID NO: 204), and the CDR3 sequence of TTVLTDPRVLNEYAT (SEQ ID NO: 237);
c) VHH3:
i) the CDR1 sequence of INVMG (SEQ ID NO: 2), the CDR2 sequence of RINGGGITHYAESVKG (SEQ ID NO: 31), and the CDR3 sequence of DVFGSSGYVETY (SEQ ID NO: 62);
ii) the CDR1 sequence of GSIFSIN (SEQ ID NO: 11), the CDR2 sequence of NGGGI (SEQ ID NO: 41) or GGG (SEQ ID NO: 261), and the CDR3 sequence of DVFGSSGYVETY (SEQ ID NO: 73) or VFGSSGYVET (SEQ ID NO: 274);
iii) the CDR1 sequence of GSIFSINV (SEQ ID NO: 21), the CDR2 sequence of INGGGIT (SEQ ID NO: 51), and the CDR3 sequence of KADVFGSSGYVETY (SEQ ID NO: 84);
iv) the CDR1 sequence of GSIFSINVMG (SEQ ID NO: 155), the CDR2 sequence of RINGGGITHYAESVKG (SEQ ID NO: 185), and the CDR3 sequence of DVFGSSGYVETY (SEQ ID NO: 216);
v) the CDR1 sequence of SINVMG (SEQ ID NO: 165), the CDR2 sequence of LVARINGGGITH (SEQ ID NO: 195), and the CDR3 sequence of KADVFGSSGYVET (SEQ ID NO: 227); or
vi) the CDR1 sequence of GSIFSINVMG (SEQ ID NO: 175), the CDR2 sequence of RINGGGITH (SEQ ID NO: 205), and the CDR3 sequence of DVFGSSGYVETY (SEQ ID NO: 238);
d) VHH4:
i) the CDR1 sequence of SNAMG (SEQ ID NO: 3), the CDR2 sequence of FIDRIATTTIATSVKG (SEQ ID NO: 32), and the CDR3 sequence of PLTAR (SEQ ID NO: 63);
ii) the CDR1 sequence of GTSVSSN (SEQ ID NO: 12), the CDR2 sequence of DRIAT (SEQ ID NO: 42) or RIA (SEQ ID NO: 262), and the CDR3 sequence of PLTAR (SEQ ID NO: 74) or LTA (SEQ ID NO: 275);
iii) the CDR1 sequence of GTSVSSNA (SEQ ID NO: 22), the CDR2 sequence of IDRIATT (SEQ ID NO: 52), and the CDR3 sequence of NHPLTAR (SEQ ID NO: 85);
iv) the CDR1 sequence of GTSVSSNAMG (SEQ ID NO: 156), the CDR2 sequence of FIDRIATTTIATSVKG (SEQ ID NO: 186), and the CDR3 sequence of PLTAR (SEQ ID NO: 217);
v) the CDR1 sequence of SSNAMG (SEQ ID NO: 166), the CDR2 sequence of WVGFIDRIATTT (SEQ ID NO: 196), and the CDR3 sequence of NHPLTA (SEQ ID NO: 228); or
vi) the CDR1 sequence of GTSVSSNAMG (SEQ ID NO: 176), the CDR2 sequence of FIDRIATTT (SEQ ID NO: 206), and the CDR3 sequence of PLTAR (SEQ ID NO: 239);
e) VHH5:
i) the CDR1 sequence of SYAMG (SEQ ID NO: 4), the CDR2 sequence of AITWNGGTTYYADSVKG (SEQ ID NO: 33), and the CDR3 sequence of DPFNQGY (SEQ ID NO: 64);
ii) the CDR1 sequence of GRTFSSY (SEQ ID NO: 13), the CDR2 sequence of TWNGGT (SEQ ID NO: 43) or WNGG (SEQ ID NO: 263), and the CDR3 sequence of DPFNQGY (SEQ ID NO: 75) or PFNQG (SEQ ID NO: 276);
iii) the CDR1 sequence of GRTFSSYA (SEQ ID NO: 23), the CDR2 sequence of ITWNGGTT (SEQ ID NO: 53), and the CDR3 sequence of AADPFNQGY (SEQ ID NO: 86);
iv) the CDR1 sequence of GRTFSSYAMG (SEQ ID NO: 157), the CDR2 sequence of AITWNGGTTYYADSVKG (SEQ ID NO: 187), and the CDR3 sequence of DPFNQGY (SEQ ID NO: 218);
v) the CDR1 sequence of SSYAMG (SEQ ID NO: 167), the CDR2 sequence of FVAAITWNGGTTY (SEQ ID NO: 197), and the CDR3 sequence of AADPFNQG (SEQ ID NO: 229); or
vi) the CDR1 sequence of GRTFSSYAMG (SEQ ID NO: 177), the CDR2 sequence of AITWNGGTTY (SEQ ID NO: 207), and the CDR3 sequence of DPFNQGY (SEQ ID NO: 240);
f) VHH6:
i) the CDR1 sequence of SDAMG (SEQ ID NO: 5), the CDR2 sequence of FISGGGTTTYADSVKG (SEQ ID NO: 34), and the CDR3 sequence of PLTSR (SEQ ID NO: 65);
ii) the CDR1 sequence of GSSVSSD (SEQ ID NO: 14), the CDR2 sequence of SGGGT (SEQ ID NO: 44) or GGG (SEQ ID NO: 264), and the CDR3 sequence of PLTSR (SEQ ID NO: 76) or LTS (SEQ ID NO: 277);
iii) the CDR1 sequence of GSSVSSDA (SEQ ID NO: 24), the CDR2 sequence of ISGGGTT (SEQ ID NO: 54), and the CDR3 sequence of NHPLTSR (SEQ ID NO: 87);
iv) the CDR1 sequence of GSSVSSDAMG (SEQ ID NO: 158), the CDR2 sequence of FISGGGTTTYADSVKG (SEQ ID NO: 188), and the CDR3 sequence of PLTSR (SEQ ID NO: 219);
v) the CDR1 sequence of SSDAMG (SEQ ID NO: 168), the CDR2 sequence of WVAFISGGGTTT (SEQ ID NO: 198), and the CDR3 sequence of NHPLTS (SEQ ID NO: 230); or
vi) the CDR1 sequence of GSSVSSDAMG (SEQ ID NO: 178), the CDR2 sequence of FISGGGTTT (SEQ ID NO: 208), and the CDR3 sequence of PLTSR (SEQ ID NO: 241);
g) VHH7:
i) the CDR1 sequence of INVMG (SEQ ID NO: 6), the CDR2 sequence of RITGGGSTHYAESVKG (SEQ ID NO: 35), and the CDR3 sequence of MVNPIITAWGTIGVREIPDYDY (SEQ ID NO: 66);
ii) the CDR1 sequence of RSIGSIN (SEQ ID NO: 15), the CDR2 sequence of TGGGS (SEQ ID NO: 45) or GGG (SEQ ID NO: 265), and the CDR3 sequence of MVNPIITAWGTIGVREIPDYDY (SEQ ID NO: 77) or VNPIITAWGTIGVREIPDYD (SEQ ID NO: 278);
iii) the CDR1 sequence of RSIGSINV (SEQ ID NO: 25), the CDR2 sequence of ITGGGST (SEQ ID NO: 55), and the CDR3 sequence of ASMVNPIITAWGTIGVREIPDYDY (SEQ ID NO: 88);
iv) the CDR1 sequence of RSIGSINVMG (SEQ ID NO: 159), the CDR2 sequence of RITGGGSTHYAESVKG (SEQ ID NO: 189), and the CDR3 sequence of MVNPIITAWGTIGVREIPDYDY (SEQ ID NO: 220);
v) the CDR1 sequence of SINVMG (SEQ ID NO: 169), the CDR2 sequence of LVARITGGGSTH (SEQ ID NO: 199), and the CDR3 sequence of ASMVNPIITAWGTIGVREIPDYD (SEQ ID NO: 231); or
vi) the CDR1 sequence of RSIGSINVMG (SEQ ID NO: 179), the CDR2 sequence of RITGGGSTH (SEQ ID NO: 209), and the CDR3 sequence of MVNPIITAWGTIGVREIPDYDY (SEQ ID NO: 242);
h) VHH9:
i) the CDR1 sequence of TYRMG (SEQ ID NO: 7), the CDR2 sequence of AISWSGGSTTYADPVKG (SEQ ID NO: 36), and the CDR3 sequence of DQRGY (SEQ ID NO: 67) or QRGY (SEQ ID NO: 271);
ii) the CDR1 sequence of GRTFSTY (SEQ ID NO: 16), the CDR2 sequence of SWSGGS (SEQ ID NO: 46) or WSGG (SEQ ID NO: 266), and the CDR3 sequence of DQRGY (SEQ ID NO: 78) or RG (SEQ ID NO: 279);
iii) the CDR1 sequence of GRTFSTYR (SEQ ID NO: 26), the CDR2 sequence of ISWSGGST (SEQ ID NO: 56), and the CDR3 sequence of NDQRGY (SEQ ID NO: 89);
iv) the CDR1 sequence of GRTFSTYRMG (SEQ ID NO: 160), the CDR2 sequence of AISWSGGSTTYADPVKG (SEQ ID NO: 190), and the CDR3 sequence of QRGY (SEQ ID NO: 221);
v) the CDR1 sequence of STYRMG (SEQ ID NO: 170), the CDR2 sequence of FVAAISWSGGSTT (SEQ ID NO: 200), and the CDR3 sequence of NDQRG (SEQ ID NO: 232); or
vi) the CDR1 sequence of GRTFSTYRMG (SEQ ID NO: 180), the CDR2 sequence of AISWSGGSTT (SEQ ID NO: 210), and the CDR3 sequence of QRGY (SEQ ID NO: 243);
i) VHH10:
i) the CDR1 sequence of RYAMG (SEQ ID NO: 8), the CDR2 sequence of AISWSGSSAGYGDSVKG (SEQ ID NO: 37), and the CDR3 sequence of DPFNQGY (SEQ ID NO: 68);
ii) the CDR1 sequence of GFTFTRY (SEQ ID NO: 17), the CDR2 sequence of SWSGSS (SEQ ID NO: 47) or WSGS (SEQ ID NO: 267), and the CDR3 sequence of DPFNQGY (SEQ ID NO: 79) or PFNQG (SEQ ID NO: 280);
iii) the CDR1 sequence of GFTFTRYA (SEQ ID NO: 27), the CDR2 sequence of ISWSGSSA (SEQ ID NO: 57), and the CDR3 sequence of AADPFNQGY (SEQ ID NO: 90);
iv) the CDR1 sequence of GFTFTRYAMG (SEQ ID NO: 161), the CDR2 sequence of AISWSGSSAGYGDSVKG (SEQ ID NO: 191), and the CDR3 sequence of DPFNQGY (SEQ ID NO: 222);
v) the CDR1 sequence of TRYAMG (SEQ ID NO: 171), the CDR2 sequence of FVAAISWSGSSAG (SEQ ID NO: 201), and the CDR3 sequence of AADPFNQG (SEQ ID NO: 233); or
vi) the CDR1 sequence of GFTFTRYAMG (SEQ ID NO: 181), the CDR2 sequence of AISWSGSSAG (SEQ ID NO: 211), and the CDR3 sequence of DPFNQGY (SEQ ID NO: 244);
j) VHH11:
i) the CDR1 sequence of FTTYRMG (SEQ ID NO: 258) or TYRMG (SEQ ID NO: 259), the CDR2 sequence of AIRWSGGRTLYADSVKG (SEQ ID NO: 38), and the CDR3 sequence of DLAEYSGTYSSPADSPAGYDY (SEQ ID NO: 69);
ii) the CDR1 sequence of GRTFTTY (SEQ ID NO: 18), the CDR2 sequence of RWSGGR (SEQ ID NO: 48) or WSGG (SEQ ID NO: 268), and the CDR3 sequence of DLAEYSGTYSSPADSPAGYDY (SEQ ID NO: 80) or LAEYSGTYSSPADSPAGYD (SEQ ID NO: 281);
iii) the CDR1 sequence of GRTFTTYR (SEQ ID NO: 28), the CDR2 sequence of IRWSGGRT (SEQ ID NO: 58), and the CDR3 sequence of AADLAEYSGTYSSPADSPAGYDY (SEQ ID NO: 91);
iv) the CDR1 sequence of GRTFTTYRMG (SEQ ID NO: 162), the CDR2 sequence of AIRWSGGRTLYADSVKG (SEQ ID NO: 192), and the CDR3 sequence of DLAEYSGTYSSPADSPAGYDY (SEQ ID NO: 223);
v) the CDR1 sequence of TTYRMG (SEQ ID NO: 172), the CDR2 sequence of FVAAIRWSGGRTL (SEQ ID NO: 202), and the CDR3 sequence of AADLAEYSGTYSSPADSPAGYD (SEQ ID NO: 234); or
vi) the CDR1 sequence of GRTFTTYRMG (SEQ ID NO: 182), the CDR2 sequence of AIRWSGGRTL (SEQ ID NO: 212), and the CDR3 sequence of DLAEYSGTYSSPADSPAGYDY (SEQ ID NO: 245); and
k) VHH12:
i) the CDR1 sequence of FNTYAMG (SEQ ID NO: 9), the CDR2 sequence of SITWNGGSTSYADSVKG (SEQ ID NO: 39), and the CDR3 sequence of ARYYVSGTYFPANY (SEQ ID NO: 70);
ii) the CDR1 sequence of GRTLSFNTY (SEQ ID NO: 19), the CDR2 sequence of TWNGGS (SEQ ID NO: 49) or WNGG (SEQ ID NO: 269), and the CDR3 sequence of ARYYVSGTYFPANY (SEQ ID NO: 81) or RYYVSGTYFPAN (SEQ ID NO: 282);
iii) the CDR1 sequence of GRTLSFNTYA (SEQ ID NO: 29), the CDR2 sequence of ITWNGGST (SEQ ID NO: 59), and the CDR3 sequence of AAARYYVSGTYFPANY (SEQ ID NO: 92);
iv) the CDR1 sequence of GRTLSFNTYAMG (SEQ ID NO: 163), the CDR2 sequence of SITWNGGSTSYADSVKG (SEQ ID NO: 193), and the CDR3 sequence of ARYYVSGTYFPANY (SEQ ID NO: 224);
v) the CDR1 sequence of SFNTYAMG (SEQ ID NO: 173), the CDR2 sequence of FVASITWNGGSTS (SEQ ID NO: 203), and the CDR3 sequence of AAARYYVSGTYFPAN (SEQ ID NO: 235); or
vi) the CDR1 sequence of GRTLSFNTYAMG (SEQ ID NO: 183), the CDR2 sequence of SITWNGGSTS (SEQ ID NO: 213), and the CDR3 sequence of ARYYVSGTYFPANY (SEQ ID NO: 246).

23. The VHH domain of any one of claims 1 to 22, wherein the VHH domain comprises a framework derived from the framework of any of the VHH domains comprising the sequences of
(SEQ ID NO: 93)
QVQLVESGGGLVQAGGSLKLACAAPGLTFSSYRMGWFRQAPGQEREFVA
AIDWNGRGTYYRYYADSVKGRSTISRDNAKNTMYLQMNSLKPEDTAVYY
CAAGSIDLNWYGGMDYWGQGTQVTVSS,
(SEQ ID NO: 94)
EVQVVESGGGLVQAGGSLKLACAAPGLTFSSYRMGWFRQAPGQEREFVA
AIDWNGRGTYYRYYADSVKGRSTISRDNAKNTVYLQMNSLKPEDTAVYY
CAATTVLTDPRVLNEYATWGQGTQVTVSS,
(SEQ ID NO: 95)
QLQLVESGGGLVQPGGSLRLSCAASGSIFSINVMGWYRQAPGKQRELVA
RINGGGITHYAESVKGRFTISRDNAKNTVYLQMNSLKPEDTAAYYCKAD
VFGSSGYVETYWGQGTQVTVSS,
(SEQ ID NO: 96)
EVQVVESGGGLVQAGGSLRLSCAVSGTSVSSNAMGWYRQAPGKQREWVG
FIDRIATTTIATSVKGRFAITRDNAKNTVYLQMSGLKPEDTAVYYCNHP
LTARWGQGTQVTVSS,
(SEQ ID NO: 97)
QVQLVESGGGLVQAGGSLRLSCAASGRTFSSYAMGWFRQAPGKEREFVA
AITWNGGTTYYADSVKGRFTISRDNAKNTVYLQMNSLKPEDTAVYYCAA
DPFNQGYWGQGTQVTVSS,
(SEQ ID NO: 98)
EVQLVESGGGLVQAGGSLRLSCAVSGSSVSSDAMGWYRQAPGNQRAWVA
FISGGGTTTYADSVKGRFTISRDNTKNTVYLHMNSLKPEDTAVYYCNHP
LTSRWGQGTQVTVSS,
(SEQ ID NO: 99)
EVQVVESGGGLVQAGGSLRLACVASRSIGSINVMGWYRQAPGKQRDLVA
RITGGGSTHYAESVKGRFTISRDNAKNTVYLQMNSLEPEDTAVYYCASM
VNPIITAWGTIGVREIPDYDYWGQGTQVTVSS,
(SEQ ID NO: 100)
QVQLVESGGGLVQAGGSLRLSCAVSGRTFSTYRMGWFRQAPGKERSFVA
AISWSGGSTTYADPVKGRFTISRDNAKNTVYLRMNSLKPEDTAVYYCND
QRGYWGQGTLVTVSS,
(SEQ ID NO: 101)
EVQVVESGGGLVQAGGSLRLSCAASGFTFTRYAMGWFRQAPGKERSFVA
AISWSGSSAGYGDSVKGRFTISRDNAKNTLYLQMNSLKPEDTAVYYCAA
DPFNQGYWGQGTQVTVSS,
(SEQ ID NO: 102)
EVQVVESGGGLVQAGGSLRLSCAASGRTFTTYRMGWFRQAPGKEREFVA
AIRWSGGRTLYADSVKGRFTISRDNAKNTAYLQMNNLRPEDTAVYYCAA
DLAEYSGTYSSPADSPAGYDYWGQGTQVTVSS,
or
(SEQ ID NO: 103)
QVQLVETGGGLVQAGDSLRLSCAASGRTLSFNTYAMGWFRQAPGKEREF
VASITWNGGSTSYADSVKGRFTITRDNAKNTATLRMNSLQPDDTAVYYC
AAARYYVSGTYFPANYWGQGTQVTVSS.

24. The VHH domain of any one of claims 1 to 22, wherein the VHH domain comprises a framework comprising sequence having at least 75%, 80%, 85%, 90%, 95%, 96%, 97%, 98%, 99%, or 100% sequence identity with the sequence of
(SEQ ID NO: 93)
QVQLVESGGGLVQAGGSLKLACAAPGLTFSSYRMGWFRQAPGQEREFVA
AIDWNGRGTYYRYYADSVKGRSTISRDNAKNTMYLQMNSLKPEDTAVYY
CAAGSIDLNWYGGMDYWGQGTQVTVSS,
(SEQ ID NO: 94)
EVQVVESGGGLVQAGGSLKLACAAPGLTFSSYRMGWFRQAPGQEREFVA
AIDWNGRGTYYRYYADSVKGRSTISRDNAKNTVYLQMNSLKPEDTAVYY
CAATTVLTDPRVLNEYATWGQGTQVTVSS,
(SEQ ID NO: 95)
QLQLVESGGGLVQPGGSLRLSCAASGSIFSINVMGWYRQAPGKQRELVA
RINGGGITHYAESVKGRFTISRDNAKNTVYLQMNSLKPEDTAAYYCKAD
VFGSSGYVETYWGQGTQVTVSS,
(SEQ ID NO: 96)
EVQVVESGGGLVQAGGSLRLSCAVSGTSVSSNAMGWYRQAPGKQREWVG
FIDRIATTTIATSVKGRFAITRDNAKNTVYLQMSGLKPEDTAVYYCNHP
LTARWGQGTQVTVSS,
(SEQ ID NO: 97)
QVQLVESGGGLVQAGGSLRLSCAASGRTFSSYAMGWFRQAPGKEREFVA
AITWNGGTTYYADSVKGRFTISRDNAKNTVYLQMNSLKPEDTAVYYCAA
DPFNQGYWGQGTQVTVSS,
(SEQ ID NO: 98)
EVQLVESGGGLVQAGGSLRLSCAVSGSSVSSDAMGWYRQAPGNQRAWVA
FISGGGTTTYADSVKGRFTISRDNTKNTVYLHMNSLKPEDTAVYYCNHP
LTSRWGQGTQVTVSS,
(SEQ ID NO: 99)
EVQVVESGGGLVQAGGSLRLACVASRSIGSINVMGWYRQAPGKQRDLVA
RITGGGSTHYAESVKGRFTISRDNAKNTVYLQMNSLEPEDTAVYYCASM
VNPIITAWGTIGVREIPDYDYWGQGTQVTVSS,
(SEQ ID NO: 100)
QVQLVESGGGLVQAGGSLRLSCAVSGRTFSTYRMGWFRQAPGKERSFVA
AISWSGGSTTYADPVKGRFTISRDNAKNTVYLRMNSLKPEDTAVYYCND
QRGYWGQGTLVTVSS,
(SEQ ID NO: 101)
EVQVVESGGGLVQAGGSLRLSCAASGFTFTRYAMGWFRQAPGKERSFVA
AISWSGSSAGYGDSVKGRFTISRDNAKNTLYLQMNSLKPEDTAVYYCAA
DPFNQGYWGQGTQVTVSS,
(SEQ ID NO: 102)
EVQVVESGGGLVQAGGSLRLSCAASGRTFTTYRMGWFRQAPGKEREFVA
AIRWSGGRTLYADSVKGRFTISRDNAKNTAYLQMNNLRPEDTAVYYCAA
DLAEYSGTYSSPADSPAGYDYWGQGTQVTVSS,
or
(SEQ ID NO: 103)
QVQLVETGGGLVQAGDSLRLSCAASGRTLSFNTYAMGWFRQAPGKEREF
VASITWNGGSTSYADSVKGRFTITRDNAKNTATLRMNSLQPDDTAVYYC
AAARYYVSGTYFPANYWGQGTQVTVSS.

25. The VHH domain of any one of claims 1 to 24, wherein the VHH domain is comprised of a sequence having at least 75%, 80%, 85%, 90%, 95%, 96%, 97%, 98%, 99%, or 100% sequence identity with the sequence of
(SEQ ID NO: 93)
QVQLVESGGGLVQAGGSLKLACAAPGLTFSSYRMGWFRQAPGQEREFVA
AIDWNGRGTYYRYYADSVKGRSTISRDNAKNTMYLQMNSLKPEDTAVYY
CAAGSIDLNWYGGMDYWGQGTQVTVSS,
(SEQ ID NO: 94)
EVQVVESGGGLVQAGGSLKLACAAPGLTFSSYRMGWFRQAPGQEREFVA
AIDWNGRGTYYRYYADSVKGRSTISRDNAKNTVYLQMNSLKPEDTAVYY
CAATTVLTDPRVLNEYATWGQGTQVTVSS,
(SEQ ID NO: 95)
QLQLVESGGGLVQPGGSLRLSCAASGSIFSINVMGWYRQAPGKQRELVA
RINGGGITHYAESVKGRFTISRDNAKNTVYLQMNSLKPEDTAAYYCKAD
VFGSSGYVETYWGQGTQVTVSS,
(SEQ ID NO: 96)
EVQVVESGGGLVQAGGSLRLSCAVSGTSVSSNAMGWYRQAPGKQREWVG
FIDRIATTTIATSVKGRFAITRDNAKNTVYLQMSGLKPEDTAVYYCNHP
LTARWGQGTQVTVSS,
(SEQ ID NO: 97)
QVQLVESGGGLVQAGGSLRLSCAASGRTFSSYAMGWFRQAPGKEREFVA
AITWNGGTTYYADSVKGRFTISRDNAKNTVYLQMNSLKPEDTAVYYCAA
DPFNQGYWGQGTQVTVSS,
(SEQ ID NO: 98)
EVQLVESGGGLVQAGGSLRLSCAVSGSSVSSDAMGWYRQAPGNQRAWVA
FISGGGTTTYADSVKGRFTISRDNTKNTVYLHMNSLKPEDTAVYYCNHP
LTSRWGQGTQVTVSS,
(SEQ ID NO: 99)
EVQVVESGGGLVQAGGSLRLACVASRSIGSINVMGWYRQAPGKQRDLVA
RITGGGSTHYAESVKGRFTISRDNAKNTVYLQMNSLEPEDTAVYYCASM
VNPIITAWGTIGVREIPDYDYWGQGTQVTVSS,
(SEQ ID NO: 100)
QVQLVESGGGLVQAGGSLRLSCAVSGRTFSTYRMGWFRQAPGKERSFVA
AISWSGGSTTYADPVKGRFTISRDNAKNTVYLRMNSLKPEDTAVYYCND
QRGYWGQGTLVTVSS,
(SEQ ID NO: 101)
EVQVVESGGGLVQAGGSLRLSCAASGFTFTRYAMGWFRQAPGKERSFVA
AISWSGSSAGYGDSVKGRFTISRDNAKNTLYLQMNSLKPEDTAVYYCAA
DPFNQGYWGQGTQVTVSS,
(SEQ ID NO: 102)
EVQVVESGGGLVQAGGSLRLSCAASGRTFTTYRMGWFRQAPGKEREFVA
AIRWSGGRTLYADSVKGRFTISRDNAKNTAYLQMNNLRPEDTAVYYCAA
DLAEYSGTYSSPADSPAGYDYWGQGTQVTVSS,
or
(SEQ ID NO: 103)
QVQLVETGGGLVQAGDSLRLSCAASGRTLSFNTYAMGWFRQAPGKEREF
VASITWNGGSTSYADSVKGRFTITRDNAKNTATLRMNSLQPDDTAVYYC
AAARYYVSGTYFPANYWGQGTQVTVSS.

26. An isolated nucleic acid molecule encoding the VHH domain of any of claims 1 to 25.

27. An isolated nucleic acid molecule encoding the VHH domain having a sequence with at least 85%, 90%, 95%, 96%, 97%, 98%, 99% or 100% sequence identity to the sequence of QVQLVESGGGLVQAGGSLKLACAAPGLTFSSYRMGWFRQAPGQEREFVAAIDWNGRG TYYRYYADSVKGRSTISRDNAKNTMYLQMNSLKPEDTAVYYCAAGSIDLNWYGGMD YWGQGTQVTVSS (SEQ ID NO: 93), EVQVVESGGGLVQAGGSLKLACAAPGLTFSSYRMGWFRQAPGQEREFVAAIDWNGRG TYYRYYADSVKGRSTISRDNAKNTVYLQMNSLKPEDTAVYYCAATTVLTDPRVLNEYA TWGQGTQVTVSS (SEQ ID NO: 94), QLQLVESGGGLVQPGGSLRLSCAASGSIFSINVMGWYRQAPGKQRELVARINGGGITHY AESVKGRFTISRDNAKNTVYLQMNSLKPEDTAAYYCKADVFGSSGYVETYWGQGTQV TVSS (SEQ ID NO: 95), EVQVVESGGGLVQAGGSLRLSCAVSGTSVSSNAMGWYRQAPGKQREWVGFIDRIATTT IATSVKGRFAITRDNAKNTVYLQMSGLKPEDTAVYYCNHPLTARWGQGTQVTVSS (SEQ ID NO: 96), QVQLVESGGGLVQAGGSLRLSCAASGRTFSSYAMGWFRQAPGKEREFVAAITWNGGTT YYADSVKGRFTISRDNAKNTVYLQMNSLKPEDTAVYYCAADPFNQGYWGQGTQVTVS S (SEQ ID NO: 97), EVQLVESGGGLVQAGGSLRLSCAVSGSSVSSDAMGWYRQAPGNQRAWVAFISGGGTT TYADSVKGRFTISRDNTKNTVYLHMNSLKPEDTAVYYCNHPLTSRWGQGTQVTVSS (SEQ ID NO: 98), EVQVVESGGGLVQAGGSLRLACVASRSIGSINVMGWYRQAPGKQRDLVARITGGGSTH YAESVKGRFTISRDNAKNTVYLQMNSLEPEDTAVYYCASMVNPIITAWGTIGVREIPDY DYWGQGTQVTVSS (SEQ ID NO: 99), QVQLVESGGGLVQAGGSLRLSCAVSGRTFSTYRMGWFRQAPGKERSFVAAISWSGGST TYADPVKGRFTISRDNAKNTVYLRMNSLKPEDTAVYYCNDQRGYWGQGTLVTVSS (SEQ ID NO: 100), EVQVVESGGGLVQAGGSLRLSCAASGFTFTRYAMGWFRQAPGKERSFVAAISWSGSSA GYGDSVKGRF TISRDNAKNTLYLQMNSLKPEDTAVYYCAADPFNQGYWGQGTQVTVS S (SEQ ID NO: 101), EVQVVESGGGLVQAGGSLRL SCAASGRTFTTYRMGWFRQAPGKEREFVAAIRWSGGRT LYADSVKGRF TISRDNAKNTAYLQMNNLRPED TAVYYCAADLAEYSGTYSSPADSPAG YDYWGQGTQVTVSS (SEQ ID NO: 102), or QVQLVETGGGLVQAGDSLRLSCAASGRTLSFNTYAMGWFRQAPGKEREFVASITWNG GSTSYADSVKGRFTITRDNAKNTATLRMNSLQPDDTAVYYCAAARYYVSGTYFPANY WGQGTQVTVSS (SEQ ID NO: 103), optionally wherein the nucleic acid molecule comprises a sequence with at least 85%, 90%, 95%, 96%, 97%, 98%, 99% or 100% sequence identity to the polynucleotide sequence of any one of SEQ ID NOS: 133-142.

28. A vector comprising the nucleic acid molecule of claim 26 or claim 27.

29. A cell expressing the nucleic acid molecule of claim 26 or claim 27.

30. A pharmaceutical composition comprising the VHH domain of any of claims 1 to 25 and a pharmaceutically acceptable excipient.

31. A pharmaceutical composition comprising a means for delivering a molecule to a mucosal lumen of a subject, and a pharmaceutically acceptable carrier.

32. A pharmaceutical composition comprising a means for delivering a molecule into systemic circulation in a subject, and a pharmaceutically acceptable carrier.

33. A pharmaceutical composition comprising a means for delivering a molecule into Lamina propria of a subject, and a pharmaceutically acceptable carrier.

34. A pharmaceutical composition comprising a means for delivering a molecule to an organ of a subject, and a pharmaceutically acceptable carrier.

35. A pharmaceutical composition comprising a means for delivering a molecule to a pIgR-expressing cell, and a pharmaceutically acceptable carrier.

36. The pharmaceutical composition of any one of claims 31 to 35, wherein the molecule is an antibody or fragment thereof, a peptide, a vaccine, a small molecule, a polynucleotide, a molecule comprising a radioactive isotope, a toxin, an enzyme, an anticoagulant, a hormone, a cytokine, an anti-inflammatory molecule, an RNAi, an mRNA, a self-replicating RNA, an antibiotic, or an antibody-antibiotic conjugate.

37. A therapeutic molecule comprising an agent and a VHH domain that binds to an extracellular domain of polymeric immunoglobulin receptor (pIgR).

38. The therapeutic molecule of claim 37, wherein the VHH domain binds to an extracellular domain 1 of pIgR.

39. The therapeutic molecule of claim 37, wherein the VHH domain binds to an extracellular domain 2 of pIgR.

40. The therapeutic molecule of claim 37, wherein the VHH domain binds to an extracellular domain 1-2 of pIgR.

41. The therapeutic molecule of claim 37, wherein the VHH domain binds to an extracellular domain 3 of pIgR.

42. The therapeutic molecule of claim 37, wherein the VHH domain binds to an extracellular domain 2-3 of pIgR.

43. The therapeutic molecule of claim 37, wherein the VHH domain binds to an extracellular domain 4-5 of pIgR.

44. The therapeutic molecule of claim 37, wherein the VHH domain binds to an extracellular domain 5 of pIgR.

45. The therapeutic molecule of any one of claims 37 to 44, wherein pIgR is human pIgR.

46. The therapeutic molecule of any one of claims 37 to 44, wherein pIgR is mouse pIgR.

47. The therapeutic molecule of any one of claims 37 to 44, wherein the VHH domain does not detectably bind to the amino acid sequence of
(SEQ ID NO: 143)
EKAVADTRDQADGSRASVDSGSSEEQGGSSR,
(SEQ ID NO: 144)
EREIQNVGDQAQENRASGDAGSADGQSRSSSSK,
or
(SEQ ID NO: 145)
EREIQNVRDQAQENRASGDAGSADGQSRSSSSK.

48. The therapeutic molecule of claim 37, wherein the VHH domain competes with IgA binding to the pIgR.

49. The therapeutic molecule of claim 37, wherein the VHH domain promotes IgA binding to the pIgR.

50. The therapeutic molecule of any one of claims 37 to 49, wherein the K_{D} of the binding of the VHH domain to pIgR is from about 4 to about 525 nM.

51. The therapeutic molecule of any one of claims 37 to 50, wherein the K_{D} of the binding of the VHH domain to pIgR is less than about 50 nM.

52. The therapeutic molecule of any one of claims 37 to 51, wherein the K_{D} of the binding of the VHH domain to pIgR is from about 4 to about 34 nM.

53. The therapeutic molecule of any one of claims 37 to 52, wherein the Tₘ of the VHH domain is from about 53 to about 77° C.

54. The therapeutic molecule of any one of claims 37 to 53, wherein the Tₘ of the VHH domain is from 53.9 to 76.4° C.

55. The therapeutic molecule of any one of claims 37 to 54, wherein the VHH domain comprises a CDR3 sequence of GSIDLNWYGGMDY (SEQ ID NO: 60), TTVLTDPRVLNEYAT (SEQ ID NO: 61), DVFGSSGYVETY (SEQ ID NO: 62), PLTAR (SEQ ID NO: 63), DPFNQGY (SEQ ID NO: 64), PLTSR (SEQ ID NO: 65), MVNPIITAWGTIGVREIPDYDY (SEQ ID NO: 66), DQRGY (SEQ ID NO: 67), QRGY (SEQ ID NO: 271), DPFNQGY (SEQ ID NO: 68), DLAEYSGTYSSPADSPAGYDY (SEQ ID NO: 69), ARYYVSGTYFPANY (SEQ ID NO: 70), GSIDLNWYGGMDY (SEQ ID NO: 71), SIDLNWYGGMD (SEQ ID NO: 272), TTVLTDPRVLNEYAT (SEQ ID NO: 72), TVLTDPRVLNEYA (SEQ ID NO: 273), DVFGSSGYVETY (SEQ ID NO: 73), VFGSSGYVET (SEQ ID NO: 274), PLTAR (SEQ ID NO: 74), LTA (SEQ ID NO: 275), DPFNQGY (SEQ ID NO: 75), PFNQG (SEQ ID NO: 276), PLTSR (SEQ ID NO: 76), LTS (SEQ ID NO: 277), MVNPIITAWGTIGVREIPDYDY (SEQ ID NO: 77), VNPIITAWGTIGVREIPDYD (SEQ ID NO: 278), DQRGY (SEQ ID NO: 78), RG (SEQ ID NO: 279), DPFNQGY (SEQ ID NO: 79), PFNQG (SEQ ID NO: 280), DLAEYSGTYSSPADSPAGYDY (SEQ ID NO: 80), LAEYSGTYSSPADSPAGYD (SEQ ID NO: 281), ARYYVSGTYFPANY (SEQ ID NO: 81), RYYVSGTYFPAN (SEQ ID NO: 282), CAAGSIDLNWYGGMDY (SEQ ID NO: 82), AAGSIDLNWYGGMDY (SEQ ID NO: 283), CAATTVLTDPRVLNEYAT (SEQ ID NO: 83), AATTVLTDPRVLNEYAT (SEQ ID NO: 284), KADVFGSSGYVETY (SEQ ID NO: 84), NHPLTAR (SEQ ID NO: 85), AADPFNQGY (SEQ ID NO: 86), NHPLTSR (SEQ ID NO: 87), ASMVNPIITAWGTIGVREIPDYDY (SEQ ID NO: 88), NDQRGY (SEQ ID NO: 89), AADPFNQGY (SEQ ID NO: 90), AADLAEYSGTYSSPADSPAGYDY (SEQ ID NO: 91), AAARYYVSGTYFPANY (SEQ ID NO: 92), GSIDLNWYGGMDY (SEQ ID NO: 214), TTVLTDPRVLNEYAT (SEQ ID NO: 215), DVFGSSGYVETY (SEQ ID NO: 216), PLTAR (SEQ ID NO: 217), DPFNQGY (SEQ ID NO: 218), PLTSR (SEQ ID NO: 219), MVNPIITAWGTIGVREIPDYDY (SEQ ID NO: 220), QRGY (SEQ ID NO: 221), DPFNQGY (SEQ ID NO: 222), DLAEYSGTYSSPADSPAGYDY (SEQ ID NO: 223), ARYYVSGTYFPANY (SEQ ID NO: 224), AAGSIDLNWYGGMD (SEQ ID NO: 225), AATTVLTDPRVLNEYA (SEQ ID NO: 226), KADVFGSSGYVET (SEQ ID NO: 227), NHPLTA (SEQ ID NO: 228), AADPFNQG (SEQ ID NO: 229), NHPLTS (SEQ ID NO: 230), ASMVNPIITAWGTIGVREIPDYD (SEQ ID NO: 231), NDQRG (SEQ ID NO: 232), AADPFNQG (SEQ ID NO: 233), AADLAEYSGTYSSPADSPAGYD (SEQ ID NO: 234), AAARYYVSGTYFPAN (SEQ ID NO: 235), GSIDLNWYGGMDY (SEQ ID NO: 236), TTVLTDPRVLNEYAT (SEQ ID NO: 237), DVFGSSGYVETY (SEQ ID NO: 238), PLTAR (SEQ ID NO: 239), DPFNQGY (SEQ ID NO: 240), PLTSR (SEQ ID NO: 241), MVNPIITAWGTIGVREIPDYDY (SEQ ID NO: 242), QRGY (SEQ ID NO: 243), DPFNQGY (SEQ ID NO: 244), DLAEYSGTYSSPADSPAGYDY (SEQ ID NO: 245), or ARYYVSGTYFPANY (SEQ ID NO: 246).

56. The therapeutic molecule of any one of claims 37 to 55, wherein the VHH domain comprises a CDR2 sequence of AIDWNGRGTYYRYYADSVKG (SEQ ID NO: 30), RINGGGITHYAESVKG (SEQ ID NO: 31), FIDRIATTTIATSVKG (SEQ ID NO: 32), AITWNGGTTYYADSVKG (SEQ ID NO: 33), FISGGGTTTYADSVKG (SEQ ID NO: 34), RITGGGSTHYAESVKG (SEQ ID NO: 35), AISWSGGSTTYADPVKG (SEQ ID NO: 36), AISWSGSSAGYGDSVKG (SEQ ID NO: 37), AIRWSGGRTLYADSVKG (SEQ ID NO: 38), SITWNGGSTSYADSVKG (SEQ ID NO: 39), DWNGRGTYY (SEQ ID NO: 40), WNGRGTY (SEQ ID NO: 260), NGGGI (SEQ ID NO: 41), GGG (SEQ ID NO: 261), DRIAT (SEQ ID NO: 42), RIA (SEQ ID NO: 262), TWNGGT (SEQ ID NO: 43), WNGG (SEQ ID NO: 263), SGGGT (SEQ ID NO: 44), GGG (SEQ ID NO: 264), TGGGS (SEQ ID NO: 45), GGG (SEQ ID NO: 265), SWSGGS (SEQ ID NO: 46), WSGG (SEQ ID NO: 266), SWSGSS (SEQ ID NO: 47), WSGS (SEQ ID NO: 267), RWSGGR (SEQ ID NO: 48), WSGG (SEQ ID NO: 268), TWNGGS (SEQ ID NO: 49), WNGG (SEQ ID NO: 269), IDWNGRGTYY (SEQ ID NO: 50), IDWNGRGTYYR (SEQ ID NO: 270), INGGGIT (SEQ ID NO: 51), IDRIATT (SEQ ID NO: 52), ITWNGGTT (SEQ ID NO: 53), ISGGGTT (SEQ ID NO: 54), ITGGGST (SEQ ID NO: 55), ISWSGGST (SEQ ID NO: 56), ISWSGSSA (SEQ ID NO: 57), IRWSGGRT (SEQ ID NO: 58), ITWNGGST (SEQ ID NO: 59), AIDWNGRGTYYRYYADSVKG (SEQ ID NO: 184), RINGGGITHYAESVKG (SEQ ID NO: 185), FIDRIATTTIATSVKG (SEQ ID NO: 186), AITWNGGTTYYADSVKG (SEQ ID NO: 187), FISGGGTTTYADSVKG (SEQ ID NO: 188), RITGGGSTHYAESVKG (SEQ ID NO: 189), AISWSGGSTTYADPVKG (SEQ ID NO: 190), AISWSGSSAGYGDSVKG (SEQ ID NO: 191), AIRWSGGRTLYADSVKG (SEQ ID NO: 192), SITWNGGSTSYADSVKG (SEQ ID NO: 193), FVAAIDWNGRGTYYRY (SEQ ID NO: 194), LVARINGGGITH (SEQ ID NO: 195), WVGFIDRIATTT (SEQ ID NO: 196), FVAAITWNGGTTY (SEQ ID NO: 197), WVAFISGGGTTT (SEQ ID NO: 198), LVARITGGGSTH (SEQ ID NO: 199), FVAAISWSGGSTT (SEQ ID NO: 200), FVAAISWSGSSAG (SEQ ID NO: 201), FVAAIRWSGGRTL (SEQ ID NO: 202), FVASITWNGGSTS (SEQ ID NO: 203), AIDWNGRGTYYRY (SEQ ID NO: 204), RINGGGITH (SEQ ID NO: 205), FIDRIATTT (SEQ ID NO: 206), AITWNGGTTY (SEQ ID NO: 207), FISGGGTTT (SEQ ID NO: 208), RITGGGSTH (SEQ ID NO: 209), AISWSGGSTT (SEQ ID NO: 210), AISWSGSSAG (SEQ ID NO: 211), AIRWSGGRTL (SEQ ID NO: 212), or SITWNGGSTS (SEQ ID NO: 213).

57. The therapeutic molecule of any one of claims 37 to 56, wherein the VHH domain comprises a CDR1 sequence of SYRMG (SEQ ID NO: 1), INVMG (SEQ ID NO: 2), SNAMG (SEQ ID NO: 3), SYAMG (SEQ ID NO: 4), SDAMG (SEQ ID NO: 5), INVMG (SEQ ID NO: 6), TYRMG (SEQ ID NO: 7), RYAMG (SEQ ID NO: 8), FTTYRMG (SEQ ID NO: 258), TYRMG (SEQ ID NO: 259), FNTYAMG (SEQ ID NO: 9), GLTFSSY (SEQ ID NO: 10), GSIFSIN (SEQ ID NO: 11), GTSVSSN (SEQ ID NO: 12), GRTFSSY (SEQ ID NO: 13), GSSVSSD (SEQ ID NO: 14), RSIGSIN (SEQ ID NO: 15), GRTFSTY (SEQ ID NO: 16), GFTFTRY (SEQ ID NO: 17), GRTFTTY (SEQ ID NO: 18), GRTLSFNTY (SEQ ID NO: 19), GLTFSSYR (SEQ ID NO: 20), GSIFSINV (SEQ ID NO: 21), GTSVSSNA (SEQ ID NO: 22), GRTFSSYA (SEQ ID NO: 23), GSSVSSDA (SEQ ID NO: 24), RSIGSINV (SEQ ID NO: 25), GRTFSTYR (SEQ ID NO: 26), GFTFTRYA (SEQ ID NO: 27), GRTFTTYR (SEQ ID NO: 28), GRTLSFNTYA (SEQ ID NO: 29), GLTFSSYRMG (SEQ ID NO: 154), GSIFSINVMG (SEQ ID NO: 155), GTSVSSNAMG (SEQ ID NO: 156), GRTFSSYAMG (SEQ ID NO: 157), GSSVSSDAMG (SEQ ID NO: 158), RSIGSINVMG (SEQ ID NO: 159), GRTFSTYRMG (SEQ ID NO: 160), GFTFTRYAMG (SEQ ID NO: 161), GRTFTTYRMG (SEQ ID NO: 162), GRTLSFNTYAMG (SEQ ID NO: 163), SSYRMG (SEQ ID NO: 164), SINVMG (SEQ ID NO: 165), SSNAMG (SEQ ID NO: 166), SSYAMG (SEQ ID NO: 167), SSDAMG (SEQ ID NO: 168), SINVMG (SEQ ID NO: 169), STYRMG (SEQ ID NO: 170), TRYAMG (SEQ ID NO: 171), TTYRMG (SEQ ID NO: 172), SFNTYAMG (SEQ ID NO: 173), GLTFSSYRMG (SEQ ID NO: 174), GSIFSINVMG (SEQ ID NO: 175), GTSVSSNAMG (SEQ ID NO: 176), GRTFSSYAMG (SEQ ID NO: 177), GSSVSSDAMG (SEQ ID NO: 178), RSIGSINVMG (SEQ ID NO: 179), GRTFSTYRMG (SEQ ID NO: 180), GFTFTRYAMG (SEQ ID NO: 181), GRTFTTYRMG (SEQ ID NO: 182), or GRTLSFNTYAMG (SEQ ID NO: 183).

58. The therapeutic molecule of any one of claims 37 to 57, wherein the VHH domain comprises a CDR1 sequence, a CDR2 sequence, and a CDR3 sequence of the VHH domain selected from the group consisting of:
a) VHH1:
i) the CDR1 sequence of SYRMG (SEQ ID NO: 1), the CDR2 sequence of AIDWNGRGTYYRYYADSVKG (SEQ ID NO: 30), and the CDR3 sequence of GSIDLNWYGGMDY (SEQ ID NO: 60);
ii) the CDR1 sequence of GLTFSSY (SEQ ID NO: 10), the CDR2 sequence of DWNGRGTYY (SEQ ID NO: 40) or WNGRGTY (SEQ ID NO: 260), and the CDR3 sequence of GSIDLNWYGGMDY (SEQ ID NO: 71) or SIDLNWYGGMD (SEQ ID NO: 272);
iii) the CDR1 sequence of GLTFSSYR (SEQ ID NO: 20), the CDR2 sequence of IDWNGRGTYY (SEQ ID NO: 50) or IDWNGRGTYYR (SEQ ID NO: 270), and the CDR3 sequence of CAAGSIDLNWYGGMDY (SEQ ID NO: 82) or AAGSIDLNWYGGMDY (SEQ ID NO: 283);
iv) the CDR1 sequence of GLTFSSYRMG (SEQ ID NO: 154), the CDR 2 sequence of AIDWNGRGTYYRYYADSVKG (SEQ ID NO: 184), and the CDR3 sequence of GSIDLNWYGGMDY (SEQ ID NO: 214);
v) the CDR1 sequence of SSYRMG (SEQ ID NO: 164), the CDR2 sequence of FVAAIDWNGRGTYYRY (SEQ ID NO: 194), and the CDR3 sequence of AAGSIDLNWYGGMD (SEQ ID NO: 225); or
vi) the CDR1 sequence of GLTFSSYRMG (SEQ ID NO: 174), the CDR2 sequence of AIDWNGRGTYYRY (SEQ ID NO: 204), and the CDR3 sequence of GSIDLNWYGGMDY (SEQ ID NO: 236);
b) VHH2:
i) the CDR1 sequence of SYRMG (SEQ ID NO: 1), the CDR2 sequence of AIDWNGRGTYYRYYADSVKG (SEQ ID NO: 30), and the CDR3 sequence of TTVLTDPRVLNEYAT (SEQ ID NO: 61);
ii) the CDR1 sequence of GLTFSSY (SEQ ID NO: 10), the CDR2 sequence of DWNGRGTYY (SEQ ID NO: 40) or WNGRGTY (SEQ ID NO: 260), and the CDR3 sequence of TTVLTDPRVLNEYAT (SEQ ID NO: 72) or TVLTDPRVLNEYA (SEQ ID NO: 273);
iii) the CDR1 sequence of GLTFSSYR (SEQ ID NO: 20), the CDR2 sequence of IDWNGRGTYY (SEQ ID NO: 50) or IDWNGRGTYYR (SEQ ID NO: 270), and the CDR3 sequence of CAATTVLTDPRVLNEYAT (SEQ ID NO: 83) or AATTVLTDPRVLNEYAT (SEQ ID NO: 284);
iv) the CDR1 sequence of GLTFSSYRMG (SEQ ID NO: 154), the CDR2 sequence of AIDWNGRGTYYRYYADSVKG (SEQ ID NO: 184), and the CDR3 sequence of TTVLTDPRVLNEYAT (SEQ ID NO: 215);
v) the CDR1 sequence of SSYRMG (SEQ ID NO: 164), the CDR2 sequence of FVAAIDWNGRGTYYRY (SEQ ID NO: 194), and the CDR3 sequence of AATTVLTDPRVLNEYA (SEQ ID NO: 226); or
vi) the CDR1 sequence of GLTFSSYRMG (SEQ ID NO: 174), the CDR2 sequence of AIDWNGRGTYYRY (SEQ ID NO: 204), and the CDR3 sequence of TTVLTDPRVLNEYAT (SEQ ID NO: 237);
c) VHH3:
i) the CDR1 sequence of INVMG (SEQ ID NO: 2), the CDR2 sequence of RINGGGITHYAESVKG (SEQ ID NO: 31), and the CDR3 sequence of DVFGSSGYVETY (SEQ ID NO: 62);
ii) the CDR1 sequence of GSIFSIN (SEQ ID NO: 11), the CDR2 sequence of NGGGI (SEQ ID NO: 41) or GGG (SEQ ID NO: 261), and the CDR3 sequence of DVFGSSGYVETY (SEQ ID NO: 73) or VFGSSGYVET (SEQ ID NO: 274);
iii) the CDR1 sequence of GSIFSINV (SEQ ID NO: 21), the CDR2 sequence of INGGGIT (SEQ ID NO: 51), and the CDR3 sequence of KADVFGSSGYVETY (SEQ ID NO: 84);
iv) the CDR1 sequence of GSIFSINVMG (SEQ ID NO: 155), the CDR2 sequence of RINGGGITHYAESVKG (SEQ ID NO: 185), and the CDR3 sequence of DVFGSSGYVETY (SEQ ID NO: 216);
v) the CDR1 sequence of SINVMG (SEQ ID NO: 165), the CDR2 sequence of LVARINGGGITH (SEQ ID NO: 195), and the CDR3 sequence of KADVFGSSGYVET (SEQ ID NO: 227); or
vi) the CDR1 sequence of GSIFSINVMG (SEQ ID NO: 175), the CDR2 sequence of RINGGGITH (SEQ ID NO: 205), and the CDR3 sequence of DVFGSSGYVETY (SEQ ID NO: 238);
d) VHH4:
i) the CDR1 sequence of SNAMG (SEQ ID NO: 3), the CDR2 sequence of FIDRIATTTIATSVKG (SEQ ID NO: 32), and the CDR3 sequence of PLTAR (SEQ ID NO: 63);
ii) the CDR1 sequence of GTSVSSN (SEQ ID NO: 12), the CDR2 sequence of DRIAT (SEQ ID NO: 42) or RIA (SEQ ID NO: 262), and the CDR3 sequence of PLTAR (SEQ ID NO: 74) or LTA (SEQ ID NO: 275);
iii) the CDR1 sequence of GTSVSSNA (SEQ ID NO: 22), the CDR2 sequence of IDRIATT (SEQ ID NO: 52), and the CDR3 sequence of NHPLTAR (SEQ ID NO: 85);
iv) the CDR1 sequence of GTSVSSNAMG (SEQ ID NO: 156), the CDR2 sequence of FIDRIATTTIATSVKG (SEQ ID NO: 186), and the CDR3 sequence of PLTAR (SEQ ID NO: 217);
v) the CDR1 sequence of SSNAMG (SEQ ID NO: 166), the CDR2 sequence of WVGFIDRIATTT (SEQ ID NO: 196), and the CDR3 sequence of NHPLTA (SEQ ID NO: 228); or
vi) the CDR1 sequence of GTSVSSNAMG (SEQ ID NO: 176), the CDR2 sequence of FIDRIATTT (SEQ ID NO: 206), and the CDR3 sequence of PLTAR (SEQ ID NO: 239);
e) VHH5:
i) the CDR1 sequence of SYAMG (SEQ ID NO: 4), the CDR2 sequence of AITWNGGTTYYADSVKG (SEQ ID NO: 33), and the CDR3 sequence of DPFNQGY (SEQ ID NO: 64);
ii) the CDR1 sequence of GRTFSSY (SEQ ID NO: 13), the CDR2 sequence of TWNGGT (SEQ ID NO: 43) or WNGG (SEQ ID NO: 263), and the CDR3 sequence of DPFNQGY (SEQ ID NO: 75) or PFNQG (SEQ ID NO: 276);
iii) the CDR1 sequence of GRTFSSYA (SEQ ID NO: 23), the CDR2 sequence of ITWNGGTT (SEQ ID NO: 53), and the CDR3 sequence of AADPFNQGY (SEQ ID NO: 86);
iv) the CDR1 sequence of GRTFSSYAMG (SEQ ID NO: 157), the CDR2 sequence of AITWNGGTTYYADSVKG (SEQ ID NO: 187), and the CDR3 sequence of DPFNQGY (SEQ ID NO: 218);
v) the CDR1 sequence of SSYAMG (SEQ ID NO: 167), the CDR2 sequence of FVAAITWNGGTTY (SEQ ID NO: 197), and the CDR3 sequence of AADPFNQG (SEQ ID NO: 229); or
vi) the CDR1 sequence of GRTFSSYAMG (SEQ ID NO: 177), the CDR2 sequence of AITWNGGTTY (SEQ ID NO: 207), and the CDR3 sequence of DPFNQGY (SEQ ID NO: 240);
f) VHH6:
i) the CDR1 sequence of SDAMG (SEQ ID NO: 5), the CDR2 sequence of FISGGGTTTYADSVKG (SEQ ID NO: 34), and the CDR3 sequence of PLTSR (SEQ ID NO: 65);
ii) the CDR1 sequence of GSSVSSD (SEQ ID NO: 14), the CDR2 sequence of SGGGT (SEQ ID NO: 44) or GGG (SEQ ID NO: 264), and the CDR3 sequence of PLTSR (SEQ ID NO: 76) or LTS (SEQ ID NO: 277);
iii) the CDR1 sequence of GSSVSSDA (SEQ ID NO: 24), the CDR2 sequence of ISGGGTT (SEQ ID NO: 54), and the CDR3 sequence of NHPLTSR (SEQ ID NO: 87);
iv) the CDR1 sequence of GSSVSSDAMG (SEQ ID NO: 158), the CDR2 sequence of FISGGGTTTYADSVKG (SEQ ID NO: 188), and the CDR3 sequence of PLTSR (SEQ ID NO: 219);
v) the CDR1 sequence of SSDAMG (SEQ ID NO: 168), the CDR2 sequence of WVAFISGGGTTT (SEQ ID NO: 198), and the CDR3 sequence of NHPLTS (SEQ ID NO: 230); or
vi) the CDR1 sequence of GSSVSSDAMG (SEQ ID NO: 178), the CDR2 sequence of FISGGGTTT (SEQ ID NO: 208), and the CDR3 sequence of PLTSR (SEQ ID NO: 241);
g) VHH7:
i) the CDR1 sequence of INVMG (SEQ ID NO: 6), the CDR2 sequence of RITGGGSTHYAESVKG (SEQ ID NO: 35), and the CDR3 sequence of MVNPIITAWGTIGVREIPDYDY (SEQ ID NO: 66);
ii) the CDR1 sequence of RSIGSIN (SEQ ID NO: 15), the CDR2 sequence of TGGGS (SEQ ID NO: 45) or GGG (SEQ ID NO: 265), and the CDR3 sequence of MVNPIITAWGTIGVREIPDYDY (SEQ ID NO: 77) or VNPIITAWGTIGVREIPDYD (SEQ ID NO: 278);
iii) the CDR1 sequence of RSIGSINV (SEQ ID NO: 25), the CDR2 sequence of ITGGGST (SEQ ID NO: 55), and the CDR3 sequence of ASMVNPIITAWGTIGVREIPDYDY (SEQ ID NO: 88);
iv) the CDR1 sequence of RSIGSINVMG (SEQ ID NO: 159), the CDR2 sequence of RITGGGSTHYAESVKG (SEQ ID NO: 189), and the CDR3 sequence of MVNPIITAWGTIGVREIPDYDY (SEQ ID NO: 220);
v) the CDR1 sequence of SINVMG (SEQ ID NO: 169), the CDR2 sequence of LVARITGGGSTH (SEQ ID NO: 199), and the CDR3 sequence of ASMVNPIITAWGTIGVREIPDYD (SEQ ID NO: 231); or
vi) the CDR1 sequence of RSIGSINVMG (SEQ ID NO: 179), the CDR2 sequence of RITGGGSTH (SEQ ID NO: 209), and the CDR3 sequence of MVNPIITAWGTIGVREIPDYDY (SEQ ID NO: 242);
h) VHH9:
i) the CDR1 sequence of TYRMG (SEQ ID NO: 7), the CDR2 sequence of AISWSGGSTTYADPVKG (SEQ ID NO: 36), and the CDR3 sequence of DQRGY (SEQ ID NO: 67) or QRGY (SEQ ID NO: 271);
ii) the CDR1 sequence of GRTFSTY (SEQ ID NO: 16), the CDR2 sequence of SWSGGS (SEQ ID NO: 46) or WSGG (SEQ ID NO: 266), and the CDR3 sequence of DQRGY (SEQ ID NO: 78) or RG (SEQ ID NO: 279);
iii) the CDR1 sequence of GRTFSTYR (SEQ ID NO: 26), the CDR2 sequence of ISWSGGST (SEQ ID NO: 56), and the CDR3 sequence of NDQRGY (SEQ ID NO: 89);
iv) the CDR1 sequence of GRTFSTYRMG (SEQ ID NO: 160), the CDR2 sequence of AISWSGGSTTYADPVKG (SEQ ID NO: 190), and the CDR3 sequence of QRGY (SEQ ID NO: 221);
v) the CDR1 sequence of STYRMG (SEQ ID NO: 170), the CDR2 sequence of FVAAISWSGGSTT (SEQ ID NO: 200), and the CDR3 sequence of NDQRG (SEQ ID NO: 232); or
vi) the CDR1 sequence of GRTFSTYRMG (SEQ ID NO: 180), the CDR2 sequence of AISWSGGSTT (SEQ ID NO: 210), and the CDR3 sequence of QRGY (SEQ ID NO: 243);
i) VHH10:
i) the CDR1 sequence of RYAMG (SEQ ID NO: 8), the CDR2 sequence of AISWSGSSAGYGDSVKG (SEQ ID NO: 37), and the CDR3 sequence of DPFNQGY (SEQ ID NO: 68);
ii) the CDR1 sequence of GFTFTRY (SEQ ID NO: 17), the CDR2 sequence of SWSGSS (SEQ ID NO: 47) or WSGS (SEQ ID NO: 267), and the CDR3 sequence of DPFNQGY (SEQ ID NO: 79) or PFNQG (SEQ ID NO: 280);
iii) the CDR1 sequence of GFTFTRYA (SEQ ID NO: 27), the CDR2 sequence of ISWSGSSA (SEQ ID NO: 57), and the CDR3 sequence of AADPFNQGY (SEQ ID NO: 90);
iv) the CDR1 sequence of GFTFTRYAMG (SEQ ID NO: 161), the CDR2 sequence of AISWSGSSAGYGDSVKG (SEQ ID NO: 191), and the CDR3 sequence of DPFNQGY (SEQ ID NO: 222);
v) the CDR1 sequence of TRYAMG (SEQ ID NO: 171), the CDR2 sequence of FVAAISWSGSSAG (SEQ ID NO: 201), and the CDR3 sequence of AADPFNQG (SEQ ID NO: 233); or
vi) the CDR1 sequence of GFTFTRYAMG (SEQ ID NO: 181), the CDR2 sequence of AISWSGSSAG (SEQ ID NO: 211), and the CDR3 sequence of DPFNQGY (SEQ ID NO: 244);
j) VHH11:
i) the CDR1 sequence of FTTYRMG (SEQ ID NO: 258) or TYRMG (SEQ ID NO: 259), the CDR2 sequence of AIRWSGGRTLYADSVKG (SEQ ID NO: 38), and the CDR3 sequence of DLAEYSGTYSSPADSPAGYDY (SEQ ID NO: 69);
ii) the CDR1 sequence of GRTFTTY (SEQ ID NO: 18), the CDR2 sequence of RWSGGR (SEQ ID NO: 48) or WSGG (SEQ ID NO: 268), and the CDR3 sequence of DLAEYSGTYSSPADSPAGYDY (SEQ ID NO: 80) or LAEYSGTYSSPADSPAGYD (SEQ ID NO: 281);
iii) the CDR1 sequence of GRTFTTYR (SEQ ID NO: 28), the CDR2 sequence of IRWSGGRT (SEQ ID NO: 58), and the CDR3 sequence of AADLAEYSGTYSSPADSPAGYDY (SEQ ID NO: 91);
iv) the CDR1 sequence of GRTFTTYRMG (SEQ ID NO: 162), the CDR2 sequence of AIRWSGGRTLYADSVKG (SEQ ID NO: 192), and the CDR3 sequence of DLAEYSGTYSSPADSPAGYDY (SEQ ID NO: 223);
v) the CDR1 sequence of TTYRMG (SEQ ID NO: 172), the CDR2 sequence of FVAAIRWSGGRTL (SEQ ID NO: 202), and the CDR3 sequence of AADLAEYSGTYSSPADSPAGYD (SEQ ID NO: 234); or
vi) the CDR1 sequence of GRTFTTYRMG (SEQ ID NO: 182), the CDR2 sequence of AIRWSGGRTL (SEQ ID NO: 212), and the CDR3 sequence of DLAEYSGTYSSPADSPAGYDY (SEQ ID NO: 245); and
k) VHH12:
i) the CDR1 sequence of FNTYAMG (SEQ ID NO: 9), the CDR2 sequence of SITWNGGSTSYADSVKG (SEQ ID NO: 39), and the CDR3 sequence of ARYYVSGTYFPANY (SEQ ID NO: 70);
ii) the CDR1 sequence of GRTLSFNTY (SEQ ID NO: 19), the CDR2 sequence of TWNGGS (SEQ ID NO: 49) or WNGG (SEQ ID NO: 269), and the CDR3 sequence of ARYYVSGTYFPANY (SEQ ID NO: 81) or RYYVSGTYFPAN (SEQ ID NO: 282);
iii) the CDR1 sequence of GRTLSFNTYA (SEQ ID NO: 29), the CDR2 sequence of ITWNGGST (SEQ ID NO: 59), and the CDR3 sequence of AAARYYVSGTYFPANY (SEQ ID NO: 92);
iv) the CDR1 sequence of GRTLSFNTYAMG (SEQ ID NO: 163), the CDR2 sequence of SITWNGGSTSYADSVKG (SEQ ID NO: 193), and the CDR3 sequence of ARYYVSGTYFPANY (SEQ ID NO: 224);
v) the CDR1 sequence of SFNTYAMG (SEQ ID NO: 173), the CDR2 sequence of FVASITWNGGSTS (SEQ ID NO: 203), and the CDR3 sequence of AAARYYVSGTYFPAN (SEQ ID NO: 235); or
vi) the CDR1 sequence of GRTLSFNTYAMG (SEQ ID NO: 183), the CDR2 sequence of SITWNGGSTS (SEQ ID NO: 213), and the CDR3 sequence of ARYYVSGTYFPANY (SEQ ID NO: 246).

59. The therapeutic molecule of any one of claims 37 to 58, wherein the VHH domain comprises a framework derived from the framework of any of the VHH domains comprising the sequences of
(SEQ ID NO: 93)
QVQLVESGGGLVQAGGSLKLACAAPGLTFSSYRMGWFRQAPGQEREFVA
AIDWNGRGTYYRYYADSVKGRSTISRDNAKNTMYLQMNSLKPEDTAVYY
CAAGSIDLNWYGGMDYWGQGTQVTVSS,
(SEQ ID NO: 94)
EVQVVESGGGLVQAGGSLKLACAAPGLTFSSYRMGWFRQAPGQEREFVA
AIDWNGRGTYYRYYADSVKGRSTISRDNAKNTVYLQMNSLKPEDTAVYY
CAATTVLTDPRVLNEYATWGQGTQVTVSS,
(SEQ ID NO: 95)
QLQLVESGGGLVQPGGSLRLSCAASGSIFSINVMGWYRQAPGKQRELVA
RINGGGITHYAESVKGRFTISRDNAKNTVYLQMNSLKPEDTAAYYCKAD
VFGSSGYVETYWGQGTQVTVSS,
(SEQ ID NO: 96)
EVQVVESGGGLVQAGGSLRLSCAVSGTSVSSNAMGWYRQAPGKQREWVG
FIDRIATTTIATSVKGRFAITRDNAKNTVYLQMSGLKPEDTAVYYCNHP
LTARWGQGTQVTVSS,
(SEQ ID NO: 97)
QVQLVESGGGLVQAGGSLRLSCAASGRTFSSYAMGWFRQAPGKEREFVA
AITWNGGTTYYADSVKGRFTISRDNAKNTVYLQMNSLKPEDTAVYYCAA
DPFNQGYWGQGTQVTVSS,
(SEQ ID NO: 98)
EVQLVESGGGLVQAGGSLRLSCAVSGSSVSSDAMGWYRQAPGNQRAWVA
FISGGGTTTYADSVKGRFTISRDNTKNTVYLHMNSLKPEDTAVYYCNHP
LTSRWGQGTQVTVSS,
(SEQ ID NO: 99)
EVQVVESGGGLVQAGGSLRLACVASRSIGSINVMGWYRQAPGKQRDLVA
RITGGGSTHYAESVKGRFTISRDNAKNTVYLQMNSLEPEDTAVYYCASM
VNPIITAWGTIGVREIPDYDYWGQGTQVTVSS,
(SEQ ID NO: 100)
QVQLVESGGGLVQAGGSLRLSCAVSGRTFSTYRMGWFRQAPGKERSFVA
AISWSGGSTTYADPVKGRFTISRDNAKNTVYLRMNSLKPEDTAVYYCND
QRGYWGQGTLVTVSS,
(SEQ ID NO: 101)
EVQVVESGGGLVQAGGSLRLSCAASGFTFTRYAMGWFRQAPGKERSFVA
AISWSGSSAGYGDSVKGRFTISRDNAKNTLYLQMNSLKPEDTAVYYCAA
DPFNQGYWGQGTQVTVSS,
(SEQ ID NO: 102)
EVQVVESGGGLVQAGGSLRLSCAASGRTFTTYRMGWFRQAPGKEREFVA
AIRWSGGRTLYADSVKGRFTISRDNAKNTAYLQMNNLRPEDTAVYYCAA
DLAEYSGTYSSPADSPAGYDYWGQGTQVTVSS,
or
(SEQ ID NO: 103)
QVQLVETGGGLVQAGDSLRLSCAASGRTLSFNTYAMGWFRQAPGKEREF
VASITWNGGSTSYADSVKGRFTITRDNAKNTATLRMNSLQPDDTAVYYC
AAARYYVSGTYFPANYWGQGTQVTVSS.

60. The therapeutic molecule of any one of claims 37 to 58, wherein the VHH domain comprises a framework comprising sequence having at least 75%, 80%, 85%, 90%, 95%, 96%, 97%, 98%, 99%, or 100% sequence identity with the sequence of
(SEQ ID NO: 93)
QVQLVESGGGLVQAGGSLKLACAAPGLTFSSYRMGWFRQAPGQEREFVA
AIDWNGRGTYYRYYADSVKGRSTISRDNAKNTMYLQMNSLKPEDTAVYY
CAAGSIDLNWYGGMDYWGQGTQVTVSS,
(SEQ ID NO: 94)
EVQVVESGGGLVQAGGSLKLACAAPGLTFSSYRMGWFRQAPGQEREFVA
AIDWNGRGTYYRYYADSVKGRSTISRDNAKNTVYLQMNSLKPEDTAVYY
CAATTVLTDPRVLNEYATWGQGTQVTVSS,
(SEQ ID NO: 95)
QLQLVESGGGLVQPGGSLRLSCAASGSIFSINVMGWYRQAPGKQRELVA
RINGGGITHYAESVKGRFTISRDNAKNTVYLQMNSLKPEDTAAYYCKAD
VFGSSGYVETYWGQGTQVTVSS,
(SEQ ID NO: 96)
EVQVVESGGGLVQAGGSLRLSCAVSGTSVSSNAMGWYRQAPGKQREWVG
FIDRIATTTIATSVKGRFAITRDNAKNTVYLQMSGLKPEDTAVYYCNHP
LTARWGQGTQVTVSS,
(SEQ ID NO: 97)
QVQLVESGGGLVQAGGSLRLSCAASGRTFSSYAMGWFRQAPGKEREFVA
AITWNGGTTYYADSVKGRFTISRDNAKNTVYLQMNSLKPEDTAVYYCAA
DPFNQGYWGQGTQVTVSS,
(SEQ ID NO: 98)
EVQLVESGGGLVQAGGSLRLSCAVSGSSVSSDAMGWYRQAPGNQRAWVA
FISGGGTTTYADSVKGRFTISRDNTKNTVYLHMNSLKPEDTAVYYCNHP
LTSRWGQGTQVTVSS,
(SEQ ID NO: 99)
EVQVVESGGGLVQAGGSLRLACVASRSIGSINVMGWYRQAPGKQRDLVA
RITGGGSTHYAESVKGRFTISRDNAKNTVYLQMNSLEPEDTAVYYCASM
VNPIITAWGTIGVREIPDYDYWGQGTQVTVSS,
(SEQ ID NO: 100)
QVQLVESGGGLVQAGGSLRLSCAVSGRTFSTYRMGWFRQAPGKERSFVA
AISWSGGSTTYADPVKGRFTISRDNAKNTVYLRMNSLKPEDTAVYYCND
QRGYWGQGTLVTVSS,
(SEQ ID NO: 101)
EVQVVESGGGLVQAGGSLRLSCAASGFTFTRYAMGWFRQAPGKERSFVA
AISWSGSSAGYGDSVKGRFTISRDNAKNTLYLQMNSLKPEDTAVYYCAA
DPFNQGYWGQGTQVTVSS,
(SEQ ID NO: 102)
EVQVVESGGGLVQAGGSLRLSCAASGRTFTTYRMGWFRQAPGKEREFVA
AIRWSGGRTLYADSVKGRFTISRDNAKNTAYLQMNNLRPEDTAVYYCAA
DLAEYSGTYSSPADSPAGYDYWGQGTQVTVSS,
or
(SEQ ID NO: 103)
QVQLVETGGGLVQAGDSLRLSCAASGRTLSFNTYAMGWFRQAPGKEREF
VASITWNGGSTSYADSVKGRFTITRDNAKNTATLRMNSLQPDDTAVYYC
AAARYYVSGTYFPANYWGQGTQVTVSS.

61. The therapeutic molecule of any one of claims 37 to 60, wherein the VHH domain is comprised of a sequence having at least 75%, 80%, 85%, 90%, 95%, 96%, 97%, 98%, 99%, or 100% sequence identity with the sequence of
(SEQ ID NO: 93)
QVQLVESGGGLVQAGGSLKLACAAPGLTFSSYRMGWFRQAPGQEREFVA
AIDWNGRGTYYRYYADSVKGRSTISRDNAKNTMYLQMNSLKPEDTAVYY
CAAGSIDLNWYGGMDYWGQGTQVTVSS,
(SEQ ID NO: 94)
EVQVVESGGGLVQAGGSLKLACAAPGLTFSSYRMGWFRQAPGQEREFVA
AIDWNGRGTYYRYYADSVKGRSTISRDNAKNTVYLQMNSLKPEDTAVYY
CAATTVLTDPRVLNEYATWGQGTQVTVSS,
(SEQ ID NO: 95)
QLQLVESGGGLVQPGGSLRLSCAASGSIFSINVMGWYRQAPGKQRELVA
RINGGGITHYAESVKGRFTISRDNAKNTVYLQMNSLKPEDTAAYYCKAD
VFGSSGYVETYWGQGTQVTVSS,
(SEQ ID NO: 96)
EVQVVESGGGLVQAGGSLRLSCAVSGTSVSSNAMGWYRQAPGKQREWVG
FIDRIATTTIATSVKGRFAITRDNAKNTVYLQMSGLKPEDTAVYYCNHP
LTARWGQGTQVTVSS,
(SEQ ID NO: 97)
QVQLVESGGGLVQAGGSLRLSCAASGRTFSSYAMGWFRQAPGKEREFVA
AITWNGGTTYYADSVKGRFTISRDNAKNTVYLQMNSLKPEDTAVYYCAA
DPFNQGYWGQGTQVTVSS,
(SEQ ID NO: 98)
EVQLVESGGGLVQAGGSLRLSCAVSGSSVSSDAMGWYRQAPGNQRAWVA
FISGGGTTTYADSVKGRFTISRDNTKNTVYLHMNSLKPEDTAVYYCNHP
LTSRWGQGTQVTVSS,
(SEQ ID NO: 99)
EVQVVESGGGLVQAGGSLRLACVASRSIGSINVMGWYRQAPGKQRDLVA
RITGGGSTHYAESVKGRFTISRDNAKNTVYLQMNSLEPEDTAVYYCASM
VNPIITAWGTIGVREIPDYDYWGQGTQVTVSS,
(SEQ ID NO: 100)
QVQLVESGGGLVQAGGSLRLSCAVSGRTFSTYRMGWFRQAPGKERSFVA
AISWSGGSTTYADPVKGRFTISRDNAKNTVYLRMNSLKPEDTAVYYCND
QRGYWGQGTLVTVSS,
(SEQ ID NO: 101)
EVQVVESGGGLVQAGGSLRLSCAASGFTFTRYAMGWFRQAPGKERSFVA
AISWSGSSAGYGDSVKGRFTISRDNAKNTLYLQMNSLKPEDTAVYYCAA
DPFNQGYWGQGTQVTVSS,
(SEQ ID NO: 102)
EVQVVESGGGLVQAGGSLRLSCAASGRTFTTYRMGWFRQAPGKEREFVA
AIRWSGGRTLYADSVKGRFTISRDNAKNTAYLQMNNLRPEDTAVYYCAA
DLAEYSGTYSSPADSPAGYDYWGQGTQVTVSS,
or
(SEQ ID NO: 103)
QVQLVETGGGLVQAGDSLRLSCAASGRTLSFNTYAMGWFRQAPGKEREF
VASITWNGGSTSYADSVKGRFTITRDNAKNTATLRMNSLQPDDTAVYYC
AAARYYVSGTYFPANYWGQGTQVTVSS.

62. The therapeutic molecule of any one of claims 37 to 61, wherein the agent is an antibody or fragment thereof, a peptide, a vaccine, a small molecule, a polynucleotide, a radioisotope, a toxin, an enzyme, an anticoagulant, a hormone, a cytokine, an anti-inflammatory molecule, an RNAi, an antibiotic, or an antibody-antibiotic conjugate.

63. The therapeutic molecule of claim 62, wherein the agent is an antibiotic, an antibody or fragment thereof, a peptide or a vaccine.

64. The therapeutic molecule of claim 62 or claim 63, wherein the VHH domain is genetically fused or chemically conjugated to the agent.

65. The therapeutic molecule of claim 64, further comprising a linker between the VHH domain and the agent.

66. The therapeutic molecule of claim 65, wherein the linker is a polypeptide.

67. The therapeutic molecule of claim 66, wherein the linker is a flexible linker comprising a sequence selected from the group consisting of EPKTPKPQPQPQLQPQPNPTTESKSPK (SEQ ID NO: 130), (EAAAK)n (SEQ ID NO: 147), (GGGGS)n (SEQ ID NO: 148) and (GGGS)n (SEQ ID NO: 149), wherein n is an integer from 1 to 20.

68. The therapeutic molecule of any one of claims 37 to 67, wherein the VHH domain is chemically-conjugated to the agent.

69. The therapeutic molecule of any one of claims 37 to 67, wherein the VHH domain is non-covalently bound to the agent.

70. A pharmaceutical composition comprising the therapeutic molecule of any of claims 37 to 69 and a pharmaceutically acceptable carrier.

71. A method of delivering a therapeutic molecule to a mucosal lumen of a subject, the method comprising administering to the subject the therapeutic molecule of any of claims 37 to 69.

72. The method of claim 71, wherein the therapeutic molecule is delivered to the mucosal lumen via forward transcytosis from the basolateral surface of a mucosal epithelial cell to the apical surface of the mucosal epithelial cell.

73. The method of claim 71, wherein the mucosal epithelial cell is at or adjacent to the mucosal lumen.

74. The method of claim 72 or 73, wherein the mucosal lumen is in the lung or in the gastrointestinal tract of the subject.

75. The method of any one of claims 72 to 73, wherein the mucosal epithelial cell is a cancer cell.

76. The method of claim 75, wherein the cancer cell is a lung cancer cell, an esophageal cancer cell, a stomach cancer cell, a duodenal cancer cell, a liver cancer cell, a bladder cancer cell, a sinus cancer cell, a nasal cavity cancer cell, an endometrial cancer cell or a colorectal cancer cell.

77. The method of any one of claims 72 to 76, wherein the cell is in a subject.

78. A method of delivering a therapeutic molecule to an organ of a subject, the method comprising administering to the subject the therapeutic molecule of any of claims 37 to 67.

79. The method of claim 78, wherein the organ is selected from the group consisting of gastrointestinal track, small intestine, large intestine, stomach, esophagus, salivary gland, lung, vagina, uterus, and lacrimal gland.

80. The method of claim 79, wherein the organ is lung.

81. The method of any one of claims 71 to 80, wherein the agent is an antibody or fragment thereof, a peptide, a vaccine, a small molecule, a polynucleotide, a molecule comprising a radioactive isotope, a toxin, an enzyme, an anticoagulant, a hormone, a cytokine, an anti-inflammatory molecule, an RNAi, an mRNA, a self-replicating RNA, an antibiotic, or an antibody-antibiotic conjugate.

82. The method of claim 81, wherein the agent is an antibiotic, an antibody or fragment thereof, a peptide or a vaccine.

83. The method of claim 82, wherein the antibiotic is selected from the group consisting of a macrolide antibiotic, a fluoroquinolone, a tetracycline, amoxicillin, ceftriaxone, penicillin G, linezolid, moxifloxacin, and azithromycin.

84. The method of any one of claims 71 to 83, wherein the therapeutic molecule is administered to the bloodstream of the subject.

85. The method of any one of claims 71 to 83, wherein the molecule is administered intravenously or subcutaneously.

86. A method of delivering a therapeutic molecule into systemic circulation in a subject, the method comprising administering to the subject the therapeutic molecule of any of claims 37 to 69.

87. The method of claim 86, wherein the therapeutic molecule is delivered into the systemic circulation via reverse transcytosis from the apical surface of an epithelial cell to the basolateral surface of the epithelial cell.

88. The method of claim 86, wherein the therapeutic molecule is delivered by oral delivery, buccal delivery, nasal delivery or inhalation delivery.

89. The method of any one of claims 86 to 88, wherein the agent is a peptide, an antibody or fragment thereof or a vaccine.

90. A method of delivering a therapeutic molecule into Lamina propria of a subject, the method comprising administering to the subject the therapeutic molecule of any of claims 37 to 69.

91. The method of claim 90, wherein the therapeutic molecule is delivered into the Lamina propria via reverse transcytosis from the apical surface of an epithelial cell to the basolateral surface of the epithelial cell.

92. The method of claim 90, wherein the therapeutic molecule is delivered by oral delivery or buccal delivery.

93. The method of any one of claims 90 to 92, wherein the agent is a peptide or an antibody or fragment thereof.

94. A method of increasing the rate of pIgR-mediated transcytosis across an epithelial cell comprising contacting the cell with (i) a VHH domain that binds to an extracellular domain 1, an extracellular domain 2, an extracellular domain 1-2, an extracellular domain 3, an extracellular domain 2-3, an extracellular domain 4-5, or an extracellular domain 5 of polymeric immunoglobulin receptor (pIgR) or (ii) a therapeutic molecule comprising an agent and the VHH domain.

95. The method of claim 94, wherein the transcytosis is forward transcytosis.

96. The method of claim 94, wherein the transcytosis is reverse transcytosis.

97. A method of modulating a function of pIgR in a cell comprising contacting the cell with (i) a VHH domain that binds to an extracellular domain 1, an extracellular domain 2, an extracellular domain 1-2, an extracellular domain 3, an extracellular domain 2-3, an extracellular domain 4-5, or an extracellular domain 5 of polymeric immunoglobulin receptor (pIgR) or (ii) a therapeutic molecule comprising an agent and the VHH domain.

98. The method of claim 97, wherein the modulating the function of pIgR in the cell is activating said function of pIgR in said cell.

99. The method of claim 97, wherein the modulating the function of pIgR in the cell is inhibiting said function of pIgR in said cell.

100. A method of delivery to a pIgR-expressing cell comprising contacting the cell with a VHH domain or a therapeutic molecule, wherein the VHH domain binds to an extracellular domain 1, an extracellular domain 2, an extracellular domain 1-2, an extracellular domain 3, an extracellular domain 2-3, an extracellular domain 4-5, or an extracellular domain 5 of polymeric immunoglobulin receptor (pIgR), and wherein the therapeutic molecule comprises an agent and the VHH domain.

101. The method of claim 98, wherein the method of delivery is oral delivery, buccal delivery, nasal delivery or inhalation delivery.

102. The method of any one of claims 94 to 100, wherein the VHH domain competes with IgA binding to the pIgR.

103. The method of any one of claims 94 to 100, wherein the VHH domain promotes IgA binding to the pIgR.

104. The method of any one of claims 94 to 103, wherein the K_{D} of the binding of the VHH domain to pIgR is from about 4 to about 525 nM.

105. The method of any one of claims 94 to 104, wherein the K_{D} of the binding of the VHH domain to pIgR is less than about 50 nM.

106. The method of any one of claims 94 to 105, wherein the K_{D} of the binding of the VHH domain to pIgR is from about 4 to about 34 nM.

107. The method of any one of claims 94 to 106, wherein the Tₘ of the VHH domain is from about 53 to about 77° C.

108. The method of any one of claims 94 to 107, wherein the Tₘ of the VHH domain is from 53.9 to 76.4° C.

109. The method of any one of claims 94 to 108, wherein the VHH domain binds to the extracellular domain 1 of pIgR.

110. The method of any one of claims 94 to 108, wherein the VHH domain binds to the extracellular domain 2 of pIgR.

111. The method of any one of claims 94 to 108, wherein the VHH domain binds to the extracellular domain 1-2 of pIgR.

112. The method of any one of claims 94 to 108, wherein the VHH domain binds to the extracellular domain 3 of pIgR.

113. The method of any one of claims 94 to 108, wherein the VHH domain binds to the extracellular domain 2-3 of pIgR.

114. The method of any one of claims 94 to 108, wherein the VHH domain binds to the extracellular domain 4-5 of pIgR.

115. The method of any one of claims 94 to 108, wherein the VHH domain binds to the extracellular domain 5 of pIgR.

116. The method of any one of claims 94 to 115, wherein pIgR is human pIgR.

117. The method of any one of claims 94 to 115, wherein pIgR is mouse pIgR.

118. The method of any one of claims 94 to 115, wherein the VHH domain does not detectably bind to the amino acid sequence of EKAVADTRDQADGSRASVDSGSSEEQGGSSR (SEQ ID NO: 143), EREIQNVGDQAQENRASGDAGSADGQSRSSSSK (SEQ ID NO: 144), or EREIQNVRDQAQENRASGDAGSADGQSRSSSSK (SEQ ID NO: 145).

119. The method of any one of claims 94 to 118, wherein the VHH domain comprises a CDR3 sequence of GSIDLNWYGGMDY (SEQ ID NO: 60), TTVLTDPRVLNEYAT (SEQ ID NO: 61), DVFGSSGYVETY (SEQ ID NO: 62), PLTAR (SEQ ID NO: 63), DPFNQGY (SEQ ID NO: 64), PLTSR (SEQ ID NO: 65), MVNPIITAWGTIGVREIPDYDY (SEQ ID NO: 66), DQRGY (SEQ ID NO: 67), QRGY (SEQ ID NO: 271), DPFNQGY (SEQ ID NO: 68), DLAEYSGTYSSPADSPAGYDY (SEQ ID NO: 69), ARYYVSGTYFPANY (SEQ ID NO: 70), GSIDLNWYGGMDY (SEQ ID NO: 71), SIDLNWYGGMD (SEQ ID NO: 272), TTVLTDPRVLNEYAT (SEQ ID NO: 72), TVLTDPRVLNEYA (SEQ ID NO: 273), DVFGSSGYVETY (SEQ ID NO: 73), VFGSSGYVET (SEQ ID NO: 274), PLTAR (SEQ ID NO: 74), LTA (SEQ ID NO: 275), DPFNQGY (SEQ ID NO: 75), PFNQG (SEQ ID NO: 276), PLTSR (SEQ ID NO: 76), LTS (SEQ ID NO: 277), MVNPIITAWGTIGVREIPDYDY (SEQ ID NO: 77), VNPIITAWGTIGVREIPDYD (SEQ ID NO: 278), DQRGY (SEQ ID NO: 78), RG (SEQ ID NO: 279), DPFNQGY (SEQ ID NO: 79), PFNQG (SEQ ID NO: 280), DLAEYSGTYSSPADSPAGYDY (SEQ ID NO: 80), LAEYSGTYSSPADSPAGYD (SEQ ID NO: 281), ARYYVSGTYFPANY (SEQ ID NO: 81), RYYVSGTYFPAN (SEQ ID NO: 282), CAAGSIDLNWYGGMDY (SEQ ID NO: 82), AAGSIDLNWYGGMDY (SEQ ID NO: 283), CAATTVLTDPRVLNEYAT (SEQ ID NO: 83), AATTVLTDPRVLNEYAT (SEQ ID NO: 284), KADVFGSSGYVETY (SEQ ID NO: 84), NHPLTAR (SEQ ID NO: 85), AADPFNQGY (SEQ ID NO: 86), NHPLTSR (SEQ ID NO: 87), ASMVNPIITAWGTIGVREIPDYDY (SEQ ID NO: 88), NDQRGY (SEQ ID NO: 89), AADPFNQGY (SEQ ID NO: 90), AADLAEYSGTYSSPADSPAGYDY (SEQ ID NO: 91), AAARYYVSGTYFPANY (SEQ ID NO: 92), GSIDLNWYGGMDY (SEQ ID NO: 214), TTVLTDPRVLNEYAT (SEQ ID NO: 215), DVFGSSGYVETY (SEQ ID NO: 216), PLTAR (SEQ ID NO: 217), DPFNQGY (SEQ ID NO: 218), PLTSR (SEQ ID NO: 219), MVNPIITAWGTIGVREIPDYDY (SEQ ID NO: 220), QRGY (SEQ ID NO: 221), DPFNQGY (SEQ ID NO: 222), DLAEYSGTYSSPADSPAGYDY (SEQ ID NO: 223), ARYYVSGTYFPANY (SEQ ID NO: 224), AAGSIDLNWYGGMD (SEQ ID NO: 225), AATTVLTDPRVLNEYA (SEQ ID NO: 226), KADVFGSSGYVET (SEQ ID NO: 227), NHPLTA (SEQ ID NO: 228), AADPFNQG (SEQ ID NO: 229), NHPLTS (SEQ ID NO: 230), ASMVNPIITAWGTIGVREIPDYD (SEQ ID NO: 231), NDQRG (SEQ ID NO: 232), AADPFNQG (SEQ ID NO: 233), AADLAEYSGTYSSPADSPAGYD (SEQ ID NO: 234), AAARYYVSGTYFPAN (SEQ ID NO: 235), GSIDLNWYGGMDY (SEQ ID NO: 236), TTVLTDPRVLNEYAT (SEQ ID NO: 237), DVFGSSGYVETY (SEQ ID NO: 238), PLTAR (SEQ ID NO: 239), DPFNQGY (SEQ ID NO: 240), PLTSR (SEQ ID NO: 241), MVNPIITAWGTIGVREIPDYDY (SEQ ID NO: 242), QRGY (SEQ ID NO: 243), DPFNQGY (SEQ ID NO: 244), DLAEYSGTYSSPADSPAGYDY (SEQ ID NO: 245), or ARYYVSGTYFPANY (SEQ ID NO: 246).

120. The method of any one of claims 94 to 119, wherein the VHH domain comprises a CDR2 sequence of AIDWNGRGTYYRYYADSVKG (SEQ ID NO: 30), RINGGGITHYAESVKG (SEQ ID NO: 31), FIDRIATTTIATSVKG (SEQ ID NO: 32), AITWNGGTTYYADSVKG (SEQ ID NO: 33), FISGGGTTTYADSVKG (SEQ ID NO: 34), RITGGGSTHYAESVKG (SEQ ID NO: 35), AISWSGGSTTYADPVKG (SEQ ID NO: 36), AISWSGSSAGYGDSVKG (SEQ ID NO: 37), AIRWSGGRTLYADSVKG (SEQ ID NO: 38), SITWNGGSTSYADSVKG (SEQ ID NO: 39), DWNGRGTYY (SEQ ID NO: 40), WNGRGTY (SEQ ID NO: 260), NGGGI (SEQ ID NO: 41), GGG (SEQ ID NO: 261), DRIAT (SEQ ID NO: 42), RIA (SEQ ID NO: 262), TWNGGT (SEQ ID NO: 43), WNGG (SEQ ID NO: 263), SGGGT (SEQ ID NO: 44), GGG (SEQ ID NO: 264), TGGGS (SEQ ID NO: 45), GGG (SEQ ID NO: 265), SWSGGS (SEQ ID NO: 46), WSGG (SEQ ID NO: 266), SWSGSS (SEQ ID NO: 47), WSGS (SEQ ID NO: 267), RWSGGR (SEQ ID NO: 48), WSGG (SEQ ID NO: 268), TWNGGS (SEQ ID NO: 49), WNGG (SEQ ID NO: 269), IDWNGRGTYY (SEQ ID NO: 50), IDWNGRGTYYR (SEQ ID NO: 270), INGGGIT (SEQ ID NO: 51), IDRIATT (SEQ ID NO: 52), ITWNGGTT (SEQ ID NO: 53), ISGGGTT (SEQ ID NO: 54), ITGGGST (SEQ ID NO: 55), ISWSGGST (SEQ ID NO: 56), ISWSGSSA (SEQ ID NO: 57), IRWSGGRT (SEQ ID NO: 58), ITWNGGST (SEQ ID NO: 59), AIDWNGRGTYYRYYADSVKG (SEQ ID NO: 184), RINGGGITHYAESVKG (SEQ ID NO: 185), FIDRIATTTIATSVKG (SEQ ID NO: 186), AITWNGGTTYYADSVKG (SEQ ID NO: 187), FISGGGTTTYADSVKG (SEQ ID NO: 188), RITGGGSTHYAESVKG (SEQ ID NO: 189), AISWSGGSTTYADPVKG (SEQ ID NO: 190), AISWSGSSAGYGDSVKG (SEQ ID NO: 191), AIRWSGGRTLYADSVKG (SEQ ID NO: 192), SITWNGGSTSYADSVKG (SEQ ID NO: 193), FVAAIDWNGRGTYYRY (SEQ ID NO: 194), LVARINGGGITH (SEQ ID NO: 195), WVGFIDRIATTT (SEQ ID NO: 196), FVAAITWNGGTTY (SEQ ID NO: 197), WVAFISGGGTTT (SEQ ID NO: 198), LVARITGGGSTH (SEQ ID NO: 199), FVAAISWSGGSTT (SEQ ID NO: 200), FVAAISWSGSSAG (SEQ ID NO: 201), FVAAIRWSGGRTL (SEQ ID NO: 202), FVASITWNGGSTS (SEQ ID NO: 203), AIDWNGRGTYYRY (SEQ ID NO: 204), RINGGGITH (SEQ ID NO: 205), FIDRIATTT (SEQ ID NO: 206), AITWNGGTTY (SEQ ID NO: 207), FISGGGTTT (SEQ ID NO: 208), RITGGGSTH (SEQ ID NO: 209), AISWSGGSTT (SEQ ID NO: 210), AISWSGSSAG (SEQ ID NO: 211), AIRWSGGRTL (SEQ ID NO: 212), or SITWNGGSTS (SEQ ID NO: 213).

121. The method of any one of claims 94 to 120, wherein the VHH domain comprises a CDR1 sequence of SYRMG (SEQ ID NO: 1), INVMG (SEQ ID NO: 2), SNAMG (SEQ ID NO: 3), SYAMG (SEQ ID NO: 4), SDAMG (SEQ ID NO: 5), INVMG (SEQ ID NO: 6), TYRMG (SEQ ID NO: 7), RYAMG (SEQ ID NO: 8), FTTYRMG (SEQ ID NO: 258), TYRMG (SEQ ID NO: 259), FNTYAMG (SEQ ID NO: 9), GLTFSSY (SEQ ID NO: 10), GSIFSIN (SEQ ID NO: 11), GTSVSSN (SEQ ID NO: 12), GRTFSSY (SEQ ID NO: 13), GSSVSSD (SEQ ID NO: 14), RSIGSIN (SEQ ID NO: 15), GRTFSTY (SEQ ID NO: 16), GFTFTRY (SEQ ID NO: 17), GRTFTTY (SEQ ID NO: 18), GRTLSFNTY (SEQ ID NO: 19), GLTFSSYR (SEQ ID NO: 20), GSIFSINV (SEQ ID NO: 21), GTSVSSNA (SEQ ID NO: 22), GRTFSSYA (SEQ ID NO: 23), GSSVSSDA (SEQ ID NO: 24), RSIGSINV (SEQ ID NO: 25), GRTFSTYR (SEQ ID NO: 26), GFTFTRYA (SEQ ID NO: 27), GRTFTTYR (SEQ ID NO: 28), GRTLSFNTYA (SEQ ID NO: 29), GLTFSSYRMG (SEQ ID NO: 154), GSIFSINVMG (SEQ ID NO: 155), GTSVSSNAMG (SEQ ID NO: 156), GRTFSSYAMG (SEQ ID NO: 157), GSSVSSDAMG (SEQ ID NO: 158), RSIGSINVMG (SEQ ID NO: 159), GRTFSTYRMG (SEQ ID NO: 160), GFTFTRYAMG (SEQ ID NO: 161), GRTFTTYRMG (SEQ ID NO: 162), GRTLSFNTYAMG (SEQ ID NO: 163), SSYRMG (SEQ ID NO: 164), SINVMG (SEQ ID NO: 165), SSNAMG (SEQ ID NO: 166), SSYAMG (SEQ ID NO: 167), SSDAMG (SEQ ID NO: 168), SINVMG (SEQ ID NO: 169), STYRMG (SEQ ID NO: 170), TRYAMG (SEQ ID NO: 171), TTYRMG (SEQ ID NO: 172), SFNTYAMG (SEQ ID NO: 173), GLTFSSYRMG (SEQ ID NO: 174), GSIFSINVMG (SEQ ID NO: 175), GTSVSSNAMG (SEQ ID NO: 176), GRTFSSYAMG (SEQ ID NO: 177), GSSVSSDAMG (SEQ ID NO: 178), RSIGSINVMG (SEQ ID NO: 179), GRTFSTYRMG (SEQ ID NO: 180), GFTFTRYAMG (SEQ ID NO: 181), GRTFTTYRMG (SEQ ID NO: 182), or GRTLSFNTYAMG (SEQ ID NO: 183).

122. The method of any one of claims 94 to 121, wherein the VHH domain comprises a CDR1 sequence, a CDR2 sequence, and a CDR3 sequence of the VHH domain selected from the group consisting of:
a) VHH1:
i) the CDR1 sequence of SYRMG (SEQ ID NO: 1), the CDR2 sequence of AIDWNGRGTYYRYYADSVKG (SEQ ID NO: 30), and the CDR3 sequence of GSIDLNWYGGMDY (SEQ ID NO: 60);
ii) the CDR1 sequence of GLTFSSY (SEQ ID NO: 10), the CDR2 sequence of DWNGRGTYY (SEQ ID NO: 40) or WNGRGTY (SEQ ID NO: 260), and the CDR3 sequence of GSIDLNWYGGMDY (SEQ ID NO: 71) or SIDLNWYGGMD (SEQ ID NO: 272);
iii) the CDR1 sequence of GLTFSSYR (SEQ ID NO: 20), the CDR2 sequence of IDWNGRGTYY (SEQ ID NO: 50) or IDWNGRGTYYR (SEQ ID NO: 270), and the CDR3 sequence of CAAGSIDLNWYGGMDY (SEQ ID NO: 82) or AAGSIDLNWYGGMDY (SEQ ID NO: 283);
iv) the CDR1 sequence of GLTFSSYRMG (SEQ ID NO: 154), the CDR 2 sequence of AIDWNGRGTYYRYYADSVKG (SEQ ID NO: 184), and the CDR3 sequence of GSIDLNWYGGMDY (SEQ ID NO: 214);
v) the CDR1 sequence of SSYRMG (SEQ ID NO: 164), the CDR2 sequence of FVAAIDWNGRGTYYRY (SEQ ID NO: 194), and the CDR3 sequence of AAGSIDLNWYGGMD (SEQ ID NO: 225); or
vi) the CDR1 sequence of GLTFSSYRMG (SEQ ID NO: 174), the CDR2 sequence of AIDWNGRGTYYRY (SEQ ID NO: 204), and the CDR3 sequence of GSIDLNWYGGMDY (SEQ ID NO: 236);
b) VHH2:
i) the CDR1 sequence of SYRMG (SEQ ID NO: 1), the CDR2 sequence of AIDWNGRGTYYRYYADSVKG (SEQ ID NO: 30), and the CDR3 sequence of TTVLTDPRVLNEYAT (SEQ ID NO: 61);
ii) the CDR1 sequence of GLTFSSY (SEQ ID NO: 10), the CDR2 sequence of DWNGRGTYY (SEQ ID NO: 40) or WNGRGTY (SEQ ID NO: 260), and the CDR3 sequence of TTVLTDPRVLNEYAT (SEQ ID NO: 72) or TVLTDPRVLNEYA (SEQ ID NO: 273);
iii) the CDR1 sequence of GLTFSSYR (SEQ ID NO: 20), the CDR2 sequence of IDWNGRGTYY (SEQ ID NO: 50) or IDWNGRGTYYR (SEQ ID NO: 270), and the CDR3 sequence of CAATTVLTDPRVLNEYAT (SEQ ID NO: 83) or AATTVLTDPRVLNEYAT (SEQ ID NO: 284);
iv) the CDR1 sequence of GLTFSSYRMG (SEQ ID NO: 154), the CDR2 sequence of AIDWNGRGTYYRYYADSVKG (SEQ ID NO: 184), and the CDR3 sequence of TTVLTDPRVLNEYAT (SEQ ID NO: 215);
v) the CDR1 sequence of SSYRMG (SEQ ID NO: 164), the CDR2 sequence of FVAAIDWNGRGTYYRY (SEQ ID NO: 194), and the CDR3 sequence of AATTVLTDPRVLNEYA (SEQ ID NO: 226); or
vi) the CDR1 sequence of GLTFSSYRMG (SEQ ID NO: 174), the CDR2 sequence of AIDWNGRGTYYRY (SEQ ID NO: 204), and the CDR3 sequence of TTVLTDPRVLNEYAT (SEQ ID NO: 237);
c) VHH3:
i) the CDR1 sequence of INVMG (SEQ ID NO: 2), the CDR2 sequence of RINGGGITHYAESVKG (SEQ ID NO: 31), and the CDR3 sequence of DVFGSSGYVETY (SEQ ID NO: 62);
ii) the CDR1 sequence of GSIFSIN (SEQ ID NO: 11), the CDR2 sequence of NGGGI (SEQ ID NO: 41) or GGG (SEQ ID NO: 261), and the CDR3 sequence of DVFGSSGYVETY (SEQ ID NO: 73) or VFGSSGYVET (SEQ ID NO: 274);
iii) the CDR1 sequence of GSIFSINV (SEQ ID NO: 21), the CDR2 sequence of INGGGIT (SEQ ID NO: 51), and the CDR3 sequence of KADVFGSSGYVETY (SEQ ID NO: 84);
iv) the CDR1 sequence of GSIFSINVMG (SEQ ID NO: 155), the CDR2 sequence of RINGGGITHYAESVKG (SEQ ID NO: 185), and the CDR3 sequence of DVFGSSGYVETY (SEQ ID NO: 216);
v) the CDR1 sequence of SINVMG (SEQ ID NO: 165), the CDR2 sequence of LVARINGGGITH (SEQ ID NO: 195), and the CDR3 sequence of KADVFGSSGYVET (SEQ ID NO: 227); or
vi) the CDR1 sequence of GSIFSINVMG (SEQ ID NO: 175), the CDR2 sequence of RINGGGITH (SEQ ID NO: 205), and the CDR3 sequence of DVFGSSGYVETY (SEQ ID NO: 238);
d) VHH4:
i) the CDR1 sequence of SNAMG (SEQ ID NO: 3), the CDR2 sequence of FIDRIATTTIATSVKG (SEQ ID NO: 32), and the CDR3 sequence of PLTAR (SEQ ID NO: 63);
ii) the CDR1 sequence of GTSVSSN (SEQ ID NO: 12), the CDR2 sequence of DRIAT (SEQ ID NO: 42) or RIA (SEQ ID NO: 262), and the CDR3 sequence of PLTAR (SEQ ID NO: 74) or LTA (SEQ ID NO: 275);
iii) the CDR1 sequence of GTSVSSNA (SEQ ID NO: 22), the CDR2 sequence of IDRIATT (SEQ ID NO: 52), and the CDR3 sequence of NHPLTAR (SEQ ID NO: 85);
iv) the CDR1 sequence of GTSVSSNAMG (SEQ ID NO: 156), the CDR2 sequence of FIDRIATTTIATSVKG (SEQ ID NO: 186), and the CDR3 sequence of PLTAR (SEQ ID NO: 217);
v) the CDR1 sequence of SSNAMG (SEQ ID NO: 166), the CDR2 sequence of WVGFIDRIATTT (SEQ ID NO: 196), and the CDR3 sequence of NHPLTA (SEQ ID NO: 228); or
vi) the CDR1 sequence of GTSVSSNAMG (SEQ ID NO: 176), the CDR2 sequence of FIDRIATTT (SEQ ID NO: 206), and the CDR3 sequence of PLTAR (SEQ ID NO: 239);
e) VHH5:
i) the CDR1 sequence of SYAMG (SEQ ID NO: 4), the CDR2 sequence of AITWNGGTTYYADSVKG (SEQ ID NO: 33), and the CDR3 sequence of DPFNQGY (SEQ ID NO: 64);
ii) the CDR1 sequence of GRTFSSY (SEQ ID NO: 13), the CDR2 sequence of TWNGGT (SEQ ID NO: 43) or WNGG (SEQ ID NO: 263), and the CDR3 sequence of DPFNQGY (SEQ ID NO: 75) or PFNQG (SEQ ID NO: 276);
iii) the CDR1 sequence of GRTFSSYA (SEQ ID NO: 23), the CDR2 sequence of ITWNGGTT (SEQ ID NO: 53), and the CDR3 sequence of AADPFNQGY (SEQ ID NO: 86);
iv) the CDR1 sequence of GRTFSSYAMG (SEQ ID NO: 157), the CDR2 sequence of AITWNGGTTYYADSVKG (SEQ ID NO: 187), and the CDR3 sequence of DPFNQGY (SEQ ID NO: 218);
v) the CDR1 sequence of SSYAMG (SEQ ID NO: 167), the CDR2 sequence of FVAAITWNGGTTY (SEQ ID NO: 197), and the CDR3 sequence of AADPFNQG (SEQ ID NO: 229); or
vi) the CDR1 sequence of GRTFSSYAMG (SEQ ID NO: 177), the CDR2 sequence of AITWNGGTTY (SEQ ID NO: 207), and the CDR3 sequence of DPFNQGY (SEQ ID NO: 240);
f) VHH6:
i) the CDR1 sequence of SDAMG (SEQ ID NO: 5), the CDR2 sequence of FISGGGTTTYADSVKG (SEQ ID NO: 34), and the CDR3 sequence of PLTSR (SEQ ID NO: 65);
ii) the CDR1 sequence of GSSVSSD (SEQ ID NO: 14), the CDR2 sequence of SGGGT (SEQ ID NO: 44) or GGG (SEQ ID NO: 264), and the CDR3 sequence of PLTSR (SEQ ID NO: 76) or LTS (SEQ ID NO: 277);
iii) the CDR1 sequence of GSSVSSDA (SEQ ID NO: 24), the CDR2 sequence of ISGGGTT (SEQ ID NO: 54), and the CDR3 sequence of NHPLTSR (SEQ ID NO: 87);
iv) the CDR1 sequence of GSSVSSDAMG (SEQ ID NO: 158), the CDR2 sequence of FISGGGTTTYADSVKG (SEQ ID NO: 188), and the CDR3 sequence of PLTSR (SEQ ID NO: 219);
v) the CDR1 sequence of SSDAMG (SEQ ID NO: 168), the CDR2 sequence of WVAFISGGGTTT (SEQ ID NO: 198), and the CDR3 sequence of NHPLTS (SEQ ID NO: 230); or
vi) the CDR1 sequence of GSSVSSDAMG (SEQ ID NO: 178), the CDR2 sequence of FISGGGTTT (SEQ ID NO: 208), and the CDR3 sequence of PLTSR (SEQ ID NO: 241);
g) VHH7:
i) the CDR1 sequence of INVMG (SEQ ID NO: 6), the CDR2 sequence of RITGGGSTHYAESVKG (SEQ ID NO: 35), and the CDR3 sequence of MVNPIITAWGTIGVREIPDYDY (SEQ ID NO: 66);
ii) the CDR1 sequence of RSIGSIN (SEQ ID NO: 15), the CDR2 sequence of TGGGS (SEQ ID NO: 45) or GGG (SEQ ID NO: 265), and the CDR3 sequence of MVNPIITAWGTIGVREIPDYDY (SEQ ID NO: 77) or VNPIITAWGTIGVREIPDYD (SEQ ID NO: 278);
iii) the CDR1 sequence of RSIGSINV (SEQ ID NO: 25), the CDR2 sequence of ITGGGST (SEQ ID NO: 55), and the CDR3 sequence of ASMVNPIITAWGTIGVREIPDYDY (SEQ ID NO: 88);
iv) the CDR1 sequence of RSIGSINVMG (SEQ ID NO: 159), the CDR2 sequence of RITGGGSTHYAESVKG (SEQ ID NO: 189), and the CDR3 sequence of MVNPIITAWGTIGVREIPDYDY (SEQ ID NO: 220);
v) the CDR1 sequence of SINVMG (SEQ ID NO: 169), the CDR2 sequence of LVARITGGGSTH (SEQ ID NO: 199), and the CDR3 sequence of ASMVNPIITAWGTIGVREIPDYD (SEQ ID NO: 231); or
vi) the CDR1 sequence of RSIGSINVMG (SEQ ID NO: 179), the CDR2 sequence of RITGGGSTH (SEQ ID NO: 209), and the CDR3 sequence of MVNPIITAWGTIGVREIPDYDY (SEQ ID NO: 242);
h) VHH9:
i) the CDR1 sequence of TYRMG (SEQ ID NO: 7), the CDR2 sequence of AISWSGGSTTYADPVKG (SEQ ID NO: 36), and the CDR3 sequence of DQRGY (SEQ ID NO: 67) or QRGY (SEQ ID NO: 271);
ii) the CDR1 sequence of GRTFSTY (SEQ ID NO: 16), the CDR2 sequence of SWSGGS (SEQ ID NO: 46) or WSGG (SEQ ID NO: 266), and the CDR3 sequence of DQRGY (SEQ ID NO: 78) or RG (SEQ ID NO: 279);
iii) the CDR1 sequence of GRTFSTYR (SEQ ID NO: 26), the CDR2 sequence of ISWSGGST (SEQ ID NO: 56), and the CDR3 sequence of NDQRGY (SEQ ID NO: 89);
iv) the CDR1 sequence of GRTFSTYRMG (SEQ ID NO: 160), the CDR2 sequence of AISWSGGSTTYADPVKG (SEQ ID NO: 190), and the CDR3 sequence of QRGY (SEQ ID NO: 221);
v) the CDR1 sequence of STYRMG (SEQ ID NO: 170), the CDR2 sequence of FVAAISWSGGSTT (SEQ ID NO: 200), and the CDR3 sequence of NDQRG (SEQ ID NO: 232); or
vi) the CDR1 sequence of GRTFSTYRMG (SEQ ID NO: 180), the CDR2 sequence of AISWSGGSTT (SEQ ID NO: 210), and the CDR3 sequence of QRGY (SEQ ID NO: 243);
i) VHH10:
i) the CDR1 sequence of RYAMG (SEQ ID NO: 8), the CDR2 sequence of AISWSGSSAGYGDSVKG (SEQ ID NO: 37), and the CDR3 sequence of DPFNQGY (SEQ ID NO: 68);
ii) the CDR1 sequence of GFTFTRY (SEQ ID NO: 17), the CDR2 sequence of SWSGSS (SEQ ID NO: 47) or WSGS (SEQ ID NO: 267), and the CDR3 sequence of DPFNQGY (SEQ ID NO: 79) or PFNQG (SEQ ID NO: 280);
iii) the CDR1 sequence of GFTFTRYA (SEQ ID NO: 27), the CDR2 sequence of ISWSGSSA (SEQ ID NO: 57), and the CDR3 sequence of AADPFNQGY (SEQ ID NO: 90);
iv) the CDR1 sequence of GFTFTRYAMG (SEQ ID NO: 161), the CDR2 sequence of AISWSGSSAGYGDSVKG (SEQ ID NO: 191), and the CDR3 sequence of DPFNQGY (SEQ ID NO: 222);
v) the CDR1 sequence of TRYAMG (SEQ ID NO: 171), the CDR2 sequence of FVAAISWSGSSAG (SEQ ID NO: 201), and the CDR3 sequence of AADPFNQG (SEQ ID NO: 233); or
vi) the CDR1 sequence of GFTFTRYAMG (SEQ ID NO: 181), the CDR2 sequence of AISWSGSSAG (SEQ ID NO: 211), and the CDR3 sequence of DPFNQGY (SEQ ID NO: 244);
j) VHH11:
i) the CDR1 sequence of FTTYRMG (SEQ ID NO: 258) or TYRMG (SEQ ID NO: 259), the CDR2 sequence of AIRWSGGRTLYADSVKG (SEQ ID NO: 38), and the CDR3 sequence of DLAEYSGTYSSPADSPAGYDY (SEQ ID NO: 69);
ii) the CDR1 sequence of GRTFTTY (SEQ ID NO: 18), the CDR2 sequence of RWSGGR (SEQ ID NO: 48) or WSGG (SEQ ID NO: 268), and the CDR3 sequence of DLAEYSGTYSSPADSPAGYDY (SEQ ID NO: 80) or LAEYSGTYSSPADSPAGYD (SEQ ID NO: 281);
iii) the CDR1 sequence of GRTFTTYR (SEQ ID NO: 28), the CDR2 sequence of IRWSGGRT (SEQ ID NO: 58), and the CDR3 sequence of AADLAEYSGTYSSPADSPAGYDY (SEQ ID NO: 91);
iv) the CDR1 sequence of GRTFTTYRMG (SEQ ID NO: 162), the CDR2 sequence of AIRWSGGRTLYADSVKG (SEQ ID NO: 192), and the CDR3 sequence of DLAEYSGTYSSPADSPAGYDY (SEQ ID NO: 223);
v) the CDR1 sequence of TTYRMG (SEQ ID NO: 172), the CDR2 sequence of FVAAIRWSGGRTL (SEQ ID NO: 202), and the CDR3 sequence of AADLAEYSGTYSSPADSPAGYD (SEQ ID NO: 234); or
vi) the CDR1 sequence of GRTFTTYRMG (SEQ ID NO: 182), the CDR2 sequence of AIRWSGGRTL (SEQ ID NO: 212), and the CDR3 sequence of DLAEYSGTYSSPADSPAGYDY (SEQ ID NO: 245); and
k) VHH12:
i) the CDR1 sequence of FNTYAMG (SEQ ID NO: 9), the CDR2 sequence of SITWNGGSTSYADSVKG (SEQ ID NO: 39), and the CDR3 sequence of ARYYVSGTYFPANY (SEQ ID NO: 70);
ii) the CDR1 sequence of GRTLSFNTY (SEQ ID NO: 19), the CDR2 sequence of TWNGGS (SEQ ID NO: 49) or WNGG (SEQ ID NO: 269), and the CDR3 sequence of ARYYVSGTYFPANY (SEQ ID NO: 81) or RYYVSGTYFPAN (SEQ ID NO: 282);
iii) the CDR1 sequence of GRTLSFNTYA (SEQ ID NO: 29), the CDR2 sequence of ITWNGGST (SEQ ID NO: 59), and the CDR3 sequence of AAARYYVSGTYFPANY (SEQ ID NO: 92);
iv) the CDR1 sequence of GRTLSFNTYAMG (SEQ ID NO: 163), the CDR2 sequence of SITWNGGSTSYADSVKG (SEQ ID NO: 193), and the CDR3 sequence of ARYYVSGTYFPANY (SEQ ID NO: 224);
v) the CDR1 sequence of SFNTYAMG (SEQ ID NO: 173), the CDR2 sequence of FVASITWNGGSTS (SEQ ID NO: 203), and the CDR3 sequence of AAARYYVSGTYFPAN (SEQ ID NO: 235); or
vi) the CDR1 sequence of GRTLSFNTYAMG (SEQ ID NO: 183), the CDR2 sequence of SITWNGGSTS (SEQ ID NO: 213), and the CDR3 sequence of ARYYVSGTYFPANY (SEQ ID NO: 246).

123. The method of any one of claims 94 to 121, wherein the VHH domain comprises a
(SEQ ID NO: 93)
QVQLVESGGGLVQAGGSLKLACAAPGLTFSSYRMGWFRQAPGQEREFVA
AIDWNGRGTYYRYYADSVKGRSTISRDNAKNTMYLQMNSLKPEDTAVYY
CAAGSIDLNWYGGMDYWGQGTQVTVSS,
(SEQ ID NO: 94)
EVQVVESGGGLVQAGGSLKLACAAPGLTFSSYRMGWFRQAPGQEREFVA
AIDWNGRGTYYRYYADSVKGRSTISRDNAKNTVYLQMNSLKPEDTAVYY
CAATTVLTDPRVLNEYATWGQGTQVTVSS,
(SEQ ID NO: 95)
QLQLVESGGGLVQPGGSLRLSCAASGSIFSINVMGWYRQAPGKQRELVA
RINGGGITHYAESVKGRFTISRDNAKNTVYLQMNSLKPEDTAAYYCKAD
VFGSSGYVETYWGQGTQVTVSS,
(SEQ ID NO: 96)
EVQVVESGGGLVQAGGSLRLSCAVSGTSVSSNAMGWYRQAPGKQREWVG
FIDRIATTTIATSVKGRFAITRDNAKNTVYLQMSGLKPEDTAVYYCNHP
LTARWGQGTQVTVSS,
(SEQ ID NO: 97)
QVQLVESGGGLVQAGGSLRLSCAASGRTFSSYAMGWFRQAPGKEREFVA
AITWNGGTTYYADSVKGRFTISRDNAKNTVYLQMNSLKPEDTAVYYCAA
DPFNQGYWGQGTQVTVSS,
(SEQ ID NO: 98)
EVQLVESGGGLVQAGGSLRLSCAVSGSSVSSDAMGWYRQAPGNQRAWVA
FISGGGTTTYADSVKGRFTISRDNTKNTVYLHMNSLKPEDTAVYYCNHP
LTSRWGQGTQVTVSS,
(SEQ ID NO: 99)
EVQVVESGGGLVQAGGSLRLACVASRSIGSINVMGWYRQAPGKQRDLVA
RITGGGSTHYAESVKGRFTISRDNAKNTVYLQMNSLEPEDTAVYYCASM
VNPIITAWGTIGVREIPDYDYWGQGTQVTVSS,
(SEQ ID NO: 100)
QVQLVESGGGLVQAGGSLRLSCAVSGRTFSTYRMGWFRQAPGKERSFVA
AISWSGGSTTYADPVKGRFTISRDNAKNTVYLRMNSLKPEDTAVYYCND
QRGYWGQGTLVTVSS,
(SEQ ID NO: 101)
EVQVVESGGGLVQAGGSLRLSCAASGFTFTRYAMGWFRQAPGKERSFVA
AISWSGSSAGYGDSVKGRFTISRDNAKNTLYLQMNSLKPEDTAVYYCAA
DPFNQGYWGQGTQVTVSS,
(SEQ ID NO: 102)
EVQVVESGGGLVQAGGSLRLSCAASGRTFTTYRMGWFRQAPGKEREFVA
AIRWSGGRTLYADSVKGRFTISRDNAKNTAYLQMNNLRPEDTAVYYCAA
DLAEYSGTYSSPADSPAGYDYWGQGTQVTVSS,
or
(SEQ ID NO: 103)
QVQLVETGGGLVQAGDSLRLSCAASGRTLSFNTYAMGWFRQAPGKEREF
VASITWNGGSTSYADSVKGRFTITRDNAKNTATLRMNSLQPDDTAVYYC
AAARYYVSGTYFPANYWGQGTQVTVSS.

124. The method of any one of claims 94 to 121, wherein the VHH domain comprises a framework comprising sequence having at least 75%, 80%, 85%, 90%, 95%, 96%, 97%, 98%, 99%, or 100% sequence identity with the sequence of
(SEQ ID NO: 93)
QVQLVESGGGLVQAGGSLKLACAAPGLTFSSYRMGWFRQAPGQEREFVA
AIDWNGRGTYYRYYADSVKGRSTISRDNAKNTMYLQMNSLKPEDTAVYY
CAAGSIDLNWYGGMDYWGQGTQVTVSS,
(SEQ ID NO: 94)
EVQVVESGGGLVQAGGSLKLACAAPGLTFSSYRMGWFRQAPGQEREFVA
AIDWNGRGTYYRYYADSVKGRSTISRDNAKNTVYLQMNSLKPEDTAVYY
CAATTVLTDPRVLNEYATWGQGTQVTVSS,
(SEQ ID NO: 95)
QLQLVESGGGLVQPGGSLRLSCAASGSIFSINVMGWYRQAPGKQRELVA
RINGGGITHYAESVKGRFTISRDNAKNTVYLQMNSLKPEDTAAYYCKAD
VFGSSGYVETYWGQGTQVTVSS,
(SEQ ID NO: 96)
EVQVVESGGGLVQAGGSLRLSCAVSGTSVSSNAMGWYRQAPGKQREWVG
FIDRIATTTIATSVKGRFAITRDNAKNTVYLQMSGLKPEDTAVYYCNHP
LTARWGQGTQVTVSS,
(SEQ ID NO: 97)
QVQLVESGGGLVQAGGSLRLSCAASGRTFSSYAMGWFRQAPGKEREFVA
AITWNGGTTYYADSVKGRFTISRDNAKNTVYLQMNSLKPEDTAVYYCAA
DPFNQGYWGQGTQVTVSS,
(SEQ ID NO: 98)
EVQLVESGGGLVQAGGSLRLSCAVSGSSVSSDAMGWYRQAPGNQRAWVA
FISGGGTTTYADSVKGRFTISRDNTKNTVYLHMNSLKPEDTAVYYCNHP
LTSRWGQGTQVTVSS,
(SEQ ID NO: 99)
EVQVVESGGGLVQAGGSLRLACVASRSIGSINVMGWYRQAPGKQRDLVA
RITGGGSTHYAESVKGRFTISRDNAKNTVYLQMNSLEPEDTAVYYCASM
VNPIITAWGTIGVREIPDYDYWGQGTQVTVSS,
(SEQ ID NO: 100)
QVQLVESGGGLVQAGGSLRLSCAVSGRTFSTYRMGWFRQAPGKERSFVA
AISWSGGSTTYADPVKGRFTISRDNAKNTVYLRMNSLKPEDTAVYYCND
QRGYWGQGTLVTVSS,
(SEQ ID NO: 101)
EVQVVESGGGLVQAGGSLRLSCAASGFTFTRYAMGWFRQAPGKERSFVA
AISWSGSSAGYGDSVKGRFTISRDNAKNTLYLQMNSLKPEDTAVYYCAA
DPFNQGYWGQGTQVTVSS,
(SEQ ID NO: 102)
EVQVVESGGGLVQAGGSLRLSCAASGRTFTTYRMGWFRQAPGKEREFVA
AIRWSGGRTLYADSVKGRFTISRDNAKNTAYLQMNNLRPEDTAVYYCAA
DLAEYSGTYSSPADSPAGYDYWGQGTQVTVSS,
or
(SEQ ID NO: 103)
QVQLVETGGGLVQAGDSLRLSCAASGRTLSFNTYAMGWFRQAPGKEREF
VASITWNGGSTSYADSVKGRFTITRDNAKNTATLRMNSLQPDDTAVYYC
AAARYYVSGTYFPANYWGQGTQVTVSS.

125. The method of any one of claims 94 to 124, wherein the VHH domain is comprised of a sequence having at least 75%, 80%, 85%, 90%, 95%, 96%, 97%, 98%, 99%, or 100% sequence identity with the sequence of
(SEQ ID NO: 93)
QVQLVESGGGLVQAGGSLKLACAAPGLTFSSYRMGWFRQAPGQEREFVA
AIDWNGRGTYYRYYADSVKGRSTISRDNAKNTMYLQMNSLKPEDTAVYY
CAAGSIDLNWYGGMDYWGQGTQVTVSS,
(SEQ ID NO: 94)
EVQVVESGGGLVQAGGSLKLACAAPGLTFSSYRMGWFRQAPGQEREFVA
AIDWNGRGTYYRYYADSVKGRSTISRDNAKNTVYLQMNSLKPEDTAVYY
CAATTVLTDPRVLNEYATWGQGTQVTVSS,
(SEQ ID NO: 95)
QLQLVESGGGLVQPGGSLRLSCAASGSIFSINVMGWYRQAPGKQRELVA
RINGGGITHYAESVKGRFTISRDNAKNTVYLQMNSLKPEDTAAYYCKAD
VFGSSGYVETYWGQGTQVTVSS,
(SEQ ID NO: 96)
EVQVVESGGGLVQAGGSLRLSCAVSGTSVSSNAMGWYRQAPGKQREWVG
FIDRIATTTIATSVKGRFAITRDNAKNTVYLQMSGLKPEDTAVYYCNHP
LTARWGQGTQVTVSS,
(SEQ ID NO: 97)
QVQLVESGGGLVQAGGSLRLSCAASGRTFSSYAMGWFRQAPGKEREFVA
AITWNGGTTYYADSVKGRFTISRDNAKNTVYLQMNSLKPEDTAVYYCAA
DPFNQGYWGQGTQVTVSS,
(SEQ ID NO: 98)
EVQLVESGGGLVQAGGSLRLSCAVSGSSVSSDAMGWYRQAPGNQRAWVA
FISGGGTTTYADSVKGRFTISRDNTKNTVYLHMNSLKPEDTAVYYCNHP
LTSRWGQGTQVTVSS,
(SEQ ID NO: 99)
EVQVVESGGGLVQAGGSLRLACVASRSIGSINVMGWYRQAPGKQRDLVA
RITGGGSTHYAESVKGRFTISRDNAKNTVYLQMNSLEPEDTAVYYCASM
VNPIITAWGTIGVREIPDYDYWGQGTQVTVSS,
(SEQ ID NO: 100)
QVQLVESGGGLVQAGGSLRLSCAVSGRTFSTYRMGWFRQAPGKERSFVA
AISWSGGSTTYADPVKGRFTISRDNAKNTVYLRMNSLKPEDTAVYYCND
QRGYWGQGTLVTVSS,
(SEQ ID NO: 101)
EVQVVESGGGLVQAGGSLRLSCAASGFTFTRYAMGWFRQAPGKERSFVA
AISWSGSSAGYGDSVKGRFTISRDNAKNTLYLQMNSLKPEDTAVYYCAA
DPFNQGYWGQGTQVTVSS,
(SEQ ID NO: 102)
EVQVVESGGGLVQAGGSLRLSCAASGRTFTTYRMGWFRQAPGKEREFVA
AIRWSGGRTLYADSVKGRFTISRDNAKNTAYLQMNNLRPEDTAVYYCAA
DLAEYSGTYSSPADSPAGYDYWGQGTQVTVSS,
or
(SEQ ID NO: 103)
QVQLVETGGGLVQAGDSLRLSCAASGRTLSFNTYAMGWFRQAPGKEREF
VASITWNGGSTSYADSVKGRFTITRDNAKNTATLRMNSLQPDDTAVYYC
AAARYYVSGTYFPANYWGQGTQVTVSS.

126. The method of any one of claims 94 to 125, wherein the agent is an antibody or fragment thereof, a peptide, a vaccine, a small molecule, a polynucleotide, a radioisotope, a toxin, an enzyme, an anticoagulant, a hormone, a cytokine, an anti-inflammatory molecule, an RNAi, an mRNA, a self-replicating RNA, an antibiotic, or an antibody-antibiotic conjugate.

127. The method of claim 126, wherein the agent is an antibiotic, an antibody or fragment thereof, a peptide or a vaccine.

128. The method of any one of claims 125 to 127, wherein the VHH domain is genetically fused or chemically conjugated to the agent.

129. The method of claim 128, further comprising a linker between the VHH domain and the agent.

130. The method of claim 129, wherein the linker is a polypeptide.

131. The method of claim 130, wherein the linker is a flexible linker comprising a sequence selected from the group consisting of EPKTPKPQPQPQLQPQPNPTTESKSPK (SEQ ID NO: 130), (EAAAK)n (SEQ ID NO: 147), (GGGGS)n (SEQ ID NO: 148) and (GGGS)n (SEQ ID NO: 149), wherein n is an integer from 1 to 20.

132. The method of any one of claims 125 to 131, wherein the VHH domain is chemically-conjugated to the agent.

133. The method of any one of claims 125 to 131, wherein the VHH domain is non-covalently bound to the agent.

134. The method of any one of claims 94 to 133, wherein the method does not inhibit pIgR-mediated transcytosis of IgA.

135. The method of claim 134, wherein the VHH domain comprises a CDR1 sequence of SNAMG (SEQ ID NO: 3), INVMG (SEQ ID NO: 6), TYRMG (SEQ ID NO: 7), RYAMG (SEQ ID NO: 8), FTTYRMG (SEQ ID NO: 258), TYRMG (SEQ ID NO: 259), FNTYAMG (SEQ ID NO: 9), GTSVSSN (SEQ ID NO: 12), GRTFSSY (SEQ ID NO: 13), RSIGSIN (SEQ ID NO: 15), GRTFSTY (SEQ ID NO: 16), GFTFTRY (SEQ ID NO: 17), GRTFTTY (SEQ ID NO: 18), GRTLSFNTY (SEQ ID NO: 19), GTSVSSNA (SEQ ID NO: 22), RSIGSINV (SEQ ID NO: 25), GRTFSTYR (SEQ ID NO: 26), GFTFTRYA (SEQ ID NO: 27), GRTFTTYR (SEQ ID NO: 28), GRTLSFNTYA (SEQ ID NO: 29), GTSVSSNAMG (SEQ ID NO: 156), RSIGSINVMG (SEQ ID NO: 159), GRTFSTYRMG (SEQ ID NO: 160), GFTFTRYAMG (SEQ ID NO: 161), GRTFTTYRMG (SEQ ID NO: 162), GRTLSFNTYAMG (SEQ ID NO: 163), SSNAMG (SEQ ID NO: 166), SINVMG (SEQ ID NO: 169), STYRMG (SEQ ID NO: 170), TRYAMG (SEQ ID NO: 171), TTYRMG (SEQ ID NO: 172), SFNTYAMG (SEQ ID NO: 173), GTSVSSNAMG (SEQ ID NO: 176), RSIGSINVMG (SEQ ID NO: 179), GRTFSTYRMG (SEQ ID NO: 180), GFTFTRYAMG (SEQ ID NO: 181), GRTFTTYRMG (SEQ ID NO: 182), or GRTLSFNTYAMG (SEQ ID NO: 183).

136. The method of claim 134 or claim 135, wherein the VHH domain comprises a CDR2 sequence of FIDRIATTTIATSVKG (SEQ ID NO: 32), RITGGGSTHYAESVKG (SEQ ID NO: 35), AISWSGGSTTYADPVKG (SEQ ID NO: 36), AISWSGSSAGYGDSVKG (SEQ ID NO: 37), AIRWSGGRTLYADSVKG (SEQ ID NO: 38), SITWNGGSTSYADSVKG (SEQ ID NO: 39), DRIAT (SEQ ID NO: 42), RIA (SEQ ID NO: 262), TGGGS (SEQ ID NO: 45), GGG (SEQ ID NO: 265), SWSGGS (SEQ ID NO: 46), WSGG (SEQ ID NO: 266), SWSGSS (SEQ ID NO: 47), WSGS (SEQ ID NO: 267), RWSGGR (SEQ ID NO: 48), WSGG (SEQ ID NO: 268), TWNGGS (SEQ ID NO: 49), WNGG (SEQ ID NO: 269), IDRIATT (SEQ ID NO: 52), ITGGGST (SEQ ID NO: 55), ISWSGGST (SEQ ID NO: 56), ISWSGSSA (SEQ ID NO: 57), IRWSGGRT (SEQ ID NO: 58), ITWNGGST (SEQ ID NO: 59), FIDRIATTTIATSVKG (SEQ ID NO: 186), RITGGGSTHYAESVKG (SEQ ID NO: 189), AISWSGGSTTYADPVKG (SEQ ID NO: 190), AISWSGSSAGYGDSVKG (SEQ ID NO: 191), AIRWSGGRTLYADSVKG (SEQ ID NO: 192), SITWNGGSTSYADSVKG (SEQ ID NO: 193), WVGFIDRIATTT (SEQ ID NO: 196), LVARITGGGSTH (SEQ ID NO: 199), FVAAISWSGGSTT (SEQ ID NO: 200), FVAAISWSGSSAG (SEQ ID NO: 201), FVAAIRWSGGRTL (SEQ ID NO: 202), FVASITWNGGSTS (SEQ ID NO: 203), FIDRIATTT (SEQ ID NO: 206), RITGGGSTH (SEQ ID NO: 209), AISWSGGSTT (SEQ ID NO: 210), AISWSGSSAG (SEQ ID NO: 211), AIRWSGGRTL (SEQ ID NO: 212), or SITWNGGSTS (SEQ ID NO: 213).

137. The method of any one of claims 134 to 136, wherein the VHH domain comprises a CDR3 sequence of PLTAR (SEQ ID NO: 63), MVNPIITAWGTIGVREIPDYDY (SEQ ID NO: 66), DQRGY (SEQ ID NO: 67), QRGY (SEQ ID NO: 271), DPFNQGY (SEQ ID NO: 68), DLAEYSGTYSSPADSPAGYDY (SEQ ID NO: 69), ARYYVSGTYFPANY (SEQ ID NO: 70), PLTAR (SEQ ID NO: 74), LTA (SEQ ID NO: 275), MVNPIITAWGTIGVREIPDYDY (SEQ ID NO: 77), VNPIITAWGTIGVREIPDYD (SEQ ID NO: 278), DQRGY (SEQ ID NO: 78), RG (SEQ ID NO: 279), DPFNQGY (SEQ ID NO: 79), PFNQG (SEQ ID NO: 280), DLAEYSGTYSSPADSPAGYDY (SEQ ID NO: 80), LAEYSGTYSSPADSPAGYD (SEQ ID NO: 281), ARYYVSGTYFPANY (SEQ ID NO: 81), RYYVSGTYFPAN (SEQ ID NO: 282), NHPLTAR (SEQ ID NO: 85), ASMVNPIITAWGTIGVREIPDYDY (SEQ ID NO: 88), NDQRGY (SEQ ID NO: 89), AADPFNQGY (SEQ ID NO: 90), AADLAEYSGTYSSPADSPAGYDY (SEQ ID NO: 91), AAARYYVSGTYFPANY (SEQ ID NO: 92), PLTAR (SEQ ID NO: 217), MVNPIITAWGTIGVREIPDYDY (SEQ ID NO: 220), QRGY (SEQ ID NO: 221), DPFNQGY (SEQ ID NO: 222), DLAEYSGTYSSPADSPAGYDY (SEQ ID NO: 223), ARYYVSGTYFPANY (SEQ ID NO: 224), NHPLTA (SEQ ID NO: 228), ASMVNPIITAWGTIGVREIPDYD (SEQ ID NO: 231), NDQRG (SEQ ID NO: 232), AADPFNQG (SEQ ID NO: 233), AADLAEYSGTYSSPADSPAGYD (SEQ ID NO: 234), AAARYYVSGTYFPAN (SEQ ID NO: 235), PLTAR (SEQ ID NO: 239), MVNPIITAWGTIGVREIPDYDY (SEQ ID NO: 242), QRGY (SEQ ID NO: 243), DPFNQGY (SEQ ID NO: 244), DLAEYSGTYSSPADSPAGYDY (SEQ ID NO: 245), or ARYYVSGTYFPANY (SEQ ID NO: 246).

138. A method to diagnose a disease or condition comprising administering to the subject (i) a VHH domain that binds to an extracellular domain 1, an extracellular domain 2, an extracellular domain 1-2, an extracellular domain 3, an extracellular domain 2-3, an extracellular domain 4-5, or an extracellular domain 5 of polymeric immunoglobulin receptor (pIgR), or (ii) a therapeutic molecule comprising an agent and the VHH domain, to the subject, the method comprising detecting the amount of VHH domain in a tissue of the subject, wherein the tissue comprises a diseased cell, and comparing the amount of VHH domain in the tissue of the subject with a reference amount of VHH domain in the tissue of a comparable healthy subject.

139. The method of claim 138, wherein the tissue comprises a mucosal cell.

140. The method of claim 138, wherein the tissue comprises a mucosal lumen.

141. The method of claim 138, wherein the VHH domain competes with IgA binding to the pIgR.

142. The method of claim 138, wherein the VHH domain promotes IgA binding to the pIgR.

143. The method of any one of claims 138 to 142, wherein the K_{D} of the binding of the VHH domain to pIgR is from about 4 to about 525 nM.

144. The method of any one of claims 138 to 143, wherein the K_{D} of the binding of the VHH domain to pIgR is less than about 50 nM.

145. The method of any one of claims 138 to 144, wherein the K_{D} of the binding of the VHH domain to pIgR is from about 4 to about 34 nM.

146. The method of any one of claims 138 to 145, wherein the Tₘ of the VHH domain is from about 53 to about 77° C.

147. The method of any one of claims 138 to 146, wherein the Tₘ of the VHH domain is from 53.9 to 76.4° C.

148. The method of any one of claims 138 to 147, wherein the VHH domain binds to the extracellular domain 1 of pIgR.

149. The method of any one of claims 138 to 147, wherein the VHH domain binds to the extracellular domain 2 of pIgR.

150. The method of any one of claims 138 to 147, wherein the VHH domain binds to the extracellular domain 1-2 of pIgR.

151. The method of any one of claims 138 to 147, wherein the VHH domain binds to the extracellular domain 3 of pIgR.

152. The method of any one of claims 138 to 147, wherein the VHH domain binds to the extracellular domain 2-3 of pIgR.

153. The method of any one of claims 138 to 147, wherein the VHH domain binds to the extracellular domain 4-5 of pIgR.

154. The method of any one of claims 138 to 147, wherein the VHH domain binds to the extracellular domain 5 of pIgR.

155. The method of any one of claims 138 to 154, wherein pIgR is human pIgR.

156. The method of any one of claims 138 to 154, wherein pIgR is mouse pIgR.

157. The method of any one of claims 138 to 154, wherein the VHH domain does not detectably bind to the amino acid sequence of EKAVADTRDQADGSRASVDSGSSEEQGGSSR (SEQ ID NO: 143), EREIQNVGDQAQENRASGDAGSADGQSRSSSSK (SEQ ID NO: 144), or EREIQNVRDQAQENRASGDAGSADGQSRSSSSK (SEQ ID NO: 145).

158. The method of any one of claims 138 to 157, wherein the VHH domain comprises a CDR3 sequence of GSIDLNWYGGMDY (SEQ ID NO: 60), TTVLTDPRVLNEYAT (SEQ ID NO: 61), DVFGSSGYVETY (SEQ ID NO: 62), PLTAR (SEQ ID NO: 63), DPFNQGY (SEQ ID NO: 64), PLTSR (SEQ ID NO: 65), MVNPIITAWGTIGVREIPDYDY (SEQ ID NO: 66), DQRGY (SEQ ID NO: 67), QRGY (SEQ ID NO: 271), DPFNQGY (SEQ ID NO: 68), DLAEYSGTYSSPADSPAGYDY (SEQ ID NO: 69), ARYYVSGTYFPANY (SEQ ID NO: 70), GSIDLNWYGGMDY (SEQ ID NO: 71), SIDLNWYGGMD (SEQ ID NO: 272), TTVLTDPRVLNEYAT (SEQ ID NO: 72), TVLTDPRVLNEYA (SEQ ID NO: 273), DVFGSSGYVETY (SEQ ID NO: 73), VFGSSGYVET (SEQ ID NO: 274), PLTAR (SEQ ID NO: 74), LTA (SEQ ID NO: 275), DPFNQGY (SEQ ID NO: 75), PFNQG (SEQ ID NO: 276), PLTSR (SEQ ID NO: 76), LTS (SEQ ID NO: 277), MVNPIITAWGTIGVREIPDYDY (SEQ ID NO: 77), VNPIITAWGTIGVREIPDYD (SEQ ID NO: 278), DQRGY (SEQ ID NO: 78), RG (SEQ ID NO: 279), DPFNQGY (SEQ ID NO: 79), PFNQG (SEQ ID NO: 280), DLAEYSGTYSSPADSPAGYDY (SEQ ID NO: 80), LAEYSGTYSSPADSPAGYD (SEQ ID NO: 281), ARYYVSGTYFPANY (SEQ ID NO: 81), RYYVSGTYFPAN (SEQ ID NO: 282), CAAGSIDLNWYGGMDY (SEQ ID NO: 82), AAGSIDLNWYGGMDY (SEQ ID NO: 283), CAATTVLTDPRVLNEYAT (SEQ ID NO: 83), AATTVLTDPRVLNEYAT (SEQ ID NO: 284), KADVFGSSGYVETY (SEQ ID NO: 84), NHPLTAR (SEQ ID NO: 85), AADPFNQGY (SEQ ID NO: 86), NHPLTSR (SEQ ID NO: 87), ASMVNPIITAWGTIGVREIPDYDY (SEQ ID NO: 88), NDQRGY (SEQ ID NO: 89), AADPFNQGY (SEQ ID NO: 90), AADLAEYSGTYSSPADSPAGYDY (SEQ ID NO: 91), AAARYYVSGTYFPANY (SEQ ID NO: 92), GSIDLNWYGGMDY (SEQ ID NO: 214), TTVLTDPRVLNEYAT (SEQ ID NO: 215), DVFGSSGYVETY (SEQ ID NO: 216), PLTAR (SEQ ID NO: 217), DPFNQGY (SEQ ID NO: 218), PLTSR (SEQ ID NO: 219), MVNPIITAWGTIGVREIPDYDY (SEQ ID NO: 220), QRGY (SEQ ID NO: 221), DPFNQGY (SEQ ID NO: 222), DLAEYSGTYSSPADSPAGYDY (SEQ ID NO: 223), ARYYVSGTYFPANY (SEQ ID NO: 224), AAGSIDLNWYGGMD (SEQ ID NO: 225), AATTVLTDPRVLNEYA (SEQ ID NO: 226), KADVFGSSGYVET (SEQ ID NO: 227), NHPLTA (SEQ ID NO: 228), AADPFNQG (SEQ ID NO: 229), NHPLTS (SEQ ID NO: 230), ASMVNPIITAWGTIGVREIPDYD (SEQ ID NO: 231), NDQRG (SEQ ID NO: 232), AADPFNQG (SEQ ID NO: 233), AADLAEYSGTYSSPADSPAGYD (SEQ ID NO: 234), AAARYYVSGTYFPAN (SEQ ID NO: 235), GSIDLNWYGGMDY (SEQ ID NO: 236), TTVLTDPRVLNEYAT (SEQ ID NO: 237), DVFGSSGYVETY (SEQ ID NO: 238), PLTAR (SEQ ID NO: 239), DPFNQGY (SEQ ID NO: 240), PLTSR (SEQ ID NO: 241), MVNPIITAWGTIGVREIPDYDY (SEQ ID NO: 242), QRGY (SEQ ID NO: 243), DPFNQGY (SEQ ID NO: 244), DLAEYSGTYSSPADSPAGYDY (SEQ ID NO: 245), or ARYYVSGTYFPANY (SEQ ID NO: 246).

159. The method of any one of claims 138 to 158, wherein the VHH domain comprises a CDR2 sequence of AIDWNGRGTYYRYYADSVKG (SEQ ID NO: 30), RINGGGITHYAESVKG (SEQ ID NO: 31), FIDRIATTTIATSVKG (SEQ ID NO: 32), AITWNGGTTYYADSVKG (SEQ ID NO: 33), FISGGGTTTYADSVKG (SEQ ID NO: 34), RITGGGSTHYAESVKG (SEQ ID NO: 35), AISWSGGSTTYADPVKG (SEQ ID NO: 36), AISWSGSSAGYGDSVKG (SEQ ID NO: 37), AIRWSGGRTLYADSVKG (SEQ ID NO: 38), SITWNGGSTSYADSVKG (SEQ ID NO: 39), DWNGRGTYY (SEQ ID NO: 40), WNGRGTY (SEQ ID NO: 260), NGGGI (SEQ ID NO: 41), GGG (SEQ ID NO: 261), DRIAT (SEQ ID NO: 42), RIA (SEQ ID NO: 262), TWNGGT (SEQ ID NO: 43), WNGG (SEQ ID NO: 263), SGGGT (SEQ ID NO: 44), GGG (SEQ ID NO: 264), TGGGS (SEQ ID NO: 45), GGG (SEQ ID NO: 265), SWSGGS (SEQ ID NO: 46), WSGG (SEQ ID NO: 266), SWSGSS (SEQ ID NO: 47), WSGS (SEQ ID NO: 267), RWSGGR (SEQ ID NO: 48), WSGG (SEQ ID NO: 268), TWNGGS (SEQ ID NO: 49), WNGG (SEQ ID NO: 269), IDWNGRGTYY (SEQ ID NO: 50), IDWNGRGTYYR (SEQ ID NO: 270), INGGGIT (SEQ ID NO: 51), IDRIATT (SEQ ID NO: 52), ITWNGGTT (SEQ ID NO: 53), ISGGGTT (SEQ ID NO: 54), ITGGGST (SEQ ID NO: 55), ISWSGGST (SEQ ID NO: 56), ISWSGSSA (SEQ ID NO: 57), IRWSGGRT (SEQ ID NO: 58), ITWNGGST (SEQ ID NO: 59), AIDWNGRGTYYRYYADSVKG (SEQ ID NO: 184), RINGGGITHYAESVKG (SEQ ID NO: 185), FIDRIATTTIATSVKG (SEQ ID NO: 186), AITWNGGTTYYADSVKG (SEQ ID NO: 187), FISGGGTTTYADSVKG (SEQ ID NO: 188), RITGGGSTHYAESVKG (SEQ ID NO: 189), AISWSGGSTTYADPVKG (SEQ ID NO: 190), AISWSGSSAGYGDSVKG (SEQ ID NO: 191), AIRWSGGRTLYADSVKG (SEQ ID NO: 192), SITWNGGSTSYADSVKG (SEQ ID NO: 193), FVAAIDWNGRGTYYRY (SEQ ID NO: 194), LVARINGGGITH (SEQ ID NO: 195), WVGFIDRIATTT (SEQ ID NO: 196), FVAAITWNGGTTY (SEQ ID NO: 197), WVAFISGGGTTT (SEQ ID NO: 198), LVARITGGGSTH (SEQ ID NO: 199), FVAAISWSGGSTT (SEQ ID NO: 200), FVAAISWSGSSAG (SEQ ID NO: 201), FVAAIRWSGGRTL (SEQ ID NO: 202), FVASITWNGGSTS (SEQ ID NO: 203), AIDWNGRGTYYRY (SEQ ID NO: 204), RINGGGITH (SEQ ID NO: 205), FIDRIATTT (SEQ ID NO: 206), AITWNGGTTY (SEQ ID NO: 207), FISGGGTTT (SEQ ID NO: 208), RITGGGSTH (SEQ ID NO: 209), AISWSGGSTT (SEQ ID NO: 210), AISWSGSSAG (SEQ ID NO: 211), AIRWSGGRTL (SEQ ID NO: 212), or SITWNGGSTS (SEQ ID NO: 213).

160. The method of any one of claims 138 to 159, wherein the VHH domain comprises a CDR1 sequence of SYRMG (SEQ ID NO: 1), INVMG (SEQ ID NO: 2), SNAMG (SEQ ID NO: 3), SYAMG (SEQ ID NO: 4), SDAMG (SEQ ID NO: 5), INVMG (SEQ ID NO: 6), TYRMG (SEQ ID NO: 7), RYAMG (SEQ ID NO: 8), FTTYRMG (SEQ ID NO: 258), TYRMG (SEQ ID NO: 259), FNTYAMG (SEQ ID NO: 9), GLTFSSY (SEQ ID NO: 10), GSIFSIN (SEQ ID NO: 11), GTSVSSN (SEQ ID NO: 12), GRTFSSY (SEQ ID NO: 13), GSSVSSD (SEQ ID NO: 14), RSIGSIN (SEQ ID NO: 15), GRTFSTY (SEQ ID NO: 16), GFTFTRY (SEQ ID NO: 17), GRTFTTY (SEQ ID NO: 18), GRTLSFNTY (SEQ ID NO: 19), GLTFSSYR (SEQ ID NO: 20), GSIFSINV (SEQ ID NO: 21), GTSVSSNA (SEQ ID NO: 22), GRTFSSYA (SEQ ID NO: 23), GSSVSSDA (SEQ ID NO: 24), RSIGSINV (SEQ ID NO: 25), GRTFSTYR (SEQ ID NO: 26), GFTFTRYA (SEQ ID NO: 27), GRTFTTYR (SEQ ID NO: 28), GRTLSFNTYA (SEQ ID NO: 29), GLTFSSYRMG (SEQ ID NO: 154), GSIFSINVMG (SEQ ID NO: 155), GTSVSSNAMG (SEQ ID NO: 156), GRTFSSYAMG (SEQ ID NO: 157), GSSVSSDAMG (SEQ ID NO: 158), RSIGSINVMG (SEQ ID NO: 159), GRTFSTYRMG (SEQ ID NO: 160), GFTFTRYAMG (SEQ ID NO: 161), GRTFTTYRMG (SEQ ID NO: 162), GRTLSFNTYAMG (SEQ ID NO: 163), SSYRMG (SEQ ID NO: 164), SINVMG (SEQ ID NO: 165), SSNAMG (SEQ ID NO: 166), SSYAMG (SEQ ID NO: 167), SSDAMG (SEQ ID NO: 168), SINVMG (SEQ ID NO: 169), STYRMG (SEQ ID NO: 170), TRYAMG (SEQ ID NO: 171), TTYRMG (SEQ ID NO: 172), SFNTYAMG (SEQ ID NO: 173), GLTFSSYRMG (SEQ ID NO: 174), GSIFSINVMG (SEQ ID NO: 175), GTSVSSNAMG (SEQ ID NO: 176), GRTFSSYAMG (SEQ ID NO: 177), GSSVSSDAMG (SEQ ID NO: 178), RSIGSINVMG (SEQ ID NO: 179), GRTFSTYRMG (SEQ ID NO: 180), GFTFTRYAMG (SEQ ID NO: 181), GRTFTTYRMG (SEQ ID NO: 182), or GRTLSFNTYAMG (SEQ ID NO: 183).

161. The method of any one of claims 138 to 160, wherein the VHH domain comprises a CDR1 sequence, a CDR2 sequence, and a CDR3 sequence of the VHH domain selected from the group consisting of:
a) VHH1:
i) the CDR1 sequence of SYRMG (SEQ ID NO: 1), the CDR2 sequence of AIDWNGRGTYYRYYADSVKG (SEQ ID NO: 30), and the CDR3 sequence of GSIDLNWYGGMDY (SEQ ID NO: 60);
ii) the CDR1 sequence of GLTFSSY (SEQ ID NO: 10), the CDR2 sequence of DWNGRGTYY (SEQ ID NO: 40) or WNGRGTY (SEQ ID NO: 260), and the CDR3 sequence of GSIDLNWYGGMDY (SEQ ID NO: 71) or SIDLNWYGGMD (SEQ ID NO: 272);
iii) the CDR1 sequence of GLTFSSYR (SEQ ID NO: 20), the CDR2 sequence of IDWNGRGTYY (SEQ ID NO: 50) or IDWNGRGTYYR (SEQ ID NO: 270), and the CDR3 sequence of CAAGSIDLNWYGGMDY (SEQ ID NO: 82) or AAGSIDLNWYGGMDY (SEQ ID NO: 283);
iv) the CDR1 sequence of GLTFSSYRMG (SEQ ID NO: 154), the CDR 2 sequence of AIDWNGRGTYYRYYADSVKG (SEQ ID NO: 184), and the CDR3 sequence of GSIDLNWYGGMDY (SEQ ID NO: 214);
v) the CDR1 sequence of SSYRMG (SEQ ID NO: 164), the CDR2 sequence of FVAAIDWNGRGTYYRY (SEQ ID NO: 194), and the CDR3 sequence of AAGSIDLNWYGGMD (SEQ ID NO: 225); or
vi) the CDR1 sequence of GLTFSSYRMG (SEQ ID NO: 174), the CDR2 sequence of AIDWNGRGTYYRY (SEQ ID NO: 204), and the CDR3 sequence of GSIDLNWYGGMDY (SEQ ID NO: 236);
b) VHH2:
i) the CDR1 sequence of SYRMG (SEQ ID NO: 1), the CDR2 sequence of AIDWNGRGTYYRYYADSVKG (SEQ ID NO: 30), and the CDR3 sequence of TTVLTDPRVLNEYAT (SEQ ID NO: 61);
ii) the CDR1 sequence of GLTFSSY (SEQ ID NO: 10), the CDR2 sequence of DWNGRGTYY (SEQ ID NO: 40) or WNGRGTY (SEQ ID NO: 260), and the CDR3 sequence of TTVLTDPRVLNEYAT (SEQ ID NO: 72) or TVLTDPRVLNEYA (SEQ ID NO: 273);
iii) the CDR1 sequence of GLTFSSYR (SEQ ID NO: 20), the CDR2 sequence of IDWNGRGTYY (SEQ ID NO: 50) or IDWNGRGTYYR (SEQ ID NO: 270), and the CDR3 sequence of CAATTVLTDPRVLNEYAT (SEQ ID NO: 83) or AATTVLTDPRVLNEYAT (SEQ ID NO: 284);
iv) the CDR1 sequence of GLTFSSYRMG (SEQ ID NO: 154), the CDR2 sequence of AIDWNGRGTYYRYYADSVKG (SEQ ID NO: 184), and the CDR3 sequence of TTVLTDPRVLNEYAT (SEQ ID NO: 215);
v) the CDR1 sequence of SSYRMG (SEQ ID NO: 164), the CDR2 sequence of FVAAIDWNGRGTYYRY (SEQ ID NO: 194), and the CDR3 sequence of AATTVLTDPRVLNEYA (SEQ ID NO: 226); or
vi) the CDR1 sequence of GLTFSSYRMG (SEQ ID NO: 174), the CDR2 sequence of AIDWNGRGTYYRY (SEQ ID NO: 204), and the CDR3 sequence of TTVLTDPRVLNEYAT (SEQ ID NO: 237);
c) VHH3:
i) the CDR1 sequence of INVMG (SEQ ID NO: 2), the CDR2 sequence of RINGGGITHYAESVKG (SEQ ID NO: 31), and the CDR3 sequence of DVFGSSGYVETY (SEQ ID NO: 62);
ii) the CDR1 sequence of GSIFSIN (SEQ ID NO: 11), the CDR2 sequence of NGGGI (SEQ ID NO: 41) or GGG (SEQ ID NO: 261), and the CDR3 sequence of DVFGSSGYVETY (SEQ ID NO: 73) or VFGSSGYVET (SEQ ID NO: 274);
iii) the CDR1 sequence of GSIFSINV (SEQ ID NO: 21), the CDR2 sequence of INGGGIT (SEQ ID NO: 51), and the CDR3 sequence of KADVFGSSGYVETY (SEQ ID NO: 84);
iv) the CDR1 sequence of GSIFSINVMG (SEQ ID NO: 155), the CDR2 sequence of RINGGGITHYAESVKG (SEQ ID NO: 185), and the CDR3 sequence of DVFGSSGYVETY (SEQ ID NO: 216);
v) the CDR1 sequence of SINVMG (SEQ ID NO: 165), the CDR2 sequence of LVARINGGGITH (SEQ ID NO: 195), and the CDR3 sequence of KADVFGSSGYVET (SEQ ID NO: 227); or
vi) the CDR1 sequence of GSIFSINVMG (SEQ ID NO: 175), the CDR2 sequence of RINGGGITH (SEQ ID NO: 205), and the CDR3 sequence of DVFGSSGYVETY (SEQ ID NO: 238);
d) VHH4:
i) the CDR1 sequence of SNAMG (SEQ ID NO: 3), the CDR2 sequence of FIDRIATTTIATSVKG (SEQ ID NO: 32), and the CDR3 sequence of PLTAR (SEQ ID NO: 63);
ii) the CDR1 sequence of GTSVSSN (SEQ ID NO: 12), the CDR2 sequence of DRIAT (SEQ ID NO: 42) or RIA (SEQ ID NO: 262), and the CDR3 sequence of PLTAR (SEQ ID NO: 74) or LTA (SEQ ID NO: 275);
iii) the CDR1 sequence of GTSVSSNA (SEQ ID NO: 22), the CDR2 sequence of IDRIATT (SEQ ID NO: 52), and the CDR3 sequence of NHPLTAR (SEQ ID NO: 85);
iv) the CDR1 sequence of GTSVSSNAMG (SEQ ID NO: 156), the CDR2 sequence of FIDRIATTTIATSVKG (SEQ ID NO: 186), and the CDR3 sequence of PLTAR (SEQ ID NO: 217);
v) the CDR1 sequence of SSNAMG (SEQ ID NO: 166), the CDR2 sequence of WVGFIDRIATTT (SEQ ID NO: 196), and the CDR3 sequence of NHPLTA (SEQ ID NO: 228); or
vi) the CDR1 sequence of GTSVSSNAMG (SEQ ID NO: 176), the CDR2 sequence of FIDRIATTT (SEQ ID NO: 206), and the CDR3 sequence of PLTAR (SEQ ID NO: 239);
e) VHH5:
i) the CDR1 sequence of SYAMG (SEQ ID NO: 4), the CDR2 sequence of AITWNGGTTYYADSVKG (SEQ ID NO: 33), and the CDR3 sequence of DPFNQGY (SEQ ID NO: 64);
ii) the CDR1 sequence of GRTFSSY (SEQ ID NO: 13), the CDR2 sequence of TWNGGT (SEQ ID NO: 43) or WNGG (SEQ ID NO: 263), and the CDR3 sequence of DPFNQGY (SEQ ID NO: 75) or PFNQG (SEQ ID NO: 276);
iii) the CDR1 sequence of GRTFSSYA (SEQ ID NO: 23), the CDR2 sequence of ITWNGGTT (SEQ ID NO: 53), and the CDR3 sequence of AADPFNQGY (SEQ ID NO: 86);
iv) the CDR1 sequence of GRTFSSYAMG (SEQ ID NO: 157), the CDR2 sequence of AITWNGGTTYYADSVKG (SEQ ID NO: 187), and the CDR3 sequence of DPFNQGY (SEQ ID NO: 218);
v) the CDR1 sequence of SSYAMG (SEQ ID NO: 167), the CDR2 sequence of FVAAITWNGGTTY (SEQ ID NO: 197), and the CDR3 sequence of AADPFNQG (SEQ ID NO: 229); or
vi) the CDR1 sequence of GRTFSSYAMG (SEQ ID NO: 177), the CDR2 sequence of AITWNGGTTY (SEQ ID NO: 207), and the CDR3 sequence of DPFNQGY (SEQ ID NO: 240);
f) VHH6:
i) the CDR1 sequence of SDAMG (SEQ ID NO: 5), the CDR2 sequence of FISGGGTTTYADSVKG (SEQ ID NO: 34), and the CDR3 sequence of PLTSR (SEQ ID NO: 65);
ii) the CDR1 sequence of GSSVSSD (SEQ ID NO: 14), the CDR2 sequence of SGGGT (SEQ ID NO: 44) or GGG (SEQ ID NO: 264), and the CDR3 sequence of PLTSR (SEQ ID NO: 76) or LTS (SEQ ID NO: 277);
iii) the CDR1 sequence of GSSVSSDA (SEQ ID NO: 24), the CDR2 sequence of ISGGGTT (SEQ ID NO: 54), and the CDR3 sequence of NHPLTSR (SEQ ID NO: 87);
iv) the CDR1 sequence of GSSVSSDAMG (SEQ ID NO: 158), the CDR2 sequence of FISGGGTTTYADSVKG (SEQ ID NO: 188), and the CDR3 sequence of PLTSR (SEQ ID NO: 219);
v) the CDR1 sequence of SSDAMG (SEQ ID NO: 168), the CDR2 sequence of WVAFISGGGTTT (SEQ ID NO: 198), and the CDR3 sequence of NHPLTS (SEQ ID NO: 230); or
vi) the CDR1 sequence of GSSVSSDAMG (SEQ ID NO: 178), the CDR2 sequence of FISGGGTTT (SEQ ID NO: 208), and the CDR3 sequence of PLTSR (SEQ ID NO: 241);
g) VHH7:
i) the CDR1 sequence of INVMG (SEQ ID NO: 6), the CDR2 sequence of RITGGGSTHYAESVKG (SEQ ID NO: 35), and the CDR3 sequence of MVNPIITAWGTIGVREIPDYDY (SEQ ID NO: 66);
ii) the CDR1 sequence of RSIGSIN (SEQ ID NO: 15), the CDR2 sequence of TGGGS (SEQ ID NO: 45) or GGG (SEQ ID NO: 265), and the CDR3 sequence of MVNPIITAWGTIGVREIPDYDY (SEQ ID NO: 77) or VNPIITAWGTIGVREIPDYD (SEQ ID NO: 278);
iii) the CDR1 sequence of RSIGSINV (SEQ ID NO: 25), the CDR2 sequence of ITGGGST (SEQ ID NO: 55), and the CDR3 sequence of ASMVNPIITAWGTIGVREIPDYDY (SEQ ID NO: 88);
iv) the CDR1 sequence of RSIGSINVMG (SEQ ID NO: 159), the CDR2 sequence of RITGGGSTHYAESVKG (SEQ ID NO: 189), and the CDR3 sequence of MVNPIITAWGTIGVREIPDYDY (SEQ ID NO: 220);
v) the CDR1 sequence of SINVMG (SEQ ID NO: 169), the CDR2 sequence of LVARITGGGSTH (SEQ ID NO: 199), and the CDR3 sequence of ASMVNPIITAWGTIGVREIPDYD (SEQ ID NO: 231); or
vi) the CDR1 sequence of RSIGSINVMG (SEQ ID NO: 179), the CDR2 sequence of RITGGGSTH (SEQ ID NO: 209), and the CDR3 sequence of MVNPIITAWGTIGVREIPDYDY (SEQ ID NO: 242);
h) VHH9:
i) the CDR1 sequence of TYRMG (SEQ ID NO: 7), the CDR2 sequence of AISWSGGSTTYADPVKG (SEQ ID NO: 36), and the CDR3 sequence of DQRGY (SEQ ID NO: 67) or QRGY (SEQ ID NO: 271);
ii) the CDR1 sequence of GRTFSTY (SEQ ID NO: 16), the CDR2 sequence of SWSGGS (SEQ ID NO: 46) or WSGG (SEQ ID NO: 266), and the CDR3 sequence of DQRGY (SEQ ID NO: 78) or RG (SEQ ID NO: 279);
iii) the CDR1 sequence of GRTFSTYR (SEQ ID NO: 26), the CDR2 sequence of ISWSGGST (SEQ ID NO: 56), and the CDR3 sequence of NDQRGY (SEQ ID NO: 89);
iv) the CDR1 sequence of GRTFSTYRMG (SEQ ID NO: 160), the CDR2 sequence of AISWSGGSTTYADPVKG (SEQ ID NO: 190), and the CDR3 sequence of QRGY (SEQ ID NO: 221);
v) the CDR1 sequence of STYRMG (SEQ ID NO: 170), the CDR2 sequence of FVAAISWSGGSTT (SEQ ID NO: 200), and the CDR3 sequence of NDQRG (SEQ ID NO: 232); or
vi) the CDR1 sequence of GRTFSTYRMG (SEQ ID NO: 180), the CDR2 sequence of AISWSGGSTT (SEQ ID NO: 210), and the CDR3 sequence of QRGY (SEQ ID NO: 243);
i) VHH10:
i) the CDR1 sequence of RYAMG (SEQ ID NO: 8), the CDR2 sequence of AISWSGSSAGYGDSVKG (SEQ ID NO: 37), and the CDR3 sequence of DPFNQGY (SEQ ID NO: 68);
ii) the CDR1 sequence of GFTFTRY (SEQ ID NO: 17), the CDR2 sequence of SWSGSS (SEQ ID NO: 47) or WSGS (SEQ ID NO: 267), and the CDR3 sequence of DPFNQGY (SEQ ID NO: 79) or PFNQG (SEQ ID NO: 280);
iii) the CDR1 sequence of GFTFTRYA (SEQ ID NO: 27), the CDR2 sequence of ISWSGSSA (SEQ ID NO: 57), and the CDR3 sequence of AADPFNQGY (SEQ ID NO: 90);
iv) the CDR1 sequence of GFTFTRYAMG (SEQ ID NO: 161), the CDR2 sequence of AISWSGSSAGYGDSVKG (SEQ ID NO: 191), and the CDR3 sequence of DPFNQGY (SEQ ID NO: 222);
v) the CDR1 sequence of TRYAMG (SEQ ID NO: 171), the CDR2 sequence of FVAAISWSGSSAG (SEQ ID NO: 201), and the CDR3 sequence of AADPFNQG (SEQ ID NO: 233); or
vi) the CDR1 sequence of GFTFTRYAMG (SEQ ID NO: 181), the CDR2 sequence of AISWSGSSAG (SEQ ID NO: 211), and the CDR3 sequence of DPFNQGY (SEQ ID NO: 244);
j) VHH11:
i) the CDR1 sequence of FTTYRMG (SEQ ID NO: 258) or TYRMG (SEQ ID NO: 259), the CDR2 sequence of AIRWSGGRTLYADSVKG (SEQ ID NO: 38), and the CDR3 sequence of DLAEYSGTYSSPADSPAGYDY (SEQ ID NO: 69);
ii) the CDR1 sequence of GRTFTTY (SEQ ID NO: 18), the CDR2 sequence of RWSGGR (SEQ ID NO: 48) or WSGG (SEQ ID NO: 268), and the CDR3 sequence of DLAEYSGTYSSPADSPAGYDY (SEQ ID NO: 80) or LAEYSGTYSSPADSPAGYD (SEQ ID NO: 281);
iii) the CDR1 sequence of GRTFTTYR (SEQ ID NO: 28), the CDR2 sequence of IRWSGGRT (SEQ ID NO: 58), and the CDR3 sequence of AADLAEYSGTYSSPADSPAGYDY (SEQ ID NO: 91);
iv) the CDR1 sequence of GRTFTTYRMG (SEQ ID NO: 162), the CDR2 sequence of AIRWSGGRTLYADSVKG (SEQ ID NO: 192), and the CDR3 sequence of DLAEYSGTYSSPADSPAGYDY (SEQ ID NO: 223);
v) the CDR1 sequence of TTYRMG (SEQ ID NO: 172), the CDR2 sequence of FVAAIRWSGGRTL (SEQ ID NO: 202), and the CDR3 sequence of AADLAEYSGTYSSPADSPAGYD (SEQ ID NO: 234); or
vi) the CDR1 sequence of GRTFTTYRMG (SEQ ID NO: 182), the CDR2 sequence of AIRWSGGRTL (SEQ ID NO: 212), and the CDR3 sequence of DLAEYSGTYSSPADSPAGYDY (SEQ ID NO: 245); and
k) VHH12:
i) the CDR1 sequence of FNTYAMG (SEQ ID NO: 9), the CDR2 sequence of SITWNGGSTSYADSVKG (SEQ ID NO: 39), and the CDR3 sequence of ARYYVSGTYFPANY (SEQ ID NO: 70);
ii) the CDR1 sequence of GRTLSFNTY (SEQ ID NO: 19), the CDR2 sequence of TWNGGS (SEQ ID NO: 49) or WNGG (SEQ ID NO: 269), and the CDR3 sequence of ARYYVSGTYFPANY (SEQ ID NO: 81) or RYYVSGTYFPAN (SEQ ID NO: 282);
iii) the CDR1 sequence of GRTLSFNTYA (SEQ ID NO: 29), the CDR2 sequence of ITWNGGST (SEQ ID NO: 59), and the CDR3 sequence of AAARYYVSGTYFPANY (SEQ ID NO: 92);
iv) the CDR1 sequence of GRTLSFNTYAMG (SEQ ID NO: 163), the CDR2 sequence of SITWNGGSTSYADSVKG (SEQ ID NO: 193), and the CDR3 sequence of ARYYVSGTYFPANY (SEQ ID NO: 224);
v) the CDR1 sequence of SFNTYAMG (SEQ ID NO: 173), the CDR2 sequence of FVASITWNGGSTS (SEQ ID NO: 203), and the CDR3 sequence of AAARYYVSGTYFPAN (SEQ ID NO: 235); or
vi) the CDR1 sequence of GRTLSFNTYAMG (SEQ ID NO: 183), the CDR2 sequence of SITWNGGSTS (SEQ ID NO: 213), and the CDR3 sequence of ARYYVSGTYFPANY (SEQ ID NO: 246).

162. The method of any one of claims 138 to 161, wherein the VHH domain comprises a framework derived from the framework of any of the VHH domains comprising the sequences of
(SEQ ID NO: 93)
QVQLVESGGGLVQAGGSLKLACAAPGLTFSSYRMGWFRQAPGQEREFVA
AIDWNGRGTYYRYYADSVKGRSTISRDNAKNTMYLQMNSLKPEDTAVYY
CAAGSIDLNWYGGMDYWGQGTQVTVSS,
(SEQ ID NO: 94)
EVQVVESGGGLVQAGGSLKLACAAPGLTFSSYRMGWFRQAPGQEREFVA
AIDWNGRGTYYRYYADSVKGRSTISRDNAKNTVYLQMNSLKPEDTAVYY
CAATTVLTDPRVLNEYATWGQGTQVTVSS,
(SEQ ID NO: 95)
QLQLVESGGGLVQPGGSLRLSCAASGSIFSINVMGWYRQAPGKQRELVA
RINGGGITHYAESVKGRFTISRDNAKNTVYLQMNSLKPEDTAAYYCKAD
VFGSSGYVETYWGQGTQVTVSS,
(SEQ ID NO: 96)
EVQVVESGGGLVQAGGSLRLSCAVSGTSVSSNAMGWYRQAPGKQREWVG
FIDRIATTTIATSVKGRFAITRDNAKNTVYLQMSGLKPEDTAVYYCNHP
LTARWGQGTQVTVSS,
(SEQ ID NO: 97)
QVQLVESGGGLVQAGGSLRLSCAASGRTFSSYAMGWFRQAPGKEREFVA
AITWNGGTTYYADSVKGRFTISRDNAKNTVYLQMNSLKPEDTAVYYCAA
DPFNQGYWGQGTQVTVSS,
(SEQ ID NO: 98)
EVQLVESGGGLVQAGGSLRLSCAVSGSSVSSDAMGWYRQAPGNQRAWVA
FISGGGTTTYADSVKGRFTISRDNTKNTVYLHMNSLKPEDTAVYYCNHP
LTSRWGQGTQVTVSS,
(SEQ ID NO: 99)
EVQVVESGGGLVQAGGSLRLACVASRSIGSINVMGWYRQAPGKQRDLVA
RITGGGSTHYAESVKGRFTISRDNAKNTVYLQMNSLEPEDTAVYYCASM
VNPIITAWGTIGVREIPDYDYWGQGTQVTVSS,
(SEQ ID NO: 100)
QVQLVESGGGLVQAGGSLRLSCAVSGRTFSTYRMGWFRQAPGKERSFVA
AISWSGGSTTYADPVKGRFTISRDNAKNTVYLRMNSLKPEDTAVYYCND
QRGYWGQGTLVTVSS,
(SEQ ID NO: 101)
EVQVVESGGGLVQAGGSLRLSCAASGFTFTRYAMGWFRQAPGKERSFVA
AISWSGSSAGYGDSVKGRFTISRDNAKNTLYLQMNSLKPEDTAVYYCAA
DPFNQGYWGQGTQVTVSS,
(SEQ ID NO: 102)
EVQVVESGGGLVQAGGSLRLSCAASGRTFTTYRMGWFRQAPGKEREFVA
AIRWSGGRTLYADSVKGRFTISRDNAKNTAYLQMNNLRPEDTAVYYCAA
DLAEYSGTYSSPADSPAGYDYWGQGTQVTVSS,
or
(SEQ ID NO: 103)
QVQLVETGGGLVQAGDSLRLSCAASGRTLSFNTYAMGWFRQAPGKEREF
VASITWNGGSTSYADSVKGRFTITRDNAKNTATLRMNSLQPDDTAVYYC
AAARYYVSGTYFPANYWGQGTQVTVSS.

163. The method of any one of claims 138 to 162, wherein the VHH domain comprises a framework comprising sequence having at least 75%, 80%, 85%, 90%, 95%, 96%, 97%, 98%, 99%, or 100% sequence identity with the sequence of
(SEQ ID NO: 93)
QVQLVESGGGLVQAGGSLKLACAAPGLTFSSYRMGWFRQAPGQEREFVA
AIDWNGRGTYYRYYADSVKGRSTISRDNAKNTMYLQMNSLKPEDTAVYY
CAAGSIDLNWYGGMDYWGQGTQVTVSS,
(SEQ ID NO: 94)
EVQVVESGGGLVQAGGSLKLACAAPGLTFSSYRMGWFRQAPGQEREFVA
AIDWNGRGTYYRYYADSVKGRSTISRDNAKNTVYLQMNSLKPEDTAVYY
CAATTVLTDPRVLNEYATWGQGTQVTVSS,
(SEQ ID NO: 95)
QLQLVESGGGLVQPGGSLRLSCAASGSIFSINVMGWYRQAPGKQRELVA
RINGGGITHYAESVKGRFTISRDNAKNTVYLQMNSLKPEDTAAYYCKAD
VFGSSGYVETYWGQGTQVTVSS,
(SEQ ID NO: 96)
EVQVVESGGGLVQAGGSLRLSCAVSGTSVSSNAMGWYRQAPGKQREWVG
FIDRIATTTIATSVKGRFAITRDNAKNTVYLQMSGLKPEDTAVYYCNHP
LTARWGQGTQVTVSS,
(SEQ ID NO: 97)
QVQLVESGGGLVQAGGSLRLSCAASGRTFSSYAMGWFRQAPGKEREFVA
AITWNGGTTYYADSVKGRFTISRDNAKNTVYLQMNSLKPEDTAVYYCAA
DPFNQGYWGQGTQVTVSS,
(SEQ ID NO: 98)
EVQLVESGGGLVQAGGSLRLSCAVSGSSVSSDAMGWYRQAPGNQRAWVA
FISGGGTTTYADSVKGRFTISRDNTKNTVYLHMNSLKPEDTAVYYCNHP
LTSRWGQGTQVTVSS,
(SEQ ID NO: 99)
EVQVVESGGGLVQAGGSLRLACVASRSIGSINVMGWYRQAPGKQRDLVA
RITGGGSTHYAESVKGRFTISRDNAKNTVYLQMNSLEPEDTAVYYCASM
VNPIITAWGTIGVREIPDYDYWGQGTQVTVSS,
(SEQ ID NO: 100)
QVQLVESGGGLVQAGGSLRLSCAVSGRTFSTYRMGWFRQAPGKERSFVA
AISWSGGSTTYADPVKGRFTISRDNAKNTVYLRMNSLKPEDTAVYYCND
QRGYWGQGTLVTVSS,
(SEQ ID NO: 101)
EVQVVESGGGLVQAGGSLRLSCAASGFTFTRYAMGWFRQAPGKERSFVA
AISWSGSSAGYGDSVKGRFTISRDNAKNTLYLQMNSLKPEDTAVYYCAA
DPFNQGYWGQGTQVTVSS,
(SEQ ID NO: 102)
EVQVVESGGGLVQAGGSLRLSCAASGRTFTTYRMGWFRQAPGKEREFVA
AIRWSGGRTLYADSVKGRFTISRDNAKNTAYLQMNNLRPEDTAVYYCAA
DLAEYSGTYSSPADSPAGYDYWGQGTQVTVSS,
or
(SEQ ID NO: 103)
QVQLVETGGGLVQAGDSLRLSCAASGRTLSFNTYAMGWFRQAPGKEREF
VASITWNGGSTSYADSVKGRFTITRDNAKNTATLRMNSLQPDDTAVYYC
AAARYYVSGTYFPANYWGQGTQVTVSS.

164. The method of any one of claims 138 to 163, wherein the VHH domain is comprised of a sequence having at least 75%, 80%, 85%, 90%, 95%, 96%, 97%, 98%, 99%, or 100% sequence identity with the sequence of
(SEQ ID NO: 93)
QVQLVESGGGLVQAGGSLKLACAAPGLTFSSYRMGWFRQAPGQEREFVA
AIDWNGRGTYYRYYADSVKGRSTISRDNAKNTMYLQMNSLKPEDTAVYY
CAAGSIDLNWYGGMDYWGQGTQVTVSS,
(SEQ ID NO: 94)
EVQVVESGGGLVQAGGSLKLACAAPGLTFSSYRMGWFRQAPGQEREFVA
AIDWNGRGTYYRYYADSVKGRSTISRDNAKNTVYLQMNSLKPEDTAVYY
CAATTVLTDPRVLNEYATWGQGTQVTVSS,
(SEQ ID NO: 95)
QLQLVESGGGLVQPGGSLRLSCAASGSIFSINVMGWYRQAPGKQRELVA
RINGGGITHYAESVKGRFTISRDNAKNTVYLQMNSLKPEDTAAYYCKAD
VFGSSGYVETYWGQGTQVTVSS,
(SEQ ID NO: 96)
EVQVVESGGGLVQAGGSLRLSCAVSGTSVSSNAMGWYRQAPGKQREWVG
FIDRIATTTIATSVKGRFAITRDNAKNTVYLQMSGLKPEDTAVYYCNHP
LTARWGQGTQVTVSS,
(SEQ ID NO: 97)
QVQLVESGGGLVQAGGSLRLSCAASGRTFSSYAMGWFRQAPGKEREFVA
AITWNGGTTYYADSVKGRFTISRDNAKNTVYLQMNSLKPEDTAVYYCAA
DPFNQGYWGQGTQVTVSS,
(SEQ ID NO: 98)
EVQLVESGGGLVQAGGSLRLSCAVSGSSVSSDAMGWYRQAPGNQRAWVA
FISGGGTTTYADSVKGRFTISRDNTKNTVYLHMNSLKPEDTAVYYCNHP
LTSRWGQGTQVTVSS,
(SEQ ID NO: 99)
EVQVVESGGGLVQAGGSLRLACVASRSIGSINVMGWYRQAPGKQRDLVA
RITGGGSTHYAESVKGRFTISRDNAKNTVYLQMNSLEPEDTAVYYCASM
VNPIITAWGTIGVREIPDYDYWGQGTQVTVSS,
(SEQ ID NO: 100)
QVQLVESGGGLVQAGGSLRLSCAVSGRTFSTYRMGWFRQAPGKERSFVA
AISWSGGSTTYADPVKGRFTISRDNAKNTVYLRMNSLKPEDTAVYYCND
QRGYWGQGTLVTVSS,
(SEQ ID NO: 101)
EVQVVESGGGLVQAGGSLRLSCAASGFTFTRYAMGWFRQAPGKERSFVA
AISWSGSSAGYGDSVKGRFTISRDNAKNTLYLQMNSLKPEDTAVYYCAA
DPFNQGYWGQGTQVTVSS,
(SEQ ID NO: 102)
EVQVVESGGGLVQAGGSLRLSCAASGRTFTTYRMGWFRQAPGKEREFVA
AIRWSGGRTLYADSVKGRFTISRDNAKNTAYLQMNNLRPEDTAVYYCAA
DLAEYSGTYSSPADSPAGYDYWGQGTQVTVSS,
or
(SEQ ID NO: 103)
QVQLVETGGGLVQAGDSLRLSCAASGRTLSFNTYAMGWFRQAPGKEREF
VASITWNGGSTSYADSVKGRFTITRDNAKNTATLRMNSLQPDDTAVYYC
AAARYYVSGTYFPANYWGQGTQVTVSS.

165. The method of any one of claims 138 to 164, wherein the agent is an antibody or fragment thereof, a peptide, a vaccine, a small molecule, a polynucleotide, a radioisotope, a toxin, an enzyme, an anticoagulant, a hormone, a cytokine, an anti-inflammatory molecule, an RNAi, an mRNA, a self-replicating RNA, an antibiotic, or an antibody-antibiotic conjugate.

166. The method of claim 165, wherein the agent is an antibiotic, an antibody or fragment thereof, a peptide or a vaccine.

167. The method of any one of claims 138 to 166, wherein the VHH domain is genetically fused or chemically conjugated to the agent.

168. The method of claim 167, wherein a linker is between the VHH domain and the agent.

169. The method of claim 168, wherein the linker is a polypeptide.

170. The method of claim 169, wherein the linker is a flexible linker comprising a sequence selected from the group consisting of EPKTPKPQPQPQLQPQPNPTTESKSPK (SEQ ID NO: 130), (EAAAK)n (SEQ ID NO: 147), (GGGGS)n (SEQ ID NO: 148) and (GGGS)n (SEQ ID NO: 149), wherein n is an integer from 1 to 20.

171. The method of any one of claims 138 to 170, wherein the VHH domain is chemically-conjugated to the agent.

172. The method of any one of claims 138 to 171, wherein the VHH domain is non-covalently bound to the agent.

173. The method of any one of claims 138 to 172, wherein the VHH domain comprises a radioisotope.

174. The method of claim 173, wherein the radioisotope is zirconium-89.

175. The method of any of claims 138 to 174, wherein the disease is lung cancer, and wherein the tissue is lung.

176. The method of any of claims 138 to 174, wherein the disease is endometrial cancer, and wherein the tissue is the uterus.

177. The method of any of claims 138 to 174, wherein the disease is colon cancer, and wherein the tissue is the colon.

178. The method of any of claims 138 to 174, wherein the disease is an inflammatory disease, and wherein the tissue is Lamina propria.

179. The method of claim 178, wherein the inflammatory disease is inflammatory bowel disease.

180. The method of claim 179, wherein the inflammatory disease is Crohn's disease or ulcerative colitis.

181. The method of any one of claims 138 to 180, wherein the diseased cell expresses an antigen, and wherein the therapeutic molecule is coupled to an antibody that specifically recognizes the antigen.

182. The method of claim 181, wherein the antigen is specific to the diseased cell.

183. A method for delivering a single domain antibody or a therapeutic molecule from an apical surface of a polymeric immunoglobulin receptor (pIgR)-expressing cell to a basolateral surface of the pIgR-expressing cell comprising contacting the pIgR-expressing cell with the single domain antibody or the therapeutic molecule, wherein the single domain antibody binds to pIgR and the therapeutic molecule comprises an agent and the single domain antibody.

184. A method for transporting a therapeutic molecule to a basolateral surface of the pIgR-expressing cell of a subject, comprising administering to the subject the therapeutic molecule comprising an agent and a single domain antibody that binds to pIgR.

185. The method of claim 184, wherein the therapeutic molecule is administered to the subject via oral delivery, buccal delivery, nasal delivery or inhalation delivery.

186. A method for transporting a therapeutic molecule to systemic circulation of a subject, comprising administering to the subject a therapeutic molecule comprising an agent and a single domain antibody that binds to pIgR, wherein the therapeutic molecule is administered to the subject via oral delivery, buccal delivery, nasal delivery or inhalation delivery.

187. A method for transporting a therapeutic molecule to Lamina propria or gastrointestinal tract of a subject, comprising administering to the subject a therapeutic molecule comprising an agent and a single domain antibody that binds to pIgR, wherein the therapeutic molecule is administered to the subject via oral delivery, buccal delivery, nasal delivery or inhalation delivery.

188. The method of any one of claims 184 to 187, wherein the therapeutic agent is transported from an apical surface of a pIgR-expressing cell to a basolateral surface of the pIgR-expressing cell in the subject.

189. The method of claim 183 or claim 188, wherein the single domain antibody or the therapeutic molecule comprising the agent and the single domain antibody is capable of being transported from the basolateral surface of the pIgR-expressing cell to the apical surface of the pIgR-expressing cell.

190. The method of any one of claims 183 to 189, wherein the pIgR-expressing cell is an epithelial cell.

191. The method of claim 190, wherein the epithelia cell is an intestinal lumen cell or an airway epithelial cell.

192. The method of any one of claims 183 to 191, wherein the agent is a diabetes medication.

193. The method of claim 192, wherein the diabetes medication is selected from a group consisting of insulin, glucagon-like-peptide-1, insulin-mimic peptides, and glucagon-like-peptide-1-mimic peptides.

194. The method of any one of claims 183 to 191, wherein the agent is a peptide or an antibody or a fragment thereof.

195. The method of claim 194, wherein the antibody or fragment thereof is selected from a group consisting of an anti-TNF-alpha antibody or a fragment thereof, an anti-IL23 antibody or a fragment thereof, an antibody or a fragment thereof that binds to a receptor of IL23, or an inhibitor of the receptor of IL23.

196. The method of any one of claims 183 to 191, wherein the agent is a vaccine.

197. The method of claim 196, wherein the vaccine is for preventing an infection selected from a group consisting of Vibrio, Cholera, Typhoid, Rotavirus, Tuberculosis, HIV, Flu, Ebola, and Sendai.

198. The method of any one of claims 183 to 197, wherein the single domain antibody binds to an extracellular domain 1, an extracellular domain 2, an extracellular domain 1-2, an extracellular domain 3, an extracellular domain 2-3, an extracellular domain 4-5, or an extracellular domain 5 of pIgR.

199. The method of any one of claims 183 to 197, wherein the single domain antibody binds to an extracellular domain 1 of pIgR.

200. The method of any one of claims 183 to 197, wherein the single domain antibody binds to an extracellular domain 2 of pIgR.

201. The method of any one of claims 183 to 197, wherein the single domain antibody binds to an extracellular domain 1-2 of pIgR.

202. The method of any one of claims 183 to 197, wherein the single domain antibody binds to an extracellular domain 3 of pIgR.

203. The method of any one of claims 183 to 197, wherein the single domain antibody binds to an extracellular domain 2-3 of pIgR.

204. The method of any one of claims 183 to 197, wherein the single domain antibody binds to an extracellular domain 4-5 of pIgR.

205. The method of any one of claims 183 to 197, wherein the single domain antibody binds to an extracellular domain 5 of pIgR.

206. The method of any one of claims 183 to 205, wherein the single domain antibody competes with IgA binding to the pIgR.

207. The method of any one of claims 183 to 205, wherein the single domain antibody promotes IgA binding to the pIgR.

208. The method of any one of claims 183 to 207, wherein the K_{D} of the binding of the single domain antibody to pIgR is from about 4 to about 525 nM.

209. The method of any one of claims 183 to 207, wherein the K_{D} of the binding of the single domain antibody to pIgR is less than about 50 nM.

210. The method of any one of claims 183 to 207, wherein the K_{D} of the binding of the single domain antibody to pIgR is from about 4 to about 34 nM.

211. The method of any one of claims 183 to 210, wherein the Tₘ of the single domain antibody is from about 53 to about 77° C.

212. The method of any one of claims 183 to 210, wherein the Tₘ of the single domain antibody is from 53.9 to 76.4° C.

213. The method of any one of claims 183 to 212, wherein pIgR is human pIgR.

214. The method of any one of claims 183 to 212, wherein pIgR is mouse pIgR.

215. The method of any one of claims 183 to 212, wherein the single domain antibody does not bind to a stalk sequence of human pIgR and/or a stalk sequence of mouse pIgR.

216. The method of any one of claims 183 to 215, wherein the single domain antibody comprises a CDR3 sequence of GSIDLNWYGGMDY (SEQ ID NO: 60), TTVLTDPRVLNEYAT (SEQ ID NO: 61), DVFGSSGYVETY (SEQ ID NO: 62), PLTAR (SEQ ID NO: 63), DPFNQGY (SEQ ID NO: 64), PLTSR (SEQ ID NO: 65), MVNPIITAWGTIGVREIPDYDY (SEQ ID NO: 66), DQRGY (SEQ ID NO: 67), QRGY (SEQ ID NO: 271), DPFNQGY (SEQ ID NO: 68), DLAEYSGTYSSPADSPAGYDY (SEQ ID NO: 69), ARYYVSGTYFPANY (SEQ ID NO: 70), GSIDLNWYGGMDY (SEQ ID NO: 71), SIDLNWYGGMD (SEQ ID NO: 272), TTVLTDPRVLNEYAT (SEQ ID NO: 72), TVLTDPRVLNEYA (SEQ ID NO: 273), DVFGSSGYVETY (SEQ ID NO: 73), VFGSSGYVET (SEQ ID NO: 274), PLTAR (SEQ ID NO: 74), LTA (SEQ ID NO: 275), DPFNQGY (SEQ ID NO: 75), PFNQG (SEQ ID NO: 276), PLTSR (SEQ ID NO: 76), LTS (SEQ ID NO: 277), MVNPIITAWGTIGVREIPDYDY (SEQ ID NO: 77), VNPIITAWGTIGVREIPDYD (SEQ ID NO: 278), DQRGY (SEQ ID NO: 78), RG (SEQ ID NO: 279), DPFNQGY (SEQ ID NO: 79), PFNQG (SEQ ID NO: 280), DLAEYSGTYSSPADSPAGYDY (SEQ ID NO: 80), LAEYSGTYSSPADSPAGYD (SEQ ID NO: 281), ARYYVSGTYFPANY (SEQ ID NO: 81), RYYVSGTYFPAN (SEQ ID NO: 282), CAAGSIDLNWYGGMDY (SEQ ID NO: 82), AAGSIDLNWYGGMDY (SEQ ID NO: 283), CAATTVLTDPRVLNEYAT (SEQ ID NO: 83), AATTVLTDPRVLNEYAT (SEQ ID NO: 284), KADVFGSSGYVETY (SEQ ID NO: 84), NHPLTAR (SEQ ID NO: 85), AADPFNQGY (SEQ ID NO: 86), NHPLTSR (SEQ ID NO: 87), ASMVNPIITAWGTIGVREIPDYDY (SEQ ID NO: 88), NDQRGY (SEQ ID NO: 89), AADPFNQGY (SEQ ID NO: 90), AADLAEYSGTYSSPADSPAGYDY (SEQ ID NO: 91), AAARYYVSGTYFPANY (SEQ ID NO: 92), GSIDLNWYGGMDY (SEQ ID NO: 214), TTVLTDPRVLNEYAT (SEQ ID NO: 215), DVFGSSGYVETY (SEQ ID NO: 216), PLTAR (SEQ ID NO: 217), DPFNQGY (SEQ ID NO: 218), PLTSR (SEQ ID NO: 219), MVNPIITAWGTIGVREIPDYDY (SEQ ID NO: 220), QRGY (SEQ ID NO: 221), DPFNQGY (SEQ ID NO: 222), DLAEYSGTYSSPADSPAGYDY (SEQ ID NO: 223), ARYYVSGTYFPANY (SEQ ID NO: 224), AAGSIDLNWYGGMD (SEQ ID NO: 225), AATTVLTDPRVLNEYA (SEQ ID NO: 226), KADVFGSSGYVET (SEQ ID NO: 227), NHPLTA (SEQ ID NO: 228), AADPFNQG (SEQ ID NO: 229), NHPLTS (SEQ ID NO: 230), ASMVNPIITAWGTIGVREIPDYD (SEQ ID NO: 231), NDQRG (SEQ ID NO: 232), AADPFNQG (SEQ ID NO: 233), AADLAEYSGTYSSPADSPAGYD (SEQ ID NO: 234), AAARYYVSGTYFPAN (SEQ ID NO: 235), GSIDLNWYGGMDY (SEQ ID NO: 236), TTVLTDPRVLNEYAT (SEQ ID NO: 237), DVFGSSGYVETY (SEQ ID NO: 238), PLTAR (SEQ ID NO: 239), DPFNQGY (SEQ ID NO: 240), PLTSR (SEQ ID NO: 241), MVNPIITAWGTIGVREIPDYDY (SEQ ID NO: 242), QRGY (SEQ ID NO: 243), DPFNQGY (SEQ ID NO: 244), DLAEYSGTYSSPADSPAGYDY (SEQ ID NO: 245), or ARYYVSGTYFPANY (SEQ ID NO: 246).

217. The method of any one of claims 183 to 216, wherein the single domain antibody comprises a CDR2 sequence of AIDWNGRGTYYRYYADSVKG (SEQ ID NO: 30), RINGGGITHYAESVKG (SEQ ID NO: 31), FIDRIATTTIATSVKG (SEQ ID NO: 32), AITWNGGTTYYADSVKG (SEQ ID NO: 33), FISGGGTTTYADSVKG (SEQ ID NO: 34), RITGGGSTHYAESVKG (SEQ ID NO: 35), AISWSGGSTTYADPVKG (SEQ ID NO: 36), AISWSGSSAGYGDSVKG (SEQ ID NO: 37), AIRWSGGRTLYADSVKG (SEQ ID NO: 38), SITWNGGSTSYADSVKG (SEQ ID NO: 39), DWNGRGTYY (SEQ ID NO: 40), WNGRGTY (SEQ ID NO: 260), NGGGI (SEQ ID NO: 41), GGG (SEQ ID NO: 261), DRIAT (SEQ ID NO: 42), RIA (SEQ ID NO: 262), TWNGGT (SEQ ID NO: 43), WNGG (SEQ ID NO: 263), SGGGT (SEQ ID NO: 44), GGG (SEQ ID NO: 264), TGGGS (SEQ ID NO: 45), GGG (SEQ ID NO: 265), SWSGGS (SEQ ID NO: 46), WSGG (SEQ ID NO: 266), SWSGSS (SEQ ID NO: 47), WSGS (SEQ ID NO: 267), RWSGGR (SEQ ID NO: 48), WSGG (SEQ ID NO: 268), TWNGGS (SEQ ID NO: 49), WNGG (SEQ ID NO: 269), IDWNGRGTYY (SEQ ID NO: 50), IDWNGRGTYYR (SEQ ID NO: 270), INGGGIT (SEQ ID NO: 51), IDRIATT (SEQ ID NO: 52), ITWNGGTT (SEQ ID NO: 53), ISGGGTT (SEQ ID NO: 54), ITGGGST (SEQ ID NO: 55), ISWSGGST (SEQ ID NO: 56), ISWSGSSA (SEQ ID NO: 57), IRWSGGRT (SEQ ID NO: 58), ITWNGGST (SEQ ID NO: 59), AIDWNGRGTYYRYYADSVKG (SEQ ID NO: 184), RINGGGITHYAESVKG (SEQ ID NO: 185), FIDRIATTTIATSVKG (SEQ ID NO: 186), AITWNGGTTYYADSVKG (SEQ ID NO: 187), FISGGGTTTYADSVKG (SEQ ID NO: 188), RITGGGSTHYAESVKG (SEQ ID NO: 189), AISWSGGSTTYADPVKG (SEQ ID NO: 190), AISWSGSSAGYGDSVKG (SEQ ID NO: 191), AIRWSGGRTLYADSVKG (SEQ ID NO: 192), SITWNGGSTSYADSVKG (SEQ ID NO: 193), FVAAIDWNGRGTYYRY (SEQ ID NO: 194), LVARINGGGITH (SEQ ID NO: 195), WVGFIDRIATTT (SEQ ID NO: 196), FVAAITWNGGTTY (SEQ ID NO: 197), WVAFISGGGTTT (SEQ ID NO: 198), LVARITGGGSTH (SEQ ID NO: 199), FVAAISWSGGSTT (SEQ ID NO: 200), FVAAISWSGSSAG (SEQ ID NO: 201), FVAAIRWSGGRTL (SEQ ID NO: 202), FVASITWNGGSTS (SEQ ID NO: 203), AIDWNGRGTYYRY (SEQ ID NO: 204), RINGGGITH (SEQ ID NO: 205), FIDRIATTT (SEQ ID NO: 206), AITWNGGTTY (SEQ ID NO: 207), FISGGGTTT (SEQ ID NO: 208), RITGGGSTH (SEQ ID NO: 209), AISWSGGSTT (SEQ ID NO: 210), AISWSGSSAG (SEQ ID NO: 211), AIRWSGGRTL (SEQ ID NO: 212), or SITWNGGSTS (SEQ ID NO: 213).

218. The method of any one of claims 183 to 217, wherein the single domain antibody comprises a CDR1 sequence of SYRMG (SEQ ID NO: 1), INVMG (SEQ ID NO: 2), SNAMG (SEQ ID NO: 3), SYAMG (SEQ ID NO: 4), SDAMG (SEQ ID NO: 5), INVMG (SEQ ID NO: 6), TYRMG (SEQ ID NO: 7), RYAMG (SEQ ID NO: 8), FTTYRMG (SEQ ID NO: 258), TYRMG (SEQ ID NO: 259), FNTYAMG (SEQ ID NO: 9), GLTFSSY (SEQ ID NO: 10), GSIFSIN (SEQ ID NO: 11), GTSVSSN (SEQ ID NO: 12), GRTFSSY (SEQ ID NO: 13), GSSVSSD (SEQ ID NO: 14), RSIGSIN (SEQ ID NO: 15), GRTFSTY (SEQ ID NO: 16), GFTFTRY (SEQ ID NO: 17), GRTFTTY (SEQ ID NO: 18), GRTLSFNTY (SEQ ID NO: 19), GLTFSSYR (SEQ ID NO: 20), GSIFSINV (SEQ ID NO: 21), GTSVSSNA (SEQ ID NO: 22), GRTFSSYA (SEQ ID NO: 23), GSSVSSDA (SEQ ID NO: 24), RSIGSINV (SEQ ID NO: 25), GRTFSTYR (SEQ ID NO: 26), GFTFTRYA (SEQ ID NO: 27), GRTFTTYR (SEQ ID NO: 28), GRTLSFNTYA (SEQ ID NO: 29), GLTFSSYRMG (SEQ ID NO: 154), GSIFSINVMG (SEQ ID NO: 155), GTSVSSNAMG (SEQ ID NO: 156), GRTFSSYAMG (SEQ ID NO: 157), GSSVSSDAMG (SEQ ID NO: 158), RSIGSINVMG (SEQ ID NO: 159), GRTFSTYRMG (SEQ ID NO: 160), GFTFTRYAMG (SEQ ID NO: 161), GRTFTTYRMG (SEQ ID NO: 162), GRTLSFNTYAMG (SEQ ID NO: 163), SSYRMG (SEQ ID NO: 164), SINVMG (SEQ ID NO: 165), SSNAMG (SEQ ID NO: 166), SSYAMG (SEQ ID NO: 167), SSDAMG (SEQ ID NO: 168), SINVMG (SEQ ID NO: 169), STYRMG (SEQ ID NO: 170), TRYAMG (SEQ ID NO: 171), TTYRMG (SEQ ID NO: 172), SFNTYAMG (SEQ ID NO: 173), GLTFSSYRMG (SEQ ID NO: 174), GSIFSINVMG (SEQ ID NO: 175), GTSVSSNAMG (SEQ ID NO: 176), GRTFSSYAMG (SEQ ID NO: 177), GSSVSSDAMG (SEQ ID NO: 178), RSIGSINVMG (SEQ ID NO: 179), GRTFSTYRMG (SEQ ID NO: 180), GFTFTRYAMG (SEQ ID NO: 181), GRTFTTYRMG (SEQ ID NO: 182), or GRTLSFNTYAMG (SEQ ID NO: 183).

219. The method of any one of claims 183 to 218, wherein the single domain antibody comprises a CDR1 sequence, a CDR2 sequence, and a CDR3 sequence of the single domain antibody selected from the group consisting of:
a) VHH1:
i) the CDR1 sequence of SYRMG (SEQ ID NO: 1), the CDR2 sequence of AIDWNGRGTYYRYYADSVKG (SEQ ID NO: 30), and the CDR3 sequence of GSIDLNWYGGMDY (SEQ ID NO: 60);
ii) the CDR1 sequence of GLTFSSY (SEQ ID NO: 10), the CDR2 sequence of DWNGRGTYY (SEQ ID NO: 40) or WNGRGTY (SEQ ID NO: 260), and the CDR3 sequence of GSIDLNWYGGMDY (SEQ ID NO: 71) or SIDLNWYGGMD (SEQ ID NO: 272);
iii) the CDR1 sequence of GLTFSSYR (SEQ ID NO: 20), the CDR2 sequence of IDWNGRGTYY (SEQ ID NO: 50) or IDWNGRGTYYR (SEQ ID NO: 270), and the CDR3 sequence of CAAGSIDLNWYGGMDY (SEQ ID NO: 82) or AAGSIDLNWYGGMDY (SEQ ID NO: 283);
iv) the CDR1 sequence of GLTFSSYRMG (SEQ ID NO: 154), the CDR 2 sequence of AIDWNGRGTYYRYYADSVKG (SEQ ID NO: 184), and the CDR3 sequence of GSIDLNWYGGMDY (SEQ ID NO: 214);
v) the CDR1 sequence of SSYRMG (SEQ ID NO: 164), the CDR2 sequence of FVAAIDWNGRGTYYRY (SEQ ID NO: 194), and the CDR3 sequence of AAGSIDLNWYGGMD (SEQ ID NO: 225); or
vi) the CDR1 sequence of GLTFSSYRMG (SEQ ID NO: 174), the CDR2 sequence of AIDWNGRGTYYRY (SEQ ID NO: 204), and the CDR3 sequence of GSIDLNWYGGMDY (SEQ ID NO: 236);
b) VHH2:
i) the CDR1 sequence of SYRMG (SEQ ID NO: 1), the CDR2 sequence of AIDWNGRGTYYRYYADSVKG (SEQ ID NO: 30), and the CDR3 sequence of TTVLTDPRVLNEYAT (SEQ ID NO: 61);
ii) the CDR1 sequence of GLTFSSY (SEQ ID NO: 10), the CDR2 sequence of DWNGRGTYY (SEQ ID NO: 40) or WNGRGTY (SEQ ID NO: 260), and the CDR3 sequence of TTVLTDPRVLNEYAT (SEQ ID NO: 72) or TVLTDPRVLNEYA (SEQ ID NO: 273);
iii) the CDR1 sequence of GLTFSSYR (SEQ ID NO: 20), the CDR2 sequence of IDWNGRGTYY (SEQ ID NO: 50) or IDWNGRGTYYR (SEQ ID NO: 270), and the CDR3 sequence of CAATTVLTDPRVLNEYAT (SEQ ID NO: 83) or AATTVLTDPRVLNEYAT (SEQ ID NO: 284);
iv) the CDR1 sequence of GLTFSSYRMG (SEQ ID NO: 154), the CDR2 sequence of AIDWNGRGTYYRYYADSVKG (SEQ ID NO: 184), and the CDR3 sequence of TTVLTDPRVLNEYAT (SEQ ID NO: 215);
v) the CDR1 sequence of SSYRMG (SEQ ID NO: 164), the CDR2 sequence of FVAAIDWNGRGTYYRY (SEQ ID NO: 194), and the CDR3 sequence of AATTVLTDPRVLNEYA (SEQ ID NO: 226); or
vi) the CDR1 sequence of GLTFSSYRMG (SEQ ID NO: 174), the CDR2 sequence of AIDWNGRGTYYRY (SEQ ID NO: 204), and the CDR3 sequence of TTVLTDPRVLNEYAT (SEQ ID NO: 237);
c) VHH3:
i) the CDR1 sequence of INVMG (SEQ ID NO: 2), the CDR2 sequence of RINGGGITHYAESVKG (SEQ ID NO: 31), and the CDR3 sequence of DVFGSSGYVETY (SEQ ID NO: 62);
ii) the CDR1 sequence of GSIFSIN (SEQ ID NO: 11), the CDR2 sequence of NGGGI (SEQ ID NO: 41) or GGG (SEQ ID NO: 261), and the CDR3 sequence of DVFGSSGYVETY (SEQ ID NO: 73) or VFGSSGYVET (SEQ ID NO: 274);
iii) the CDR1 sequence of GSIFSINV (SEQ ID NO: 21), the CDR2 sequence of INGGGIT (SEQ ID NO: 51), and the CDR3 sequence of KADVFGSSGYVETY (SEQ ID NO: 84);
iv) the CDR1 sequence of GSIFSINVMG (SEQ ID NO: 155), the CDR2 sequence of RINGGGITHYAESVKG (SEQ ID NO: 185), and the CDR3 sequence of DVFGSSGYVETY (SEQ ID NO: 216);
v) the CDR1 sequence of SINVMG (SEQ ID NO: 165), the CDR2 sequence of LVARINGGGITH (SEQ ID NO: 195), and the CDR3 sequence of KADVFGSSGYVET (SEQ ID NO: 227); or
vi) the CDR1 sequence of GSIFSINVMG (SEQ ID NO: 175), the CDR2 sequence of RINGGGITH (SEQ ID NO: 205), and the CDR3 sequence of DVFGSSGYVETY (SEQ ID NO: 238);
d) VHH4:
i) the CDR1 sequence of SNAMG (SEQ ID NO: 3), the CDR2 sequence of FIDRIATTTIATSVKG (SEQ ID NO: 32), and the CDR3 sequence of PLTAR (SEQ ID NO: 63);
ii) the CDR1 sequence of GTSVSSN (SEQ ID NO: 12), the CDR2 sequence of DRIAT (SEQ ID NO: 42) or RIA (SEQ ID NO: 262), and the CDR3 sequence of PLTAR (SEQ ID NO: 74) or LTA (SEQ ID NO: 275);
iii) the CDR1 sequence of GTSVSSNA (SEQ ID NO: 22), the CDR2 sequence of IDRIATT (SEQ ID NO: 52), and the CDR3 sequence of NHPLTAR (SEQ ID NO: 85);
iv) the CDR1 sequence of GTSVSSNAMG (SEQ ID NO: 156), the CDR2 sequence of FIDRIATTTIATSVKG (SEQ ID NO: 186), and the CDR3 sequence of PLTAR (SEQ ID NO: 217);
v) the CDR1 sequence of SSNAMG (SEQ ID NO: 166), the CDR2 sequence of WVGFIDRIATTT (SEQ ID NO: 196), and the CDR3 sequence of NHPLTA (SEQ ID NO: 228); or
vi) the CDR1 sequence of GTSVSSNAMG (SEQ ID NO: 176), the CDR2 sequence of FIDRIATTT (SEQ ID NO: 206), and the CDR3 sequence of PLTAR (SEQ ID NO: 239);
e) VHH5:
i) the CDR1 sequence of SYAMG (SEQ ID NO: 4), the CDR2 sequence of AITWNGGTTYYADSVKG (SEQ ID NO: 33), and the CDR3 sequence of DPFNQGY (SEQ ID NO: 64);
ii) the CDR1 sequence of GRTFSSY (SEQ ID NO: 13), the CDR2 sequence of TWNGGT (SEQ ID NO: 43) or WNGG (SEQ ID NO: 263), and the CDR3 sequence of DPFNQGY (SEQ ID NO: 75) or PFNQG (SEQ ID NO: 276);
iii) the CDR1 sequence of GRTFSSYA (SEQ ID NO: 23), the CDR2 sequence of ITWNGGTT (SEQ ID NO: 53), and the CDR3 sequence of AADPFNQGY (SEQ ID NO: 86);
iv) the CDR1 sequence of GRTFSSYAMG (SEQ ID NO: 157), the CDR2 sequence of AITWNGGTTYYADSVKG (SEQ ID NO: 187), and the CDR3 sequence of DPFNQGY (SEQ ID NO: 218);
v) the CDR1 sequence of SSYAMG (SEQ ID NO: 167), the CDR2 sequence of FVAAITWNGGTTY (SEQ ID NO: 197), and the CDR3 sequence of AADPFNQG (SEQ ID NO: 229); or
vi) the CDR1 sequence of GRTFSSYAMG (SEQ ID NO: 177), the CDR2 sequence of AITWNGGTTY (SEQ ID NO: 207), and the CDR3 sequence of DPFNQGY (SEQ ID NO: 240);
f) VHH6:
i) the CDR1 sequence of SDAMG (SEQ ID NO: 5), the CDR2 sequence of FISGGGTTTYADSVKG (SEQ ID NO: 34), and the CDR3 sequence of PLTSR (SEQ ID NO: 65);
ii) the CDR1 sequence of GSSVSSD (SEQ ID NO: 14), the CDR2 sequence of SGGGT (SEQ ID NO: 44) or GGG (SEQ ID NO: 264), and the CDR3 sequence of PLTSR (SEQ ID NO: 76) or LTS (SEQ ID NO: 277);
iii) the CDR1 sequence of GSSVSSDA (SEQ ID NO: 24), the CDR2 sequence of ISGGGTT (SEQ ID NO: 54), and the CDR3 sequence of NHPLTSR (SEQ ID NO: 87);
iv) the CDR1 sequence of GSSVSSDAMG (SEQ ID NO: 158), the CDR2 sequence of FISGGGTTTYADSVKG (SEQ ID NO: 188), and the CDR3 sequence of PLTSR (SEQ ID NO: 219);
v) the CDR1 sequence of SSDAMG (SEQ ID NO: 168), the CDR2 sequence of WVAFISGGGTTT (SEQ ID NO: 198), and the CDR3 sequence of NHPLTS (SEQ ID NO: 230); or
vi) the CDR1 sequence of GSSVSSDAMG (SEQ ID NO: 178), the CDR2 sequence of FISGGGTTT (SEQ ID NO: 208), and the CDR3 sequence of PLTSR (SEQ ID NO: 241);
g) VHH7:
i) the CDR1 sequence of INVMG (SEQ ID NO: 6), the CDR2 sequence of RITGGGSTHYAESVKG (SEQ ID NO: 35), and the CDR3 sequence of MVNPIITAWGTIGVREIPDYDY (SEQ ID NO: 66);
ii) the CDR1 sequence of RSIGSIN (SEQ ID NO: 15), the CDR2 sequence of TGGGS (SEQ ID NO: 45) or GGG (SEQ ID NO: 265), and the CDR3 sequence of MVNPIITAWGTIGVREIPDYDY (SEQ ID NO: 77) or VNPIITAWGTIGVREIPDYD (SEQ ID NO: 278);
iii) the CDR1 sequence of RSIGSINV (SEQ ID NO: 25), the CDR2 sequence of ITGGGST (SEQ ID NO: 55), and the CDR3 sequence of ASMVNPIITAWGTIGVREIPDYDY (SEQ ID NO: 88);
iv) the CDR1 sequence of RSIGSINVMG (SEQ ID NO: 159), the CDR2 sequence of RITGGGSTHYAESVKG (SEQ ID NO: 189), and the CDR3 sequence of MVNPIITAWGTIGVREIPDYDY (SEQ ID NO: 220);
v) the CDR1 sequence of SINVMG (SEQ ID NO: 169), the CDR2 sequence of LVARITGGGSTH (SEQ ID NO: 199), and the CDR3 sequence of ASMVNPIITAWGTIGVREIPDYD (SEQ ID NO: 231); or
vi) the CDR1 sequence of RSIGSINVMG (SEQ ID NO: 179), the CDR2 sequence of RITGGGSTH (SEQ ID NO: 209), and the CDR3 sequence of MVNPIITAWGTIGVREIPDYDY (SEQ ID NO: 242);
h) VHH9:
i) the CDR1 sequence of TYRMG (SEQ ID NO: 7), the CDR2 sequence of AISWSGGSTTYADPVKG (SEQ ID NO: 36), and the CDR3 sequence of DQRGY (SEQ ID NO: 67) or QRGY (SEQ ID NO: 271);
ii) the CDR1 sequence of GRTFSTY (SEQ ID NO: 16), the CDR2 sequence of SWSGGS (SEQ ID NO: 46) or WSGG (SEQ ID NO: 266), and the CDR3 sequence of DQRGY (SEQ ID NO: 78) or RG (SEQ ID NO: 279);
iii) the CDR1 sequence of GRTFSTYR (SEQ ID NO: 26), the CDR2 sequence of ISWSGGST (SEQ ID NO: 56), and the CDR3 sequence of NDQRGY (SEQ ID NO: 89);
iv) the CDR1 sequence of GRTFSTYRMG (SEQ ID NO: 160), the CDR2 sequence of AISWSGGSTTYADPVKG (SEQ ID NO: 190), and the CDR3 sequence of QRGY (SEQ ID NO: 221);
v) the CDR1 sequence of STYRMG (SEQ ID NO: 170), the CDR2 sequence of FVAAISWSGGSTT (SEQ ID NO: 200), and the CDR3 sequence of NDQRG (SEQ ID NO: 232); or
vi) the CDR1 sequence of GRTFSTYRMG (SEQ ID NO: 180), the CDR2 sequence of AISWSGGSTT (SEQ ID NO: 210), and the CDR3 sequence of QRGY (SEQ ID NO: 243);
i) VHH10:
i) the CDR1 sequence of RYAMG (SEQ ID NO: 8), the CDR2 sequence of AISWSGSSAGYGDSVKG (SEQ ID NO: 37), and the CDR3 sequence of DPFNQGY (SEQ ID NO: 68);
ii) the CDR1 sequence of GFTFTRY (SEQ ID NO: 17), the CDR2 sequence of SWSGSS (SEQ ID NO: 47) or WSGS (SEQ ID NO: 267), and the CDR3 sequence of DPFNQGY (SEQ ID NO: 79) or PFNQG (SEQ ID NO: 280);
iii) the CDR1 sequence of GFTFTRYA (SEQ ID NO: 27), the CDR2 sequence of ISWSGSSA (SEQ ID NO: 57), and the CDR3 sequence of AADPFNQGY (SEQ ID NO: 90);
iv) the CDR1 sequence of GFTFTRYAMG (SEQ ID NO: 161), the CDR2 sequence of AISWSGSSAGYGDSVKG (SEQ ID NO: 191), and the CDR3 sequence of DPFNQGY (SEQ ID NO: 222);
v) the CDR1 sequence of TRYAMG (SEQ ID NO: 171), the CDR2 sequence of FVAAISWSGSSAG (SEQ ID NO: 201), and the CDR3 sequence of AADPFNQG (SEQ ID NO: 233); or
vi) the CDR1 sequence of GFTFTRYAMG (SEQ ID NO: 181), the CDR2 sequence of AISWSGSSAG (SEQ ID NO: 211), and the CDR3 sequence of DPFNQGY (SEQ ID NO: 244);
j) VHH11:
i) the CDR1 sequence of FTTYRMG (SEQ ID NO: 258) or TYRMG (SEQ ID NO: 259), the CDR2 sequence of AIRWSGGRTLYADSVKG (SEQ ID NO: 38), and the CDR3 sequence of DLAEYSGTYSSPADSPAGYDY (SEQ ID NO: 69);
ii) the CDR1 sequence of GRTFTTY (SEQ ID NO: 18), the CDR2 sequence of RWSGGR (SEQ ID NO: 48) or WSGG (SEQ ID NO: 268), and the CDR3 sequence of DLAEYSGTYSSPADSPAGYDY (SEQ ID NO: 80) or LAEYSGTYSSPADSPAGYD (SEQ ID NO: 281);
iii) the CDR1 sequence of GRTFTTYR (SEQ ID NO: 28), the CDR2 sequence of IRWSGGRT (SEQ ID NO: 58), and the CDR3 sequence of AADLAEYSGTYSSPADSPAGYDY (SEQ ID NO: 91);
iv) the CDR1 sequence of GRTFTTYRMG (SEQ ID NO: 162), the CDR2 sequence of AIRWSGGRTLYADSVKG (SEQ ID NO: 192), and the CDR3 sequence of DLAEYSGTYSSPADSPAGYDY (SEQ ID NO: 223);
v) the CDR1 sequence of TTYRMG (SEQ ID NO: 172), the CDR2 sequence of FVAAIRWSGGRTL (SEQ ID NO: 202), and the CDR3 sequence of AADLAEYSGTYSSPADSPAGYD (SEQ ID NO: 234); or
vi) the CDR1 sequence of GRTFTTYRMG (SEQ ID NO: 182), the CDR2 sequence of AIRWSGGRTL (SEQ ID NO: 212), and the CDR3 sequence of DLAEYSGTYSSPADSPAGYDY (SEQ ID NO: 245); and
k) VHH12:
i) the CDR1 sequence of FNTYAMG (SEQ ID NO: 9), the CDR2 sequence of SITWNGGSTSYADSVKG (SEQ ID NO: 39), and the CDR3 sequence of ARYYVSGTYFPANY (SEQ ID NO: 70);
ii) the CDR1 sequence of GRTLSFNTY (SEQ ID NO: 19), the CDR2 sequence of TWNGGS (SEQ ID NO: 49) or WNGG (SEQ ID NO: 269), and the CDR3 sequence of ARYYVSGTYFPANY (SEQ ID NO: 81) or RYYVSGTYFPAN (SEQ ID NO: 282);
iii) the CDR1 sequence of GRTLSFNTYA (SEQ ID NO: 29), the CDR2 sequence of ITWNGGST (SEQ ID NO: 59), and the CDR3 sequence of AAARYYVSGTYFPANY (SEQ ID NO: 92);
iv) the CDR1 sequence of GRTLSFNTYAMG (SEQ ID NO: 163), the CDR2 sequence of SITWNGGSTSYADSVKG (SEQ ID NO: 193), and the CDR3 sequence of ARYYVSGTYFPANY (SEQ ID NO: 224);
v) the CDR1 sequence of SFNTYAMG (SEQ ID NO: 173), the CDR2 sequence of FVASITWNGGSTS (SEQ ID NO: 203), and the CDR3 sequence of AAARYYVSGTYFPAN (SEQ ID NO: 235); or
vi) the CDR1 sequence of GRTLSFNTYAMG (SEQ ID NO: 183), the CDR2 sequence of SITWNGGSTS (SEQ ID NO: 213), and the CDR3 sequence of ARYYVSGTYFPANY (SEQ ID NO: 246).

220. The method of any one of claims 183 to 219, wherein the single domain antibody comprises a framework derived from the framework of any of the single domain antibodies comprising the sequences of
(SEQ ID NO: 93)
QVQLVESGGGLVQAGGSLKLACAAPGLTFSSYRMGWFRQAPGQEREFVA
AIDWNGRGTYYRYYADSVKGRSTISRDNAKNTMYLQMNSLKPEDTAVYY
CAAGSIDLNWYGGMDYWGQGTQVTVSS,
(SEQ ID NO: 94)
EVQVVESGGGLVQAGGSLKLACAAPGLTFSSYRMGWFRQAPGQEREFVA
AIDWNGRGTYYRYYADSVKGRSTISRDNAKNTVYLQMNSLKPEDTAVYY
CAATTVLTDPRVLNEYATWGQGTQVTVSS,
(SEQ ID NO: 95)
QLQLVESGGGLVQPGGSLRLSCAASGSIFSINVMGWYRQAPGKQRELVA
RINGGGITHYAESVKGRFTISRDNAKNTVYLQMNSLKPEDTAAYYCKAD
VFGSSGYVETYWGQGTQVTVSS,
(SEQ ID NO: 96)
EVQVVESGGGLVQAGGSLRLSCAVSGTSVSSNAMGWYRQAPGKQREWVG
FIDRIATTTIATSVKGRFAITRDNAKNTVYLQMSGLKPEDTAVYYCNHP
LTARWGQGTQVTVSS,
(SEQ ID NO: 97)
QVQLVESGGGLVQAGGSLRLSCAASGRTFSSYAMGWFRQAPGKEREFVA
AITWNGGTTYYADSVKGRFTISRDNAKNTVYLQMNSLKPEDTAVYYCAA
DPFNQGYWGQGTQVTVSS,
(SEQ ID NO: 98)
EVQLVESGGGLVQAGGSLRLSCAVSGSSVSSDAMGWYRQAPGNQRAWVA
FISGGGTTTYADSVKGRFTISRDNTKNTVYLHMNSLKPEDTAVYYCNHP
LTSRWGQGTQVTVSS,
(SEQ ID NO: 99)
EVQVVESGGGLVQAGGSLRLACVASRSIGSINVMGWYRQAPGKQRDLVA
RITGGGSTHYAESVKGRFTISRDNAKNTVYLQMNSLEPEDTAVYYCASM
VNPIITAWGTIGVREIPDYDYWGQGTQVTVSS,
(SEQ ID NO: 100)
QVQLVESGGGLVQAGGSLRLSCAVSGRTFSTYRMGWFRQAPGKERSFVA
AISWSGGSTTYADPVKGRFTISRDNAKNTVYLRMNSLKPEDTAVYYCND
QRGYWGQGTLVTVSS,
(SEQ ID NO: 101)
EVQVVESGGGLVQAGGSLRLSCAASGFTFTRYAMGWFRQAPGKERSFVA
AISWSGSSAGYGDSVKGRFTISRDNAKNTLYLQMNSLKPEDTAVYYCAA
DPFNQGYWGQGTQVTVSS,
(SEQ ID NO: 102)
EVQVVESGGGLVQAGGSLRLSCAASGRTFTTYRMGWFRQAPGKEREFVA
AIRWSGGRTLYADSVKGRFTISRDNAKNTAYLQMNNLRPEDTAVYYCAA
DLAEYSGTYSSPADSPAGYDYWGQGTQVTVSS,
or
(SEQ ID NO: 103)
QVQLVETGGGLVQAGDSLRLSCAASGRTLSFNTYAMGWFRQAPGKEREF
VASITWNGGSTSYADSVKGRFTITRDNAKNTATLRMNSLQPDDTAVYYC
AAARYYVSGTYFPANYWGQGTQVTVSS.

221. The method of any one of claims 183 to 219, wherein the single domain antibody comprises a framework comprising sequence having at least 75%, 80%, 85%, 90%, 95%, 96%, 97%, 98%, 99%, or 100% sequence identity with the sequence of
(SEQ ID NO: 93)
QVQLVESGGGLVQAGGSLKLACAAPGLTFSSYRMGWFRQAPGQEREFVA
AIDWNGRGTYYRYYADSVKGRSTISRDNAKNTMYLQMNSLKPEDTAVYY
CAAGSIDLNWYGGMDYWGQGTQVTVSS,
(SEQ ID NO: 94)
EVQVVESGGGLVQAGGSLKLACAAPGLTFSSYRMGWFRQAPGQEREFVA
AIDWNGRGTYYRYYADSVKGRSTISRDNAKNTVYLQMNSLKPEDTAVYY
CAATTVLTDPRVLNEYATWGQGTQVTVSS,
(SEQ ID NO: 95)
QLQLVESGGGLVQPGGSLRLSCAASGSIFSINVMGWYRQAPGKQRELVA
RINGGGITHYAESVKGRFTISRDNAKNTVYLQMNSLKPEDTAAYYCKAD
VFGSSGYVETYWGQGTQVTVSS,
(SEQ ID NO: 96)
EVQVVESGGGLVQAGGSLRLSCAVSGTSVSSNAMGWYRQAPGKQREWVG
FIDRIATTTIATSVKGRFAITRDNAKNTVYLQMSGLKPEDTAVYYCNHP
LTARWGQGTQVTVSS,
(SEQ ID NO: 97)
QVQLVESGGGLVQAGGSLRLSCAASGRTFSSYAMGWFRQAPGKEREFVA
AITWNGGTTYYADSVKGRFTISRDNAKNTVYLQMNSLKPEDTAVYYCAA
DPFNQGYWGQGTQVTVSS,
(SEQ ID NO: 98)
EVQLVESGGGLVQAGGSLRLSCAVSGSSVSSDAMGWYRQAPGNQRAWVA
FISGGGTTTYADSVKGRFTISRDNTKNTVYLHMNSLKPEDTAVYYCNHP
LTSRWGQGTQVTVSS,
(SEQ ID NO: 99)
EVQVVESGGGLVQAGGSLRLACVASRSIGSINVMGWYRQAPGKQRDLVA
RITGGGSTHYAESVKGRFTISRDNAKNTVYLQMNSLEPEDTAVYYCASM
VNPIITAWGTIGVREIPDYDYWGQGTQVTVSS,
(SEQ ID NO: 100)
QVQLVESGGGLVQAGGSLRLSCAVSGRTFSTYRMGWFRQAPGKERSFVA
AISWSGGSTTYADPVKGRFTISRDNAKNTVYLRMNSLKPEDTAVYYCND
QRGYWGQGTLVTVSS,
(SEQ ID NO: 101)
EVQVVESGGGLVQAGGSLRLSCAASGFTFTRYAMGWFRQAPGKERSFVA
AISWSGSSAGYGDSVKGRFTISRDNAKNTLYLQMNSLKPEDTAVYYCAA
DPFNQGYWGQGTQVTVSS,
(SEQ ID NO: 102)
EVQVVESGGGLVQAGGSLRLSCAASGRTFTTYRMGWFRQAPGKEREFVA
AIRWSGGRTLYADSVKGRFTISRDNAKNTAYLQMNNLRPEDTAVYYCAA
DLAEYSGTYSSPADSPAGYDYWGQGTQVTVSS,
or
(SEQ ID NO: 103)
QVQLVETGGGLVQAGDSLRLSCAASGRTLSFNTYAMGWFRQAPGKEREF
VASITWNGGSTSYADSVKGRFTITRDNAKNTATLRMNSLQPDDTAVYYC
AAARYYVSGTYFPANYWGQGTQVTVSS.

222. The method of any one of claims 183 to 221, wherein the single domain antibody is comprised of a sequence having at least 75%, 80%, 85%, 90%, 95%, 96%, 97%, 98%, 99%, or 100% sequence identity with the sequence of
(SEQ ID NO: 93)
QVQLVESGGGLVQAGGSLKLACAAPGLTFSSYRMGWFRQAPGQEREFVA
AIDWNGRGTYYRYYADSVKGRSTISRDNAKNTMYLQMNSLKPEDTAVYY
CAAGSIDLNWYGGMDYWGQGTQVTVSS,
(SEQ ID NO: 94)
EVQVVESGGGLVQAGGSLKLACAAPGLTFSSYRMGWFRQAPGQEREFVA
AIDWNGRGTYYRYYADSVKGRSTISRDNAKNTVYLQMNSLKPEDTAVYY
CAATTVLTDPRVLNEYATWGQGTQVTVSS,
(SEQ ID NO: 95)
QLQLVESGGGLVQPGGSLRLSCAASGSIFSINVMGWYRQAPGKQRELVA
RINGGGITHYAESVKGRFTISRDNAKNTVYLQMNSLKPEDTAAYYCKAD
VFGSSGYVETYWGQGTQVTVSS,
(SEQ ID NO: 96)
EVQVVESGGGLVQAGGSLRLSCAVSGTSVSSNAMGWYRQAPGKQREWVG
FIDRIATTTIATSVKGRFAITRDNAKNTVYLQMSGLKPEDTAVYYCNHP
LTARWGQGTQVTVSS,
(SEQ ID NO: 97)
QVQLVESGGGLVQAGGSLRLSCAASGRTFSSYAMGWFRQAPGKEREFVA
AITWNGGTTYYADSVKGRFTISRDNAKNTVYLQMNSLKPEDTAVYYCAA
DPFNQGYWGQGTQVTVSS,
(SEQ ID NO: 98)
EVQLVESGGGLVQAGGSLRLSCAVSGSSVSSDAMGWYRQAPGNQRAWVA
FISGGGTTTYADSVKGRFTISRDNTKNTVYLHMNSLKPEDTAVYYCNHP
LTSRWGQGTQVTVSS,
(SEQ ID NO: 99)
EVQVVESGGGLVQAGGSLRLACVASRSIGSINVMGWYRQAPGKQRDLVA
RITGGGSTHYAESVKGRFTISRDNAKNTVYLQMNSLEPEDTAVYYCASM
VNPIITAWGTIGVREIPDYDYWGQGTQVTVSS,
(SEQ ID NO: 100)
QVQLVESGGGLVQAGGSLRLSCAVSGRTFSTYRMGWFRQAPGKERSFVA
AISWSGGSTTYADPVKGRFTISRDNAKNTVYLRMNSLKPEDTAVYYCND
QRGYWGQGTLVTVSS,
(SEQ ID NO: 101)
EVQVVESGGGLVQAGGSLRLSCAASGFTFTRYAMGWFRQAPGKERSFVA
AISWSGSSAGYGDSVKGRFTISRDNAKNTLYLQMNSLKPEDTAVYYCAA
DPFNQGYWGQGTQVTVSS,
(SEQ ID NO: 102)
EVQVVESGGGLVQAGGSLRLSCAASGRTFTTYRMGWFRQAPGKEREFVA
AIRWSGGRTLYADSVKGRFTISRDNAKNTAYLQMNNLRPEDTAVYYCAA
DLAEYSGTYSSPADSPAGYDYWGQGTQVTVSS,
or
(SEQ ID NO: 103)
QVQLVETGGGLVQAGDSLRLSCAASGRTLSFNTYAMGWFRQAPGKEREF
VASITWNGGSTSYADSVKGRFTITRDNAKNTATLRMNSLQPDDTAVYYC
AAARYYVSGTYFPANYWGQGTQVTVSS.

223. The method of any one of claims 183 to 222, wherein the single domain antibody is genetically fused or chemically conjugated to the agent.

224. The method of claim 223, further comprising a linker between the single domain antibody and the agent.

225. The method of claim 224, wherein the linker is a polypeptide.

226. The method of claim 225, wherein the linker is a flexible linker comprising a sequence selected from the group consisting of EPKTPKPQPQPQLQPQPNPTTESKSPK (SEQ ID NO: 130), (EAAAK)n (SEQ ID NO: 147), (GGGGS)n (SEQ ID NO: 148) and (GGGS)n (SEQ ID NO: 149), wherein n is an integer from 1 to 20.

227. The method of any one of claims 223 to 226, wherein the single domain antibody is chemically-conjugated to the agent.

228. The method of any one of claims 223 to 226, wherein the single domain antibody is non-covalently bound to the agent.

229. The method of any one of claims 183 to 228, wherein the method does not inhibit pIgR-mediated transcytosis of IgA.

230. The method of claim 229, wherein the single domain antibody comprises a CDR1 sequence of SNAMG (SEQ ID NO: 3), INVMG (SEQ ID NO: 6), TYRMG (SEQ ID NO: 7), RYAMG (SEQ ID NO: 8), FTTYRMG (SEQ ID NO: 258), TYRMG (SEQ ID NO: 259), FNTYAMG (SEQ ID NO: 9), GTSVSSN (SEQ ID NO: 12), GRTFSSY (SEQ ID NO: 13), RSIGSIN (SEQ ID NO: 15), GRTFSTY (SEQ ID NO: 16), GFTFTRY (SEQ ID NO: 17), GRTFTTY (SEQ ID NO: 18), GRTLSFNTY (SEQ ID NO: 19), GTSVSSNA (SEQ ID NO: 22), RSIGSINV (SEQ ID NO: 25), GRTFSTYR (SEQ ID NO: 26), GFTFTRYA (SEQ ID NO: 27), GRTFTTYR (SEQ ID NO: 28), GRTLSFNTYA (SEQ ID NO: 29), GTSVSSNAMG (SEQ ID NO: 156), RSIGSINVMG (SEQ ID NO: 159), GRTFSTYRMG (SEQ ID NO: 160), GFTFTRYAMG (SEQ ID NO: 161), GRTFTTYRMG (SEQ ID NO: 162), GRTLSFNTYAMG (SEQ ID NO: 163), SSNAMG (SEQ ID NO: 166), SINVMG (SEQ ID NO: 169), STYRMG (SEQ ID NO: 170), TRYAMG (SEQ ID NO: 171), TTYRMG (SEQ ID NO: 172), SFNTYAMG (SEQ ID NO: 173), GTSVSSNAMG (SEQ ID NO: 176), RSIGSINVMG (SEQ ID NO: 179), GRTFSTYRMG (SEQ ID NO: 180), GFTFTRYAMG (SEQ ID NO: 181), GRTFTTYRMG (SEQ ID NO: 182), or GRTLSFNTYAMG (SEQ ID NO: 183).

231. The method of claim 229 or claim 230, wherein the single domain antibody comprises a CDR2 sequence of FIDRIATTTIATSVKG (SEQ ID NO: 32), RITGGGSTHYAESVKG (SEQ ID NO: 35), AISWSGGSTTYADPVKG (SEQ ID NO: 36), AISWSGSSAGYGDSVKG (SEQ ID NO: 37), AIRWSGGRTLYADSVKG (SEQ ID NO: 38), SITWNGGSTSYADSVKG (SEQ ID NO: 39), DRIAT (SEQ ID NO: 42), RIA (SEQ ID NO: 262), TGGGS (SEQ ID NO: 45), GGG (SEQ ID NO: 265), SWSGGS (SEQ ID NO: 46), WSGG (SEQ ID NO: 266), SWSGSS (SEQ ID NO: 47), WSGS (SEQ ID NO: 267), RWSGGR (SEQ ID NO: 48), WSGG (SEQ ID NO: 268), TWNGGS (SEQ ID NO: 49), WNGG (SEQ ID NO: 269), IDRIATT (SEQ ID NO: 52), ITGGGST (SEQ ID NO: 55), ISWSGGST (SEQ ID NO: 56), ISWSGSSA (SEQ ID NO: 57), IRWSGGRT (SEQ ID NO: 58), ITWNGGST (SEQ ID NO: 59), FIDRIATTTIATSVKG (SEQ ID NO: 186), RITGGGSTHYAESVKG (SEQ ID NO: 189), AISWSGGSTTYADPVKG (SEQ ID NO: 190), AISWSGSSAGYGDSVKG (SEQ ID NO: 191), AIRWSGGRTLYADSVKG (SEQ ID NO: 192), SITWNGGSTSYADSVKG (SEQ ID NO: 193), WVGFIDRIATTT (SEQ ID NO: 196), LVARITGGGSTH (SEQ ID NO: 199), FVAAISWSGGSTT (SEQ ID NO: 200), FVAAISWSGSSAG (SEQ ID NO: 201), FVAAIRWSGGRTL (SEQ ID NO: 202), FVASITWNGGSTS (SEQ ID NO: 203), FIDRIATTT (SEQ ID NO: 206), RITGGGSTH (SEQ ID NO: 209), AISWSGGSTT (SEQ ID NO: 210), AISWSGSSAG (SEQ ID NO: 211), AIRWSGGRTL (SEQ ID NO: 212), or SITWNGGSTS (SEQ ID NO: 213).

232. The method of any one of claims 229 to 231, wherein the single domain antibody comprises a CDR3 sequence of PLTAR (SEQ ID NO: 63), MVNPIITAWGTIGVREIPDYDY (SEQ ID NO: 66), DQRGY (SEQ ID NO: 67), QRGY (SEQ ID NO: 271), DPFNQGY (SEQ ID NO: 68), DLAEYSGTYSSPADSPAGYDY (SEQ ID NO: 69), ARYYVSGTYFPANY (SEQ ID NO: 70), PLTAR (SEQ ID NO: 74), LTA (SEQ ID NO: 275), MVNPIITAWGTIGVREIPDYDY (SEQ ID NO: 77), VNPIITAWGTIGVREIPDYD (SEQ ID NO: 278), DQRGY (SEQ ID NO: 78), RG (SEQ ID NO: 279), DPFNQGY (SEQ ID NO: 79), PFNQG (SEQ ID NO: 280), DLAEYSGTYSSPADSPAGYDY (SEQ ID NO: 80), LAEYSGTYSSPADSPAGYD (SEQ ID NO: 281), ARYYVSGTYFPANY (SEQ ID NO: 81), RYYVSGTYFPAN (SEQ ID NO: 282), NHPLTAR (SEQ ID NO: 85), ASMVNPIITAWGTIGVREIPDYDY (SEQ ID NO: 88), NDQRGY (SEQ ID NO: 89), AADPFNQGY (SEQ ID NO: 90), AADLAEYSGTYSSPADSPAGYDY (SEQ ID NO: 91), AAARYYVSGTYFPANY (SEQ ID NO: 92), PLTAR (SEQ ID NO: 217), MVNPIITAWGTIGVREIPDYDY (SEQ ID NO: 220), QRGY (SEQ ID NO: 221), DPFNQGY (SEQ ID NO: 222), DLAEYSGTYSSPADSPAGYDY (SEQ ID NO: 223), ARYYVSGTYFPANY (SEQ ID NO: 224), NHPLTA (SEQ ID NO: 228), ASMVNPIITAWGTIGVREIPDYD (SEQ ID NO: 231), NDQRG (SEQ ID NO: 232), AADPFNQG (SEQ ID NO: 233), AADLAEYSGTYSSPADSPAGYD (SEQ ID NO: 234), AAARYYVSGTYFPAN (SEQ ID NO: 235), PLTAR (SEQ ID NO: 239), MVNPIITAWGTIGVREIPDYDY (SEQ ID NO: 242), QRGY (SEQ ID NO: 243), DPFNQGY (SEQ ID NO: 244), DLAEYSGTYSSPADSPAGYDY (SEQ ID NO: 245), or ARYYVSGTYFPANY (SEQ ID NO: 246).

233. A process for providing a molecule to a subject, comprising administering to the subject the molecule comprising an agent and a single domain antibody that binds to polymeric immunoglobulin receptor (pIgR), wherein the molecule is administered to the subject via oral delivery, buccal delivery, nasal delivery or inhalation delivery.

234. The process of claim 233, wherein the molecule is capable of being provided to a basolateral surface of a pIgR-expressing cell from an apical surface of the pIgR-expressing cell in the subject.

235. The process of claim 233 or claim 234, wherein the molecule is capable of being provided to an apical surface of the pIgR-expressing cell from a basolateral surface of a pIgR-expressing cell in the subject.

236. The process of any one of claims 233 to 235, wherein the pIgR-expressing cell is an epithelial cell.

237. The process of claim 236, wherein the epithelia cell is an intestinal lumen cell or an airway epithelial cell.

238. The process of any one of claims 233 to 237, wherein the agent is a diabetes medication.

239. The process of claim 238, wherein the diabetes medication is selected from a group consisting of insulin, glucagon-like-peptide-1, insulin-mimic peptides, and glucagon-like-peptide-1-mimic peptides.

240. The process of any one of claims 233 to 237, wherein the agent is a peptide or an antibody or a fragment thereof.

241. The process of claim 240, wherein the antibody or fragment thereof is selected from a group consisting of an anti-TNF-alpha antibody or a fragment thereof, an anti-IL23 antibody or a fragment thereof, and an antibody that binds to a receptor of IL23 or a fragment thereof.

242. The process of any one of claims 233 to 237, wherein the agent is a vaccine.

243. The process of claim 242, wherein the vaccine is for preventing an infection selected from a group consisting of Vibrio, Cholera, Typhoid, Rotavirus, Tuberculosis, HIV, Flu, Ebola, and Sendai.

244. The process of any one of claims 233 to 243, wherein the single domain antibody binds to an extracellular domain 1, an extracellular domain 2, an extracellular domain 1-2, an extracellular domain 3, an extracellular domain 2-3, an extracellular domain 4-5, or an extracellular domain 5 of pIgR.

245. The process of any one of claims 233 to 243, wherein the single domain antibody binds to an extracellular domain 1 of pIgR.

246. The process of any one of claims 233 to 243, wherein the single domain antibody binds to an extracellular domain 2 of pIgR.

247. The process of any one of claims 233 to 243, wherein the single domain antibody binds to an extracellular domain 1-2 of pIgR.

248. The process of any one of claims 233 to 243, wherein the single domain antibody binds to an extracellular domain 3 of pIgR.

249. The process of any one of claims 233 to 243, wherein the single domain antibody binds to an extracellular domain 2-3 of pIgR.

250. The process of any one of claims 233 to 243, wherein the single domain antibody binds to an extracellular domain 4-5 of pIgR.

251. The process of any one of claims 233 to 243, wherein the single domain antibody binds to an extracellular domain 5 of pIgR.

252. The process of any one of claims 233 to 251, wherein the single domain antibody competes with IgA binding to the pIgR.

253. The process of any one of claims 233 to 251, wherein the single domain antibody promotes IgA binding to the pIgR.

254. The process of any one of claims 233 to 253, wherein the K_{D} of the binding of the single domain antibody to pIgR is from about 4 to about 525 nM.

255. The process of any one of claims 233 to 253, wherein the K_{D} of the binding of the single domain antibody to pIgR is less than about 50 nM.

256. The process of any one of claims 233 to 253, wherein the K_{D} of the binding of the single domain antibody to pIgR is from about 4 to about 34 nM.

257. The process of any one of claims 233 to 256, wherein the Tₘ of the single domain antibody is from about 53 to about 77° C.

258. The process of any one of claims 233 to 256, wherein the Tₘ of the single domain antibody is from 53.9 to 76.4° C.

259. The process of any one of claims 233 to 258, wherein pIgR is human pIgR.

260. The process of any one of claims 233 to 258, wherein pIgR is mouse pIgR.

261. The process of any one of claims 233 to 258, wherein the single domain antibody does not bind to a stalk sequence of human pIgR and/or a stalk sequence of mouse pIgR.

262. The process of any one of claims 233 to 261, wherein the single domain antibody comprises a CDR3 sequence of GSIDLNWYGGMDY (SEQ ID NO: 60), TTVLTDPRVLNEYAT (SEQ ID NO: 61), DVFGSSGYVETY (SEQ ID NO: 62), PLTAR (SEQ ID NO: 63), DPFNQGY (SEQ ID NO: 64), PLTSR (SEQ ID NO: 65), MVNPIITAWGTIGVREIPDYDY (SEQ ID NO: 66), DQRGY (SEQ ID NO: 67), QRGY (SEQ ID NO: 271), DPFNQGY (SEQ ID NO: 68), DLAEYSGTYSSPADSPAGYDY (SEQ ID NO: 69), ARYYVSGTYFPANY (SEQ ID NO: 70), GSIDLNWYGGMDY (SEQ ID NO: 71), SIDLNWYGGMD (SEQ ID NO: 272), TTVLTDPRVLNEYAT (SEQ ID NO: 72), TVLTDPRVLNEYA (SEQ ID NO: 273), DVFGSSGYVETY (SEQ ID NO: 73), VFGSSGYVET (SEQ ID NO: 274), PLTAR (SEQ ID NO: 74), LTA (SEQ ID NO: 275), DPFNQGY (SEQ ID NO: 75), PFNQG (SEQ ID NO: 276), PLTSR (SEQ ID NO: 76), LTS (SEQ ID NO: 277), MVNPIITAWGTIGVREIPDYDY (SEQ ID NO: 77), VNPIITAWGTIGVREIPDYD (SEQ ID NO: 278), DQRGY (SEQ ID NO: 78), RG (SEQ ID NO: 279), DPFNQGY (SEQ ID NO: 79), PFNQG (SEQ ID NO: 280), DLAEYSGTYSSPADSPAGYDY (SEQ ID NO: 80), LAEYSGTYSSPADSPAGYD (SEQ ID NO: 281), ARYYVSGTYFPANY (SEQ ID NO: 81), RYYVSGTYFPAN (SEQ ID NO: 282), CAAGSIDLNWYGGMDY (SEQ ID NO: 82), AAGSIDLNWYGGMDY (SEQ ID NO: 283), CAATTVLTDPRVLNEYAT (SEQ ID NO: 83), AATTVLTDPRVLNEYAT (SEQ ID NO: 284), KADVFGSSGYVETY (SEQ ID NO: 84), NHPLTAR (SEQ ID NO: 85), AADPFNQGY (SEQ ID NO: 86), NHPLTSR (SEQ ID NO: 87), ASMVNPIITAWGTIGVREIPDYDY (SEQ ID NO: 88), NDQRGY (SEQ ID NO: 89), AADPFNQGY (SEQ ID NO: 90), AADLAEYSGTYSSPADSPAGYDY (SEQ ID NO: 91), AAARYYVSGTYFPANY (SEQ ID NO: 92), GSIDLNWYGGMDY (SEQ ID NO: 214), TTVLTDPRVLNEYAT (SEQ ID NO: 215), DVFGSSGYVETY (SEQ ID NO: 216), PLTAR (SEQ ID NO: 217), DPFNQGY (SEQ ID NO: 218), PLTSR (SEQ ID NO: 219), MVNPIITAWGTIGVREIPDYDY (SEQ ID NO: 220), QRGY (SEQ ID NO: 221), DPFNQGY (SEQ ID NO: 222), DLAEYSGTYSSPADSPAGYDY (SEQ ID NO: 223), ARYYVSGTYFPANY (SEQ ID NO: 224), AAGSIDLNWYGGMD (SEQ ID NO: 225), AATTVLTDPRVLNEYA (SEQ ID NO: 226), KADVFGSSGYVET (SEQ ID NO: 227), NHPLTA (SEQ ID NO: 228), AADPFNQG (SEQ ID NO: 229), NHPLTS (SEQ ID NO: 230), ASMVNPIITAWGTIGVREIPDYD (SEQ ID NO: 231), NDQRG (SEQ ID NO: 232), AADPFNQG (SEQ ID NO: 233), AADLAEYSGTYSSPADSPAGYD (SEQ ID NO: 234), AAARYYVSGTYFPAN (SEQ ID NO: 235), GSIDLNWYGGMDY (SEQ ID NO: 236), TTVLTDPRVLNEYAT (SEQ ID NO: 237), DVFGSSGYVETY (SEQ ID NO: 238), PLTAR (SEQ ID NO: 239), DPFNQGY (SEQ ID NO: 240), PLTSR (SEQ ID NO: 241), MVNPIITAWGTIGVREIPDYDY (SEQ ID NO: 242), QRGY (SEQ ID NO: 243), DPFNQGY (SEQ ID NO: 244), DLAEYSGTYSSPADSPAGYDY (SEQ ID NO: 245), or ARYYVSGTYFPANY (SEQ ID NO: 246).

263. The process of any one of claims 233 to 262, wherein the single domain antibody comprises a CDR2 sequence of AIDWNGRGTYYRYYADSVKG (SEQ ID NO: 30), RINGGGITHYAESVKG (SEQ ID NO: 31), FIDRIATTTIATSVKG (SEQ ID NO: 32), AITWNGGTTYYADSVKG (SEQ ID NO: 33), FISGGGTTTYADSVKG (SEQ ID NO: 34), RITGGGSTHYAESVKG (SEQ ID NO: 35), AISWSGGSTTYADPVKG (SEQ ID NO: 36), AISWSGSSAGYGDSVKG (SEQ ID NO: 37), AIRWSGGRTLYADSVKG (SEQ ID NO: 38), SITWNGGSTSYADSVKG (SEQ ID NO: 39), DWNGRGTYY (SEQ ID NO: 40), WNGRGTY (SEQ ID NO: 260), NGGGI (SEQ ID NO: 41), GGG (SEQ ID NO: 261), DRIAT (SEQ ID NO: 42), RIA (SEQ ID NO: 262), TWNGGT (SEQ ID NO: 43), WNGG (SEQ ID NO: 263), SGGGT (SEQ ID NO: 44), GGG (SEQ ID NO: 264), TGGGS (SEQ ID NO: 45), GGG (SEQ ID NO: 265), SWSGGS (SEQ ID NO: 46), WSGG (SEQ ID NO: 266), SWSGSS (SEQ ID NO: 47), WSGS (SEQ ID NO: 267), RWSGGR (SEQ ID NO: 48), WSGG (SEQ ID NO: 268), TWNGGS (SEQ ID NO: 49), WNGG (SEQ ID NO: 269), IDWNGRGTYY (SEQ ID NO: 50), IDWNGRGTYYR (SEQ ID NO: 270), INGGGIT (SEQ ID NO: 51), IDRIATT (SEQ ID NO: 52), ITWNGGTT (SEQ ID NO: 53), ISGGGTT (SEQ ID NO: 54), ITGGGST (SEQ ID NO: 55), ISWSGGST (SEQ ID NO: 56), ISWSGSSA (SEQ ID NO: 57), IRWSGGRT (SEQ ID NO: 58), ITWNGGST (SEQ ID NO: 59), AIDWNGRGTYYRYYADSVKG (SEQ ID NO: 184), RINGGGITHYAESVKG (SEQ ID NO: 185), FIDRIATTTIATSVKG (SEQ ID NO: 186), AITWNGGTTYYADSVKG (SEQ ID NO: 187), FISGGGTTTYADSVKG (SEQ ID NO: 188), RITGGGSTHYAESVKG (SEQ ID NO: 189), AISWSGGSTTYADPVKG (SEQ ID NO: 190), AISWSGSSAGYGDSVKG (SEQ ID NO: 191), AIRWSGGRTLYADSVKG (SEQ ID NO: 192), SITWNGGSTSYADSVKG (SEQ ID NO: 193), FVAAIDWNGRGTYYRY (SEQ ID NO: 194), LVARINGGGITH (SEQ ID NO: 195), WVGFIDRIATTT (SEQ ID NO: 196), FVAAITWNGGTTY (SEQ ID NO: 197), WVAFISGGGTTT (SEQ ID NO: 198), LVARITGGGSTH (SEQ ID NO: 199), FVAAISWSGGSTT (SEQ ID NO: 200), FVAAISWSGSSAG (SEQ ID NO: 201), FVAAIRWSGGRTL (SEQ ID NO: 202), FVASITWNGGSTS (SEQ ID NO: 203), AIDWNGRGTYYRY (SEQ ID NO: 204), RINGGGITH (SEQ ID NO: 205), FIDRIATTT (SEQ ID NO: 206), AITWNGGTTY (SEQ ID NO: 207), FISGGGTTT (SEQ ID NO: 208), RITGGGSTH (SEQ ID NO: 209), AISWSGGSTT (SEQ ID NO: 210), AISWSGSSAG (SEQ ID NO: 211), AIRWSGGRTL (SEQ ID NO: 212), or SITWNGGSTS (SEQ ID NO: 213).

264. The process of any one of claims 233 to 263, wherein the single domain antibody comprises a CDR1 sequence of SYRMG (SEQ ID NO: 1), INVMG (SEQ ID NO: 2), SNAMG (SEQ ID NO: 3), SYAMG (SEQ ID NO: 4), SDAMG (SEQ ID NO: 5), INVMG (SEQ ID NO: 6), TYRMG (SEQ ID NO: 7), RYAMG (SEQ ID NO: 8), FTTYRMG (SEQ ID NO: 258), TYRMG (SEQ ID NO: 259), FNTYAMG (SEQ ID NO: 9), GLTFSSY (SEQ ID NO: 10), GSIFSIN (SEQ ID NO: 11), GTSVSSN (SEQ ID NO: 12), GRTFSSY (SEQ ID NO: 13), GSSVSSD (SEQ ID NO: 14), RSIGSIN (SEQ ID NO: 15), GRTFSTY (SEQ ID NO: 16), GFTFTRY (SEQ ID NO: 17), GRTFTTY (SEQ ID NO: 18), GRTLSFNTY (SEQ ID NO: 19), GLTFSSYR (SEQ ID NO: 20), GSIFSINV (SEQ ID NO: 21), GTSVSSNA (SEQ ID NO: 22), GRTFSSYA (SEQ ID NO: 23), GSSVSSDA (SEQ ID NO: 24), RSIGSINV (SEQ ID NO: 25), GRTFSTYR (SEQ ID NO: 26), GFTFTRYA (SEQ ID NO: 27), GRTFTTYR (SEQ ID NO: 28), GRTLSFNTYA (SEQ ID NO: 29), GLTFSSYRMG (SEQ ID NO: 154), GSIFSINVMG (SEQ ID NO: 155), GTSVSSNAMG (SEQ ID NO: 156), GRTFSSYAMG (SEQ ID NO: 157), GSSVSSDAMG (SEQ ID NO: 158), RSIGSINVMG (SEQ ID NO: 159), GRTFSTYRMG (SEQ ID NO: 160), GFTFTRYAMG (SEQ ID NO: 161), GRTFTTYRMG (SEQ ID NO: 162), GRTLSFNTYAMG (SEQ ID NO: 163), SSYRMG (SEQ ID NO: 164), SINVMG (SEQ ID NO: 165), SSNAMG (SEQ ID NO: 166), SSYAMG (SEQ ID NO: 167), SSDAMG (SEQ ID NO: 168), SINVMG (SEQ ID NO: 169), STYRMG (SEQ ID NO: 170), TRYAMG (SEQ ID NO: 171), TTYRMG (SEQ ID NO: 172), SFNTYAMG (SEQ ID NO: 173), GLTFSSYRMG (SEQ ID NO: 174), GSIFSINVMG (SEQ ID NO: 175), GTSVSSNAMG (SEQ ID NO: 176), GRTFSSYAMG (SEQ ID NO: 177), GSSVSSDAMG (SEQ ID NO: 178), RSIGSINVMG (SEQ ID NO: 179), GRTFSTYRMG (SEQ ID NO: 180), GFTFTRYAMG (SEQ ID NO: 181), GRTFTTYRMG (SEQ ID NO: 182), or GRTLSFNTYAMG (SEQ ID NO: 183).

265. The process of any one of claims 233 to 264, wherein the single domain antibody comprises a CDR1 sequence, a CDR2 sequence, and a CDR3 sequence of the single domain antibody selected from the group consisting of:
a) VHH1:
i) the CDR1 sequence of SYRMG (SEQ ID NO: 1), the CDR2 sequence of AIDWNGRGTYYRYYADSVKG (SEQ ID NO: 30), and the CDR3 sequence of GSIDLNWYGGMDY (SEQ ID NO: 60);
ii) the CDR1 sequence of GLTFSSY (SEQ ID NO: 10), the CDR2 sequence of DWNGRGTYY (SEQ ID NO: 40) or WNGRGTY (SEQ ID NO: 260), and the CDR3 sequence of GSIDLNWYGGMDY (SEQ ID NO: 71) or SIDLNWYGGMD (SEQ ID NO: 272);
iii) the CDR1 sequence of GLTFSSYR (SEQ ID NO: 20), the CDR2 sequence of IDWNGRGTYY (SEQ ID NO: 50) or IDWNGRGTYYR (SEQ ID NO: 270), and the CDR3 sequence of CAAGSIDLNWYGGMDY (SEQ ID NO: 82) or AAGSIDLNWYGGMDY (SEQ ID NO: 283);
iv) the CDR1 sequence of GLTFSSYRMG (SEQ ID NO: 154), the CDR 2 sequence of AIDWNGRGTYYRYYADSVKG (SEQ ID NO: 184), and the CDR3 sequence of GSIDLNWYGGMDY (SEQ ID NO: 214);
v) the CDR1 sequence of SSYRMG (SEQ ID NO: 164), the CDR2 sequence of FVAAIDWNGRGTYYRY (SEQ ID NO: 194), and the CDR3 sequence of AAGSIDLNWYGGMD (SEQ ID NO: 225); or
vi) the CDR1 sequence of GLTFSSYRMG (SEQ ID NO: 174), the CDR2 sequence of AIDWNGRGTYYRY (SEQ ID NO: 204), and the CDR3 sequence of GSIDLNWYGGMDY (SEQ ID NO: 236);
b) VHH2:
i) the CDR1 sequence of SYRMG (SEQ ID NO: 1), the CDR2 sequence of AIDWNGRGTYYRYYADSVKG (SEQ ID NO: 30), and the CDR3 sequence of TTVLTDPRVLNEYAT (SEQ ID NO: 61);
ii) the CDR1 sequence of GLTFSSY (SEQ ID NO: 10), the CDR2 sequence of DWNGRGTYY (SEQ ID NO: 40) or WNGRGTY (SEQ ID NO: 260), and the CDR3 sequence of TTVLTDPRVLNEYAT (SEQ ID NO: 72) or TVLTDPRVLNEYA (SEQ ID NO: 273);
iii) the CDR1 sequence of GLTFSSYR (SEQ ID NO: 20), the CDR2 sequence of IDWNGRGTYY (SEQ ID NO: 50) or IDWNGRGTYYR (SEQ ID NO: 270), and the CDR3 sequence of CAATTVLTDPRVLNEYAT (SEQ ID NO: 83) or AATTVLTDPRVLNEYAT (SEQ ID NO: 284);
iv) the CDR1 sequence of GLTFSSYRMG (SEQ ID NO: 154), the CDR2 sequence of AIDWNGRGTYYRYYADSVKG (SEQ ID NO: 184), and the CDR3 sequence of TTVLTDPRVLNEYAT (SEQ ID NO: 215);
v) the CDR1 sequence of SSYRMG (SEQ ID NO: 164), the CDR2 sequence of FVAAIDWNGRGTYYRY (SEQ ID NO: 194), and the CDR3 sequence of AATTVLTDPRVLNEYA (SEQ ID NO: 226); or
vi) the CDR1 sequence of GLTFSSYRMG (SEQ ID NO: 174), the CDR2 sequence of AIDWNGRGTYYRY (SEQ ID NO: 204), and the CDR3 sequence of TTVLTDPRVLNEYAT (SEQ ID NO: 237);
c) VHH3:
i) the CDR1 sequence of INVMG (SEQ ID NO: 2), the CDR2 sequence of RINGGGITHYAESVKG (SEQ ID NO: 31), and the CDR3 sequence of DVFGSSGYVETY (SEQ ID NO: 62);
ii) the CDR1 sequence of GSIFSIN (SEQ ID NO: 11), the CDR2 sequence of NGGGI (SEQ ID NO: 41) or GGG (SEQ ID NO: 261), and the CDR3 sequence of DVFGSSGYVETY (SEQ ID NO: 73) or VFGSSGYVET (SEQ ID NO: 274);
iii) the CDR1 sequence of GSIFSINV (SEQ ID NO: 21), the CDR2 sequence of INGGGIT (SEQ ID NO: 51), and the CDR3 sequence of KADVFGSSGYVETY (SEQ ID NO: 84);
iv) the CDR1 sequence of GSIFSINVMG (SEQ ID NO: 155), the CDR2 sequence of RINGGGITHYAESVKG (SEQ ID NO: 185), and the CDR3 sequence of DVFGSSGYVETY (SEQ ID NO: 216);
v) the CDR1 sequence of SINVMG (SEQ ID NO: 165), the CDR2 sequence of LVARINGGGITH (SEQ ID NO: 195), and the CDR3 sequence of KADVFGSSGYVET (SEQ ID NO: 227); or
vi) the CDR1 sequence of GSIFSINVMG (SEQ ID NO: 175), the CDR2 sequence of RINGGGITH (SEQ ID NO: 205), and the CDR3 sequence of DVFGSSGYVETY (SEQ ID NO: 238);
d) VHH4:
i) the CDR1 sequence of SNAMG (SEQ ID NO: 3), the CDR2 sequence of FIDRIATTTIATSVKG (SEQ ID NO: 32), and the CDR3 sequence of PLTAR (SEQ ID NO: 63);
ii) the CDR1 sequence of GTSVSSN (SEQ ID NO: 12), the CDR2 sequence of DRIAT (SEQ ID NO: 42) or RIA (SEQ ID NO: 262), and the CDR3 sequence of PLTAR (SEQ ID NO: 74) or LTA (SEQ ID NO: 275);
iii) the CDR1 sequence of GTSVSSNA (SEQ ID NO: 22), the CDR2 sequence of IDRIATT (SEQ ID NO: 52), and the CDR3 sequence of NHPLTAR (SEQ ID NO: 85);
iv) the CDR1 sequence of GTSVSSNAMG (SEQ ID NO: 156), the CDR2 sequence of FIDRIATTTIATSVKG (SEQ ID NO: 186), and the CDR3 sequence of PLTAR (SEQ ID NO: 217);
v) the CDR1 sequence of SSNAMG (SEQ ID NO: 166), the CDR2 sequence of WVGFIDRIATTT (SEQ ID NO: 196), and the CDR3 sequence of NHPLTA (SEQ ID NO: 228); or
vi) the CDR1 sequence of GTSVSSNAMG (SEQ ID NO: 176), the CDR2 sequence of FIDRIATTT (SEQ ID NO: 206), and the CDR3 sequence of PLTAR (SEQ ID NO: 239);
e) VHH5:
i) the CDR1 sequence of SYAMG (SEQ ID NO: 4), the CDR2 sequence of AITWNGGTTYYADSVKG (SEQ ID NO: 33), and the CDR3 sequence of DPFNQGY (SEQ ID NO: 64);
ii) the CDR1 sequence of GRTFSSY (SEQ ID NO: 13), the CDR2 sequence of TWNGGT (SEQ ID NO: 43) or WNGG (SEQ ID NO: 263), and the CDR3 sequence of DPFNQGY (SEQ ID NO: 75) or PFNQG (SEQ ID NO: 276);
iii) the CDR1 sequence of GRTFSSYA (SEQ ID NO: 23), the CDR2 sequence of ITWNGGTT (SEQ ID NO: 53), and the CDR3 sequence of AADPFNQGY (SEQ ID NO: 86);
iv) the CDR1 sequence of GRTFSSYAMG (SEQ ID NO: 157), the CDR2 sequence of AITWNGGTTYYADSVKG (SEQ ID NO: 187), and the CDR3 sequence of DPFNQGY (SEQ ID NO: 218);
v) the CDR1 sequence of SSYAMG (SEQ ID NO: 167), the CDR2 sequence of FVAAITWNGGTTY (SEQ ID NO: 197), and the CDR3 sequence of AADPFNQG (SEQ ID NO: 229); or
vi) the CDR1 sequence of GRTFSSYAMG (SEQ ID NO: 177), the CDR2 sequence of AITWNGGTTY (SEQ ID NO: 207), and the CDR3 sequence of DPFNQGY (SEQ ID NO: 240);
f) VHH6:
i) the CDR1 sequence of SDAMG (SEQ ID NO: 5), the CDR2 sequence of FISGGGTTTYADSVKG (SEQ ID NO: 34), and the CDR3 sequence of PLTSR (SEQ ID NO: 65);
ii) the CDR1 sequence of GSSVSSD (SEQ ID NO: 14), the CDR2 sequence of SGGGT (SEQ ID NO: 44) or GGG (SEQ ID NO: 264), and the CDR3 sequence of PLTSR (SEQ ID NO: 76) or LTS (SEQ ID NO: 277);
iii) the CDR1 sequence of GSSVSSDA (SEQ ID NO: 24), the CDR2 sequence of ISGGGTT (SEQ ID NO: 54), and the CDR3 sequence of NHPLTSR (SEQ ID NO: 87);
iv) the CDR1 sequence of GSSVSSDAMG (SEQ ID NO: 158), the CDR2 sequence of FISGGGTTTYADSVKG (SEQ ID NO: 188), and the CDR3 sequence of PLTSR (SEQ ID NO: 219);
v) the CDR1 sequence of SSDAMG (SEQ ID NO: 168), the CDR2 sequence of WVAFISGGGTTT (SEQ ID NO: 198), and the CDR3 sequence of NHPLTS (SEQ ID NO: 230); or
vi) the CDR1 sequence of GSSVSSDAMG (SEQ ID NO: 178), the CDR2 sequence of FISGGGTTT (SEQ ID NO: 208), and the CDR3 sequence of PLTSR (SEQ ID NO: 241);
g) VHH7:
i) the CDR1 sequence of INVMG (SEQ ID NO: 6), the CDR2 sequence of RITGGGSTHYAESVKG (SEQ ID NO: 35), and the CDR3 sequence of MVNPIITAWGTIGVREIPDYDY (SEQ ID NO: 66);
ii) the CDR1 sequence of RSIGSIN (SEQ ID NO: 15), the CDR2 sequence of TGGGS (SEQ ID NO: 45) or GGG (SEQ ID NO: 265), and the CDR3 sequence of MVNPIITAWGTIGVREIPDYDY (SEQ ID NO: 77) or VNPIITAWGTIGVREIPDYD (SEQ ID NO: 278);
iii) the CDR1 sequence of RSIGSINV (SEQ ID NO: 25), the CDR2 sequence of ITGGGST (SEQ ID NO: 55), and the CDR3 sequence of ASMVNPIITAWGTIGVREIPDYDY (SEQ ID NO: 88);
iv) the CDR1 sequence of RSIGSINVMG (SEQ ID NO: 159), the CDR2 sequence of RITGGGSTHYAESVKG (SEQ ID NO: 189), and the CDR3 sequence of MVNPIITAWGTIGVREIPDYDY (SEQ ID NO: 220);
v) the CDR1 sequence of SINVMG (SEQ ID NO: 169), the CDR2 sequence of LVARITGGGSTH (SEQ ID NO: 199), and the CDR3 sequence of ASMVNPIITAWGTIGVREIPDYD (SEQ ID NO: 231); or
vi) the CDR1 sequence of RSIGSINVMG (SEQ ID NO: 179), the CDR2 sequence of RITGGGSTH (SEQ ID NO: 209), and the CDR3 sequence of MVNPIITAWGTIGVREIPDYDY (SEQ ID NO: 242);
h) VHH9:
i) the CDR1 sequence of TYRMG (SEQ ID NO: 7), the CDR2 sequence of AISWSGGSTTYADPVKG (SEQ ID NO: 36), and the CDR3 sequence of DQRGY (SEQ ID NO: 67) or QRGY (SEQ ID NO: 271);
ii) the CDR1 sequence of GRTFSTY (SEQ ID NO: 16), the CDR2 sequence of SWSGGS (SEQ ID NO: 46) or WSGG (SEQ ID NO: 266), and the CDR3 sequence of DQRGY (SEQ ID NO: 78) or RG (SEQ ID NO: 279);
iii) the CDR1 sequence of GRTFSTYR (SEQ ID NO: 26), the CDR2 sequence of ISWSGGST (SEQ ID NO: 56), and the CDR3 sequence of NDQRGY (SEQ ID NO: 89);
iv) the CDR1 sequence of GRTFSTYRMG (SEQ ID NO: 160), the CDR2 sequence of AISWSGGSTTYADPVKG (SEQ ID NO: 190), and the CDR3 sequence of QRGY (SEQ ID NO: 221);
v) the CDR1 sequence of STYRMG (SEQ ID NO: 170), the CDR2 sequence of FVAAISWSGGSTT (SEQ ID NO: 200), and the CDR3 sequence of NDQRG (SEQ ID NO: 232); or
vi) the CDR1 sequence of GRTFSTYRMG (SEQ ID NO: 180), the CDR2 sequence of AISWSGGSTT (SEQ ID NO: 210), and the CDR3 sequence of QRGY (SEQ ID NO: 243);
i) VHH10:
i) the CDR1 sequence of RYAMG (SEQ ID NO: 8), the CDR2 sequence of AISWSGSSAGYGDSVKG (SEQ ID NO: 37), and the CDR3 sequence of DPFNQGY (SEQ ID NO: 68);
ii) the CDR1 sequence of GFTFTRY (SEQ ID NO: 17), the CDR2 sequence of SWSGSS (SEQ ID NO: 47) or WSGS (SEQ ID NO: 267), and the CDR3 sequence of DPFNQGY (SEQ ID NO: 79) or PFNQG (SEQ ID NO: 280);
iii) the CDR1 sequence of GFTFTRYA (SEQ ID NO: 27), the CDR2 sequence of ISWSGSSA (SEQ ID NO: 57), and the CDR3 sequence of AADPFNQGY (SEQ ID NO: 90);
iv) the CDR1 sequence of GFTFTRYAMG (SEQ ID NO: 161), the CDR2 sequence of AISWSGSSAGYGDSVKG (SEQ ID NO: 191), and the CDR3 sequence of DPFNQGY (SEQ ID NO: 222);
v) the CDR1 sequence of TRYAMG (SEQ ID NO: 171), the CDR2 sequence of FVAAISWSGSSAG (SEQ ID NO: 201), and the CDR3 sequence of AADPFNQG (SEQ ID NO: 233); or
vi) the CDR1 sequence of GFTFTRYAMG (SEQ ID NO: 181), the CDR2 sequence of AISWSGSSAG (SEQ ID NO: 211), and the CDR3 sequence of DPFNQGY (SEQ ID NO: 244);
j) VHH11:
i) the CDR1 sequence of FTTYRMG (SEQ ID NO: 258) or TYRMG (SEQ ID NO: 259), the CDR2 sequence of AIRWSGGRTLYADSVKG (SEQ ID NO: 38), and the CDR3 sequence of DLAEYSGTYSSPADSPAGYDY (SEQ ID NO: 69);
ii) the CDR1 sequence of GRTFTTY (SEQ ID NO: 18), the CDR2 sequence of RWSGGR (SEQ ID NO: 48) or WSGG (SEQ ID NO: 268), and the CDR3 sequence of DLAEYSGTYSSPADSPAGYDY (SEQ ID NO: 80) or LAEYSGTYSSPADSPAGYD (SEQ ID NO: 281);
iii) the CDR1 sequence of GRTFTTYR (SEQ ID NO: 28), the CDR2 sequence of IRWSGGRT (SEQ ID NO: 58), and the CDR3 sequence of AADLAEYSGTYSSPADSPAGYDY (SEQ ID NO: 91);
iv) the CDR1 sequence of GRTFTTYRMG (SEQ ID NO: 162), the CDR2 sequence of AIRWSGGRTLYADSVKG (SEQ ID NO: 192), and the CDR3 sequence of DLAEYSGTYSSPADSPAGYDY (SEQ ID NO: 223);
v) the CDR1 sequence of TTYRMG (SEQ ID NO: 172), the CDR2 sequence of FVAAIRWSGGRTL (SEQ ID NO: 202), and the CDR3 sequence of AADLAEYSGTYSSPADSPAGYD (SEQ ID NO: 234); or
vi) the CDR1 sequence of GRTFTTYRMG (SEQ ID NO: 182), the CDR2 sequence of AIRWSGGRTL (SEQ ID NO: 212), and the CDR3 sequence of DLAEYSGTYSSPADSPAGYDY (SEQ ID NO: 245); and
k) VHH12:
i) the CDR1 sequence of FNTYAMG (SEQ ID NO: 9), the CDR2 sequence of SITWNGGSTSYADSVKG (SEQ ID NO: 39), and the CDR3 sequence of ARYYVSGTYFPANY (SEQ ID NO: 70);
ii) the CDR1 sequence of GRTLSFNTY (SEQ ID NO: 19), the CDR2 sequence of TWNGGS (SEQ ID NO: 49) or WNGG (SEQ ID NO: 269), and the CDR3 sequence of ARYYVSGTYFPANY (SEQ ID NO: 81) or RYYVSGTYFPAN (SEQ ID NO: 282);
iii) the CDR1 sequence of GRTLSFNTYA (SEQ ID NO: 29), the CDR2 sequence of ITWNGGST (SEQ ID NO: 59), and the CDR3 sequence of AAARYYVSGTYFPANY (SEQ ID NO: 92);
iv) the CDR1 sequence of GRTLSFNTYAMG (SEQ ID NO: 163), the CDR2 sequence of SITWNGGSTSYADSVKG (SEQ ID NO: 193), and the CDR3 sequence of ARYYVSGTYFPANY (SEQ ID NO: 224);
v) the CDR1 sequence of SFNTYAMG (SEQ ID NO: 173), the CDR2 sequence of FVASITWNGGSTS (SEQ ID NO: 203), and the CDR3 sequence of AAARYYVSGTYFPAN (SEQ ID NO: 235); or
vi) the CDR1 sequence of GRTLSFNTYAMG (SEQ ID NO: 183), the CDR2 sequence of SITWNGGSTS (SEQ ID NO: 213), and the CDR3 sequence of ARYYVSGTYFPANY (SEQ ID NO: 246).

266. The process of any one of claims 233 to 265, wherein the single domain antibody comprises a framework derived from the framework of any of the single domain antibodies comprising the sequences of
(SEQ ID NO: 93)
QVQLVESGGGLVQAGGSLKLACAAPGLTFSSYRMGWFRQAPGQEREFVA
AIDWNGRGTYYRYYADSVKGRSTISRDNAKNTMYLQMNSLKPEDTAVYY
CAAGSIDLNWYGGMDYWGQGTQVTVSS,
(SEQ ID NO: 94)
EVQVVESGGGLVQAGGSLKLACAAPGLTFSSYRMGWFRQAPGQEREFVA
AIDWNGRGTYYRYYADSVKGRSTISRDNAKNTVYLQMNSLKPEDTAVYY
CAATTVLTDPRVLNEYATWGQGTQVTVSS,
(SEQ ID NO: 95)
QLQLVESGGGLVQPGGSLRLSCAASGSIFSINVMGWYRQAPGKQRELVA
RINGGGITHYAESVKGRFTISRDNAKNTVYLQMNSLKPEDTAAYYCKAD
VFGSSGYVETYWGQGTQVTVSS,
(SEQ ID NO: 96)
EVQVVESGGGLVQAGGSLRLSCAVSGTSVSSNAMGWYRQAPGKQREWVG
FIDRIATTTIATSVKGRFAITRDNAKNTVYLQMSGLKPEDTAVYYCNHP
LTARWGQGTQVTVSS,
(SEQ ID NO: 97)
QVQLVESGGGLVQAGGSLRLSCAASGRTFSSYAMGWFRQAPGKEREFVA
AITWNGGTTYYADSVKGRFTISRDNAKNTVYLQMNSLKPEDTAVYYCAA
DPFNQGYWGQGTQVTVSS,
(SEQ ID NO: 98)
EVQLVESGGGLVQAGGSLRLSCAVSGSSVSSDAMGWYRQAPGNQRAWVA
FISGGGTTTYADSVKGRFTISRDNTKNTVYLHMNSLKPEDTAVYYCNHP
LTSRWGQGTQVTVSS,
(SEQ ID NO: 99)
EVQVVESGGGLVQAGGSLRLACVASRSIGSINVMGWYRQAPGKQRDLVA
RITGGGSTHYAESVKGRFTISRDNAKNTVYLQMNSLEPEDTAVYYCASM
VNPIITAWGTIGVREIPDYDYWGQGTQVTVSS,
(SEQ ID NO: 100)
QVQLVESGGGLVQAGGSLRLSCAVSGRTFSTYRMGWFRQAPGKERSFVA
AISWSGGSTTYADPVKGRFTISRDNAKNTVYLRMNSLKPEDTAVYYCND
QRGYWGQGTLVTVSS,
(SEQ ID NO: 101)
EVQVVESGGGLVQAGGSLRLSCAASGFTFTRYAMGWFRQAPGKERSFVA
AISWSGSSAGYGDSVKGRFTISRDNAKNTLYLQMNSLKPEDTAVYYCAA
DPFNQGYWGQGTQVTVSS,
(SEQ ID NO: 102)
EVQVVESGGGLVQAGGSLRLSCAASGRTFTTYRMGWFRQAPGKEREFVA
AIRWSGGRTLYADSVKGRFTISRDNAKNTAYLQMNNLRPEDTAVYYCAA
DLAEYSGTYSSPADSPAGYDYWGQGTQVTVSS,
or
(SEQ ID NO: 103)
QVQLVETGGGLVQAGDSLRLSCAASGRTLSFNTYAMGWFRQAPGKEREF
VASITWNGGSTSYADSVKGRFTITRDNAKNTATLRMNSLQPDDTAVYYC
AAARYYVSGTYFPANYWGQGTQVTVSS.

267. The process of any one of claims 233 to 265, wherein the single domain antibody comprises a framework comprising sequence having at least 75%, 80%, 85%, 90%, 95%, 96%, 97%, 98%, 99%, or 100% sequence identity with the sequence of
(SEQ ID NO: 93)
QVQLVESGGGLVQAGGSLKLACAAPGLTFSSYRMGWFRQAPGQEREFVA
AIDWNGRGTYYRYYADSVKGRSTISRDNAKNTMYLQMNSLKPEDTAVYY
CAAGSIDLNWYGGMDYWGQGTQVTVSS,
(SEQ ID NO: 94)
EVQVVESGGGLVQAGGSLKLACAAPGLTFSSYRMGWFRQAPGQEREFVA
AIDWNGRGTYYRYYADSVKGRSTISRDNAKNTVYLQMNSLKPEDTAVYY
CAATTVLTDPRVLNEYATWGQGTQVTVSS,
(SEQ ID NO: 95)
QLQLVESGGGLVQPGGSLRLSCAASGSIFSINVMGWYRQAPGKQRELVA
RINGGGITHYAESVKGRFTISRDNAKNTVYLQMNSLKPEDTAAYYCKAD
VFGSSGYVETYWGQGTQVTVSS,
(SEQ ID NO: 96)
EVQVVESGGGLVQAGGSLRLSCAVSGTSVSSNAMGWYRQAPGKQREWVG
FIDRIATTTIATSVKGRFAITRDNAKNTVYLQMSGLKPEDTAVYYCNHP
LTARWGQGTQVTVSS,
(SEQ ID NO: 97)
QVQLVESGGGLVQAGGSLRLSCAASGRTFSSYAMGWFRQAPGKEREFVA
AITWNGGTTYYADSVKGRFTISRDNAKNTVYLQMNSLKPEDTAVYYCAA
DPFNQGYWGQGTQVTVSS,
(SEQ ID NO: 98)
EVQLVESGGGLVQAGGSLRLSCAVSGSSVSSDAMGWYRQAPGNQRAWVA
FISGGGTTTYADSVKGRFTISRDNTKNTVYLHMNSLKPEDTAVYYCNHP
LTSRWGQGTQVTVSS,
(SEQ ID NO: 99)
EVQVVESGGGLVQAGGSLRLACVASRSIGSINVMGWYRQAPGKQRDLVA
RITGGGSTHYAESVKGRFTISRDNAKNTVYLQMNSLEPEDTAVYYCASM
VNPIITAWGTIGVREIPDYDYWGQGTQVTVSS,
(SEQ ID NO: 100)
QVQLVESGGGLVQAGGSLRLSCAVSGRTFSTYRMGWFRQAPGKERSFVA
AISWSGGSTTYADPVKGRFTISRDNAKNTVYLRMNSLKPEDTAVYYCND
QRGYWGQGTLVTVSS,
(SEQ ID NO: 101)
EVQVVESGGGLVQAGGSLRLSCAASGFTFTRYAMGWFRQAPGKERSFVA
AISWSGSSAGYGDSVKGRFTISRDNAKNTLYLQMNSLKPEDTAVYYCAA
DPFNQGYWGQGTQVTVSS,
(SEQ ID NO: 102)
EVQVVESGGGLVQAGGSLRLSCAASGRTFTTYRMGWFRQAPGKEREFVA
AIRWSGGRTLYADSVKGRFTISRDNAKNTAYLQMNNLRPEDTAVYYCAA
DLAEYSGTYSSPADSPAGYDYWGQGTQVTVSS,
or
(SEQ ID NO: 103)
QVQLVETGGGLVQAGDSLRLSCAASGRTLSFNTYAMGWFRQAPGKEREF
VASITWNGGSTSYADSVKGRFTITRDNAKNTATLRMNSLQPDDTAVYYC
AAARYYVSGTYFPANYWGQGTQVTVSS.

268. The process of any one of claims 233 to 267, wherein the single domain antibody is comprised of a sequence having at least 75%, 80%, 85%, 90%, 95%, 96%, 97%, 98%, 99%, or 100% sequence identity with the sequence of
(SEQ ID NO: 93)
QVQLVESGGGLVQAGGSLKLACAAPGLTFSSYRMGWFRQAPGQEREFVA
AIDWNGRGTYYRYYADSVKGRSTISRDNAKNTMYLQMNSLKPEDTAVYY
CAAGSIDLNWYGGMDYWGQGTQVTVSS,
(SEQ ID NO: 94)
EVQVVESGGGLVQAGGSLKLACAAPGLTFSSYRMGWFRQAPGQEREFVA
AIDWNGRGTYYRYYADSVKGRSTISRDNAKNTVYLQMNSLKPEDTAVYY
CAATTVLTDPRVLNEYATWGQGTQVTVSS,
(SEQ ID NO: 95)
QLQLVESGGGLVQPGGSLRLSCAASGSIFSINVMGWYRQAPGKQRELVA
RINGGGITHYAESVKGRFTISRDNAKNTVYLQMNSLKPEDTAAYYCKAD
VFGSSGYVETYWGQGTQVTVSS,
(SEQ ID NO: 96)
EVQVVESGGGLVQAGGSLRLSCAVSGTSVSSNAMGWYRQAPGKQREWVG
FIDRIATTTIATSVKGRFAITRDNAKNTVYLQMSGLKPEDTAVYYCNHP
LTARWGQGTQVTVSS,
(SEQ ID NO: 97)
QVQLVESGGGLVQAGGSLRLSCAASGRTFSSYAMGWFRQAPGKEREFVA
AITWNGGTTYYADSVKGRFTISRDNAKNTVYLQMNSLKPEDTAVYYCAA
DPFNQGYWGQGTQVTVSS,
(SEQ ID NO: 98)
EVQLVESGGGLVQAGGSLRLSCAVSGSSVSSDAMGWYRQAPGNQRAWVA
FISGGGTTTYADSVKGRFTISRDNTKNTVYLHMNSLKPEDTAVYYCNHP
LTSRWGQGTQVTVSS,
(SEQ ID NO: 99)
EVQVVESGGGLVQAGGSLRLACVASRSIGSINVMGWYRQAPGKQRDLVA
RITGGGSTHYAESVKGRFTISRDNAKNTVYLQMNSLEPEDTAVYYCASM
VNPIITAWGTIGVREIPDYDYWGQGTQVTVSS,
(SEQ ID NO: 100)
QVQLVESGGGLVQAGGSLRLSCAVSGRTFSTYRMGWFRQAPGKERSFVA
AISWSGGSTTYADPVKGRFTISRDNAKNTVYLRMNSLKPEDTAVYYCND
QRGYWGQGTLVTVSS,
(SEQ ID NO: 101)
EVQVVESGGGLVQAGGSLRLSCAASGFTFTRYAMGWFRQAPGKERSFVA
AISWSGSSAGYGDSVKGRFTISRDNAKNTLYLQMNSLKPEDTAVYYCAA
DPFNQGYWGQGTQVTVSS,
(SEQ ID NO: 102)
EVQVVESGGGLVQAGGSLRLSCAASGRTFTTYRMGWFRQAPGKEREFVA
AIRWSGGRTLYADSVKGRFTISRDNAKNTAYLQMNNLRPEDTAVYYCAA
DLAEYSGTYSSPADSPAGYDYWGQGTQVTVSS,
or
(SEQ ID NO: 103)
QVQLVETGGGLVQAGDSLRLSCAASGRTLSFNTYAMGWFRQAPGKEREF
VASITWNGGSTSYADSVKGRFTITRDNAKNTATLRMNSLQPDDTAVYYC
AAARYYVSGTYFPANYWGQGTQVTVSS.

269. The process of any one of claims 233 to 268, wherein the single domain antibody is genetically fused or chemically conjugated to the agent.

270. The process of claim 269, further comprising a linker between the single domain antibody and the agent.

271. The process of claim 270, wherein the linker is a polypeptide.

272. The process of claim 271, wherein the linker is a flexible linker comprising a sequence selected from the group consisting of EPKTPKPQPQPQLQPQPNPTTESKSPK (SEQ ID NO: 130), (EAAAK)n (SEQ ID NO: 147), (GGGGS)n (SEQ ID NO: 148) and (GGGS)n (SEQ ID NO: 149), wherein n is an integer from 1 to 20.

273. The process of any one of claims 269 to 272, wherein the single domain antibody is chemically-conjugated to the agent.

274. The process of any one of claims 269 to 272, wherein the single domain antibody is non-covalently bound to the agent.

275. The process of any one of claims 233 to 274, wherein the process does not inhibit pIgR-mediated transcytosis of IgA.

276. The process of claim 275, wherein the single domain antibody comprises a CDR1 sequence of SNAMG (SEQ ID NO: 3), INVMG (SEQ ID NO: 6), TYRMG (SEQ ID NO: 7), RYAMG (SEQ ID NO: 8), FTTYRMG (SEQ ID NO: 258), TYRMG (SEQ ID NO: 259), FNTYAMG (SEQ ID NO: 9), GTSVSSN (SEQ ID NO: 12), GRTFSSY (SEQ ID NO: 13), RSIGSIN (SEQ ID NO: 15), GRTFSTY (SEQ ID NO: 16), GFTFTRY (SEQ ID NO: 17), GRTFTTY (SEQ ID NO: 18), GRTLSFNTY (SEQ ID NO: 19), GTSVSSNA (SEQ ID NO: 22), RSIGSINV (SEQ ID NO: 25), GRTFSTYR (SEQ ID NO: 26), GFTFTRYA (SEQ ID NO: 27), GRTFTTYR (SEQ ID NO: 28), GRTLSFNTYA (SEQ ID NO: 29), GTSVSSNAMG (SEQ ID NO: 156), RSIGSINVMG (SEQ ID NO: 159), GRTFSTYRMG (SEQ ID NO: 160), GFTFTRYAMG (SEQ ID NO: 161), GRTFTTYRMG (SEQ ID NO: 162), GRTLSFNTYAMG (SEQ ID NO: 163), SSNAMG (SEQ ID NO: 166), SINVMG (SEQ ID NO: 169), STYRMG (SEQ ID NO: 170), TRYAMG (SEQ ID NO: 171), TTYRMG (SEQ ID NO: 172), SFNTYAMG (SEQ ID NO: 173), GTSVSSNAMG (SEQ ID NO: 176), RSIGSINVMG (SEQ ID NO: 179), GRTFSTYRMG (SEQ ID NO: 180), GFTFTRYAMG (SEQ ID NO: 181), GRTFTTYRMG (SEQ ID NO: 182), or GRTLSFNTYAMG (SEQ ID NO: 183).

277. The process of claim 275 or claim 276, wherein the single domain antibody comprises a CDR2 sequence of FIDRIATTTIATSVKG (SEQ ID NO: 32), RITGGGSTHYAESVKG (SEQ ID NO: 35), AISWSGGSTTYADPVKG (SEQ ID NO: 36), AISWSGSSAGYGDSVKG (SEQ ID NO: 37), AIRWSGGRTLYADSVKG (SEQ ID NO: 38), SITWNGGSTSYADSVKG (SEQ ID NO: 39), DRIAT (SEQ ID NO: 42), RIA (SEQ ID NO: 262), TGGGS (SEQ ID NO: 45), GGG (SEQ ID NO: 265), SWSGGS (SEQ ID NO: 46), WSGG (SEQ ID NO: 266), SWSGSS (SEQ ID NO: 47), WSGS (SEQ ID NO: 267), RWSGGR (SEQ ID NO: 48), WSGG (SEQ ID NO: 268), TWNGGS (SEQ ID NO: 49), WNGG (SEQ ID NO: 269), IDRIATT (SEQ ID NO: 52), ITGGGST (SEQ ID NO: 55), ISWSGGST (SEQ ID NO: 56), ISWSGSSA (SEQ ID NO: 57), IRWSGGRT (SEQ ID NO: 58), ITWNGGST (SEQ ID NO: 59), FIDRIATTTIATSVKG (SEQ ID NO: 186), RITGGGSTHYAESVKG (SEQ ID NO: 189), AISWSGGSTTYADPVKG (SEQ ID NO: 190), AISWSGSSAGYGDSVKG (SEQ ID NO: 191), AIRWSGGRTLYADSVKG (SEQ ID NO: 192), SITWNGGSTSYADSVKG (SEQ ID NO: 193), WVGFIDRIATTT (SEQ ID NO: 196), LVARITGGGSTH (SEQ ID NO: 199), FVAAISWSGGSTT (SEQ ID NO: 200), FVAAISWSGSSAG (SEQ ID NO: 201), FVAAIRWSGGRTL (SEQ ID NO: 202), FVASITWNGGSTS (SEQ ID NO: 203), FIDRIATTT (SEQ ID NO: 206), RITGGGSTH (SEQ ID NO: 209), AISWSGGSTT (SEQ ID NO: 210), AISWSGSSAG (SEQ ID NO: 211), AIRWSGGRTL (SEQ ID NO: 212), or SITWNGGSTS (SEQ ID NO: 213).

278. The process of any one of claims 275 to 277, wherein the single domain antibody comprises a CDR3 sequence of PLTAR (SEQ ID NO: 63), MVNPIITAWGTIGVREIPDYDY (SEQ ID NO: 66), DQRGY (SEQ ID NO: 67), QRGY (SEQ ID NO: 271), DPFNQGY (SEQ ID NO: 68), DLAEYSGTYSSPADSPAGYDY (SEQ ID NO: 69), ARYYVSGTYFPANY (SEQ ID NO: 70), PLTAR (SEQ ID NO: 74), LTA (SEQ ID NO: 275), MVNPIITAWGTIGVREIPDYDY (SEQ ID NO: 77), VNPIITAWGTIGVREIPDYD (SEQ ID NO: 278), DQRGY (SEQ ID NO: 78), RG (SEQ ID NO: 279), DPFNQGY (SEQ ID NO: 79), PFNQG (SEQ ID NO: 280), DLAEYSGTYSSPADSPAGYDY (SEQ ID NO: 80), LAEYSGTYSSPADSPAGYD (SEQ ID NO: 281), ARYYVSGTYFPANY (SEQ ID NO: 81), RYYVSGTYFPAN (SEQ ID NO: 282), NHPLTAR (SEQ ID NO: 85), ASMVNPIITAWGTIGVREIPDYDY (SEQ ID NO: 88), NDQRGY (SEQ ID NO: 89), AADPFNQGY (SEQ ID NO: 90), AADLAEYSGTYSSPADSPAGYDY (SEQ ID NO: 91), AAARYYVSGTYFPANY (SEQ ID NO: 92), PLTAR (SEQ ID NO: 217), MVNPIITAWGTIGVREIPDYDY (SEQ ID NO: 220), QRGY (SEQ ID NO: 221), DPFNQGY (SEQ ID NO: 222), DLAEYSGTYSSPADSPAGYDY (SEQ ID NO: 223), ARYYVSGTYFPANY (SEQ ID NO: 224), NHPLTA (SEQ ID NO: 228), ASMVNPIITAWGTIGVREIPDYD (SEQ ID NO: 231), NDQRG (SEQ ID NO: 232), AADPFNQG (SEQ ID NO: 233), AADLAEYSGTYSSPADSPAGYD (SEQ ID NO: 234), AAARYYVSGTYFPAN (SEQ ID NO: 235), PLTAR (SEQ ID NO: 239), MVNPIITAWGTIGVREIPDYDY (SEQ ID NO: 242), QRGY (SEQ ID NO: 243), DPFNQGY (SEQ ID NO: 244), DLAEYSGTYSSPADSPAGYDY (SEQ ID NO: 245), or ARYYVSGTYFPANY (SEQ ID NO: 246).

279. A process comprising steps for providing a molecule to a subject.

280. The process of claim 279, wherein the molecule comprises an agent and a single domain antibody that binds to pIgR.

281. The process of claim 280, wherein the agent is an antibody or fragment thereof, a peptide, a vaccine, a small molecule, a polynucleotide, a radioisotope, a toxin, an enzyme, an anticoagulant, a hormone, a cytokine, an anti-inflammatory molecule, an RNAi, an antibiotic, or an antibody-antibiotic conjugate.

282. The process of any one of claims 279 to 281, wherein the agent is an antibody or fragment thereof, a peptide, or a vaccine.

283. The process of any one of claims 280 to 282, wherein the single domain antibody is genetically fused or chemically conjugated to the agent.

284. A system for providing a molecule to Lamina propria of a subject, comprising a molecule suitable for administering to the subject, the molecule comprising an agent and a single domain antibody that binds to pIgR, wherein the molecule is administered to the subject via oral delivery, buccal delivery, nasal delivery or inhalation delivery, or a combination thereof.

285. The system of claim 284, wherein the agent is a diabetes medication.

286. The system of claim 285, wherein the diabetes medication is selected from a group consisting of insulin, glucagon-like-peptide-1, insulin-mimic peptides, and glucagon-like-peptide-1-mimic peptides.

287. The system of claim 284, wherein the agent is a peptide or an antibody or a fragment thereof.

288. The system of claim 287, wherein the antibody or fragment thereof is selected from a group consisting of an anti-TNF-alpha antibody or a fragment thereof, an anti-IL23 antibody or a fragment thereof, and an antibody that binds to a receptor of IL23 or a fragment thereof.

289. The system of claim 284, wherein the agent is a vaccine.

290. The system of claim 289, wherein the vaccine is for preventing an infection selected from a group consisting of Vibrio, Cholera, Typhoid, Rotavirus, Tuberculosis, HIV, Flu, Ebola, and Sendai.

291. The system of any one of claims 284 to 290, wherein the single domain antibody binds to an extracellular domain 1, an extracellular domain 2, an extracellular domain 1-2, an extracellular domain 3, an extracellular domain 2-3, an extracellular domain 4-5, or an extracellular domain 5 of pIgR.

292. The system of any one of claims 284 to 290, wherein the single domain antibody binds to an extracellular domain 1 of pIgR.

293. The system of any one of claims 284 to 290, wherein the single domain antibody binds to an extracellular domain 2 of pIgR.

294. The system of any one of claims 284 to 290, wherein the single domain antibody binds to an extracellular domain 1-2 of pIgR.

295. The system of any one of claims 284 to 290, wherein the single domain antibody binds to an extracellular domain 3 of pIgR.

296. The system of any one of claims 284 to 290, wherein the single domain antibody binds to an extracellular domain 2-3 of pIgR.

297. The system of any one of claims 284 to 290, wherein the single domain antibody binds to an extracellular domain 4-5 of pIgR.

298. The system of any one of claims 284 to 290, wherein the single domain antibody binds to an extracellular domain 5 of pIgR.

299. The system of any one of claims 284 to 298, wherein the single domain antibody competes with IgA binding to the pIgR.

300. The system of any one of claims 284 to 298, wherein the single domain antibody promotes IgA binding to the pIgR.

301. The system of any one of claims 284 to 300, wherein the K_{D} of the binding of the single domain antibody to pIgR is from about 4 to about 525 nM.

302. The system of any one of claims 284 to 300, wherein the K_{D} of the binding of the single domain antibody to pIgR is less than about 50 nM.

303. The system of any one of claims 284 to 300, wherein the K_{D} of the binding of the single domain antibody to pIgR is from about 4 to about 34 nM.

304. The system of any one of claims 284 to 303, wherein the Tₘ of the single domain antibody is from about 53 to about 77° C.

305. The system of any one of claims 284 to 303, wherein the Tₘ of the single domain antibody is from 53.9 to 76.4° C.

306. The system of any one of claims 284 to 305, wherein pIgR is human pIgR.

307. The system of any one of claims 284 to 305, wherein pIgR is mouse pIgR.

308. The system of any one of claims 284 to 305, wherein the single domain antibody does not bind to a stalk sequence of human pIgR and/or a stalk sequence of mouse pIgR.

309. The system of any one of claims 284 to 308, wherein the single domain antibody comprises a CDR3 sequence of GSIDLNWYGGMDY (SEQ ID NO: 60), TTVLTDPRVLNEYAT (SEQ ID NO: 61), DVFGSSGYVETY (SEQ ID NO: 62), PLTAR (SEQ ID NO: 63), DPFNQGY (SEQ ID NO: 64), PLTSR (SEQ ID NO: 65), MVNPIITAWGTIGVREIPDYDY (SEQ ID NO: 66), DQRGY (SEQ ID NO: 67), QRGY (SEQ ID NO: 271), DPFNQGY (SEQ ID NO: 68), DLAEYSGTYSSPADSPAGYDY (SEQ ID NO: 69), ARYYVSGTYFPANY (SEQ ID NO: 70), GSIDLNWYGGMDY (SEQ ID NO: 71), SIDLNWYGGMD (SEQ ID NO: 272), TTVLTDPRVLNEYAT (SEQ ID NO: 72), TVLTDPRVLNEYA (SEQ ID NO: 273), DVFGSSGYVETY (SEQ ID NO: 73), VFGSSGYVET (SEQ ID NO: 274), PLTAR (SEQ ID NO: 74), LTA (SEQ ID NO: 275), DPFNQGY (SEQ ID NO: 75), PFNQG (SEQ ID NO: 276), PLTSR (SEQ ID NO: 76), LTS (SEQ ID NO: 277), MVNPIITAWGTIGVREIPDYDY (SEQ ID NO: 77), VNPIITAWGTIGVREIPDYD (SEQ ID NO: 278), DQRGY (SEQ ID NO: 78), RG (SEQ ID NO: 279), DPFNQGY (SEQ ID NO: 79), PFNQG (SEQ ID NO: 280), DLAEYSGTYSSPADSPAGYDY (SEQ ID NO: 80), LAEYSGTYSSPADSPAGYD (SEQ ID NO: 281), ARYYVSGTYFPANY (SEQ ID NO: 81), RYYVSGTYFPAN (SEQ ID NO: 282), CAAGSIDLNWYGGMDY (SEQ ID NO: 82), AAGSIDLNWYGGMDY (SEQ ID NO: 283), CAATTVLTDPRVLNEYAT (SEQ ID NO: 83), AATTVLTDPRVLNEYAT (SEQ ID NO: 284), KADVFGSSGYVETY (SEQ ID NO: 84), NHPLTAR (SEQ ID NO: 85), AADPFNQGY (SEQ ID NO: 86), NHPLTSR (SEQ ID NO: 87), ASMVNPIITAWGTIGVREIPDYDY (SEQ ID NO: 88), NDQRGY (SEQ ID NO: 89), AADPFNQGY (SEQ ID NO: 90), AADLAEYSGTYSSPADSPAGYDY (SEQ ID NO: 91), AAARYYVSGTYFPANY (SEQ ID NO: 92), GSIDLNWYGGMDY (SEQ ID NO: 214), TTVLTDPRVLNEYAT (SEQ ID NO: 215), DVFGSSGYVETY (SEQ ID NO: 216), PLTAR (SEQ ID NO: 217), DPFNQGY (SEQ ID NO: 218), PLTSR (SEQ ID NO: 219), MVNPIITAWGTIGVREIPDYDY (SEQ ID NO: 220), QRGY (SEQ ID NO: 221), DPFNQGY (SEQ ID NO: 222), DLAEYSGTYSSPADSPAGYDY (SEQ ID NO: 223), ARYYVSGTYFPANY (SEQ ID NO: 224), AAGSIDLNWYGGMD (SEQ ID NO: 225), AATTVLTDPRVLNEYA (SEQ ID NO: 226), KADVFGSSGYVET (SEQ ID NO: 227), NHPLTA (SEQ ID NO: 228), AADPFNQG (SEQ ID NO: 229), NHPLTS (SEQ ID NO: 230), ASMVNPIITAWGTIGVREIPDYD (SEQ ID NO: 231), NDQRG (SEQ ID NO: 232), AADPFNQG (SEQ ID NO: 233), AADLAEYSGTYSSPADSPAGYD (SEQ ID NO: 234), AAARYYVSGTYFPAN (SEQ ID NO: 235), GSIDLNWYGGMDY (SEQ ID NO: 236), TTVLTDPRVLNEYAT (SEQ ID NO: 237), DVFGSSGYVETY (SEQ ID NO: 238), PLTAR (SEQ ID NO: 239), DPFNQGY (SEQ ID NO: 240), PLTSR (SEQ ID NO: 241), MVNPIITAWGTIGVREIPDYDY (SEQ ID NO: 242), QRGY (SEQ ID NO: 243), DPFNQGY (SEQ ID NO: 244), DLAEYSGTYSSPADSPAGYDY (SEQ ID NO: 245), or ARYYVSGTYFPANY (SEQ ID NO: 246).

310. The system of any one of claims 284 to 309, wherein the single domain antibody comprises a CDR2 sequence of AIDWNGRGTYYRYYADSVKG (SEQ ID NO: 30), RINGGGITHYAESVKG (SEQ ID NO: 31), FIDRIATTTIATSVKG (SEQ ID NO: 32), AITWNGGTTYYADSVKG (SEQ ID NO: 33), FISGGGTTTYADSVKG (SEQ ID NO: 34), RITGGGSTHYAESVKG (SEQ ID NO: 35), AISWSGGSTTYADPVKG (SEQ ID NO: 36), AISWSGSSAGYGDSVKG (SEQ ID NO: 37), AIRWSGGRTLYADSVKG (SEQ ID NO: 38), SITWNGGSTSYADSVKG (SEQ ID NO: 39), DWNGRGTYY (SEQ ID NO: 40), WNGRGTY (SEQ ID NO: 260), NGGGI (SEQ ID NO: 41), GGG (SEQ ID NO: 261), DRIAT (SEQ ID NO: 42), RIA (SEQ ID NO: 262), TWNGGT (SEQ ID NO: 43), WNGG (SEQ ID NO: 263), SGGGT (SEQ ID NO: 44), GGG (SEQ ID NO: 264), TGGGS (SEQ ID NO: 45), GGG (SEQ ID NO: 265), SWSGGS (SEQ ID NO: 46), WSGG (SEQ ID NO: 266), SWSGSS (SEQ ID NO: 47), WSGS (SEQ ID NO: 267), RWSGGR (SEQ ID NO: 48), WSGG (SEQ ID NO: 268), TWNGGS (SEQ ID NO: 49), WNGG (SEQ ID NO: 269), IDWNGRGTYY (SEQ ID NO: 50), IDWNGRGTYYR (SEQ ID NO: 270), INGGGIT (SEQ ID NO: 51), IDRIATT (SEQ ID NO: 52), ITWNGGTT (SEQ ID NO: 53), ISGGGTT (SEQ ID NO: 54), ITGGGST (SEQ ID NO: 55), ISWSGGST (SEQ ID NO: 56), ISWSGSSA (SEQ ID NO: 57), IRWSGGRT (SEQ ID NO: 58), ITWNGGST (SEQ ID NO: 59), AIDWNGRGTYYRYYADSVKG (SEQ ID NO: 184), RINGGGITHYAESVKG (SEQ ID NO: 185), FIDRIATTTIATSVKG (SEQ ID NO: 186), AITWNGGTTYYADSVKG (SEQ ID NO: 187), FISGGGTTTYADSVKG (SEQ ID NO: 188), RITGGGSTHYAESVKG (SEQ ID NO: 189), AISWSGGSTTYADPVKG (SEQ ID NO: 190), AISWSGSSAGYGDSVKG (SEQ ID NO: 191), AIRWSGGRTLYADSVKG (SEQ ID NO: 192), SITWNGGSTSYADSVKG (SEQ ID NO: 193), FVAAIDWNGRGTYYRY (SEQ ID NO: 194), LVARINGGGITH (SEQ ID NO: 195), WVGFIDRIATTT (SEQ ID NO: 196), FVAAITWNGGTTY (SEQ ID NO: 197), WVAFISGGGTTT (SEQ ID NO: 198), LVARITGGGSTH (SEQ ID NO: 199), FVAAISWSGGSTT (SEQ ID NO: 200), FVAAISWSGSSAG (SEQ ID NO: 201), FVAAIRWSGGRTL (SEQ ID NO: 202), FVASITWNGGSTS (SEQ ID NO: 203), AIDWNGRGTYYRY (SEQ ID NO: 204), RINGGGITH (SEQ ID NO: 205), FIDRIATTT (SEQ ID NO: 206), AITWNGGTTY (SEQ ID NO: 207), FISGGGTTT (SEQ ID NO: 208), RITGGGSTH (SEQ ID NO: 209), AISWSGGSTT (SEQ ID NO: 210), AISWSGSSAG (SEQ ID NO: 211), AIRWSGGRTL (SEQ ID NO: 212), or SITWNGGSTS (SEQ ID NO: 213).

311. The system of any one of claims 284 to 310, wherein the single domain antibody comprises a CDR1 sequence of SYRMG (SEQ ID NO: 1), INVMG (SEQ ID NO: 2), SNAMG (SEQ ID NO: 3), SYAMG (SEQ ID NO: 4), SDAMG (SEQ ID NO: 5), INVMG (SEQ ID NO: 6), TYRMG (SEQ ID NO: 7), RYAMG (SEQ ID NO: 8), FTTYRMG (SEQ ID NO: 258), TYRMG (SEQ ID NO: 259), FNTYAMG (SEQ ID NO: 9), GLTFSSY (SEQ ID NO: 10), GSIFSIN (SEQ ID NO: 11), GTSVSSN (SEQ ID NO: 12), GRTFSSY (SEQ ID NO: 13), GSSVSSD (SEQ ID NO: 14), RSIGSIN (SEQ ID NO: 15), GRTFSTY (SEQ ID NO: 16), GFTFTRY (SEQ ID NO: 17), GRTFTTY (SEQ ID NO: 18), GRTLSFNTY (SEQ ID NO: 19), GLTFSSYR (SEQ ID NO: 20), GSIFSINV (SEQ ID NO: 21), GTSVSSNA (SEQ ID NO: 22), GRTFSSYA (SEQ ID NO: 23), GSSVSSDA (SEQ ID NO: 24), RSIGSINV (SEQ ID NO: 25), GRTFSTYR (SEQ ID NO: 26), GFTFTRYA (SEQ ID NO: 27), GRTFTTYR (SEQ ID NO: 28), GRTLSFNTYA (SEQ ID NO: 29), GLTFSSYRMG (SEQ ID NO: 154), GSIFSINVMG (SEQ ID NO: 155), GTSVSSNAMG (SEQ ID NO: 156), GRTFSSYAMG (SEQ ID NO: 157), GSSVSSDAMG (SEQ ID NO: 158), RSIGSINVMG (SEQ ID NO: 159), GRTFSTYRMG (SEQ ID NO: 160), GFTFTRYAMG (SEQ ID NO: 161), GRTFTTYRMG (SEQ ID NO: 162), GRTLSFNTYAMG (SEQ ID NO: 163), SSYRMG (SEQ ID NO: 164), SINVMG (SEQ ID NO: 165), SSNAMG (SEQ ID NO: 166), SSYAMG (SEQ ID NO: 167), SSDAMG (SEQ ID NO: 168), SINVMG (SEQ ID NO: 169), STYRMG (SEQ ID NO: 170), TRYAMG (SEQ ID NO: 171), TTYRMG (SEQ ID NO: 172), SFNTYAMG (SEQ ID NO: 173), GLTFSSYRMG (SEQ ID NO: 174), GSIFSINVMG (SEQ ID NO: 175), GTSVSSNAMG (SEQ ID NO: 176), GRTFSSYAMG (SEQ ID NO: 177), GSSVSSDAMG (SEQ ID NO: 178), RSIGSINVMG (SEQ ID NO: 179), GRTFSTYRMG (SEQ ID NO: 180), GFTFTRYAMG (SEQ ID NO: 181), GRTFTTYRMG (SEQ ID NO: 182), or GRTLSFNTYAMG (SEQ ID NO: 183).

312. The system of any one of claims 284 to 311, wherein the single domain antibody comprises a CDR1 sequence, a CDR2 sequence, and a CDR3 sequence of the single domain antibody selected from the group consisting of:
a) VHH1:
i) the CDR1 sequence of SYRMG (SEQ ID NO: 1), the CDR2 sequence of AIDWNGRGTYYRYYADSVKG (SEQ ID NO: 30), and the CDR3 sequence of GSIDLNWYGGMDY (SEQ ID NO: 60);
ii) the CDR1 sequence of GLTFSSY (SEQ ID NO: 10), the CDR2 sequence of DWNGRGTYY (SEQ ID NO: 40) or WNGRGTY (SEQ ID NO: 260), and the CDR3 sequence of GSIDLNWYGGMDY (SEQ ID NO: 71) or SIDLNWYGGMD (SEQ ID NO: 272);
iii) the CDR1 sequence of GLTFSSYR (SEQ ID NO: 20), the CDR2 sequence of IDWNGRGTYY (SEQ ID NO: 50) or IDWNGRGTYYR (SEQ ID NO: 270), and the CDR3 sequence of CAAGSIDLNWYGGMDY (SEQ ID NO: 82) or AAGSIDLNWYGGMDY (SEQ ID NO: 283);
iv) the CDR1 sequence of GLTFSSYRMG (SEQ ID NO: 154), the CDR 2 sequence of AIDWNGRGTYYRYYADSVKG (SEQ ID NO: 184), and the CDR3 sequence of GSIDLNWYGGMDY (SEQ ID NO: 214);
v) the CDR1 sequence of SSYRMG (SEQ ID NO: 164), the CDR2 sequence of FVAAIDWNGRGTYYRY (SEQ ID NO: 194), and the CDR3 sequence of AAGSIDLNWYGGMD (SEQ ID NO: 225); or
vi) the CDR1 sequence of GLTFSSYRMG (SEQ ID NO: 174), the CDR2 sequence of AIDWNGRGTYYRY (SEQ ID NO: 204), and the CDR3 sequence of GSIDLNWYGGMDY (SEQ ID NO: 236);
b) VHH2:
i) the CDR1 sequence of SYRMG (SEQ ID NO: 1), the CDR2 sequence of AIDWNGRGTYYRYYADSVKG (SEQ ID NO: 30), and the CDR3 sequence of TTVLTDPRVLNEYAT (SEQ ID NO: 61);
ii) the CDR1 sequence of GLTFSSY (SEQ ID NO: 10), the CDR2 sequence of DWNGRGTYY (SEQ ID NO: 40) or WNGRGTY (SEQ ID NO: 260), and the CDR3 sequence of TTVLTDPRVLNEYAT (SEQ ID NO: 72) or TVLTDPRVLNEYA (SEQ ID NO: 273);
iii) the CDR1 sequence of GLTFSSYR (SEQ ID NO: 20), the CDR2 sequence of IDWNGRGTYY (SEQ ID NO: 50) or IDWNGRGTYYR (SEQ ID NO: 270), and the CDR3 sequence of CAATTVLTDPRVLNEYAT (SEQ ID NO: 83) or AATTVLTDPRVLNEYAT (SEQ ID NO: 284);
iv) the CDR1 sequence of GLTFSSYRMG (SEQ ID NO: 154), the CDR2 sequence of AIDWNGRGTYYRYYADSVKG (SEQ ID NO: 184), and the CDR3 sequence of TTVLTDPRVLNEYAT (SEQ ID NO: 215);
v) the CDR1 sequence of SSYRMG (SEQ ID NO: 164), the CDR2 sequence of FVAAIDWNGRGTYYRY (SEQ ID NO: 194), and the CDR3 sequence of AATTVLTDPRVLNEYA (SEQ ID NO: 226); or
vi) the CDR1 sequence of GLTFSSYRMG (SEQ ID NO: 174), the CDR2 sequence of AIDWNGRGTYYRY (SEQ ID NO: 204), and the CDR3 sequence of TTVLTDPRVLNEYAT (SEQ ID NO: 237);
c) VHH3:
i) the CDR1 sequence of INVMG (SEQ ID NO: 2), the CDR2 sequence of RINGGGITHYAESVKG (SEQ ID NO: 31), and the CDR3 sequence of DVFGSSGYVETY (SEQ ID NO: 62);
ii) the CDR1 sequence of GSIFSIN (SEQ ID NO: 11), the CDR2 sequence of NGGGI (SEQ ID NO: 41) or GGG (SEQ ID NO: 261), and the CDR3 sequence of DVFGSSGYVETY (SEQ ID NO: 73) or VFGSSGYVET (SEQ ID NO: 274);
iii) the CDR1 sequence of GSIFSINV (SEQ ID NO: 21), the CDR2 sequence of INGGGIT (SEQ ID NO: 51), and the CDR3 sequence of KADVFGSSGYVETY (SEQ ID NO: 84);
iv) the CDR1 sequence of GSIFSINVMG (SEQ ID NO: 155), the CDR2 sequence of RINGGGITHYAESVKG (SEQ ID NO: 185), and the CDR3 sequence of DVFGSSGYVETY (SEQ ID NO: 216);
v) the CDR1 sequence of SINVMG (SEQ ID NO: 165), the CDR2 sequence of LVARINGGGITH (SEQ ID NO: 195), and the CDR3 sequence of KADVFGSSGYVET (SEQ ID NO: 227); or
vi) the CDR1 sequence of GSIFSINVMG (SEQ ID NO: 175), the CDR2 sequence of RINGGGITH (SEQ ID NO: 205), and the CDR3 sequence of DVFGSSGYVETY (SEQ ID NO: 238);
d) VHH4:
i) the CDR1 sequence of SNAMG (SEQ ID NO: 3), the CDR2 sequence of FIDRIATTTIATSVKG (SEQ ID NO: 32), and the CDR3 sequence of PLTAR (SEQ ID NO: 63);
ii) the CDR1 sequence of GTSVSSN (SEQ ID NO: 12), the CDR2 sequence of DRIAT (SEQ ID NO: 42) or RIA (SEQ ID NO: 262), and the CDR3 sequence of PLTAR (SEQ ID NO: 74) or LTA (SEQ ID NO: 275);
iii) the CDR1 sequence of GTSVSSNA (SEQ ID NO: 22), the CDR2 sequence of IDRIATT (SEQ ID NO: 52), and the CDR3 sequence of NHPLTAR (SEQ ID NO: 85);
iv) the CDR1 sequence of GTSVSSNAMG (SEQ ID NO: 156), the CDR2 sequence of FIDRIATTTIATSVKG (SEQ ID NO: 186), and the CDR3 sequence of PLTAR (SEQ ID NO: 217);
v) the CDR1 sequence of SSNAMG (SEQ ID NO: 166), the CDR2 sequence of WVGFIDRIATTT (SEQ ID NO: 196), and the CDR3 sequence of NHPLTA (SEQ ID NO: 228); or
vi) the CDR1 sequence of GTSVSSNAMG (SEQ ID NO: 176), the CDR2 sequence of FIDRIATTT (SEQ ID NO: 206), and the CDR3 sequence of PLTAR (SEQ ID NO: 239);
e) VHH5:
i) the CDR1 sequence of SYAMG (SEQ ID NO: 4), the CDR2 sequence of AITWNGGTTYYADSVKG (SEQ ID NO: 33), and the CDR3 sequence of DPFNQGY (SEQ ID NO: 64);
ii) the CDR1 sequence of GRTFSSY (SEQ ID NO: 13), the CDR2 sequence of TWNGGT (SEQ ID NO: 43) or WNGG (SEQ ID NO: 263), and the CDR3 sequence of DPFNQGY (SEQ ID NO: 75) or PFNQG (SEQ ID NO: 276);
iii) the CDR1 sequence of GRTFSSYA (SEQ ID NO: 23), the CDR2 sequence of ITWNGGTT (SEQ ID NO: 53), and the CDR3 sequence of AADPFNQGY (SEQ ID NO: 86);
iv) the CDR1 sequence of GRTFSSYAMG (SEQ ID NO: 157), the CDR2 sequence of AITWNGGTTYYADSVKG (SEQ ID NO: 187), and the CDR3 sequence of DPFNQGY (SEQ ID NO: 218);
v) the CDR1 sequence of SSYAMG (SEQ ID NO: 167), the CDR2 sequence of FVAAITWNGGTTY (SEQ ID NO: 197), and the CDR3 sequence of AADPFNQG (SEQ ID NO: 229); or
vi) the CDR1 sequence of GRTFSSYAMG (SEQ ID NO: 177), the CDR2 sequence of AITWNGGTTY (SEQ ID NO: 207), and the CDR3 sequence of DPFNQGY (SEQ ID NO: 240);
f) VHH6:
i) the CDR1 sequence of SDAMG (SEQ ID NO: 5), the CDR2 sequence of FISGGGTTTYADSVKG (SEQ ID NO: 34), and the CDR3 sequence of PLTSR (SEQ ID NO: 65);
ii) the CDR1 sequence of GSSVSSD (SEQ ID NO: 14), the CDR2 sequence of SGGGT (SEQ ID NO: 44) or GGG (SEQ ID NO: 264), and the CDR3 sequence of PLTSR (SEQ ID NO: 76) or LTS (SEQ ID NO: 277);
iii) the CDR1 sequence of GSSVSSDA (SEQ ID NO: 24), the CDR2 sequence of ISGGGTT (SEQ ID NO: 54), and the CDR3 sequence of NHPLTSR (SEQ ID NO: 87);
iv) the CDR1 sequence of GSSVSSDAMG (SEQ ID NO: 158), the CDR2 sequence of FISGGGTTTYADSVKG (SEQ ID NO: 188), and the CDR3 sequence of PLTSR (SEQ ID NO: 219);
v) the CDR1 sequence of SSDAMG (SEQ ID NO: 168), the CDR2 sequence of WVAFISGGGTTT (SEQ ID NO: 198), and the CDR3 sequence of NHPLTS (SEQ ID NO: 230); or
vi) the CDR1 sequence of GSSVSSDAMG (SEQ ID NO: 178), the CDR2 sequence of FISGGGTTT (SEQ ID NO: 208), and the CDR3 sequence of PLTSR (SEQ ID NO: 241);
g) VHH7:
i) the CDR1 sequence of INVMG (SEQ ID NO: 6), the CDR2 sequence of RITGGGSTHYAESVKG (SEQ ID NO: 35), and the CDR3 sequence of MVNPIITAWGTIGVREIPDYDY (SEQ ID NO: 66);
ii) the CDR1 sequence of RSIGSIN (SEQ ID NO: 15), the CDR2 sequence of TGGGS (SEQ ID NO: 45) or GGG (SEQ ID NO: 265), and the CDR3 sequence of MVNPIITAWGTIGVREIPDYDY (SEQ ID NO: 77) or VNPIITAWGTIGVREIPDYD (SEQ ID NO: 278);
iii) the CDR1 sequence of RSIGSINV (SEQ ID NO: 25), the CDR2 sequence of ITGGGST (SEQ ID NO: 55), and the CDR3 sequence of ASMVNPIITAWGTIGVREIPDYDY (SEQ ID NO: 88);
iv) the CDR1 sequence of RSIGSINVMG (SEQ ID NO: 159), the CDR2 sequence of RITGGGSTHYAESVKG (SEQ ID NO: 189), and the CDR3 sequence of MVNPIITAWGTIGVREIPDYDY (SEQ ID NO: 220);
v) the CDR1 sequence of SINVMG (SEQ ID NO: 169), the CDR2 sequence of LVARITGGGSTH (SEQ ID NO: 199), and the CDR3 sequence of ASMVNPIITAWGTIGVREIPDYD (SEQ ID NO: 231); or
vi) the CDR1 sequence of RSIGSINVMG (SEQ ID NO: 179), the CDR2 sequence of RITGGGSTH (SEQ ID NO: 209), and the CDR3 sequence of MVNPIITAWGTIGVREIPDYDY (SEQ ID NO: 242);
h) VHH9:
i) the CDR1 sequence of TYRMG (SEQ ID NO: 7), the CDR2 sequence of AISWSGGSTTYADPVKG (SEQ ID NO: 36), and the CDR3 sequence of DQRGY (SEQ ID NO: 67) or QRGY (SEQ ID NO: 271);
ii) the CDR1 sequence of GRTFSTY (SEQ ID NO: 16), the CDR2 sequence of SWSGGS (SEQ ID NO: 46) or WSGG (SEQ ID NO: 266), and the CDR3 sequence of DQRGY (SEQ ID NO: 78) or RG (SEQ ID NO: 279);
iii) the CDR1 sequence of GRTFSTYR (SEQ ID NO: 26), the CDR2 sequence of ISWSGGST (SEQ ID NO: 56), and the CDR3 sequence of NDQRGY (SEQ ID NO: 89);
iv) the CDR1 sequence of GRTFSTYRMG (SEQ ID NO: 160), the CDR2 sequence of AISWSGGSTTYADPVKG (SEQ ID NO: 190), and the CDR3 sequence of QRGY (SEQ ID NO: 221);
v) the CDR1 sequence of STYRMG (SEQ ID NO: 170), the CDR2 sequence of FVAAISWSGGSTT (SEQ ID NO: 200), and the CDR3 sequence of NDQRG (SEQ ID NO: 232); or
vi) the CDR1 sequence of GRTFSTYRMG (SEQ ID NO: 180), the CDR2 sequence of AISWSGGSTT (SEQ ID NO: 210), and the CDR3 sequence of QRGY (SEQ ID NO: 243);
i) VHH10:
i) the CDR1 sequence of RYAMG (SEQ ID NO: 8), the CDR2 sequence of AISWSGSSAGYGDSVKG (SEQ ID NO: 37), and the CDR3 sequence of DPFNQGY (SEQ ID NO: 68);
ii) the CDR1 sequence of GFTFTRY (SEQ ID NO: 17), the CDR2 sequence of SWSGSS (SEQ ID NO: 47) or WSGS (SEQ ID NO: 267), and the CDR3 sequence of DPFNQGY (SEQ ID NO: 79) or PFNQG (SEQ ID NO: 280);
iii) the CDR1 sequence of GFTFTRYA (SEQ ID NO: 27), the CDR2 sequence of ISWSGSSA (SEQ ID NO: 57), and the CDR3 sequence of AADPFNQGY (SEQ ID NO: 90);
iv) the CDR1 sequence of GFTFTRYAMG (SEQ ID NO: 161), the CDR2 sequence of AISWSGSSAGYGDSVKG (SEQ ID NO: 191), and the CDR3 sequence of DPFNQGY (SEQ ID NO: 222);
v) the CDR1 sequence of TRYAMG (SEQ ID NO: 171), the CDR2 sequence of FVAAISWSGSSAG (SEQ ID NO: 201), and the CDR3 sequence of AADPFNQG (SEQ ID NO: 233); or
vi) the CDR1 sequence of GFTFTRYAMG (SEQ ID NO: 181), the CDR2 sequence of AISWSGSSAG (SEQ ID NO: 211), and the CDR3 sequence of DPFNQGY (SEQ ID NO: 244);
j) VHH11:
i) the CDR1 sequence of FTTYRMG (SEQ ID NO: 258) or TYRMG (SEQ ID NO: 259), the CDR2 sequence of AIRWSGGRTLYADSVKG (SEQ ID NO: 38), and the CDR3 sequence of DLAEYSGTYSSPADSPAGYDY (SEQ ID NO: 69);
ii) the CDR1 sequence of GRTFTTY (SEQ ID NO: 18), the CDR2 sequence of RWSGGR (SEQ ID NO: 48) or WSGG (SEQ ID NO: 268), and the CDR3 sequence of DLAEYSGTYSSPADSPAGYDY (SEQ ID NO: 80) or LAEYSGTYSSPADSPAGYD (SEQ ID NO: 281);
iii) the CDR1 sequence of GRTFTTYR (SEQ ID NO: 28), the CDR2 sequence of IRWSGGRT (SEQ ID NO: 58), and the CDR3 sequence of AADLAEYSGTYSSPADSPAGYDY (SEQ ID NO: 91);
iv) the CDR1 sequence of GRTFTTYRMG (SEQ ID NO: 162), the CDR2 sequence of AIRWSGGRTLYADSVKG (SEQ ID NO: 192), and the CDR3 sequence of DLAEYSGTYSSPADSPAGYDY (SEQ ID NO: 223);
v) the CDR1 sequence of TTYRMG (SEQ ID NO: 172), the CDR2 sequence of FVAAIRWSGGRTL (SEQ ID NO: 202), and the CDR3 sequence of AADLAEYSGTYSSPADSPAGYD (SEQ ID NO: 234); or
vi) the CDR1 sequence of GRTFTTYRMG (SEQ ID NO: 182), the CDR2 sequence of AIRWSGGRTL (SEQ ID NO: 212), and the CDR3 sequence of DLAEYSGTYSSPADSPAGYDY (SEQ ID NO: 245); and
k) VHH12:
i) the CDR1 sequence of FNTYAMG (SEQ ID NO: 9), the CDR2 sequence of SITWNGGSTSYADSVKG (SEQ ID NO: 39), and the CDR3 sequence of ARYYVSGTYFPANY (SEQ ID NO: 70);
ii) the CDR1 sequence of GRTLSFNTY (SEQ ID NO: 19), the CDR2 sequence of TWNGGS (SEQ ID NO: 49) or WNGG (SEQ ID NO: 269), and the CDR3 sequence of ARYYVSGTYFPANY (SEQ ID NO: 81) or RYYVSGTYFPAN (SEQ ID NO: 282);
iii) the CDR1 sequence of GRTLSFNTYA (SEQ ID NO: 29), the CDR2 sequence of ITWNGGST (SEQ ID NO: 59), and the CDR3 sequence of AAARYYVSGTYFPANY (SEQ ID NO: 92);
iv) the CDR1 sequence of GRTLSFNTYAMG (SEQ ID NO: 163), the CDR2 sequence of SITWNGGSTSYADSVKG (SEQ ID NO: 193), and the CDR3 sequence of ARYYVSGTYFPANY (SEQ ID NO: 224);
v) the CDR1 sequence of SFNTYAMG (SEQ ID NO: 173), the CDR2 sequence of FVASITWNGGSTS (SEQ ID NO: 203), and the CDR3 sequence of AAARYYVSGTYFPAN (SEQ ID NO: 235); or
vi) the CDR1 sequence of GRTLSFNTYAMG (SEQ ID NO: 183), the CDR2 sequence of SITWNGGSTS (SEQ ID NO: 213), and the CDR3 sequence of ARYYVSGTYFPANY (SEQ ID NO: 246).

313. The system of any one of claims 284 to 312, wherein the single domain antibody comprises a framework derived from the framework of any of the single domain antibodies comprising the sequences of
(SEQ ID NO: 93)
QVQLVESGGGLVQAGGSLKLACAAPGLTFSSYRMGWFRQAPGQEREFVA
AIDWNGRGTYYRYYADSVKGRSTISRDNAKNTMYLQMNSLKPEDTAVYY
CAAGSIDLNWYGGMDYWGQGTQVTVSS,
(SEQ ID NO: 94)
EVQVVESGGGLVQAGGSLKLACAAPGLTFSSYRMGWFRQAPGQEREFVA
AIDWNGRGTYYRYYADSVKGRSTISRDNAKNTVYLQMNSLKPEDTAVYY
CAATTVLTDPRVLNEYATWGQGTQVTVSS,
(SEQ ID NO: 95)
QLQLVESGGGLVQPGGSLRLSCAASGSIFSINVMGWYRQAPGKQRELVA
RINGGGITHYAESVKGRFTISRDNAKNTVYLQMNSLKPEDTAAYYCKAD
VFGSSGYVETYWGQGTQVTVSS,
(SEQ ID NO: 96)
EVQVVESGGGLVQAGGSLRLSCAVSGTSVSSNAMGWYRQAPGKQREWVG
FIDRIATTTIATSVKGRFAITRDNAKNTVYLQMSGLKPEDTAVYYCNHP
LTARWGQGTQVTVSS,
(SEQ ID NO: 97)
QVQLVESGGGLVQAGGSLRLSCAASGRTFSSYAMGWFRQAPGKEREFVA
AITWNGGTTYYADSVKGRFTISRDNAKNTVYLQMNSLKPEDTAVYYCAA
DPFNQGYWGQGTQVTVSS,
(SEQ ID NO: 98)
EVQLVESGGGLVQAGGSLRLSCAVSGSSVSSDAMGWYRQAPGNQRAWVA
FISGGGTTTYADSVKGRFTISRDNTKNTVYLHMNSLKPEDTAVYYCNHP
LTSRWGQGTQVTVSS,
(SEQ ID NO: 99)
EVQVVESGGGLVQAGGSLRLACVASRSIGSINVMGWYRQAPGKQRDLVA
RITGGGSTHYAESVKGRFTISRDNAKNTVYLQMNSLEPEDTAVYYCASM
VNPIITAWGTIGVREIPDYDYWGQGTQVTVSS,
(SEQ ID NO: 100)
QVQLVESGGGLVQAGGSLRLSCAVSGRTFSTYRMGWFRQAPGKERSFVA
AISWSGGSTTYADPVKGRFTISRDNAKNTVYLRMNSLKPEDTAVYYCND
QRGYWGQGTLVTVSS,
(SEQ ID NO: 101)
EVQVVESGGGLVQAGGSLRLSCAASGFTFTRYAMGWFRQAPGKERSFVA
AISWSGSSAGYGDSVKGRFTISRDNAKNTLYLQMNSLKPEDTAVYYCAA
DPFNQGYWGQGTQVTVSS,
(SEQ ID NO: 102)
EVQVVESGGGLVQAGGSLRLSCAASGRTFTTYRMGWFRQAPGKEREFVA
AIRWSGGRTLYADSVKGRFTISRDNAKNTAYLQMNNLRPEDTAVYYCAA
DLAEYSGTYSSPADSPAGYDYWGQGTQVTVSS,
or
(SEQ ID NO: 103)
QVQLVETGGGLVQAGDSLRLSCAASGRTLSFNTYAMGWFRQAPGKEREF
VASITWNGGSTSYADSVKGRFTITRDNAKNTATLRMNSLQPDDTAVYYC
AAARYYVSGTYFPANYWGQGTQVTVSS.

314. The system of any one of claims 284 to 312, wherein the single domain antibody comprises a framework comprising sequence having at least 75%, 80%, 85%, 90%, 95%, 96%, 97%, 98%, 99%, or 100% sequence identity with the sequence of
(SEQ ID NO: 93)
QVQLVESGGGLVQAGGSLKLACAAPGLTFSSYRMGWFRQAPGQEREFVA
AIDWNGRGTYYRYYADSVKGRSTISRDNAKNTMYLQMNSLKPEDTAVYY
CAAGSIDLNWYGGMDYWGQGTQVTVSS,
(SEQ ID NO: 94)
EVQVVESGGGLVQAGGSLKLACAAPGLTFSSYRMGWFRQAPGQEREFVA
AIDWNGRGTYYRYYADSVKGRSTISRDNAKNTVYLQMNSLKPEDTAVYY
CAATTVLTDPRVLNEYATWGQGTQVTVSS,
(SEQ ID NO: 95)
QLQLVESGGGLVQPGGSLRLSCAASGSIFSINVMGWYRQAPGKQRELVA
RINGGGITHYAESVKGRFTISRDNAKNTVYLQMNSLKPEDTAAYYCKAD
VFGSSGYVETYWGQGTQVTVSS,
(SEQ ID NO: 96)
EVQVVESGGGLVQAGGSLRLSCAVSGTSVSSNAMGWYRQAPGKQREWVG
FIDRIATTTIATSVKGRFAITRDNAKNTVYLQMSGLKPEDTAVYYCNHP
LTARWGQGTQVTVSS,
(SEQ ID NO: 97)
QVQLVESGGGLVQAGGSLRLSCAASGRTFSSYAMGWFRQAPGKEREFVA
AITWNGGTTYYADSVKGRFTISRDNAKNTVYLQMNSLKPEDTAVYYCAA
DPFNQGYWGQGTQVTVSS,
(SEQ ID NO: 98)
EVQLVESGGGLVQAGGSLRLSCAVSGSSVSSDAMGWYRQAPGNQRAWVA
FISGGGTTTYADSVKGRFTISRDNTKNTVYLHMNSLKPEDTAVYYCNHP
LTSRWGQGTQVTVSS,
(SEQ ID NO: 99)
EVQVVESGGGLVQAGGSLRLACVASRSIGSINVMGWYRQAPGKQRDLVA
RITGGGSTHYAESVKGRFTISRDNAKNTVYLQMNSLEPEDTAVYYCASM
VNPIITAWGTIGVREIPDYDYWGQGTQVTVSS,
(SEQ ID NO: 100)
QVQLVESGGGLVQAGGSLRLSCAVSGRTFSTYRMGWFRQAPGKERSFVA
AISWSGGSTTYADPVKGRFTISRDNAKNTVYLRMNSLKPEDTAVYYCND
QRGYWGQGTLVTVSS,
(SEQ ID NO: 101)
EVQVVESGGGLVQAGGSLRLSCAASGFTFTRYAMGWFRQAPGKERSFVA
AISWSGSSAGYGDSVKGRFTISRDNAKNTLYLQMNSLKPEDTAVYYCAA
DPFNQGYWGQGTQVTVSS,
(SEQ ID NO: 102)
EVQVVESGGGLVQAGGSLRLSCAASGRTFTTYRMGWFRQAPGKEREFVA
AIRWSGGRTLYADSVKGRFTISRDNAKNTAYLQMNNLRPEDTAVYYCAA
DLAEYSGTYSSPADSPAGYDYWGQGTQVTVSS,
or
(SEQ ID NO: 103)
QVQLVETGGGLVQAGDSLRLSCAASGRTLSFNTYAMGWFRQAPGKEREF
VASITWNGGSTSYADSVKGRFTITRDNAKNTATLRMNSLQPDDTAVYYC
AAARYYVSGTYFPANYWGQGTQVTVSS.

315. The system of any one of claims 284 to 314, wherein the single domain antibody is comprised of a sequence having at least 75%, 80%, 85%, 90%, 95%, 96%, 97%, 98%, 99%, or 100% sequence identity with the sequence of
(SEQ ID NO: 93)
QVQLVESGGGLVQAGGSLKLACAAPGLTFSSYRMGWFRQAPGQEREFVA
AIDWNGRGTYYRYYADSVKGRSTISRDNAKNTMYLQMNSLKPEDTAVYY
CAAGSIDLNWYGGMDYWGQGTQVTVSS,
(SEQ ID NO: 94)
EVQVVESGGGLVQAGGSLKLACAAPGLTFSSYRMGWFRQAPGQEREFVA
AIDWNGRGTYYRYYADSVKGRSTISRDNAKNTVYLQMNSLKPEDTAVYY
CAATTVLTDPRVLNEYATWGQGTQVTVSS,
(SEQ ID NO: 95)
QLQLVESGGGLVQPGGSLRLSCAASGSIFSINVMGWYRQAPGKQRELVA
RINGGGITHYAESVKGRFTISRDNAKNTVYLQMNSLKPEDTAAYYCKAD
VFGSSGYVETYWGQGTQVTVSS,
(SEQ ID NO: 96)
EVQVVESGGGLVQAGGSLRLSCAVSGTSVSSNAMGWYRQAPGKQREWVG
FIDRIATTTIATSVKGRFAITRDNAKNTVYLQMSGLKPEDTAVYYCNHP
LTARWGQGTQVTVSS,
(SEQ ID NO: 97)
QVQLVESGGGLVQAGGSLRLSCAASGRTFSSYAMGWFRQAPGKEREFVA
AITWNGGTTYYADSVKGRFTISRDNAKNTVYLQMNSLKPEDTAVYYCAA
DPFNQGYWGQGTQVTVSS,
(SEQ ID NO: 98)
EVQLVESGGGLVQAGGSLRLSCAVSGSSVSSDAMGWYRQAPGNQRAWVA
FISGGGTTTYADSVKGRFTISRDNTKNTVYLHMNSLKPEDTAVYYCNHP
LTSRWGQGTQVTVSS,
(SEQ ID NO: 99)
EVQVVESGGGLVQAGGSLRLACVASRSIGSINVMGWYRQAPGKQRDLVA
RITGGGSTHYAESVKGRFTISRDNAKNTVYLQMNSLEPEDTAVYYCASM
VNPIITAWGTIGVREIPDYDYWGQGTQVTVSS,
(SEQ ID NO: 100)
QVQLVESGGGLVQAGGSLRLSCAVSGRTFSTYRMGWFRQAPGKERSFVA
AISWSGGSTTYADPVKGRFTISRDNAKNTVYLRMNSLKPEDTAVYYCND
QRGYWGQGTLVTVSS,
(SEQ ID NO: 101)
EVQVVESGGGLVQAGGSLRLSCAASGFTFTRYAMGWFRQAPGKERSFVA
AISWSGSSAGYGDSVKGRFTISRDNAKNTLYLQMNSLKPEDTAVYYCAA
DPFNQGYWGQGTQVTVSS,
(SEQ ID NO: 102)
EVQVVESGGGLVQAGGSLRLSCAASGRTFTTYRMGWFRQAPGKEREFVA
AIRWSGGRTLYADSVKGRFTISRDNAKNTAYLQMNNLRPEDTAVYYCAA
DLAEYSGTYSSPADSPAGYDYWGQGTQVTVSS,
or
(SEQ ID NO: 103)
QVQLVETGGGLVQAGDSLRLSCAASGRTLSFNTYAMGWFRQAPGKEREF
VASITWNGGSTSYADSVKGRFTITRDNAKNTATLRMNSLQPDDTAVYYC
AAARYYVSGTYFPANYWGQGTQVTVSS.

316. The system of any one of claims 284 to 315, wherein the single domain antibody is genetically fused or chemically conjugated to the agent.

317. The system of claim 316, further comprising a linker between the single domain antibody and the agent.

318. The system of claim 317, wherein the linker is a polypeptide.

319. The system of claim 318, wherein the linker is a flexible linker comprising a sequence selected from the group consisting of EPKTPKPQPQPQLQPQPNPTTESKSPK (SEQ ID NO: 130), (EAAAK)n (SEQ ID NO: 147), (GGGGS)n (SEQ ID NO: 148) and (GGGS)n (SEQ ID NO: 149), wherein n is an integer from 1 to 20.

320. The system of any one of claims 316 to 319, wherein the single domain antibody is chemically-conjugated to the agent.

321. The system of any one of claims 316 to 319, wherein the single domain antibody is non-covalently bound to the agent.

322. The system of any one of claims 284 to 321, wherein the system does not inhibit pIgR-mediated transcytosis of IgA.

323. The system of claim 322, wherein the single domain antibody comprises a CDR1 sequence of SNAMG (SEQ ID NO: 3), INVMG (SEQ ID NO: 6), TYRMG (SEQ ID NO: 7), RYAMG (SEQ ID NO: 8), FTTYRMG (SEQ ID NO: 258), TYRMG (SEQ ID NO: 259), FNTYAMG (SEQ ID NO: 9), GTSVSSN (SEQ ID NO: 12), GRTFSSY (SEQ ID NO: 13), RSIGSIN (SEQ ID NO: 15), GRTFSTY (SEQ ID NO: 16), GFTFTRY (SEQ ID NO: 17), GRTFTTY (SEQ ID NO: 18), GRTLSFNTY (SEQ ID NO: 19), GTSVSSNA (SEQ ID NO: 22), RSIGSINV (SEQ ID NO: 25), GRTFSTYR (SEQ ID NO: 26), GFTFTRYA (SEQ ID NO: 27), GRTFTTYR (SEQ ID NO: 28), GRTLSFNTYA (SEQ ID NO: 29), GTSVSSNAMG (SEQ ID NO: 156), RSIGSINVMG (SEQ ID NO: 159), GRTFSTYRMG (SEQ ID NO: 160), GFTFTRYAMG (SEQ ID NO: 161), GRTFTTYRMG (SEQ ID NO: 162), GRTLSFNTYAMG (SEQ ID NO: 163), SSNAMG (SEQ ID NO: 166), SINVMG (SEQ ID NO: 169), STYRMG (SEQ ID NO: 170), TRYAMG (SEQ ID NO: 171), TTYRMG (SEQ ID NO: 172), SFNTYAMG (SEQ ID NO: 173), GTSVSSNAMG (SEQ ID NO: 176), RSIGSINVMG (SEQ ID NO: 179), GRTFSTYRMG (SEQ ID NO: 180), GFTFTRYAMG (SEQ ID NO: 181), GRTFTTYRMG (SEQ ID NO: 182), or GRTLSFNTYAMG (SEQ ID NO: 183).

324. The system of claim 322 or claim 323, wherein the single domain antibody comprises a CDR2 sequence of FIDRIATTTIATSVKG (SEQ ID NO: 32), RITGGGSTHYAESVKG (SEQ ID NO: 35), AISWSGGSTTYADPVKG (SEQ ID NO: 36), AISWSGSSAGYGDSVKG (SEQ ID NO: 37), AIRWSGGRTLYADSVKG (SEQ ID NO: 38), SITWNGGSTSYADSVKG (SEQ ID NO: 39), DRIAT (SEQ ID NO: 42), RIA (SEQ ID NO: 262), TGGGS (SEQ ID NO: 45), GGG (SEQ ID NO: 265), SWSGGS (SEQ ID NO: 46), WSGG (SEQ ID NO: 266), SWSGSS (SEQ ID NO: 47), WSGS (SEQ ID NO: 267), RWSGGR (SEQ ID NO: 48), WSGG (SEQ ID NO: 268), TWNGGS (SEQ ID NO: 49), WNGG (SEQ ID NO: 269), IDRIATT (SEQ ID NO: 52), ITGGGST (SEQ ID NO: 55), ISWSGGST (SEQ ID NO: 56), ISWSGSSA (SEQ ID NO: 57), IRWSGGRT (SEQ ID NO: 58), ITWNGGST (SEQ ID NO: 59), FIDRIATTTIATSVKG (SEQ ID NO: 186), RITGGGSTHYAESVKG (SEQ ID NO: 189), AISWSGGSTTYADPVKG (SEQ ID NO: 190), AISWSGSSAGYGDSVKG (SEQ ID NO: 191), AIRWSGGRTLYADSVKG (SEQ ID NO: 192), SITWNGGSTSYADSVKG (SEQ ID NO: 193), WVGFIDRIATTT (SEQ ID NO: 196), LVARITGGGSTH (SEQ ID NO: 199), FVAAISWSGGSTT (SEQ ID NO: 200), FVAAISWSGSSAG (SEQ ID NO: 201), FVAAIRWSGGRTL (SEQ ID NO: 202), FVASITWNGGSTS (SEQ ID NO: 203), FIDRIATTT (SEQ ID NO: 206), RITGGGSTH (SEQ ID NO: 209), AISWSGGSTT (SEQ ID NO: 210), AISWSGSSAG (SEQ ID NO: 211), AIRWSGGRTL (SEQ ID NO: 212), or SITWNGGSTS (SEQ ID NO: 213).

325. The system of any one of claims 322 to 324, wherein the single domain antibody comprises a CDR3 sequence of PLTAR (SEQ ID NO: 63), MVNPIITAWGTIGVREIPDYDY (SEQ ID NO: 66), DQRGY (SEQ ID NO: 67), QRGY (SEQ ID NO: 271), DPFNQGY (SEQ ID NO: 68), DLAEYSGTYSSPADSPAGYDY (SEQ ID NO: 69), ARYYVSGTYFPANY (SEQ ID NO: 70), PLTAR (SEQ ID NO: 74), LTA (SEQ ID NO: 275), MVNPIITAWGTIGVREIPDYDY (SEQ ID NO: 77), VNPIITAWGTIGVREIPDYD (SEQ ID NO: 278), DQRGY (SEQ ID NO: 78), RG (SEQ ID NO: 279), DPFNQGY (SEQ ID NO: 79), PFNQG (SEQ ID NO: 280), DLAEYSGTYSSPADSPAGYDY (SEQ ID NO: 80), LAEYSGTYSSPADSPAGYD (SEQ ID NO: 281), ARYYVSGTYFPANY (SEQ ID NO: 81), RYYVSGTYFPAN (SEQ ID NO: 282), NHPLTAR (SEQ ID NO: 85), ASMVNPIITAWGTIGVREIPDYDY (SEQ ID NO: 88), NDQRGY (SEQ ID NO: 89), AADPFNQGY (SEQ ID NO: 90), AADLAEYSGTYSSPADSPAGYDY (SEQ ID NO: 91), AAARYYVSGTYFPANY (SEQ ID NO: 92), PLTAR (SEQ ID NO: 217), MVNPIITAWGTIGVREIPDYDY (SEQ ID NO: 220), QRGY (SEQ ID NO: 221), DPFNQGY (SEQ ID NO: 222), DLAEYSGTYSSPADSPAGYDY (SEQ ID NO: 223), ARYYVSGTYFPANY (SEQ ID NO: 224), NHPLTA (SEQ ID NO: 228), ASMVNPIITAWGTIGVREIPDYD (SEQ ID NO: 231), NDQRG (SEQ ID NO: 232), AADPFNQG (SEQ ID NO: 233), AADLAEYSGTYSSPADSPAGYD (SEQ ID NO: 234), AAARYYVSGTYFPAN (SEQ ID NO: 235), PLTAR (SEQ ID NO: 239), MVNPIITAWGTIGVREIPDYDY (SEQ ID NO: 242), QRGY (SEQ ID NO: 243), DPFNQGY (SEQ ID NO: 244), DLAEYSGTYSSPADSPAGYDY (SEQ ID NO: 245), or ARYYVSGTYFPANY (SEQ ID NO: 246).

326. A system comprising a means for providing a molecule to Lamina propria of a subject.

327. The system of claim 326, wherein the molecule comprises an agent and a single domain antibody that binds to pIgR.

328. The system of claim 327, wherein the agent is an antibody or fragment thereof, a peptide, a vaccine, a small molecule, a polynucleotide, a radioisotope, a toxin, an enzyme, an anticoagulant, a hormone, a cytokine, an anti-inflammatory molecule, an RNAi, an antibiotic, or an antibody-antibiotic conjugate.

329. The system of any one of claims 326 to 328, wherein the agent is an antibody or fragment thereof, a peptide, or a vaccine.

330. The system of any one of claims 327 to 329, wherein the single domain antibody is genetically fused or chemically conjugated to the agent.
